(19) United States
(12) Patent Application Publication
(10) Pub. No.: US 2007/0203083 A1
(43) Pub. Date: Aug. 30, 2007

(54) Methods Of Regulating Metabolism And Mitochondrial Function

(76) Inventors: Vamsi Krishna Mootha, Brookline, MA (US); David Altshuler, Brookline, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/560,501

(22) PCT Filed: Jun. 14, 2004
(86) PCT No.: PCT/US04/19017

§ 371 Date: Jun. 15, 2006

Related U.S. Application Data
(60) Provisional application No. 60/478,238, filed on Jun. 13, 2003.
(60) Provisional application No. 60/525,548, filed on Nov. 26, 2003.
(60) Provisional application No. 60/559,141, filed on Apr. 2, 2004.

(51) Int. Cl. A61K 48/00; C40B 30/06; C40B 40/08

(52) U.S. Cl. 514/44; 435/6

(57) ABSTRACT

The invention relates to novel methods of regulating metabolism and mitochondrial biogenesis. Some aspects of the invention relate to methods of treating or preventing diseases in a patient associated with reduced mitochondrial function, to methods of identifying agents to treat such diseases, and to methods of diagnosing such diseases. Other aspects of the invention relate to a set of coordinately-regulated genes which regulate oxidative phosphorylation.

### BACKGROUND OF THE INVENTION

Type 2 diabetes (DM2) affects an estimated 110 million people worldwide and is a major contributor to atherosclerotic vascular disease, blindness, amputation, and kidney failure. Defects in insulin secretion are observed early in patients with MODY, a monogenic form of type 2 diabetes; insulin resistance at tissues such as skeletal muscle is a cardinal feature of patients with fully developed DM2. Many molecular pathways have been implicated in the disease process: beta-cell development, insulin receptor signaling, carbohydrate production and utilization, mitochondrial metabolism, fatty acid oxidation, cytokine signaling, adipogenesis, adrenergic signaling, and others. It remains unclear, however, which of these or other pathways are disturbed in, and might be responsible for, DM2 in its common form.

Therefore, a need remains to identify the molecular pathways implicated in the disease process and to develop new tools and assays to identify therapeutics for the treatment of diabetes.

### SUMMARY OF THE INVENTION

One aspect of the invention provides a method of modulating a biological response in a cell, the method comprising contacting the cell with at least one agent that modulates the expression or activity of Errα or Gabp, wherein the biological response is (a) expression of at least one OXPHOS gene; (b) mitochondrial biogenesis; (c) expression of Nuclear Respiratory Factor 1 (NRF-1); (d) β-oxidation of fatty acids; (e) total mitochondrial respiration; (f) uncoupled respiration; (g) mitochondrial DNA replication; (h) expression of mitochondrial enzymes; or (i) skeletal muscle fiber-type switching.

Another aspect of the invention provides a method of determining if an agent is a potential agent for the treatment of a disorder that is characterized by glucose intolerance, insulin resistance or reduced mitochondrial function, the method comprising determining if the agent increases: (i) the expression or activity of Errα or Gabp in a cell; or (ii) the formation of a complex between a PGC-1 polypeptide and (1) an Errα polypeptide; or (2) a Gabp polypeptide; wherein an agent that increases (i) or (ii) is a potential target for the treatment of the disorder.

The invention also provides a method of identifying an agent that modulates a biological response, the method comprising (a) contacting, in the presence of the agent, a PGC-1 polypeptide and an (i) Errα polypeptide, or (ii) a Gabp polypeptide, under conditions which allow the formation of a complex between the PGC-1 polypeptide and (i) the Errα polypeptide, or (ii) the Gabp polypeptide; and (b) detecting the presence of the complex; wherein an agent that modulates the biological response is identified if the agent increases or decreases the formation of the complex, and wherein the biological response is (a) expression of at least one OXPHOS gene; (b) mitochondrial biogenesis; (c) expression of Nuclear Respiratory Factor 1 (NRF-1); (d) β-oxidation of fatty acids; (e) total mitochondrial respiration; (f) uncoupled respiration; (g) mitochondrial DNA replication; (h) expression of mitochondrial enzymes; or (i) skeletal muscle fiber-type switching.

Additionally, the invention provides a method of treating or preventing a disorder characterized by reduced mitochondrial function, glucose intolerance, or insulin intolerance in a subject, the method comprising administering to the subject a therapeutically effective amount of an agent which (i) increases the expression or activity of Errα or Gabp or both; or (ii) increases the formation of a complex between a PGC-1 polypeptide and (a) an Errα polypeptide; (b) a Gabp polypeptide; or both; or (iii) binds to an (a) Errα binding site, or to a (b) Gabpa binding site, and which increases transcription of at least one gene in the subject, said gene having an Errα binding site, a Gabpa binding site, or both.

Yet another aspect of the invention provides a method of treating or preventing a disorder characterized by reduced mitochondrial function, glucose intolerance, or insulin intolerance in a subject, the method comprising administering to the subject a therapeutically effective amount of an agent which increases the expression or activity of a gene, wherein the gene has an Errα binding site or a Gapba binding site.

The invention also provides a method of reducing the metabolic rate of a subject in need thereof, the method comprising administering to the subject a therapeutically effective amount of an agent which decreases the expression or activity of at least one of the following: (i) Errα; (ii) Gabpa; (iii) a gene having an Errα binding site, a Gabpa binding site, or both; or (iv) a transcriptional activator which binds to an Errα binding site or to a Gabpa binding site; thereby reducing the metabolic rate of the patient.

The invention further provides a method of identifying a susceptibility locus for a disorder that is characterized by reduced mitochondrial function, glucose intolerance, or insulin intolerance in a subject, the method comprising (i) identifying at least one polymorphisms in a gene, or linked to a gene, wherein the gene (a) has an Errα binding site, a Gabpa binding site, or both; or (b) is Errα, Gabpa, or Gabpb; (ii) determining if at least one polymorphism is associated with the incidence of the disorder, wherein if a polymorphism is associated with the incidence of the disorder then the gene having the polymorphism, or the gene to which the polymorphism is linked, is a susceptibility locus.

A related aspect of the invention provides a method of determining if a subject is at risk of developing a disorder which is characterized by reduced mitochondrial function, the method comprising determining if a gene from the subject contains a mutation which reduces the function of the gene, wherein the gene has an Errα binding site, a Gapba binding site, or both, wherein if a gene from the subject contains a mutation then the subject is at risk of developing the disorder.

Yet another aspect of the invention provides a method of identifying a transcriptional regulator having differential activity between an experimental cell and a control cell, the method comprising (i) determining the level of gene expression of at least two genes in the experimental cell and in the control cell; (ii) ranking genes according to a difference metric of their expression level in the experimental cell compared to the control cell; (iii) identifying a subset of genes, wherein each gene in the subset contains the same DNA sequence motif; (iv) testing using a nonparametric statistic if the subset of genes are enriched at either the top or the bottom of the ranking; (v) optionally reiterating steps (ii)-(iii) for additional motifs; (vi) for a subset of genes that is enriched, identifying a transcriptional regulator which binds to a DNA sequence motif that is contained in the subset of genes; thereby identifying a transcriptional regulator having differential activity between two cells.

An additional aspect of the invention provides a method of treating impaired glucose tolerance in an individual in need thereof, the method comprising administering to the individual a therapeutically effective amount of an agent which increases the expression level of at least two OXPHOS-CR genes, thereby treating impaired glucose tolerance in the individual. A related aspect provides a method of treating obesity in an individual, comprising administering to the individual a therapeutically effective amount of an agent which increases the expression level of at least two OSPHOS-CR genes, thereby treating obesity in the individual.

One aspect of the invention provides a method of detecting statistically-significant differences in the expression level of at least one biomarker belonging to a biomarker set, between the members of a first and of a second experimental group, comprising: (a) obtaining a biomarker sample from members of the first and the second experimental groups; (b) determining, for each biomarker sample, the expression levels of at least one biomarker belonging to the biomarker set and of at least one biomarker not belonging to the set; (c) generating a ranks order of each biomarker according to a difference metric of its expression level in the first experimental group compared to the second experimental group; (d) calculating an experimental enrichment score for the biomarker set by applying a non parametric statistic; and (e) comparing the experimental enrichment score with a distribution of randomized enrichment scores to calculate the fraction of randomized enrichment scores greater than the experimental enrichment score, wherein a low fraction indicates a statistically-significant difference in the expression level of the biomarker set, between the members of a first and of a second experimental group. In one embodiment, the distribution of randomized enrichment scores is generated by (i) randomly permutating the assignment of each biomarker sample to the first or to the second experimental group; (ii) generating a rank order of each biomarker according to the absolute value of a difference metric of its expression level in the first experimental group compared to the second experimental group; (iii) calculating an experimental enrichment score for the biomarker set by applying a non parametric statistic to the rank order; and (iv) repeating steps (i), (ii) and (iii) a number of times sufficient to generate the distribution of randomized enrichment scores.

In addition, the invention provides a method of identifying an agent that regulates expression of OXPHOS-CR genes, the method comprising (a) contacting (i) an agent to be assessed for its ability to regulate expression of OXPHOS-CR genes with (ii) a test cell; and (b) determining whether the expression of at least two OXPHOS-CR gene products show a coordinate change in the test cell compared to an appropriate control, wherein a coordinate change in the expression of the OXPHOS-CR gene products indicates that the agent regulates the expression levels of OXPHOS-CR genes. In one embodiment, the OXPHOS-CR genes are selected from the group consisting of NDUFB3, SDHA, NDUFA8, COX7A1, UQCRC1, NDUFC1, NDUFS2, ATP5O, NDUFS3, SDHB, NDUFS5, NDUFB6, COX5B, CYC1, NDUFA7, UQCRB, COX7B, ATP5L, COX7C, NDUFA5, GRIM19, ATP5J, COX6A2 NDUFB5, CYCS, NDUFA2 and HSPC051.

### BRIEF DESCRIPTION OF THE DRAWINGS

FIG. 1 shows a schematic overview of an embodiment of gene set enrichment analysis (GSEA). The goal of GSEA is to determine whether any a priori defined gene sets (step 1) are enriched at the top of list of genes ordered on the basis of expression difference between two classes (e.g., high in NGT vs. DM2). Genes, R₁, . . . R_{N}, are rank ordered on the basis of expression difference (step 2) using an appropriate difference measure (e.g. signal to noise ratio (SNR), see Methods). To determine whether the G members of a gene set S are enriched at the top of this list (step 3), a Kolmogorov-Smirnov (K-S) running sum statistic is computed: beginning with the top ranking gene, the running sum increases when a gene annotated to be a member of gene set S is encountered, and decreases otherwise. The enrichment score (ES) for a single gene set is defined as the greatest positive deviation of the running sum across all N genes. When many members of S appear at the top of the list, ES is high. The enrichment score is computed for every gene set using actual data, and the maximum ES (MES) achieved is recorded (step 4). To determine whether one or more of the gene sets are enriched in one diagnostic class relative to the other (step 5), the entire procedure (steps 2-4) is repeated 1000 times, using permuted diagnostic assignments, and building a histogram of the maximum ES achieved by any pathway in a given permutation. The MES achieved using the actual data is then compared to this histogram (step 6, red arrow), providing us with a global P-value for assessing whether any gene set is associated with the diagnostic categorization.

FIG. 2 shows that OXPHOS gene expression is reduced in diabetic muscle. (a) The mean expression of all genes (gray) and for OXPHOS genes (red) is plotted for DM2 vs. NGT individuals. (b) Histogram of mean gene expression level differences between NGT and DM2, using the data from (b), for all genes (black) and for OXPHOS genes (red).

FIG. 3 shows that OXPHOS-CR represents a co-regulated subset of OXPHOS genes responsive to the transcriptional co-activator PGC-1α. (a) Normalized expression profile of 52 mouse homologs of the human OXPHOS genes across the mouse expression atleas (Su, A. I. et al. Proc Natl Acad Sci USA 99, 4465-70. (2002)). These 52 genes were hierarchically clustered (Eisen et al. Proc Natl Acad Sci USA 95, 14863-8. (1998)). The purple tree corresponds to a sub-cluster with a correlation coefficient of 0.65. Applicants call the human homologs of these mouse genes the OXPHOS-CR set. The human homologs of this tightly coregulated cluster, marked with an * and delimited with a yellow box, are: ATP5J, ATP5L, ATP5O, COX5B, COX6A2, COX7A1, COX7B, COX7C, CYC1, CYCS, GRIM19, HSPC051, NDUFA2, NDUFA5, NDUFA7, NDUFA8, NDUFB3, NDUFB5, NDUFB6, NDUFC1, NDUFS2, NDUFS3, NDUFS5, SDHA, SDHB, UQCRB, UQCRC1. (b) Normalized expression profile of OXPHOS mouse homologs in a mouse skeletal muscle cell line during a three-day time course in response to PGC-1α. The expression profile includes infection with control (GFP) or with PGC-1α, at day 0 (prior to infection) as well as on days 1, 2, and 3 following adenoviral infection, all performed in duplicate.

FIG. 4 shows that OXPHOS-CR accounts for the bulk of OXPHOS signal seen in NGT vs. DM2. Histogram of signal:noise ratio for (a) All 10,983 human genes meeting the clipping and filtering criteria in the GSEA enrichment screen between NGT and DM2, (b) 106 OXPHOS genes meeting these clipping and filtering criteria, (c) 47 OXPHOS genes for which reliable mouse homologs are available in the mouse microarray, (d) OXPHOS-CR genes, and (e) OXPHOS genes but not in the OXPHOS-CR set.

FIG. 5 shows that OXPHOS-CR predicts total body aerobic capacity (VO2max). (a) Linear regression was used to model VO2max with diabetes status, the mean centroid of OXPHOS-CR gene expression, ubiquinol cytochrome c reductase binding protein (UQCRB) expression, or in combination as explanatory predictor) variables. The explanatory power and significance of the model are shown in the table. (b) Linear regression of VO2max against the mean centroid of OXPHOS-CR gene expression.

FIG. 6 shows previously known and newly identified mitochondrial proteins (mito-P). (A) Proteomic survey of mitochondria from mouse brain, heart, kidney, and liver resulted in the identification of 422 proteins, 262 of which were previously annotated as being mitochondrial. The distributions for (B) molecular weight, (C) isoelectric point, (D) mitochondrial compartments are plotted for proteins detected (pink) or not detected (blue) by our proteomic survey. Isolectric point, molecular weight, and subcellular distribution data came from the MITOchondria Project (MITOP, (Scharfe et al., 2000)). (E) Cumulative distribution of mRNA abundance for those genes whose protein product was detected (pink) or not detected (blue) by proteomics. The median expression levels for both groups are indicated. The cumulative distribution function for the proteins detected in proteomics is significantly greater than the cumulative distribution function for proteins not detected (Kolmogorov-Smirnov statistic, D=0.3618, P=9.4×10−18).

FIG. 7 shows modules of tightly co-regulated mito-P genes. Pairwise correlation matrix for the 388 mitochondrial genes present in the GNF mouse tissue compendium. Red represents strong positive correlation, blue represents strong negative correlation. Dominant gene modules are labeled 1-7 with functional annotations.

FIG. 8 shows the mRNA expression profile for 388 mitochondrial genes (rows) across 47 different mouse tissues (columns) in the GNF mouse expression atlas (Su et al., 2002). These genes and tissues were hierarchically clustered and visualized using DCHIP (Schadt et al., 2001). Key tissues showing high expression levels are labeled at the top of the panel. Evidence for being in mito-P is indicated by the white (previously known but not found in proteomics), gray (previously known and found in proteomics), and black (not previously known but found in proteomics) bars placed to the right of the correlogram.

FIG. 9 shows mitochondria neighborhood analysis. The mitochondria neighborhood index (N₁₀₀) is defined as the number of mito-P genes that occur within the nearest 100 expression neighbors of a given gene. The distribution of N₁₀₀ is plotted for all genes (white), mito-P genes (gray), and for the ancestral mito-P genes (black).

FIG. 10 shows a schematic overview of motifADE and application to the PGC-1a timecourse. (A) motifADE identifies motifs associated with differential expression. It begins with a list of genes ordered on the basis of differential expression across two conditions. Each gene is then annotated for the presence of a given motif in the promoter region. A nonparametric statistic is used to assess whether genes with the motif tend to rank high on this list (see Methods). In this example, genes with Motif 1 are randomly distributed on the list, while genes with Motif 2 tend to rank high, suggesting an association between Motif 2 and the differential expression. (B) C2C12 cells were infected with an adenovirus expressing either GFP (control) or with PGC-1α and profiled over a three day period. Experiments were performed in duplicate and relative gene expression measures are shown. Genes are ranked according to the difference in expression between PGC-1a and GFP on day 3. Mouse genes having a perfect Errα motif (5′-TGACCTTG-3′), a perfect Gabpa/b motif (5′-CTTCCG-3′), or both motifs are labeled with a black bar on the right side of the correlogram.

FIG. 11 shows a proposed model of mechanism of action of PGC-1a. PGC-1a is a highly regulated gene that responds to external stimuli, e.g., reduced in diabetes and increased following exercise. When PGC-1a levels rise, the expression of Errα and Gabpa are immediately induced via a double positive feedback loop. This results in the strong induction of Errα as well as Gabpa. These levels rise and over the course of 2-3 days, these factors couple with PGC-1a to induce the expression of NRF-1 as well as hundreds of downstream targets, such as OXPHOS and other mitochondrial genes.

FIG. 12 shows cooperativity between the Errα and Gabpa binding sites. All 5034 genes from motifADE analysis are rank ordered on the basis of expression difference (signal to noise ratio) on day 3 between cells treated with PGC-1a vs. GFP. The cumulative fraction of genes with a specified motif (Errα, blue; Gabpa, pink; both, black) is plotted as a function of fractional rank ordering of all 5034 genes.

### DETAILED DESCRIPTION OF THE INVENTION

I. Overview

The invention broadly relates to novel therapeutics for regulating metabolism, mitochondrial function, and for treating disorders, including obesity and type 2 diabetes, and to related methods. The invention stems, in part, from the discovery by applicants of a new group of coordinately-regulated genes, termed OXPHOS, which are involved in oxidative phosphorylation. OXPHOS-CR genes have the following key characteristics: (a) they are members of oxidative phosphorylation; (b) they are transcriptionally co-regulated and highly expressed at the major sites of insulin mediated glucose uptake (brown fat, heart, skeletal muscle); (c) they are targets of the transcriptional co-activator PPARGC1 (PGC-1α); (d) they show a subtle but extremely consistent expression decrease in diabetic and pre-diabetic muscle; and (e) their expression predicts total body aerobic capacity in humans.

Applicant have discovered that OXPHOS genes are downregulated in subjects afflicted with type 2 diabetes or with glucose intolerance and that Peroxisome Proliferator-Activated Receptor γ-Coactivator-1α (PGC-1α) transcriptionally regulates the OXPHOS genes. Applicants have also discovered that PGC-1α acts through Errα and Gabp to regulate OXPHOS gene expression. Such discoveries provide the basis for novel assays and methods of treatment relating to the genes and disorders.

The invention provides, in part, methods of modulating mitochondrial function, expression of the OXPHOS genes, mitochondrial biogenesis, expression of Nuclear Respiratory Factor 1 (NRF-1), β-oxidation of fatty acids, total mitochondrial respiration, uncoupled respiration, mitochondrial DNA replication, or expression of mitochondrial enzymes, by modulating the expression or activity of Errα, Gabpa, Gabpb or of genes containing Errα binding sites, Gabpa binding sites, or both. Modulation of these biological activities may be carried out in a cell, such as contacting a cell with an agent, or in a subject in need thereof. The invention further provides agents for treating these disorders and for modulating Errα, Gabp and PGC-1 function.

A related aspect of the invention provides a method of identifying agents useful for treating disorders related to altered glucose homeostasis, insulin resistance or reduced mitochondrial function. Furthermore, the invention provides methods of diagnosing such disorders or of identifying subjects at risk of developing the disorders.

The invention also provides cell-based methods of identifying agents which modulate the expression of OXPHOS genes. Since applicants have discovered that PGC-1α, Errα and Gabp regulate the expression of level of OXPHOS genes, such methods are useful in identifying agents which regulate the expression or activity of PGC-1α, Errα and Gabp. Furthermore, expression of OXPHOS genes may be used to predict total body aerobic capacity in humans and other mammals.

Another aspect of the invention provides a method of detecting statistically-significant differences in the expression level of at least one biomarker belonging to a biomarker set, between the members of a first and of a second experimental group. Such a method may be applied, for example, to identify biomarker sets which are differentially expressed in an experimental group afflicted with a disorder, even when the changes in expression between the two groups are very subtle. Biomarker sets identified using the methods described herein may be used in the development of diagnostic tools and treatments for the disorder for which they are associated. A related aspect of the invention provides methods of identifying transcriptional regulators which display differential activity between two sets of conditions. Such methods may be applied to the bio markers identified using the related methods provided herein, and may be useful in identifying disease genes and targets for novel therapeutics to treat or prevent disease.

II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The term “expression vector” and equivalent terms are used herein to mean a vector which is capable of inducing the expression of DNA that has been cloned into it after transformation into a host cell. The cloned DNA is usually placed under the control of (i.e., operably linked to) certain regulatory sequences such a promoters or enhancers. Promoters sequences maybe constitutive, inducible or repressible.

The term “operably linked” is used herein to mean molecular elements that are positioned in such a manner that enables them to carry out their normal functions. For example, a gene is operably linked to a promoter when its transcription is under the control, of the promoter and, if the gene encodes a protein, such transcription produces the protein normally encoded by the gene. For example, a binding site for a transcriptional regulator is said to be operably linked to a promoter when transcription from the promoter is regulated by protein(s) binding to the binding site. Likewise, two protein domains are said to be operably linked in a protein when both domains are able to perform their normal functions.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “promoter” is used herein to mean a DNA sequence that initiates the transcription of a gene. Promoters are typically found 5′ to the gene and located proximal to the start codon. If a promoter is of the inducible type, then the rate of transcription increases in response to an inducer. Promoters may be operably linked to DNA binding elements that serve as binding sites for transcriptional regulators. The term “mammalian promoter” is used herein to mean promoters that are active in mammalian cells. Similarly, “prokaryotic promoter” refers to promoters active in prokaryotic cells.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non-homologous recombination.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “microarray” refers to an array of distinct polynucleotides or oligonucleotides synthesized on a substrate, such as paper, nylon or other type of membrane, filter, chip, glass slide, or any other suitable solid support.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The terms “level of expression of a gene in a cell” or “gene expression level” refer to the level of mRNA, as well as pre-mRNA nascent transcript(s), transcript processing intermediates, mature mRNA(s) and degradation products, encoded by the gene in the cell.

The term “modulation” refers to upregulation (i.e., activation or stimulation), downregulation (i.e., inhibition or suppression) of a response, or the two in combination or apart. A “modulator” is a compound or molecule that modulates, and may be, e.g., an agonist, antagonist, activator, stimulator, suppressor, or inhibitor.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

The term “improving mitochondrial function” may refer to (a) substantially (e.g., in a statistically significant manner, and preferably in a manner that promotes a statistically significant improvement of a clinical parameter such as prognosis, clinical score or outcome) restoring to a normal level at least one indicator of glucose responsiveness in cells having reduced glucose responsiveness and reduced mitochondrial mass and/or impaired mitochondrial function; or (b) substantially (e.g., in a statistically significant manner, and preferably in a manner that promotes a statistically significant improvement of a clinical parameter such as prognosis, clinical score or outcome) restoring to a normal level, or increasing to a level above and beyond normal levels, at least one indicator of mitochondrial function in cells having impaired mitochondrial function or in cells having normal mitochondrial function, respectively. Improved or altered mitochondrial function may result from changes in extra-mitochondrial structures or events, as well as from mitochondrial structures or events, in direct interactions between mitochondrial and extra-mitochondrial genes and/or their gene products, or in structural or functional changes that occur as the result of interactions between intermediates that may be formed as the result of such interactions, including metabolites, catabolites, substrates, precursors, cofactors and the like.

The term “effective amount” refers to the amount of a therapeutic reagent that when administered to a subject by an appropriate dose and regime produces the desired result.

The term “subject in need of treatment for a disorder” is a subject diagnosed with that disorder or suspected of having that disorder.

The term “metabolic disorder” refers to a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin response). Examples of metabolic disorders include obesity, diabetes, hyperphagia, hypophagia, endocrine abnormalities, triglyceride storage disease, Bardet-Biedl syndrome, Lawrence-Moon syndrome, Prader-Labhart-Willi syndrome, Kearns-Sayre syndrome, anorexia, medium chain acyl-CoA dehydrogenase deficiency, and cachexia. Obesity is defined as a body mass index (BMI) of 30 kg/²m or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/²m or more, 26 kg/²m or more, 27 kg/²m or more, 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998)).

A “susceptibility locus” for a particular disease is a sequence or gene locus implicated in the initiation or progression of the disease. The susceptibility locus can be, for example, a gene or a microsatellite repeat, as identified by a microsatellite marker, or can be identified by a defined single nucleotide polymorphism. Generally, susceptibility genes implicated in specific diseases and their loci can be found in scientific publications, but may also be determined experimentally.

The term “Gabp polypeptide” comprises Gabpa and Gabpb polypeptides. In preferred embodiments of the methods described herein, the Gabpa and Gabpb polypeptides are mammalian polypeptides, preferably human. The amino acid sequences of human Gabpa and Gabpb are deposited as Genbank Accession Nos. NP_{—}002031 and NP_{—}852092, respectively. Gabpa is also known as E4TF1-53 in the art, while Gabpb is also known as E4TF1-60. Additional assays to those described herein for assaying the transcriptional activity of Gabpa and Gabpb, and additional isoforms of these subunits, may be found in the art (Sawa et al., Nucleic Acids Res. 24(24):4954-61 (1996); Watanabe, et al. Mol. Cell. Biol. 13 (3), 1385-1391 (1993), Sawada, J. et al J. Biol. Chem. 274 (50), 35475-35482 (1999); Suzuki, F. et al J. Biol. Chem. 273 (45), 29302-29308 (1998); Sawa, C., et al. Nucleic Acids Res. 24 (24), 4954-4961 (1996); Gugneja, S. et al Mol. Cell. Biol. 15 (1), 102-111 (1995); de la Brousse, F. C. et al. Genes Dev. 8 (15), 1853-1865 (1994); Virbasius, J. V. et al. Genes Dev. 7 (3), 380-392 (1993)), the teachings of which are incorporated by referenced herein.

The term “PGC-1 polypeptide” comprises PGC-1a and PGC-1b polypeptides. In preferred embodiments of the methods described herein, the PGC-1a and PGC-1b polypeptides are mammalian polypeptides, preferably human. The amino acid sequences of human PGC-1a and PGC-1b are deposited as Genbank Accession Nos. NP_{—}573570 and AF453324, respectively. Additional assays to those described herein for assaying the transcriptional activity of Gabpa and Gabpb, and additional isoforms of these subunits, may be found in the art (Huss, J. M., et al. Biol. Chem. 277 (43), 40265-40274 (2002); Kressler, D., et al. J. Biol. Chem. 277 (16), 13918-13925 (2002); Lin, J., et al. J. Biol. Chem. 277 (3), 1645-1648 (2002); Lin et al. J. Biol. Chem., Vol. 277, Issue 3, 1645-1648, Jan. 18, (2002)), the teachings of which are incorporated by referenced herein.

The term “Errα polypeptide” includes Errα polypeptides from any species. In some preferred embodiments of the methods described herein, an Errα polypeptide is a mammalian polypeptide, preferably a human polypeptide. The sequence of human Errα corresponds to Genbank Accession No. NP_{—}004442. Additional isoforms of Errα and methods for assaying Errα activity are known in the art e.g. Schreiber, S. N., et al. J. Biol. Chem. 278 (11), 9013-9018 (2003); Igarashi, M., et al. J. Gen. Virol. 84 (Pt 2), 319-327 (2003); Kraus, R. J., et al. J. Biol. Chem. 277 (27), 24826-24834 (2002); Vanacker, J. M., Oncogene 17 (19), 2429-2435 (1998); Sladek, R., et al. Genomics 45 (2), 320-326 (1997); Sladek, R., et al. Mol. Cell. Biol. 17 (9), 5400-5409 (1997); Shi, H., et al. Genomics 44 (1), 52-60 (1997); Yang, N., et al. J. Biol. Chem. 271 (10), 5795-5804 (1996); Giguere, V et al. Nature 331 (6151), 91-94 (1988); Eiler, S., et al Protein Expr. Purif. 22 (2), 165-173 (2001), the teachings of which are incorporated by referenced herein.

The term “nuclear hormone receptors” comprises comprise a large, well-defined family of ligand-activated transcription factors which modify the expression of target genes by binding to specific cis-acting sequences (Laudet et al., 1992, EMBO J, Vol, 1003-1013; Lopes da Silva et al., 1995, TINS 18, 542-548; Mangelsdorf et al., 1995, Cell 83, 835-839; Mangelsdorf et al., 1995, Cell 83, 841-850). Family members include both orphan receptors and receptors for a wide variety of clinically significant ligands including steroids, vitamin D, thyroid hormones, retinoic acid, etc. Additional receptors may be found in the literature (See for example The Nuclear Receptor FactsBook; Vincent Laudet (Editor); Elsevier Science & Technology, 2001).

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies.

The term “recombinant” as used in reference to a nucleic acid indicates any nucleic acid that is positioned adjacent to one or more nucleic acid sequences that it is not found adjacent to in nature. A recombinant nucleic acid may be generated in vitro, for example by using the methods of molecular biology, or in vivo, for example by insertion of a nucleic acid at a novel chromosomal location by homologous or non-homologous recombination. The term “recombinant” as used in reference to a polypeptide indicates any polypeptide that is produced by expression and translation of a recombinant nucleic acid.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a fall length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local identity algorithm (Smith and Waterman, Adv. Appl. Math., 2:482 (1981)), by the identity alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of identity over the comparison window) generated by the various methods is selected.

The term “diagnostic” refers to assays that provide results which can be used by one skilled in the art, typically in combination with results from other assays, to determine if an individual is suffering from a disease or disorder of interest such as diabetes, including type I and type II, whereas the term “prognostic” refers to the use of such assays to evaluate the response of an individual having such a disease or disorder to therapeutic or prophylactic treatment. The term “pharmacogenetic” refers to the use of assays to predict which individual patients in a group will best respond to a particular therapeutic or prophylactic composition or treatment.

Other technical terms used herein have their ordinary meaning in the art that they are used, as exemplified by a variety of technical dictionaries, such as the McGraw-Hill Dictionary of Chemical Terms and the Stedman's Medical Dictionary.

III. Methods of Modulating Biological Responses in a Cell

In one aspect, the invention provides methods of modulating biological responses in a cell. One specific aspect of the invention provides a method of modulating a biological response in a cell, the method comprising contacting the cell with at least one agent that modulates the expression or activity of Errα or Gabp, wherein the biological response is (a) expression of at least one OXPHOS gene; (b) mitochondrial biogenesis; (c) expression of Nuclear Respiratory Factor 1 (NRF-1); (d) β-oxidation of fatty acids; (e) total mitochondrial respiration; (f) uncoupled respiration; (g) mitochondrial DNA replication; (h) expression of mitochondrial enzymes; or (i) skeletal muscle fiber-type switching.

In one embodiment of the methods described herein, the biological response that is modulated is the expression of at least one OXPHOS gene. OXPHOS genes have been described in Mootha et al., Nat. Genet. 2003; 34(3):267-73, hereby incorporated by reference in its entirety. In one embodiment, the OXPHOS gene is NDUFB3, SDHA, NDUFA8, COX7A1, UQCRC1, NDUFC1, NDUFS2, ATP5O, NDUFS3, SDHB, NDUFS5, NDUFB6, COX5B, CYC1, NDUFA7, UQCRB, COX7B, ATP5L, COX7C, NDUFA5, GRIM19, ATP5J, COX6A2 NDUFB5, CYCS, NDUFA2 or HSPC051.

In another embodiment of the methods described herein, the biological response that is modulated is mitochondrial biogenesis. U.S. Patent Publication No. 2002/0049176 describes assays for determining mitochondrial mass, volume or number, and is hereby incorporated by reference in its entirety.

In another embodiment of the methods described herein, the biological response that is modulated is expression of Nuclear Respiratory Factor 1 (NRF-1). NRF-1 is a transcription factor occurring as a homodimer of a 54 KDa polypeptide encoded by the nuclear gene nrf-1 (Evans and Scarpulla, Genes & Development 4:1023-1034 (1990), Scarpulla, J. Bioenergetics and Biomembranes 29:109-119 (1997), Moyes et al., J. Exper. Biol. 201:299-307 (1998)). NRF-1 binds to the upstream promoters of nuclear genes that encode respiratory components associated with mitochondrial transcription and replication. NRF-1 can be any NRF-1, such as rat, mouse or human. NRF-1 nucleotide and polypeptide sequences are described in U.S. Patent Publication No. 20020049176, hereby incorporated by reference in its entirety.

In another embodiment of the methods described herein, the biological response that is modulated is β-oxidation of fatty acids. In another embodiment of the methods described herein, the biological response that is modulated is total mitochondrial respiration. In another embodiment of the methods described herein, the biological response that is modulated uncoupled respiration. Uncoupled respiration occurs when electron transport is uncoupled from ATP synthesis

In another embodiment of the methods described herein, the biological response that is modulated is mitochondrial DNA replication. Quantification of mitochondrial DNA (mtDNA) content may be accomplished by one with routine skill in the art using any of a variety of established techniques that are useful for this purpose, including but not limited to, oligonucleotide probe hybridization or polymerase chain reaction (PCR) using oligonucleotide primers specific for mitochondrial DNA sequences (see, e.g., Miller et al., 1996 J. Neurochem. 67:1897; Fahy et al., 1997 Nucl. Ac. Res. 25:3102; U.S. patent application Ser. No. 09/098,079; Lee et al., 1998 Diabetes Res. Clin. Practice 42:161; Lee et al., 1997 Diabetes 46(suppl. 1): 175A). A particularly useful method is the primer extension assay disclosed by Fahy et al. (Nucl. Acids Res. 25:3102, 1997) and by Ghosh et al. (Am. J. Hum. Genet. 58:325, 1996). Suitable hybridization conditions may be found in the cited references or may be varied according to the particular nucleic acid target and oligonucleotide probe selected, using methodologies well known to those having ordinary skill in the art (see, e.g., Ausubel et al., Current Protocols in Molecular Biology, Greene Publishing, 1987; Sambrook et al., Molecular Cloning: A Laboratory Manual, Cold Spring Harbor Press, 1989).

In another embodiment of the methods described herein, the biological response that is modulated is expression of mitochondrial enzymes. In one embodiment, mitochondrial enzymes are Electron Transport Chain (ETC) enzymes. An ETC enzyme refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths. Enzymol. 260:14; Ernster et al., 1981 J. Cell Biol. 91:227s-255s, and references cited therein). A mitochondrial enzyme of the present invention may also comprise a Krebs cycle enzyme, which includes mitochondrial molecular components that mediate the series of biochemical/bioenergetic reactions also known as the citric acid cycle or the tricarboxylic acid cycle (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the α-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide and nucleoside diphosphokinase.

In another embodiment of the methods described herein, the biological response that is modulated is skeletal muscle fiber-type switching, that is, a shift towards type I oxidative skeletal muscle fibers. International PCT Application WO 03/068944 describes skeletal muscle fiber-type switching. In some embodiments, the agent increases at least one of the biological responses. In alternate embodiments, the agent decreases at least one of the biological responses.

The methods described herein for modulating a biological activity in a cell may be applied to any type of cell. In specific embodiments, the cell is a skeletal muscle cell, a smooth muscle cell, a cardiac muscle cell, a hepatocyte, an adipocyte, a neuronal cell, or a pancreatic cell. The cell may be a primary cell, a cell derived from a cell line, or a cell which has differentiated in vitro, such as a differentiated cell obtained through manipulation of a stem cell. In some embodiments, the cell in an organism, while in other embodiments the cell is manipulated ex vivo, such as in cell or tissue culture. The methods described herein also apply to groups of cells, such as to whole tissues or organs. In some embodiments, the organism is a mammal, such as a mouse, rat, an ungulate, a horse, a dog or a human.

In some embodiments, the human is afflicted, at risk of developing, or suspected with being afflicted, with a disorder. In some embodiments, the disorder comprises a metabolic disorder, a disorder characterized by altered mitochondrial activity, a disorder characterized by sugar intolerance, or a combination thereof. In specific embodiments of the methods described herein, the disorder is diabetes, obesity, cardiac myopathy, aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRY), NARP (Neuropathy; Ataxia; Retinitis Pigmentosa), MNGIE (Myopathy and external ophthalmoplegia, neuropathy; gastrointestinal encephalopathy, Kearns-Sayre disease, Pearson's Syndrome, PEO (Progressive External Ophthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, and mitochondrial diabetes and deafness (MIDD), mitochondrial DNA depletion syndrome.

In one embodiment of the methods for modulating biological responses in a cell described herein, the agent modulates the formation of a complex between a PGC-1 polypeptide and (i) an Errα polypeptide; or (ii) a Gabp polypeptide. The agent may be an agent which increases formation of the complex in the cell, or it may be an agent that reduces formation of the complex in the cell. In embodiments where the agent increases a biological activity of the cell, the agent increases complex formation, whereas in embodiments where a biological activity is to be decreased, complex formation is decreased. One skilled in the art would recognize that complex formation, as used herein, refers to the normal association between the polypeptides which results in the transcriptional activation of target genes by the complex. Therefore, an agent which resulted in an aberrant aggregation of PGC-1α and Errα polypeptides, wherein the resulting complex has reduced transcriptional activating activity, would not result in increased biological activity but instead in less. Likewise, an agent which increased complexed formation, but the resulting complex was degraded in the cell, would result in less biological activity in the cell. Accordingly, in some specific embodiments for reducing biological activity, the agent results in increase complex formation, wherein the complex has reduced transcriptional activity or stability.

In one embodiment of the methods for modulating biological responses in a cell described herein, the agent modulates the expression level or the transcriptional activity of an Errα polypeptide, a Gabp polypeptide, or of both. The agent may comprise a polypeptide, a nucleic acid, or a chemical compound. In one embodiment of the methods for modulating biological responses in a cell described herein, the agent is itself an Errα polypeptide or fragments thereof, or a Gapb polypeptide or a fragment thereof, or a nucleic acid encoding such polypeptides or fragments thereof.

In some embodiments of the methods for increasing biological responses in a cell described herein, the agent increases complex formation between a PGC-1 polypeptide and an Errα polypeptide. In preferred embodiments, the agent is specific for the complex formation between a PGC-1 polypeptide and an Errα polypeptide. In a preferred embodiment, the agent increases Errα activity by preferentially promoting complex formation between a PGC-1 polypeptide and an Errα polypeptide over complex formation between a PGC-1 polypeptide and at least one other polypeptide to which PGC-1 normally binds in an organism. Polypeptides to which PGC-1 normally binds in an organism include the following: nearly all nuclear receptor (e.g., PPAR-gamma, PPAR-alpha, thyroid hormone receptor, HNF4α, etc.) as well as other transcription factors, such as NRF1, NFAT, etc (see Puigserver and Spiegelman, Endocr Rev. 2003; 24(1):78-90).

In another preferred embodiment, the agent increases Errα activity by preferentially promoting complex formation between a PGC-1 polypeptide and an Errs polypeptide over a PGC-1 polypeptide and another nuclear receptor. In some embodiments, the affinity of an agent which increases complex formation between PGC-1 polypeptide and Errα does so at least 2, 5, 10, 20, 40, 50, 100, 200, 500, 1000, 5000, 10,000, 50,000 or 100,000-fold times more potently than complex formation between the same PGC-1 polypeptide and (i) at least another polypeptide to which PGC-1 normally binds in an organism; or (ii) a nuclear receptor; or (iii) both. The fold-level of potency may be determined by measuring the association constant, the disassociation constant, or more preferably the K_{d} of the agent for the various complexes.

In parallel embodiments of the methods for inhibiting a biological response in a cell described herein, the agent preferentially inhibits complex formation between a PGC-1 polypeptide and an Errα polypeptide over a PGC-1 polypeptide and another nuclear receptor. In some embodiments, the affinity of an agent which decreases complex formation between PGC-1 polypeptide and an Errα does so at least 2, 5, 10, 20, 40, 50, 100, 200, 500, 1000, 5000, 10,000, 50,000 or 100,000-fold times more potently than complex formation between the same PGC-1 polypeptide and (i) at least another polypeptide to which PGC-1 normally binds in an organism; or (ii) a nuclear receptor; or (iii) both. In other embodiments, the IC50 for disrupting the interaction between a PGC-1 polypeptide and an Errα polypeptide is 2, 5, 10, 20, 40, 50, 100, 200, 500, 1000, 5000, 10,000, 50,000 or 100,000-fold lower than that for disrupting the interaction between a PGC-1 polypeptide and (i) at least one another polypeptide to which PGC-1 normally binds in an organism; or (ii) a nuclear hormone receptor.

In other embodiments of the methods described herein for modulating biological responses in a cell, a Gabp polypeptide may replace the Errα polypeptide. For example, instead of using an agent that modulates the interaction between a PGC-1 polypeptide and an Errα polypeptide, an agent is used that modulates the interaction between a polypeptide PGC-1 polypeptide and an Gabp polypeptide. Thus all variations of the methods described herein for modulating biological responses in a cell using an Errα polypeptide may be applied to an Gabp polypeptide, such as a Gabpa polypeptide.

Another embodiment of the methods described herein for modulating biological responses in a cell, the cell is contacted with two agents, wherein one agent modulates the expression or activity of Errα and the other agent modulates the expression or activity of a Gabp polypeptide, such as a Gabpa polypeptide. In another embodiment, the cell is contacted with one agent which modulates the expression or activity of both Errs and of a Gabp polypeptide.

IV. Methods of Preventing/Treating Disease

Some aspects of the invention provide methods of treating or preventing a disorder. Some aspects provide methods of preventing disorders which are associated with glucose intolerance, excess glucose production, insulin resistance, aberrant metabolism or abnormal mitochondrial function.

The invention further provides agents for the manufacture of medicaments to treat any of the disorders described herein. Any methods disclosed herein for treating or preventing a disorder by administering an agent to a subject may be applied to the use of the agent in the manufacture of a medicament to treat that disorder. For example, in one specific embodiment, an Errα agonist may be used in the manufacture of a medicament for the treatment of a disorder characterized by low mitochondrial function or by sugar intolerance, such as diabetes.

One aspect of the invention provides method of treating or preventing a disorder characterized by reduced mitochondrial function, glucose intolerance, or insulin intolerance in a subject, the method comprising administering to the subject a therapeutically effective amount of an agent which (i) increases the expression or activity of Errα or Gabp or both; or (ii) increases the formation of a complex between a PGC-1 polypeptide and (a) an Errα polypeptide; (b) a Gabp polypeptide; or both; or (iii) binds to an (a) Errα binding site, or to a (b) Gabpa binding site, and which increases transcription of at least one gene in the subject, said gene having an Errα binding site, a Gabpa binding site, or both.

In one embodiment, the agent which binds to an (a) Errα binding site, or to a (b) Gabp binding site, comprises at least one DNA binding domain. In a further embodiment, the DNA binding domain comprises at least one zinc-finger. In some embodiments, such agents comprise a DNA binding domain and a transactivation domain. Methods are known in the art for designing transcriptional activator or repressors which bind to specific DNA sequences, including those disclosed in U.S. Pat. Nos. 6,607,882, 6,453,242 and 6,511,808.

In one embodiment, the disorder is type 2 diabetes mellitus. In one embodiment of any of the methods described herein, a disorder characterized by reduced mitochondrial function, glucose intolerance, or insulin intolerance is diabetes, obesity, cardiac myopathy, aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRF), NARP (Neuropathy; Ataxia; Retinitis Pigmentosa), MNGIE (Myopathy and external ophthalmoplegia, neuropathy; gastro-intestinal encephalopathy, Kearns-Sayre disease, Pearson's Syndrome, PEO (Progressive External Ophthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, and mitochondrial diabetes and deafness (MIDD), mitochondrial DNA depletion syndrome.

The invention further provides a method of treating or preventing a disorder characterized by reduced mitochondrial function, glucose intolerance, or insulin intolerance in a subject, the method comprising administering to the subject a therapeutically effective amount of an agent which increases the expression or activity of a gene, wherein the gene has an Errα binding site or a Gapba binding site.

In one preferred embodiment of this method, the gene has both an Errα binding site and a Gapba binding site. In one embodiment, the Errα binding site comprises the sequence 5′-TGACCTTG-3′ or the sequence ′5-CAAGGTCA-3′. In one embodiment, the Gapba binding site comprises the sequence ′5-CTTCCG-3′ or ′5-CGGAAG-3′. It is well known by one of routine skill in the art that transcriptional factors may have optimal binding sites to which they may bind in vivo or in vitro with substantially the same binding affinity as their optimal binding sites. Accordingly, in some embodiments, an Errα binding site comprises any sequence that, when operably bound to a promoter, allows transcriptional control of the promoter by Errα. In another embodiment, an Errα binding site comprises any sequence that may be bound by an Errα polypeptide with high affinity, such as with a K_{d} that is less than at least about 10⁻⁵ M, about 10⁻⁶ M, about 10⁻⁷ M, about 10⁻⁸ M, about 10⁻⁹ M, about 10⁻¹⁰ M, about 10⁻¹¹ M, or about 10⁻¹² M. Likewise, in some embodiments, an Gabpa binding site comprises any sequence that, when operably bound to a promoter, allows transcriptional control of the promoter by Gabpa. In another embodiment, an Errα binding site comprises any sequence that may be bound by an Gabpa polypeptide with high affinity, such as with a K_{d} that is less than at least about 10⁻⁵ M, about 10⁻⁶ M, about 10⁻⁷ M, about 10⁻⁸ M, about 10⁻⁹ M, about 10⁻¹⁰ M, about 10⁻¹¹ M, or about 10⁻¹² M. In some embodiments, an Errα binding site comprises a sequence which is about 50%, 62.5%, 75%, or 87.5% identical to either 5′-TGACCTTG-3′ or to ′5-CAAGGTCA-3′. In some embodiments, a Gabpa binding site comprises a sequence which is about 50%, 66.6%, or 83.3%, identical to either ′5-CTTCCG-3′ or ′5-CGGAAG-3′.

In another embodiment of any of the methods described herein, a gene which has an Errα binding site is any one of the genes listed on Table 10, a gene which has a Gabpa binding site is any one of the genes on Table 11, and a gene having both an Errα and a Gabpa binding site is any one of the genes listed on Table 12.

In yet another embodiment of this method, the binding sites are located within the promoter region of the gene. In one embodiment, the promoter region comprises from at least 0.5, 1, 1.5, 2, 2.5, 3, 4, 5 or 10 kb upstream of the transcriptional start site of the gene to at least either (i) 0.5, 1, 1.5, 2, 2.5, 3, 4, 5 or 10 kb downstream of the transcriptional start site of the gene; or (ii) 0.5, 1, 1.5, 2, 2.5, 3, 4, 5 or 10 kb downstream of the stop codon of the gene. In yet another embodiment of this methods, the promoter region comprises a masked promoter region. A masked promoter region comprises the regions of promoters that are conserved between two organisms. For example, a masked promoter region may comprise the promoter sequences which are conserved between human and another mammal, such as a mouse. By sequences that are conserved, it is meant sequences which share at least 70% sequence identity between the two species across a window size of at least 8, 9, 11, 12, 13, 14, 15, 16, 17, 18, 19, 20, 25, or 50 nucleotides, or more preferably a window of 10 nucleotides.

In another embodiment, the binding sites are located within the promoter region, the coding region, the exons, the introns, or the untranslated region of the gene, or a combination thereof.

In yet another specific embodiment of the method, the gene having an Errα binding site or a Gapba binding site is not Errα, while in another embodiment, the gene is not Gabpa. The agent which increases the activity or expression of a specific gene may be selected by one skilled in the art according to the type of protein that is encoded. For example, if the gene encodes an enzyme, then enzyme activators are expected to increase the activity of the enzyme. Likewise, if the gene is a receptor, a receptor agonist may be administered. Such agonist may comprise small organic molecules, such as those having less than 1 kDa in mass, or may comprise an antibody that binds to the gene product and increases its activity. For any gene, an agent which increases the activity of the gene may comprise a polypeptide of the gene itself, or a nucleic acid containing the gene or an active fragment thereof.

In one embodiments of the methods described herein, reduced mitochondrial function comprises reduced total mitochondrial respiration, reduced uncoupled respiration, reduced expression of mitochondrial enzymes, reduced mitochondrial biogenesis or a combination thereof. In some embodiments of the methods for preventing or treating a disorder in a subject, at least one of the agents increases the expression or activity of Errα, of a Gabp polypeptide, or of both. In another embodiment, the agent promotes the expression or activity of a binding partner of PGC-1α or of PGC-1β. In yet another embodiment, the agent promotes the binding of PGC-1α to a transcriptional regulator. In some embodiments, the transcriptional regulator is Errα or Gabpa. In one preferred embodiment, the agent induces mitochondrial activity in skeletal muscle.

Another aspect of the invention provides a method of treating impaired glucose tolerance in an individual, comprising administering to the individual a therapeutically effective amount of an agent which increases the expression level of at least two OXPHOS-CR genes, thereby treating impaired glucose tolerance in the individual. Another aspect of the invention provides a method of treating obesity in an individual, comprising administering to the individual a therapeutically effective amount of an agent which increases the expression level of at least two OSPHOS-CR genes, thereby treating obesity in the individual. In preferred embodiments, the expression level of the OXPHOS-CR genes is increased in the skeletal muscle cells of the subject by at least 10%, 20%, 30%, 40%, 50% or 75%.

Another aspect of the invention provides methods of treating or preventing disorders characterized by an elevated metabolic rate in a subject and methods of lowering a metabolic rate in a subject. The invention provides a method of reducing the metabolic rate of a subject in need thereof, the method comprising administering to the subject a therapeutically effective amount of an agent which decreases the expression or activity of at least one of the following: (i) Errα; (ii) Gabpa; (iii) a gene having an Errα binding site, a Gabpa binding site, or both; or (iv) a transcriptional activator which binds to an Errα binding site or to a Gabpa binding site; thereby reducing the metabolic rate of the patient.

In some embodiments of the methods provided for reducing the metabolic rate of a subject in need thereof, the subject is afflicted with an infection, such as a viral infection. In one specific embodiment, the viral infection is a human immunodeficiency virus infection.

In another embodiment of methods for reducing metabolic rates, the subject is afflicted with cancer or with cachexia. Cachexia is a metabolic condition characterized by weight loss and muscle wasting. It is associated with a wide range of conditions including inflammation, heart failure and malignancies, and is well known and described in the clinical literature e.g., J. Natl. Cancer Inst. 89(23): 1763-1773 (1997) 1. The mechanistic derangements underlying cachexia are not known, but it is clear that a negative energy balance obtains in the face of severe weight loss. In specific embodiments, the subject is afflicted with cancer cachexia, pulmonary cachexia, Russell's Diencephalic Cachexia, cardiac cachexia or chronic renal insufficiency.

In some embodiments of the methods provided for reducing the metabolic rate of a subject in need thereof, the agent decreases the formation of a complex between a PGC-1 polypeptide and (i) an Errα polypeptide; or (ii) a Gabp polypeptide. In preferred embodiments, the PGC-1 polypeptide is a PGC-1α polypeptide. In another embodiment, the agent decreases the expression level or the transcriptional activity of an Errα polypeptide, a Gabp polypeptide, or of both, while in additional embodiments the agent inhibits the expression or activity of a gene which has an Errα binding site, a Gabpa binding site, or both. In some embodiments, the agents comprise double stranded RNA reagents, dominant negative polypeptides or nucleic acids encoding them, or antibodies directed to Errα, Gabpa, Gabpb, or to genes (or their gene products) which have an Errα binding site, a Gabpa binding site, or both, such as binding sites in their promoter regions.

U.S. Pat. No. 5,602,009 describes a method of generating inhibitory nuclear hormone receptors. Such methods may be applied to Errα or to Gabp to generate polypeptides or nucleic acids which encode them, which may be used as agents in the methods described herein for reducing the metabolic rate of a subject.

V. Methods of Diagnosing/Identifying Disease Genes

One aspect of the invention provides methods of identifying a susceptibility loci for a disorder characterized by reduced mitochondrial function or reduced metabolism. The identification of these loci allows for the diagnosis of the disorders and for the design or screening of agents for the treatment of these disorders.

The invention provides a method of identifying a susceptibility locus for a disorder that is characterized by reduced mitochondrial function, glucose intolerance, or insulin intolerance in a subject, the method comprising (i) identifying at least one polymorphisms in a gene, or linked to a gene, wherein the gene (a) has an Errα binding site, a Gabpa binding site, or both; or (b) is Errα, Gabpa, or Gabpb; (ii) determining if at least one polymorphism is associated with the incidence of the disorder, wherein if a polymorphism is associated with the incidence of the disorder then the gene having the polymorphism, or the gene to which the polymorphism is linked, is a susceptibility locus.

In one embodiment of the methods described herein for identifying a susceptibility locus for a disorder, the gene is any one of the gene listed on Tables 10-12.

As used herein, the term “polymorphism” refers to the co-existence, within a population, of more than one form of a gene or portion thereof (e.g. allelic variant), at a frequency too high to be explained by recurrent mutation alone. A portion of a gene of which there are at least two different forms, i.e. two different nucleotide sequences, is referred to as a polymorphic region of a gene”. A specific genetic sequence at a polymorphic region of a gene is an allele.

A polymorphic region can be a single nucleotide or more than one nucleotide, the identity of which differs in different alleles. A polymorphic region can be a restriction fragment length polymorphism (RFLP). A RFLP refers to a variation in DNA sequence that alters the length of a restriction fragment as described in Botstein et al., Am. J. Hum. Genet. 32. 3 14-33 1 (1980). The RFLP may create or delete a restriction site, thus changing the length of the restriction fragment. RFLPs have been widely used in human and animal genetic analyses (see WO 90/13668; WO90/11369; Donis-Keller, Cell 5 1, 3) 19-33)7 (1987); Lander et al. Genetics 121, 85-99 (1989)). When a heritable trait can be linked to a particular RFLP, the presence of the RFLP in an individual can be used to predict the likelihood that the individual will also exhibit the trait.

Other polymorphisms take the form of short tandem repeats (STRs) that include tandem di-, tri- and tetranucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307, 1 3-1 15 (1992); Horn et al. WO 91/14003; Jeffreys, EP 370,719), and in a large number of genetic mapping studies.

Other polymorphisms take the form of single nucleotide variations between individuals of the same species. Such single nucleotide variations may arise due to substitution of one nucleotide for another at the polymorphic site or from a deletion of a nucleotide or an insertion of a nucleotide relative to a referenced allele. These single nucleotide variations are referred to herein as single nucleotide polymorphism (SNPs). Such SNPs are far more frequent than RFLPS, STRs and VNTRs. Some SNPs may occur in protein-coding sequences, in which case, one of the polymorphic forms may give rise to the expression of a defective protein and, potentially, a genetic disease. Other SNPs may occur in noncoding regions. Some of these polymorphisms may also result in defective protein expression (e.g. as a result of defective splicing). Other SNPs may have no phenotypic effects.

Techniques for determining the presence of particular alleles would be those known to persons skilled in the art and include, but are not limited to, nucleic acid techniques based on size or sequence, such as restriction fragment length polymorphism (RFLP), nucleic acid sequencing, or nucleic acid hybridization. The nucleic acid tested may be RNA or DNA. These techniques may also comprise the step of amplifying the nucleic acid before analysis. Amplification techniques are known to those of skill in the art and include, but are not limited to, cloning, polymerase chain reaction (CR), polymerase chain reaction of specific alleles (PASA), polymerase chain ligation, nested polymerase chain reaction, and the like. Amplification products may be assayed in a variety of ways, including size analysis, restriction digestion followed by size analysis, detecting specific tagged oligonucleotide primers in the reaction products, allele-specific oligonucleotide (ASO) hybridization, allele specific exonuclease detection, sequencing, hybridization and the like. Polymorphic variations leading to altered protein sequences or structures may also be detected by analysis of the protein itself. Additional methods for the detection of polymorphisms are described in U.S. Pat. No. 6,453,244 and in International PCT publications No. WO 04/011668, WO 03/048384, WO 01/20031 and WO 03/038125, the teachings of which are hereby incorporated by reference.

General methods are available to one skilled in the art for determining if a particular allele is associated with the incidence of the disorder, such as those described in Analysis of Human Genetic Linkage, by Jurg Ott: Johns Hopkins University Press, 1999; and Statistical Genomics: Linkage, Mapping, and QTL Analysis by Ben Hui Liu: CRC Press, 1997.

The invention also provides a related method for determining if a subject is at risk of developing a disorder which is characterized by reduced mitochondrial function, the method comprising determining if a gene from the subject contains a mutation which reduces the function of the gene, wherein the gene has an Errα binding site, a Gapba binding site, or both, wherein if a gene from the subject contains a mutation then the subject is at risk of developing the disorder.

In one embodiment of this method, the mutation reduces the function of the gene. In another embodiment, the disorder is diabetes, obesity, premature aging, cardiomyopathy, a neurodegenerative disease, or retinal degeneration. In further embodiments, the gene is any one of the genes on Tables 10-12.

The proposed role of the candidate genes proteins can be validated by traditional overexpression or knockout approaches to ascertain the effects of such manipulations on mitochondrial biogenesis in the engineered cell lines. This approach ultimately identifies additional molecules whose expression or activity can be modulated to enhance mitochondrial function. For example, cultured skeletal muscle cells may be used with electrical stimulation or thyroid hormone as the stimulus for mitochondrial biogenesis. Alternatively, a fat cell culture such as 3T3-L1 cells may be used, with norepinephrine providing the stimulus for mitochondrial biogenesis. Alternatively, cultured cells such as HeLa or HEK293 that express PGC-1 and/or NRF-1 under a tetracycline inducible system may be used, wherein induced expression of PGC-1 and/or NRF-1 stimulates mitochondrial biogenesis. After sufficient time with the appropriate stimulus to allow induction (1-2 days), the cells are incubated with P³² orthophosphate for 4 hrs. Cells are then harvested and subjected to SDS-PAGE to resolve the labeled proteins. Using these systems, the function of a candidate disease gene may be altered, such as through overexpression, expression of dominant negative forms of the proteins, inhibitory RNAi reagents, antibodies, and the like, and the effects on mitochondrial biogenesis or function determined.

VI. Methods of Identifying Therapeutic Agents

One aspect of the invention provides methods of identifying agents which modulate biological responses in a cell, which modulate expression of the OXPHOS-CR genes or which prevent or treat a disorder.

One aspect of the invention provides a method of determining if an agent is a potential agent for the treatment of a disorder that is characterized by glucose intolerance, insulin resistance or reduced mitochondrial function, the method comprising determining if the agent increases: (i) the expression or activity of Errα or Gabp in a cell; or (ii) the formation of a complex between a PGC-1 polypeptide and (i) an Errα polypeptide; or (ii) a Gabp polypeptide; wherein an agent that increases (i) or (ii) is a potential target for the treatment of the disorder.

In some embodiments of the methods described herein for determining if an agent is a potential agent for the treatment of a disorder, the disorder is diabetes, obesity, cardiac myopathy, aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRF), NARP (Neuropathy; Ataxia; Retinitis Pigmentosa), MNGIE (Myopathy and external ophthalmoplegia, neuropathy; gastrointestinal encephalopathy, Kearns-Sayre disease, Pearson's Syndrome, PEO (Progressive External Ophthalmoplegia), congenital muscular dystrophy with mitochondrial structural abnormalities, Wolfram syndrome, Diabetes Insipidus, Diabetes Mellitus, Optic Atrophy Deafness; Leigh's Syndrome, fatal infantile myopathy with severe mitochondrial DNA (mtDNA) depletion, benign “later-onset” myopathy with moderate reduction in mtDNA, medium chain acyl-CoA dehydrogenase deficiency, dystonia, arthritis, and mitochondrial diabetes and deafness (MIDD) or mitochondrial DNA depletion.

Any general method known to one skilled in the art may be applied to determine if an agent increases the expression or activity of Errα or Gabp. In one specific embodiment for determining if an agent increases the expression of Errα or Gabp, a cell is contacted with an agent, and an indicator of gene expression, such as mRNA level or protein level, is determined. Levels of mRNA may be determined, for example, using such techniques as Northern Blots, reverse-transcriptase polymerase chain reaction (RT-PCR), RNA protection assays or a DNA microarray comprising probes capable of detecting Errα or Gabp mRNA or cDNA molecules. Likewise, protein levels may be quantitated using techniques well-known in the art, such as western blotting, immuno-sandwich assays, ELISA assays, or any other immunological technique. Techniques for quantitating nucleic acids and proteins may be found, for example, in Molecular Cloning: A Laboratory Manual, 3rd Ed., ed. by Sambrook and Russell (Cold Spring Harbor Laboratory Press: 2001); and in Current Protocols in Cell Biology, ed. by Bonifacino, Dasso, Lippincott-Schwartz, Harford, and Yamada, John Wiley and Sons, Inc., New York, 1999, hereby incorporated by reference in their entirety.

In one example, an RC cell culture system can be used to identify compounds which activate production of ERRα or, once ERRα production has been activated in the cells, can be used to identify compounds which lead to suppression or switching off of ERRα, production. Alternatively, such a cell culture system can be used to identify compounds or binding partners of ERRα which increase its expression. Compounds thus identified are useful as therapeutics in conditions where ERRα production is deficient or excessive. Similar experiments may be carried out with Gabpa or Gabpb or both.

Likewise, any general method known to one skilled in the art may be applied to determining if an agent increases the activity of Errα or Gabp. Activities of Errα or Gabp include their ability to bind to DNA, their ability to bind to other transcriptional regulators or their ability to promote transcription of target genes. In one embodiment, candidate agents are tested for their ability to modulate ERRα activity by (a) providing a system for measuring a biological activity of ERRα; and (b) measuring the biological activity of ERRα in the presence or absence of the candidate compound, wherein a change in ERRα activity in the presence of the compound relative to ERRα activity in the absence of the compound indicates an ability to modulate ERRα activity. In specific embodiments, the biological activity is the ability of Errα to bind the promoter of a target gene, such as the promoter or medium chain acyl-CoA dehydrogenase (MCAD), which may be determined using chromatin immunoprecipitation and analysis of the DNA bound to the Errα polypeptide. In another embodiment, the biological activity is the ability of Errα to complex with PGC-1a or PGC-1b, which may be measured by immunoprecipitation of either Errα or a PGC-1 polypeptide and determining the presence of the other protein by western blotting. In another embodiment, the biological activity is promoting transcription of a target gene. An indicator of gene expression for a target gene whose transcription is regulated by Errα or by Gabp can be compared between cells which have or have not been contacted with the agent. In specific embodiments, PGC-1α or PGC-1β is also present when testing of an agent modulates the transcriptional activating activity of Errα or Gabp polypeptides. Target genes which may be used include those which contain either an Errα or a Gabp binding site, such as OXPHOS genes or those provided by the invention. Because Gabpa and Gabpb form a complex, in some preferred embodiments both proteins, or nucleic acids encoding them, are present in the assay systems described herein.

One particular embodiment for identifying agents which modulate activity of Errα employs two genetic constructs. One is typically a plasmid that continuously expresses the transcriptional regulator of interest when transfected into an appropriate cell line. The second is a plasmid which expresses a reporter, e.g., luciferase under control of the transcriptional regulator. For example, if a compound which acts as a ligand for Errα is to be evaluated, one of the plasmids would be a construct that results in expression of the Errα in the cell line. The second would possess a promoter linked to the luciferase gene in which an Errα response element is inserted. If the compound to be tested is an agonist for the Errα receptor, the ligand will complex with the receptor and the resulting complex binds the response element and initiates transcription of the luciferase gene. In time the cells are lysed and a substrate for luciferase added. The resulting chemiluminescence is measured photometrically. Dose response curves are obtained and can be compared to the activity of known ligands. Other reporters than luciferase can be used including CAT and other enzymes. In one specific embodiments of this approach, the cells further express PGC-1α or PGC-1β, either endogenously or by introduction of a third plasmid encoding said polypeptides. The presence of PGC-1 polypeptides in the cell further allows for the identification of agents which increase or decrease the binding interaction between a PGC-1 polypeptide and Errα. This approach may also be modified to express both Gabpa and Gabpb to identify agents which modulate their transcriptional activity. Alternatively, a cell may be used which endogenously expresses any combination of polypeptides, such that only a plasmid encoding a reporter gene is introduced into the cell.

Viral constructs can be used to introduce the gene for Errα Gabp or PGC-1 and the reporter into a cell. An usual viral vector is an adenovirus. For further details concerning this preferred assay, see U.S. Pat. No. 4,981,784 issued Jan. 1, 1991 hereby incorporated by reference, and Evans et al., WO88/03168 published on 5 May 1988, also incorporated by reference.

Errα antagonists can be identified using this same basic “agonist” assay. A fixed amount of an antagonist is added to the cells with varying amounts of test compound to generate a dose response curve. If the compound is an antagonist, expression of luciferase is suppressed.

Additional methods for the isolation of agonists and antagonist of transcriptional regulators are described in U.S. Pat. Nos. 6,187,533, 5,620,887, 5,804,374, and 5,298,429, and U.S. Patent Publication Nos. 2004/003394, 2003/0077664, 2003/0215829 and 2003/0039980. Any of the methods described herein may be easily adapted to identify agonists or antagonists of any one Errα or Gabp polypeptides.

U.S. Pat. No. 6,555,326 (PCT Pub No. WO 99/27365) describes a fluorescent polarization assay for identifying agents which regulate the activity of nuclear hormone receptors, by using a nuclear hormone receptor, a peptide sensor and a candidate agent. Table 1 of this patent also lists exemplary nuclear hormone receptors. Such a method may easily be modified by one skilled in the art to identify agents which regulate the activity of Errα or Gabp.

The invention also provides a method for screening a candidate compound for its ability to modulate Errα activity in a suitable system, in the presence or absence of the candidate compound. A change in Errα activity the presence of the compound relative to ERRα activity in the absence of the compound indicates that the compound modulates ERRα activity. ERRα activity is increased relative to the control in the presence of the compound, the compound is an ERRS agonist. Conversely, if ERRS activity is decreased in the presence of the compound, the compound is an ERRα antagonist.

Another way of determining if an agent increases the activity of Errα or Gabp may also be based on binding of the agent to an ERRα or to a Gabp polypeptide or fragment thereof. Such competitive binding assays are well known to those skilled in the art.

For example, the invention provides screening methods for compounds able to bind to ERRα which are therefore candidates for modifying the activity of ERRα. Various suitable screening methods are known to those in the art, including immobilization of ERRα on a substrate and exposure of the bound ERRα to candidate compounds, followed by elution of compounds which have bound to the ERRα. Additional methods and assays for identifying agents which modulate Errα activity, for generating Errα knock out animals and cells, and for generating Errα reagents, such as anti-Errα antibodies are described in International PCT publication No. WO 00/122988, hereby incorporated by reference in its entirety.

Another aspect of the invention provides a method of identifying an agent that modulates a biological response, the method comprising (a) contacting, in the presence of the agent, a PGC-1 polypeptide and an (i) Errα polypeptide, or (ii) a Gabp polypeptide, under conditions which allow the formation of a complex between the PGC-1 polypeptide and (i) the Errα polypeptide, or (ii) the Gabp polypeptide; and (b) detecting the presence of the complex; wherein an agent that modulates the biological response is identified if the agent increases or decreases the formation of the complex, and wherein the biological response is (a) expression of the OXPHOS genes; (b) mitochondrial biogenesis; (c) expression of Nuclear Respiratory Factor 1 (NRF-1); (d) β-oxidation of fatty acids; (e) total mitochondrial respiration; (f) uncoupled respiration; (g) mitochondrial DNA replication; or (h) expression of mitochondrial enzymes.

In some embodiments of the methods for identifying an agent that modulates a biological response, the method comprises an agent that increases the formation of the complex and that increases the biological response. In alternate embodiments, the agent decreases the formation of the complex and decreases the biological response. In some embodiments, the conditions which allow the formation of a complex between the PGC-1 polypeptide and an Errα polypeptide or a Gabpa polypeptide comprise in vitro conditions, while in other embodiments they comprise in vivo conditions such as expression in a cell or in an organism.

The following embodiments of methods for identifying a compound that modulates a biological response, although directed at Errα and PGC-1α, are equally applicable to Gabp polypeptides, such as Gabpa polypeptides, or to PGC-1β polypeptides.

One embodiment for the of the methods for identifying a compound that modulates a biological response comprises: 1) combining: a Errα polypeptide or fragment thereof, a PGC-1α polypeptide or fragment thereof, and an agent, under conditions wherein the Err alpha and PGC-1α polypeptides physically interact in the absence of the agent, 2) determining if the agent interferes with the interaction, and 3) for an agent that interferes with the interaction, further assessing its ability to promote the any of the biological responses of the cell, such as (a) expression of the OXPHOS genes, mitochondrial biogenesis, expression of Nuclear Respiratory Factor 1 (NRF-1), β-oxidation of fatty acids, total mitochondrial respiration, uncoupled respiration, mitochondrial DNA replication or expression of mitochondrial enzymes.

A variety of assay formats will suffice and, in light of the present disclosure; those not expressly described herein will nevertheless be comprehended by one of ordinary skill in the art. Assay formats which approximate such conditions as formation of protein complexes, enzymatic activity, may be generated in many different forms, and include assays based on cell-free systems, e.g. purified proteins or cell lysates, as well as cell-based assays which utilize intact cells. Simple binding assays can also be used to detect agents which bind to Errα or PGC-1α. Such binding assays may also identify agents that act by disrupting the interaction between a Errα polypeptide and PGC-1α. Agents to be tested can be produced, for example, by bacteria, yeast or other organisms (e.g. natural products), produced chemically (e.g. small molecules, including peptidomimetics), or produced recombinantly. Because Errα and PGC-1a polypeptides contain multiple domains, specific embodiments of the assays and methods described to identify agents which modulate complex formation between Errα and PGC-1a employ fragments of Errα rather than full-length polypeptides, such as those lacking the DNA binding domains. Fragments of PGC-1α may also be used in some embodiments, in particular fragments which retain the ability to complex with Errα.

In many drug screening programs which test libraries of compounds and natural extracts, high throughput assays are desirable in order to maximize the number of compounds surveyed in a given period of time. Assays of the present invention which are performed in cell-free systems, which may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test agent can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted Errα/PGC-1α complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in Errα/PGC-1α complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure Errα/PGC-1α complex assembly and/or disassembly.

Assaying Errα/PGC-1α complexes, in the presence and absence of a candidate agent, can be accomplished in any vessel suitable for containing the reactants. Examples include microtiter plates, test tubes, and micro-centrifuge tubes. In a screening assay, the effect of a test agent may be assessed by, for example, determining the effect of the test agent on kinetics, steady-state and/or endpoint of the reaction.

In one embodiment of the present invention, drug screening assays can be generated which detect inhibitory agents on the basis of their ability to interfere with assembly or stability of the Errα/PGC-1a complex. In an exemplary binding assay, the compound of interest is contacted with a mixture comprising a Errα/PGC-1a complex. Detection and quantification of Errα/PGC-1α complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation may be detected by a variety of techniques. For instance, modulation in the formation of complexes can be quantitated using, for example, detectably labeled proteins (e.g. radiolabeled, fluorescently labeled, or enzymatically labeled), by immunoassay, or by chromatographic detection. Surface plasmon resonance systems, such as those available from Biacore © International AB (Uppsala, Sweden), may also be used to detect protein-protein interaction.

The proteins and peptides described herein may be immobilized. Often, it will be desirable to immobilize the peptides and polypeptides to facilitate separation of complexes from uncomplexed forms of one of the proteins, as well as to accommodate automation of the assay. The peptides and polypeptides can be immobilized on any solid matrix, such as a plate, a bead or a filter. The peptide or polypeptide can be immobilized on a matrix which contains reactive groups that bind to the polypeptide. Alternatively or in combination, reactive groups such as cysteines in the protein can react and bind to the matrix. In another embodiment, the polypeptide may be expressed as a fusion protein with another polypeptide which has a high binding affinity to the matrix, such as a fusion protein to streptavidin which binds biotin with high affinity.

In an illustrative embodiment, a fusion protein can be provided which adds a domain that permits the protein to be bound to an insoluble matrix. For example, a GST-ERRα fusion protein can be adsorbed onto glutathione sepharose beads (Sigma Chemical, St. Louis, Mo.) or glutathione derivatized microtitre plates, which are then combined with a PGC-1a polypeptide, e.g. an ³⁵S-labeled polypeptide, and the test compound and incubated under conditions conducive to complex formation. Following incubation, the beads are washed to remove any unbound interacting protein, and the matrix bead-bound radiolabel determined directly (e.g. beads placed in scintillant), or in the supernatant after the complexes are dissociated, e.g. when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In yet another embodiment, the Errα and PGC-1α polypeptides can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14: 920-924; and Iwabuchi et al. (1993) Oncogene 8:1693-1696), for subsequently detecting agents which disrupt binding of the proteins to one and other.

In still further embodiments of the present assay, the Errα/PGC-1α complex is generated in whole cells, taking advantage of cell culture techniques to support the subject assay. For example, as described below, the Errα/PGC-1α complex can be constituted in a eukaryotic cell culture system, such as a mammalian cell and a yeast cell. Other cells known to one skilled in the art may be used. Advantages to generating the subject assay in a whole cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the Errα/PGC-1a complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In still further embodiments of the present assay, the Errα/PGC-1a complex is generated in whole cells and the level of interaction is determined by measuring the level of gene expression of an (i) endogenous gene or of a transgene, whose expression is dependent on the formation of a complex. Genes which are responsive to Errα/PGC-1a complex are provided by the invention and some may be found in the literature.

In specific embodiments, the cells used in the methods described herein for identifying agents are cells in culture or from a subject, such as a tissue, fluid or organ or a portion of any of the foregoing. For example, cells can preferably be from tissues that are involved in glucose metabolism, such as pancreatic cells, islates of Langerhans, pancreatic beta cells, muscle cells, liver cells or other appropriate cells. Preferably, cells are provided in culture and can be a primary cell line or a continuous cell line and can be provided as a clonal population of cells or a mixed population of cells.

VII. Methods of Identifying Agents which Modulate OXPHOS-CR Expression

Applicants have identified a core set of genes (OXPHOS-CR) that help unify previous observations from clinical investigation, exercise physiology, pharmacology, and genetics. Drugs that modulate OXPHOS-CR activity may be promising candidates for the prevention and/or treatment of type 2 diabetes. Applicants discovery of OXPHOS-CR properties and previous observations support the hypothesis that drugs that increase OXPHOS-CR activity in muscle and fat will improve insulin resistance, while agents that reduce it will worsen insulin resistance. These drugs may have benefit in other processes characterized by aberrant oxidative capacity in these tissues, including obesity and aging.

The methods described in this section for identifying agents which regulate the expression level of one or more OXPHOS-CR genes may also identify agents which modulate PGC-1α, Gabp or Errα expression or activity, or agents which mimic or functionally substitute for these genes, since applicants have demonstrated that these three transcriptional regulators regulate the expression of OXPHOS-CR genes. Likewise, these methods also identify therapeutic agents which modulate metabolism or mitochondrial function in a subject in need thereof, such as a subject afflicted with diabetes.

Accordingly, the invention further provides cell based methods for identifying agents which regulate the expression of OXPHOS-CR genes, On aspect provides a method of identifying an agent that regulates expression of OXPHOS-CR genes, the method comprising (a) contacting (i) an agent to be assessed for its ability to regulate expression of OXPHOS-CR genes with (ii) a test cell; and (b) determining whether the expression level of at least two OXPHOS-CR gene products show a coordinate change in the test cell compared to an appropriate control, wherein a coordinate change in the expression of the OXPHOS-CR gene products relative to the appropriate control indicates that the agent regulates the expression of OXPHOS-CR genes.

A related aspect of the invention provides method of identifying an agent that regulates expression of a gene, wherein the gene is an OXPHOS-CR gene, the method comprising (a) contacting (i) an agent to be assessed for its ability to regulate expression of the gene with (ii) a test cell; and (b) determining whether the expression level of two or more OXPHOS-CR gene products show a coordinate change in the test cell compared to an appropriate control, wherein the gene does not encode the two or more OXPHOS-CR gene products, and wherein a coordinate change in the expression of the OXPHOS-CR gene products relative to the appropriate control indicates that the agent regulates the expression level of the gene.

In some embodiments, the OXPHOS-CR gene products comprise an mRNA or a polypeptide. The gene products of the two genes need not be of the same type. For instance, in one specific embodiment, the mRNA levels of a first OXPHOS-CR gene, the polypeptide levels of a second OPHOS-CR gene, and the enzymatic activity of a third OXPHOS-CR genes are determined. In a preferred embodiment, all the gene products comprises mRNAs.

In additional embodiments, determining whether the expression of at least two OXPHOS-CR gene products show a coordinate change in the test cell comprises detecting, either qualitatively, semiquantitatively, or more preferably quantitatively, the levels of the OXPHOS-CR gene products. In one embodiment, the coordinate change comprises an increase or a decrease in expression in all the genes tested. In another embodiment, a coordinate change comprises an increase or a decrease in at least 60%, 65%, 70%, 75%, 80%, 85%, 90%, 91%, 92%, 93%, 94%, 95%, 95%, 97%, 98% or 99% of the genes tested.

In a variation of this method, more than one cell is contacted with the agent. In yet another variation, multiple cells or cell populations are contacted with the agent, such that each cell or cell population provides a measure of expression for each of the OXPHOS-CR gene products. For example, if the expression level of four OXPHOS-CR genes is to be determined, then four cell populations, such as one on each well of a 96-well plate, is contacted with the agent, and from each well the expression level of one of the OXPHOS genes is determined. Alternatively, two cell populations could be used and the expression level of two gene products could be determined from each of the two cell populations. In another embodiment, the cell or cell population is contacted with more than one agent.

The expression level of the OXPHOS-CR gene products may be determined using techniques known in the art. Gene products which comprise an mRNA may be detected, for example, using reverse transcriptase mediated polymerase chain reaction (RT-PCR), Northern blot analysis, in situ hybridization, microarray analysis, etc. (Schena et al., Science 270:467-470 (1995); Lockhart et al., Nature Biotech. 14: 1675-1680 (1996), and U.S. Pat. Nos. 5,770,151, 5,807,522, 5,837,832, 5,952,180, 6,040,138 and 6,045,996). Polypeptide products may be detected using, for example, standard immunoassay methods known in the art. Such immunoassays include but are not limited to, competitive and non-competitive assay systems using techniques such as radioimmunoassays, ELISA (enzyme-linked immunosorbent assay), “sandwich” immunoassays, immunoradiometric assays, gel diffusion precipitin, reactions, immunodiffusion assays, in situ immunoassays (using colloidal gold, enzymatic, or radioisotope labels, for example), Western blots, 2-dimensional gel analysis, precipitation reactions, immunofluorescence assays, protein A assays, and immunoelectrophoresis assays.

When the gene product comprises an enzyme, the level of gene product may be determined using a measure of enzymatic activity. Products of enzyme catalytic activity may be detected by suitable methods that will depend on the quantity and physicochemical properties of the particular product. Thus, detection may be, for example by way of illustration and not limitation, by radiometric, calorimetric, spectrophotometric, fluorimetric, immunometric or mass spectrometric procedures, or by other suitable means that will be readily apparent to a person having ordinary skill in the art. In certain embodiments of the invention, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

In one embodiment, the promoter regions for two or more OXPHOS-CR genes (or larger portions of such genes) may be operatively linked to a reporter gene and used in a reporter gene-based assay to detect agents that enhance or diminish OXPHOS-CR gene expression. In such embodiments, the OXPHOS gene product is the mRNA or polypeptide encoded by the reporter gene. In a specific embodiment, the recombinant fluorescent polypeptide comprises a polypeptide selected from the group consisting of the green fluorescent protein (GFP), DsRed, zFP538, mRRFP1, BFP, CFP, YFP, mutants thereof, or functionally-active fragments thereof. GFP is described in U.S. Pat. No. 5,491,084, while zFP538 is described in Zagranichny et al. Biochemistry. 2004; 43(16):4764-72.

In another specific embodiment, the appropriate control comprises the expression level of the two or more OXPHOS-CR gene products in cells that (a) have not been contacted with the agent; (b) have been contacted with a different dosage of the agent; (c) have been contacted with a second agent; or (d) a combination thereof. Alternatively, an appropriate control may be a measure of the gene product in the cell prior to contacting with the agent. In another embodiment, the level of gene expression of the OXPHOS-CR gene product in the cell can be compared with a standard (e.g., presence or absence of an OXPHOS-CR gene product) or numerical value determined (e.g. from analysis of other samples) to correlate with a normal or expected level of expression.

In some embodiments, the identification of agents which regulate the expression of OXPHOS-CR genes is carried out in a high-throughput fashion. When screening agents in a high-throughput manner, such as when test compounds are screened for their effects on the cellular phenotype, arrays of cells may be prepared for parallel handling of cells and reagents. Standard 96 well microtiter plates which are 86 mm by 129 mm, with 6 mm diameter wells on a 9 mm pitch, may be used for compatibility with current automated loading and robotic handling systems, The microplate is typically 20 mm by 30 mm, with cell locations that are 100-200 microns in dimension on a pitch of about 500 microns. Methods for making microplates are described in U.S. Pat. No. 6,103,479, incorporated by reference herein in its entirety. Microplates may consist of coplanar layers of materials to which cells adhere, patterned with materials to which cells will not adhere, or etched 3-dimensional surfaces of similarly pattered materials. For the purpose of the following discussion, the terms ‘well’ and ‘microwell’ refer to a location in an array of any construction to which cells adhere and within which the cells are imaged. Microplates may also include fluid delivery channels in the spaces between the wells. The smaller format of a microplate increases the overall efficiency of the system by minimizing the quantities of the reagents, storage and handling during preparation and the overall movement required for the scanning operation. In addition, the whole area of the microplate can be imaged more efficiently.

In specific embodiments, the test cell that is contacted with the agent may be a primary cell, a cell within a tissue, or a cell line. In a preferred embodiment, the test cell is a liver cell, a skeletal muscle cell, such as a C2C12 myoblast or a fat cell, such as 3T3-L1 preadipocyte.

In one embodiment, the method for identifying an agent that regulates expression of OXPHOS-CR genes comprises determining whether the expression of at least 3, 4, 5, 6, 7, 8, 9, 10, 11, 12, 13, 14, 15, 16, 17, 18, 19, 20, 21, 22, 23, 24, 25, 26 or 27 OXPHOS-CR gene products. In a preferred embodiment, the expression level of five or less OXPHOS-CR gene products is determined. In a specific embodiment, the OXPHOS-CR gene products are selected from the group consisting of NDUFB3, SDHA, NDUFA8, COX7A1, UQCRC1, NDUFC1, NDUFS2, ATP50, NDUFS3, SDHB, NDUFS5, NDUFB6, COX5B, CYC1, NDUFA7, UQCRB, COX7B, ATP5L, COX7C, NDUFA5, GRIM19, ATP5J, COX6A2 NDUFB5, CYCS, NDUFA2 and HSPC051. In a specific embodiment, one of the OXPHOS-CR genes is ubiquinol cytochrome c reductase binding protein (UQCRB). In a preferred embodiment, the OXPHOS-CR gene products are human OXPHOS-CR products. The OXPHOS-CR genes whose expression level is determined may be encoded by (i) mitochondrial DNA (mtDNA); (ii) nuclear DNA; or (iii) a combination thereof.

In one embodiment of the methods described herein for identifying agents which regulate the expression of OXPHOS-CR genes, the method further comprises determining if the agent regulates the expression of at least one gene which is not an OXPHOS-CR gene. In some embodiments, the method further comprises determining if the agent regulates the expression of at least 2, 3, 4, 5, 6, 7, 8, 9, 10, 15, 20, 25 or 50 genes which are not an OXPHOS-CR genes. Such genes may be mitochondrial genes or, in preferred embodiments, not mitochondrial genes, such as actin genes. The expression level of another gene which is not an OXPHOS-CR gene may serve as an internal control, such that agents which specifically modulate the expression of an OXPHOS-CR gene may be identified.

In other embodiments, a secondary screening step is performed on the agent. In a specific embodiment, the agent is tested in additional assays for its effects on mitochondrial cell number or a mitochondrial function, such as coupled oxygen consumption. Such assays may comprise contacting a cell with the agent, measuring mitochondrial cell number or function, and comparing it to an appropriate control. U.S. Patent Publication No. 20020049176 describes assays for determining mitochondrial mass, volume or number, and U.S. Patent Publication No. 2002/0127536 describes assays for determining coupled oxygen consumption. Accordingly, in one embodiment, the agent being tested in the assays described herein additionally (a) increases the number of mitochondria in the test cell; (b) increases coupled oxygen consumption in the cell; (c) increases mtDNA copy number in the test cell; or (d) a combination thereof.

Agents identified using the methods of the present invention may also be tested in model systems for their efficacy in inducing the desired biological response or in treating disorders. One example is high-fat diet induced obesity and insulin resistance. In another example, agents may also be tested for their efficacy in treating diabetes by using a non-obese diabetic (NOD) mouse. The successful use of this animal model in diabetic drug discovery is reported in the literature (Yang et al., J. Autoimmun. 10:257-260 (1997), Akashi et al., Int. Immunol. 9:1159-1164 (1997), Suri and Katz, Immunol. Rev. 169:55-65 (1999), Pak et al., Autoimmunity 20:19-24 (1995), Toyoda and Formby, Bioessays 20:750-757 (1998), Cohen, Res. Immunol. 148:286-291 (1997), Baxter and Cooke, Diabetes Metal. Rev. 11:315-335 (1995), McDuffie, Curr. Opin. Immunol. 10:704-709 (1998), Shieh et al. Autoimmunity 15:123-135 (1993), Anderson et al., Autoimmunity 15:113-122 (1993)).

It is well understood by one skilled in the art that many of the methods described herein may be carried out using variants of the polypeptides described. Variants include truncated polypeptides, mutant polypeptides, such as those carrying point mutations, and fusions between domains of the subject polypeptides and other polypeptides. In some embodiments, the subject polypeptides, or their domains, may be fused to reporter proteins, such as to GFP or to enzymes. In some embodiments of any of the methods described herein, the polypeptides used are 50, 60, 70, 80, 90, 95, 98 or 99% identical to the sequences referenced to in the various Genbank Accession numbers.

In the methods described herein for identifying an agent, the agent may comprise a recombinant polypeptide, a synthetic molecule, or a purified or partially purified naturally occurring molecule. In a specific embodiment, the agent comprises a virus or a phage. In another embodiment, the agent is a nuclear hormone, such as estrogen, thyroid hormone, cortisol, testosterone, and others. Additional agents include nucleic acids encoding nuclear hormone receptors.

In another embodiment, the agent comprises a set of environmental conditions. The condition may be a physical condition of the environment in which the cell resides, a chemical condition of the environment, and/or a biological condition of the site. Exposure may be for any suitable time. The exposure may be continuous, transient, periodic, sporadic, etc. Physical conditions include any physical state of the examination site. The physical state may be the temperature or pressure of the sample, or an amount or quality of light (electromagnetic radiation) at the site. Alternatively, or in addition, the physical state may relate to an electric field, magnetic field, and/or particle radiation at the site, among others. Chemical conditions include any chemical aspect of the fluid in which the sample populations are disposed. The chemical aspect may relate to presence or concentration of a test compound or material, pH, ionic strength, and/or fluid composition, among others.

Biological conditions include any biological aspect of the shared fluid volume in which cell populations are disposed. The biological aspects may include the presence, absence, concentration, activity, or type of cells, viruses, vesicles, organelles, biological extracts, and/or biological mixtures, among others. The assays described herein may screen a library of conditions to test the activity of each library member on a set of cell populations. A library generally comprises a collection of two or more different members. These members may be chemical modulators (or candidate modulators) in the form of molecules, ligands, compounds, transfection materials, receptors, antibodies, and/or cells (phages, viruses, whole cells, tissues, and/or cell extracts), among others, related by any suitable or desired common characteristic. This common characteristic may be “type.” Thus, the library may comprise a collection of two or more compounds, two or more different cells, two or more different antibodies, two or more different nucleic acids, two or more different ligands, two or more different receptors, or two or more different phages or whole cell populations distinguished by expressing different proteins, among others. This common characteristic also may be “function.” Thus, the library may comprise a collection of two or more binding partners (e.g., ligands and/or receptors), agonists, or antagonists, among others, independent of type.

Library members may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (about 100 Da to 10 KDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others. Library members may be contact arrays of cell populations singly or as groups/pools of two or more members.

VIII. Methods of Identifying Transcriptional Regulators

Another aspect of the invention provides methods of identifying transcriptional regulators. In some aspects, the invention provides methods of identifying transcriptional regulators which display differential activity between two cells.

The invention provides a method of identifying a transcriptional regulator having differential activity between an experimental cell and a control cell, the method comprising (i) determining the level of gene expression of at least two genes in the experimental cell and in the control cell; (ii) ranking genes according to a difference metric of their expression level in the experimental cell compared to the control cell; (iii) identifying a subset of genes, wherein each gene in the subset contains the same DNA sequence motif; (iv) testing via a nonparametric statistic if the subset of genes are enriched at either the top or the bottom of the ranking; (v) optionally reiterating steps (ii)-(iii) for additional motifs; (vi) for a subset of genes that is enriched, identifying a transcriptional regulator which binds to a DNA sequence motif that is contained in the subset of genes; thereby identifying a transcriptional regulator having differential activity between two cells.

The methods provided by the invention for identifying transcriptional regulators with differential activity are not limited to any type of cell or to any type of difference between the two cell. The cells may be eukaryotic, prokaryotic, yeast, nematode, insect, mammalian or human cells. The cells may be primary cells, or cell lines. The cells may be in an organism. In one specific embodiment, the cells are isolated from a subject.

The control and the experimental cell may be the same type of cell or they may be different types of cells. In one embodiment, the experimental cell and the control cell are both cells derived from the same cell line or from the same tissue types. In some embodiments, the experimental cell and the control cell are from different organisms, such as from two different subjects. In some specific embodiments in which the cells are derived from the same organism, one cell is a normal cell and another cell is a diseased cell. For instance, one cell may be a cancer cell and one may be a non-cancer cell, or one cell may be a virus infected cell and one may be a non-infected cell. In some embodiments, both cells may be diseased cells, but differ in their disease states. For instance, the two cells may be hyperplastic cells but at different stages of cancer progression e.g. one cell may be a tumor cell and the other a metastatic cell derived from that tumor. Furthermore, the two cells may differ genetically or they may be clonal cells with essentially identical genotypes. One or both of the cells may be experimentally manipulated, such as by contacting one of the cells with an agent, or contacting both cells with an agent but at different concentrations.

In some embodiments of the method, the subject from which one or both of the cells are derived in is afflicted with a disorder. The method is not limited by any particular disorder. In some specific embodiments, the disorder is a metabolic disorder or a hyperplastic condition. Hyperplastic conditions include renal cell cancer, Kaposi's sarcoma, chronic leukemia, prostate cancer, breast cancer, sarcoma, pancreatic cancer, leukemia, ovarian carcinoma, rectal cancer, throat cancer, melanoma, colon cancer, bladder cancer, lymphoma, mastocytoma, lung cancer, mammary adenocarcinoma, pharyngeal squamous cell carcinoma, testicular cancer, gastrointestinal cancer, or stomach cancer, or a combination thereof. Additional disorders to which this method may be applied may be found, for example, in Braunwald, E. et al. eds. Harrison's Principles of Internal Medicine, 15^{th} Edition (McGraw-Hill Book Company, New York, 2001).

In some embodiments, a transgene is introduced into the experimental cell. The transgene may encode any protein, such as transcriptional regulators or proteins that regulate the activity of transcriptional regulators, such as kinase and phosphatases. The transgene may also encode an inhibitory RNA, such as a hairpin RNA, so that the function of the gene to which the hairpin RNA is directed may be knocked down, allowing a comparison of gene expression in between the two cells. In some embodiments, the transgenes is a transgene associated with a disease state. For example, a gene whose overexpressing leads to cancer may be overexpressed to identify transcriptional regulators expressing differential activity between the two cells. These transcriptional regulators may then be used as therapeutic targets for the treatment of cancer. In some embodiments, the transgene is a mutant transgene, such as a mutant transgene associated with a disease state.

In some embodiments, the DNA sequence motif comprises at least 5, 6, 7, 8, 9, 10, 11, 12, 13, 14, 15, 16, 17, 18, 19, 20, or 25 nucleotides in length, preferably at least 5. The DNA sequence motif may be any combination of nucleotides, and it may represent a known binding site or a novel binding site. In some embodiments, the DNA sequence motif comprises undefined nucleotide positions which may contain more than one base. For instance, a DNA sequence motif may comprise the sequence GATNNATC, wherein the 3^{rd} and 4^{th} positions would include any of the four bases. Similarly, a DNA sequence motif comprising the sequence GAT(G/T)ATC would have a G or a T in the fourth position. In some embodiments, DNA sequence motif comprises at least 5, 6, 7, 8, 9, 10, 11, 12, 13, 14, 15, 16, 17, 18, 19, or 20 defined positions.

The method can be applied to any number of motifs. In one embodiment, all permutations of DNA sequence motifs of at least 6, 7, 8 and 9 bases in length are tested. The number selected may depend on the number of genes in the subset, the computational capabilities available, and the size of the window in each gene in which the DNA sequence motif is search.

The method is not limited to any particular method of measuring gene expression. In some embodiments, determining the level of expression of a gene in a cell comprises determining the levels of mRNA for the gene in the cell. Any method known in the art may be used to determine mRNA levels. In one embodiment, mRNA is isolated from the cell, and the levels of mRNA for each gene in the subset is determined by hybridizing the mRNA, or cDNA derived from the mRNA, to a DNA microarray.

In some embodiments of the methods described herein, identifying the transcriptional regulator which binds to a DNA sequence motif comprises searching a database comprising transcriptional regulators and DNA sequence motifs to which they bind. For example, the TRANSFAC transcription factor database, maintained at the GBF Braunschweig, Germany, defines sequence specific binding site patterns, or motifs, for transcription factors. In another embodiment, the transcriptional regulator is identified by comparing the sequences identified to those found in the literature. It is understood by one skilled in the art that more than one transcriptional regulator may bind to a given DNA sequence motif, and therefore multiple transcriptional regulators may be identified.

In some embodiments of the method described herein, identifying a transcriptional regulator which binds to a DNA sequence motif comprises experimentally identifying a transcriptional regulator which binds to the DNA sequence motif. In one embodiment, this is achieved by These may be achieved by (i) identifying, from a library of genes, a transcriptional regulator capable of driving the expression of a selectable marker, wherein the expression of the selectable marker is dependent on binding of the transcriptional regulator to the DNA sequence motif. In a specific embodiment, a reporter gene is introduced into a cell, such as a mammalian cell or a yeast cell, wherein the promoter of the reporter gene is operably linked to the DNA sequence motif. A plasmid library which comprises candidate transcriptional regulator genes is introduced into the cells such that the transcriptional regulators are expressed in the cell. If a transcriptional regulator is able to bind to the DNA sequence motif, it will increase or decrease expression of the reporter gene, allowing identification of the cell expressing said regulator and thus allowing its identification. In a specific embodiment, a yeast one-hybrid approach, or other approaches well known to one skilled in the art, is used to identify a transcriptional regulator which binds to the DNA sequence motif (Vidal M et al. Nucleic Acids Res. 1999; 27(4):919-29, Kadonaga et al., (1986) Proc. Natl. Acad. Sci. USA, 83, 5889-5893. Singh et el. (1988) Cell, 52, 415-423; Chong, J. A. et al. (1997) In Bartel, P. L. and Fields, S. (eds), The Yeast Two-Hybrid System. Oxford University Press, New York, N.Y., pp. 289-297). Transcriptional regulators may also be identified based on its binding affinity for the DNA sequence motif, such by standard affinity chromatography.

In some embodiments, the non-parametric statistic is a nonparametric, rank sum statistic. In specific embodiments, the non-parametric statistic is selected from the group consisting of a Kolmogorov-Smirnov, Mann-Whitney or Wald-Wolfowitz. Non-parametric statistics are well-known in the art (David J. Sheskin, Handbook of Parametric and Nonparametric Statistical Procedures, CRC Press, 2003; Myles Hollander, Douglas A. Wolfe, Nonparametric Statistical Methods, Wiley, John & Sons, Inc., 1998; Larry Wasserman, All of Statistics, Springer-Verlag New York, Incorporated, 2003). In some embodiments, the difference metric is a difference in arithmetic means, t-test scores, or signal to noise ratios. In some embodiments, a gene set is said to be enriched if the probability that the gene set would be enriched by chance, or when compared to an appropriate null hypothesis, is less than 0.05, 0.04, 0.03, 0.02, 0.01, 0.005, 0.0001, 0.00005 or 0.00001.

In some embodiments where the experimental cell expresses a recombinant transgene, such as a recombinant transcriptional regulator, the recombinant transcriptional regulator may itself be found to have differential activity. In other embodiments where the experimental cell expresses a recombinant transgene, the method may yield transcriptional regulators whose activity or expression is itself regulated by the recombinant transcriptional regulator, and if a recombinant transcriptional regulator is used whose activity is related to a disease state is used, identification of transcriptional regulators having differential activity between the two cells may yield therapeutic targets to treat the disorder.

IX. Biomarker Set Enrichment Analysis (BSEA)

One aspect of the invention provides methods of detecting statistically-significant differences in the expression level of at least one biomarker belonging to a biomarker set, between the members of a first and of a second experimental group. Applicants have named this new analytical technique Biomarker Set Enrichment Analysis (BSEA), or Gene Set Enrichment Analysis (GSEA) when the biomarker is a gene or a gene product.

GSEA may be valuable in efforts to relate genomic variation to disease and measures of total body physiology. Single-gene methods are powerful only where the individual gene effect is dramatic and the variance small, which may not be the case in many disease states. Methods like GSEA are complementary, and provide a framework with which to examine changes operating at a higher level of biological organization. This may be needed if common, complex disorders typically result from modest variation in the expression or activity of multiple members of a pathway e.g. gene (biomarker) sets. As gene sets are systematically assembled using functional and genomic approaches, methods such as GSEA will likely be valuable in detecting coordinated but subtle variation in gene function that contribute to common human diseases. Accordingly, in a preferred embodiment, the methods detect statistically-significant differences in the expression level in more than one biomarker.

One aspect of the invention provides a method of detecting statistically-significant differences in the expression level of at least one biomarker belonging to a biomarker set, between the members of a first and of a second experimental group, comprising: (a) obtaining a biomarker sample from members of the first and the second experimental groups; (b) determining, for each biomarker sample, the expression levels of at least one biomarker belonging to the biomarker set and of at least one biomarker not belonging to the set; (c) generating a rank order of each biomarker according to a difference metric of its expression level in the first experimental group compared to the second experimental group; (d) calculating an experimental enrichment score for the biomarker set by applying a non parametric statistic; and (e) comparing the experimental enrichment score with a distribution of randomized enrichment scores to calculate the fraction of randomized enrichment scores greater than the experimental enrichment score, wherein a low fraction indicates a statistically-significant difference in the expression level of the biomarker set between the members of the first and of the second experimental group.

In one embodiment of the foregoing methods, the distribution of randomized enrichment scores is generated by randomly permutating the assignment of each biomarker sample to the first or to the second experimental group; (ii) generating a rank order of each biomarker according to the absolute value of a difference metric of its expression level in the first experimental group compared to the second experimental group; (iii) calculating an experimental enrichment score for the biomarker set by applying a non parametric statistic to the rank order; and (iv) repeating steps (i), (ii) and (iii) a number of times sufficient to generate the distribution of randomized enrichment scores. In a specific embodiment, the number of times sufficient to generate a distribution is at least 20, 30, 40, 50, 60, 70, 80, 90, 100, 150, 200 or 500 times. In another specific embodiment, the low fraction is less than 0.05, while in other embodiments it is less than 0.04, 0.03, 0.02, 0.01, 0.005 or 0.001.

In one embodiment of the foregoing methods, the distribution of randomized enrichment scores is a normal distribution. The difference metric may be any difference metric, such as a difference in arithmetic means, a difference in t-test scores, or a difference in signal-to-noise ratio. Similarly, the non-parametric statistic may be any non-parametric statistic, such Mann-Whitney, Wald-Wolfowitz or more preferably Kolmogorov-Smirnov.

The biomarker set typically comprises elements of a pathway, such as a metabolic pathway, a biochemical pathway, a signaling pathway, or any set of genes which share a common biological function or which are coordinately regulated. In a preferred embodiment, the biomarker is selected from the group consisting of a nucleic acid, a polypeptide, a metabolite and a genotype. For example, when the biomarker set comprises genes encoding enzymes of a metabolic pathway, such as glycolytic enzymes, the biomarkers may comprise the genotype of the glycolytic genes. In the embodiment where the biomarker is a genotype, the genotype of all or a subset of the glycolytic genes may be determined by DNA sequencing, and the expression level of the genotype would correspond to the amount of polymorphic DNA i.e. 0, 1 or 2 copies of a wild-type copy of the gene for a diploid cell or organism. Alternatively, the number of mutant copies, or of a specific mutation, can be used in determining the expression level of the genotype.

In other embodiments where the biomarker is the mRNA of each of, or of a subset of, the glycolytic enzymes, the expression level of the mRNA may be determined, or the expression level of a particular splice isoform, using methods well known in the art, such as by northern blots or microarray analysis. In other embodiments where the biomarker is the protein of each of, or of a subset of, the glycolytic enzymes, the level of expression may comprise total protein levels or levels of a particular modified form of the protein, such as the level of phosphorylated or glycosylated protein, both of which may be determined using immunological techniques. Finally, when the biomarker is a metabolite, such as the product whose formation is catalyzed by the glycolytic enzyme, the expression level of the metabolite is its concentration in the biomarker sample, such as its cellular concentration. Metabolite levels may be determined using chromatographic means or other means well known in the art. The reference to the glycolitic pathway in the examples above is meant to be illustrative and non-limiting, or the same principles may apply to any other pathway or biomarker set.

In one embodiment, experimental groups comprise organisms, such as mammals, or more preferably humans. In such embodiments, the sample from the biomarker sample comprises a sample of cells from the organism, or a sample of bodily fluid, such as serum, saliva, tears, sweat or semen. The difference between the first and second experimental groups may be a disease state. For example, the first experimental group may be afflicted with a disease or disorder, while the second group is not. In a specific embodiment, the disorder is characterized by defective glucose metabolism, such as type II diabetes. In another embodiment where the experimental groups comprise organisms, the first and second experimental groups may differ by any measurable characteristic. For example, the groups may differ by a physical characteristic, such as weight, age, sex, sexual preference, eyesight, percent body fat, percent lean muscle mass, height, right vs. left handedness or race. The groups may also differ by a psychological characteristic, such as intelligence, verbal skills, emotional intelligence and even personality types, such those determined by the Myers-Briggs Type Indicator. The groups may also differ by emotional state, such as relaxed vs. emotionally stressed subjects, or cheerful vs. gloomy subjects. The subjects may also differ by the presence or absence of one or more mutations, such as subjects having mutations in an oncogene. In another embodiment, the two experimental groups differ in that one group has been treated with at least one agent, such as a drug.

In another embodiment, experimental groups comprise cells. The cells may comprise primary cells, cell lines, or come in the form of tissue samples. As described above for organisms, the cells in the two experimental groups may differ by a physical characteristic or differ genetically. In a preferred embodiment, the two experimental groups differ in that the cells in one of the experimental groups have been treated with an agent, such as with a compound or drug. In such embodiments, the methods described herein may be used to detect subtle changes that the agent may have on the biomarker set, such as a biochemical or signaling pathway.

X. Nucleic Acid and Polypeptide Agents

In some of embodiments of methods described herein, an agent which reduces the expression of Errα, Gabpa, Gabpb, or any other gene, or an genet used in any of the methods of screening agents described herein, comprises a double stranded RNAi molecule, a ribozyme, or an antisense nucleic acid directed at said gene.

Certain embodiments of the invention make use of materials and methods for effecting knockdown of one form of a gene, by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of in some instances as few as 21 to 22 base pairs in length. Furthermore, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′,5′-AS), which synthesizes a molecule that activates RNAse L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) 3 Biol Chem 254: 10180-3; and Elbashir et al. (2001) Nature 411: 494-8).

RNAi has been shown to be effective in reducing or eliminating the expression of a gene in a number of different organisms including Caenorhabditis elegans (see e.g. Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. For example, if Errα is the target of the double stranded RNA, any of the above RNA species will be designed to include a portion of nucleic acid sequence of the Errα gene.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. Further compositions, methods and applications of RNAi technology are provided in U.S. Pat. Nos. 6,278,039, 5,723,750 and 5,244,805, which are incorporated herein by reference.

Ribozyme molecules designed to catalytically cleave Errα or Gabpa mRNA transcripts can also be used to prevent translation of Errα or Gabpa (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to the gene whose activity is to be reduced.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach (1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell. 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

In addition, ribozymes possess highly specific endoribonuclease activity, which autocatalytically cleaves the target sense mRNA. The present invention extends to ribozymes which hybridize to a sense mRNA encoding a Errα or Gabpa or any other genes of interest described herein, thereby hybridizing to the sense mRNA and cleaving it, such that it is no longer capable of being translated to synthesize a functional polypeptide product.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

In other embodiments of methods described herein, an agent which modulates the activity of Errα, Gabpa, Gabpb, or any other gene, comprises an antibody or fragment thereof. An antibody may increase or decrease the activity of any of the subject polypeptides, and it may increase or decrease the binding of two proteins into a complex, such as an Errα/PCG-1a complex.

Chickens, mammals, such as a mouse, a hamster, a goat, a guinea pig or a rabbit, can be immunized with an immunogenic form of the Errα, Gabpa, Gabpb, or any polypeptide provided by the invention, or with peptide variants thereof (e.g., an antigenic fragment which is capable of eliciting an antibody response). Techniques for conferring immunogenicity on a protein or peptide include conjugation to carriers or other techniques well known in the art. For instance, a peptidyl portion of one of the subject proteins can be administered in the presence of adjuvant. The progress of immunization can be monitored by detection of antibody titers in plasma or serum. Standard ELISA or other immunoassays can be used with the immunogen as antigen to assess the levels of antibodies.

Following immunization, antisera can be obtained and, if desired, polyclonal antibodies against the target protein can be further isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495-497, 1975), as well as the human B cell hybridoma technique (Kozbar et al., Immunology Today, 4: 72, 1983), and the EBV-hybridoma technique to produce human monoclonal antibodies (Cole et al., Monoclonal Antibodies and Cancer Therapy, Alan R. Liss, Inc. pp. 77-96, 1985). Hybridoma cells can be screened immunochemically for production of antibodies specifically reactive to the peptide immunogen and the monoclonal antibodies isolated. Accordingly, another aspect of the invention provides hybridoma cell lines which produce the antibodies described herein. The antibodies can then be tested for their effects on the activity and expression of the protein to which they are directed.

The term antibody as used herein is intended to include fragments which are also specifically reactive with a protein described herein or a complex comprising such protein. Antibodies can be fragmented using conventional techniques and the fragments screened in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules, as well as single chain (scFv) antibodies.

The subject antibodies include trimeric antibodies and humanized antibodies, which can be prepared as described, e.g., in U.S. Pat. No. 5,585,089. Also within the scope of the invention are single chain antibodies. All of these modified forms of antibodies as well as fragments of antibodies are intended to be included in the term “antibody”.

In yet another embodiment of the methods described herein, the agent is a polypeptide, such as an Errα polypeptide or a Gabp polypeptide, or a fragment thereof which retains a biological activity or which antagonizes a biological activity of the wild-type polypeptide. For example, an Errα stimulatory agent comprises an active Errα protein, a nucleic acid molecule encoding Errα that has been introduced into the cell. In another embodiment, the agent is a mutant polypeptide which inhibits Errα protein activity. Examples of such inhibitory agents include a nucleic acid molecule encoding a dominant negative Errα a protein, such a fragment of Errα which may compete with wildtype Errα protein for DNA binding or complex formation with PGC-1.

XI. Therapeutics

In one aspect, the invention provides methods of treating disorders in a subject comprising the administration of a agent or of a composition comprising an agent, such as a therapeutic agent. “Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

In one embodiment, the compositions are pharmaceutical compositions. Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, by aerosol, intravenous, oral or topical route. The administration may comprise intralesional, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

An exemplary composition of the invention comprises an compound capable of modulating the expression or activity of a transcriptional regulator, such as a PGC-1, Gabp or Errα polypeptide, with a delivery system, such as a liposome system, and optionally including an acceptable excipient. In a preferred embodiment, the composition is formulated for injection.

Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, oligomers may be formulated into ointments, salves, gels, or creams as generally known in the art.

Toxicity and therapeutic efficacy of the agents and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of the methods described herein, the effective amount of the agent is between about 1 mg and about 50 mg per kg body weight of the subject. In one embodiment, the effective amount of the agent is between about 2 mg and about 40 mg per kg body weight of the subject. In one embodiment, the effective amount of the agent is between about 3 mg and about 30 mg per kg body weight of the subject. In one embodiment, the effective amount of the agent is between about 4 mg and about 20 mg per kg body weight of the subject. In one embodiment, the effective amount of the agent is between about 5 mg and about 10 mg per kg body weight of the subject.

In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

As for the amount of the compound and/or agent for administration to the subject, one skilled in the art would know how to determine the appropriate amount. As used herein, a dose or amount would be one in sufficient quantities to either inhibit the disorder, treat the disorder, treat the subject or prevent the subject from becoming afflicted with the disorder. This amount may be considered an effective amount. A person of ordinary skill in the art can perform simple titration experiments to determine what amount is required to treat the subject. The dose of the composition of the invention will vary depending on the subject and upon the particular route of administration used. In one embodiment, the dosage can range from about 0.1 to about 100,000 ug/kg body weight of the subject. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

The effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The effective amount will be known to one of skill in the art; it will also be dependent upon the form of the compound, the size of the compound and the bioactivity of the compound. One of skill in the art could routinely perform empirical activity tests for a compound to determine the bioactivity in bioassays and thus determine the effective amount. In one embodiment of the above methods, the effective amount of the compound comprises from about 1.0 ng/kg to about 100 mg/kg body weight of the subject. In another embodiment of the above methods, the effective amount of the compound comprises from about 100 ng/kg to about 50 mg/kg body weight of the subject. In another embodiment of the above methods, the effective amount of the compound comprises from about 1 ug/kg to about 10 mg/kg body weight of the subject. In another embodiment of the above methods, the effective amount of the compound comprises from about 100 ug/kg to about 1 mg/kg body weight of the subject.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, cell types, agents, constructs, or data analysis methods, all without limitation, can be employed, without departing from the scope of the invention as claimed.

The contents of any patents, patent applications, patent publications, or scientific articles referenced anywhere in this application are herein incorporated in their entirety.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

The tables for all the Experimental genes are listed at the end of the third experimental series.

First Experimental Series

Described herein are results of RNA expression profiling of 43 individuals with varying levels of insulin resistance, carried out to systematically identify pathways and processes operative in diabetes. The 43 individuals were: 17 with normal glucose tolerance (NGT), 8 with impaired glucose tolerance (IGT), and 18 with type 2 diabetes (DM2). No single gene showed statistically significant expression differences between the diagnostic classes. Therefore, they developed a new analytical technique, called Gene Set Enrichment Analysis (GSEA), that seeks to determine whether members of gene sets (e.g., pathways) are consistently different, even though modestly or slightly, in one diagnostic class versus another. Application of GSEA to the microarray data, demonstrated that the oxidative phosphorylation pathway (OXPHOS) was significantly different. Of the approximately 106 members in this pathway, 94 are diminished in DM2 versus NGT. The effect is subtle—with each gene only showing a 15-20% decrease.

Also described herein are results of work carried out to define mechanisms underlying this coordinated decrease in expression of OXPHOS genes. Analysis of the expression of these OXPHOS genes in a public atlas of mouse gene expression, showed that ⅔ of all OXPHOS genes are tightly co-regulated across all 47 tissues examined, and that they are highly expressed at the major sites of insulin mediated glucose uptake (brown fat, heart, and skeletal muscle). This group of genes is referred to herein as “ONPHOS-CR,” for “OXPHOS Co-Regulated.” Applicants hypothesized that the transcriptional co-activator PPARGC1 (also known as PGC-1α was responsible for this transcriptional co-regulation. To prove this, Applicants infected mouse muscle cell lines with PPARGC1 and demonstrated that the OXPHOS-CR genes are specifically induced in a time-dependent manner over a three day period. As described in detail below, GSEA was re-applied to the diabetes data, this time testing whether OXPHOS-CR is specifically differentially expressed between the patient classes. Results showed that this accounts for the bulk of the signal detected in the comparison between NGT and DM2, and moreover, appears to be very different between NGT and IGT, as well, suggesting derangements in this group of genes is an early event. Previous studies have suggested that total body aerobic capacity (VO₂max) is predictive of future insulin resistance and diabetes. Interestingly, Applicants found a striking relationship between the mean expression of the OXPHOS-CR genes and total body oxygen consumption.

The following experimental procedures were followed in the first experimental series:

Methods

Human Subjects and Clinical Measurements. Applicants selected 54 men of similar age but with varying degree of glucose tolerance who had been participating in The Malmö Prevention Study in southern Sweden for more than 12 years (Eriksson et al. Diabetologia 33, 526-31. (1990)). The investigation was approved by the Ethics Committée at Lund University, and informed consent was obtained from each of the volunteers. All subjects were Northern Europeans, and their glucose tolerance status was assessed using standardized 75-gram OGTT and by applying WHO85 criteria (Eriksson et al. Diabetologia 33, 526-31. (1990)). At the initial OGTT performed 10 years earlier, none of the men had DM2 (Eriksson et al. Diabetologia 33, 526-31. (1990)). An OGTT performed at the time the biopsy showed that 20 of the subjects had developed manifest type 2 diabetes (DM2), 8 fulfilled the criteria for IGT and 26 had normal glucose tolerance (NGT). As diabetes was diagnosed at the time of the repeat OGTT, none of the subjects were on medication for hyperglycemia or diabetes-related conditions.

Anthropometric and insulin sensitivity measures were performed as previously described (Groop, L. et al. Diabetes 45, 1585-93. (1996)). Height, weight, waist to hip ratio (WHR) and fat free mass were measured on the day of the euglycemic clamp. Maximal oxygen uptake (VO2max) was measured using an incremental work-conducted upright exercise test with a bicycle ergometer (Monark Varberg, Sweden) combined with continuous analysis of expiratory gases and minute ventilation. Exercise was started at a workload varying between 30-100 W depending on the previous history of endurance training or exercise habits and then increased by 20-50 W every 3 min, until a perceived exhaustion or a respiratory quotient of 1.0 was reached. Maximal aerobic capacity was defined as the VO2 during the last 30 s of exercise and is expressed per lean body mass. Insulin sensitivity was determined with a standard 2 hour-euglycemic hyperinsulinemic clamp combined with infusion of tritiated glucose to estimate endogenous glucose production and indirect calorimetry (Deltatrac, Datex Instrumentarium, Finland) to estimate substrate oxidation (Groop, L. et al. Diabetes 45, 1585-93. (1996)). The rate of glucose uptake (also referred to as the M-value) was calculated from the infusion rate of glucose and the residual rate of endogenous glucose production measured by the tritiated glucose tracer during the clamp.

Percutaneous muscle biopsies (20-50 mg) were taken from the vastus lateralis muscle under local anesthesia (1% lidocaine) after the 2-h euglycemic hyperinsulinemic clamp using a Bergström needle (Eriksson et al. Diabetes 43, 805-8. (1994)). Fiber-type composition and glycogen concentration were determined as previously described (Schalin et al. Eur J Clin Invest 25, 693-8. (1995)). Quantification and calculation of the fibers was performed using the COMFAS image analysis system (Scan Beam, Hadsun, Denmark).

Cell Culture and Adenoviral Infection. Mouse myoblasts (C2C12 cells) were cultured and differentiated into myotubes as previously described (Wu, Z. et al. Cell 98, 115-24. (1999)). After 3 days of differentiation, they were infected with an adenovirus containing either green fluorescent protein (GFP) or PGC-1α as previously described (Lin, J. et al. Nature 418, 797-801. (2002)).

mRNA Isolation, Target Preparation, and Hybridization. Targets were prepared from human biopsy or mouse cell lines as previously described (Golub, T. R. et al. Science 286, 531-7. (1999)) and hybridized to the Affymetrix HG-U133A or MG-U74Av2 chip, respectively. Only scans with 10% Present calls and a GAPDH 3′/GAPDH 5′ expression ratio<1.33 were selected. Applicants obtained gene expression data for 54 human samples, but only 43 met these selection criteria; the analysis in this paper is limited to these 43 individuals.

Data Scaling and Filtering. Human microarray data were subjected to global scaling to correct for intensity related biases. For each scan applicants binned all genes according to their expression intensity in a designated reference scan, and recorded the median intensity of that bin to serve as a calibration curve for that scan. Applicants then scaled the expression to the calibration curve of one NGT scan (patient mm12) which applicants visually inspected and deemed high quality using a linear interpolation between the calibration points. Applicants then filtered the 22,283 genes on the HG-U133A chip to eliminate genes that had extremely low expression. A previous study suggested that an Affymetrix average difference level of 100 corresponds to an extremely low level (“not expressed”) (Su, A. I. et al. Proc Natl Acad Sci USA 99, 4465-70. (2002)). Therefore, applicants only considered genes for which there was at least a single measure (average difference) greater than 100. Of the 22,283 genes on the HG-U133A chip, 10,983 genes met this filtering criterion.

Single Gene Microarray Analysis. Microarray analysis to identify individual genes that are significantly different between diagnostic classes was performed using two software packages. First, marker analysis was performed as previously described using GeneCluster. Significance of individual genes was testing by permutation of class labels (5000 iterations), as previously described (Golub, T. R. et al. Science 286, 531-7. (1999)). Applicants used both the t-test and signal to noise difference metrics in these analysis, both yielding comparable results. Second, applicants used the software package SAM, using a A=0.5, to search for gene expression values significantly different between classes (Tusher et al. Proc Natl Acad Sci USA 98, 5116-21. (2001)).

Compilation of Gene Sets. Applicants analyzed 149 gene sets consisting of manually curated pathways and clusters defined by public expression compendia. First, applicants used two different sets of metabolic pathway annotations. Applicants manually curated genes belonging to the following pathways: free fatty acid metabolism, gluconeogenesis, glycolysis, glycogen metabolism, insulin signaling, ketogenesis, pyruvate metabolism, reactive oxygen species (ROS) homeostasis, Kreb's cycle, oxidative phosphorylation (OXPHOS), and mitochondria, using standard textbooks, literature reviews, and LocusLink. Applicants also downloaded NetAFFX (Liu, G. et al et al. Nucleic Acids Res 31, 82-6. (2003)) annotations (October 2002) corresponding to GenMAPP metabolic pathways. To identify sets of co-regulated genes, applicants used self-organizing maps to group the GNF mouse expression atlas into 36 clusters (Su, A. I. et al. Proc Natl Acad Sci USA 99, 4465-70. (2002), Tamayo et al. Proc Natl Acad Sci USA 96, 2907-12. (1999). Genes in these 36 groups were converted to Affymetrix HG-U133A probe sets using the ortholog tables available at the NetAFFX website (October 2002).
Rationale for Grouped Gene Analysis. Consider a microarray dataset with the samples in two categories, A, B. For the sake of simplicity, let the size of A and B each be n. Consider a gene set S for which the expression levels differ between samples of A and B. Model the dataset so that the entry Dᵢⱼ for gene i and sample j is normally distributed with mean μᵢⱼ and standard deviation σ, where

$${μ}_{ij} = \{ \begin{matrix}0 , & i ∉ S \\ + α , & i ∈ S , j ∈ A \\ - α , & i ∈ S , j ∈ B .\end{matrix}$$

Then the signal to noise for an individual gene in S is proportionate to

$$\frac{α \sqrt{n}}{σ} .$$

Suppose on the other hand applicants know S and add the expression levels for all genes in S. Then the signal to noise is proportionate to

$$\frac{α \sqrt{n M}}{σ} ,$$

where M is the number of genes in S. This increases the mean of our statistic (which is standard normal for the null hypothesis of no gene set association) by a factor of \sqrt{M}. If the noise is in fact correlated for genes of S, this reduces the benefit, but applicants can still expect a large gain. In practice applicants will not be able to select a gene set containing fully concordant expression levels, but as long as an appreciable fraction of our gene set exhibits this property, applicants can expect a benefit from the grouped gene approach.
Gene Set Enrichment Analysis (GSEA). GSEA determines if the members of a given gene set are enriched amongst the most differentially expressed genes between two classes. First, the genes are rank ordered on the basis of a difference metric. The results presented in the current experimental series use the signal to noise (SNR) difference metric, which is simply the difference in means of the two classes divided by the sum of the standard deviations of the two diagnostic classes. In general other difference metrics can also be used.

For each gene set, applicants then make an enrichment measure, called the enrichment score (ES), which is a normalized Kolmogorov-Smirnov statistic. Consider the genes R₁, . . . , R_{N} that are rank ordered on the basis of the difference metric between the two classes, and a gene set S containing G members. Applicants define

$$S , {X}_{i} = \sqrt{\frac{N - G}{G}} ,$$

if Rᵢ is not a member of

$${X}_{i} = - \sqrt{\frac{G}{N - G}}$$

if Rᵢ is a member of S. Applicants then compute a running sum across all N genes. The enrichment score (ES) is defined as

$${max}_{1 \leq j \leq N} {\sum }_{i = 1}^{j} {X}_{i} ,$$

or the maximum observed positive deviation of the running sum. ES is measured for every gene set considered. To determine whether any of the given gene sets shows association with the class phenotype distinction, applicants permute the class labels 1000 times, each time recording the maximum ES over all gene sets. Note that in this regard, applicants are testing a single hypothesis. The null hypothesis is that no gene set is associated with the class distinction.

In this experimental series, after identifying OXPHOS-CR as a subset of co-regulated OXPHOS genes, applicants tested it (a single gene set) for association with clinical status using GSEA. Because OXPHOS-CR is not independent of the OXPHOS set interrogated in the initial analysis, this cannot be viewed as an independent hypothesis. For this reason, these P-values are explicitly marked as nominal P-values.

Gene set enrichment analysis (GSEA) has been implemented as a software tool for use with microarray data and will be presented in fuller detail, including a discussion of different varieties of multiple hypothesis testing and applications to other biomedical problems, in a companion paper (Subramanian et. al., in preparation).

Evaluating OXPHOS Coregulation in Mouse Expression Datasets. Applicants used the NetAFFX to identify probe sets on the mouse expression chips corresponding to human OXPHOS probe sets. Applicants identified a total of 114 (106 of which passed our filtering criterion) probe-sets corresponding to the human oxidative phosphorylation genes. Using the October 2002 ortholog tables at NetAFFX, applicants were able to identify 61 mouse orthologs on the Affymetrix MG-U74Av2 chip. Of these 61 probe-sets, 52 were represented in the GNF mouse expression atlas (Su, A. I. et al. Proc Natl Acad Sci USA 99, 4465-70. (2002)). These expression data were normalized to a mean of 0 and a variance of 1. Data were hierarchically clustered and visualized using the Cluster and TreeView software packages (Eisen et al. Proc Natl Acad Sci USA 95, 14863-8. (1998)).

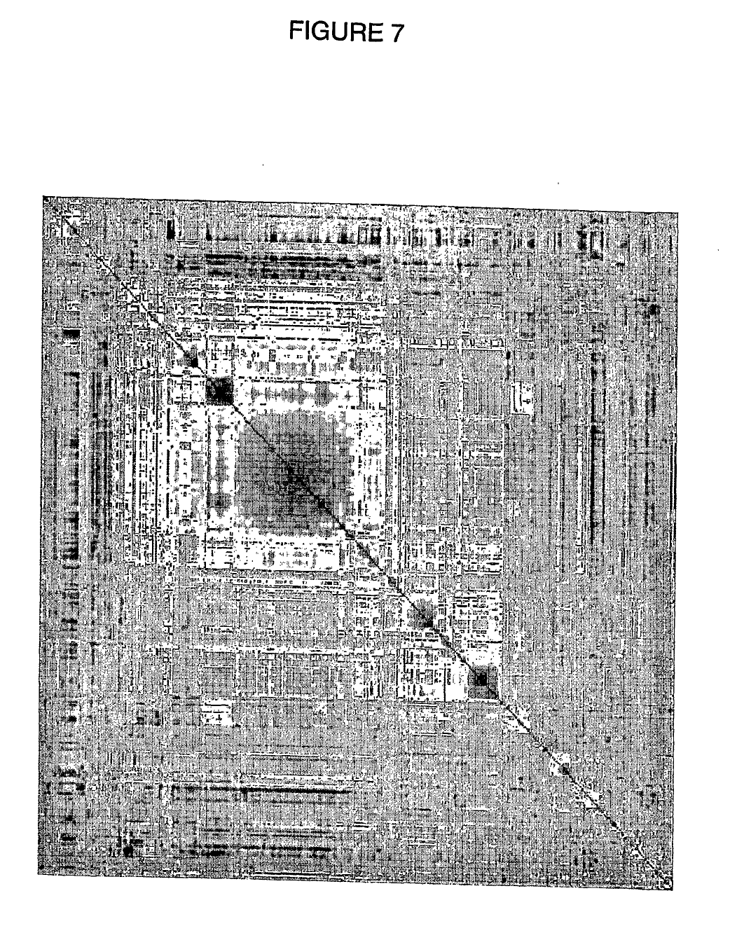

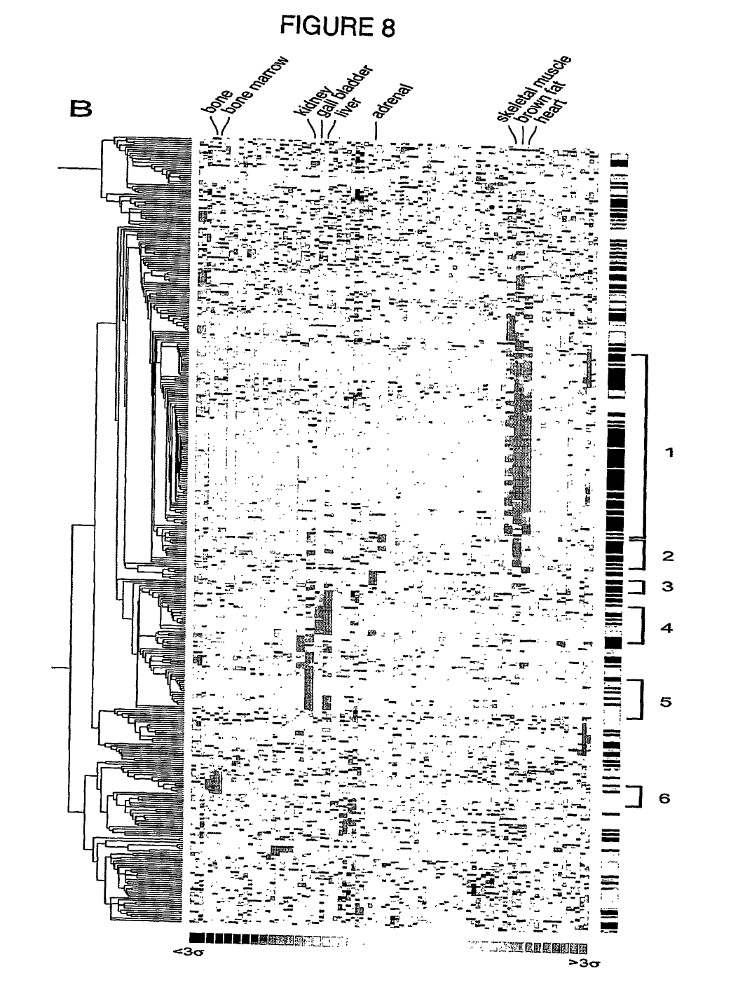

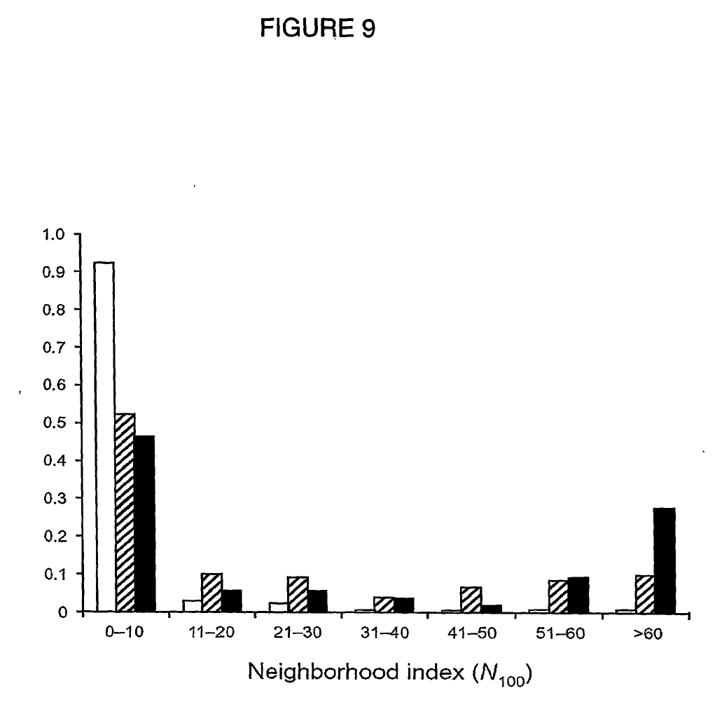

Applicants parsed these 52 genes into 32 co-regulated probe-sets and 20 probe-sets that are not co-regulated, based on the dendrogram in FIGS. 7 and 8. 40 distinct HG-HG-U133A probe-sets mapped to the 32 co-regulated mouse probe-sets, and 19 distinct HG-U133A probe-sets mapped to the 20 mouse probe-sets that are not co-regulated. Five HG-U133A probe-sets are shared between these two groups, representing ambiguous cases (i.e., these human probe-sets that map to two mouse probe-sets, one of which is co-regulated and the other of which is not co-regulated). Applicants discarded these five ambiguous human probe-sets from our analysis. This left a total of 35 HG-U133A probe-sets which applicants call OXPHOS-CR genes, and a total of 14 HG-U133A probe-sets which applicants call OXPHOS not CR. Note that 34 and 13 of these genes, respectively, passed our filtering criteria, and these were the genes used in FIG. 9 as well as in the OXPHOS-CR analysis described in the paper.

Linear Regression Analysis. Applicants generated linear regression models using SAS (SAS Institute, USA). Clinical variables were used as dependent variables, and OXPHOS-CR gene expression levels or other clinical/biochemical measures used as the independent (explanatory or predictor) variables. To compute the mean centroid of OXPHOS-CR, the 34 genes OXPHOS-CR gene expression levels were normalized to a mean 0 and a variance 1 across all 43 patients. The OXPHOS-CR mean centroid vector is simply the mean of these 34 expression vectors. In some regression analyses, applicants introduced dummy variables to represent diabetes status. For the regressions applicants have performed, applicants have reported the adjusted squared correlation coefficient (R²_{adj}), which corrects for the degrees of freedom.

### Example 1

### Comparison of Gene Expression in between Experimental Groups

DNA microarrays were used to profile expression of over 22,000 genes in skeletal muscle biopsies from 43 age-matched males (Table 1): 17 with Normal Glucose Tolerance (NGT), 8 with Impaired Glucose Tolerance (IGT), and 18 with Type 2 Diabetes Mellitus (DM2). Biopsies were obtained at the time of diagnosis (before treatment with hypoglycemic medication) and under the controlled conditions of a hyperinsulinemic euglycemic clamp (see Methods). When assessed with either of two different analytical techniques (Golub, T. R. et al. Science 286, 531-7. (1999), Tusher et al. Proc Natl Acad Sci USA 98, 5116-21. (2001)) that take into account the multiple comparisons implicit in microarray analysis, no single gene exhibited a significant difference in expression between the diagnostic categories. This result is consistent with smaller studies (Sreekumar et al. Diabetes 51, 1913-20. (2002), Yang et al. Diabetologia 45, 1584-93. (2002)) which failed to identify any individual gene whose expression difference was significant when corrected for the large number of hypotheses tested (Kropf et al. Biometrical J. 44, 789-800 (2002), Storey et al. J. R. Statist. Soc. B 64, 479-498 (2002)).

### Example 2

### Gene Set Enrichment Analysis

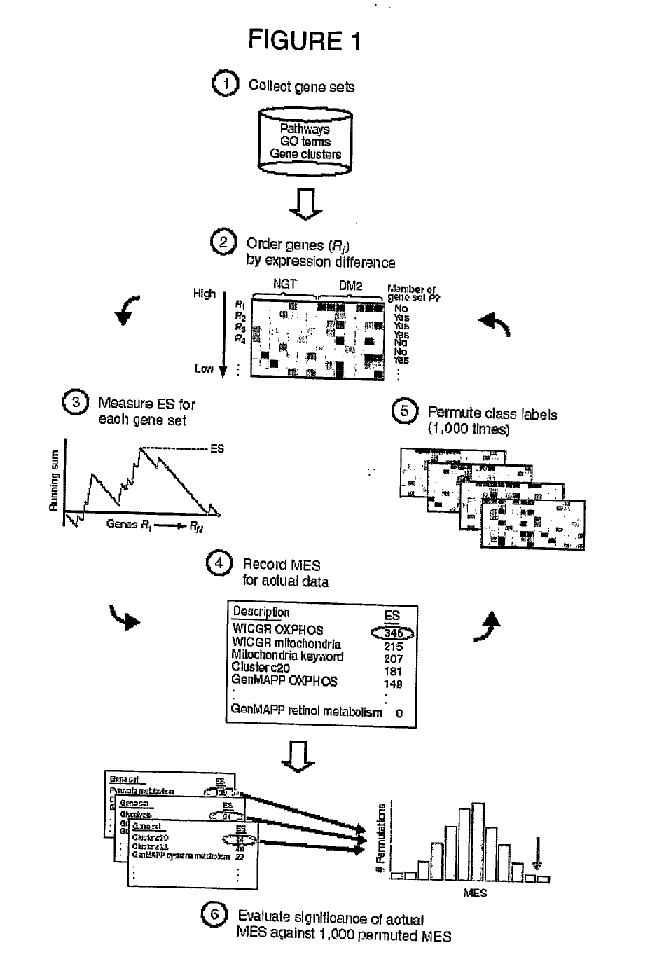

To test for sets of related genes that might be systematically altered in diabetic muscle, Applicants devised a simple approach called Gene Set Enrichment Analysis (GSEA), which is introduced here (see FIG. 1 and Methods). The method combines information from the members of previously defined sets of genes (e.g., biological pathways) to increase signal relative to noise (see Methods) and improve statistical power.

For a given pairwise comparison (e.g., high in NGT vs DM2), all genes are ranked based on the difference in expression (using an appropriate metric such as signal to noise). The null hypothesis of GSEA is that the rank ordering of the genes in a given comparison is random with regard to the diagnostic categorization of the samples. The alternative hypothesis is the rank ordering of the pathway members is associated with the specific diagnostic criteria used to categorize the patient groups.

The extent of association is then measured by a non-parametric, running sum statistic termed the enrichment score (ES), and record the Maximum ES (MES) over all gene sets in the actual patient data (FIG. 1). To assess the statistical significance of the MES, applicants use permutation testing of the patient diagnostic labels (for example, whether a patient is NGT or DM2, see FIG. 1). Specifically, applicants compare the MES achieved in the actual data to that seen in each of 1,000 permutations that shuffled the diagnostic labels among the samples. The significance of the MES score is calculated as the fraction of the 1,000 random permutations in which the top pathway gave a stronger result than that observed in the actual data. Because the permutation test involves randomization of the patient labels, it is a test for the dependence on the actual diagnostic status of the patients. Moreover, because the actual MES is compared to the distribution of maximal ES values over all pathways examined in each of the randomized datasets, it accounts for multiple pathways tested, and no further correction is required (Kropf et al. Biometrical J. 44, 789-800 (2002), Storey et al. J. R. Statist. Soc. B 64, 479-498 (2002).

### Example 3

### Decreased Expression of Genes Involved in Oxidative Phosphorylation

Applicants applied GSEA to the microarray data described above, using 149 gene sets that applicants compiled (Table 2). Of these gene sets, 113 are based on involvement in metabolic pathways (based on public or local curation (Liu, G. et al et al. Nucleic Acids Res 31, 82-6. (2003)) and 36 consist of gene clusters that exhibit co-regulation in a mouse expression atlas of 46 tissues (Su, A. I. et al. Proc Natl Acad Sci USA 99, 4465-70. (2002)) (see Methods). The gene sets were selected without regard to the results of the microarray data from our patients. The top gene set in GSEA analysis yielded a Maximal Enrichment Score (MES=346) that was significant at P=0.029 over the 1,000 permutations of the 149 pathways. That is, in only 29 or 1,000 permutations did the top pathway (of the 149) exceed the score achieved by the top pathway achieved using the actual diagnostic labels.

The maximal ES score was obtained for an internally curated set consisting of genes involved in oxidative phosphorylation (applicants refer to this gene set as OXPHOS). Interestingly, the four gene sets with the next highest ES scores overlap with this OXPHOS gene set, and their enrichment is almost entirely explained by the overlap: a locally curated set of genes involved in mitochondrial function, a set of genes identified with the keyword ‘mitochondria,’ a cluster (referred to here as c20) of co-regulated genes derived from the comparison of publicly available mouse data, and a set of genes related to oxidative phosphorylation defined at the Affymetrix website (Liu, G. et al et al. Nucleic Acids Res 31, 82-6. (2003)).

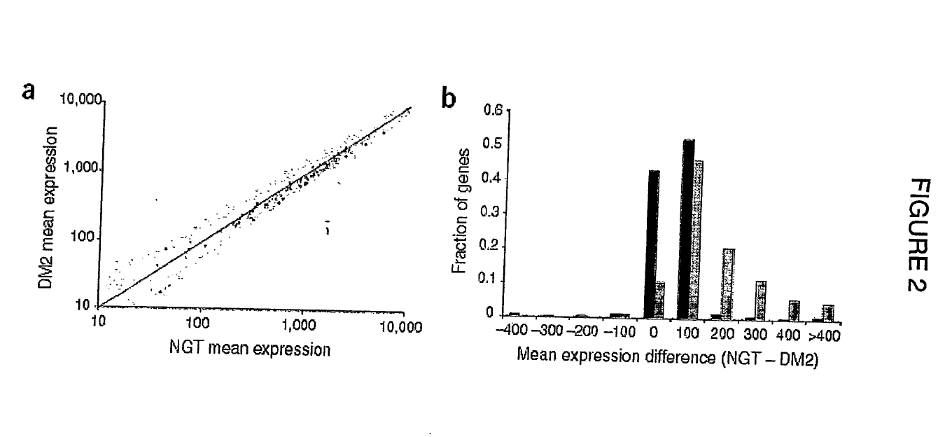

Examination of the individual expression values for the 106 OXPHOS genes reveals the source of this signal (FIG. 2). Although the typical decrease in expression for individual OXPHOS genes is very modest (˜20%), the decrease is remarkably consistent across the set: 89% (94 of 106) of the genes showing decreased expression in DM2 relative to NGT (FIG. 2). As controls, applicants confirmed that the result is independent of specific aspects of data processing (such as scaling, thresholding, filtering) or of selection of difference metrics. Moreover, the result identified by GSEA is supported by previous observations: others have shown that oxidative capacities are altered in insulin resistant muscle (Bjorntorp, et al. Diabetologia 3, 346-52. (1967), Simoneau et al. Faseb J 9, 273-8. (1995), and recent microarray analyses of human diabetic muscle have identified genes in oxidative phosphorylation among their top-ranked genes (Sreekumar et al. Diabetes 51, 1913-20. (2002)).

### Example 4

### OXPHOS-CR: A Coregulated Subset of OXPHOS Genes

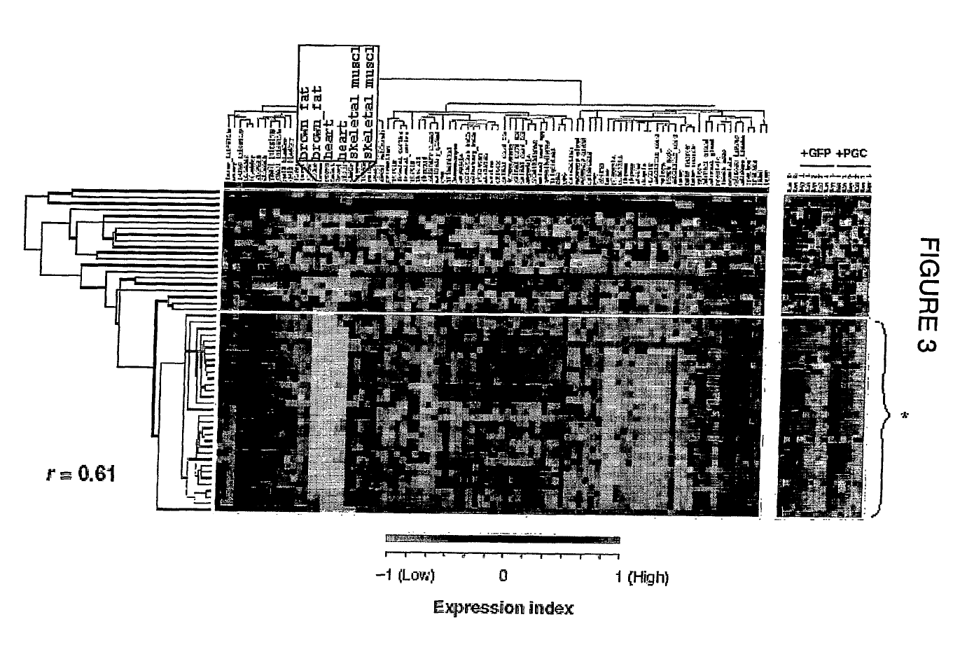

One of the overlapping gene sets identified by GSEA is cluster c20, defined as a set of genes that are tightly co-regulated across many tissues (see Methods). The partial overlap of OXPHOS with the coregulated cluster led us to ask whether all OXPHOS genes are coordinately regulated, or just a subset. Applicants examined transcriptional co-regulation of mouse homologs of OXPHOS genes across a mouse tissue expression atlas (Su, A. I. et al. Proc Natl Acad Sci USA 99, 4465-70. (2002)). This revealed a previously unrecognized subset of the OXPHOS biochemical pathway, corresponding to about two-thirds of the OXPHOS genes, that exhibit strong correlation across mouse tissues (r=0.67) (FIG. 3a). Applicants term this subset OXPHOS-CR (OXidative PHOSphorylation Co-Regulated). The remaining OXPHOS genes show little co-regulation with OXHPOS-CR or each other (FIG. 3a). The OXPHOS-CR subset strongly expressed in three of 46 tissues: skeletal muscle, heart, and brown fat. Applicants note that these are the major sites of insulin-mediated glucose disposal in mice.

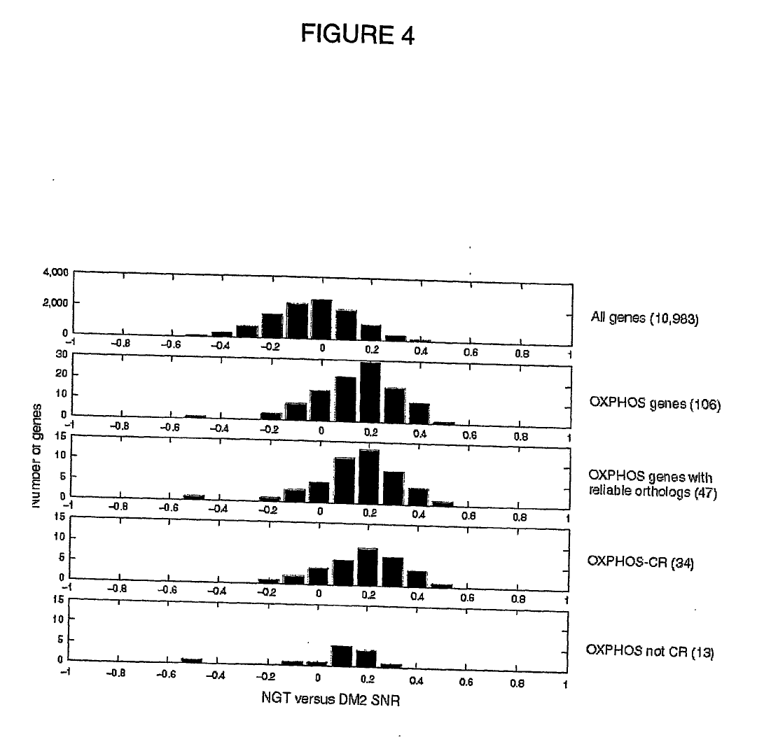

Applicants next asked whether the downregulation of OXPHOS observed in DM2 was a general property of all OXPHOS genes or was specific to OXPHOS-CR. Interestingly, the bulk of the statistical signal applicants observe in GSEA is accounted for by OXPHOS-CR (FIG. 4). Namely, the OXPHOS-CR subset showed a stronger mean deviation than the remainder of the OXPHOS gene set (FIG. 4), and was itself significant in the GSEA analysis (nominal P-value 0.001, as compared to nominal P=0.226 for the remainder of the OXPHOS set). To see if these changes were secondary to hyperglycemia per se, or preceded the onset of frank diabetes, applicants compared expression of OXPHOS-CR in NGT patients to those with the pre-diabetic state, IGT. Applicants found that expression of OXPHOS-CR is also downregulated in IGT (nominal P<10⁻⁴). This suggests that downregulation of OXPHOS-CR precedes onset of hyperglycemia. Thus, GSEA allowed us to detect a subset of OXPHOS genes, called OXPHOS-CR, with three key properties: (1) they are members of the oxidative phosphorylation pathway, (2) they are tightly co-regulated across many tissues and are highly expressed in the major sites of insulin mediated glucose disposal, and (3) they exhibit a subtle but consistent decreased expression in muscle from patients with both the pre-diabetic state IGT and type 2 diabetes.

### Example 5

### PGC-1α can Induce Expression of OXPHOS-CR

The strong correlation in expression of the OXPHOS-CR genes and their coordinated downregulation in diabetic muscle led us to explore mechanisms that might mediate to this tight control. Applicants reasoned that peroxisome proliferator-activated receptor γ coactivator 1 [symbol] (PGC-1α), a cold-inducible regulator of mitochondrial biogenesis, thermogenesis, and skeletal muscle fiber type switching (Puigserver, P. et al. Cell 92, 829-39. (1998), Wu, Z. et al. Cell 98, 115-24. (1999), Lin, J. et al. Nature 418, 797-801. (2002)), was a prime candidate for mediating these effects. Consistent with this hypothesis, applicants observed that mean levels of PGC-1α transcript were similarly decreased (−20%) in the diabetic muscle, and noted that the promoters of several of the OXPHOS-CR genes have been reported to contain binding sites for nuclear respiratory factor 1, a transcription factor co-activated by PGC-1α (Scarpulla, R. C. Biochim Biophys Acta 1576, 1-14. (2002)).

To test directly whether OXPHOS-CR genes might be transcriptional targets of PGC-1α, applicants expressed PGC-1α in a mouse skeletal muscle cell line using an adenoviral expression vector (Lin, J. et al. Nature 418, 797-801. (2002)) and used DNA microarrays to profile expression of the OXPHOS genes over a 3 day period (see Methods). Applicants found that a subset of OXPHOS genes were strongly upregulated in a time-dependent manner in response to PGC-1α, and that this subset corresponds almost precisely to OXPHOS-CR (FIG. 3b). These in vitro results support the hypothesis that PGC-1 plays a role in the regulation of OXPHOS-CR, both across the mouse tissue compendium as well as in the observed downregulation in diabetes.

### Example 6

### Expression of OXPHOS-CR and Measures of Whole Body Physiology

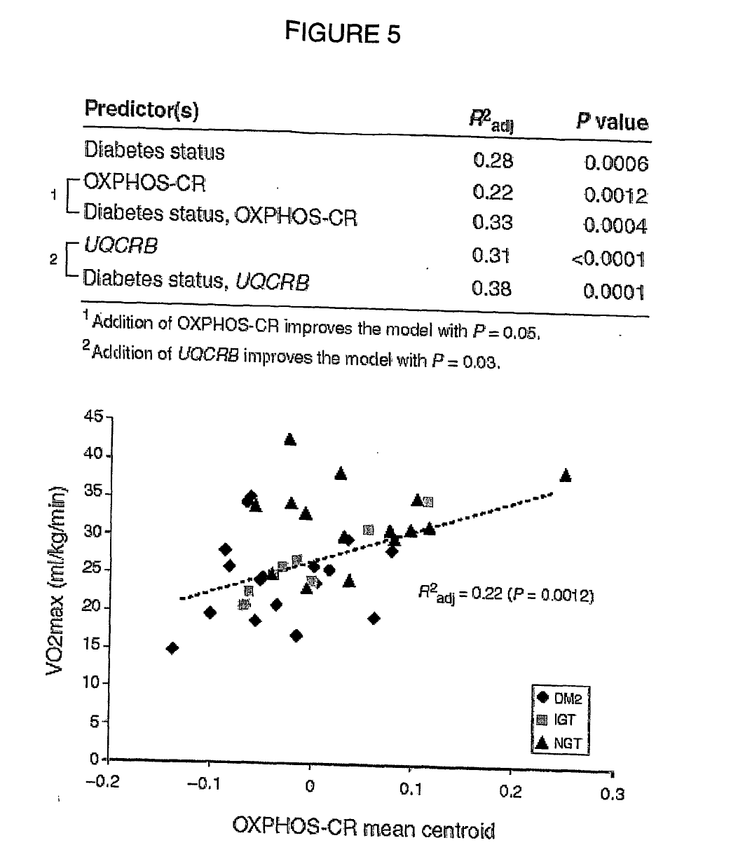

Metabolic control theory suggests that small increases in many sequential steps of a metabolic pathway can lead to a dramatic change in the total flux through the pathway, whereas large changes in a single enzyme might have no measurable effects (Brown et al. Biochem J 284, 1-13. (1992). To test the hypothesis that subtle differences in OXPHOS-CR gene expression in diabetic patients might be related to changes in total body metabolism, applicants examined the relationships between diabetes status, expression of OXPHOS-CR genes, and VO2max as measured in our patients (FIG. 5). Consistent with previous reports (Eriksson et al. Diabetologia 33, 526-31. (1990)), diabetes and VO2max are correlated in our patients (R_{adj}²=0.28, P=0.0005). Strikingly, applicants found that the expression of OXPHOS-CR genes in muscle is strongly correlated with VO2max (R_{adj}²=0.22, P=0.0012) (FIG. 5), a measure of total-body physiology. The top ranking OXPHOS-CR gene, ubiquinol cytochrome c reductase binding protein (UQCRB), is even a stronger predictor (R_{adj}²=0.31, P<0.0001). OXPHOS-CR appears to be not solely a proxy for diabetes status, however, because a two-variable regression of VO2max on diabetes status and OXPHOS-CR expression level shows that both variables contribute significantly to the correlation (P=0.05 for the model with both variables as compared to the model with only diabetes status).

It is important to note that these results do not seem secondary to other known predictors of oxidative capacity. Applicants found no relationship between BMI or WHR and OXPHOS-CR gene expression (R_{adj}²<0.01 in both cases). In addition, there was no significant relationship between quantitative measures of fiber types and OXPHOS-CR expression. Thus, subtle decrease in expression of OXPHOS-CR genes in muscle appears to be associated with changes in total body aerobic capacity, even beyond their correlation to diabetes status, body habitus, or muscle fiber type.

Second Experimental Series

The following experimental procedures were followed in the second experimental series:

Organelle Purification and Sample Preparation. 6-8 week old male mice were subjected to an 8 hour fast and then euthanized. Brain, heart, kidney, and livers were harvested immediately and placed in ice cold saline. Mitochondria were isolated using differential centrifugation as previously described and purified with a Percoll gradient (Mootha et al. (2003). Proc Natl Acad Sci USA 100, 605-10). The proteins were then solubilized, size separated, and digested as previously described (Mootha et al. (2003). Proc Natl Acad Sci USA 100, 605-10)).

Tandem Mass Spectrometry. Liquid chromatography tandem mass spectrometry (LC-MS/MS) was performed on QSTAR pulsar quadrupole time of flight mass spectrometers (AB/MDS Sciex, Toronto) as described previously (Mootha et al. (2003). Proc Natl Acad Sci USA 100, 605-10). Tandem mass spectra were searched against the NCBInr database (February 2002) with tryptic constraints and initial mass tolerances<0.13 Da in the search software Mascot (Matrix Sciences, London). Only peptides achieving a Mascot score above 25 and containing a sequence tag of at least three consecutive amino acids were accepted.

Curation of Previously Annotated Mitochondrial Proteins. Two key sources were used to identify previously annotated proteins. First, Applicant downloaded the 308 human and 117 mouse protein sequences at MITOcondria Project (Scharfe et al. (2000). Nucleic Acids Res 28, 155-8). Applicant also downloaded the 199 human and 290 mouse protein sequences annotated at LocusLink (http://www.ncbi.nlm.nih.gov/LocusLink) as having a mitochondrial subcellular localization based on gene ontology terminology (GO:0005739) (Lewis et al. (2000). Curr Opin Struct Biol 10, 349-54) (January 2003). Also included in the master list the are 13 mtDNA encoded proteins, based on LocusLink annotation.

A Nonredundant List of Mitochondrial Proteins. FASTA sequences corresponding to the previously annotated mitochondrial proteins, newly identified mitochondrial proteins, and the mouse Reference Sequences (Maglott et al. (2000). Nucleic Acids Res 28, 126-8) were merged. These were then collapsed into distinct protein clusters using a downloaded version of blastclust (http://www.ncbi.nlm.nih.gov/BLAST/). Applicants required that members of a cluster demonstrate 70% sequence identity over 50% of the total length, not requiring a reciprocal relationship to exist. Clusters containing multiple Reference Sequences were then broken using a higher stringency blastclust, in which applicants required 90% identity over 50% of the length. Clusters containing hemoglobin, trypsin, and albumin were eliminated as obvious contaminants. When possible the Reference Sequence was selected as the exemplar from the cluster, otherwise another sequence was manually selected. Hence, each cluster is annotated by an exemplar sequence, the protein accessions (and tissues) in which the proteins were found in the proteomics experiments, and the protein accessions corresponding to annotation sources. Applicant obtained a total of 612 distinct protein clusters (Table 2). The GenPept descriptions of 37 of these exemplars suggested that they are mitochondrial, but simply missed by the automated annotation procedure using the MITOP and LocusLink databases. These exemplars were therefore manually annotated as previously known mitochondrial proteins, to provide a more conservative estimate of our sensitivity measure and newly discovered proteins.

Statistical Analysis. Cluster enrichment was determined using a cumulative hypergeometric distribution. To determine whether two empirical cumulative distributions arise from the same underlying distribution, Applicant used the Kolmogorov-Smirnov test statistic, D. Tail values were obtained using Matlab (Mathworks).
RNA/Protein Concordance Test. the RNA/protein concordance test was developed to determine whether there is significant concordance between protein detection in a proteomics experiment and mRNA abundance in a microarray experiment. Consider the pair of tissues, i,j, where i,jε{brain, heart, kidney, liver}. For a given gene, G, let M(G,k) represent the gene expression level of gene G in tissue k. Let P(G,k) be an indicator variable that is 0 if the protein product of gene G is not found in tissue k, and 1 if the protein product is found in tissue k. The mRNA and protein expression levels of gene G are concordant in tissues i and j if M(G,i)>M(G,j) when P(G,i)>P(G,j). For a given gene, G, compute the total number of observed concordances (c_{G}) between all pairs of tissues as well as the expected variance in concordance (v_{G}) for that gene. The test statistic is simply

$$C = \frac{{\sum }_{G} {c}_{G}}{\sqrt{{\sum }_{G} {v}_{G}}} ,$$

which has mean 0 and variance 1 and is approximately normal in the null case where there is no concordance between RNA abundance and protein detection.
Compositional Diversity Across Tissues. Mitochondrial gene products show distinct patterns of expression based on protein and RNA expression (Table 5). These patterns of distribution can be used to develop a simple model that describes core mitochondrial proteins versus those that are specialized to any set of cell types.

Consider a set of i+1 tissues, Sᵢ₊₁, as well as a distinct subset Sᵢ, i.e., Sᵢ⊂Sᵢ₊₁, where i>0. Applicants are interested in the probability that a given gene product is found in Sᵢ₊₁ conditional that it is found in Sᵢ, or simply T(Sᵢ₊₁, Sᵢ)=P (gene product is found in Sᵢ₊₁|gene product is found in Sᵢ). Define Pᵢ as the average T(Sᵢ₊₁, Sᵢ) over all selections of Sᵢ⊂Sᵢ₊₁. When applicant assessed compositional diversity using RNA expression levels, Applicant interpreted an RNA expression level greater than 200 as present (Su et al. (2002). Proc Natl Acad Sci USA 99, 4465-70), and an expression below this level as not present. These average conditional probabilities Pᵢ can also be modeled. Imagine that a fraction f of all mitochondrial proteins are ubiquitous (i.e., expressed in all cell types with probability 1) and that a fraction 1−f are not ubiquitous, but rather, appear in a given tissue with probability p. Then Pᵢ₊₁=(f+(1−f)pⁱ⁺¹)/(f+(1−f)pⁱ).

DNA Microarray Analysis. To identify Affymetrix probe-sets corresponding to each protein cluster, Applicant mapped the exemplar sequence to the Unigene cluster, and then identified the corresponding Affymetrix MG-U74Av2 probe set. The NetAffx website (http://www.affymetrix.com) and its tables were used to perform these mappings (January 2003). The GNF mouse expression atlas (Su et al. (2002). Proc Natl Acad Sci USA 99, 4465-70) was downloaded from its website (http://www.gnf.org). In comparisons of protein detection and mRNA abundance, the used the mRNA expression level for a given tissue averaged over the replicates, since the GNF mouse expression atlas includes duplicates for each tissue. Because the proteomic survey was performed on whole brain, applicants simply compared to the average expression of all brain samples in the GNF mouse atlas. Hierarchical clustering was performed using DCHIP (Schadt et al. (2001). J Cell Biochem Suppl Suppl, 120-5).

Identification of Ancestral Mitochondrial Genes. The consensus FASTA sequences for the genes represented on the Affymetrix MG-U74Av2 oligonucleotide array were downloaded from the NetAFFX (Liu et al. (2003). Nucleic Acids Res 31, 82-6) website (http://www.affymetrix.com). A blastx comparison of these sequences was performed against the Rickettsia prowazekii protein sequences, downloaded from the NCBI, and then a tblastn comparison of the bacterial protein sequences was performed against the consensus FASTA sequences. An ancestral gene as defined as one achieving a BLASTX E<0.01 and having a reciprocal best match in the BLAST analysis.

### Example 7

### Proteomic Survey of Mitochondria

Applicants carried out a systematic survey of mitochondrial proteins from brain, heart, kidney, and liver of C57BL6/S mice (see Methods). Each of these tissues provides a rich source of mitochondria. The isolation consisted of density centrifugation followed by Percoll purification. Preparations were tested for purity and for contamination using immunoblotting directed against organelle markers, enzymatic assays to ensure that the mitochondria were intact, and electron microscopy. The liver, heart, and kidney mitochondria were extremely pure. The brain mitochondria tended to show persistent contamination by synaptosomes, which themselves are a rich source of neuronal mitochondria (see Fernandez-Vizarra (2002). Methods 26, 292-7).

Mitochondrial proteins from each tissue were solubilized and size separated by gel filtration chromatography into approximately 20 fractions (see Methods). These proteins were then digested and analyzed by liquid chromatography mass spectrometry/mass spectrometry (LC-MS/MS). More than 100 LC-MS/MS experiments were performed (see Methods).

The acquired tandem mass spectra were then searched against the NCBI nonredundant database consisting of mammalian proteins using a probability-based method (Perkins et al. (1999). Electrophoresis 20, 3551-67. [pii]). Stringent criteria were used for accepting a database hit. Specifically, only peptides corresponding to complete tryptic cleavage specificity with scores greater than 25 were considered (see Methods). Furthermore, only fragmentation spectra which also exhibited a correct, corresponding peptide sequence tag (Mann et al. (1994). Anal Chem 66, 4390-9) consisting of at least three amino acids were considered.

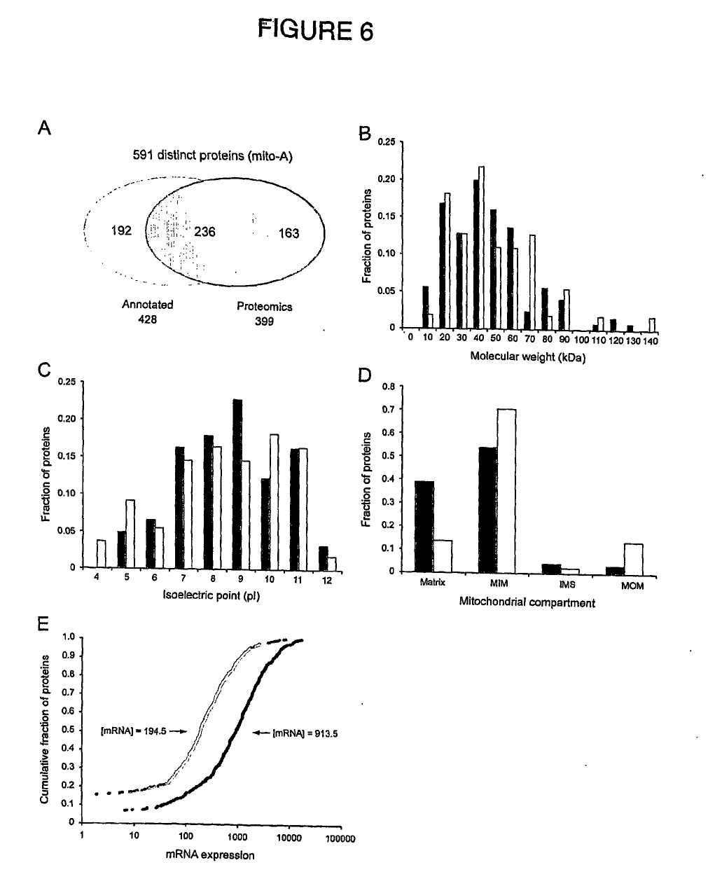

Using these criteria, ˜2100 database hits were identified. This list contains a high degree of redundancy, because a protein may have been found in adjacent fractions of the gel and in different tissues. The ˜2100 hits collapse to a distinct set of 422 mouse proteins (see Table 4, FIG. 6, and Methods).

### Example 8

### Previously Annotated Mitochondrial Proteins

A list of previously annotated mouse and human mitochondrial proteins was created by pooling all the mouse and human proteins from MITOchondria Project (MITOP, http://mips.gsf.de/proj/medgen/mitop/), a public database of curated mitochondrial proteins, as well as all proteins annotated as mitochondrial in NCBI's LocusLink database (http://www.ncbi.nlm.nih.gov/LocusLink/) (see Methods). After elimination of redundancy, the list contains 452 distinct mouse proteins that are either directly annotated as mitochondrial or whose human homolog is annotated as mitochondrial (FIG. 6A). The human proteins recently reported to be mitochondrial by Taylor et. al. 2003 (in a study published after the construction of Applicant's list of previously annotated proteins) were not included in Applicant's list. These proteins instead serve as a control against which to compare the proteins identified in our proteomic analysis. The list of 452 previously annotated mitochondrial proteins is by no means comprehensive—there are likely many mitochondrial proteins that are simply not annotated by these public databases. However, it does provide a reasonable, high confidence list of previously annotated proteins against which to benchmark Applicant's proteomic survey.

### Example 9

### Newly Identified Mitochondrial Proteins

The set of 422 proteins identified in Applicant's proteomic survey include 262 of the 452 proteins previously annotated to be mitochondrial (58%) and 160 proteins not previously annotated as associated with the mitochondria (FIG. 6A). The previous and new sets were combined to produce a list of 612 genes whose protein product is physically associated with mitochondria. This set of genes is referred to as mito-P (Table 4).

The 422 proteins identified in the proteomic survey span a wide range of isoelectric points and molecular weights (FIG. 6B, 6C), although proteins from the inner mitochondrial membrane are underrepresented (FIG. 6D). The incomplete sensitivity (58%) is most likely due to a bias against proteins of low abundance, which is a known feature of the mass spectrometry methodology. This explanation is supported by analysis of RNA expression of the genes encoding the detected and undetected proteins. Considering the subset of the 452 previously annotated genes for which RNA expression was reported in a recent atlas of mRNA expression in mouse 0, the distribution of RNA expression level was about 5-fold higher for the genes whose products were detected in our proteomic survey as compared to those that were not (P=1×10⁻²¹) (FIG. 6E). This suggests that the proteomics strategy preferentially detected the higher abundance proteins

The 160 proteins not previously annotated as mitochondrial potentially represent new mitochondrial proteins, either in the conventional sense of being present within the organelle or in a broader sense of being tethered to the mitochondrial outer membrane (e.g., tubulin (Heggeness et al. (1978). Proc Natl Acad Sci USA 75, 3863-6)).

To test this notion, Applicants sought independent evidence that these 160 proteins are actually mitochondrial. First, the list was compared to proteins identified in a recent survey of human heart mitochondria (Taylor et al. (2003). Nat Biotechnol 18, 18). Human homologs of 64 of the 160 proteins were identified in this recently published study. Of the remaining 96 proteins, 24 have strong mitochondrial targeting sequences based on bioinformatic analysis of protein targeting sequences (Table 4 and Methods) (Nakai et al. (1999). Trends Biochem Sci 24, 34-6), a proportion similar to the known mitochondrial proteins. For example polymerase delta interacting protein 38 (encoded by Pdip38-pending), which was detected only in liver mitochondria, and the gene product of Rnaseh1, which was found only in the kidney, have strong mitochondrial targeting scores. A recent study confirmed that Rnaseh1 can be localized to the mitochondrion, where it plays a critical role in mtDNA homeostasis (Cerritelli et al. (2003). Mol Cell 11, 807-15).

### Example 10

### Modules of Coregulated Mitochondrial Genes

Applicant also investigated co-regulation of the 612 mito-P genes across different tissues. For 388 of the 612 mito-P genes, mRNA expression levels were available in a mouse gene expression compendium containing data across 47 tissues (Su et al. (2002). Proc Natl Acad Sci USA 99, 4465-70).

Applicant calculated pairwise correlation and performed hierarchical clustering of these 388 gene expression profiles (FIGS. 6 and 7). There are several striking mitochondrial gene modules (FIG. 6), which are defined here as clusters of genes showing strong expression correlation across the 47 tissues (Table 6). These modules include genes with strong annotation support as well as genes identified in this study as being mitochondrial (see bar labeling in FIG. 7). These clusters appear to have properties of scale-free networks, in which a few central nodes are highly correlated with each other (module 6), while most are correlated with only a few genes or none at all (Barabasi, (2003). Scale-free networks, Sci Am 288, 60-9). As shown in FIG. 7, mitochondrial gene expression profiles vary tremendously from tissue to tissue, consistent with the compositional diversity of mitochondria noted above.

Some of these gene modules have no obvious functional relationships, though two appear to be enriched in certain tissues (modules 1,2). Each of these gene modules is characterized by tightly correlated gene expression across the tissue compendium. Members of these genes likely share transcriptional regulatory mechanisms as well as cellular functions. Many of the newly identified mitochondrial genes (black bar in annotation bar of FIG. 7) lie within these modules, providing a functional context for their cellular role.

The mitochondria gene modules provide an initial step towards the characterization of some of the newly identified mitochondrial genes, since functionally related genes tend to have correlated gene expression. Of the 104 newly identified mitochondrial proteins that are represented in this microarray dataset, 38 fall within these 7 modules, providing them with a preliminary functional context.

### Example 11

### Modules Enriched in Genes of Oxidative Phosphorylation

A striking gene module (module 6) consists of genes related to oxidative phosphorylation (OXPHOS) and β-oxidation and expressed at high levels in brown fat, skeletal muscle, and heart (FIGS. 6 and 7). The related module 5, enriched in OXPHOS genes but not the β-oxidation genes, is expressed not only in brown fat, heart, and skeletal muscle, but also in colon. Colon is not traditionally considered to be a highly metabolic tissue, but it has high expression of peroxisome proliferative activated receptor-γ, a partner of PGC-1α, a master regulator of mitochondrial biogenesis (Puigserver et al. (2003). Endocr Rev 24, 78-90). In a recent study of human diabetic muscle, Applicant and co-workers demonstrated that the OXPHOS genes in modules 5 and 6 (termed OXPHOS-CR for OXidative PHOShorylation CoRegulated) show diminished expression in type 2 diabetes, and that these genes are targets of PGC-1α. The current study identifies two modules (modules 5, 6) that contain OXPHOS-CR as well as other mitochondrial genes, including 4 newly identified genes in module 5 and 12 newly mitochondrial genes in module 6. It will be interesting to determine how this expanded set contributes to type 2 diabetes and other measures of whole-body metabolism.

### Example 12

### Mitochondrial Gene Expression Neighborhood

Applicant also sought to systematically identify all genes that exhibit correlated expression with the mito-P genes. This was done using the neighborhood index (N₁₀₀), a previously described statistic that measures a given gene's expression similarity to a target gene set (Mootha et al. (2003). Proc Natl Acad Sci USA 100, 605-10). For a given gene, the mitochondria neighborhood index is defined as the number of mito-P genes among its nearest 100 expression neighbors. Applicant computed the N₁₀₀ statistic for all genes in the mouse expression atlas (FIG. 9).

The 10,043 genes in the mouse expression atlas include 388 of the 612 mito-P genes. If these 388 genes were a random subset, an N₁₀₀ value greater than 10 would be expected to occur by chance 1 in 1000 times, and an N₁₀₀ greater than 50 would be exceedingly rare (P=1.5×10⁻¹⁴).

A total of 806 genes have N₁₀₀>10. This is defined herein as the expression neighborhood of the mito-P set, and Applicant interprets these genes as being co-regulated with mitochondrial genes (see the entire rank ordered list, Table 7). This group corresponds to only 8% of all the genes studied, but it contains 52% of the mito-P genes (6.5-fold enrichment, P=1.49×10⁻¹¹). The list includes 59 that are newly mitochondrial, based on the proteomic survey described herein and 25 that were previously known to be mitochondrial but not detected by that proteomic survey.

Importantly, the expression neighborhood includes 605 genes not present in the mito-P set itself. These genes may encode proteins that are physically present in mitochondria but were missed in the proteomic survey or that are functionally related to mitochondria but not physically associated. They provide a catalog of genes that are likely functionally relevant to mitochondrial biology, and are complementary to the proteomic approach that identified proteins resident in this organelle.

### Example 13

### Transcription Factors and Nutrient Sensors within the Mitochondrial Neighborhood

Applicant found several genes involved in DNA replication within the mitochondria neighborhood (Table 1). Essra, Pparg, and Ppara encode nuclear receptors that are tightly co-regulated with the mitochondrial genes. This is intriguing since previous studies have suggested that these nuclear receptors are important partners of the coactivator PGC-1 [symbol] key molecule in mitochondrial biogenesis (Puigserver et al. (2003). Endocr Rev 24, 78-90). While nuclear receptors are critical to mitochondrial biogenesis (Scarpulla, R. C. (2002). Biochim Biophys Acta 1576, 1-14), to our knowledge, none has previously been reported to be co-regulated with the mitochondrial genes themselves. Interestingly, a recent report demonstrated that PGC-1α co-activates Essra gene expression (Schreiber et al. (2003). J Biol Chem 278, 9013-8). Applicant's results raise the hypothesis that this may be a general phenomenon, in which PGC-1α is co-activating a number of its own transcriptional partners.

A number of other transcriptional regulators also have expression patterns very tightly regulated with the mitochondrial genes, including Mdfi, Nfix, Thx6, and Crsp2. These are excellent candidate transcription factors that may be targets of PGC-1α, or perhaps are involved in other mechanisms leading to the biogenesis of this organelle.

Surprisingly, the nutrient sensor Sir2 is also found within the mitochondrial expression neighborhood. Sir2 encodes an NAD(+)-dependent histone deacetylase which is homologous to the yeast silent information regulator 2 (ySir2). Sir2 is involved in gene silencing, chromosomal stability, and aging. Chromatin remodeling enzymes rely on coenzymes derived from metabolic pathways, including those generated by the mitochondrion. These observations suggest that Sir2 and mitochondrial gene expression are cooperatively regulated, perhaps linking the mitochondrion to the nutrient sensing activities of Sir2.

Third Experimental Series

The following experimental procedures were followed in the third experimental series:

Data Scaling, Visualization, and Annotation Enrichment. Microarray data were acquired and subjected to linear scaling using the median scan as a reference. Data were visualized using the dChip software package (10) and enrichment by ontology terms determined with the GoSurfer tool, using a P-value of 0.01 (11). Mitochondrial genes were defined based on a recent proteomic survey of organelle in mouse (12).

Promoter Databases. Applicants used the Reference Sequence annotations of mm3 build of the mouse genome (http://genome.ucsc.edu) and the annotation tables for the Affymetrix MG-U74Av2 chip (http://www.affymetrix.com) to compile a list of 5034 mouse genes for which there is a 1:1 mapping between Affymetrix probe-set and Reference Sequences. The ‘mouse promoter database’ consists of 2000 bp of genomic sequence centered on the annotated transcription start site of these genes.

Applicants also performed analyses on a ‘masked promoter database’, consisting of the regions within these 2000 bp that are aligned and conserved between mouse and human. Applicants used the mouse/human BLASTZ alignments (mouse mm3 vs. human hg15) (13) and only considered the 5008 promoters for which the alignment contained at least 100 bp. Applicants masked the aligned promoters to retain mouse sequence exhibiting at least 70% identity to human across windows of size 10. The median promoter length in the masked database is ˜1200 bp.

Motif discovery. For a given day, genes from the microarray are ordered on the basis of expression difference between GFP and PGC-1α (applicants use the signal to noise ratio as our difference metric). Each gene is annotated for the presence of a motif in the promoter by searching for exact k-mers (where k=6, 7, 8 or 9) or for selected motifs of interest. Applicants use the Mann-Whitney rank sum statistic U to determine whether the distribution of differential expression for those genes with a given motif differs from those genes lacking the motif. When working with promoters of unequal length (e.g., the masked promoter database), a more appropriate null hypothesis for the Mann-Whitney statistic is that the probability of detecting a motif in a promoter is proportional to its length. To assess the significance of a motif with rank sum U that appears in C promoters, applicants use Monte Carlo simulation (with 1000 samples) to estimate the null distribution of U for a sample of C ranks drawn randomly, without replacement, given relative weights proportional to the promoter lengths. For large C (C>10) and a reasonable distribution of promoter lengths, U is approximately normally distributed.

Promoter databases and motifADE source code are available at http://www-genome.wi.mit.edu/mpg/PGC_motifs/.

### Example 14

### Discovering Motifs Associated with Differential Expression

Systematic identification of transcription factors involved in biological processes in mammals remains a largely unsolved problem (17). A promising approach relates genome-wide expression profiles to promoter sequences to discover influential cis-motifs (18-21). Such methods have yielded impressive results in simple organisms such as yeast, but it has been challenging to extend these algorithms to mammalian genomes, where intergenic regions are large, annotation of gene structure is imperfect, and DNA sequence can be highly repetitive. Most of these methods seek motifs by comparison to a fixed background model of nucleotide composition (which fails to represent the fluctuations seen in large genomes) or by comparison between two sets of genes (which is likely to capture only very sharp differences). Further, many of these methods assume that the expression data are normally distributed, which may not always be true.

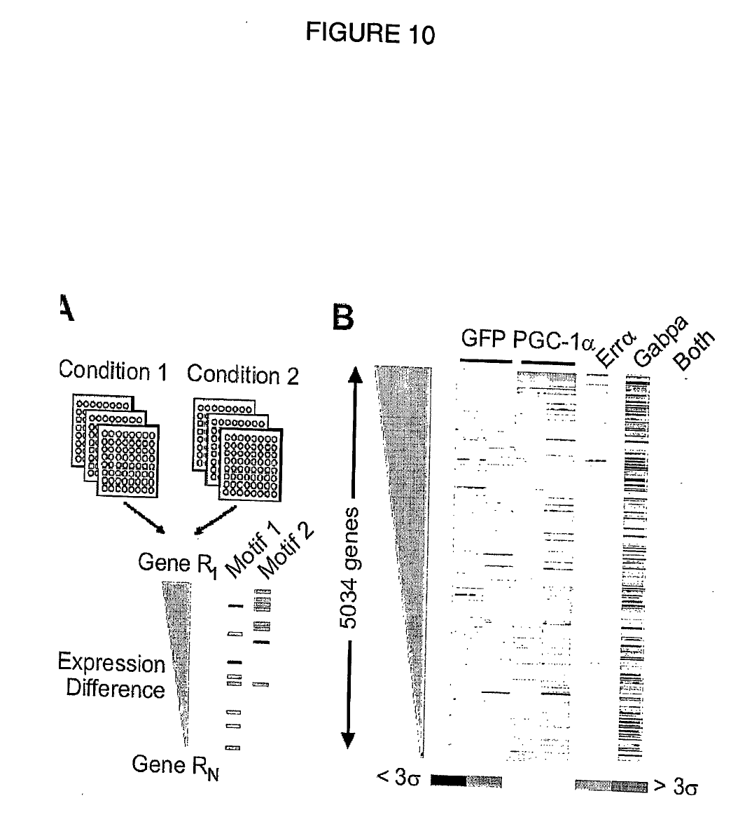

To overcome some of these obstacles, applicants devised a simple, nonparametric strategy for identifying motifs associated with differential expression (motifADE) (FIG. 10a). The algorithm involves three steps: (i) ranking genes based on differential expression between two conditions; (ii) given a candidate motif, identifying the subset of genes whose promoter regions contains the motif; and (iii) testing via a nonparametric, rank sum statistic (see Methods) if these genes tend to appear toward the top or bottom of the ranked list (indicating association) or are randomly distributed on the list. motifADE may be applied to a specific candidate motif of interest or to the list of all possible motifs of a given size (in which case the significance level should be adjusted to reflect multiple hypothesis testing). By using a nonparametric scoring procedure (see Methods), applicants do not make assumptions about the distribution of the expression data. Furthermore, by considering the entire rank ordered list, the promoters without the motif implicitly provide a background of DNA composition for comparison, and there is no need to group the genes into clusters. The method can operate on a traditional promoter database or even a database of promoters that have been masked based on evolutionary conservation (see Methods).

### Example 15

### Binding Sites for Errα and Gabpa are the Top Scoring Motifs Associated with the PGC-1α Transcriptional Program

To identify motifs related to PGC-1α action, applicants infected mouse C2C12 muscle cells with an adenovirus expressing PGC-1α and obtained gene expression profiles for 12,488 genes at 0, 1, 2, and 3 days following infection. Applicants found 649 genes that were induced at least 1.5-fold (nominal P<0.05) at day 3. As expected, these were enriched for genes involved in carbohydrate metabolism and the mitochondrion (see (1)). Interestingly, many genes involved with protein synthesis (GO terms: protein biosynthesis, mitochondrial ribosome and ribosome) are also induced.

Applicants then applied motifADE to study the 5034 mouse genes for which applicants have measures of gene expression as well as reliable annotations of the transcriptional start site (TSS) (see Methods). For each gene, the target region was defined to be a 21 kb region centered on the TSS. Applicants then tested all possible k-mers ranging in size from k=6 to k=9 nucleotides for association with differential expression on each of the three days of the timecourse. A total of 20 motifs achieved high statistical significance (p<0.001, following Bonferroni correction for multiple hypothesis testing) and these were almost exclusively related to two distinct motifs (see Table 8 and Table 9). The first motif, 5′-TGACCTTG-3′ was significant on days 1, 2, and 3 (adjusted P=2.1×10⁻⁶, 2.9×10⁻⁹, and 7.7×10⁻⁷, respectively). It corresponds to the published binding site for the orphan nuclear receptor Errα (22), which is known to be capable of being co-activated by PGC-1 [symbol] and -β (23-25). The Errα gene is known to be involved in metabolic processes, based on studies showing that knockout mice have reduced body weight and peripheral fat tissue, as well as altered expression of genes involved in metabolic pathways (26). The second motif is 5′-CTTCCG-3′ (adjusted p=8.9×10⁻⁹), which is the top scoring motif on day 3. It corresponds to the published binding site for Gabpa (27), which complexes with Gabpb (15) to form the heterodimer, nuclear respiratory factor-2 (NRF-2), a factor known to regulate the expression of some OXPHOS genes (28).

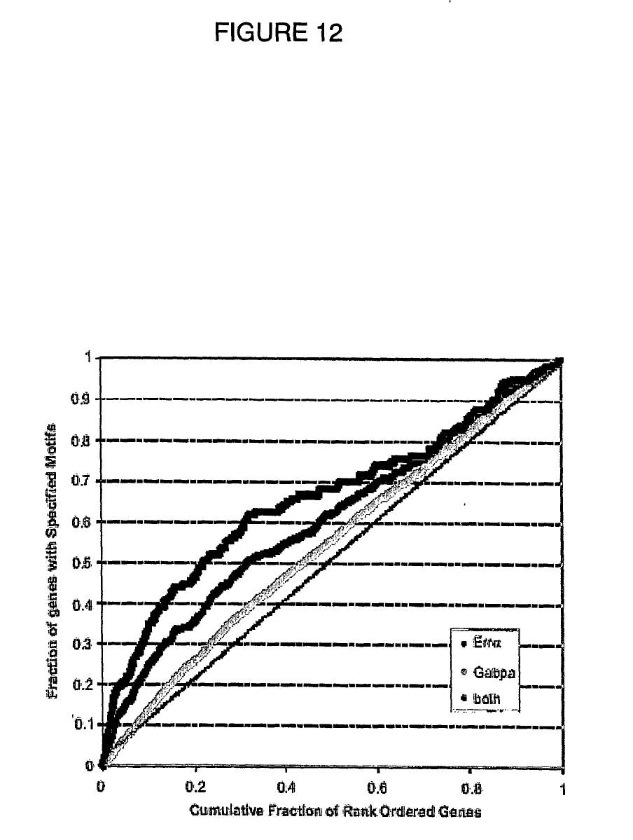

Interestingly, the reverse complements of these motifs did not score as well, suggesting a preference for the orientation of these motifs, and some occurrences of the motifs occurred downstream of the TSS. While each of these motifs is individually associated with PGC-1A, our analyses suggest that a gene having both motifs typically ranks higher on the list of differentially expressed genes and genes with only one of the motifs (FIG. 12) suggesting that the two motifs might have an additive or synergistic effect.

### Example 16

### Errα and Gabpa Motifs are Evolutionarily Conserved and Enriched Upstream of OXPHOS Genes

Applicants next repeated motifADE analysis using a “masked” promoter database (Table 3). Applicants still considered the 2000 bp centered on the TSS, but only considered those nucleotides aligned and conserved between mouse and human (see Methods). Still, the top ranking motifs on days 1 and 3 were related to Errα (day 1, P=4.8×10⁻⁶; day 3 P-1.2×10⁻¹¹) and to Gabpa (day 3 P=3.1×10⁻¹¹), providing additional support these motifs are biologically relevant.

The Errα and Gabpa motifs are particularly enriched upstream of the OXPHOS-CR genes, which exhibit reduced expression in human diabetes (5, 6). Whereas the top scoring Errα motif (5′-TGACCTTG-3′ or its reverse complement) only occurs in 12% of the promoters in the database, in 29% of the PGC-responsive genes (i.e., those genes induced at least 1.5 fold on day 3), and in 27% of the mitochondrial genes, they are found in 52% of the OXPHOS-CR genes (significance of enrichment, P-1×10⁻⁴). About one-half of these sites are perfectly conserved in the syntenic region in human. The top scoring Gabpa binding sites (5′-CTTCCG-3′ or its reverse complement) are much more common (62% of all promoters of the database and in 79% of the PGC-responsive genes), but they, too, show significant enrichment in the OXPHOS-CR genes (89%, P=0.02).

### Example 17

### Errα and Gabpa are Themselves Induced by PGC-1α

The above results suggest that Errα and Gabpa may be the key transcriptional factors mediating PGC-1α action in muscle. In this connection, it is notable that based on the microarray data, both Errα and Gabpa are themselves induced 2-fold (P<0.01) on day 1 following expression PGC-1 [symbol] consistent with previous studies (2, 23). Moreover, careful analysis of the Errα and Gabpa genes suggest that each contain potential binding sites for both transcription factors within the vicinity of their promoters. The Errα gene has the Errα motif as well as a conserved variant of the Gabpa binding site (27) upstream of the TSS, while the Gabpa gene has an Errα site upstream of the TSS and a conserved variant of the Gabpa binding site in its first intron These results raise the possibility that Errα and Gabpa may regulate their own and each other's expression.

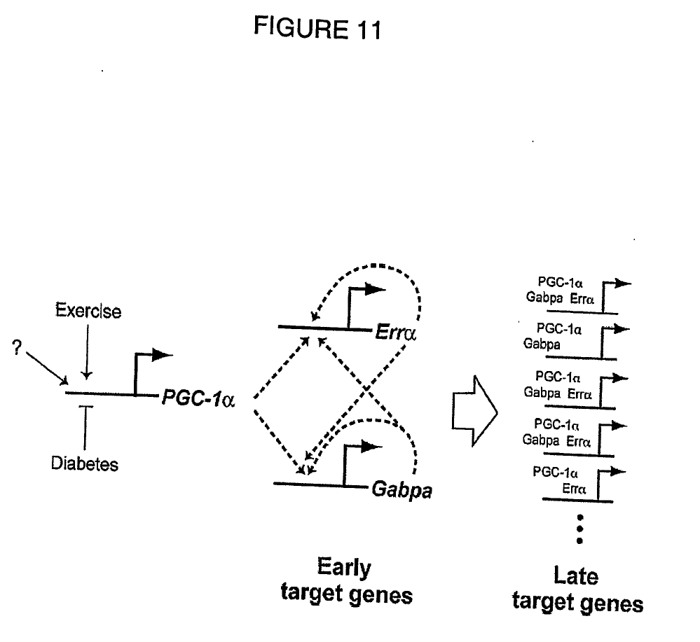

Taken together, the systematic analysis of the transcriptional program driven by PGC-1α in skeletal muscle suggests a model (FIG. 11) in which increases in PGC-1α protein levels (induced, for example, by exercise, e.g. see (29)) results in increased transcriptional activity of Gabpa and Errα on their own promoters, leading to a stable increase in the expression of these two factors via a double positive-feedback loop. These two factors, perhaps in combination with PGC-1α, are then crucial in the induction of downstream target genes, many of which have binding sites for these motifs (FIG. 11). Such a circuit may serve as a regulatory switch, analogous to a feed-forward loop that plays a key role in the early stages of endomesodermal development in sea urchin (30).

Experiment 18: MotifADE Results Applied to Human Diabetic Versus Normal Expression

Applicants applied the MotifADE method to analyze the transcription factor binding sites that are differentially expressed in diabetic vs. normal human skeletal muscle (previously published data, Mootha et al Nature Genetics 2003). The program identified exactly three motifs achieving an adjusted P-value<0.05. These are AAATCG (adjusted P-value 0.003), CCGGAAG (adjusted P-value 0.039), and AGCGTTT (adjusted P-value 0.011). Applicants note that the second motif is a published binding site for Gabpa (reverse complement of CTTCCG). This results suggest that Gabpa function is altered in diabetic muscle, or that perhaps another transcription factor that binds to this element.

Experiment 6: Identification of Human Genes Having Binding Sites for Errα, Gabpa or Both

Applicants searched for the binding sites motifs (forward or reverse complement) 3 Kb upstream and 1 Kb downstream of the annotated transcription start site. In the accompanying files are the genes with either one motif (forward or reverse complement) or both motifs conserved between human and mouse. The following genes were identified: Table 10: 678 genes with Errα motif conserved between mouse and human. Table 11: 2799 genes with Gabpa motif conserved between mouse and human. Table 12: 354 genes with both motifs conserved between mouse and human.

Discussion of First Experimental Series

In this study, applicants have used a combined genomic and computational strategy to systematically dissect a mammalian transcriptional circuit central to cellular energetics. The results above have computational, biological and medical implications.

First, the motifADE algorithm provides a simple, nonparametric approach for discovering cis-elements by considering differential gene expression. It makes very few assumptions about the statistical properties of DNA composition or about the distribution of gene expression. The method is flexible, and as applicants have shown, can easily incorporate “masked” or “phylogenetically footprinted” promoters. With additional cross-species comparisons, it should be possible to interrogate conserved segments of larger upstream regions (34). Moreover, the method operates on any ordered set of genes and is particularly convenient for discovering motifs associated with human disease states, e.g., “healthy versus sick” or “treated versus control.” Clearly, the method has some limitations. For example, in the current study, applicants were confident in the identity of the transcription factors binding the motifs discovered—in general this may not be the case, and experimental strategies will be needed to systematically determine the occupancy of newly identified motifs. Moreover, a motif may be missed if it lies outside the target promoter region, or if a functional binding site is too degenerate for our motif search strategy.

Second, the analyses above indicate that the immediate effects of PGC-1α on OXPHOS genes in muscle are largely mediated through Errα and Gabpa. Recent studies have shown that PGC-1β can also co-activate Errα (25). Together, the data imply a model of gene regulation in which PGC-1α (and likely PGC-1β) initially induces the expression of Errα and Gabpa, via a double positive feedback mechanism (FIG. 11). These transcription factors are then expressed at higher levels and are themselves co-activated by PGC-1 to induce downstream genes such as NRF-1 and members of OXPHOS. Certainly, other transcription factors and regulators, not identified in the current study, are involved in the mitochondrial biogenesis program. Whereas previous studies have shown that PGC-1 interacts with and/or induces 15-20 transcription factors in various physiological settings (including Errα and Gabpa (2, 23-25), the present study points to Errα and Gabpa as being especially important early in the timecourse in muscle and provides a model of how these factors interact in executing the transcriptional program.

Finally, the results suggest a potential approach to the treatment of type 2 diabetes. Recent studies in diabetic and pre-diabetic humans have demonstrated that there is a consistent decrease in the expression of genes of oxidative phosphorylation that are responsive to PGC-1α and PGC-1β and that treatments that induce PGC-1α (such as exercise) lead to increased expression of OXPHOS genes and improved insulin sensitivity (5, 6, 8, 9). On its face, this might argue for developing therapeutic approaches that raise the transcriptional activity of PGC-1. However, PGC-1 activates many different pathways in many tissues and such approaches may suffer from lack of specificity. For example, global transgenic overexpression of PGC-1β in mice results in resistance to obesity induced by a high-fat diet or by a genetic abnormality, though the contribution of PGC-1β expression in muscle has not been explored (25). On the other hand, a global knockout of Errα also causes a leaner phenotype and resistance to high-fat diet-induced obesity (26). The identification of the critical roles of Err [symbol] and Gabpa in mediating the transcriptional program altered in human diabetic muscle may offer a more specific target. Because Errα is an orphan nuclear receptor, it may be an attractive, “druggable” target for diabetes and for other human metabolic disorders.

### REFERENCES OF THIRD EXPERIMENTAL SECTION

1. Puigserver, P. & Spiegelman, B. M. (2003) Endocr Rev 24, 78-90.
2. Wu, Z., Puigserver, P., Andersson, U., Zhang, C., Adelmant, G., Mootha, V., Troy, A., Cinti, S., Lowell, B., Scarpulla, R. C., et al. (1999) Cell 98, 115-24.
3. Michael, L. F., Wu, Z., Cheatham, R. B., Puigserver, P., Adelmant, G., Lehman, J. J., Kelly, D. P. & Spiegelman, B. M. (2001) Proc Natl Acad Sci USA 98, 3820-5.
4. Lin, J., Wu, H., Tarr, P. T., Zhang, C. Y., Wu, Z., Boss, O., Michael, L. F., Puigserver, P., Isotani, B., Olson, E. N., et al. (2002) Nature 418, 797-801.
5. Patti, M. B., Butte, A. J., Crunkhorn, S., Cusi, K., Berria, R., Kashyap, S., Miyazaki, Y., Kohane, I., Costello, M., Saccone, R., et al. (2003) Proc Natl Acad Sci USA 100, 8466-71.
6. Mootha, V. K., Lindgren, C. M., Eriksson, K. F., Subramanian, A., Sihag, S., Lehar, J., Puigserver, P., Carlsson, E., Ridderstrale, M., Laurila, E., et al. (2003) Nat Genet 34, 267-73.
7. Kelley, D. E., He, J., Menshikova, E. V. & Ritov, V. B. (2002) Diabetes 51, 2944-50.
8. Petersen, K. F., Befroy, D., Dufour, S., Dziura, J., Ariyan, C., Rothman, D. L., DiPietro, L., Cline, G. W. & Shulman, G. I. (2003) Science 300, 1140-2.
9. Sreekumar, R., Halvatsiotis, P., Schimke, J. C. & Nair, K. S. (2002) Diabetes 51, 1913-20.
10. Schadt, E. E., Li, C., Ellis, B. & Wong, W. H. (2001) J Cell Biochem Suppl 37, 120-5.
11. Zhong, S., Li, C. & Wong, W. H. (2003) Nucleic Acids Res 31, 3483-6.
12. Mootha, V. K., Bunkenborg, J., Olsen, J. V., Hjerrild, M., Wisniewski, J. R., Stahl, E., Bolouri, M. S., Ray, H. N., Sihag, S., Kamal, M., et al. (2003) Cell 115, 629-40.
13. Schwartz, S., Kent, W. J., Smit, A., Zhang, Z., Baertsch, R., Hardison, R. C., Haussler, D. & Miller, W. (2003) Genome Res 13, 103-7.
14. Handschin, C., Rhee, J., Lin, J., Tarr, P. T. & Spiegelman, B. M. (2003) Proc Natl Acad Sci USA 100, 7111-6.
15. Batchelor, A. H., Piper, D. E., de la Brousse, F. C., McKnight, S. L. & Wolberger, C. (1998) Science 279, 1037-41.
16. St-Pierre, J., Lin, J., Krauss, S., Tarr, P. T., Yang, R., Newgard, C. B. & Spiegelman, B. M. (2003) J Biol Chem 278, 26597-603.
17. Qiu, P. (2003) Biochem Biophys Res Commun 309, 495-501.
18. Tavazoie, S., Hughes, J. D., Campbell, M. J., Cho, R. J. & Church, G. M. (1999) Nat Genet 22, 281-5.
19. Liu, X. S., Brutlag, D. L. & Liu, J. S. (2002) Nat Biotechnol 20, 835-9.
20. Conlon, E. M., Liu, X. S., Lieb, J. D. & Liu, J. S. (2003) Proc Natl Acad Sci USA 100, 3339-44.
21. Bussemaker, H. J., Li, H. & Siggia, E. D. (2001) Nat Genet 27, 167-71.
22. Johnston, S. D., Liu, X., Zuo, F., Eisenbraun, T. L., Wiley, S. R., Kraus, R. J. & Mertz, J. E. (1997) Mol Endocrinol 11, 342-52.
23. Schreiber, S. N., Knutti, D., Brogli, K., Uhlmann, T. & Kralli, A. (2003) J Biol Chem 278, 9013-8.
24. Huss, J. M., Kopp, R. P. & Kelly, D. P. (2002) J Biol Chem 277, 40265-74.
25. Kamei, Y., Ohizumi, H., Fujitani, Y., Nemoto, T., Tanaka, T., Takahashi, N., Kawada, T., Miyoshi, M., Ezaki, O. & Kakizuka, A. (2003) Proc Natl Acad Sci USA 100, 12378-83.
26. Luo, J., Sladek, R., Carrier, J., Bader, J. A., Richard, D. & Giguere, V. (2003) Mol Cell Biol 23, 7947-56.
27. Chinenov, Y., Coombs, C. & Martin, M. E. (2000) Gene 261, 311-20.
28. Virbasius, J. V. & Scarpulla, R. C. (1994) Proc Natl Acad Sci USA 91, 1309-13.
29. Russell, A. P., Feilchenfeldt, J., Schreiber, S., Praz, M., Crettenand, A., Gobelet, C., Meier, C. A., Bell, D. R., Kralli, A., Giacobino, J. P., et al. (2003) Diabetes 52, 2874-81.
30. Davidson, E. H., Rast, J. P., Oliveri, P., Ransick, A., Calestani, C., Yuh, C. H., Minokawa, T., Amore, G., Hinman, V., Arenas-Mena, C., et al. (2002) Science 295, 1669-78.
31. Sladek, R., Bader, J. A. & Giguere, V. (1997) Mol Cell Biol 17, 5400-9.
32. Scarpulla, R. C. (2002) Gene 286, 81-9.
33. Baar, K., Song, Z., Semenkovich, C. F., Jones, T. E., Han, D., H., Nolte, L. A., Ojuka, E. O., Chen, M. & Holloszy, J. O. (2003) FASEB J 17, 1666-73.
34. Kellis, M., Patterson, N., Endrizzi, M., Birren, B. & Lander, E. S. (2003) Nature 423, 241-54.
35. Stunnenberg, H. G. (1993) Bioessays 15, 309-15.
36. Chinenov, Y., Henzl, M. & Martin, M. B. (2000) J Biol Chem 275, 7749-56.

Tables:

TABLE 1
Clinical and biochemical characteristics of male subjects with normal glucose
tolerance (NGT), impaired glucose tolerance (IGT), and type 2 diabetes mellitus (DM2).

| | | Class | | P-Value | | |
|---|---|---|---|---|---|---|
| | NGT | IGT | DM2 | NGT vs. IGT | IGT vs. DM2 | NGT vs. DM2 |
| n | 17 | 8 | 18 | | | |
| Age (yrs) | 66.1 (1.0) | 66.4 (1.6) | 65.5 (1.8) | | | |
| BMI (kg/m²) | 23.6 (3.4) | 27.1 (4.8) | 27.3 (4.0) | | | 5.70 × 10⁻³ |
| WHR | 0.91 (0.09) | 0.97 (0.04) | 0.99 (0.03) | 3.00 × 10⁻² | | 3.83 × 10⁻³ |
| Trigs (mmol/L) | 1.03 (0.40) | 1.83 (1.60) | 2.04 (1.13) | | | 2.63 × 10⁻³ |
| Chol (mmol/L) | 5.39 (0.09) | 4.60 (1.48) | 5.77 (0.97) | | | |
| OGTT | | | | | | |
| Glucose 0 (mmol/L) | 4.67 (0.50) | 5.05 (0.46) | 7.83 (2.3) | | 9.22 × 10⁻⁵ | 2.01 × 10⁻⁵ |
| Insulin 0 (uU/ml) | 5.41 (3.3) | 13.38 (8.9) | 12.0 (6.0) | 4.05 × 10⁻² | | 4.10 × 10⁻⁴ |
| Glucose 120 (mmol/L) | 6.58 (0.94) | 9.15 (0.8) | 14.9 (4.0) | 2.51 × 10⁻⁶ | 8.91 × 10⁻⁶ | 4.90 × 10⁻⁸ |
| Insulin 120 (uU/ml) | 33.5 (19.3) | 125.1 (66.1) | 43.5 (25.6) | 5.47 × 10⁻³ | 9.73 × 10⁻³ | |
| M-value (mg/kg/min) | 8.74 (3.15) | 6.32 (3.08) | 4.22 (1.72) | | | 2.30 × 10⁻⁵ |
| VO2max (ml O₂/kg/min) | 32.1 (5.46) | 26.5 (4.6) | 24.3 (5.6) | 1.72 × 10⁻² | | 3.09 × 10⁻⁴ |
| Glycogen (mmol/kg) | 371.1 (77.0) | 326.5 (88.0) | 350.6 (97.8) | | | |
| Type I Fibers | | | | | | |
| Number (%) | 37.2 (13.5) | 33.5 (3.6) | 36.4 (9.3) | | | |
| Area (%) | 39.1 (14.4) | 32.7 (0.91) | 40.1 (10.7) | | 2.35 × 10⁻² | |
| Capillaries/Fiber | 3.91 (0.72) | 4.05 (1.04) | 4.14 (0.75) | | | |
| Type IIb Fibers | | | | | | |
| Number (%) | 73.8 (42.1) | 60.2 (51.4) | 72.2 (36.7) | | | |
| Area (%) | 31.3 (18.0) | 24.7 (18.3) | 36.2 (15.4) | | | |
| Capillaries/Fiber | 2.97 (0.71) | 3.05 (0.87) | 3.02 (0.65) | | | |

Values are mean (S.D.).
M-value is the total body glucose uptake.
VO2max is the total body aerobic capacity.
Only P-values < 0.05 are shown for pairwise comparisons, using a two-sided t-test.

TABLE 2
149 gene sets considered in the current analysis.

Pathways Curated at WICGR
FFA Oxidation
Gluconeogenesis
Glycolysis
Glycogen metabolism
GO: 0005739
Insulin signaling
Ketone body metabolism
Pyruvate metabolism
Reactive oxygen species
Kreb's cycle
Oxidative phosphorylation (OXPHOS)
human_mitoDB_6_2002
mitochondria keyword
36 GNF Mouse Expression
Clusters
cluster c0, . . . , cluster c35
Pathways from NetAFFX (October 2002)
MAP00010_Glycolysis_Gluconeogenesis
MAP00020_Citrate_cycle_TCA_cycle
MAP00030_Pentose_phosphate_pathway
MAP00031_Inositol_metabolism
MAP00040_Pentose_and_glucuronate_interconversions
MAP00051_Fructose_and_mannose_metabolism
MAP00052_Galactose_metabolism
MAP00053_Ascorbate_and_aldarate_metabolism
MAP00061_Fatty_acid_biosynthesis_path_1
MAP00062_Fatty_acid_biosynthesis_path_2
MAP00071_Fatty_acid_metabolism
MAP00072_Synthesis_and_degradation_of_ketone_bodies
MAP00100_Sterol_biosynthesis
MAP00120_Bile_acid_biosynthesis
MAP00130_Ubiquinone_biosynthesis
MAP00140_C21_Steroid_hormone_metabolism
MAP00150_Androgen_and_estrogen_metabolism
MAP00190_Oxidative_phosphorylation
MAP00193_ATP_synthesis
MAP00195_Photosynthesis
MAP00220_Urea_cycle_and_metabolism_of_amino_groups
MAP00230_Purine_metabolism
MAP00240_Pyrimidine_metabolism
MAP00251_Glutamate_metabolism
MAP00252_Alanine_and_aspartate_metabolism
MAP00253_Tetracycline_biosynthesis
MAP00260_Glycine_serine_and_threonine_metabolism
MAP00271_Methionine_metabolism
MAP00272_Cysteine_metabolism
MAP00280_Valine_leucine_and_isoleucine_degradation
MAP00290_Valine_leucine_and_isoleucine_biosynthesis
MAP00300_Lysine_biosynthesis
MAP00310_Lysine_degradation
MAP00330_Arginine_and_proline_metabolism
MAP00340_Histidine_metabolism
MAP00350_Tyrosine_metabolism
MAP00360_Phenylalanine_metabolism
MAP00361_gamma_Hexachlorocyclohexane_degradation
MAP00380_Tryptophan_metabolism
MAP00400_Phenylalanine_tyrosine_and_tryptophan_biosynthesis
MAP00410_beta_Alanine_metabolism
MAP00430_Taurine_and_hypotaurine_metabolism
MAP00440_Aminophosphonate_metabolism
MAP00450_Selenoamino_acid_metabolism
MAP00460_Cyanoamino_acid_metabolism
MAP00471_D_Glutamine_and_D_glutamate_metabolism
MAP00472_D_Arginine_and_D_ornithine_metabolism
MAP00480_Glutathione_metabolism
MAP00500_Starch_and_sucrose_metabolism
MAP00510_N_Glycans_biosynthesis
MAP00511_N_Glycan_degradation
MAP00512_O_Glycans_biosynthesis
MAP00520_Nucleotide_sugars_metabolism
MAP00521_Streptomycin_biosynthesis
MAP00522_Erythromycin_biosynthesis
MAP00530_Aminosugars_metabolism
MAP00531_Glycosaminoglycan_degradation
MAP00532_Chondroitin_Heparan_sulfate_biosynthesis
MAP00533_Keratan_sulfate_biosynthesis
MAP00550_Peptidoglycan_biosynthesis
MAP00561_Glycerolipid_metabolism
MAP00562_Inositol_phosphate_metabolism
MAP00570_Sphingophospholipid_biosynthesis
MAP00580_Phospholipid_degradation
MAP00590_Prostaglandin_and_leukotriene_metabolism
MAP00600_Sphingoglycolipid_metabolism
MAP00601_Blood_group_glycolipid_biosynthesis_lact_series
MAP00602_Blood_group_glycolipid_biosynthesis_neolact_series
MAP00603_Globoside_metabolism
MAP00620_Pyruvate_metabolism
MAP00625_Tetrachloroethene_degradation
MAP00630_Glyoxylate_and_dicarboxylate_metabolism
MAP00631_1_2_Dichloroethane_degradation
MAP00632_Benzoate_degradation
MAP00640_Propanoate_metabolism
MAP00643_Styrene_degradation
MAP00650_Butanoate_metabolism
MAP00670_One_carbon_pool_by_folate
MAP00680_Methane_metabolism
MAP00710_Carbon_fixation
MAP00720_Reductive_carboxylate_cycle_CO2_fixation
MAP00740_Riboflavin_metabolism
MAP00750_Vitamin_B6_metabolism
MAP00760_Nicotinate_and_nicotinamide_metabolism
MAP00770_Pantothenate_and_CoA_biosynthesis
MAP00780_Biotin_metabolism
MAP00790_Folate_biosynthesis
MAP00830_Retinol_metabolism
MAP00860_Porphyrin_and_chlorophyll_metabolism
MAP00900_Terpenoid_biosynthesis
MAP00910_Nitrogen_metabolism
MAP00920_Sulfur_metabolism
MAP00940_Flavonoids_stilbene_and_lignin_biosynthesis
MAP00950_Alkaloid_biosynthesis_I
MAP00960_Alkaloid_biosynthesis_II
MAP00970_Aminoacyl_tRNA_biosynthesis
MAP03020_RNA_polymerase
MAP03030_DNA_polymerase
MAP03070_Type_III_secretion_system
MAP03090_Type_II_secretion_system

TABLE 3
Genes in the mitochondria expression neighborhood with putative roles
in DNA maintenance and repair based on Gene Ontology annotations.
The gene name, symbol, and neighborhood index (N₁₀₀) are
provided for each gene.

| | | |
|---|---|---|
| | Gene | |
| Gene name | symbol | N₁₀₀ |

Transcriptional regulators

| | | |
|---|---|---|
| MyoD family inhibitor | Mdfi | 63 |
| nuclear factor I/X | Nfix | 60 |
| zinc finger protein 288 | Zfp288 | 56 |
| T-box 6 | Tbx6 | 49 |
| Cofactor required for Sp1 transcriptional activation subunit 2 | Crsp2 | 47 |
| RIKEN cDNA 9130025P16 gene | 9130025P16Rik | 46 |
| Kruppel-like factor 9 | Klf9 | 43 |
| EGL nine homolog 1 | Egln1 | 39 |
| Estrogen related receptor, alpha | Esrra | 36 |
| nuclease sensitive element binding protein 1 | Nsep1 | 34 |
| sirtuin 1 (silent mating type information regulation 2, | Sirt1 | 31 |
| homolog) | | |
| peroxisome proliferator activated receptor alpha | Ppara | 29 |
| metastasis associated 1-like 1 | Mta1l1 | 28 |
| NK2 transcription factor related, locus 5 | Nkx2-5 | 27 |
| cardiac responsive adriamycin protein | Crap | 24 |
| homeo box D8 | Hoxd8 | 21 |
| nuclear receptor subfamily 1, group I, member 2 | Nr1i2 | 21 |
| nuclear receptor subfamily 1, group H, member 3 | Nr1h3 | 20 |
| cellular nucleic acid binding protein | Cnbp | 19 |
| transcription factor 2 | Tcf2 | 19 |
| Est2 repressor factor | Erf | 19 |
| nuclear receptor subfamily 5, group A, member 1 | Nr5a1 | 18 |
| nuclear factor, erythroid derived 2, -like 1 | Nfe2l1 | 18 |
| zinc finger protein 30 | Zfp30 | 17 |
| peroxisome proliferator activated receptor gamma | Pparg | 17 |
| cAMP responsive element binding protein 1 | Creb1 | 15 |
| SRY-box containing gene 6 | Sox6 | 15 |
| CCAAT/enhancer binding protein (C/EBP), alpha | Cebpa | 15 |

DNA repair

| | | |
|---|---|---|
| mutL homolog 1 | Mlh1 | 29 |
| mutS homolog 5 | Msh5 | 24 |
| excision repair cross-complementing rodent repair | Ercc1 | 15 |
| deficiency, complementation group 1 | | |

TABLE 4
Annotation and experimental support for the mito-A proteins. The mito-A list of protein clusters consist of proteins that
are physically associated with mitochondria, based on previous annotations or based on organelle proteomics. The
list is produced by pooling all the individual proteins identified in the organelle proteomics survey with proteins
previously annotated as being mitochondrial. These proteins were then clustered into 601 groups using a BLAST
procedure (see Methods). Each cluster may be supported by previous annotations, organelle proteomics, or by
both (protein accessions are indicated in the appropriate columns). Of the 601 clusters, 10 correspond to expected
contaminants and have been flagged. The remaining 591 constitute the mito-A list that is used in the analysis.
For each mito-A cluster, an exemplar protein (typically corresponding to a Reference Sequence) accession
and description are provided. GenPept or Swissprot accession numbers of the cluster members are provided
in the appropriate columns. Of the 591 mito-A clusters, 37 appeared to be obviously mitochondrial based on the
description, so these have been flagged as mitochondrial in a dedicated column called “by name.”

| | | Previous Mitochondral Annotations | | | |
|---|---|---|---|---|---|
| Exemplar Protein for the Cluster | | LocusLink | | LocusLink | |
| Accession | Description | Mouse | MITOP Mouse | Human | MITOP Human |
| 19354491 | 1110020P15Rik protein [Mus musculus] | | | | |
| 13385680 | 2,4-dienoyl CoA reductase 1, mitochondrial [Mus | | | 4503301 | S53352 |
| | musculus] | | | | |
| 20071710 | 2010002H18Rik protein [Mus musculus] | | | | |
| 21630283 | 2′-5′ oligoadenylate synthetase 1A [Mus musculus] | | P29080 P11928 | | P1_A22842 |
| | | | | | B24359 |
| | | | | | A91013 |
| 21644597 | 2′-5′ oligoadenylate synthetase 2; 2′-5′ oligoadenylate | | | | B42665 A42665 |
| | synthetase-like 11 [Mus | | | | |
| 6680233 | 3-hydroxy-3-methylglutaryl-Coenzyme A lyase [Mus | 25022682 | HMGL_MOUSE | | A45470 |
| | musculus] | 25049209 | | | |
| | | 6680233 | | | |
| 31560689 | 3-hydroxy-3-methylglutaryl-Coenzyme A synthase 2 | 27734729 | B55729 | 5031751 | S51103 |
| | [Mus musculus] | 20965433 | | | |
| | | 20874930 | | | |
| 31982169 | 3-hydroxybutyrate dehydrogenase (heart, | | | 17738292 | A42845 |
| | mitochondrial); 3-hydroxybutyrate | | | | |
| 21704140 | 3-hydroxyisobutyrate dehydrogenase, mitochondrial | | | | D3HI_HUMAN |
| | precursor; EST AI265272; | | | | |
| 20149758 | 3-mercaptopyruvate sulfurtransferase; e [Mus | | | | ROHU |
| | musculus] | | | | |
| 481864 | 3-methyl-2-oxobutanoate dehydrogenase (lipoamide) | | S39807 | 4557353 | A37157 |
| | (EC 1.2.4.4) - mouse | | | | |
| 18266680 | 3-oxoacid CoA transferase [Mus musculus] | | | | SCOT_HUMAN |
| 11968160 | 3-oxoacid CoA transferase 2A; haploid germ cell | | | | |
| | specific succinyl CoA | | | | |
| 6679066 | 4-nitrophenylphosphatase domain and non-neuronal | | | | |
| | SNAP25-like protein homolog 1 | | | | |
| 20127399 | 5′,3′-deoxyribonucleotidase, mitochondrial [Mus | 20127399 | | 9910372 | |
| | musculus] | | | | |
| 18921208 | 8-oxoguanine DNA-glycosylase 1 [Mus musculus] | 18921208 | | | |
| 9910174 | A kinase (PRKA) anchor protein 10; protein kinase A | 9910174 | | | |
| | anchoring protein [Mus | | | | |
| 30725845 | AAA-ATPase TOB3 [Mus musculus] | | | | |
| 1167982 | ABC transporter-7 | | | | ABC7_HUMAN |
| 21450129 | acetyl-Coenzyme A acetyltransferase 1 precursor [Mus | 21450129 | | 4557237 | JH0255 |
| | musculus] | | | | |
| 29126205 | acetyl-Coenzyme A acyltransferase 2 (mitochondrial 3- | | | 5174429 | S43440 |
| | oxoacyl-Coenzyme A | | | | |
| 20841184 | acetyl-Coenzyme A carboxylase beta [Mus musculus] | | | | |
| 31982520 | acetyl-Coenzyme A dehydrogenase, long-chain [Mus | 6680616 | ACDL_MOUSE | 4501857 | A40559 |
| | musculus] | 25020672 | | | |
| 6680618 | acetyl-Coenzyme A dehydrogenase, medium chain | 6680618 | A55724 | 4557231 | I52240 |
| | [Mus musculus] | | | | |
| 9790059 | acid phosphatase 6, lysophosphatidic; acid | | | 21359911 | |
| | phosphatase like 1 [Mus musculus] | | | | |
| 18079339 | aconitase 2, mitochondrial [Mus musculus] | 18079339 | | 4501867 | Q99798 |
| 8850209 | actin-like [Mus musculus] | | | | |
| 31982522 | acyl-Coenzyme A dehydrogenase, short chain; acetyl- | 6680620 | I49605 | 4557233 | A30605 |
| | Coenzyme A dehydrogenase, | | | | |
| 17647119 | acyl-Coenzyme A dehydrogenase, short/branched | | | 4501859 | A55680 |
| | chain [Mus musculus] | | | | |
| 23956084 | acyl-Coenzyme A dehydrogenase, very long chain [Mus | 23956084 | ACDV_MOUSE | 4557235 | ACDB_HUMAN |
| | musculus] | 25056160 | | | |
| | | 20881925 | | | |
| 7656855 | acyl-Coenzyme A oxidase 1, palmitoyl; acyl-Coenzyme | | | | |
| | A oxidase; Acyl-CoA oxidase | | | | |
| 12331400 | acyl-Coenzyme A thioesterase 3, mitochondrial; MT- | 12331400 | | 6912518 | |
| | ACT48, p48 [Mus musculus] | 9790025 | | | |
| 6753074 | adaptor protein complex AP-2, mu1; adaptor-related | | | | |
| | protein complex AP-2, mu1; | | | | |
| 10946936 | adenylate kinase 1; cytosolic adenylate kinase [Mus | | | | |
| | musculus] | | | | |
| 34328230 | adenylate kinase 2 [Mus musculus] | 8392883 | | | KAD2_HUMAN |
| 23956104 | adenylate kinase 3 alpha-like; adenylate kinase 3 alpha | | | | |
| | like [Mus musculus] | | | | |
| 6753022 | adenylate kinase 4 [Mus musculus] | 6753022 | | | KIHUA3 |
| 16905099 | AFG3(ATPase family gene 3)-like 1 [Mus musculus] | 16905099 | | 5802970 | |
| 6753030 | A-kinase anchor protein 1; A kinase anchor protein | 6753030 | | | I39173 |
| | [Mus musculus] | | | | |
| 7709978 | alanine-glyoxylate aminotransferase; alanine-glyoxylate | 7709978 | | | P21549 XNHUSP |
| | aminotransferase 1 [Mus | | | | |
| 6753036 | aldehyde dehydrogenase 2, mitochondrial [Mus | 6753036 | I48966 | 25777732 | A40872 DEHUE2 |
| | musculus] | | | | |
| 19527258 | aldehyde dehydrogenase family 6, subfamily A1 [Mus | | | | MMSA_HUMAN |
| | musculus] | | | | |
| 20070418 | aldehyde dehydrogenase family 7, member A1; | | | | |
| | aldehyde dehydrogenase 7 family, | | | | |
| 27659728 | aldo-keto reductase family 7, member A5 (aflatoxin | | | | |
| | aldehyde reductase); | | | | |
| 13435924 | aldolase 3, C isoform [Mus musculus] | | | | |
| 6678766 | alpha-methylacyl-CoA racemase; alpha-methylacyl- | 6678766 | | 23618869 | |
| | Coenzyme A racemase; | | | | |
| 31980703 | aminoadipate-semialdehyde synthase; lysine | | | | |
| | oxoglutarate reductase, saccharopine | | | | |
| 33859502 | aminolevulinic acid synthase 2, erythroid; erythroid- | 20985872 | SYMSAL | | SYHUAL SYHUAE |
| | specific ALAS; | | | | |
| 13507620 | ankycorbin; NORPEG-like protein [Mus musculus] | | | | |
| 6753058 | annexin A10 [Mus musculus] | | | | |
| 21541818 | AP endonuclease 2 [Mus musculus] | 21541818 | | | |
| 6753110 | arginase type II [Mus musculus] | 6753110 | | 4502215 | ARG2_HUMAN |
| 25089776 | ATP synthase D chain, mitochondrial | | PN0046 | | |
| 5834959 | ATP synthase F0 subunit 6 [Mus musculus] | 5834959 | PWMS6 | 27754208 | PWHU6 |
| 5834958 | ATP synthase F0 subunit 8 [Mus musculus] | 5834958 | PWMS8 | | PWHU8 |
| 21263432 | ATP synthase gamma chain, mitochondrial precursor | | PT0095 | | |
| 31980648 | ATP synthase, H+ transporting mitochondrial F1 | 25052136 | P56480 | 4502295 | A33370 |
| | complex, beta subunit; ATP | 7949003 | | | |
| 33859512 | ATP synthase, H+ transporting, mitochondrial F0 | 20875157 | | 21361565 | JQ1144 |
| | complex, subunit b, isoform 1 | 25020502 | | | |
| 31982497 | ATP synthase, H+ transporting, mitochondrial F0 | 6680750 | AT91_MOUSE | | I38612 S34067 |
| | complex, subunit c (subunit 9), | | | | S34066 |
| 10181184 | ATP synthase, H+ transporting, mitochondrial F0 | 10181184 | P56135 | | |
| | complex, subunit f, isoform 2; | | | | |
| 7949005 | ATP synthase, H+ transporting, mitochondrial F0 | 7949005 | PD0444 | 18644883 | JT0563 |
| | complex, subunit F; | | | | |
| 31980744 | ATP synthase, H+ transporting, mitochondrial F0 | | | | |
| | complex, subunit g; F1F0-ATP | | | | |
| 6680748 | ATP synthase, H+ transporting, mitochondrial F1 | 6680748 | JC1473 | 4757810 | PWHUA |
| | complex, alpha subunit, isoform | | | | |
| 13385484 | ATP synthase, H+ transporting, mitochondrial F1 | | | 5901896 | |
| | complex, epsilon subunit; ATP | | | | |
| 11602916 | ATP synthase, H+ transporting, mitochondrial F1 | 11602916 | | 4885079 | A49108 |
| | complex, gamma polypeptide 1; F1 | | | | |
| 20070412 | ATP synthase, H+ transporting, mitochondrial F1 | | | | ATPO_HUMAN |
| | complex, O subunit [Mus | | | | |
| 6671592 | ATP synthase, H+ transporting, mitochondrial F1F0 | 6671592 | JC1412 | | |
| | complex, subunit e [Mus | | | | |
| 31982864 | ATPase inhibitor [Mus musculus] | 6671594 | | 7705927 | JC7175 |
| 6680758 | ATPase, Cu++ transporting, beta polypeptide; Wilson | | | | S40525 |
| | protein; toxic milk [Mus | | | | |
| 31560731 | ATPase, H+ transporting, V1 subunit A, isoform 1; | | | | |
| | ATPase, H+ transporting, | | | | |
| 6680756 | ATPase, H+ transporting, V1 subunit E isoform 1; | | | | |
| | ATPase, H+ transporting | | | | |
| 6680612 | ATP-binding cassette, sub-family D, member 3; | | | | |
| | peroxisomal membrane protein, 70 | | | | |
| 3766201 | ATP-specific succinyl-CoA synthetase beta subunit | 20876884 | | | |
| | [Mus musculus] | | | | |
| 7709988 | AU RNA-binding enoyl-coenzyme A hydratase; AU | 7709988 | | 18426971 | |
| | RNA-binding protein/enoyl-coenzyme | 25052987 | | | |
| 6753168 | B-cell leukemia/lymphoma 2 [Mus musculus] | 6753168 | TVMSA1 B25960 | | TVHUA1 D37332 |
| 6671622 | B-cell receptor-associated protein 37; repressor of | | | | |
| | estrogen receptor activity | | | | |
| 6753198 | BCL2/adenovirus E1B 19 kDa-interacting protein 1, | 6753198 | | | |
| | NIP3; BCL2/adenovirus E1B 19 | | | | |
| 6753200 | BCL2/adenovirus E1B 19 kDa-interacting protein 3-like; | 6753200 | | 4757860 | NIPL_HUMAN |
| | BCL2/adenovirus E1B 19 | | | | |
| 6680770 | Bcl2-associated X protein [Mus musculus] | | BAXA_MOUSE | | BAXA_HUMAN |
| 31981887 | Bcl2-like [Mus musculus] | 6753170 | | 20336335 | BCLX_HUMAN |
| | | | | 4502381 | |
| 31981875 | benzodiazepine receptor, peripheral [Mus musculus] | 6753216 | A53405 | | I38105 |
| 31542228 | BH3 interacting domain death agonist [Mus musculus] | | | 4557361 | BID_HUMAN |
| 9055178 | brain protein 44-like; apoptosis-regulating basic protein | | | | |
| | [Mus musculus] | | | | |
| 33859514 | branched chain aminotransferase 2, mitochondrial [Mus | 23597235 | | 4502375 | BCAM_HUMAN |
| | musculus] | | | | |
| 31982494 | branched chain ketoacid dehydrogenase E1, alpha | 6671624 | S71881 | 11386135 | DEHUXA |
| | polypeptide; BCKAD E1[a] [Mus | | | | |
| 6753164 | branched chain ketoacid dehydrogenase kinase; | 6753164 | | 5031609 | |
| | branched chain keto acid | | | | |
| 16905127 | butyryl Coenzyme A synthetase 1; acetyl-Coenzyme A | | | | |
| | synthetase 3 [Mus musculus] | | | | |
| 6753290 | calsequestrin 1 [Mus musculus] | | | | A60424 |
| 7381085 | carbamoylphosphate synthetase I [Mus musculus] | | | 21361331 | JQ1348 |
| 6671680 | carbonic anhydrase 5a, mitochondrial; carbonic | 6671680 | S12579 | 4502521 | CRHU5 |
| | anhydrase 5, mitochondrial; | | | | |
| 9506463 | carbonic anhydrase 5b, mitochondrial; carbonic | 9506463 | | 6005723 | |
| | anhydrase VB; carbonic anhydrase | | | | |
| 6671688 | carbonyl reductase 2; lung carbonyl reductase [Mus | 6671688 | A28053 | | |
| | musculus] | | | | |
| 6681009 | carnitine acetyltransferase [Mus musculus] | 6681009 | CACP_MOUSE | 21618331 | A55720 |
| | | | | 21618334 | |
| | | | | 21618336 | |
| 27804309 | carnitine palmitoyltransferase 1, liver; L-CPT I [Mus | 20884997 | | 4503021 | I59351 |
| | musculus] | 27804309 | | | |
| 6753512 | carnitine palmitoyltransferase 1, muscle; M-CPT I [Mus | | | 23238254 | S70579 |
| | musculus] | | | 23238256 | |
| | | | | 4758050 | |
| | | | | 23238258 | |
| 6753514 | carnitine palmitoyltransferase 2; CPT II [Mus musculus] | 6753514 | A49362 | | A39018 |
| 6753454 | caseinolytic protease X [Mus musculus] | 6753454 | | 7242140 | CLPX_HUMAN |
| 8393156 | caseinolytic protease, ATP-dependent, proteolytic | 8393156 | | | S68421 |
| | subunit homolog; caseinolytic | | | | |
| 20847456 | caspase 8 [Mus musculus] | | | 15718704 | |
| | | | | 15718706 | |
| | | | | 15718708 | |
| | | | | 15718710 | |
| | | | | 15718712 | |
| 6753272 | catalase; catalase 1 [Mus musculus] | | | | |
| 6681079 | cathepsin B preproprotein [Mus musculus] | | | | |
| 6753556 | cathepsin D [Mus musculus] | | | | |
| 11968166 | cathepsin Z preproprotein; cathepsin Z precursor; | | | | |
| | cathepsin X [Mus musculus] | | | | |
| 31560609 | ceroid lipofuscinosis, neuronal 3, juvenile (Batten, | | | 4502889 | |
| | Spielmeyer-Vogt disease) | | | | |
| 6753448 | ceroid-lipofuscinosis, neuronal 2 [Mus musculus] | | | | |
| 7304963 | chloride intracellular channel 4 (mitochondrial) [Mus | 7304963 | | | |
| | musculus] | | | | |
| 13385942 | citrate synthase [Mus musculus] | | | 4758076 | |
| 6680816 | complement component 1, q subcomponent binding | 6680816 | | | |
| | protein [Mus musculus] | | | | |
| 6681007 | coproporphyrinogen oxidase; clone 560 [Mus musculus] | 6681007 | A48049 | 20127406 | I52444 |
| 10946574 | creatine kinase, brain [Mus musculus] | | | | |
| 6753428 | creatine kinase, mitochondrial 1, ubiquitous [Mus | 6753428 | S24612 | 4502855 | A35756 A30789 |
| | musculus] | | | 10334859 | |
| 6681031 | cryptochrome 1 (photolyase-like) [Mus musculus] | | | 4758072 | |
| 201006 | Cu/Zn-superoxide dismutase | | | | |
| 5834966 | cytochrome b [Mus musculus] | 5834966 | CBMS | | CBHU |
| 22094077 | cytochrome b-245, alpha polypeptide; cytochrome beta- | | | 4557505 | |
| | 558; p22 phox [Mus | | | | |
| 31542440 | cytochrome b-245, beta polypeptide [Mus musculus] | | | 6996021 | |
| 13385268 | cytochrome b-5 [Mus musculus] | | | 4503183 | CBHU5 CBHU5E |
| 5834956 | cytochrome c oxidase subunit I [Mus musculus] | 5834956 | ODMS1 | 27754204 | ODHU1 |
| 5834957 | cytochrome c oxidase subunit II [Mus musculus] | 5834957 | OBMS2 | 27754206 | OBHU2 |
| 5834960 | cytochrome c oxidase subunit III [Mus musculus] | 5834960 | OTMS3 | | OTHU3 |
| 16716379 | cytochrome c oxidase subunit IV isoform 2 precursor; | 16716379 | | | |
| | Cox IV-2 [Mus musculus] | | | | |
| 6677977 | cytochrome c oxidase subunit VIIa polypeptide 2-like; | 6677977 | | | O14548 |
| | silica-induced gene 81 | | | | |
| 13384754 | cytochrome c oxidase subunit VIIb [Mus musculus] | 13384754 | | 4502991 | OSHU7B |
| 6753498 | cytochrome c oxidase, subunit IVa; cytochrome c | 6753498 | S12142 | | OLHU4 |
| | oxidase, subunit IV [Mus | | | | |
| 6680986 | cytochrome c oxidase, subunit Va [Mus musculus] | 6680986 | S05495 | 4758038 | OTHU5A |
| 6753500 | cytochrome c oxidase, subunit Vb [Mus musculus] | 6753500 | A39425 | | OTHU5B |
| 6680988 | cytochrome c oxidase, subunit VI a, polypeptide 1; | 6680988 | S52088 | | OGHU6L |
| | subunit VIaL (liver-type) | | | | |
| 6753502 | cytochrome c oxidase, subunit VI a, polypeptide 2; | 6753502 | COXD_MOUSE | | OGHU6A |
| | subunit VIaH (heart-type) | | | | |
| 13385090 | cytochrome c oxidase, subunit VIb [Mus musculus] | | | | OGHU6B |
| 16716343 | cytochrome c oxidase, subunit VIc [Mus musculus] | | S16083 | | OGHU6C |
| 6753504 | cytochrome c oxidase, subunit VIIa 1; cytochrome c | 6753504 | | | |
| | oxidase subunit VIIa 1 [Mus | | | | |
| 31981830 | cytochrome c oxidase, subunit VIIa 2; cytochrome c | 6753506 | I48286 | | |
| | oxidase subunit VIIa 3; | | | | |
| 6680991 | cytochrome c oxidase, subunit VIIc; cytochrome c | 25025041 | COXO_MOUSE | | OSHU7C |
| | oxidase subunit VIIc [Mus | 25053109 | S10303 | | |
| | | 25057077 | | | |
| | | 6680991 | | | |
| 6680993 | cytochrome c oxidase, subunit VIIIa; COX VIII-L [Mus | 6680993 | COXR_MOUSE | 4758044 | OSHU8 |
| | musculus] | | | | |
| 6680995 | cytochrome c oxidase, subunit VIIIb; COX VIII-H [Mus | 6680995 | COXQ_MOUSE | | |
| | musculus] | | | | |
| 16758308 | cytochrome c oxidase, subunit XVII assembly protein | | | | Q14061 |
| | homolog [Rattus norvegicus] | | | | |
| 6681095 | cytochrome c, somatic [Mus musculus] | 6753560 | CCMS CCMST | 11128019 | CCHU |
| 13385006 | cytochrome c-1 [Mus musculus] | | | 21359867 | S00680 |
| 231896 | Cytochrome P450 11B2, mitochondrial precursor | | | 13904853 | B34181 S11338 |
| | (CYPXIB2) (P450C11) (Steroid | | | | |
| 20867579 | cytochrome P450, 40 (25-hydroxyvitamin D3 1 alpha- | 20867579 | | 4503213 | |
| | hydroxylase) [Mus musculus] | | | | |
| 9789921 | cytochrome P450, family 11, subfamily a, polypeptide 1; | 9789921 | | 4503189 | A25922 S14367 |
| | cytochrome P450, 11a, | | | | |
| 7106287 | cytochrome P450, family 11, subfamily b, polypeptide 2; | 7106287 | A41552 | | |
| 6681097 | cytochrome P450, family 17, subfamily a, polypeptide 1; | | | | |
| | cytochrome P450, 17; | | | | |
| 6753572 | cytochrome P450, family 24, subfamily a, polypeptide 1; | 6753572 | S60033 | | A47436 |
| | cytochrome P450, 24; | | | | |
| 30578401 | cytochrome P450, family 27, subfamily a, polypeptide 1; | | | 4503211 | A39740 |
| | cytochrome P450, 27; | | | | |
| 18875324 | DAZ associated protein 1 [Mus musculus] | 18875324 | | | |
| 17505907 | DEAD (Asp-Glu-Ala-Asp) box polypeptide 31 isoform 1; | | | | |
| | DEAD/DEXH helicase DDX31 | | | | |
| 20587962 | demethyl-Q 7 [Mus musculus] | | | 25453484 | |
| 7304999 | deoxyguanosine kinase [Mus musculus] | 7304999 | | 18426967 | JC6142 |
| | | | | 18426963 | |
| | | | | 18426969 | |
| | | | | 18426965 | |
| 21281687 | deoxyuridine triphosphatase [Mus musculus] | | | 4503423 | DUT_HUMAN |
| 19745150 | diaphorase 1 (NADH) [Mus musculus] | | | | RDHUB5 |
| 6681137 | diazepam binding inhibitor; acyl-CoA binding protein; | | | | |
| | diazepam-binding inhibitor | | | | |
| 6753610 | dihydrolipoamide branched chain transacylase E2; | 6753610 | S65760 | 4503265 | A32422 |
| | BCKAD E2 [Mus musculus] | | | | |
| 31982856 | dihydrolipoamide dehydrogenase [Mus musculus] | 6681189 | 107450 | 4557525 | DEHULP |
| 31542559 | dihydrolipoamide S-acetyltransferase (E2 component of | | | 21630255 | S25665 XXHU |
| | pyruvate dehydrogenase | | | | |
| 21313536 | dihydrolipoamide S-succinyltransferase (E2 component | 21313536 | | | PN0673 |
| | of 2-oxo-glutarate complex) | | | | |
| 9910194 | dihydroorotate dehydrogenase [Mus musculus] | 9910194 | | 16753223 | PC1219 |
| 6753676 | dihydropyrimidinase-like 2; collapsin response mediator | | | | |
| | protein 2 [Mus musculus] | | | | |
| 21311901 | dimethylglycine dehydrogenase precursor [Mus | | | 24797151 | M2GD_HUMAN |
| | musculus] | | | | |
| 34328271 | direct IAP binding protein with low PI [Mus musculus] | 12963593 | | 9845297 | |
| | | | | 21070978 | |
| | | | | 21070976 | |
| 34328379 | D-lactate dehydrogenase [Mus musculus] | | | | |
| 19527228 | DNA segment, Chr 10, ERATO Doi 214, expressed | | | | |
| | [Mus musculus] | | | | |
| 20070420 | DNA segment, Chr 10, Johns Hopkins University 81 | | | | JC4913 JC4914 |
| | expressed [Mus musculus] | | | | |
| 25092662 | DNA segment, Chr 11, Wayne State University 68, | | | | |
| | expressed [Mus musculus] | | | | |
| 27552760 | DNA segment, Chr 16, Indiana University Medical 22, | 27552760 | | | |
| | expressed [Mus musculus] | | | | |
| 14861848 | DNA segment, Chr 7, Roswell Park 2 complex, | | | | |
| | expressed; androgen regulated gene | | | | |
| 31560085 | DnaJ (Hsp40) homolog, subfamily A, member 3 [Mus | 13994155 | | | |
| | musculus] | 25053902 | | | |
| 31981810 | dodecenoyl-Coenzyme A delta isomerase (3,2 trans- | 6753612 | S38770 | 4503267 | A55723 |
| | enoyl-Coenyme A isomerase) [Mus | | | | |
| 31981826 | electron transferring flavoprotein, alpha polypeptide; | | | 4503607 | A31998 |
| | Alpha-ETF [Mus musculus] | | | | |
| 21313290 | electron transferring flavoprotein, dehydrogenase [Mus | | | | Q16134 |
| | musculus] | | | | |
| 6679647 | endonuclease G [Mus musculus] | 6679647 | | 4758270 | NUCG_HUMAN |
| 19923857 | endothelial cell growth factor 1; thymidine | | | | P19971 |
| | phosphorylase; gliostatin; platelet | | | | |
| 7949037 | enoyl coenzyme A hydratase 1, peroxisomal; | 7949037 | | | |
| | peroxisomal/mitochondrial dienoyl-CoA | | | | |
| 29789289 | enoyl Coenzyme A hydratase, short chain, 1, | | | 12707570 | ECHM_HUMAN |
| | mitochondrial [Mus musculus] | | | | |
| 7305125 | estradiol 17 beta-dehydrogenase 8; 17-beta- | | | | |
| | hydroxysteroid dehydrogenase 8; | | | | |
| 18079334 | ethanol induced 6 [Mus musculus] | | | | |
| 6679078 | expressed in non-metastatic cells 2, protein; expressed | | | | |
| | in non-metastatic cells | | | | |
| 9790123 | expressed in non-metastatic cells 4, protein; nucleoside | 9790123 | | 4826862 | NDKM_HUMAN |
| | diphosphate kinase | | | | |
| 21618729 | Facl5 protein [Mus musculus] | | | | |
| 31560705 | fatty acid Coenzyme A ligase, long chain 2; acetyl- | | | | LCFA_HUMAN |
| | Coenzyme A synthetase; | | | | JX0202 |
| 6679765 | ferredoxin 1; ADRENODOXIN [Mus musculus] | 6679765 | S53524 | 4758352 | AXHU |
| 6679767 | ferredoxin reductase [Mus musculus] | 6679767 | S60028 | 4758354 | A40487 |
| | | | | 13435350 | |
| 13385780 | ferritin heavy chain 3; mitochondrial ferritin [Mus | 13385780 | | | |
| | musculus] | | | | |
| 20452466 | ferrochelatase [Mus musculus] | 20452466 | A37972 | | A36403 |
| 10946808 | fibroblast growth factor (acidic) intracellular binding | | | 7262378 | |
| | protein; aFGF | | | | |
| 33469107 | folylpolyglutamyl synthetase [Mus musculus] | 20824150 | S65755 | 22024385 | A46281 |
| 9507187 | fractured callus expressed transcript 1; Fracture Callus | 9507187 | | | |
| | 1; small zinc | | | | |
| 6679863 | frataxin [Mus musculus] | 6679863 | | 4503785 | |
| 33859554 | fumarate hydratase 1 [Mus musculus] | 20831568 | | 19743875 | UFHUM |
| 20070402 | G elongation factor; mitochondrial [Mus musculus] | | | | |
| 12963633 | genes associated with retinoid-IFN-induced mortality 19 | | | | |
| | [Mus musculus] | | | | |
| 6679957 | glioblastoma amplified [Mus musculus] | | | | |
| 31982798 | glucokinase; hexokinase 4 [Mus musculus] | | | | A46157 C46157 |
| 6680027 | glutamate dehydrogenase [Mus musculus] | 6680027 | S16239 | 27485958 | A53719 DEHUE |
| | | | | 4885281 | |
| | | | | 6912392 | |
| 6754036 | glutamate oxaloacetate transaminase 2, mitochondrial; | 6754036 | S01174 | 4504069 | XNHUDM |
| | mitochondrial aspartate | | | | |
| 31982332 | glutamate-ammonia ligase (glutamine synthase); | | | | |
| | glutamine synthetase [Mus | | | | |
| 31982847 | glutamic acid decarboxylase 1 [Mus musculus] | | | | |
| 6679959 | glutaryl-Coenzyme A dehydrogenase [Mus musculus] | 6679959 | GCDH_MOUSE | 4503943 | GCDH_HUMAN |
| | | | | 7669494 | |
| 6680075 | glutathione peroxidase 1; cellular GPx [Mus musculus] | 6680075 | | | |
| 13540480 | glutathione peroxidase 4; sperm nuclei glutathione | 13540480 | | 4504107 | |
| | peroxidase; phospholipid | | | | |
| 34328489 | glutathione reductase 1 [Mus musculus] | 13775154 | | | |
| 21313138 | glutathione S-transferase class kappa [Mus musculus] | | | | |
| 6754092 | glutathione transferase zeta 1 (maleylacetoacetate | | | | |
| | isomerase); | | | | |
| 6679937 | glyceraldehyde-3-phosphate dehydrogenase [Mus | | | | |
| | musculus] | | | | |
| 6680139 | glycerol kinase [Mus musculus] | | | | GKP2_HUMAN |
| | | | | | GLPK_HUMAN |
| 34536827 | glycerol-3-phosphate acyltransferase, mitochondrial | 6680057 | | | |
| | [Mus musculus] | | | | |
| 31981769 | glycerol-3-phosphate dehydrogenase 2; glycerol | 6753970 | | 4504085 | GPDM_HUMAN |
| | phosphate dehydrogenase 1, | | | | |
| 13385454 | glycine amidinotransferase (L-arginine: glycine | 13385454 | | 4503933 | S41734 |
| | amidinotransferase) [Mus | | | | |
| 31560488 | glycine C-acetyltransferase (2-amino-3-ketobutyrate- | 7305083 | | | |
| | coenzyme A ligase); | | | | |
| 20070408 | glycine decarboxylase [Mus musculus] | | | | B39521 |
| 6806917 | GM2 ganglioside activator protein [Mus musculus] | | | | |
| 6680107 | granulin; acrogranulin; progranulin; PC cell-derived | | | | |
| | growth factor [Mus | | | | |
| 12746414 | growth factor, erv1 (S. cerevisiae)-like (augmenter of | | | | |
| | liver regeneration); | | | | |
| 13277394 | GrpE-like 1, mitochondrial [Mus musculus] | 13277394 | | | |
| 29789124 | GrpE-like 2, mitochondrial [Mus musculus] | 20878923 | | | |
| 3766203 | GTP-specific succinyl-CoA synthetase beta subunit | 20828815 | | | T08812 |
| | [Mus musculus] | | | | |
| 2137368 | H+-transporting two-sector ATPase (EC 3.6.3.14) chain | | S58660 | | |
| | c - mouse (fragments) | | | | |
| 6680309 | heat shock protein 1 (chaperonin 10); heat shock 10 kDa | 6680309 | A55075 | | S47532 |
| | protein 1 (chaperonin | | CH10_MOUSE | | |
| 31981679 | heat shock protein 1 (chaperonin); heat shock protein, | | HHMS60 | | A32800 |
| | 60 kDa; heat shock 60 kDa | | | | |
| 6680305 | heat shock protein 1, beta; heat shock protein, 84 kDa | | | | |
| | 1; heat shock 90 kDa | | | | |
| 31560686 | heat shock protein 2; heat shock protein, 70 kDa 2; heat | | | | B45871 |
| | shock 70 kDa protein 2 | | | | |
| 6754256 | heat shock protein, A; heat shock protein cognate 74; | 6754256 | A48127 | 24234688 | B48127 |
| | heat shock protein, 74 | 25024532 | | | |
| 6680277 | heat-responsive protein 12 [Mus musculus] | | | | |
| 7305137 | heme binding protein 1; heme-binding protein; p22 | | | | |
| | HBP; heme-binding protein 1 | | | | |
| 6680175 | hemoglobin alpha, adult chain 1; alpha 1 globin [Mus | | | | |
| | musculus] | | | | |
| 122513 | Hemoglobin beta-1 chain (B1) (Major) | | | | |
| 31982300 | hemoglobin, beta adult major chain; beta major globin; | | | | |
| | beta maj [Mus musculus] | | | | |
| 6754206 | hexokinase 1; downeast anemia [Mus musculus] | | A35244 | | A31869 JC2025 |
| 20982837 | holocarboxylase synthetase; biotin- [propriony- | | | | BPL1_HUMAN |
| | Coenzyme A-carboxylase | | | | |
| 31542950 | holocytochrome c synthetase [Mus musculus] | 6680181 | CCHL_MOUSE | | G02133 |
| 6754160 | HS1 binding protein [Mus musculus] | 6754160 | | 13435356 | |
| 12963539 | HSCO protein [Mus musculus] | | | | |
| 7949047 | hydroxyacyl-Coenzyme A dehydrogenase type II; | | | | |
| | hydroxyacyl-Coenzyme A | | | | |
| 21704100 | hydroxyacyl-Coenzyme A dehydrogenase/3-ketoacyl- | | | 4504327 | JC2109 |
| | Coenzyme A | | | | |
| 33859811 | hydroxyacyl-Coenzyme A dehydrogenase/3-ketoacyl- | | | 20127408 | JC2108 |
| | Coenzyme A | | | | |
| 31982273 | hydroxysteroid (17-beta) dehydrogenase 4; | | | | |
| | hydroxysteroid 17-beta dehydrogenase | | | | |
| 6680291 | hydroxysteroid dehydrogenase-4, delta-3-beta; 3-beta- | 20874991 | I49762 | | DEHUHS DEHUH2 |
| | hydroxysteroid | 23397415 | 3BH3_MOUSE | | |
| | | 23621517 | 3BH4_MOUSE | | |
| | | 6680289 | 3BH5_MOUSE | | |
| | | 6680291 | 3BH6_MOUSE | | |
| | | 6680293 | 3BH2_MOUSE | | |
| | | 7305167 | | | |
| | | 25046137 | | | |
| 27754071 | hypothetical protein 4833421E05Rik [Mus musculus] | | | | |
| 21311867 | hypothetical protein D11Ertd99e [Mus musculus] | | | | |
| 21312020 | hypothetical protein D4Ertd765e [Mus musculus] | | | | |
| 22122743 | hypothetical protein MGC37245 [Mus musculus] | | | | |
| 21313262 | inner membrane protein, mitochondrial [Mus musculus] | | | | |
| 22203753 | inorganic pyrophosphatase 2 [Mus musculus] | | | | |
| 14916467 | inositol polyphosphate-5-phosphatase E; inositol | 14916467 | | | |
| | polyphosphate-5-phosphatase, 72 | | | | |
| 27370516 | isocitrate dehydrogenase 2 (NADP+), mitochondrial | 6680343 | IDHP_MOUSE | 4504575 | S57499 |
| | [Mus musculus] | | | | |
| 18250284 | isocitrate dehydrogenase 3 (NAD+) alpha [Mus | | | 5031777 | S55282 |
| | musculus] | | | | |
| 6680345 | isocitrate dehydrogenase 3 (NAD+), gamma [Mus | 6680345 | | | IDHG_HUMAN |
| | musculus] | | | | |
| 18700024 | isocitrate dehydrogenase 3, beta subunit; isocitrate | | | 5901982 | IDHB_HUMAN |
| | dehydrogenase 3 beta; N14A | | | | |
| 9789985 | isovaleryl coenzyme A dehydrogenase; isovaleryl | | | 4504799 | A37033 |
| | dehydrogenase precursor [Mus | | | | |
| 6754482 | keratin complex 1, acidic, gene 18; keratin 18 [Mus | | | | |
| | musculus] | | | | |
| 6754488 | keratin complex 2, basic, gene 6b [Mus musculus] | | | | |
| 19482166 | kidney expressed gene 1 [Mus musculus] | | | | |
| 25031694 | kinesin family member 1B [Mus musculus] | 20850523 | | | |
| | | 25031694 | | | |
| 19527030 | kynurenine 3-monooxygenase (kynurenine 3- | | | | |
| | hydroxylase) [Mus musculus] | | | | |
| 6754408 | kynurenine aminotransferase II [Mus musculus] | | | | |
| 6680163 | L-3-hydroxyacyl-Coenzyme A dehydrogenase, short | 6680163 | JC4210 | 4885387 | JC4879 |
| | chain; hydroxylacyl-Coenzyme A | | | | |
| 21703764 | lactamase, beta 2 [Mus musculus] | | | | |
| 13507666 | lactamase, beta; serine beta lactamase-like protein; | 13507666 | | | |
| | mitochondrial ribosomal | | | | |
| 31981147 | leucine aminopeptidase 3; leucine aminopeptidase [Mus | | | | |
| | musculus] | | | | |
| 9789997 | leucine zipper-EF-hand containing transmembrane | | | | |
| | protein 1; leucine | | | | |
| 21389320 | leucine-rich PPR motif-containing protein; leucine rich | | | | |
| | protein LRP130 [Mus | | | | |
| 23346617 | leucyl-tRNA synthetase [Mus musculus] | | | | SYLM_HUMAN |
| 13277380 | lipoic acid synthetase [Mus musculus] | 13277380 | | | |
| 6678716 | low density lipoprotein receptor-related protein 5; low | | | | |
| | density | | | | |
| 21539585 | low molecular mass ubiquinone-binding protein; | 21539585 | | | |
| | ubiquinol-cytochrome c reductase | | | | |
| 31541815 | L-specific multifunctional beta-oxdiation protein [Mus | | | | |
| | musculus] | | | | |
| 6678760 | lysophospholipase 1; phospholipase 1a; | | | | |
| | lysophopholipase 1 [Mus musculus] | | | | |
| 8393739 | lysozyme [Mus musculus] | | | | |
| 13654245 | major urinary protein 1 [Mus musculus] | | | | |
| 31982186 | malate dehydrogenase, mitochondrial [Mus musculus] | 6678916 | DEMSMM | | MDHM_HUMAN |
| 21703972 | malic enzyme 2, NAD(+)-dependent, mitochondrial | | | 4505145 | A39503 |
| | [Mus musculus] | | | | |
| 31542169 | malic enzyme 3, NADP(+)-dependent, mitochondrial | | | | S53351 |
| | [Mus musculus] | | | | |
| 9910434 | malonyl-CoA decarboxylase [Mus musculus] | | | 6912498 | DCMC_HUMAN |
| 6754760 | mature T-cell proliferation 1 [Mus musculus] | 6754760 | | | |
| 7305291 | metaxin 1; metaxin [Mus musculus] | 7305291 | | | MTXN_HUMAN |
| 31543274 | metaxin 2 [Mus musculus] | 7949084 | | | |
| 31981013 | methionine sulfoxide reductase A [Mus musculus] | | | | |
| 31980706 | methylcrotonoyl-Coenzyme A carboxylase 1 (alpha) | 12965187 | | | |
| | [Mus musculus] | | | | |
| 6678952 | methylenetetrahydrofolate dehydrogenase (NAD+ | 6678952 | A33267 | 5729935 | DEHUMT |
| | dependent), | | | | |
| 20270275 | methylenetetrahydrofolate dehydrogenase 1; C1- | | | 13699868 | A31903 |
| | tetrahydrofolate synthase [Mus | | | | |
| 6678970 | methylmalonyl-Coenzyme A mutase [Mus musculus] | 6678970 | S08680 | 4557767 | S40622 |
| 31981068 | microsomal glutathione S-transferase 1 [Mus musculus] | | | | B28083 |
| 30794474 | mitchondrial ribosomal protein S7; ribosomal protein, | | | | JC7165 |
| | mitochondrial, S7 [Mus | | | | |
| 19527402 | mitochondrial acyl-CoA thioesterase 1 [Mus musculus] | 19527402 | | | |
| 13386040 | mitochondrial ATP synthase regulatory component | 13386040 | | | |
| | factor B [Mus musculus] | | | | |
| 15011842 | mitochondrial capsule selenoprotein; sperm | 15011842 | A37199 | | MCS_HUMAN |
| | mitochondria associated cysteine-rich | | | | |
| 9790055 | mitochondrial carrier homolog 2 [Mus musculus] | | | | |
| 28076953 | mitochondrial intermediate peptidase [Mus musculus] | | | 5174567 | |
| 27502349 | mitochondrial matrix processing protease, alpha subunit | | | | Q10713 |
| | [Mus musculus] | | | | |
| 31559891 | mitochondrial Rho 1 [Mus musculus] | | | | |
| 22164792 | mitochondrial ribosomal protein L12 [Mus musculus] | | | | RM12_HUMAN |
| | phosphorylase; gliostatin; platelet | | | | |
| 7949037 | enoyl coenzyme A hydratase 1, peroxisomal; | 7949037 | | | |
| | peroxisomal/mitochondrial dienoyl-CoA | | | | |
| 29789289 | enoyl Coenzyme A hydratase, short chain, 1, | | | 12707570 | ECHM_HUMAN |
| | mitochondrial [Mus musculus] | | | | |
| 7305125 | estradiol 17 beta-dehydrogenase 8; 17-beta- | | | | |
| | hydroxysteroid dehydrogenase 8; | | | | |
| 18079334 | ethanol induced 6 [Mus musculus] | | | | |
| 6679078 | expressed in non-metastatic cells 2, protein; expressed | | | | |
| | in non-metastatic cells | | | | |
| 9790123 | expressed in non-metastatic cells 4, protein; nucleoside | 9790123 | | 4826862 | NDKM_HUMAN |
| | diphosphate kinase | | | | |
| 21618729 | Facl5 protein [Mus musculus] | | | | |
| 31560705 | fatty acid Coenzyme A ligase, long chain 2; acetyl- | | | | LCFA_HUMAN |
| | Coenzyme A synthetase; | | | | JX0202 |
| 6679765 | ferredoxin 1; ADRENODOXIN [Mus musculus] | 6679765 | S53524 | 4758352 | AXHU |
| 6679767 | ferredoxin reductase [Mus musculus] | 6679767 | S60028 | 4758354 | A40487 |
| | | | | 13435350 | |
| 13385780 | ferritin heavy chain 3; mitochondrial ferritin [Mus | 13385780 | | | |
| | musculus] | | | | |
| 20452466 | ferrochelatase [Mus musculus] | 20452466 | A37972 | | A36403 |
| 10946808 | fibroblast growth factor (acidic) intracellular binding | | | 7262378 | |
| | protein; aFGF | | | | |
| 33469107 | folylpolyglutamyl synthetase [Mus musculus] | 20824150 | S65755 | 22024385 | A46281 |
| 9507187 | fractured callus expressed transcript 1; Fracture Callus | 9507187 | | | |
| | 1; small zinc | | | | |
| 6679863 | frataxin [Mus musculus] | 6679863 | | 4503785 | |
| 33859554 | fumarate hydratase 1 [Mus musculus] | 20831568 | | 19743875 | UFHUM |
| 20070402 | G elongation factor; mitochondrial [Mus musculus] | | | | |
| 12963633 | genes associated with retinoid-IFN-induced mortality 19 | | | | |
| | [Mus musculus] | | | | |
| 6679957 | glioblastoma amplified [Mus musculus] | | | | |
| 31982798 | glucokinase; hexokinase 4 [Mus musculus] | | | | A46157 C46157 |
| 6680027 | glutamate dehydrogenase [Mus musculus] | 6680027 | S16239 | 27485958 | A53719 DEHUE |
| | | | | 4885281 | |
| | | | | 6912392 | |
| 6754036 | glutamate oxaloacetate transaminase 2, mitochondrial; | 6754036 | S01174 | 4504069 | XNHUDM |
| | mitochondrial aspartate | | | | |
| 31982332 | glutamate-ammonia ligase (glutamine synthase); | | | | |
| | glutamine synthetase [Mus | | | | |
| 31982847 | glutamic acid decarboxylase 1 [Mus musculus] | | | | |
| 6679959 | glutaryl-Coenzyme A dehydrogenase [Mus musculus] | 6679959 | GCDH_MOUSE | 4503943 | GCDH_HUMAN |
| | | | | 7669494 | |
| 6680075 | glutathione peroxidase 1; cellular GPx [Mus musculus] | 6680075 | | | |
| 13540480 | glutathione peroxidase 4; sperm nuclei glutathione | 13540480 | | 4504107 | |
| | peroxidase; phospholipid | | | | |
| 34328489 | glutathione reductase 1 [Mus musculus] | 13775154 | | | |
| 21313138 | glutathione S-transferase class kappa [Mus musculus] | | | | |
| 6754092 | glutathione transferase zeta 1 (maleylacetoacetate | | | | |
| | isomerase); | | | | |
| 6679937 | glyceraldehyde-3-phosphate dehydrogenase [Mus | | | | |
| | musculus] | | | | |
| 6680139 | glycerol kinase [Mus musculus] | | | | GKP2_HUMAN |
| | | | | | GLPK_HUMAN |
| 34536827 | glycerol-3-phosphate acyltransferase, mitochondrial | 6680057 | | | |
| | [Mus musculus] | | | | |
| 31981769 | glycerol-3-phosphate dehydrogenase 2; glycerol | 6753970 | | 4504085 | GPDM_HUMAN |
| | phosphate dehydrogenase 1, | | | | |
| 13385454 | glycine amidinotransferase (L-arginine: glycine | 13385454 | | 4503933 | S41734 |
| | amidinotransferase) [Mus | | | | |
| 31560488 | glycine C-acetyltransferase (2-amino-3-ketobutyrate- | 7305083 | | | |
| | coenzyme A ligase); | | | | |
| 20070408 | glycine decarboxylase [Mus musculus] | | | | B39521 |
| 6806917 | GM2 ganglioside activator protein [Mus musculus] | | | | |
| 6680107 | granulin; acrogranulin; progranulin; PC cell-derived | | | | |
| | growth factor [Mus | | | | |
| 12746414 | growth factor, erv1 (S. cerevisiae)-like (augmenter of | | | | |
| | liver regeneration); | | | | |
| 13277394 | GrpE-like 1, mitochondrial [Mus musculus] | 13277394 | | | |
| 29789124 | GrpE-like 2, mitochondrial [Mus musculus] | 20878923 | | | |
| 3766203 | GTP-specific succinyl-CoA synthetase beta subunit | 20828815 | | | T08812 |
| | [Mus musculus] | | | | |
| 2137368 | H+-transporting two-sector ATPase (EC 3.6.3.14) chain | | S58660 | | |
| | c - mouse (fragments) | | | | |
| 6680309 | heat shock protein 1 (chaperonin 10); heat shock 10 kDa | 6680309 | A55075 | | S47532 |
| | protein 1 (chaperonin | | CH10_MOUSE | | |
| 31981679 | heat shock protein 1 (chaperonin); heat shock protein, | | HHMS60 | | A32800 |
| | 60 kDa; heat shock 60 kDa | | | | |
| 6680305 | heat shock protein 1, beta; heat shock protein, 84 kDa | | | | |
| | 1; heat shock 90 kDa | | | | |
| 31560686 | heat shock protein 2; heat shock protein, 70 kDa 2; heat | | | | B45871 |
| | shock 70 kDa protein 2 | | | | |
| 6754256 | heat shock protein, A; heat shock protein cognate 74; | 6754256 | A48127 | 24234688 | B48127 |
| | heat shock protein, 74 | 25024532 | | | |
| 6680277 | heat-responsive protein 12 [Mus musculus] | | | | |
| 7305137 | heme binding protein 1; heme-binding protein; p22 | | | | |
| | HBP; heme-binding protein 1 | | | | |
| 6680175 | hemoglobin alpha, adult chain 1; alpha 1 globin [Mus | | | | |
| | musculus] | | | | |
| 122513 | Hemoglobin beta-1 chain (B1) (Major) | | | | |
| 31982300 | hemoglobin, beta adult major chain; beta major globin; | | | | |
| | beta maj [Mus musculus] | | | | |
| 6754206 | hexokinase 1; downeast anemia [Mus musculus] | | A35244 | | A31869 JC2025 |
| 20982837 | holocarboxylase synthetase; biotin- [propriony- | | | | BPL1_HUMAN |
| | Coenzyme A-carboxylase | | | | |
| 31542950 | holocytochrome c synthetase [Mus musculus] | 6680181 | CCHL_MOUSE | | G02133 |
| 6754160 | HS1 binding protein [Mus musculus] | 6754160 | | 13435356 | |
| 12963539 | HSCO protein [Mus musculus] | | | | |
| 7949047 | hydroxyacyl-Coenzyme A dehydrogenase type II; | | | | |
| | hydroxyacyl-Coenzyme A | | | | |
| 21704100 | hydroxyacyl-Coenzyme A dehydrogenase/3-ketoacyl- | | | 4504327 | JC2109 |
| | Coenzyme A | | | | |
| 33859811 | hydroxyacyl-Coenzyme A dehydrogenase/3-ketoacyl- | | | 20127408 | JC2108 |
| | Coenzyme A | | | | |
| 31982273 | hydroxysteroid (17-beta) dehydrogenase 4; | | | | |
| | hydroxysteroid 17-beta dehydrogenase | | | | |
| 6680291 | hydroxysteroid dehydrogenase-4, delta-3-beta; 3-beta- | 20874991 | I49762 | | DEHUHS DEHUH2 |
| | hydroxysteroid | 23397415 | 3BH3_MOUSE | | |
| | | 23621517 | 3BH4_MOUSE | | |
| | | 6680289 | 3BH5_MOUSE | | |
| | | 6680291 | 3BH6_MOUSE | | |
| | | 6680293 | 3BH2_MOUSE | | |
| | | 7305167 | | | |
| | | 25046137 | | | |
| 27754071 | hypothetical protein 4833421E05Rik [Mus musculus] | | | | |
| 21311867 | hypothetical protein D11Ertd99e [Mus musculus] | | | | |
| 21312020 | hypothetical protein D4Ertd765e [Mus musculus] | | | | |
| 22122743 | hypothetical protein MGC37245 [Mus musculus] | | | | |
| 21313262 | inner membrane protein, mitochondrial [Mus musculus] | | | | |
| 22203753 | inorganic pyrophosphatase 2 [Mus musculus] | | | | |
| 14916467 | inositol polyphosphate-5-phosphatase E; inositol | 14916467 | | | |
| | polyphosphate-5-phosphatase, 72 | | | | |
| 27370516 | isocitrate dehydrogenase 2 (NADP+), mitochondrial | 6680343 | IDHP_MOUSE | 4504575 | S57499 |
| | [Mus musculus] | | | | |
| 18250284 | isocitrate dehydrogenase 3 (NAD+) alpha [Mus | | | 5031777 | S55282 |
| | musculus] | | | | |
| 6680345 | isocitrate dehydrogenase 3 (NAD+), gamma [Mus | 6680345 | | | IDHG_HUMAN |
| | musculus] | | | | |
| 18700024 | isocitrate dehydrogenase 3, beta subunit; isocitrate | | | 5901982 | IDHB_HUMAN |
| | dehydrogenase 3 beta; N14A | | | | |
| 9789985 | isovaleryl coenzyme A dehydrogenase; isovaleryl | | | 4504799 | A37033 |
| | dehydrogenase precursor [Mus | | | | |
| 6754482 | keratin complex 1, acidic, gene 18; keratin 18 [Mus | | | | |
| | musculus] | | | | |
| 6754488 | keratin complex 2, basic, gene 6b [Mus musculus] | | | | |
| 19482166 | kidney expressed gene 1 [Mus musculus] | | | | |
| 25031694 | kinesin family member 1B [Mus musculus] | 20850523 | | | |
| | | 25031694 | | | |
| 19527030 | kynurenine 3-monooxygenase (kynurenine 3- | | | | |
| | hydroxylase) [Mus musculus] | | | | |
| 6754408 | kynurenine aminotransferase II [Mus musculus] | | | | |
| 6680163 | L-3-hydroxyacyl-Coenzyme A dehydrogenase, short | 6680163 | JC4210 | 4885387 | JC4879 |
| | chain; hydroxylacyl-Coenzyme A | | | | |
| 21703764 | lactamase, beta 2 [Mus musculus] | | | | |
| 13507666 | lactamase, beta; serine beta lactamase-like protein; | 13507666 | | | |
| | mitochondrial ribosomal | | | | |
| 31981147 | leucine aminopeptidase 3; leucine aminopeptidase [Mus | | | | |
| | musculus] | | | | |
| 9789997 | leucine zipper-EF-hand containing transmembrane | | | | |
| | protein 1; leucine | | | | |
| 21389320 | leucine-rich PPR motif-containing protein; leucine rich | | | | |
| | protein LRP130 [Mus | | | | |
| 23346617 | leucyl-tRNA synthetase [Mus musculus] | | | | SYLM_HUMAN |
| 13277380 | lipoic acid synthetase [Mus musculus] | 13277380 | | | |
| 6678716 | low density lipoprotein receptor-related protein 5; low | | | | |
| | density | | | | |
| 21539585 | low molecular mass ubiquinone-binding protein; | 21539585 | | | |
| | ubiquinol-cytochrome c reductase | | | | |
| 31541815 | L-specific multifunctional beta-oxdiation protein [Mus | | | | |
| | musculus] | | | | |
| 6678760 | lysophospholipase 1; phospholipase 1a; | | | | |
| | lysophopholipase 1 [Mus musculus] | | | | |
| 8393739 | lysozyme [Mus musculus] | | | | |
| 13654245 | major urinary protein 1 [Mus musculus] | | | | |
| 31982186 | malate dehydrogenase, mitochondrial [Mus musculus] | 6678916 | DEMSMM | | MDHM_HUMAN |
| 21703972 | malic enzyme 2, NAD(+)-dependent, mitochondrial | | | 4505145 | A39503 |
| | [Mus musculus] | | | | |
| 31542169 | malic enzyme 3, NADP(+)-dependent, mitochondrial | | | | S53351 |
| | [Mus musculus] | | | | |
| 9910434 | malonyl-CoA decarboxylase [Mus musculus] | | | 6912498 | DCMC_HUMAN |
| 6754760 | mature T-cell proliferation 1 [Mus musculus] | 6754760 | | | |
| 7305291 | metaxin 1; metaxin [Mus musculus] | 7305291 | | | MTXN_HUMAN |
| 31543274 | metaxin 2 [Mus musculus] | 7949084 | | | |
| 31981013 | methionine sulfoxide reductase A [Mus musculus] | | | | |
| 31980706 | methylcrotonoyl-Coenzyme A carboxylase 1 (alpha) | 12965187 | | | |
| | [Mus musculus] | | | | |
| 6678952 | methylenetetrahydrofolate dehydrogenase (NAD+ | 6678952 | A33267 | 5729935 | DEHUMT |
| | dependent), | | | | |
| 20270275 | methylenetetrahydrofolate dehydrogenase 1; C1- | | | 13699868 | A31903 |
| | tetrahydrofolate synthase [Mus | | | | |
| 6678970 | methylmalonyl-Coenzyme A mutase [Mus musculus] | 6678970 | S08680 | 4557767 | S40622 |
| 31981068 | microsomal glutathione S-transferase 1 [Mus musculus] | | | | B28083 |
| 30794474 | mitchondrial ribosomal protein S7; ribosomal protein, | | | | JC7165 |
| | mitochondrial, S7 [Mus | | | | |
| 19527402 | mitochondrial acyl-CoA thioesterase 1 [Mus musculus] | 19527402 | | | |
| 13386040 | mitochondrial ATP synthase regulatory component | 13386040 | | | |
| | factor B [Mus musculus] | | | | |
| 15011842 | mitochondrial capsule selenoprotein; sperm | 15011842 | A37199 | | MCS_HUMAN |
| | mitochondria associated cysteine-rich | | | | |
| 9790055 | mitochondrial carrier homolog 2 [Mus musculus] | | | | |
| 28076953 | mitochondrial intermediate peptidase [Mus musculus] | | | 5174567 | |
| 27502349 | mitochondrial matrix processing protease, alpha subunit | | | | Q10713 |
| | [Mus musculus] | | | | |
| 31559891 | mitochondrial Rho 1 [Mus musculus] | | | | |
| 22164792 | mitochondrial ribosomal protein L12 [Mus musculus] | | | | RM12_HUMAN |
| 16716447 | mitochondrial ribosomal protein L27 [Mus musculus] | 16716447 | | | |
| 31981470 | mitochondrial ribosomal protein L3 [Mus musculus] | | | | R5HUL3 R5HUL3 |
| 13385266 | mitochondrial ribosomal protein L33 [Mus musculus] | | | | |
| 16716449 | mitochondrial ribosomal protein L34 [Mus musculus] | 16716449 | | | |
| 31560438 | mitochondrial ribosomal protein L39; ribosomal protein, | 8393021 | | | |
| | mitochondrial, L5 [Mus | | | | |
| 13385752 | mitochondrial ribosomal protein L49; neighbor of fau 1 | 13385752 | | | |
| | [Mus musculus] | | | | |
| 30519921 | mitochondrial ribosomal protein L50 [Mus musculus] | | | | |
| 29789253 | mitochondrial ribosomal protein L9 [Mus musculus] | 20874698 | | | |
| 17157979 | mitochondrial ribosomal protein S11 [Mus musculus] | 17157979 | | | |
| 6755360 | mitochondrial ribosomal protein S12; ribosomal protein, | 6755360 | | | RT12_HUMAN |
| | mitochondrial, S12; | | | | |
| 13384894 | mitochondrial ribosomal protein S14 [Mus musculus] | | | | |
| 13384968 | mitochondrial ribosomal protein S15 [Mus musculus] | 13384968 | | | |
| 13384844 | mitochondrial ribosomal protein S16 [Mus musculus] | 13384844 | | | |
| 13384854 | mitochondrial ribosomal protein S17 [Mus musculus] | 13384854 | | | |
| 31543265 | mitochondrial ribosomal protein S2 [Mus musculus] | | | | |
| 17505220 | mitochondrial ribosomal protein S21 [Mus musculus] | 17505220 | | | |
| 31981257 | mitochondrial ribosomal protein S25 [Mus musculus] | 13385024 | | | |
| 10181116 | mitochondrial ribosomal protein S31; islet mitochondrial | 10181116 | | 5031787 | |
| | antigen, 38 kD [Mus | | | | |
| 17157985 | mitochondrial ribosomal protein S5 [Mus musculus] | | | | |
| 23956244 | mitochondrial ribosomal protein S6 [Mus musculus] | 23956244 | | | |
| 19526984 | mitochondrial translational initiation factor 2 [Mus | | | 4505277 | A55628 |
| | musculus] | | | | |
| 31981857 | mitochondrial translational release factor 1 [Mus | | | 4758744 | RF1M_HUMAN |
| | musculus] | | | | |
| 27804325 | monoamine oxidase A [Mus musculus] | 20983270 | I48342 | | A36175 |
| | | 27804325 | | | |
| 19073795 | MTO1 [Mus musculus] | 19073795 | | | |
| 6754732 | myeloperoxidase [Mus musculus] | | | | OPHUM |
| 22003874 | N-acetylglutamate synthase; amino-acid N- | 22003874 | | | |
| | acetyltransferase [Mus musculus] | | | | |
| 9055168 | N-acylsphingosine amidohydrolase 2; neutral/alkaline; | | | 9845267 | |
| | neutral/alkaline | | | | |
| 13195624 | NADH dehydrogenase (ubiquinone) 1 alpha | | | | O95299 |
| | subcomplex 10 [Mus musculus] | | | | |
| 9506911 | NADH dehydrogenase (ubiquinone) 1 alpha | 9506911 | | | O15239 |
| | subcomplex, 1 (7.5 kD, MWFE); NADH | | | | |
| 31981600 | NADH dehydrogenase (ubiquinone) 1 alpha | | | | O43678 |
| | subcomplex, 2; NADH dehydrogenase | | | | |
| 33563266 | NADH dehydrogenase (ubiquinone) 1 alpha | | NUML_MOUSE | | NUML_HUMAN |
| | subcomplex, 4; NADH dehydrogenase | | | | |
| 13386100 | NADH dehydrogenase (ubiquinone) 1 alpha | | | | NUFM_Human |
| | subcomplex, 5 [Mus musculus] | | | | |
| 13385492 | NADH dehydrogenase (ubiquinone) 1 alpha | | | | P56556 |
| | subcomplex, 6 (B14); NADH dehydrogenase | | | | |
| 12963571 | NADH dehydrogenase (ubiquinone) 1 alpha | | | | AAD05427 |
| | subcomplex, 7 (B14.5a); NADH | | | | |
| 21312012 | NADH dehydrogenase (ubiquinone) 1 alpha | | | 7657369 | NUPM_HUMAN |
| | subcomplex, 8 [Mus musculus] | | | | |
| 13384720 | NADH dehydrogenase (ubiquinone) 1 alpha | | | | NUEM_HUMAN |
| | subcomplex, 9 [Mus musculus] | | | | |
| 31980802 | NADH dehydrogenase (ubiquinone) 1 alpha | 27229088 | | | |
| | subcomplex, assembly factor 1; NADH | | | | |
| 13385054 | NADH dehydrogenase (ubiquinone) 1 beta subcomplex | | | 4505361 | O43676 |
| | 3 [Mus musculus] | | | | |
| 13385558 | NADH dehydrogenase (ubiquinone) 1 beta subcomplex | | | | JE0382 |
| | 8 [Mus musculus] | | | | |
| 13386096 | NADH dehydrogenase (ubiquinone) 1 beta subcomplex, | | | | |
| | 2 [Mus musculus] | | | | |
| 27754144 | NADH dehydrogenase (ubiquinone) 1 beta subcomplex, | | | | O43674 |
| | 5; NADH dehydrogenase | | | | |
| 13385322 | NADH dehydrogenase (ubiquinone) 1 beta subcomplex, | | | | NB8M_HUMAN |
| | 7 [Mus musculus] | | | | |
| 29789148 | NADH dehydrogenase (ubiquinone) 1 beta subcomplex, | | | | |
| | 9 [Mus musculus] | | | | |
| 27754007 | NADH dehydrogenase (ubiquinone) 1, alpha/beta | | | | T00741 |
| | subcomplex, 1 [Mus musculus] | | | | |
| 13384946 | NADH dehydrogenase (ubiquinone) 1, subcomplex | | | | O43677 |
| | unknown, 1 [Mus musculus] | | | | |
| 21704020 | NADH dehydrogenase (ubiquinone) Fe—S protein 1 | | | | S17854 |
| | [Mus musculus] | | | | |
| 23346461 | NADH dehydrogenase (ubiquinone) Fe—S protein 2; | | | | JE0193 |
| | NADH-coenzyme Q reductase [Mus | | | | |
| 6754814 | NADH dehydrogenase (ubiquinone) Fe—S protein 4; | | | | NUYM_HUMAN |
| | NADH dehydrogenase (ubiquinone) | | | | |
| 19527334 | NADH dehydrogenase (ubiquinone) Fe—S protein 5; | | | | O43920 |
| | NADH dehydrogenase Fe—S protein | | | | |
| 21312950 | NADH dehydrogenase (ubiquinone) Fe—S protein 7 | | | | O75251 |
| | [Mus musculus] | | | | |
| 21450107 | NADH dehydrogenase (ubiquinone) Fe—S protein 8 | | | | NUIM_HUMAN |
| | [Mus musculus] | | | | |
| 19526814 | NADH dehydrogenase (ubiquinone) flavoprotein 1; | | | | A44362 |
| | NADH dehydrogenase flavoprotein | | | | |
| 20900762 | NADH dehydrogenase (ubiquinone) flavoprotein 2 [Mus | 20900762 | | | A30113 |
| | musculus] | | | | |
| 5834954 | NADH dehydrogenase subunit 1 [Mus musculus] | 5834954 | QXMS1M | | DNHUN1 |
| 5834955 | NADH dehydrogenase subunit 2 [Mus musculus] | 5834955 | QXMS2M | | DNHUN2 |
| 5834961 | NADH dehydrogenase subunit 3 [Mus musculus] | 5834961 | QXMS3M | | DNHUN3 |
| 5834963 | NADH dehydrogenase subunit 4 [Mus musculus] | 5834963 | QXMS4M | | DNHUN4 |
| 5834962 | NADH dehydrogenase subunit 4L [Mus musculus] | 5834962 | QXMS4L | | DNHUNL |
| 7770109 | NADH dehydrogenase subunit 5 [Mus musculus | | | | DNHUN5 |
| | domesticus] | | | | |
| 5834964 | NADH dehydrogenase subunit 5 [Mus musculus] | 5834964 | QXMS5M | | |
| 5834965 | NADH dehydrogenase subunit 6 [Mus musculus] | 5834965 | DEMSN6 | 27754188 | DEHUN6 |
| 21314826 | NADH: ubiquinone oxidoreductase B15 subunit [Mus | | | | O95168 |
| | musculus] | | | | |
| 21539587 | NADH-ubiquinone oxidoreductase B9 subunit; Complex | 21539587 | | | O95167 |
| | I-B9; Cl-B9 [Mus musculus] | | | | |
| 13507612 | NADPH-dependent retinol dehydrogenase/reductase | | | | |
| | [Mus musculus] | | | | |
| 6754870 | neighbor of Cox4 [Mus musculus] | | | 5174615 | |
| 200022 | neurofilament protein | | | | |
| 9506933 | neuronal protein 15.6 [Mus musculus] | | | | |
| 31543330 | nicotinamide nucleotide transhydrogenase [Mus | 6679088 | S54876 | | G02257 |
| | musculus] | | | | |
| 13385084 | NIPSNAP-related protein [Mus musculus] | | | | |
| 12963555 | Nit protein 2 [Mus musculus] | | | | |
| 21313484 | nitrogen fixation cluster-like [Mus musculus] | | | | |
| 6754846 | nitrogen fixation gene, yeast homolog 1; nifS-like (sic) | 25058437 | | 26006849 | |
| | [Mus musculus] | 6754846 | | | |
| 6679146 | nth (endonuclease III)-like 1; thymine glycol DNA | 6679146 | | | |
| | glycosylase/AP lyase [Mus | | | | |
| 31543343 | nuclear respiratory factor 1 [Mus musculus] | | | | A54868 |
| 27753998 | nudix (nucleoside diphosphate linked moiety X)-type | 27753998 | | | |
| | motif 9 [Mus musculus] | | | | |
| 19526960 | optic atrophy 1 homolog [Mus musculus] | 19526960 | | | T00336 |
| 8393866 | ornithine aminotransferase [Mus musculus] | 8393866 | XNMSO | 4557809 | XNHUO |
| 6679184 | ornithine transcarbamylase; sparse fur [Mus musculus] | 6679184 | OWMS | 9257234 | OWHU |
| 33563270 | oxoglutarate dehydrogenase (lipoamide); alpha- | 20853413 | I48884 | | A38234 |
| | ketoglutarate dehydrogenase [Mus | 25025547 | ODO1_MOUSE | | |
| 11528520 | p53 apoptosis effector related to Pmp22; p53 apoptosis- | 11528520 | | | |
| | associated target [Mus | | | | |
| 19527310 | peptidylprolyl isomerase F (cyclophilin F); peptidyl-prolyl | 19527310 | | | A41581 |
| | cis-trans isomerase; | | | | |
| 6680690 | peroxiredoxin 3; anti-oxidant protein 1; mitochondrial | 6680690 | JQ0064 | | TDXM_HUMAN |
| | Trx dependent peroxide | | | | |
| 7948999 | peroxiredoxin 4; antioxidant enzyme AOE372; Prx IV | | | | |
| | [Mus musculus] | | | | |
| 6755114 | peroxiredoxin 5 precursor; peroxiredoxin 6; peroxisomal | 6755114 | | 6912238 | |
| | membrane protein 20; | | | | |
| 18875408 | peroxisomal acyl-CoA thioesterase 1 [Mus musculus] | | | | |
| 31980804 | peroxisomal trans 2-enoyl CoA reductase; perosisomal | | | | |
| | 2-enoyl-CoA reductase [Mus | | | | |
| 21450279 | PET112-like [Mus musculus] | | | 4758894 | GATB_HUMAN |
| 10946832 | phorbol-12-myristate-13-acetate-induced protein 1; | 10946832 | | | |
| | Noxa protein [Mus musculus] | | | | |
| 33667036 | phosphatidylethanolamine N-methyltransferase [Mus | 7110685 | | | PEMT_HUMAN |
| | musculus] | | | | |
| 6755090 | phospholipase A2, group IB, pancreas [Mus musculus] | | | | PSHU |
| 7242175 | phospholipase A2, group IIA (platelets, synovial fluid); | | I48342 | | |
| | modifier of Min1; | | | | |
| 6679369 | phospholipase A2, group IVA (cytosolic, calcium- | | | | A39329 |
| | dependent); phospholipase A2, | | | | |
| 7657467 | polymerase (DNA directed), gamma 2, accessory | 7657467 | | | |
| | subunit; mitochondrial polymerase | | | | |
| 8567392 | polymerase (DNA directed), gamma; polymerase, | 8567392 | DPOG_MOUSE | 4505937 | G02750 |
| | gamma; Pol gamma; polymerase | | | | |
| 14780884 | polymerase delta interacting protein 38 [Mus musculus] | | | | |
| 6755004 | programmed cell death 8; programmed cell death 8 | 6755004 | | 4757732 | |
| | (apoptosis inducing factor); | | | 22202629 | |
| | | | | 22202631 | |
| 6679299 | prohibitin [Mus musculus] | | | | |
| 6755178 | proline dehydrogenase [Mus musculus] | 25053948 | | | |
| | | 6755178 | | | |
| 13385310 | propionyl Coenzyme A carboxylase, beta polypeptide | | | 4557044 | A53020 |
| | [Mus musculus] | | | | |
| 21450241 | propionyl-Coenzyme A carboxylase, alpha polypeptide; | | | 4557833 | A27883 |
| | propionyl CoA-carboxylase | | | | |
| 34328185 | prosaposin [Mus musculus] | | | | |
| 31980991 | protease, serine, 25; serine protease OMI [Mus | 9790135 | | | |
| | musculus] | | | | |
| 6679437 | protective protein for beta-galactosidase [Mus | | | | |
| | musculus] | | | | |
| 6679445 | protoporphyrinogen oxidase [Mus musculus] | 6679445 | S68367 | 4506001 | PPOX_HUMAN |
| 21553115 | putative mitochondrial solute carrier [Mus musculus] | 21553115 | | | |
| 31543280 | putative prostate cancer tumor suppressor; cDNA | | | | |
| | sequence BC003311 [Mus musculus] | | | | |
| 21450149 | pyrroline-5-carboxylate reductase 1; hypothetical | | | | A41770 |
| | protein MGC11688 [Mus | | | | |
| 24025659 | pyrroline-5-carboxylate synthetase; glutamate gamma- | 9790061 | | | |
| | semialdehyde synthetase [Mus | 24025659 | | | |
| 6679237 | pyruvate carboxylase; pyruvate decarboxylase [Mus | 6679237 | A47255 | 11761615 | JC2460 |
| | musculus] | | | 4505627 | |
| 18152793 | pyruvate dehydrogenase (lipoamide) beta [Mus | | | 4505687 | DEHUPB |
| | musculus] | | | | |
| 28201978 | pyruvate dehydrogenase complex, component X; | | | 4505699 | |
| | dihydrolipoamide | | | | |
| 6679261 | pyruvate dehydrogenase E1 alpha 1; pyruvate | 6679263 | S23507 S23506 | 4505685 | DEHUPA DEHUPT |
| | dehydrogenase E1alpha subunit [Mus | 6679261 | | | |
| 19526816 | pyruvate dehydrogenase kinase, isoenzyme 2; pyruvate | 19526816 | | | I70159 |
| | dehydrogenase 2 [Mus | | | | |
| 21704122 | pyruvate dehydrogenase kinase, isoenzyme 3 [Mus | | | 4885545 | I70160 |
| | musculus] | | | | |
| 7305375 | pyruvate dehydrogenase kinase, isoenzyme 4; pyruvate | 7305375 | | 4505693 | Q16654 |
| | dehydrogenase kinase 4 [Mus | | | | |
| 31981562 | pyruvate kinase 3 [Mus musculus] | | | | |
| 31543608 | reticulon 4 interacting protein 1; NOGO-interacting | 18700036 | | | |
| | mitochondrial protein; | | | | |
| 22267464 | retinoic acid inducible protein 3 [Mus musculus] | | | | |
| 6755334 | ribonuclease H1 [Mus musculus] | | | | |
| 12584986 | ribosomal protein L23 [Mus musculus] | | | | RL23_HUMAN |
| 13384904 | ribosomal protein, mitochondrial, S22 [Mus musculus] | 13384904 | | | |
| 21311883 | RIKEN cDNA 0610007O07 [Mus musculus] | | | | |
| 21311967 | RIKEN cDNA 0610008C08 [Mus musculus] | | | | |
| 21536220 | RIKEN cDNA 0610008F14 [Mus musculus] | | | | S22348 |
| 21313679 | RIKEN cDNA 0610009D10 [Mus musculus] | | | | |
| 21312004 | RIKEN cDNA 0610009I16 [Mus musculus] | | | | S32482 |
| 13385656 | RIKEN cDNA 0610010D20 [Mus musculus] | | | | |
| 21311853 | RIKEN cDNA 0610012H03 [Mus musculus] | | | | |
| 21313618 | RIKEN cDNA 0610041L09 [Mus musculus] | | | | |
| 13385662 | RIKEN cDNA 0610042E07 [Mus musculus] | | | | |
| 27754146 | RIKEN cDNA 0710001P09 [Mus musculus] | | | | |
| 21312028 | RIKEN cDNA 1110006I11 [Mus musculus] | | | | |
| 13384742 | RIKEN cDNA 1110018B13 [Mus musculus] | | | | |
| 13384766 | RIKEN cDNA 1110021D01 [Mus musculus] | | | | |
| 12963697 | RIKEN cDNA 1110025H10 [Mus musculus] | | | | |
| 13385298 | RIKEN cDNA 1300002A08 [Mus musculus] | 13385298 | | | |
| 21311845 | RIKEN cDNA 1300006L01 [Mus musculus] | 21311845 | | | |
| 33859744 | RIKEN cDNA 1500032D16 [Mus musculus] | | | | NUOM_HUMAN |
| 18859597 | RIKEN cDNA 1810004I06 [Mus musculus] | | | | O95298 |
| 20876012 | RIKEN cDNA 1810020M02 [Mus musculus] | | | | I38079 |
| 20897872 | RIKEN cDNA 1810058I14 [Mus musculus] | | | | |
| 21624609 | RIKEN cDNA 2010012D11 [Mus musculus] | | | | |
| 13385436 | RIKEN cDNA 2010100O12 [Mus musculus] | | | | |
| 21312554 | RIKEN cDNA 2010107E04 [Mus musculus] | 21312554 | P56379 | | 68MP_HUMAN |
| 13385042 | RIKEN cDNA 2010309E21 [Mus musculus] | | | | |
| 27370092 | RIKEN cDNA 2300002G02 [Mus musculus] | | PD0441 | 21359837 | I53499 S62767 |
| 31980955 | RIKEN cDNA 2310005D12 [Mus musculus] | | | | |
| 33859690 | RIKEN cDNA 2310005O14 [Mus musculus] | | | | |
| 21312348 | RIKEN cDNA 2310020P08 [Mus musculus] | 21312348 | | | |
| 13384950 | RIKEN cDNA 2310039H17 [Mus musculus] | | | | |
| 21313468 | RIKEN cDNA 2310050B20 [Mus musculus] | 21313468 | | 21361280 | I84606 |
| 13385998 | RIKEN cDNA 2410002K23 [Mus musculus] | 13385998 | | | |
| 31560255 | RIKEN cDNA 2410005O16 [Mus musculus] | | | | |
| 27228985 | RIKEN cDNA 2410011G03 [Mus musculus] | | | | |
| 30794396 | RIKEN cDNA 2410021P16 [Mus musculus] | | | | |
| 21312594 | RIKEN cDNA 2610205H19; EST AA108335 [Mus | | | | |
| | musculus] | | | | |
| 13195670 | RIKEN cDNA 2610207I16 [Mus musculus] | | | | |
| 21313080 | RIKEN cDNA 2700085E05 [Mus musculus] | | | | |
| 22267456 | RIKEN cDNA 2810431B21 [Mus musculus] | | | 5729820 | |
| 21312204 | RIKEN cDNA 2810435D12 [Mus musculus] | | | | |
| 19526848 | RIKEN cDNA 2810484M10 [Mus musculus] | | | | |
| 31541932 | RIKEN cDNA 2900026G05 [Mus musculus] | | | 17921985 | |
| | | | | 17921987 | |
| 21312153 | RIKEN cDNA 2900070E19 [Mus musculus] | | | | |
| 13386046 | RIKEN cDNA 3010027G13 [Mus musculus] | 13386046 | | | |
| 27229021 | RIKEN cDNA 3110001M13 [Mus musculus] | | | 4506865 | DHSD_HUMAN |
| 20822904 | RIKEN cDNA 3110004O18 [Mus musculus] | 20822904 | | 4758734 | O75439 |
| | | 25031957 | | | |
| 30424808 | RIKEN cDNA 3110021G18 [Mus musculus] | | | 15011910 | A40141 |
| 25072051 | RIKEN cDNA 3110065L21 [Mus musculus] | | | | |
| 21312006 | RIKEN cDNA 3632410G24 [Mus musculus] | 21312006 | | 4759286 | UCP4_HUMAN |
| 21311988 | RIKEN cDNA 4121402D02 [Mus musculus] | | | | |
| 13385168 | RIKEN cDNA 4430402G14 [Mus musculus] | | | | UCRI_HUMAN |
| 31981207 | RIKEN cDNA 4432405K22 [Mus musculus] | | | | |
| 19527276 | RIKEN cDNA 4921526O06 [Mus musculus] | | | | |
| 21312894 | RIKEN cDNA 4930483N21 [Mus musculus] | | | | |
| 30424611 | RIKEN cDNA 4932416F07 [Mus musculus] | | | | |
| 13386066 | RIKEN cDNA 5730591C18 [Mus musculus] | 13386066 | | 4758424 | GCHUH |
| 27370158 | RIKEN cDNA 6430520C02 [Mus musculus] | | | 5454070 | Q92581 |
| 28077029 | RIKEN cDNA 9130022B02 [Mus musculus] | | | 4758886 | S69546 |
| 13386062 | RIKEN cDNA 9430083G14 [Mus musculus] | | | | |
| 27369922 | RIKEN cDNA 9630020E24 [Mus musculus] | | | | |
| 27370474 | RIKEN cDNA 9630038C02 [Mus musculus] | | | | GABT_HUMAN |
| 22122359 | RIKEN cDNA A330009E03 [Mus musculus] | | | 5031709 | |
| 21450203 | RIKEN cDNA A330035H04; long-chain acyl-CoA | | | | |
| | synthetase [Mus musculus] | | | | |
| 21704204 | RIKEN cDNA A930031O08 [Mus musculus] | | | 4759068 | |
| 34328415 | RIKEN cDNA A930035F14 gene [Mus musculus] | | | | PUT2_HUMAN |
| 21311919 | RIKEN cDNA B430104H02 [Mus musculus] | | | | |
| 27369966 | RIKEN cDNA D530020C15 [Mus musculus] | | | 4505689 | I55465 |
| 27369748 | RIKEN cDNA D630032B01 [Mus musculus] | | | | |
| 19527384 | RIKEN cDNA D930010J01 [Mus musculus] | | | | |
| 28893421 | RIKEN cDNA E430012M05 gene [Mus musculus] | | | | |
| 22267442 | RIKubiquinol cytochrome c reductase core protein 2 | 22267442 | | | A32629 |
| | [Mus musculus] | | | | |
| 31982720 | SA rat hypertension-associated homolog [Mus | | | | |
| | musculus] | | | | |
| 20149748 | sarcosine dehydrogenase [Mus musculus] | | | | |
| 15030102 | Sdha protein [Mus musculus] | | | 4759080 | |
| 984837 | secretory group II phospholipase A2 | | | | PSHUYF |
| 6677943 | serine hydroxymethyl transferase 1 (soluble) [Mus | | | | |
| | musculus] | | | | |
| 21312298 | serine hydroxymethyl transferase 2 (mitochondrial) | | | 19923315 | B46746 |
| | [Mus musculus] | | | | |
| 15147224 | sideroflexin 1; flexed tail [Mus musculus] | 15147224 | | | |
| | | 16716499 | | | |
| 31981486 | sideroflexin 2 [Mus musculus] | 16716497 | | | |
| 16716501 | sideroflexin 4 [Mus musculus] | 16716501 | | | |
| 20895140 | similar to aminomethyltransferase [Mus musculus] | | | 4502083 | I54192 |
| 25052664 | similar to Cytochrome c oxidase assembly protein | | | 4758034 | COXZ_HUMAN |
| | COX11, mitochondrial precursor | | | | |
| 28478945 | similar to Glutaminase, kidney isoform, mitochondrial | | | 20336214 | |
| | precursor (GLS) | | | | |
| 28526374 | similar to NADH2 dehydrogenase (ubiquinone) (EC | | NUMM_MOUSE | | O75380 |
| | 1.6.5.3) complex I 13K-A chain | | | | |
| 20825073 | similar to NADH-ubiquinone oxidoreductase B17 | | | | O95139 |
| | subunit (Complex I-B17) (Cl-B17) | | | | |
| 20916351 | single-stranded DNA binding protein 1 [Mus musculus] | | | 4507231 | JN0568 |
| 27229283 | small fragment nuclease [Mus musculus] | | | | T14770 |
| 13540709 | sodium channel, voltage-gated, type 1, alpha | | | | |
| | polypeptide; sodium channel, | | | | |
| 6678001 | solute carrier family 1, member 1 [Mus musculus] | | EAT3_MOUSE | | |
| 7106409 | solute carrier family 1, member 2; glial high affinity | | | | EAT2_HUMAN |
| | glutamate transporter | | | | |
| 24233554 | solute carrier family 1, member 3; glial high affinity | | | | JC2084 |
| | glutamate transporter | | | | |
| 9790129 | solute carrier family 22 member 4; solute carrier family | | | | |
| | (organic cation | | | | |
| 28544699 | solute carrier family 25 (mitochondrial carrier), member | 20342202 | | | |
| | 18 [Mus musculus] | 20831383 | | | |
| | | 25022813 | | | |
| 6755544 | solute carrier family 25 (mitochondrial carrier, brain), | 6755544 | | 4507009 | O95258 |
| | member 14; solute | 13385736 | | 13259543 | |
| 7657583 | solute carrier family 25 (mitochondrial carrier; adenine | 7657583 | | 21361103 | Y14494 |
| | nucleotide | | | 7657581 | |
| 7305501 | solute carrier family 25 (mitochondrial carrier; | 7305501 | | | |
| | dicarboxylate transporter), | | | | |
| 6754952 | solute carrier family 25 (mitochondrial carrier; ornithine | 6754952 | | | |
| | transporter), member | | | | |
| 21312994 | solute carrier family 25 (mitochondrial carrier; | 21312994 | | | A56650 |
| | oxoglutarate carrier), member | | | | |
| 29789024 | solute carrier family 25 (mitochondrial carrier; | 20902883 | | | |
| | peroxisomal membrane protein), | | | | |
| 19526818 | solute carrier family 25 (mitochondrial carrier; | 19526818 | | 6031192 | A53737 B53737 |
| | phosphate carrier), member 3; | | | 4505775 | |
| 21313024 | solute carrier family 25 (mitochondrial deoxynucleotide | 21313024 | | | |
| | carrier), member 19 [Mus | | | | |
| 23943838 | solute carrier family 25, member 1; DiGeorge syndrome | 20346164 | | | TXTP_HUMAN |
| | gene j; solute carrier | 20891945 | | | |
| | | 23943838 | | | |
| | | 25025453 | | | |
| 22094075 | solute carrier family 25, member 5; adenine nucleotide | 20863388 | S31814 S37210 | 4502097 | A29132 A44778 |
| | translocator 2, | 22094075 | | | S03894 |
| 6755548 | solute carrier family 27 (fatty acid transporter), member | | | | |
| | 2; very long-chain | | | | |
| 31981977 | spastic paraplegia 7 homolog; paraplegin; spastic | | | 4507173 | |
| | paraplegia 7 [Mus musculus] | | | | |
| 13507712 | sphingosine-1-phosphate phosphatase 1; sphingosine- | 13507712 | | | |
| | 1-phosphate phosphatase [Mus | | | | |
| 10946984 | START domain containing 3; es64 protein; | | | | S60682 |
| | steroidogenic acute regulatory protein | | | | |
| 31543776 | steroidogenic acute regulatory protein [Mus musculus] | 19920319 | A55455 | 4507251 | I38896 |
| 28545662 | sterol carrier protein 2, liver [Mus musculus] | 20841062 | JU0157 A40015 | | B40407 |
| 12963591 | stomatin-like protein 2 [Mus musculus] | | | | |
| 13384690 | succinate dehydrogenase complex, subunit C, integral | 13384690 | | 4506863 | |
| | membrane protein [Mus | | | | |
| 20908717 | succinate dehydrogenase Fp subunit [Mus musculus] | | | | JX0336 |
| 34328286 | succinate dehydrogenase Ip subunit [Mus musculus] | | PT0094 | 9257242 | A34045 |
| 9845299 | succinate-CoA ligase, GDP-forming, alpha subunit; | 9845299 | | 11321581 | P53597 |
| | succinyl-CoA synthetase [Mus | | | | |
| 31981549 | sulfide quinone reductase-like; flavo-binding protein; | | | | |
| | sulfide | | | | |
| 30424565 | sulfite oxidase [Mus musculus] | | | | S55874 |
| 31980762 | superoxide dismutase 2, mitochondrial; manganese | 7305511 | I57023 | 10835187 | DSHUN |
| | SOD; manganese superoxide | | | | |
| 31088872 | suppressor of var1, 3-like 1 [Mus musculus] | | | 4507315 | |
| 7363455 | surfeit gene 1 [Mus musculus] | 7363455 | B25394 | | S57749 |
| 6678179 | syntaxin binding protein 1; unc18 homolog (C. elegans); | | | | |
| | UNC-18 homolog (C. | | | | |
| 15809030 | synuclein, beta [Mus musculus] | | | | |
| 31442416 | tafazzin [Mus musculus] | | | | TFZ_HUMAN |
| 13384998 | tetratricopeptide repeat domain 11 [Mus musculus] | | | | |
| 13385260 | thioesterase superfamily member 2 [Mus musculus] | | | | |
| 6755911 | thioredoxin 1; thioredoxin [Mus musculus] | | | | |
| 9903609 | thioredoxin 2; thioredoxin nuclear gene encoding | 9903609 | | | THI2_HUMAN |
| | mitochondrial protein; | | | | |
| 7305603 | thioredoxin reductase 2; human EST 573010; EST | 7305603 | | 22035672 | |
| | AA118373; TR beta [Mus musculus] | | | 22035670 | |
| | | | | 22035668 | |
| 6678449 | thiosulfate sulfurtransferase, mitochondrial [Mus | 6678449 | THTR_MOUSE | | |
| | musculus] | | | | |
| 6678357 | thymidine kinase 1 [Mus musculus] | | | | KIHUT |
| 10835111 | thymidine kinase 2, mitochondrial; thymidine kinase 2 | 10835111 | | 10281330 | |
| | [Mus musculus] | | | | |
| 6678417 | thyroid peroxidase [Mus musculus] | | | | OPHUIT |
| 6678303 | transcription factor A, mitochondrial [Mus musculus] | 6678303 | | | JC1496 |
| 26006865 | transcription termination factor, mitochondrial-like [Mus | | | 5902010 | |
| | musculus] | | | | |
| 7305573 | translocase of inner mitochondrial membrane 10 | | | | |
| | homolog [Mus musculus] | | | | |
| 7305575 | translocase of inner mitochondrial membrane 13 | | | | |
| | homolog a [Mus musculus] | | | | |
| 12025536 | translocase of inner mitochondrial membrane 23 | 12025536 | | | |
| | homolog [Mus musculus] | | | | |
| 7305577 | translocase of inner mitochondrial membrane 8 | 7305577 | | | U66035 |
| | homolog a [Mus musculus] | | | | |
| 7305579 | translocase of inner mitochondrial membrane 8 | | | | |
| | homolog b [Mus musculus] | | | | |
| 7305581 | translocase of inner mitochondrial membrane 9 | | | | |
| | homolog [Mus musculus] | | | | |
| 13324686 | translocase of outer mitochondrial membrane 20 | | | | S66619 |
| | homolog [Mus musculus] | | | | |
| 8394480 | translocase of outer mitochondrial membrane 40 | 8394480 | | | |
| | homolog; mitochondrial outer | | | | |
| 19705563 | translocator of inner mitochondrial membrane 44 [Mus | 19705563 | | | IM44_HUMAN |
| | musculus] | 25024735 | | | |
| | | 25070554 | | | |
| 33468943 | translocator of inner mitochondrial membrane a; | 25030423 | | | IM17_HUMAN |
| | translocator of inner | 20910363 | | | |
| 20270297 | trimethyllysine hydroxylase, epsilon; epsilon- | 20270297 | | | |
| | trimethyllysine 2-oxoglutarate | | | | |
| 33859692 | tRNA nucleotidyl transferase, CCA-adding, 1; tRNA | 20829254 | | | |
| | adenylyltransferase, | | | | |
| 16716569 | trypsinogen 16 [Mus musculus] | | | | |
| 31543952 | tryptophanyl tRNA synthetase 2 (mitochondrial) [Mus | 21362271 | | 7710154 | |
| | musculus] | | | | |
| 6678469 | tubulin, alpha 6; tubulin alpha 6 [Mus musculus] | | | | |
| 12963615 | tubulin, beta 3 [Mus musculus] | | | | |
| 31981925 | tyrosine 3-monooxygenase/tryptophan 5- | | | | 143E_HUMAN |
| | monooxygenase activation protein, epsilon | | | | |
| 6756041 | tyrosine 3-monooxygenase/tryptophan 5- | | JC5384 | | PSHUAM |
| | monooxygenase activation protein, zeta | | | | |
| 22122769 | tyrosine aminotransferase [Mus musculus] | | | | S10887 |
| 21539599 | ubiquinol cytochrome c reductase hinge protein; | 21539599 | | | S00219 |
| | mitochondrial hinge protein; | | | | |
| 13385726 | ubiquinol-cytochrome c reductase binding protein [Mus | 13385726 | | | A32450 |
| | musculus] | | | | |
| 13384794 | ubiquinol-cytochrome c reductase core protein 1 [Mus | 13384794 | | | A48043 |
| | musculus] | 25030421 | | | |
| 13385112 | ubiquinol-cytochrome c reductase subunit [Mus | | | | |
| | musculus] | | | | |
| 21070950 | ubiquitin C; polyubiquitin C [Mus musculus] | | | | |
| 6678497 | uncoupling protein 1, mitochondrial; uncoupling protein, | 6678497 | A31106 | 11225256 | A60793 |
| | mitochondrial [Mus | | | | |
| 31543920 | uncoupling protein 2, mitochondrial [Mus musculus] | 6755933 | | | UCP2_HUMAN |
| 6678495 | uncoupling protein 3, mitochondrial [Mus musculus] | 6678495 | | | |
| 12836291 | unnamed protein product [Mus musculus] | | | 21396489 | S42366 |
| 12832533 | unnamed protein product [Mus musculus] | | | | O75489 |
| 12832556 | unnamed protein product [Mus musculus] | | | | O96000 |
| 26343407 | unnamed protein product [Mus musculus] | | | 14790138 | |
| 26346947 | unnamed protein product [Mus musculus] | | | | S63453 |
| 12834221 | unnamed protein product [Mus musculus] | | | | |
| 12834781 | unnamed protein product [Mus musculus] | | | | |
| 12835668 | unnamed protein product [Mus musculus] | | | | |
| 12835711 | unnamed protein product [Mus musculus] | | | | |
| 12836533 | unnamed protein product [Mus musculus] | | | | |
| 12836798 | unnamed protein product [Mus musculus] | | | | |
| 12841269 | unnamed protein product [Mus musculus] | | | | |
| 12842244 | unnamed protein product [Mus musculus] | | | | |
| 12845262 | unnamed protein product [Mus musculus] | | | | |
| 12846164 | unnamed protein product [Mus musculus] | | | | |
| 12855263 | unnamed protein product [Mus musculus] | | | | |
| 12855887 | unnamed protein product [Mus musculus] | | | | |
| 12860092 | unnamed protein product [Mus musculus] | | | | |
| 12861374 | unnamed protein product [Mus musculus] | | | | |
| 26363071 | unnamed protein product [Mus musculus] | | | | |
| 13128954 | upregulated during skeletal muscle growth 5 [Mus | | | | |
| | musculus] | | | | |
| 6755941 | uracil-DNA glycosylase [Mus musculus] | 6755941 | UNG_MOUSE | | A60472 |
| 6678509 | urate oxidase; uricase [Mus musculus] | | | | |
| 6678519 | uroporphyrinogen III synthase; URO-synthase; | | | | A40483 |
| | uroporphyrinogen-III synthase; | | | | |
| 34328204 | valyl-tRNA synthetase 2 [Mus musculus] | | | | |
| 31559883 | very-long-chain acyl-CoA dehydrogenase VLCAD | | | | |
| | homolog [Mus musculus] | | | | |
| 6755963 | voltage-dependent anion channel 1 [Mus musculus] | 6755963 | | 4507879 | MMHUP3 |
| 6755965 | voltage-dependent anion channel 2 [Mus musculus] | 6755965 | | | B44422 |
| 6755967 | voltage-dependent anion channel 3 [Mus musculus] | | | | S59547 |
| 31980962 | WW-domain oxidoreductase [Mus musculus] | 9625012 | | | |

TABLE 5
Tiers of evidence supporting the 163 newly identified mito-A proteins. The protein accession and description of each of the newly
identified mito-A proteins is shown along with each of the GenPept accessions of the proteins identified in the tissue proteomics experiments.
For each mito-A protein cluster, the top scoring human homologue from the study, the PSORT targeting prediction, the mitochondrial
neighborhood index, and the results of epitope tagging experiments, when available, are shown. For the BLASTP analyses, only the top scoring
match from the study by MitoKor is provided, using a threshold of E < 1 × 10⁻⁵. The PSORT targeting prediction and probability were obtained for
the exemplar protein sequence. The neighborhood indices (N₅₀, N₁₀₀, and N₂₅₀) are provided, when available. Due to probe-set duplicity, some
proteins have more than one corresponding probe-set, and others have no probe-set. An N₅₀ ≧ 6, N₁₀₀ ≧ 10, and N₂₅₀ ≧ 19 each correspond to a
nominal P = 0.001, assuming that mito-A genes are randomly distributed in expression space. In the final column, the subcellular localization
based on immunofluorescence microscopy is indicated for the five proteins shown in FIG. 2

| Exemplar Protein for the Cluster | | Proteomics | | | | BLASTP against MitoKor | | |
|---|---|---|---|---|---|---|---|---|
| Accession | Description | Liver | Brain | Heart | Kidney | Match | Score | Expect |
| 21313679 | RIKEN cDNA 0610009D10 [Mus musculus] | 12832313 | 12832313 | 12832313 | 12832313 | 5453559 | 283 | 1.00E−78 |
| | | 220904 | | | 220904 | | | |
| 21312594 | RIKEN cDNA 2610205H19; EST AA108335 | 12848292 | 12848292 | 12848292 | 730248 | 7661602 | 249 | 2.00E−68 |
| | [Mus musculus] | 730248 | | 730248 | | | | |
| 13128954 | upregulated during skeletal muscle growth 5 [Mus | 12842476 | 13128954 | 12842476 | 12842476 | 14249376 | 105 | 2.00E−25 |
| | musculus] | 6851054 | 12842476 | | | | | |
| 6671622 | B-cell receptor-associated protein 37; repressor of | 6005854 | 6005854 | 6005854 | 6005854 | 6005854 | 568 | e−164 |
| | estrogen receptor activity | 6671622 | | | | | | |
| 27228985 | RIKEN cDNA 2410011G03 [Mus musculus] | 10092657 | 13384978 | 13384978 | 13384978 | 10092657 | 297 | 6.00E−83 |
| | | 13384978 | | | | | | |
| 13384766 | RIKEN cDNA 1110021D01 [Mus musculus] | 13384766 | 13384766 | 12842709 | 13384766 | NO | | |
| | | 12842709 | | | | MATCH | | |
| 19354491 | 1110020P15Rik protein [Mus musculus] | 136701 | 9297078 | 136701 | 136701 | 9297078 | 116 | 5.00E−29 |
| | | | 136701 | | | | | |
| | | | 3891857 | | | | | |
| | | | 6094658 | | | | | |
| 9789997 | leucine zipper-EF-hand containing transmembrane | 9789997 | 9789997 | 9789997 | 9789997 | 6912482 | 1209 | 0 |
| | protein 1; leucine | | | | | | | |
| 13385260 | thioesterase superfamily member 2 [Mus musculus] | 13385260 | 13385260 | 13385260 | 13385260 | 4210351 | 209 | 2.00E−56 |
| 19527228 | DNA segment, Chr 10, ERATO Doi 214, expressed | 8923930 | 8923930 | 8923930 | 8923930 | 8923930 | 206 | 1.00E−55 |
| | [Mus musculus] | | | | | | | |
| 12842244 | unnamed protein product [Mus musculus] | 12842244 | 12842244 | 12842244 | 12842244 | 17455445 | 210 | 1.00E−56 |
| 12963633 | genes associated with retinoid-IFN-induced | 12963633 | 12963633 | 12963633 | 12963633 | 12005918 | 260 | 1.00E−71 |
| | mortality 19 [Mus musculus] | 12833386 | 12833406 | 12833386 | 12833386 | | | |
| | | 12833406 | | 12833406 | 7705734 | | | |
| | | | | | 12833406 | | | |
| 6679066 | 4-nitrophenylphosphatase domain and non- | 6679066 | 4505399 | 6679066 | 12803135 | 4503937 | 429 | e−122 |
| | neuronal SNAP25-like protein homolog 1 | 12850319 | 6679066 | | 4505399 | | | |
| | | | 12850319 | | 6679066 | | | |
| | | | | | 12850319 | | | |
| 7949047 | hydroxyacyl-Coenzyme A dehydrogenase type II; | 7949047 | 7949047 | 7949047 | 7949047 | 14764202 | 421 | e−120 |
| | hydroxyacyl-Coenzyme A | 12850643 | | 12850643 | 13182962 | | | |
| | | 13182962 | | 13182962 | | | | |
| | | 3183025 | | | | | | |
| 23956104 | adenylate kinase 3 alpha-like; adenylate kinase 3 | 12837588 | | 12837588 | 12836369 | 12735226 | 428 | e−122 |
| | alpha like [Mus musculus] | 6978479 | | | 12837588 | | | |
| | | 6707707 | | | 6707707 | | | |
| 20149748 | sarcosine dehydrogenase [Mus musculus] | 13097441 | | 13097441 | 13097441 | 13775158 | 185 | 3.00E−48 |
| | | 3283373 | | | 3283373 | | | |
| | | 4928113 | | | | | | |
| 31980804 | peroxisomal trans 2-enoyl CoA reductase; | 12963715 | | 12963715 | 12845570 | 4503301 | 143 | 5.00E−36 |
| | perosisomal 2-enoyl-CoA reductase [Mus | 13506791 | | | 12963715 | | | |
| | | | | | 13506791 | | | |
| 21624609 | RIKEN cDNA 2010012D11 [Mus musculus] | 12833236 | | 12857234 | 12833236 | NO | | |
| | | 12857234 | | | 4757862 | MATCH | | |
| 21389320 | leucine-rich PPR motif-containing protein; leucine | 12851540 | | 1730078 | 12851540 | 1730078 | 1938 | 0 |
| | rich protein LRP130 [Mus | | | 12851540 | | | | |
| 21313618 | RIKEN cDNA 0610041L09 [Mus musculus] | | 12839842 | 12832121 | 12832121 | 8923390 | 411 | e−117 |
| | | | | 8923390 | | | | |
| 30424611 | RIKEN cDNA 4932416F07 [Mus musculus] | 7513021 | | 7513021 | 7513021 | NO | | |
| | | | | | | MATCH | | |
| 27369748 | RIKEN cDNA D630032B01 [Mus musculus] | 1711535 | 1711535 | | 1711535 | 13630862 | 608 | e−176 |
| 34328379 | D-lactate dehydrogenase [Mus musculus] | 12852638 | | 12852638 | 12852638 | NO | | |
| | | | | | | MATCH | | |
| 19526848 | RIKEN cDNA 2810484M10 [Mus musculus] | 3747107 | 3747107 | | 3747107 | NO | | |
| | | | | | | MATCH | | |
| 19482166 | kidney expressed gene 1 [Mus musculus] | 12832283 | | 12832283 | 12832283 | NO | | |
| | | | | | | MATCH | | |
| 6754092 | glutathione transferase zeta 1 (maleylacetoacetate | 6754092 | | 6754092 | 6754092 | NO | | |
| | isomerase); | | | | | MATCH | | |
| 21312153 | RIKEN cDNA 2900070E19[Mus musculus] | 12851249 | 12851249 | | 12851249 | 12735430 | 101 | 6.00E−24 |
| 13384742 | RIKEN cDNA 1110018B13 [Mus musculus] | | 13384742 | 13384742 | 13384742 | 15150811 | 175 | 2.00E−46 |
| 12835711 | unnamed protein product[Mus musculus] | 12835711 | | 12835711 | 12835711 | 14211923 | 290 | 1.00E−80 |
| 13507612 | NADPH-dependent retinol | 13097510 | | 13507612 | 11559414 | 12804319 | 51 | 1.00E−08 |
| | dehydrogenase/reductase [Mus musculus] | | | 12832859 | | | | |
| 34328185 | prosaposin [Mus musculus] | 7242191 | 6981424 | | 91281 | NO | | |
| | | 91281 | 881390 | | 881390 | MATCH | | |
| | | 557967 | | | | | | |
| | | 6981424 | | | | | | |
| | | 881390 | | | | | | |
| | | 9438805 | | | | | | |
| | | 1360694 | | | | | | |
| | | 11386147 | | | | | | |
| 13540709 | sodium channel, voltage-gated, type 1, alpha | 13540709 | | 13540709 | | NO | | |
| | polypeptide; sodium channel, | | | | | MATCH | | |
| 21070950 | ubiquitin C; polyubiquitin C [Mus musculus] | 9790277 | 9790277 | | | 11024714 | 449 | e−128 |
| | | | 1050930 | | | | | |
| | | | 136670 | | | | | |
| 31980703 | aminoadipate-semialdehyde synthase; lysine | 13529344 | | | 13027640 | NO | | |
| | oxoglutarate reductase, saccharopine | 8393730 | | | 13529344 | MATCH | | |
| | | | | | 4938304 | | | |
| | | | | | 8393730 | | | |
| 6753272 | catalase; catalase 1 [Mus musculus] | 6753272 | | | 115704 | NO | | |
| | | | | | 6753272 | MATCH | | |
| | | | | | 115698 | | | |
| | | | | | 229299 | | | |
| 31541815 | L-specific multifunctional beta-oxdiation protein | 12836375 | | | 1706569 | 14730775 | 293 | 9.00E−81 |
| | [Mus musculus] | | | | 11434714 | | | |
| | | | | | 12836375 | | | |
| 7656855 | acyl-Coenzyme A oxidase 1, palmitoyl; acyl- | 6429156 | | | 6429156 | 13653049 | 55 | 3.00E−09 |
| | Coenzyme A oxidase; Acyl-CoA oxidase | 7656855 | | | 7656855 | | | |
| 9790129 | solute carrier family 22 member 4; solute carrier | 9790129 | 9790129 | | | NO | | |
| | family (organic cation | | | | | MATCH | | |
| 6680756 | ATPase, H+ transporting, V1 subunit E isoform 1; | | 6680756 | | 6680756 | NO | | |
| | ATPase, H+ transporting | | 313014 | | | MATCH | | |
| 201006 | Cu/Zn-superoxide dismutase | 201006 | | | 134614 | 1237406 | 266 | 2.00E−73 |
| | | | | | 1351080 | | | |
| | | | | | 226471 | | | |
| | | | | | 7433299 | | | |
| 9055178 | brain protein 44-like; apoptosis-regulating basic | 12852262 | 12852262 | | | 14755192 | 216 | 1.00E−58 |
| | protein [Mus musculus] | 7706369 | 12852283 | | | | | |
| | | 9055178 | | | | | | |
| 7305125 | estradiol 17 beta-dehydrogenase 8; 17-beta- | 7305125 | | | 1103844 | 14041699 | 418 | e−119 |
| | hydroxysteroid dehydrogenase 8; | 1103844 | | | | | | |
| 12963539 | HSCO protein [Mus musculus] | 12832819 | | | 12963539 | 4885389 | 70 | 3.00E−14 |
| | | | | | 12832819 | | | |
| 21312020 | hypothetical protein D4Ertd765e [Mus musculus] | 12836667 | | | 12836667 | 4502327 | 300 | 2.00E−83 |
| | | | | | 12847441 | | | |
| 12963697 | RIKEN cDNA 1110025H10 [Mus musculus] | 12963697 | | | 12963697 | NO | | |
| | | | | | 12834868 | MATCH | | |
| 6681137 | diazepam binding inhibitor; acyl-CoA binding | 13937379 | | | 13937379 | 12052810 | 76 | 1.00E−16 |
| | protein; diazepam-binding inhibitor | 6681137 | | | | | | |
| 13507620 | ankycorbin; NORPEG-like protein [Mus musculus] | | 13507620 | 13507620 | | 14771689 | 100 | 2.00E−22 |
| 16905127 | butyryl Coenzyme A synthetase 1; acetyl- | 5019275 | | | 15487300 | 6996429 | 137 | 6.00E−34 |
| | Coenzyme A synthetase 3 [Mus musculus] | | | | | | | |
| 22122743 | hypothetical protein MGC37245 [Mus musculus] | | | 3127193 | 3127193 | 6996429 | 123 | 7.00E−30 |
| 22203753 | inorganic pyrophosphatase 2 [Mus musculus] | 12834464 | | | 12834464 | 11526789 | 525 | e−151 |
| 13385656 | RIKEN cDNA 0610010D20 [Mus musculus] | 13385656 | | | 13385656 | NO | | |
| | | 12846589 | | | | MATCH | | |
| 33859690 | RIKEN cDNA 2310005O14 [Mus musculus] | 3252827 | | 3252827 | | 3252827 | 578 | e−167 |
| 21311919 | RIKEN cDNA B430104H02 [Mus musculus] | 7705608 | | | 12836847 | NO | | |
| | | | | | | MATCH | | |
| 21703764 | lactamase, beta 2 [Mus musculus] | 13278495 | | | 13278495 | NO | | |
| | | | | | | MATCH | | |
| 13385662 | RIKEN cDNA 0610042E07 [Mus musculus] | 13376007 | | | 13376007 | NO | | |
| | | | | | | MATCH | | |
| 10946936 | adenylate kinase 1; cytosolic adenylate kinase [Mus | | | 729865 | 125152 | 4502011 | 347 | 6.00E−98 |
| | musculus] | | | | | | | |
| 6680277 | heat-responsive protein 12 [Mus musculus] | 6680277 | | | 6680277 | 5032215 | 226 | 3.00E−61 |
| 21312028 | RIKEN cDNA 1110006I11 [Mus musculus] | 12834206 | | | 12834206 | NO | | |
| | | | | | | MATCH | | |
| 13385436 | RIKEN cDNA 2010100O12 [Mus musculus] | 13385436 | | | 13385436 | NO | | |
| | | | | | | MATCH | | |
| 12836533 | unnamed protein product [Mus musculus] | | | 12836533 | 12836533 | NO | | |
| | | | | | | MATCH | | |
| 6677943 | serine hydroxymethyl transferase 1 (soluble) [Mus | 232178 | 232178 | | | NO | | |
| | musculus] | | | | | MATCH | | |
| 12834221 | unnamed protein product [Mus musculus] | 12834221 | | | 12834221 | 14211939 | 283 | 1.00E−78 |
| 6681097 | cytochrome P450, family 17, subfamily a, | 2148066 | | 2506241 | | NO | | |
| | polypeptide 1; cytochrome P450, 17; | | | | | MATCH | | |
| 6753676 | dihydropyrimidinase-like 2; collapsin response | | 1351260 | | | 13645618 | 825 | 0 |
| | mediator protein 2 [Mus musculus] | | 3122018 | | | | | |
| 79937 | glyceraldehyde-3-phosphate dehydrogenase [Mus | 6679937 | | | | 7669492 | 637 | 0 |
| | musculus] | 229279 | | | | | | |
| | | 65987 | | | | | | |
| | | 9838358 | | | | | | |
| 13435924 | aldolase 3, C isoform [Mus musculus] | | 11231095 | | | 312137 | 716 | 0 |
| | | | 12836758 | | | | | |
| 31982332 | glutamate-ammonia ligase (glutamine synthase); | | 2144562 | | | NO | | |
| | glutamine synthetase [Mus | | 4504027 | | | MATCH | | |
| | | | 6680023 | | | | | |
| | | | 2144563 | | | | | |
| 6681079 | cathepsin B preproprotein [Mus musculus] | 227293 | | | | NO | | |
| | | 6681079 | | | | MATCH | | |
| | | 12832453 | | | | | | |
| | | 3929817 | | | | | | |
| 13654245 | major urinary protein 1 [Mus musculus] | 13276755 | | | | NO | | |
| | | 127531 | | | | MATCH | | |
| 27369922 | RIKEN cDNA 9630020E24 [Mus musculus] | | 12052944 | | | 7513022 | 108 | 4.00E−25 |
| 6680305 | heat shock protein 1, beta; heat shock protein, 84 kDa | | 1170383 | | | 72222 | 1415 | 0 |
| | 1; heat shock 90 kDa | | 3642691 | | | | | |
| 31982847 | glutamic acid decarboxylase 1 [Mus musculus] | 416884 | | | | NO | | |
| | | 1082397 | | | | MATCH | | |
| | | 1352214 | | | | | | |
| 31981147 | leucine aminopeptidase 3; leucine aminopeptidase | 12845995 | | | | NO | | |
| | [Mus musculus] | 7705688 | | | | MATCH | | |
| | | 12833083 | | | | | | |
| 6753556 | cathepsin D [Mus musculus] | 6753556 | | | | 4503143 | 697 | 0 |
| | | 115720 | | | | | | |
| | | 8886526 | | | | | | |
| 31560731 | ATPase, H+ transporting, V1 subunit A, isoform 1; | | 108733 | | | 114549 | 116 | 1.00E−27 |
| | ATPase, H+ transporting, | | 6680752 | | | | | |
| 6680107 | granulin; acrogranulin; progranulin; PC cell-derived | 191767 | | | | 1335064 | 57 | 8.00E−10 |
| | growth factor [Mus | 6680107 | | | | | | |
| 31982720 | SA rat hypertension-associated homolog [Mus | | | | 2135243 | 6996429 | 161 | 2.00E−41 |
| | musculus] | | | | 5032065 | | | |
| 6753448 | ceroid-lipofuscinosis, neuronal 2 [Mus musculus] | 13786206 | | | | NO | | |
| | | 6753448 | | | | MATCH | | |
| 6754408 | kynurenine aminotransferase II [Mus musculus] | | | | 6754408 | NO | | |
| | | | | | 8393641 | MATCH | | |
| 14780884 | polymerase delta interacting protein 38 [Mus | 7661672 | | | | NO | | |
| | musculus] | 12834531 | | | | MATCH | | |
| 31543280 | putative prostate cancer tumor suppressor; cDNA | | | | 1353701 | NO | | |
| | sequence BC003311 [Mus musculus] | | | | | MATCH | | |
| 12963555 | Nit protein 2 [Mus musculus] | | | | 12963555 | NO | | |
| | | | | | 12835765 | MATCH | | |
| 27754146 | RIKEN cDNA 0710001P09 [Mus musculus] | | 12853604 | | | 14150134 | 301 | 8.00E−84 |
| | | | 12839157 | | | | | |
| 27754071 | hypothetical protein 4833421E05Rik [Mus | | | | 12837739 | NO | | |
| | musculus] | | | | 12847330 | MATCH | | |
| 31981013 | methionine sulfoxide reductase A [Mus musculus] | | | | 12844852 | NO | | |
| | | | | | 12857997 | MATCH | | |
| 13384998 | tetratricopeptide repeat domain 11 [Mus musculus] | | | | 13384998 | 14747249 | 288 | 4.00E−80 |
| | | | | | 7705632 | | | |
| 9506933 | neuronal protein 15.6 [Mus musculus] | | | 9506933 | | 13938442 | 220 | 8.00E−60 |
| 21311867 | hypothetical protein D11Ertd99e [Mus musculus] | | | | 12859025 | 7661732 | 174 | 4.00E−46 |
| | | | | | 7661732 | | | |
| 6678716 | low density lipoprotein receptor-related protein 5; | | | 7513560 | | 1335064 | 53 | 3.00E−08 |
| | low density | | | | | | | |
| 34328204 | valyl-tRNA synthetase 2 [Mus musculus] | | | | 6755953 | 7678804 | 191 | 5.00E−50 |
| 30794396 | RIKEN cDNA 2410021P16 [Mus musculus] | | | | 12846107 | 13653049 | 141 | 6.00E−35 |
| 31982273 | hydroxysteroid (17-beta) dehydrogenase 4; | | | | 12836373 | 14041699 | 100 | 1.00E−22 |
| | hydroxysteroid 17-beta dehydrogenase | | | | | | | |
| 21450203 | RIKEN cDNA A330035H04; long-chain acyl-CoA | 4336604 | | | | 11276083 | 981 | 0 |
| | synthetase [Mus musculus] | | | | | | | |
| 31981207 | RIKEN cDNA 4432405K22 [Mus musculus] | | | 12232451 | | NO | | |
| | | | | | | MATCH | | |
| 6680612 | ATP-binding cassette, sub-family D, member 3; | | | | 105161 | NO | | |
| | peroxisomal membrane protein, 70 | | | | | MATCH | | |
| 31559883 | very-long-chain acyl-CoA dehydrogenase VLCAD | | 12849737 | | | 10436258 | 1056 | 0 |
| | homolog [Mus musculus] | | | | | | | |
| 6755548 | solute carrier family 27 (fatty acid transporter), | | | | 3087820 | 15559516 | 61 | 4.00E−11 |
| | member 2; very long-chain | | | | | | | |
| 21311988 | RIKEN cDNA 4121402D02 [Mus musculus] | | | | 12853862 | NO | | |
| | | | | | | MATCH | | |
| 6678179 | syntaxin binding protein 1; unc18 homolog (C. elegans); | | 6981602 | | | NO | | |
| | UNC-18 homolog (C. | | | | | MATCH | | |
| 30725845 | AAA-ATPase TOB3 [Mus musculus] | | | | 13752413 | 11095436 | 57 | 8.00E−10 |
| 31981562 | pyruvate kinase 3 [Mus musculus] | | 6755074 | | | 107554 | 1032 | 0 |
| 11968160 | 3-oxoacid CoA transferase 2A; haploid germ cell | | | | 11968160 | 4557817 | 709 | 0 |
| | specific succinyl CoA | | | | | | | |
| 20070418 | aldehyde dehydrogenase family 7, member A1; | 12836597 | | | | 12803387 | 953 | 0 |
| | aldehyde dehydrogenase 7 family, | | | | | | | |
| 13195670 | RIKEN cDNA 2610207I16 [Mus musculus] | 13195670 | | | | 14150062 | 374 | e−105 |
| 19527030 | kynurenine 3-monooxygenase (kynurenine 3- | 11024672 | | | | NO | | |
| | hydroxylase) [Mus musculus] | | | | | MATCH | | |
| 6679437 | protective protein for beta-galactosidase [Mus | | | | 12860234 | NO | | |
| | musculus] | | | | | MATCH | | |
| 31981549 | sulfide quinone reductase-like; flavo-binding | | | | 12842384 | 10864011 | 812 | 0 |
| | protein; sulfide | | | | | | | |
| 6753074 | adaptor protein complex AP-2, mu1; adaptor- | | 6753074 | | | NO | | |
| | related protein complex AP-2, mu1; | | | | | MATCH | | |
| 28893421 | RIKEN cDNA E430012M05 gene [Mus musculus] | | 12654733 | | | NO | | |
| | | | | | | MATCH | | |
| 19527276 | RIKEN cDNA 4921526O06 [Mus musculus] | | | | 7705586 | NO | | |
| | | | | | | MATCH | | |
| 27659728 | aldo-keto reductase family 7, member A5 (aflatoxin | 13384704 | | | | NO | | |
| | aldehyde reductase); | | | | | MATCH | | |
| 14861848 | DNA segment, Chr 7, Roswell Park 2 complex, | | | | 14861848 | NO | | |
| | expressed; androgen regulated gene | | | | | MATCH | | |
| 12963591 | stomatin-like protein 2 [Mus musculus] | 12963591 | | | | 7513076 | 603 | e−174 |
| 6753058 | annexin A10 [Mus musculus] | | 6274497 | | | 4826643 | 271 | 1.00E−74 |
| 12834781 | unnamed protein product [Mus musculus] | 12834781 | | | | NO | | |
| | | 12856019 | | | | MATCH | | |
| 18875408 | peroxisomal acyl-CoA thioesterase 1 [Mus | | | | 4885565 | NO | | |
| | musculus] | | | | | MATCH | | |
| 11968166 | cathepsin Z preproprotein; cathepsin Z precursor; | 12835144 | | | | NO | | |
| | cathepsin X [Mus musculus] | | | | | MATCH | | |
| 31560255 | RIKEN cDNA 2410005O16 [Mus musculus] | 13384896 | | | | 16307164 | 511 | e−147 |
| 6678509 | urate oxidase; uricase [Mus musculus] | 6678509 | | | | NO | | |
| | | | | | | MATCH | | |
| 31980955 | RIKEN cDNA 2310005D12 [Mus musculus] | 13195640 | | | | 12654521 | 474 | e−136 |
| 21313080 | RIKEN cDNA 2700085E05 [Mus musculus] | 12840992 | | | | NO | | |
| | | | | | | MATCH | | |
| 6755334 | ribonuclease H1 [Mus musculus] | | | | 3004981 | NO | | |
| | | | | | | MATCH | | |
| 6679957 | glioblastoma amplified [Mus musculus] | | | 6679957 | | 4503937 | 540 | e−156 |
| 7948999 | peroxiredoxin 4; antioxidant enzyme AOE372; Prx | 12407849 | | | | 14768743 | 464 | e−133 |
| | IV [Mus musculus] | | | | | | | |
| 13386062 | RIKEN cDNA 9430083G14 [Mus musculus] | | | | 13386062 | 17461670 | 414 | e−118 |
| 21311883 | RIKEN cDNA 0610007O07 [Mus musculus] | 12858578 | | | | NO | | |
| | | | | | | MATCH | | |
| 21312204 | RIKEN cDNA 2810435D12 [Mus musculus] | 12850490 | | | | 13654294 | 400 | e−113 |
| 13385084 | NIPSNAP-related protein [Mus musculus] | 13385084 | | | | 14743031 | 416 | e−118 |
| 19527384 | RIKEN cDNA D930010J01 [Mus musculus] | | | 12653017 | | 12653017 | 458 | e−131 |
| 6678760 | lysophospholipase 1; phospholipase 1a; | | | | 6678760 | 14747375 | 249 | 3.00E−68 |
| | lysophopholipase 1 [Mus musculus] | | | | | | | |
| 21312894 | RIKEN cDNA 4930483N21 [Mus musculus] | | 12854111 | | | 8922629 | 56 | 5.00E−10 |
| 21313138 | glutathione S-transferase class kappa [Mus | 12832811 | | | | 7705704 | 350 | 1.00E−98 |
| | musculus] | | | | | | | |
| 21311853 | RIKEN cDNA 0610012H03 [Mus musculus] | | | | 12832709 | NO | | |
| | | | | | | MATCH | | |
| 21311967 | RIKEN cDNA 0610008C08 [Mus musculus] | | | | 12832215 | 12001992 | 287 | 1.00E−79 |
| 13384950 | RIKEN cDNA 2310039H17 [Mus musculus] | | | 13384950 | | NO | | |
| | | | | | | MATCH | | |
| 12746414 | growth factor, erv1 (S. cerevisiae)-like (augmenter | 7670387 | | | | NO | | |
| | of liver regeneration); | | | | | MATCH | | |
| 6806917 | GM2 ganglioside activator protein [Mus musculus] | | | | 479912 | NO | | |
| | | | | | | MATCH | | |
| 7305137 | heme binding protein 1; heme-binding protein; p22 | | | | 4886904 | NO | | |
| | HBP; heme-binding protein 1 | | | | | MATCH | | |
| 25092662 | DNA segment, Chr 11, Wayne State University 68, | | | | 13386160 | NO | | |
| | expressed [Mus musculus] | | | | | MATCH | | |
| 21313484 | nitrogen fixation cluster-like [Mus musculus] | | | | 12843563 | NO | | |
| | | | | | | MATCH | | |
| 18079334 | ethanol induced 6 [Mus musculus] | | | | 12834045 | NO | | |
| | | | | | | MATCH | | |
| 6679078 | expressed in non-metastatic cells 2, protein; | | 13929192 | | | 1421609 | 311 | 5.00E−87 |
| | expressed in non-metastatic cells | | | | | | | |
| 13385042 | RIKEN cDNA 2010309E21 [Mus musculus] | 13385042 | | | | NO | | |
| | | | | | | MATCH | | |
| 15809030 | synuclein, beta [Mus musculus] | | 464424 | | | NO | | |
| | | | | | | MATCH | | |
| 6755911 | thioredoxin 1; thioredoxin [Mus musculus] | | | | 12841560 | 14740403 | 196 | 1.00E−52 |
| 20841184 | acetyl-Coenzyme A carboxylase beta [Mus | | | 3080546 | | NO | | |
| | musculus] | | | | | MATCH | | |
| 25072051 | RIKEN cDNA 3110065L21 [Mus musculus] | | 4758012 | | | NO | | |
| | | | | | | MATCH | | |
| 20071710 | 2010002H18Rik protein [Mus musculus] | | | 7678804 | | 7678804 | 954 | 0 |
| 200022 | neurofilament protein | | | | 205686 | 14742600 | 301 | 4.00E−83 |
| 21618729 | Facl5 protein [Mus musculus] | | | | 10800088 | 7706449 | 1193 | 0 |
| 17505907 | DEAD (Asp-Glu-Ala-Asp) box polypeptide 31 | | | 12232467 | | NO | | |
| | isoform 1; DEAD/DEXH helicase DDX31 | | | | | MATCH | | |
| 12855263 | unnamed protein product [Mus musculus] | | | | 12855263 | NO | | |
| | | | | | | MATCH | | |
| 12855887 | unnamed protein product [Mus musculus] | 12855887 | | | | NO | | |
| | | | | | | MATCH | | |
| 26363071 | unnamed protein product [Mus musculus] | | 12843537 | | | 14771689 | 107 | 4.00E−25 |
| 12836798 | unnamed protein product [Mus musculus] | | | 12836798 | | NO | | |
| | | | | | | MATCH | | |
| 12846164 | unnamed protein product [Mus musculus] | 12846164 | | | | NO | | |
| | | | | | | MATCH | | |
| 12845262 | unnamed protein product [Mus musculus] | | | | 12845262 | 14770968 | 326 | 3.00E−91 |
| 12860092 | unnamed protein product [Mus musculus] | 12860092 | | | | 11545863 | 316 | 2.00E−88 |
| 20897872 | RIKEN cDNA 1810058I14 [Mus musculus] | | | | 12841742 | NO | | |
| | | | | | | MATCH | | |
| 12841269 | unnamed protein product [Mus musculus] | 12841269 | | | | NO | | |
| | | | | | | MATCH | | |
| 12835668 | unnamed protein product [Mus musculus] | 12835668 | | | | NO | | |
| | | | | | | MATCH | | |
| 12861374 | unnamed protein product [Mus musculus] | | | | 12861374 | NO | | |
| | | | | | | MATCH | | |
| 22267464 | retinoic acid inducible protein 3 [Mus musculus] | | | | 13436248 | NO | | |
| | | | | | | MATCH | | |

TABLE 6
The ordered gene list for FIGS. 7 and 8. The list is ordered based on FIGS. 7 and 8, and each row includes the corresponding
Affymetrix probe-set ID, protein accession, the gene symbol, evidence (white, previously annotated; gray, detected in
proteomics; black, previously annotated and detected in proteomics), the module annotation, and the description

| | | Protein | | |
|---|---|---|---|---|
| Row | Probe Set | Exemplar | Description | Symbol |
| 1 | 104560_at | 21553115 | putative mitochondrial solute carrier [Mus musculus] | Mrs3/4-pending |
| 2 | 97868_at | 31560085 | DnaJ (Hsp40) homolog, subfamily A, member 3 [Mus musculus] | Dnaja3 |
| 3 | 95608_at | 6681079 | cathepsin B preproprotein [Mus musculus] | Ctsb |
| 4 | 95359_at | 6680305 | heat shock protein 1, beta; heat shock protein, 84 kDa 1; heat shock 90 kDa | Hspcb |
| 5 | 104103_at | 30725845 | AAA-ATPase TOB3 [Mus musculus] | TOB3 |
| 6 | 96861_at | 30519921 | mitochondrial ribosomal protein L50 [Mus musculus] | D4Wsu125e |
| 7 | 95438_at | 31559891 | mitochondrial Rho 1 [Mus musculus] | 2210403N23Rik |
| 8 | 95431_at | 27552760 | DNA segment, Chr 16, Indiana University Medical 22, expressed [Mus | D16lum22e |
| | | | musculus] | |
| 9 | 93808_at | 6671688 | carbonyl reductase 2; lung carbonyl reductase [Mus musculus] | Cbr2 |
| 10 | 103044_g_at | 6754760 | mature T-cell proliferation 1 [Mus musculus] | Mtcp1 |
| 11 | 104747_at | 6678001 | solute carrier family 1, member 1 [Mus musculus] | Slc1a1 |
| 12 | 104748_s_at | 6678001 | solute carrier family 1, member 1 [Mus musculus] | Slc1a1 |
| 13 | 104700_at | 6677943 | serine hydroxymethyl transferase 1 (soluble) [Mus musculus] | Shmt1 |
| 14 | 98470_at | 6755544 | solute carrier family 25 (mitochondrial carrier, brain), member 14; solute | Slc25a14 |
| 15 | 97935_at | 21311988 | RIKEN cDNA 4121402D02 [Mus musculus] | — |
| 16 | 103061_at | 31982847 | glutamic acid decarboxylase 1 [Mus musculus] | Gad1 |
| 17 | 95432_f_at | 27552760 | DNA segment, Chr 16, Indiana University Medical 22, expressed [Mus | D16lum22e |
| | | | musculus] | |
| 18 | 95746_at | 31560731 | ATPase, H+ transporting, V1 subunit A, isoform 1; ATPase, H+ transporting, | B230379M23Rik |
| 19 | 93126_at | 10946574 | creatine kinase, brain [Mus musculus] | Ckb |
| 20 | 97983_s_at | 6678179 | syntaxin binding protein 1; unc18 homolog (C. elegans); UNC-18 homolog | Stxbp1 |
| | | | (C. | |
| 21 | 100510_at | 15809030 | synuclein, beta [Mus musculus] | Sncb |
| 22 | 93362_at | 6753074 | adaptor protein complex AP-2, mu1; adaptor-related protein complex AP-2, | Ap2m1 |
| | | | mu1; | |
| | | | tyrosine 3-monooxygenase/tryptophan 5-monooxygenase activation protein, | |
| 23 | 97544_at | 6756041 | zeta | Ywhaz |
| | AFFX-GapdhMur/- | | | |
| 24 | M32599_3_st | 6679937 | glyceraldehyde-3-phosphate dehydrogenase [Mus musculus] | |
| 25 | 100551_r_at | 16716343 | cytochrome c oxidase, subunit VIc [Mus musculus] | Cox6c |
| 26 | 99124_at | 9507187 | fractured callus expressed transcript 1; Fracture Callus 1; small zinc | Fxc1 |
| 27 | 92876_at | 6754814 | NADH dehydrogenase (ubiquinone) Fe—S protein 4; NADH dehydrogenase | Ndufs4 |
| | | | (ubiquinone) | |
| 28 | 96760_at | 7305573 | translocase of inner mitochondrial membrane 10 homolog [Mus musculus] | Timm10 |
| 29 | 94421_r_at | 6681031 | cryptochrome 1 (photolyase-like) [Mus musculus] | Cry1 |
| 30 | 93359_at | 18859597 | RIKEN cDNA 1810004I06 [Mus musculus] | 1810004I06Rik |
| 31 | 98832_at | 6678417 | thyroid peroxidase [Mus musculus] | Tpo |
| 32 | 96857_at | 6680816 | complement component 1, q subcomponent binding protein [Mus musculus] | C1qbp |
| 33 | 98117_at | 9506911 | NADH dehydrogenase (ubiquinone) 1 alpha subcomplex, 1 (7.5 kD, MWFE); | Ndufa1 |
| | | | NADH | |
| 34 | 100046_at | 6678952 | methylenetetrahydrofolate dehydrogenase (NAD+ dependent), | Mthfd2 |
| 35 | 103806_at | 6678716 | low density lipoprotein receptor-related protein 5; low density | Lrp5 |
| 36 | 97372_at | 18875324 | DAZ associated protein 1 [Mus musculus] | Dazap1 |
| 37 | 102416_at | 6681097 | cytochrome P450, family 17, subfamily a, polypeptide 1; cytochrome P450, | Cyp17 |
| | | | 17; | |
| 38 | 94850_at | 12331400 | acyl-Coenzyme A thioesterase 3, mitochondrial; MT-ACT48, p48 [Mus | Acate3-pending |
| | | | musculus] | |
| 39 | 103471_at | 31981207 | RIKEN cDNA 4432405K22 [Mus musculus] | 4432405K22Rik |
| 40 | 92810_at | 21704122 | pyruvate dehydrogenase kinase, isoenzyme 3 [Mus musculus] | Pdk3 |
| 41 | 93062_at | 31560438 | mitochondrial ribosomal protein L39; ribosomal protein, mitochondrial, L5 | Mrpl39 |
| | | | [Mus | |
| 42 | 97884_at | 17157979 | mitochondrial ribosomal protein S11 [Mus musculus] | Mrps11 |
| 43 | 94420_f_at | 6681031 | cryptochrome 1 (photolyase-like) [Mus musculus] | Cry1 |
| 44 | 99027_at | 31981887 | Bcl2-like [Mus musculus] | Bcl2l |
| 45 | 100619_r_at | 22094075 | solute carrier family 25, member 5; adenine nucleotide translocator 2, | Slc25a5 |
| 46 | 102007_at | 31542950 | holocytochrome c synthetase [Mus musculus] | Hccs |
| 47 | 95354_at | 7657583 | solute carrier family 25 (mitochondrial carrier; adenine nucleotide | Slc25a13 |
| 48 | 99543_s_at | 7304999 | deoxyguanosine kinase [Mus musculus] | Dguok |
| 49 | 98903_at | 21312028 | RIKEN cDNA 1110006I11 [Mus musculus] | 1110006I11Rik |
| 50 | 96032_at | 31982497 | ATP synthase, H+ transporting, mitochondrial F0 complex, subunit c (subunit | Atp5g1 |
| | | | 9), | |
| 51 | 95734_at | 31981470 | mitochondrial ribosomal protein L3 [Mus musculus] | Mrpl3 |
| 52 | 102128_f_at | 31981257 | mitochondrial ribosomal protein S25 [Mus musculus] | Mrps25 |
| 53 | 94210_at | 7305581 | translocase of inner mitochondrial membrane 9 homolog [Mus musculus] | Timm9 |
| 54 | 103622_at | 9910434 | malonyl-CoA decarboxylase [Mus musculus] | Mlycd |
| 55 | 96289_at | 12963591 | stomatin-like protein 2 [Mus musculus] | Stoml2 |
| 56 | AFFX- | 6679237 | pyruvate carboxylase; pyruvate decarboxylase [Mus musculus] | |
| | PyruCarbMur/- | | | |
| | L09192_5_at | | | |
| 57 | 95645_at | 21313484 | nitrogen fixation cluster-like [Mus musculus] | 2310020H20Rik |
| 58 | 96916_at | 13385266 | mitochondrial ribosomal protein L33 [Mus musculus] | Mrpl33 |
| 59 | 94012_at | 7305575 | translocase of inner mitochondrial membrane 13 homolog a [Mus musculus] | Timm13a |
| 60 | 93859_at | 19526984 | mitochondrial translational initiation factor 2 [Mus musculus] | 2410112O06Rik |
| 61 | 96202_at | 7106409 | solute carrier family 1, member 2; glial high affinity glutamate transporter | Slc1a2 |
| 62 | 96650_at | 7709988 | AU RNA-binding enoyl-coenzyme A hydratase; AU RNA-binding | Auh |
| | | | protein/enoyl-coenzyme | |
| 63 | 98120_at | 16716447 | mitochondrial ribosomal protein L27 [Mus musculus] | Mrpl27 |
| 64 | 93048_at | 8393156 | caseinolytic protease, ATP-dependent, proteolytic subunit homolog; | Clpp |
| | | | caseinolytic | |
| 65 | 94852_at | 31982332 | glutamate-ammonia ligase (glutamine synthase); glutamine synthetase [Mus | Glul |
| 66 | 98909_at | 13277380 | lipoic acid synthetase [Mus musculus] | Lias |
| 67 | 103646_at | 6681009 | carnitine acetyltransferase [Mus musculus] | Crat |
| 68 | 98984_f_at | 31981769 | glycerol-3-phosphate dehydrogenase 2; glycerol phosphate dehydrogenase | Gpd2 |
| | | | 1, | |
| 69 | 98099_at | 27753998 | nudix (nucleoside diphosphate linked moiety X)-type motif 9 [Mus musculus] | Nudt9 |
| 70 | 94897_at | 13540480 | glutathione peroxidase 4; sperm nuclei glutathione peroxidase; phospholipid | Gpx4 |
| 71 | 97369_g_at | 6753030 | A-kinase anchor protein 1; A kinase anchor protein [Mus musculus] | Akap1 |
| 72 | 99636_at | 14780884 | polymerase delta interacting protein 38 [Mus musculus] | 1300003F06Rik |
| 73 | 95215_f_at | 21070950 | ubiquitin C; polyubiquitin C [Mus musculus] | Ubc |
| 74 | 96095_i_at | 13195670 | RIKEN cDNA 2610207I16 [Mus musculus] | 2610207I16Rik |
| 75 | 93114_at | 10181184 | ATP synthase, H+ transporting, mitochondrial F0 complex, subunit f, isoform | Atp5j2 |
| | | | 2; | |
| 76 | 100527_at | 21311867 | hypothetical protein D11Ertd99e [Mus musculus] | D11Ertd99e |
| 77 | 92625_at | 6679078 | expressed in non-metastatic cells 2, protein; expressed in non-metastatic | Nme2 |
| | | | cells | |
| 78 | 96653_at | 21311883 | RIKEN cDNA 0610007O07 [Mus musculus] | 0610007O07Rik |
| 79 | 96856_at | 6680816 | complement component 1, q subcomponent binding protein [Mus musculus] | C1qbp |
| 80 | 98545_at | 6671622 | B-cell receptor-associated protein 37; repressor of estrogen receptor activity | Bcap37 |
| 81 | 96858_at | 6755004 | programmed cell death 8; programmed cell death 8 (apoptosis inducing | Pdcd8 |
| | | | factor); | |
| 82 | 94855_at | 6679299 | prohibitin [Mus musculus] | Phb |
| 83 | 99148_at | 33859554 | fumarate hydratase 1 [Mus musculus] | Fh1 |
| 84 | 96898_at | 33859512 | ATP synthase, H+ transporting, mitochondrial F0 complex, subunit b, | Atp5f1 |
| | | | isoform 1 | |
| 85 | AFFX-GapdhMur/- | 6679937 | glyceraldehyde-3-phosphate dehydrogenase [Mus musculus] | |
| | M32599_5_st | | | |
| 86 | AFFX-GapdhMur/- | 6679937 | glyceraldehyde-3-phosphate dehydrogenase [Mus musculus] | |
| | M32599_M_st | | | |
| 87 | 93392_at | 6678495 | uncoupling protein 3, mitochondrial [Mus musculus] | Ucp3 |
| 88 | 94379_at | 25031694 | kinesin family member 1B [Mus musculus] | Kif1b |
| 89 | 102426_at | 6753290 | calsequestrin 1 [Mus musculus] | Casq1 |
| 90 | 96801_at | 10946936 | adenylate kinase 1; cytosolic adenylate kinase [Mus musculus] | Ak1 |
| 91 | 96066_s_at | 31981562 | pyruvate kinase 3 [Mus musculus] | Pkm2 |
| 92 | 101214_f_at | 6679937 | glyceraldehyde-3-phosphate dehydrogenase [Mus musculus] | Gapd |
| 93 | AFFX-GapdhMur/- | 6679937 | glyceraldehyde-3-phosphate dehydrogenase [Mus musculus] | |
| | M32599_3_at | | | |
| 94 | AFFX-GapdhMur/- | 6679937 | glyceraldehyde-3-phosphate dehydrogenase [Mus musculus] | |
| | M32599_5_at | | | |
| 95 | AFFX-GapdhMur/- | 6679937 | glyceraldehyde-3-phosphate dehydrogenase [Mus musculus] | |
| | M32599_M_at | | | |
| 96 | 94279_at | 21536220 | RIKEN cDNA 0610008F14 [Mus musculus] | 0610008F14Rik |
| 97 | 95498_at | 13384968 | mitochondrial ribosomal protein S15 [Mus musculus] | Mrps15 |
| 98 | 98130_at | 9903609 | thioredoxin 2; thioredoxin nuclear gene encoding mitochondrial protein; | Txn2 |
| 99 | 96626_at | 27370092 | RIKEN cDNA 2300002G02 [Mus musculus] | 2300002G02Rik |
| 100 | 99658_f_at | 12963697 | RIKEN cDNA 1110025H10 [Mus musculus] | 1110025H10Rik |
| 101 | 97342_at | 13384894 | mitochondrial ribosomal protein S14 [Mus musculus] | Mrps14 |
| 102 | 95472_f_at | 13385726 | ubiquinol-cytochrome c reductase binding protein [Mus musculus] | 2210415M14Rik |
| 103 | 94062_at | 20900762 | NADH dehydrogenase (ubiquinone) flavoprotein 2 [Mus musculus] | Ndufv2 |
| 104 | 99661_r_at | 6680991 | cytochrome c oxidase, subunit VIIc; cytochrome c oxidase subunit VIIc [Mus | Cox7c |
| 105 | 95718_f_at | 13128954 | upregulated during skeletal muscle growth 5 [Mus musculus] | Usmg5 |
| 106 | 101580_at | 13384754 | cytochrome c oxidase subunit VIIb [Mus musculus] | Cox7b |
| 107 | 96887_at | 9506933 | neuronal protein 15.6 [Mus musculus] | Np15 |
| 108 | 96280_at | 31981600 | NADH dehydrogenase (ubiquinone) 1 alpha subcomplex, 2; NADH | Ndufa2 |
| | | | dehydrogenase | |
| 109 | 95131_f_at | 13386096 | NADH dehydrogenase (ubiquinone) 1 beta subcomplex, 2 [Mus musculus] | 1810011O01Rik |
| 110 | 95132_r_at | 13386096 | NADH dehydrogenase (ubiquinone) 1 beta subcomplex, 2 [Mus musculus] | 1810011O01Rik |
| 111 | 99660_f_at | 6680991 | cytochrome c oxidase, subunit VIIc; cytochrome c oxidase subunit VIIc [Mus | Cox7c |
| 112 | 93014_at | 31980744 | ATP synthase, H+ transporting, mitochondrial F0 complex, subunit g; F1F0- | Atp5l |
| | | | ATP | |
| 113 | 99678_f_at | 31980744 | ATP synthase, H+ transporting, mitochondrial F0 complex, subunit g; F1F0- | Atp5l |
| | | | ATP | |
| 114 | 97512_at | 21312554 | RIKEN cDNA 2010107E04 [Mus musculus] | 2010107E04Rik |
| 115 | 100550_f_at | 16716343 | cytochrome c oxidase, subunit VIc [Mus musculus] | Cox6c |
| 116 | 93820_at | 31981830 | cytochrome c oxidase, subunit VIIa 2; cytochrome c oxidase subunit VIIa 3; | Cox7a2 |
| 117 | 99115_at | 21539599 | ubiquinol cytochrome c reductase hinge protein; mitochondrial hinge protein; | 2610041P16Rik |
| 118 | 94909_at | 13384854 | mitochondrial ribosomal protein S17 [Mus musculus] | Mrps17 |
| 119 | 96686_i_at | 13385436 | RIKEN cDNA 2010100O12 [Mus musculus] | 2010100O12Rik |
| 120 | 96687_f_at | 13385436 | RIKEN cDNA 2010100O12 [Mus musculus] | 2010100O12Rik |
| 121 | 94526_at | 19527228 | DNA segment, Chr 10, ERATO Doi 214, expressed [Mus musculus] | D10Ertd214e |
| 122 | 97880_at | 21313536 | dihydrolipoamide S-succinyltransferase (E2 component of 2-oxo-glutarate | 4930529O08Rik |
| | | | complex) | |
| 123 | 96096_f_at | 13195670 | RIKEN cDNA 2610207I16 [Mus musculus] | 2610207I16Rik |
| 124 | 94866_at | 13384844 | mitochondrial ribosomal protein S16 [Mus musculus] | Mrps16 |
| 125 | 93582_at | 20587962 | demethyl-Q 7 [Mus musculus] | Coq7 |
| 126 | 94860_at | 33468943 | translocator of inner mitochondrial membrane a; translocator of inner | Timm17a |
| 127 | 100892_at | 31980802 | NADH dehydrogenase (ubiquinone) 1 alpha subcomplex, assembly factor 1; | Ndufaf1 |
| | | | NADH | |
| 128 | 102097_f_at | 21539587 | NADH-ubiquinone oxidoreductase B9 subunit; Complex I-B9; CI-B9 [Mus | 1010001M12Rik |
| | | | musculus] | |
| 129 | 97874_at | 33859744 | RIKEN cDNA 1500032D16 [Mus musculus] | 1500032D16Rik |
| 130 | 93562_at | 13385054 | NADH dehydrogenase (ubiquinone) 1 beta subcomplex 3 [Mus musculus] | 2700033I16Rik |
| 131 | 94534_at | 18250284 | isocitrate dehydrogenase 3 (NAD+) alpha [Mus musculus] | Idh3a |
| 132 | 98929_at | 13384742 | RIKEN cDNA 1110018B13 [Mus musculus] | 1110018B13Rik |
| 133 | 95058_f_at | 21312594 | RIKEN cDNA 2610205H19; EST AA108335 [Mus musculus] | 2610205H19Rik |
| 134 | 99666_at | 13385942 | citrate synthase [Mus musculus] | Cs |
| 135 | 94080_at | 20908717 | succinate dehydrogenase Fp subunit [Mus musculus] | Sdha |
| 136 | 93029_at | 6680345 | isocitrate dehydrogenase 3 (NAD+), gamma [Mus musculus] | Idh3g |
| 137 | 94912_at | 17505220 | mitochondrial ribosomal protein S21 [Mus musculus] | Mrps21 |
| 138 | 93531_at | 21312012 | NADH dehydrogenase (ubiquinone) 1 alpha subcomplex, 8 [Mus musculus] | 0610033L03Rik |
| 139 | 93754_at | 7949037 | enoyl coenzyme A hydratase 1, peroxisomal; peroxisomal/mitochondrial | Ech1 |
| | | | dienoyl-CoA | |
| 140 | 92581_at | 6680618 | acetyl-Coenzyme A dehydrogenase, medium chain [Mus musculus] | Acadm |
| 141 | 96112_at | 31981826 | electron transferring flavoprotein, alpha polypeptide; Alpha-ETF [Mus | Etfa |
| | | | musculus] | |
| 142 | 97869_at | 21313290 | electron transferring flavoprotein, dehydrogenase [Mus musculus] | 0610010I20Rik |
| 143 | 95072_at | 13385006 | cytochrome c-1 [Mus musculus] | Cyc1 |
| 144 | 96267_at | 19526814 | NADH dehydrogenase (ubiquinone) flavoprotein 1; NADH dehydrogenase | Ndufv1 |
| | | | flavoprotein | |
| 145 | 101989_at | 13384794 | ubiquinol-cytochrome c reductase core protein 1 [Mus musculus] | Uqcrc1 |
| 146 | 94806_at | 18152793 | pyruvate dehydrogenase (lipoamide) beta [Mus musculus] | Pdhb |
| 147 | 93815_at | 21313618 | RIKEN cDNA 0610041L09 [Mus musculus] | 0610041L09Rik |
| 148 | 96268_at | 9845299 | succinate-CoA ligase, GDP-forming, alpha subunit; succinyl-CoA synthetase | Suclg1 |
| | | | [Mus | |
| 149 | 102749_at | 6753504 | cytochrome c oxidase, subunit VIIa 1; cytochrome c oxidase subunit VIIa 1 | Cox7a1 |
| | | | [Mus | |
| 150 | 95698_at | 13385322 | NADH dehydrogenase (ubiquinone) 1 beta subcomplex, 7 [Mus musculus] | 1110002H15Rik |
| 151 | 93119_at | 6753500 | cytochrome c oxidase, subunit Vb [Mus musculus] | Cox5b |
| 152 | 96909_at | 27754007 | NADH dehydrogenase (ubiquinone)⁻1, alpha/beta subcomplex, 1 [Mus | 2610003B19Rik |
| | | | musculus] | |
| 153 | 99128_at | 20070412 | ATP synthase, H+ transporting, mitochondrial F1 complex, O subunit [Mus | Atp5o |
| 154 | 100753_at | 6680748 | ATP synthase, H+ transporting, mitochondrial F1 complex, alpha subunit, | Atp5a1 |
| | | | isoform | |
| 155 | 93596_i_at | 13385484 | ATP synthase, H+ transporting, mitochondrial F1 complex, epsilon subunit; | 2410043G19Rik |
| | | | ATP | |
| 156 | 93844_at | 21539585 | low molecular mass ubiquinone-binding protein; ubiquinol-cytochrome c | Uqcrb |
| | | | reductase | |
| 157 | 96915_f_at | 21539587 | NADH-ubiquinone oxidoreductase B9 subunit; Complex I-B9; CI-B9 [Mus | 1010001M12Rik |
| | | | musculus] | |
| 158 | 99618_at | 13385112 | ubiquinol-cytochrome c reductase subunit [Mus musculus] | 0710008D09Rik |
| 159 | 100079_at | 29789148 | NADH dehydrogenase (ubiquinone) 1 beta subcomplex, 9 [Mus musculus] | Ndufb9 |
| 160 | 93581_at | 13385558 | NADH dehydrogenase (ubiquinone) 1 beta subcomplex 8 [Mus musculus] | 2900010I05Rik |
| 161 | 96870_at | 18079339 | aconitase 2, mitochondrial [Mus musculus] | Aco2 |
| 162 | 98102_at | 6679261 | pyruvate dehydrogenase E1 alpha 1; pyruvate dehydrogenase E1alpha | Pdha1 |
| | | | subunit [Mus | |
| 163 | 95425_at | 31982520 | acetyl-Coenzyme A dehydrogenase, long-chain [Mus musculus] | Acadl |
| 164 | 96913_at | 21704100 | hydroxyacyl-Coenzyme A dehydrogenase/3-ketoacyl-Coenzyme A | 4930479F15Rik |
| 165 | 93972_at | 23346461 | NADH dehydrogenase (ubiquinone) Fe—S protein 2; NADH-coenzyme Q | Ndufs2 |
| | | | reductase [Mus | |
| 166 | 94216_at | 13384690 | succinate dehydrogenase complex, subunit C, integral membrane protein | 0610010E03Rik |
| | | | [Mus | |
| 167 | 97502_at | 31982856 | dihydrolipoamide dehydrogenase [Mus musculus] | Dld |
| 168 | 92574_at | 27229021 | RIKEN cDNA 3110001M13 [Mus musculus] | 3110001M13Rik |
| 169 | 102000_f_at | 22267442 | RIKubiquinol cytochrome c reductase core protein 2 [Mus musculus] | 1500004O06Rik |
| 170 | 96321_at | 13384720 | NADH dehydrogenase (ubiquinone) 1 alpha subcomplex, 9 [Mus musculus] | 1010001N11Rik |
| 171 | 97201_s_at | 13386100 | NADH dehydrogenase (ubiquinone) 1 alpha subcomplex, 5 [Mus musculus] | 2900002J19Rik |
| 172 | 93764_at | 12963633 | genes associated with retinoid-IFN-induced mortality 19 [Mus musculus] | Grim19-pending |
| 173 | 97307_f_at | 27754144 | NADH dehydrogenase (ubiquinone) 1 beta subcomplex, 5; NADH | Ndufb5 |
| | | | dehydrogenase | |
| 174 | 92798_at | 11602916 | ATP synthase, H+ transporting, mitochondrial F1 complex, gamma | Atp5c1 |
| | | | polypeptide 1; F1 | |
| 175 | 92799_g_at | 11602916 | ATP synthase, H+ transporting, mitochondrial F1 complex, gamma | Atp5c1 |
| | | | polypeptide 1; F1 | |
| 176 | 93572_at | 21704020 | NADH dehydrogenase (ubiquinone) Fe—S protein 1 [Mus musculus] | — |
| 177 | 93780_at | 13385260 | thioesterase superfamily member 2 [Mus musculus] | 0610006O17Rik |
| 178 | 99593_at | 19527334 | NADH dehydrogenase (ubiquinone) Fe—S protein 5; NADH dehydrogenase | Ndufs5 |
| | | | Fe—S protein | |
| 179 | 96746_at | 31542559 | dihydrolipoamide S-acetyltransferase (E2 component of pyruvate | Dlat |
| | | | dehydrogenase | |
| 180 | 95441_at | 12025536 | translocase of inner mitochondrial membrane 23 homolog [Mus musculus] | Timm23 |
| 181 | 102049_at | 7305375 | pyruvate dehydrogenase kinase, isoenzyme 4; pyruvate dehydrogenase | Pdk4 |
| | | | kinase 4 [Mus | |
| 182 | 95485_at | 6680163 | L-3-hydroxyacyl-Coenzyme A dehydrogenase, short chain; hydroxylacyl- | Hadhsc |
| | | | Coenzyme A | |
| 183 | 95426_at | 29789289 | enoyl Coenzyme A hydratase, short chain, 1, mitochondrial [Mus musculus] | Echs1 |
| 184 | 95064_at | 29126205 | acetyl-Coenzyme A acyltransferase 2 (mitochondrial 3-oxoacyl-Coenzyme A | D18Ertd240e |
| 185 | 96947_at | 21312004 | RIKEN cDNA 0610009I16 [Mus musculus] | 0610009I16Rik |
| 186 | 96757_at | 20070420 | DNA segment, Chr 10, Johns Hopkins University 81 expressed [Mus | D10Jhu81e |
| | | | musculus] | |
| 187 | 98128_at | 7949005 | ATP synthase, H+ transporting, mitochondrial F0 complex, subunit F; | Atp5j |
| 188 | 94531_at | 33859690 | RIKEN cDNA 2310005O14 [Mus musculus] | 2310005O14Rik |
| 189 | 99667_at | 6753502 | cytochrome c oxidase, subunit VI a, polypeptide 2; subunit VIaH (heart-type) | Cox6a2 |
| 190 | 102402_at | 6679957 | glioblastoma amplified [Mus musculus] | Gbas |
| 191 | 99631_f_at | 6680988 | cytochrome c oxidase, subunit VI a, polypeptide 1; subunit VIaL (liver-type) | Cox6a1 |
| 192 | 96670_at | 21313138 | glutathione S-transferase class kappa [Mus musculus] | 0610025I19Rik |
| 193 | 94940_at | 31980706 | methylcrotonoyl-Coenzyme A carboxylase 1 (alpha) [Mus musculus] | Mccc1 |
| 194 | AFFX- | 6679237 | pyruvate carboxylase; pyruvate decarboxylase [Mus musculus] | |
| | PyruCarbMur/- | | | |
| | L09192_MB_at | | | |
| 195 | AFFX- | 6679237 | pyruvate carboxylase; pyruvate decarboxylase [Mus musculus] | |
| | PyruCarbMur/- | | | |
| | L09192_3_at | | | |
| 196 | 93308_s_at | 6679237 | pyruvate carboxylase; pyruvate decarboxylase [Mus musculus] | Pcx |
| 197 | 103401_at | 31982522 | acyl-Coenzyme A dehydrogenase, short chain; acetyl-Coenzyme A | Acads |
| | | | dehydrogenase, | |
| 198 | 94807_at | 23943838 | solute carrier family 25, member 1; DiGeorge syndrome gene j; solute carrier | Slc25a1 |
| 199 | 97248_at | 6681137 | diazepam binding inhibitor; acyl-CoA binding protein; diazepam-binding | Dbi |
| | | | inhibitor | |
| 200 | 94507_at | 31560705 | fatty acid Coenzyme A ligase, long chain 2; acetyl-Coenzyme A synthetase; | Facl2 |
| 201 | 104057_at | 13277394 | GrpE-like 1, mitochondrial [Mus musculus] | Grpel1 |
| 202 | 97279_at | 21704140 | 3-hydroxyisobutyrate dehydrogenase, mitochondrial precursor; EST | AI265272 |
| | | | AI265272; | |
| 203 | 99613_at | 6678970 | methylmalonyl-coenzyme A mutase [Mus musculus] | Mut |
| 204 | 96035_at | 31982494 | branched chain ketoacid dehydrogenase E1, alpha polypeptide; BCKAD | Bckdha |
| | | | E1[a] [Mus | |
| 205 | 101045_at | 7949047 | hydroxyacyl-Coenzyme A dehydrogenase type II; hydroxyacyl-Coenzyme A | Hadh2 |
| 206 | 98966_at | 6753610 | dihydrolipoamide branched chain transacylase E2; BCKAD E2 [Mus | Dbt |
| | | | musculus] | |
| 207 | 104212_at | 21389320 | leucine-rich PPR motif-containing protein; leucine rich protein LRP130 [Mus | 3110001K13Rik |
| 208 | 92845_at | 18266680 | 3-oxoacid CoA transferase [Mus musculus] | Oxct |
| 209 | 99009_at | 31543330 | nicotinamide nucleotide transhydrogenase [Mus musculus] | Nnt |
| 210 | 97367_at | 6753030 | A-kinase anchor protein 1; A kinase anchor protein [Mus musculus] | Akap1 |
| 211 | 93042_at | 31981875 | benzodiazepine receptor, peripheral [Mus musculus] | Bzrp |
| 212 | 92754_at | 6679767 | ferredoxin reductase [Mus musculus] | Fdxr |
| 213 | 92587_at | 6679765 | ferredoxin 1; ADRENODOXIN [Mus musculus] | Fdx1 |
| 214 | 92213_at | 31543776 | steroidogenic acute regulatory protein [Mus musculus] | Star |
| 215 | 96256_at | 6680690 | peroxiredoxin 3; anti-oxidant protein 1; mitochondrial Trx dependent peroxide | Prdx3 |
| 216 | 92829_at | 6680309 | heat shock protein 1 (chaperonin 10); heat shock 10 kDa protein 1 | Hspe1 |
| | | | (chaperonin | |
| 217 | 93277_at | 31981679 | heat shock protein 1 (chaperonin); heat shock protein, 60 kDa; heat shock | Hspd1 |
| | | | 60 kDa | |
| 218 | 100977_at | 27369966 | RIKEN cDNA D530020C15 [Mus musculus] | D530020C15Rik |
| 219 | 101096_s_at | 6754160 | HS1 binding protein [Mus musculus] | Hs1bp1 |
| 220 | 95065_at | 6754846 | nitrogen fixation gene, yeast homolog 1; nifS-like (sic) [Mus musculus] | Nfs1 |
| 221 | AFFX- | 6679237 | pyruvate carboxylase; pyruvate decarboxylase [Mus musculus] | |
| | PyruCarbMur/- | | | |
| | L09192_MA_at | | | |
| 222 | 98137_at | 6671680 | carbonic anhydrase 5a, mitochondrial; carbonic anhydrase 5, mitochondrial; | Car5a |
| 223 | 98459_at | 6677943 | serine hydroxymethyl transferase 1 (soluble) [Mus musculus] | Shmt1 |
| 224 | 92586_at | 6680027 | glutamate dehydrogenase [Mus musculus] | Glud |
| 225 | 97450_s_at | 20070418 | aldehyde dehydrogenase family 7, member A1; aldehyde dehydrogenase 7 | Aldh7a1 |
| | | | family, | |
| 226 | 97515_at | 31982273 | hydroxysteroid (17-beta) dehydrogenase 4; hydroxysteroid 17-beta | Hsd17b4 |
| | | | dehydrogenase | |
| 227 | 103085_at | 7305137 | heme binding protein 1; heme-binding protein; p22 HBP; heme-binding | Hebp1 |
| | | | protein 1 | |
| 228 | 98533_at | 13385268 | cytochrome b-5 [Mus musculus] | Cyb5 |
| 229 | 104086_at | 21311901 | dimethylglycine dehydrogenase precursor [Mus musculus] | 1200014D15Rik |
| 230 | 96890_at | 13385298 | RIKEN cDNA 1300002A08 [Mus musculus] | 1300002A08Rik |
| 231 | 93026_at | 31981068 | microsomal glutathione S-transferase 1 [Mus musculus] | Mgst1 |
| 232 | 96763_at | 20149748 | sarcosine dehydrogenase [Mus musculus] | Sardh |
| 233 | 93278_at | 28545662 | sterol carrier protein 2, liver [Mus musculus] | Scp2 |
| 234 | 101515_at | 7656855 | acyl-Coenzyme A oxidase 1, palmitoyl; acyl-Coenzyme A oxidase; Acyl-CoA | Acox1 |
| | | | oxidase | |
| 235 | 93625_at | 7709978 | alanine-glyoxylate aminotransferase; alanine-glyoxylate aminotransferase 1 | Agxt |
| | | | [Mus | |
| 236 | 96326_at | 22122769 | tyrosine aminotransferase [Mus musculus] | Tat |
| 237 | 101910_f_at | 13654245 | major urinary protein 1 [Mus musculus] | Mup1 |
| 238 | 92606_at | 6678509 | urate oxidase; uricase [Mus musculus] | Uox |
| 239 | 102096_f_at | 13654245 | major urinary protein 1 [Mus musculus] | Mup1 |
| 240 | 93320_at | 27804309 | carnitine palmitoyltransferase 1, liver; L-CPT I [Mus musculus] | Cpt1a |
| 241 | 96057_at | 6753036 | aldehyde dehydrogenase 2, mitochondrial [Mus musculus] | Aldh2 |
| 242 | 96058_s_at | 6753036 | aldehyde dehydrogenase 2, mitochondrial [Mus musculus] | Aldh2 |
| 243 | 92800_i_at | 11602916 | ATP synthase, H+ transporting, mitochondrial F1 complex, gamma | Atp5c1 |
| | | | polypeptide 1; F1 | |
| 244 | 100617_at | 22094075 | solute carrier family 25, member 5; adenine nucleotide translocator 2, | Slc25a5 |
| 245 | 100618_f_at | 22094075 | solute carrier family 25, member 5; adenine nucleotide translocator 2, | Slc25a5 |
| 246 | 97207_f_at | 6678760 | lysophospholipase 1; phospholipase 1a; lysophopholipase 1 [Mus musculus] | Lypla1 |
| 247 | 98473_at | 6753110 | arginase type II [Mus musculus] | Arg2 |
| 248 | 98112_r_at | 31981147 | leucine aminopeptidase 3; leucine aminopeptidase [Mus musculus] | Lap3 |
| 249 | 100633_at | 19526848 | RIKEN cDNA 2810484M10 [Mus musculus] | 2810484M10Rik |
| 250 | 92848_at | 8393866 | ornithine aminotransferase [Mus musculus] | Oat |
| 251 | 104007_at | 6754952 | solute carrier family 25 (mitochondrial carrier; ornithine transporter), member | Slc25a15 |
| 252 | 96336_at | 13385454 | glycine amidinotransferase (L-arginine:glycine amidinotransferase) [Mus | Gatm |
| 253 | 93595_at | 6753448 | ceroid-lipofuscinosis neuronal 2 [Mus musculus] | Cln2 |
| 254 | 104153_at | 9789985 | isovaleryl coenzyme A dehydrogenase; isovaleryl dehydrogenase precursor | Ivd |
| | | | [Mus | |
| 255 | 94005_at | 20822904 | RIKEN cDNA 3110004O18 [Mus musculus] | 3110004O18Rik |
| 256 | 103881_at | 22203753 | inorganic pyrophosphatase 2 [Mus musculus] | 1110013G13Rik |
| 257 | 101944_at | 6678760 | lysophospholipase 1; phospholipase 1a; lysophopholipase 1 [Mus musculus] | Lypla1 |
| 258 | 101945_g_at | 6678760 | lysophospholipase 1; phospholipase 1a; lysophopholipase 1 [Mus musculus] | Lypla1 |
| 259 | 101946_at | 6678760 | lysophospholipase 1; phospholipase 1a; lysophopholipase 1 [Mus musculus] | Lypla1 |
| 260 | 99112_at | 7305501 | solute carrier family 25 (mitochondrial carrier; dicarboxylate transporter), | Slc25a10 |
| 261 | 99521_at | 6753022 | adenylate kinase 4 [Mus musculus] | Ak4 |
| 262 | 96069_at | 27659728 | aldo-keto reductase family 7, member A5 (aflatoxin aldehyde reductase); | Afar |
| 263 | 96231_at | 21624609 | RIKEN cDNA 2010012D11 [Mus musculus] | 2010012D11Rik |
| 264 | 97525_at | 6680139 | glycerol kinase [Mus musculus] | Gyk |
| 265 | 102192_r_at | 31982720 | SA rat hypertension-associated homolog [Mus musculus] | Sah |
| 266 | 93435_at | 6753572 | cytochrome P450, family 24, subfamily a, polypeptide 1; cytochrome P450, | Cyp24 |
| | | | 24; | |
| 267 | 99959_at | 6753022 | adenylate kinase 4 [Mus musculus] | Ak4 |
| 268 | 98123_at | 6754408 | kynurenine aminotransferase II [Mus musculus] | Kat2 |
| 269 | 96629_at | 14861848 | DNA segment, Chr 7, Roswell Park 2 complex, expressed; androgen | D7Rp2e |
| | | | regulated gene | |
| 270 | 92869_at | 6680291 | hydroxysteroid dehydrogenase-4, delta-3-beta; 3-beta-hydroxysteroid | Hsd3b4 |
| 271 | 96910_at | 22122743 | hypothetical protein MGC37245 [Mus musculus] | MGC37245 |
| 272 | 96938_at | 19482166 | kidney expressed gene 1 [Mus musculus] | Keg1 |
| 273 | 95588_at | 6678766 | alpha-methylacyl-CoA racemase; alpha-methylacyl-Coenzyme A racemase; | Amacr |
| 274 | 97316_at | 31541815 | L-specific multifunctional beta-oxdiation protein [Mus musculus] | 1300002P22Rik |
| 275 | 97258_at | 21703764 | lactamase, beta 2 [Mus musculus] | Cgi-83-pending |
| 276 | 97257_at | 21703764 | lactamase, beta 2 [Mus musculus] | Cgi-83-pending |
| 277 | 96048_at | 6680277 | heat-responsive protein 12 [Mus musculus] | Hrsp12 |
| 278 | 103389_at | 31980703 | aminoadipate-semialdehyde synthase; lysine oxoglutarate reductase, | Aass |
| | | | saccharopine | |
| 279 | 100967_at | 6755548 | solute carrier family 27 (fatty acid transporter), member 2; very long-chain | Slc27a2 |
| 280 | 96678_at | 13507612 | NADPH-dependent retinol dehydrogenase/reductase [Mus musculus] | D14Ucla2 |
| 281 | 92492_at | 23956104 | adenylate kinase 3 alpha-like; adenylate kinase 3 alpha like [Mus musculus] | Ak3l |
| 282 | 99659_r_at | 12963697 | RIKEN cDNA 1110025H10 [Mus musculus] | 1110025H10Rik |
| 283 | 102761_at | 29789124 | GrpE-like 2, mitochondrial [Mus musculus] | Grpel2 |
| 284 | 94961_at | 6753454 | caseinolytic protease X [Mus musculus] | Clpx |
| 285 | 103354_at | 10181116 | mitochondrial ribosomal protein S31; islet mitochondrial antigen, 38 kD [Mus | Mrps31 |
| 286 | 93506_at | 19526818 | solute carrier family 25 (mitochondrial carrier; phosphate carrier), member 3; | Slc25a3 |
| 287 | 93495_at | 7948999 | peroxiredoxin 4; antioxidant enzyme AOE372; Prx IV [Mus musculus] | Prdx4 |
| 288 | 97477_at | 7305579 | translocase of inner mitochondrial membrane 8 homolog b [Mus musculus] | Timm8b |
| 289 | 94515_at | 31981549 | sulfide quinone reductase-like; flavo-binding protein; sulfide | Sqrdl |
| 290 | 95660_at | 12963539 | HSCO protein [Mus musculus] | 0610025L15Rik |
| 291 | 92807_at | 6755911 | thioredoxin 1; thioredoxin [Mus musculus] | Txn1 |
| 292 | 93749_at | 27804325 | monoamine oxidase A [Mus musculus] | Maoa |
| 293 | 93984_at | 31982864 | ATPase inhibitor [Mus musculus] | Atpi |
| 294 | 96849_at | 7305577 | translocase of inner mitochondrial membrane 8 homolog a [Mus musculus] | Timm8a |
| 295 | 104283_at | 31981207 | RIKEN cDNA 4432405K22 [Mus musculus] | 4432405K22Rik |
| 296 | 99513_at | 6679369 | phospholipase A2, group IVA (cytosolic, calcium-dependent); phospholipase | Pla2g4a |
| | | | A2, | |
| 297 | 100957_at | 20916351 | single-stranded DNA binding protein 1 [Mus musculus] | — |
| 298 | 99335_at | 6754206 | hexokinase 1; downeast anemia [Mus musculus] | Hk1 |
| 299 | 92735_at | 7242175 | phospholipase A2, group IIA (platelets, synovial fluid); modifier of Min1; | Pla2g2a |
| 300 | 98902_at | 21312028 | RIKEN cDNA 1110006I11 [Mus musculus] | 1110006I11Rik |
| 301 | 94284_at | 19745150 | diaphorase 1 (NADH) [Mus musculus] | Dia1 |
| 302 | 92792_at | 31543920 | uncoupling protein 2, mitochondrial [Mus musculus] | Ucp2 |
| 303 | 95676_at | 18700024 | isocitrate dehydrogenase 3, beta subunit; isocitrate dehydrogenase 3 beta; | Idh3b |
| | | | N14A | |
| 304 | 99176_at | 10946808 | fibroblast growth factor (acidic) intracellular binding protein; aFGF | Fibp |
| 305 | 98613_at | 21313080 | RIKEN cDNA 2700085E05 [Mus musculus] | 2700085E05Rik |
| 306 | 96641_at | 6754870 | neighbor of Cox4 [Mus musculus] | Noc4 |
| 307 | 97825_at | 11528520 | p53 apoptosis effector related to Pmp22; p53 apoptosis-associated target | Perp-pending |
| | | | [Mus | |
| 308 | 92860_at | 6680993 | cytochrome c oxidase, subunit VIIIa; COX VIII-L [Mus musculus] | Cox8a |
| 309 | 99172_at | 6678303 | transcription factor A, mitochondrial [Mus musculus] | Tfam |
| 310 | 99836_at | 20867579 | cytochrome P450, 40 (25-hydroxyvitamin D3 1 alpha-hydroxylase) [Mus | Cyp40 |
| | | | musculus] | |
| 311 | 103043_at | 6754760 | mature T-cell proliferation 1 [Mus musculus] | Mtcp1 |
| 312 | 104102_at | 31980991 | protease, serine, 25; serine protease OMI [Mus musculus] | Prss25 |
| 313 | 97398_at | 28077029 | RIKEN cDNA 9130022B02 [Mus musculus] | 9130022B02Rik |
| 314 | 96353_at | 13384766 | RIKEN cDNA 1110021D01 [Mus musculus] | 11100021D01Rik |
| 315 | 100300_at | 31542440 | cytochrome b-245, beta polypeptide [Mus musculus] | Cybb |
| 316 | 99114_r_at | 13385090 | cytochrome c oxidase, subunit VIb [Mus musculus] | 2010000G05Rik |
| 317 | 96255_at | 6753200 | BCL2/adenovirus E1B 19 kDa-interacting protein 3-like; BCL2/adenovirus | Bnip3l |
| | | | E1B 19 | |
| 318 | 92768_s_at | 33859502 | aminolevulinic acid synthase 2, erythroid; erythroid-specific ALAS; | Alas2 |
| 319 | 100414_s_at | 6754732 | myeloperoxidase [Mus musculus] | Mpo |
| 320 | 92595_r_at | 20452466 | ferrochelatase [Mus musculus] | Fech |
| 321 | 98505_i_at | 6681007 | coproporphyrinogen oxidase; clone 560 [Mus musculus] | Cpo |
| 322 | 98506_r_at | 6681007 | coproporphyrinogen oxidase; clone 560 [Mus musculus] | Cpo |
| 323 | 104234_at | 31981257 | mitochondrial ribosomal protein S25 [Mus musculus] | Mrps25 |
| 324 | 97373_at | 21313024 | solute carrier family 25 (mitochondrial deoxynucleotide carrier), member 19 | Slc25a19 |
| | | | [Mus | |
| 325 | 94501_at | 13507712 | sphingosine-1-phosphate phosphatase 1; sphingosine-1-phosphate | — |
| | | | phosphatase [Mus | |
| 326 | 101557_at | 6753164 | branched chain ketoacid dehydrogenase kinase; branched chain keto acid | Bckdk |
| 327 | 100443_at | 33859514 | branched chain aminotransferase 2, mitochondrial [Mus musculus] | Bcat2 |
| 328 | 94034_at | 27229283 | small fragment nuclease [Mus musculus] | Smfn |
| 329 | 102058_at | 29789253 | mitochondrial ribosomal protein L9 [Mus musculus] | Mrpl9 |
| 330 | 103045_at | 6754760 | mature T-cell proliferation 1 [Mus musculus] | Mtcp1 |
| 331 | 93836_at | 6753198 | BCL2/adenovirus E1B 19 kDa-interacting protein 1, NIP3; BCL2/adenovirus | Bnip3 |
| | | | E1B 19 | |
| 332 | 99544_at | 7304999 | deoxyguanosine kinase [Mus musculus] | Dguok |
| 333 | 96848_at | 14916467 | inositol polyphosphate-5-phosphatase E; inositol polyphosphate-5- | Inpp5e |
| | | | phosphatase, 72 | |
| 334 | 102659_at | 31560609 | ceroid lipofuscinosis, neuronal 3, juvenile (Batten, Spielmeyer-Vogt disease) | Cln3 |
| 335 | 94541_at | 21314826 | NADH: ubiquinone oxidoreductase B15 subunit [Mus musculus] | 0610006N12Rik |
| 336 | 97368_at | 6753030 | A-kinase anchor protein 1; A kinase anchor protein [Mus musculus] | Akap1 |
| 337 | 96745_at | 31542559 | dihydrolipoamide S-acetyltransferase (E2 component of pyruvate | Dlat |
| | | | dehydrogenase | |
| 338 | 95607_at | 10946984 | START domain containing 3; es64 protein; steroidogenic acute regulatory | Stard3 |
| | | | protein | |
| 339 | 101407_at | 6679863 | frataxin [Mus musculus] | Frda |
| 340 | 95896_at | 6680991 | cytochrome c oxidase, subunit VIIc; cytochrome c oxidase subunit VIIc [Mus | — |
| 341 | 101356_at | 10835111 | thymidine kinase 2, mitochondrial; thymidine kinase 2 [Mus musculus] | Tk2 |
| 342 | 100059_at | 22094077 | cytochrome b-245, alpha polypeptide; cytochrome beta-558; p22 phox [Mus | Cyba |
| 343 | 93536_at | 6680770 | Bcl2-associated X protein [Mus musculus] | Bax |
| 344 | 101036_at | 13324686 | translocase of outer mitochondrial membrane 20 homolog [Mus musculus] | 1810060K07Rik |
| 345 | 97472_at | 29789024 | solute carrier family 25 (mitochondrial carrier; peroxisomal membrane | Slc25a17 |
| | | | protein), | |
| 346 | 92494_at | 6753058 | annexin A10 [Mus musculus] | Anxa10 |
| 347 | 96028_at | 9055178 | brain protein 44-like; apoptosis-regulating basic protein [Mus musculus] | Brp44l |
| 348 | 94254_at | 7304963 | chloride intracellular channel 4 (mitochondrial) [Mus musculus] | Clic4 |
| 349 | 94255_g_at | 7304963 | chloride intracellular channel 4 (mitochondrial) [Mus musculus] | Clic4 |
| 350 | 94256_at | 7304963 | chloride intracellular channel 4 (mitochondrial) [Mus musculus] | Clic4 |
| 351 | 99141_at | 6806917 | GM2 ganglioside activator protein [Mus musculus] | Gm2a |
| 352 | 101055_at | 6679437 | protective protein for beta-galactosidase [Mus musculus] | Ppgb |
| 353 | 92633_at | 11968166 | cathepsin Z preproprotein; cathepsin Z precursor; cathepsin X [Mus | Ctsz |
| | | | musculus] | |
| 354 | 102328_at | 20847456 | caspase 8 [Mus musculus] | Casp8 |
| 355 | 103608_at | 22267456 | RIKEN cDNA 2810431B21 [Mus musculus] | 2810431B21Rik |
| 356 | 93699_at | 7657467 | polymerase (DNA directed), gamma 2, accessory subunit; mitochondrial | Polg2 |
| | | | polymerase | |
| 357 | 96287_at | 21281687 | deoxyuridine triphosphatase [Mus musculus] | Dutp |
| 358 | 94283_at | 13385752 | mitochondrial ribosomal protein L49; neighbor of fau 1 [Mus musculus] | Mrpl49 |
| 359 | 98547_at | 6755360 | mitochondrial ribosomal protein S12; ribosomal protein, mitochondrial, S12; | Mrps12 |
| 360 | 93251_at | 6679066 | 4-nitrophenylphosphatase domain and non-neuronal SNAP25-like protein | Nipsnap1 |
| | | | homolog 1 | |
| 361 | 100589_at | 21313262 | inner membrane protein, mitochondrial [Mus musculus] | Immt |
| 362 | 104132_at | 6754870 | neighbor of Cox4 [Mus musculus] | Noc4 |
| 363 | 94368_at | 31088872 | suppressor of var1, 3-like 1 [Mus musculus] | — |
| 364 | 96036_at | 13384998 | tetratricopeptide repeat domain 11 [Mus musculus] | 2010003O14Rik |
| 365 | 100335_at | 6680758 | ATPase, Cu++ transporting, beta polypeptide; Wilson protein; toxic milk [Mus | Atp7b |
| 366 | 103683_at | 9910194 | dihydroorotate dehydrogenase [Mus musculus] | Dhodh |
| 367 | 97256_at | 27228985 | RIKEN cDNA 2410011G03 [Mus musculus] | 2410011G03Rik |
| 368 | 102031_at | 6755334 | ribonuclease H1 [Mus musculus] | Rnaseh1 |
| 369 | 96906_at | 18079334 | ethanol induced 6 [Mus musculus] | Etohi6 |
| 370 | 93561_at | 27754146 | RIKEN cDNA 0710001P09 [Mus musculus] | 0710001P09Rik |
| 371 | 94962_g_at | 6753454 | caseinolytic protease X [Mus musculus] | Clpx |
| 372 | 98433_at | 31542228 | BH3 interacting domain death agonist [Mus musculus] | Bid |
| 373 | 96904_at | 30794474 | mitchondrial ribosomal protein S7; ribosomal protein, mitochondrial, S7 [Mus | Mrps7 |
| 374 | 103386_at | 18875408 | peroxisomal acyl-CoA thioesterase 1 [Mus musculus] | Pte1 |
| 375 | 93355_at | 6754036 | glutamate oxaloacetate transaminase 2, mitochondrial; mitochondrial | Got2 |
| | | | aspartate | |
| 376 | 98139_at | 6755963 | voltage-dependent anion channel 1 [Mus musculus] | Vdac1 |
| 377 | 95738_at | 24025659 | pyrroline-5-carboxylate synthetase; glutamate gamma-semialdehyde | Pycs |
| | | | synthetase [Mus | |
| 378 | 98298_at | 6753676 | dihydropyrimidinase-like 2; collapsin response mediator protein 2 [Mus | Dpysl2 |
| | | | musculus] | |
| 379 | 95603_at | 20070408 | glycine decarboxylase [Mus musculus] | D19Wsu57e |
| 380 | 97993_at | 6678519 | uroporphyrinogen III synthase; URO-synthase; uroporphyrinogen-III | Uros |
| | | | synthase; | |
| 381 | 99159_at | 19527310 | peptidylprolyl isomerase F (cyclophilin F); peptidyl-prolyl cis-trans isomerase; | AW457192 |
| 382 | 98118_at | 9506911 | NADH dehydrogenase (ubiquinone) 1 alpha subcomplex, 1 (7.5 kD, MWFE); | Ndufa1 |
| | | | NADH | |
| 383 | 98106_at | 19705563 | translocator of inner mitochondrial membrane 44 [Mus musculus] | Timm44 |
| 384 | 103625_at | 16905099 | AFG3(ATPase family gene 3)-like 1 [Mus musculus] | Afg3l1 |
| 385 | 92497_at | 9790129 | solute carrier family 22 member 4; solute carrier family (organic cation | Slc22a4 |
| 386 | 93385_at | 6679146 | nth (endonuclease III)-like 1; thymine glycol DNA glycosylase/AP lyase [Mus | Nthl1 |

TABLE 7
The 643 genes in the mitochondria expression neighborhood. For each gene, the Affymetrix probe-set ID, neighborhood index (N₁₀₀),
protein exemplar (if the gene was in mito-A), gene symbol, description, and electronic annotations are provided.

| | | | | | Electronic | |
|---|---|---|---|---|---|---|
| | | mito-A | Gene | | Annotations | |
| Probe Set | N₁₀₀ | Exemplar | Symbol | Title | INTERPRO | PFAM |
| 97201_s_at | 69 | 13386100 | 2900002J19Rik | RIKEN cDNA 2900002J19 gene | — | — |
| 102561_at | 68 | | — | — | — | — |
| 92574_at | 68 | 27229021 | 3110001M13Rik | RIKEN cDNA 3110001M13 gene | — | — |
| 96321_at | 68 | 13384720 | 1010001N11Rik | RIKEN cDNA 1010001N11 gene | — | — |
| 99128_at | 68 | 20070412 | Atp5o | ATP synthase, H+ transporting, | IPR000711 // H+- | OSCP // ATP |
| | | | | mitochondrial F1 complex, O | transporting two- | synthase delta |
| | | | | subunit | sector ATPase, delta | (OSCP) subunit; 5.6e−64 |
| | | | | | (OSCP) subunit | |
| 100892_at | 67 | 31980802 | Ndufaf1 | NADH dehydrogenase | — | — |
| | | | | (ubiquinone) 1 alpha subcomplex, | | |
| | | | | assembly factor 1 | | |
| 102000_f_at | 67 | 22267442 | 1500004O06Rik | RIKEN cDNA 1500004O06 gene | IPR001431 // | Peptidase_M16 // |
| | | | | | Insulinase-like | Insulinase (Peptidase |
| | | | | | peptidase, family | family M16); 1.5e−40 |
| | | | | | M16 /// IPR001478 // | |
| | | | | | PDZ/DHR/GLGF | |
| | | | | | domain | |
| 93764_at | 67 | 12963633 | Grim 19-pending | genes associated with retinoid- | — | — |
| | | | | IFN-induced mortality 19 | | |
| 96112_at | 67 | 31981826 | Etfa | electron transferring flavoprotein, | IPR001308 // | ETF_alpha // Electron |
| | | | | alpha polypeptide | Electron transfer | transfer flavoprotein |
| | | | | | flavoprotein, alpha | alpha subuni; 3.5e−149 |
| | | | | | subunit | |
| 96611_at | 67 | | 2010012C24Rik | RIKEN cDNA 2010012C24 gene | — | — |
| 97502_at | 67 | 31982856 | Dld | dihydrolipoamide dehydrogenase | IPR001327 // FAD- | pyr_redox_dim // |
| | | | | | dependent pyridine | Pyridine nucleotide- |
| | | | | | nucleotide-disulphide | disulphide |
| | | | | | oxidoreductase /// | oxidored; 2.5e−61 /// |
| | | | | | IPR004099 // | pyr_redox // Pyridine |
| | | | | | Pyridine nucleotide- | nucleotide-disulphide |
| | | | | | disulphide | oxidored; 1.2e−92 |
| | | | | | oxidoreductase | |
| | | | | | dimerisation domain | |
| | | | | | /// IPR000815 // | |
| | | | | | Mercuric reductase /// | |
| | | | | | IPR001100 // | |
| | | | | | Pyridine nucleotide- | |
| | | | | | disulphide | |
| | | | | | oxidoreductase, class I | |
| 99106_at | 67 | | Cops6 | COP9 (constitutive | IPR003640 // Mov34 | — |
| | | | | photomorphogenic) homolog, | family, subtype 2 /// | |
| | | | | subunit 6 (Arabidopsis thaliana) | IPR000555 // Mov34 | |
| | | | | | family | |
| 99618_at | 67 | 13385112 | 0710008D09Rik | RIKEN cDNA 0710008D09 gene | — | — |
| 100753_at | 66 | 6680748 | Atp5a1 | ATP synthase, H+ transporting, | IPR005294 // ATP | ATP-synt_ab_N // ATP |
| | | | | mitochondrial F1 complex, alpha | synthase F1, alpha | synthase alpha/beta |
| | | | | subunit, isoform 1 | subunit /// IPR000793 | family, beta-ba; 8.4e−19 |
| | | | | | // H+-transporting | /// ATP-synt_ab // |
| | | | | | two-sector ATPase, | ATP synthase |
| | | | | | alpha/beta subunit, | alpha/beta family, |
| | | | | | C-terminal /// | nucleot; 3e−162 /// |
| | | | | | IPR004100 // H+- | ATP-synt_ab_C // ATP |
| | | | | | transporting two- | synthase alpha/beta |
| | | | | | sector ATPase, | chain, C termin; 4e−37 |
| | | | | | alpha/beta subunit, N- | |
| | | | | | terminal /// | |
| | | | | | IPR000790 // H+- | |
| | | | | | transporting two- | |
| | | | | | sector ATPase, alpha | |
| | | | | | subunit, C-terminal /// | |
| | | | | | IPR000194 // H+- | |
| | | | | | transporting two- | |
| | | | | | sector ATPase, | |
| | | | | | alpha/beta subunit, | |
| | | | | | central region | |
| 102228_at | 66 | | Lat | linker for activation of T cells | — | — |
| 92581_at | 66 | 6680618 | Acadm | acetyl-Coenzyme A | IPR006089 // Acyl- | Acyl-CoA_dh_M // |
| | | | | dehydrogenase, medium chain | CoA dehydrogenase | Acyl-CoA |
| | | | | | /// IPR006092 // Acyl- | dehydrogenase, |
| | | | | | CoA dehydrogenase, | middle domain; 3.1e−66 |
| | | | | | N-terminal /// | /// Acyl-CoA_dh // |
| | | | | | IPR006091 // Acyl- | Acyl-CoA |
| | | | | | CoA dehydrogenase, | dehydrogenase, C- |
| | | | | | middle domain /// | terminal doma; 4.5e−68 |
| | | | | | IPR006090 // Acyl- | /// Acyl-CoA_dh_N // |
| | | | | | CoA dehydrogenase, | Acyl-CoA |
| | | | | | C-terminal | dehydrogenase, N- |
| | | | | | | terminal doma; 2.1e−53 |
| 94912_at | 66 | 17505220 | Mrps21 | mitochondrial ribosomal protein | IPR001911 // | — |
| | | | | S21 | Ribosomal protein | |
| | | | | | S21 | |
| 97307_f_at | 66 | 27754144 | Ndufb5 | NADH dehydrogenase | — | — |
| | | | | (ubiquinone) 1 beta subcomplex, 5 | | |
| 97914_at | 66 | | Hspa9a | heat shock protein, A | IPR002048 // | HSP70 // Hsp70 |
| | | | | | Calcium-binding EF- | protein; 0 |
| | | | | | hand /// IPR001023 // | |
| | | | | | Heat shock protein | |
| | | | | | Hsp70 | |
| 99666_at | 66 | 13385942 | Cs | citrate synthase | IPR002020 // Citrate | citrate_synt // Citrate |
| | | | | | synthase | synthase; 4.4e−233 |
| 100079_at | 65 | 29789148 | Ndufb9 | NADH dehydrogenase | — | — |
| | | | | (ubiquinone) 1 beta subcomplex, 9 | | |
| 93991_at | 65 | | Mor1 | malate dehydrogenase, | IPR001236 // | ldh_C // lactate/malate |
| | | | | mitochondrial | Lactate/malate | dehydrogenase, |
| | | | | | dehydrogenase /// | alpha/beta C-t; 2e−72 |
| | | | | | IPR001252 // Malate | /// ldh // lactate/malate |
| | | | | | dehydrogenase | dehydrogenase, NAD |
| | | | | | | binding do; 3.1e−73 |
| 94461_at | 65 | | Pbef-pending | pre-B-cell colony-enhancing factor | IPR002088 // Protein | — |
| | | | | | prenyltransferase, | |
| | | | | | alpha subunit | |
| 94907_f_at | 65 | | 1110001J03Rik | RIKEN cDNA 1110001J03 gene | — | — |
| 95053_s_at | 65 | | 0710008N11Rik | RIKEN cDNA 0710008N11 gene | IPR006058 // 2Fe—2S | fer2 // 2Fe—2S iron- |
| | | | | | Ferredoxin /// | sulfur cluster binding |
| | | | | | IPR001450 // 4Fe—4S | domain; 0.057 |
| | | | | | ferredoxin, iron-sulfur | |
| | | | | | binding domain /// | |
| | | | | | IPR001041 // | |
| | | | | | Ferredoxin /// | |
| | | | | | IPR004489 // | |
| | | | | | Succinate | |
| | | | | | dehydrogenase/fumarate | |
| | | | | | reductase iron- | |
| | | | | | sulfur protein | |
| 95072_at | 65 | 13385006 | Cyc1 | cytochrome c-1 | IPR002326 // | Cytochrome_C1 // |
| | | | | | Cytochrome c1 /// | Cytochrome C1 |
| | | | | | IPR000345 // | family; 6.4e−165 |
| | | | | | Cytochrome c heme- | |
| | | | | | binding site | |
| 98132_at | 65 | | Cycs | cytochrome c, somatic | — | cytochrome_c // |
| | | | | | | Cytochrome c; 3.9e−38 |
| 99140_at | 65 | | Mrpl16 | mitochondrial ribosomal protein | IPR000114 // | Ribosomal_L16 // |
| | | | | L16 | Ribosomal protein | Ribosomal protein |
| | | | | | L16 | L16; 1.9e−07 |
| 92799_g_at | 64 | 11602916 | Atp5c1 | ATP synthase, H+ transporting, | IPR000131 // H+- | ATP-synt // ATP |
| | | | | mitochondrial F1 complex, gamma | transporting two- | synthase; 6.9e−132 |
| | | | | polypeptide 1 | sector ATPase, | |
| | | | | | gamma subunit | |
| 93119_at | 64 | 6753500 | Cox5b | cytochrome c oxidase, subunit Vb | IPR002124 // | COX5B // Cytochrome |
| | | | | | Cytochrome c | c oxidase subunit |
| | | | | | oxidase, subunit Vb | Vb; 2.4e−58 |
| 93562_at | 64 | 13385054 | 2700033I16Rik | RIKEN cDNA 2700033I16 gene | — | — |
| 94080_at | 64 | 20908717 | Sdha | succinate dehydrogenase | IPR003952 // | — |
| | | | | complex, subunit A, flavoprotein | Fumarate | |
| | | | | (Fp) | reductase/succinate | |
| | | | | | dehydrogenase, | |
| | | | | | FAD-binding site /// | |
| | | | | | IPR001327 // FAD- | |
| | | | | | dependent pyridine | |
| | | | | | nucleotide-disulphide | |
| | | | | | oxidoreductase /// | |
| | | | | | IPR004112 // | |
| | | | | | Fumarate | |
| | | | | | reductase/succinate | |
| | | | | | dehydrogenase | |
| | | | | | flavoprotein, C- | |
| | | | | | terminal /// | |
| | | | | | IPR001100 // | |
| | | | | | Pyridine nucleotide- | |
| | | | | | disulphide | |
| | | | | | oxidoreductase, class | |
| | | | | | I /// IPR003953 // | |
| | | | | | Fumarate | |
| | | | | | reductase/succinate | |
| | | | | | dehydrogenase | |
| | | | | | flavoprotein, N- | |
| | | | | | terminal | |
| 95058_f_at | 64 | 21312594 | 2610205H19Rik | RIKEN cDNA 2610205H19 gene | IPR005336 // Protein | UPF0041 // |
| | | | | | of unknown function | Uncharacterised |
| | | | | | UPF0041 | protein family |
| | | | | | | (UPF0041); 1.5e−33 |
| 95132_r_at | 64 | 13386096 | 1810011O01Rik | RIKEN cDNA 1810011O01 gene | — | — |
| 96291_f_at | 64 | | — | ESTs, Highly similar to | — | — |
| | | | | NUMM_MOUSE NADH- | | |
| | | | | ubiquinone oxidoreductase 13 kDa- | | |
| | | | | A subunit (Complex I-13 KD- | | |
| | | | | A) (CI-13 KD-A) [M. musculus] | | |
| 96899_at | 64 | | Ndufs3 | NADH dehydrogenase | IPR001268 // NADH | — |
| | | | | (ubiquinone) Fe—S protein 3 | dehydrogenase | |
| | | | | | (ubiquinone), 30 kDa | |
| | | | | | subunit | |
| 96909_at | 64 | 27754007 | 2610003B19Rik | RIKEN cDNA 2610003B19 gene | IPR003231 // Acyl | pp-binding // |
| | | | | | carrier protein (ACP) | Phosphopantetheine |
| | | | | | /// IPR002048 // | attachment site; 1.6e−17 |
| | | | | | Calcium-binding EF- | |
| | | | | | hand /// IPR006162 // | |
| | | | | | Phosphopantetheine | |
| | | | | | attachment site /// | |
| | | | | | IPR006163 // | |
| | | | | | Phosphopantetheine- | |
| | | | | | binding domain | |
| 97869_at | 64 | 21313290 | 0610010I20Rik | RIKEN cDNA 0610010I20 gene | IPR000103 // | — |
| | | | | | Pyridine nucleotide- | |
| | | | | | disulphide | |
| | | | | | oxidoreductase, | |
| | | | | | class-II | |
| 100432_f_at | 63 | | Mdfi | MyoD family inhibitor | — | — |
| 100628_at | 63 | | Ndufc1 | NADH dehydrogenase | — | — |
| | | | | (ubiquinone) 1, subcomplex | | |
| | | | | unknown, 1 | | |
| 101525_at | 63 | | 0610011B04Rik | RIKEN cDNA 0610011B04 gene | — | — |
| 101989_at | 63 | 13384794 | Uqcrc1 | ubiquinol-cytochrome c reductase | IPR001431 // | Peptidase_M16 // |
| | | | | core protein 1 | Insulinase-like | Insulinase (Peptidase |
| | | | | | peptidase, family | family M16); 2e−71 |
| | | | | | M16 | |
| 93581_at | 63 | 13385558 | 2900010I05Rik | RIKEN cDNA 2900010I05 gene | — | — |
| 93582_at | 63 | 20587962 | Coq7 | demethyl-Q 7 | IPR004916 // | COQ7 // Ubiquinone |
| | | | | | Ubiquinone | biosynthesis protein |
| | | | | | biosyntheis protein | COQ7; 2.9e−107 |
| | | | | | COQ7 | |
| 93815_at | 63 | 21313618 | 0610041L09Rik | RIKEN cDNA 0610041L09 gene | — | — |
| 93972_at | 63 | 23346461 | Ndufs2 | NADH dehydrogenase | IPR001135 // NADH- | complex1_49 Kd // |
| | | | | (ubiquinone) Fe—S protein 2 | ubiquinone | Respiratory-chain |
| | | | | | oxidoreductase, | NADH |
| | | | | | chain 49 kDa | dehydrogenase, |
| | | | | | | 4; 3.2e−205 |
| 94078_at | 63 | | 1110020P15Rik | RIKEN cDNA 1110020P15 gene | — | — |
| 94216_at | 63 | 13384690 | 0610010E03Rik | RIKEN cDNA 0610010E03 gene | IPR000701 // | Sdh_cyt // Succinate |
| | | | | | Succinate | dehydrogenase |
| | | | | | dehydrogenase, | cytochrome b |
| | | | | | cytochrome b subunit | subunit; 1.6e−44 |
| 94526_at | 63 | 19527228 | D10Ertd214e | DNA segment, Chr 10, ERATO | — | — |
| | | | | Doi 214, expressed | | |
| 94566_at | 63 | | G2a-pending | G protein-coupled receptor G2A | IPR005388 // G2A | 7tm_1 // 7 |
| | | | | | lysophosphatidylcholine | transmembrane |
| | | | | | receptor /// | receptor (rhodopsin |
| | | | | | IPR000276 // | family); 6.7e−38 |
| | | | | | Rhodopsin-like | |
| | | | | | GPCR superfamily | |
| 95517_i_at | 63 | | BC004004 | cDNA sequence BC004004 | — | — |
| 95652_at | 63 | | Ndufa7 | NADH dehydrogenase | — | — |
| | | | | (ubiquinone) 1 alpha subcomplex, | | |
| | | | | 7 (B14.5a) | | |
| 96042_at | 63 | | Sod2 | superoxide dismutase 2, | IPR001189 // | sodfe_C // |
| | | | | mitochondrial | Manganese and iron | Iron/manganese |
| | | | | | superoxide | superoxide |
| | | | | | dismutase | dismutases, C- |
| | | | | | | term; 1.8e−77 /// sodfe |
| | | | | | | // Iron/manganese |
| | | | | | | superoxide |
| | | | | | | dismutases, alpha-; |
| | | | | | | 1.5e−47 |
| 96082_at | 63 | | Mrpl30 | mitochondrial ribosomal protein | IPR000517 // | — |
| | | | | L30 | Ribosomal protein | |
| | | | | | L30 | |
| 96267_at | 63 | 19526814 | Ndufv1 | NADH dehydrogenase | IPR001949 // | Complex1_51K // |
| | | | | (ubiquinone) flavoprotein 1 | Respiratory-chain | Respiratory-chain |
| | | | | | NADH | NADH dehydrogenase |
| | | | | | dehydrogenase, 51 kDa | 51; 5.4e−183 |
| | | | | | subunit | |
| 96292_r_at | 63 | | — | ESTs, Highly similar to | — | — |
| | | | | NUMM_MOUSE NADH- | | |
| | | | | ubiquinone oxidoreductase 13 kDa- | | |
| | | | | A subunit (Complex I-13 KD- | | |
| | | | | A) (CI-13 KD-A) [M. musculus] | | |
| 96900_at | 63 | | AI267078 | expressed sequence AI267078 | — | — |
| 96913_at | 63 | 21704100 | 4930479F15Rik | RIKEN cDNA 4930479F15 gene | IPR002155 // | thiolase_C // Thiolase, |
| | | | | | Thiolase /// | C-terminal |
| | | | | | IPR000408 // | domain; 1.1e−78 /// |
| | | | | | Regulator of | thiolase // Thiolase, N- |
| | | | | | chromosome | terminal domain; 1.4e−131 |
| | | | | | condensation, RCC1 | |
| 96915_f_at | 63 | 21539587 | 1010001M12Rik | RIKEN cDNA 1010001M12 gene | — | — |
| 97874_at | 63 | 33859744 | 1500032D16Rik | RIKEN cDNA 1500032D16 gene | — | — |
| 99150_at | 63 | | Ict1 | immature colon carcinoma | IPR000352 // Class I | RF-1 // Peptidyl-tRNA |
| | | | | transcript 1 | peptide chain release | hydrolase domain; 7e−30 |
| | | | | | factor domain | |
| 93029_at | 62 | 6680345 | Idh3g | isocitrate dehydrogenase 3 | IPR001804 // | isodh // |
| | | | | (NAD+), gamma | Isocitrate/isopropylmalate | Isocitrate/isopropylmalate |
| | | | | | dehydrogenase | dehydrogenase; 4.7e−85 |
| | | | | | /// IPR004434 // | |
| | | | | | Isocitrate | |
| | | | | | dehydrogenase NAD- | |
| | | | | | dependent, | |
| | | | | | mitochondrial | |
| 93844_at | 62 | 21539585 | Uqcrb | ubiquinol-cytochrome c reductase | IPR004205 // UcrQ | UcrQ // UcrQ |
| | | | | binding protein | family | family; 1.9e−45 |
| 94005_at | 62 | 20822904 | 3110004O18Rik | RIKEN cDNA 3110004O18 gene | IPR001431 // | — |
| | | | | | Insulinase-like | |
| | | | | | peptidase, family | |
| | | | | | M16 | |
| 95472_f_at | 62 | 13385726 | 2210415M14Rik | RIKEN cDNA 2210415M14 gene | IPR003197 // | UCR_14 kD // |
| | | | | | Cytochrome bd | Ubiquinol-cytochrome |
| | | | | | ubiquinol oxidase, 14 kDa | C reductase complex |
| | | | | | subunit | 14k; 4.3e−58 |
| 96268_at | 62 | 9845299 | Suclg1 | succinate-CoA ligase, GDP- | IPR005811 // ATP- | ligase-CoA // CoA- |
| | | | | forming, alpha subunit | citrate lyase/succinyl- | ligase; 3.9e−65 /// |
| | | | | | CoA ligase /// | CoA_binding // CoA |
| | | | | | IPR005810 // | binding domain; 5e−72 |
| | | | | | Succinyl-CoA ligase, | |
| | | | | | alpha subunit /// | |
| | | | | | IPR003781 // CoA | |
| | | | | | Binding Domain | |
| 96626_at | 62 | 27370092 | 2300002G02Rik | RIKEN cDNA 2300002G02 gene | — | GTP_EFTU_D2 // |
| | | | | | | Elongation factor Tu |
| | | | | | | domain 2; 3.2e−24 /// |
| | | | | | | GTP_EFTU_D3 // |
| | | | | | | Elongation factor Tu |
| | | | | | | C-terminal |
| | | | | | | domain; 6.1e−41 /// |
| | | | | | | GTP_EFTU // |
| | | | | | | Elongation factor Tu |
| | | | | | | GTP binding |
| | | | | | | domain; 1.4e−89 |
| 96652_at | 62 | | Mrpl28 | mitochondrial ribosomal protein | — | — |
| | | | | L28 | | |
| 98102_at | 62 | 6679261 | Pdha1 | pyruvate dehydrogenase E1 alpha 1 | IPR001017 // | E1_dehydrog // |
| | | | | | Dehydrogenase, E1 | Dehydrogenase E1 |
| | | | | | component | component; 3.6e−183 |
| 102749_at | 61 | 6753504 | Cox7a1 | cytochrome c oxidase, | IPR003177 // | COX7a // Cytochrome |
| | | | | subunit VIIa 1 | Cytochrome c | c oxidase subunit |
| | | | | | oxidase, subunit VIIa | VIIa; 7.4e−56 |
| 103001_at | 61 | | Vegfb | vascular endothelial growth | IPR002400 // Growth | PDGF // Platelet- |
| | | | | factor B | factor, cystine knot /// | derived growth factor |
| | | | | | IPR000072 // | (PDGF); 4.3e−20 |
| | | | | | Platelet-derived | |
| | | | | | growth factor (PDGF) | |
| 93455_s_at | 61 | | Bmp4 | bone morphogenetic protein 4 | IPR001111 // | TGF-beta // |
| | | | | | Transforming growth | Transforming growth |
| | | | | | factor beta (TGFb), | factor beta like; 1.8e−62 |
| | | | | | N-terminal /// | /// |
| | | | | | IPR001839 // | TGFb_propeptide // |
| | | | | | Transforming growth | TGF-beta |
| | | | | | factor beta (TGFb) | propeptide; 2.4e−95 |
| 93501_f_at | 61 | | Sucla2 | succinate-Coenzyme A ligase, | IPR005811 // ATP- | — |
| | | | | ADP-forming, beta subunit | citrate lyase/succinyl- | |
| | | | | | CoA ligase /// | |
| | | | | | IPR005809 // | |
| | | | | | Succinyl-CoA | |
| | | | | | synthetase, beta | |
| | | | | | subunit /// IPR003135 | |
| | | | | | // ATP-dependent | |
| | | | | | carboxylate-amine | |
| | | | | | ligase-like, ATP- | |
| | | | | | grasp | |
| 94062_at | 61 | 20900762 | Ndufv2 | NADH dehydrogenase | IPR002023 // NADH | — |
| | | | | (ubiquinone) flavoprotein 2 | dehydrogenase | |
| | | | | | (ubiquinone), 24 kDa | |
| | | | | | subunit | |
| 94806_at | 61 | 18152793 | Pdhb | pyruvate dehydrogenase | IPR005476 // | transketolase_C // |
| | | | | (lipoamide) beta | Transketolase, C | Transketolase, C- |
| | | | | | terminal /// | terminal domain; 4.1e−55 |
| | | | | | IPR005475 // | /// transket_pyr // |
| | | | | | Transketolase, | Transketolase, |
| | | | | | central region | pyridine binding |
| | | | | | | domai; 1.5e−73 |
| 95698_at | 61 | 13385322 | 1110002H15Rik | RIKEN cDNA 1110002H15 gene | — | — |
| 99593_at | 61 | 19527334 | Ndufs5 | NADH dehydrogenase | — | — |
| | | | | (ubiquinone) Fe—S protein 5 | | |
| 100307_at | 60 | | — | Mus musculus 4 days neonate | — | — |
| | | | | male adipose cDNA, RIKEN full- | | |
| | | | | length enriched library, | | |
| | | | | clone: B430214H24 | | |
| | | | | product: nuclear factor I/X, full | | |
| | | | | insert sequence. | | |
| 102097_f_at | 60 | 21539587 | 1010001M12Rik | RIKEN cDNA 1010001M12 gene | — | — |
| 103406_at | 60 | | 2410004J02Rik | RIKEN cDNA 2410004J02 gene | IPR003593 // AAA | ATP-bind // Conserved |
| | | | | | ATPase /// | hypothetical ATP |
| | | | | | IPR004130 // | binding protein; 6.4e−114 |
| | | | | | Conserved | |
| | | | | | hypothetical ATP | |
| | | | | | binding protein | |
| 92798_at | 60 | 11602916 | Atp5c1 | ATP synthase, H+ transporting, | IPR000131 // H+- | ATP-synt // ATP |
| | | | | mitochondrial F1 complex, gamma | transporting two- | synthase; 6.9e−132 |
| | | | | polypeptide 1 | sector ATPase, | |
| | | | | | gamma subunit | |
| 93502_r_at | 60 | | Sucla2 | succinate-Coenzyme A ligase, | IPR005811 // ATP- | — |
| | | | | ADP-forming, beta subunit | citrate lyase/succinyl- | |
| | | | | | CoA ligase /// | |
| | | | | | IPR005809 // | |
| | | | | | Succinyl-CoA | |
| | | | | | synthetase, beta | |
| | | | | | subunit /// IPR003135 | |
| | | | | | // ATP-dependent | |
| | | | | | carboxylate-amine | |
| | | | | | ligase-like, ATP- | |
| | | | | | grasp | |
| 93572_at | 60 | 21704020 | — | Mus musculus, clone | IPR001467 // | fer2 // 2Fe—2S iron- |
| | | | | IMAGE: 1380460, mRNA | Prokaryotic | sulfur cluster binding |
| | | | | | molybdopterin | domain; 1.7e−11 |
| | | | | | oxidoreductase /// | |
| | | | | | IPR001041 // | |
| | | | | | Ferredoxin /// | |
| | | | | | IPR000283 // | |
| | | | | | Respiratory-chain | |
| | | | | | NADH | |
| | | | | | dehydrogenase 75 Kd | |
| | | | | | subunit | |
| 94537_at | 60 | | 1500001M02Rik | RIKEN cDNA 1500001M02 gene | IPR002048 // | efhand // EF |
| | | | | | Calcium-binding EF- | hand; 1.3e−13 |
| | | | | | hand | |
| 94860_at | 60 | 33468943 | Timm17a | translocator of inner mitochondrial | IPR005678 // | — |
| | | | | membrane 17 kDa, a | Mitochondrial import | |
| | | | | | inner membrane | |
| | | | | | translocase, subunit | |
| | | | | | Tim17 /// IPR003397 | |
| | | | | | // Mitochondrial | |
| | | | | | import inner | |
| | | | | | membrane | |
| | | | | | translocase, subunit | |
| | | | | | Tim17/22 | |
| 95483_at | 60 | | Psmd1 | proteasome (prosome, macropain) | — | — |
| | | | | 26S subunit, non-ATPase, 1 | | |
| 96686_i_at | 60 | 13385436 | 2010100O12Rik | RIKEN cDNA 2010100O12 gene | — | — |
| 99658_f_at | 60 | 12963697 | 1110025H10Rik | RIKEN cDNA 1110025H10 gene | IPR002529 // | FAA_hydrolase // |
| | | | | | Fumarylacetoacetate | Fumarylacetoacetate |
| | | | | | (FAA) hydrolase | (FAA) hydrolase |
| | | | | | | fam; 5.8e−79 |
| 99660_f_at | 60 | 6680991 | Cox7c | cytochrome c oxidase, subunit VIIc | IPR004202 // | COX7C // Cytochrome |
| | | | | | Cytochrome c | c oxidase subunit |
| | | | | | oxidase subunit VIIc | VIIc; 4e−33 |
| 101023_f_at | 59 | | 0610010E21Rik | RIKEN cDNA 0610010E21 gene | — | — |
| 101094_at | 59 | | Hig1-pending | hypoxia induced gene 1 | — | — |
| 102022_at | 59 | | 1110007A04Rik | RIKEN cDNA 1110007A04 gene | IPR004360 // | — |
| | | | | | Glyoxalase/Bleomycin | |
| | | | | | resistance | |
| | | | | | protein/dioxygenase | |
| | | | | | domain | |
| 92615_at | 59 | | 2010003O02Rik | RIKEN cDNA 2010003O02 gene | — | — |
| 93596_i_at | 59 | 13385484 | 2410043G19Rik | RIKEN cDNA 2410043G19 gene | — | — |
| 95485_at | 59 | 6680163 | Hadhsc | L-3-hydroxyacyl-Coenzyme A | IPR006180 // 3- | 3HCDH_N // 3- |
| | | | | dehydrogenase, short chain | hydroxyacyl-CoA | hydroxyacyl-CoA |
| | | | | | dehydrogenase /// | dehydrogenase, NAD |
| | | | | | IPR000205 // NAD | binding; 8.9e−105 /// |
| | | | | | binding site /// | 3HCDH // 3- |
| | | | | | IPR006108 // 3- | hydroxyacyl-CoA |
| | | | | | hydroxyacyl-CoA | dehydrogenase, C- |
| | | | | | dehydrogenase, C- | terminal; 2e−45 |
| | | | | | terminal domain /// | |
| | | | | | IPR006176 // 3- | |
| | | | | | hydroxyacyl-CoA | |
| | | | | | dehydrogenase, NAD | |
| | | | | | binding domain | |
| 96879_at | 59 | | Ogdh | oxoglutarate dehydrogenase | IPR001017 // | — |
| | | | | (lipoamide) | Dehydrogenase, E1 | |
| | | | | | component /// | |
| | | | | | IPR005475 // | |
| | | | | | Transketolase, | |
| | | | | | central region | |
| 103331_at | 58 | | C030006K11Rik | RIKEN cDNA C030006K11 gene | IPR000834 // Zinc | — |
| | | | | | carboxypeptidase A | |
| | | | | | metalloprotease | |
| | | | | | (M14) /// IPR002086 | |
| | | | | | // Aldehyde | |
| | | | | | dehydrogenase | |
| 92568_at | 58 | | Hkp1 | house-keeping protein 1 | IPR001737 // | RrnaAD // Ribosomal |
| | | | | | Ribosomal RNA | RNA adenine |
| | | | | | adenine dimethylase | dimethylase; 8.2e−06 |
| 93531_at | 58 | 21312012 | 0610033L03Rik | RIKEN cDNA 0610033L03 gene | — | — |
| 93787_f_at | 58 | | 1010001C05Rik | RIKEN cDNA 1010001C05 gene | — | — |
| 95736_at | 58 | | Mrpl4 | mitochondrial ribosomal protein L4 | IPR002136 // | Ribosomal_L4 // |
| | | | | | Ribosomal protein | Ribosomal protein |
| | | | | | L4/L1e | L4/L1 family; 5.1e−07 |
| 96687_f_at | 58 | 13385436 | 2010100O12Rik | RIKEN cDNA 2010100O12 gene | — | — |
| 96757_at | 58 | 20070420 | D10Jhu81e | DNA segment, Chr 10, Johns | IPR002818 // Family | DJ-1_PfpI // DJ-1/PfpI |
| | | | | Hopkins University 81 expressed | of unknown function | family; 2.3e−28 |
| | | | | | ThiJ/PfpI | |
| 99166_at | 58 | | 0610012G03Rik | RIKEN cDNA 0610012G03 gene | — | — |
| 102124_f_at | 57 | | Cox4a | cytochrome c oxidase, subunit IVa | IPR004203 // | COX4 // Cytochrome c |
| | | | | | Cytochrome c | oxidase subunit |
| | | | | | oxidase subunit IV | IV; 1.2e−68 |
| 95105_at | 57 | | 2010110M21Rik | RIKEN cDNA 2010110M21 gene | — | — |
| 95131_f_at | 57 | 13386096 | 1810011O01Rik | RIKEN cDNA 1810011O01 gene | — | — |
| 95425_at | 57 | 31982520 | Acadl | acetyl-Coenzyme A | IPR006089 // Acyl- | Acyl-CoA_dh_N // |
| | | | | dehydrogenase, long-chain | CoA dehydrogenase | Acyl-CoA |
| | | | | | /// IPR006092 // Acyl- | dehydrogenase, N- |
| | | | | | CoA dehydrogenase, | terminal doma; 9.6e−47 |
| | | | | | N-terminal /// | /// Acyl-CoA_dh // |
| | | | | | IPR006091 // Acyl- | Acyl-CoA |
| | | | | | CoA dehydrogenase, | dehydrogenase, C- |
| | | | | | middle domain /// | terminal doma; 1.2e−62 |
| | | | | | IPR006090 // Acyl- | /// Acyl-CoA_dh_M // |
| | | | | | CoA dehydrogenase, | Acyl-CoA |
| | | | | | C-terminal | dehydrogenase, |
| | | | | | | middle domain; 5.4e−61 |
| 96870_at | 57 | 18079339 | Aco2 | aconitase 2, mitochondrial | IPR000573 // | aconitase // Aconitase |
| | | | | | Aconitate hydratase, | family (aconitate |
| | | | | | C-terminal /// | hydratase); 2.1e−272 |
| | | | | | IPR002155 // | /// Aconitase_C // |
| | | | | | Thiolase /// | Aconitase C-terminal |
| | | | | | IPR001030 // | domain; 1.8e−86 |
| | | | | | Aconitate hydratase, | |
| | | | | | N-terminal | |
| 97880_at | 57 | 21313536 | 4930529O08Rik | RIKEN cDNA 4930529O08 gene | IPR001078 // | biotin_lipoyl // Biotin- |
| | | | | | Catalytic domain of | requiring |
| | | | | | components of | enzyme; 1.7e−27 /// 2- |
| | | | | | various | oxoacid_dh // 2-oxo |
| | | | | | dehydrogenase | acid dehydrogenases |
| | | | | | complexes /// | acyltransfera; 1.8e−132 |
| | | | | | IPR003016 // 2-oxo | |
| | | | | | acid dehydrogenase, | |
| | | | | | acyltransferase | |
| | | | | | component, lipoyl- | |
| | | | | | binding /// IPR000089 | |
| | | | | | // Biotin/lipoyl | |
| | | | | | attachment | |
| 99471_at | 57 | | AL022671 | expressed sequence AL022671 | IPR002913 // Lipid- | START // START |
| | | | | | binding START | domain; 1.5e−07 |
| 104212_at | 56 | 21389320 | 3110001K13Rik | RIKEN cDNA 3110001K13 gene | IPR002885 // PPR | PPR // PPR repeat; 3e−30 |
| | | | | | repeat | |
| 92763_at | 56 | | Abcb7 | ATP-binding cassette, sub-family | IPR003439 // ABC | — |
| | | | | B (MDR/TAP), member 7 | transporter /// | |
| | | | | | IPR003593 // AAA | |
| | | | | | ATPase /// | |
| | | | | | IPR001140 // ABC | |
| | | | | | transporter, | |
| | | | | | transmembrane | |
| | | | | | region | |
| 94534_at | 56 | 18250284 | Idh3a | isocitrate dehydrogenase 3 | IPR001804 // | isodh // |
| | | | | (NAD+) alpha | Isocitrate/isopropylmalate | Isocitrate/isopropylmalate |
| | | | | | dehydrogenase | dehydrogenase; 2.5e−173 |
| | | | | | /// IPR004434 // | |
| | | | | | Isocitrate | |
| | | | | | dehydrogenase NAD- | |
| | | | | | dependent, | |
| | | | | | mitochondrial | |
| 94780_at | 56 | | Zfp288 | zinc finger protein 288 | IPR000210 // | zf-C2H2 // Zinc finger, |
| | | | | | BTB/POZ domain /// | C2H2 type; 9.7e−32 /// |
| | | | | | IPR000822 // Zn- | BTB // BTB/POZ |
| | | | | | finger, C2H2 type | domain; 3.9e−27 |
| 95441_at | 56 | 12025536 | Timm23 | translocase of inner mitochondrial | IPR005681 // | — |
| | | | | membrane 23 homolog (yeast) | Mitochondrial import | |
| | | | | | inner membrane | |
| | | | | | translocase, subunit | |
| | | | | | Tim23 | |
| 95690_at | 56 | | 1110030L07Rik | RIKEN cDNA 1110030L07 gene | — | — |
| 96280_at | 56 | 31981600 | Ndufa2 | NADH dehydrogenase | — | — |
| | | | | (ubiquinone) 1 alpha | | |
| | | | | subcomplex, 2 | | |
| 96746_at | 56 | 31542559 | Dlat | dihydrolipoamide S- | IPR004167 // E3 | 2-oxoacid_dh // 2-oxo |
| | | | | acetyltransferase (E2 component | binding domain /// | acid dehydrogenases |
| | | | | of pyruvate dehydrogenase | IPR001078 // | acyltransfera; 3.8e−127 |
| | | | | complex) | Catalytic domain of | /// e3_binding // e3 |
| | | | | | components of | binding domain; 2.9e−19 |
| | | | | | various | /// biotin_lipoyl // |
| | | | | | dehydrogenase | Biotin-requiring |
| | | | | | complexes /// | enzyme; 3.8e−29 |
| | | | | | IPR001412 // | |
| | | | | | Aminoacyl-tRNA | |
| | | | | | synthetase, class I /// | |
| | | | | | IPR003016 // 2-oxo | |
| | | | | | acid dehydrogenase, | |
| | | | | | acyltransferase | |
| | | | | | component, lipoyl- | |
| | | | | | binding /// IPR000089 | |
| | | | | | // Biotin/lipoyl | |
| | | | | | attachment | |
| 96945_at | 56 | | Snap23 | synaptosomal-associated protein, | IPR000928 // SNAP- | SNAP-25 // SNAP-25 |
| | | | | 23 kD | 25 family /// | family; 1.3e−24 |
| | | | | | IPR000727 // Target | |
| | | | | | SNARE coiled-coil | |
| | | | | | domain | |
| 101472_s_at | 55 | | Pklr | pyruvate kinase liver and red blood | IPR001697 // | PK_C // Pyruvate |
| | | | | cell | Pyruvate kinase | kinase, alpha/beta |
| | | | | | | domain; 5.9e−71 /// PK |
| | | | | | | // Pyruvate kinase, |
| | | | | | | barrel domain; 1e−252 |
| 103261_at | 55 | | Gspt2 | G1 to phase transition 2 | IPR004160 // | GTP_EFTU // |
| | | | | | Elongation factor Tu, | Elongation factor Tu |
| | | | | | C-terminal /// | GTP binding |
| | | | | | IPR004161 // | domain; 8.1e−93 /// |
| | | | | | Elongation factor Tu, | GTP_EFTU_D3 // |
| | | | | | domain 2 /// | Elongation factor Tu |
| | | | | | IPR000795 // | C-terminal |
| | | | | | Elongation factor, | domain; 1.4e−30 /// |
| | | | | | GTP-binding | GTP_EFTU_D2 // |
| | | | | | | Elongation factor Tu |
| | | | | | | domain 2; 7.5e−11 |
| 103849_at | 55 | | Crkl | v-crk sarcoma virus CT10 | IPR001452 // SH3 | SH2 // SH2 |
| | | | | oncogene homolog (avian)-like | domain /// IPR000980 | domain; 5.7e−31 /// |
| | | | | | // SH2 motif | SH3 // SH3 |
| | | | | | | domain; 1.2e−20 |
| 93014_at | 55 | 31980744 | Atp5l | ATP synthase, H+ transporting, | — | — |
| | | | | mitochondrial F0 complex, | | |
| | | | | subunit g | | |
| 93780_at | 55 | 13385260 | 0610006O17Rik | RIKEN cDNA 0610006O17 gene | IPR003736 // | DUF157 // |
| | | | | | Phenylacetic acid | Uncharacterized |
| | | | | | degradation-related | protein Paal, |
| | | | | | protein | COG2050; 2.9e−10 |
| 94562_at | 55 | | Gnpat | glyceronephosphate O- | IPR002123 // | Acyltransferase // |
| | | | | acyltransferase | Phospholipid/glycerol | Acyltransferase; 6.2e−33 |
| | | | | | acyltransferase | |
| 95611_at | 55 | | Lpl | lipoprotein lipase | IPR002330 // | lipase // Lipase; 1.1e−173 |
| | | | | | Lipoprotein lipase /// | /// PLAT // |
| | | | | | IPR001024 // | PLAT/LH2 |
| | | | | | Lipoxygenase, LH2 | domain; 5.8e−37 |
| | | | | | domain /// IPR000734 | |
| | | | | | // Lipase /// | |
| | | | | | IPR000379 // | |
| | | | | | Esterase/lipase/thioesterase, | |
| | | | | | active site | |
| 95658_at | 55 | | Murr1 | U2af1-rs1 region 1 | — | — |
| 97422_at | 55 | | 2010002H18Rik | RIKEN cDNA 2010002H18 gene | IPR002300 // | — |
| | | | | | Aminoacyl-tRNA | |
| | | | | | synthetase, class Ia | |
| 94279_at | 54 | 21536220 | 0610008F14Rik | RIKEN cDNA 0610008F14 gene | IPR001469 // H+- | ATP-synt_DE // ATP |
| | | | | | transporting two- | synthase, |
| | | | | | sector ATPase, | Delta/Epsilon chain, |
| | | | | | delta/epsilon subunit | long; 0.011 /// ATP- |
| | | | | | | synt_DE_N // ATP |
| | | | | | | synthase, |
| | | | | | | Delta/Epsilon chain, |
| | | | | | | beta; 4.5e−31 |
| 94908_r_at | 54 | | 1110001J03Rik | RIKEN cDNA 1110001J03 gene | — | — |
| 98130_at | 54 | 9903609 | Txn2 | thioredoxin 2 | IPR000063 // | thiored // |
| | | | | | Thioredoxin type | Thioredoxin; 3.4e−28 |
| | | | | | domain /// IPR005746 | |
| | | | | | // Thioredoxin | |
| 98539_at | 54 | | Cops2 | COP9 (constitutive | IPR000717 // Domain | PCI // PCI |
| | | | | photomorphogenic) homolog, | in components of the | domain; 3.4e−25 |
| | | | | subunit 2 (Arabidopsis thaliana) | proteasome, COP9- | |
| | | | | | complex and eIF3 | |
| | | | | | (PCI) | |
| 98929_at | 54 | 13384742 | 1110018B13Rik | RIKEN cDNA 111018B13 gene | — | — |
| 99237_at | 54 | | U55872 | cDNA sequence U55872 | IPR001288 // | IF3 // Translation |
| | | | | | Initiation factor 3 | initiation factor IF- |
| | | | | | | 3; 2.5e−34 |
| 101045_at | 53 | 7949047 | Hadh2 | hydroxyacyl-Coenzyme A | IPR002198 // Short- | adh_short // short |
| | | | | dehydrogenase type II | chain | chain |
| | | | | | dehydrogenase/reductase | dehydrogenase; 7.4e−49 |
| | | | | | SDR /// | |
| | | | | | IPR002347 // | |
| | | | | | Glucose/ribitol | |
| | | | | | dehydrogenase | |
| 92625_at | 53 | 6679078 | Nme2 | expressed in non-metastatic cells | IPR000834 // Zinc | NDK // Nucleoside |
| | | | | 2, protein (NM23B) (nucleoside | carboxypeptidase A | diphosphate |
| | | | | diphosphate kinase) | metalloprotease | kinase; 1.9e−116 |
| | | | | | (M14) /// IPR001564 | |
| | | | | | // Nucleoside | |
| | | | | | diphosphate kinase | |
| | | | | | /// IPR003599 // | |
| | | | | | Immunoglobulin | |
| | | | | | subtype /// | |
| | | | | | IPR003598 // | |
| | | | | | Immunoglobulin C-2 | |
| | | | | | type /// IPR003006 // | |
| | | | | | Immunoglobulin/major | |
| | | | | | histocompatibility | |
| | | | | | complex /// | |
| | | | | | IPR003596 // | |
| | | | | | Immunoglobulin V- | |
| | | | | | type | |
| 93754_at | 53 | 7949037 | Ech1 | enoyl coenzyme A hydratase 1, | IPR001753 // Enoyl- | ECH // Enoyl-CoA |
| | | | | peroxisomal | CoA | hydratase/isomerase |
| | | | | | hydratase/isomerase | family; 1.4e−43 |
| 94829_at | 53 | | 1110020A09Rik | RIKEN cDNA 1110020A09 gene | — | — |
| 95646_at | 53 | | Cpt2 | carnitine palmitoyltransferase 2 | IPR000542 // | Carn_acyltransf // |
| | | | | | Acyltransferase | Choline/Carnitine o- |
| | | | | | ChoActase/COT/CPT | acyltransferase; 4.4e−289 |
| 99594_at | 53 | | Mrpl51 | mitochondrial ribosomal protein | — | — |
| | | | | L51 | | |
| 100886_f_at | 52 | | Mrpl45 | mitochondrial ribosomal protein | — | — |
| | | | | L45 | | |
| 94866_at | 52 | 13384844 | Mrps16 | mitochondrial ribosomal protein | IPR000307 // | Ribosomal_S16 // |
| | | | | S16 | Ribosomal protein | Ribosomal protein |
| | | | | | S16 | S16; 5.4e−17 |
| 94909_at | 52 | 13384854 | Mrps17 | mitochondrial ribosomal protein | IPR000266 // | Ribosomal_S17 // |
| | | | | S17 | Ribosomal protein | Ribosomal protein |
| | | | | | S17 | S17; 0.0021 |
| 95941_at | 52 | | AI853514 | expressed sequence AI853514 | IPR000569 // HECT | — |
| | | | | | domain (Ubiquitin- | |
| | | | | | protein ligase) | |
| 99613_at | 52 | 6678970 | Mut | methylmalonyl-Coenzyme A | IPR006100 // | MM_CoA_mutase // |
| | | | | mutase | Methylmalonyl-CoA | Methylmalonyl-CoA |
| | | | | | mutase subfamily /// | mutase; 0 /// B12- |
| | | | | | IPR006159 // | binding // B12 binding |
| | | | | | Methylmalonyl-CoA | domain; 1.7e−20 |
| | | | | | mutase, C-terminal /// | |
| | | | | | IPR006158 // | |
| | | | | | Coenzyme B12- | |
| | | | | | binding /// IPR006099 | |
| | | | | | // Methylmalonyl-CoA | |
| | | | | | mutase /// IPR006098 | |
| | | | | | // Methylmalonyl-CoA | |
| | | | | | mutase, N-terminal | |
| | | | | | domain | |
| 102624_at | 51 | | Stc2 | stanniocalcin 2 | IPR004978 // | Stanniocalcin // |
| | | | | | Stanniocalcin | Stanniocalcin |
| | | | | | | family; 5.7e−193 |
| 94327_at | 51 | | Mrps18a | mitochondrial ribosomal protein | IPR001648 // | Ribosomal_S18 // |
| | | | | S18A | Ribosomal protein | Ribosomal protein |
| | | | | | S18 | S18; 0.0013 |
| 94667_at | 51 | | — | ESTs | — | — |
| 94940_at | 51 | 31980706 | Mccc1 | methylcrotonoyl-Coenzyme A | IPR005482 // Biotin | CPSase_L_chain // |
| | | | | carboxylase 1 (alpha) | carboxylase, C- | Carbamoyl-phosphate |
| | | | | | terminal /// | synthase L |
| | | | | | IPR005481 // | chain,; 2.9e−53 /// |
| | | | | | Carbamoyl- | biotin_lipoyl // Biotin- |
| | | | | | phosphate | requiring |
| | | | | | synthetase large | enzyme; 3.5e−14 /// |
| | | | | | chain, N-terminal /// | Biotin_carb_C // Biotin |
| | | | | | IPR001882 // Biotin- | carboxylase C- |
| | | | | | requiring enzyme, | terminal domain; 1e−43 |
| | | | | | attachment site /// | /// CPSase_L_D2 // |
| | | | | | IPR000089 // | Carbamoyl-phosphate |
| | | | | | Biotin/lipoyl | synthase L |
| | | | | | attachment /// | chain,; 2.2e−100 |
| | | | | | IPR005479 // | |
| | | | | | Carbamoyl- | |
| | | | | | phosphate synthase | |
| | | | | | L chain, ATP-binding | |
| 96756_at | 51 | | 1110007M04Rik | RIKEN cDNA 1110007M04 gene | — | — |
| 96871_at | 51 | | 2310042G06Rik | RIKEN cDNA 2310042G06 gene | — | — |
| 98892_at | 51 | | Lpin1 | lipin 1 | — | — |
| 101867_at | 50 | | Gpam | glycerol-3-phosphate | IPR002123 // | Acyltransferase // |
| | | | | acyltransferase, mitochondrial | Phospholipid/glycerol | Acyltransferase; 5.3e−36 |
| | | | | | acyltransferase | |
| 94855_at | 50 | 6679299 | Phb | prohibitin | IPR001107 // Band 7 | Band_7 // SPFH |
| | | | | | protein /// IPR000163 | domain/Band 7 |
| | | | | | // Prohibitin | family; 3.7e−61 |
| 96744_at | 50 | | Acp6 | acid phosphatase 6, | IPR000560 // | acid_phosphat // |
| | | | | lysophosphatidic | Histidine acid | Histidine acid |
| | | | | | phosphatase | phosphatase; 2.4e−07 |
| 96858_at | 50 | 6755004 | Pdcd8 | programmed cell death 8 | IPR001327 // FAD- | pyr_redox // Pyridine |
| | | | | | dependent pyridine | nucleotide-disulphide |
| | | | | | nucleotide-disulphide | oxidoreducta; 2.6e−52 |
| | | | | | oxidoreductase /// | |
| | | | | | IPR001100 // | |
| | | | | | Pyridine nucleotide- | |
| | | | | | disulphide | |
| | | | | | oxidoreductase, class I | |
| 96898_at | 50 | 33859512 | Atp5f1 | ATP synthase, H+ transporting, | — | — |
| | | | | mitochondrial F0 complex, subunit | | |
| | | | | b, isoform 1 | | |
| 100550_f_at | 49 | 16716343 | Cox6c | cytochrome c oxidase, subunit VIc | IPR004204 // | COX6C // Cytochrome |
| | | | | | Cytochrome c | c oxidase subunit |
| | | | | | oxidase subunit VIc | VIc; 2.5e−50 |
| 103780_at | 49 | | 1700021F05Rik | RIKEN cDNA 1700021F05 gene | — | — |
| 104153_at | 49 | 9789985 | Ivd | isovaleryl coenzyme A | IPR006089 // Acyl- | Acyl-CoA_dh_N // |
| | | | | dehydrogenase | CoA dehydrogenase | Acyl-CoA |
| | | | | | /// IPR006092 // Acyl- | dehydrogenase, N- |
| | | | | | CoA dehydrogenase, | terminal doma; 4.7e−58 |
| | | | | | N-terminal /// | /// Acyl-CoA_dh // |
| | | | | | IPR006091 // Acyl- | Acyl-CoA |
| | | | | | CoA dehydrogenase, | dehydrogenase, C- |
| | | | | | middle domain /// | terminal doma; 3e−55 |
| | | | | | IPR006090 // Acyl- | /// Acyl-CoA_dh_M // |
| | | | | | CoA dehydrogenase, | Acyl-CoA |
| | | | | | C-terminal | dehydrogenase, |
| | | | | | | middle domain; 9.3e−71 |
| 92364_at | 49 | | Celsr2 | cadherin EGF LAG seven-pass G- | IPR002126 // | laminin_G // Laminin |
| | | | | type receptor 2 | Cadherin /// | G domain; 1.2e−18 /// |
| | | | | | IPR001881 // EGF- | EGF // EGF-like |
| | | | | | like calcium-binding | domain; 1.6e−21 /// |
| | | | | | /// IPR001368 // | GPS // Latrophilin/CL- |
| | | | | | TNFR/CD27/30/40/95 | 1-like GPS |
| | | | | | cysteine-rich region | domain; 1.3e−26 /// |
| | | | | | /// IPR000561 // EGF- | cadherin // Cadherin |
| | | | | | like domain /// | domain; 2.9e−209 /// |
| | | | | | IPR000742 // EGF- | 7tm_2 // 7 |
| | | | | | like domain, subtype | transmembrane |
| | | | | | 2 /// IPR000203 // | receptor (Secretin |
| | | | | | GPS domain /// | family); 1.8e−58 /// |
| | | | | | IPR000152 // | HRM // Hormone |
| | | | | | Aspartic acid and | receptor domain; 6.2e−17 |
| | | | | | asparagine | |
| | | | | | hydroxylation site /// | |
| | | | | | IPR002049 // | |
| | | | | | Laminin-type EGF- | |
| | | | | | like domain /// | |
| | | | | | IPR000832 // G- | |
| | | | | | protein coupled | |
| | | | | | receptors family 2 | |
| | | | | | (secretin-like) /// | |
| | | | | | IPR001791 // Laminin | |
| | | | | | G /// IPR001879 // | |
| | | | | | Hormone receptor, | |
| | | | | | extracellular | |
| 93399_at | 49 | | Rai2 | retinoic acid induced 2 | — | — |
| 93611_at | 49 | | Tbx6 | T-box 6 | IPR001699 // | T-box // T-box; 1.1e−125 |
| | | | | | Transcription factor, | |
| | | | | | T-box /// IPR002070 | |
| | | | | | // Transcription factor, | |
| | | | | | Brachyury | |
| 94531_at | 49 | 33859690 | 2310005O14Rik | RIKEN cDNA 2310005O14 gene | — | — |
| 96096_f_at | 49 | 13195670 | 2610207I16Rik | RIKEN cDNA 2610207I16 gene | IPR002198 // Short- | adh_short // short |
| | | | | | chain | chain |
| | | | | | dehydrogenase/reductase | dehydrogenase; 1.2e−29 |
| | | | | | SDR /// | /// SCP2 // SCP-2 |
| | | | | | IPR003033 // Sterol- | sterol transfer |
| | | | | | binding /// IPR002347 | family; 1.5e−27 |
| | | | | | // Glucose/ribitol | |
| | | | | | dehydrogenase | |
| 96261_at | 49 | | 2310028O11Rik | RIKEN cDNA 2310028O11 gene | — | — |
| 99148_at | 49 | 33859554 | Fh1 | fumarate hydratase 1 | IPR000362 // | — |
| | | | | | Fumarate lyase | |
| 104710_at | 48 | | Bak1 | BCL2-antagonist/killer 1 | IPR000712 // | Bcl-2 // Apoptosis |
| | | | | | Apoptosis regulator | regulator proteins, Bcl- |
| | | | | | Bcl-2 protein, BH /// | 2 family; 2.3e−39 |
| | | | | | IPR002475 // BCL2- | |
| | | | | | like apoptosis | |
| | | | | | inhibitor | |
| 96095_i_at | 48 | 13195670 | 2610207I16Rik | RIKEN cDNA 2610207I16 gene | IPR002198 // Short- | adh_short // short |
| | | | | | chain | chain |
| | | | | | dehydrogenase/reductase | dehydrogenase; 1.2e−29 |
| | | | | | SDR /// | /// SCP2 // SCP-2 |
| | | | | | IPR003033 // Sterol- | sterol transfer |
| | | | | | binding /// IPR002347 | family; 1.5e−27 |
| | | | | | // Glucose/ribitol | |
| | | | | | dehydrogenase | |
| 97397_at | 48 | | D5Ertd33e | DNA segment, Chr 5, ERATO Doi | IPR004033 // | Ubie_methyltran // |
| | | | | 33, expressed | UbiE/COQ5 | ubiE/COQ5 |
| | | | | | methyltransferase /// | methyltransferase |
| | | | | | IPR000051 // SAM | family; 1.4e−116 |
| | | | | | (and some other | |
| | | | | | nucleotide) binding | |
| | | | | | motif /// IPR004034 // | |
| | | | | | Ubiquinone/menaquinone | |
| | | | | | biosynthesis | |
| | | | | | methyltransferase /// | |
| | | | | | IPR001601 // Generic | |
| | | | | | methyltransferase | |
| 103294_at | 47 | | Rgs5 | regulator of G-protein signaling 5 | IPR000342 // | — |
| | | | | | Regulator of G | |
| | | | | | protein | |
| 103646_at | 47 | 6681009 | Crat | carnitine acetyltransferase | IPR000542 // | Carn_acyltransf // |
| | | | | | Acyltransferase | Choline/Carnitine o- |
| | | | | | ChoActase/COT/CPT | acyltransferase; 0 |
| 94508_at | 47 | | 1810020E01Rik | RIKEN cDNA 1810020E01 gene | — | — |
| 95939_i_at | 47 | | 9830126M18 | hypothetical protein 9830126M18 | — | — |
| 96035_at | 47 | 31982494 | Bckdha | branched chain ketoacid | IPR001017 // | E1_dehydrog // |
| | | | | dehydrogenase E1, alpha | Dehydrogenase, E1 | Dehydrogenase E1 |
| | | | | polypeptide | component | component; 1.8e−162 |
| 96296_at | 47 | | Mrpl15 | mitochondrial ribosomal protein | IPR001196 // | — |
| | | | | L15 | Ribosomal protein | |
| | | | | | L15 | |
| 96670_at | 47 | 21313138 | 0610025I19Rik | RIKEN cDNA 0610025I19 gene | IPR004287 // 2- | HCCA_isomerase // 2- |
| | | | | | hydroxychromene-2- | hydroxychromene-2- |
| | | | | | carboxylate | carboxylate |
| | | | | | isomerase | isomer; 1.8e−110 |
| 97796_at | 47 | | Crsp2 | cofactor required for Sp1 | — | — |
| | | | | transcriptional activation subunit 2 | | |
| 98128_at | 47 | 7949005 | Atp5j | ATP synthase, H+ transporting, | — | — |
| | | | | mitochondrial F0 complex, | | |
| | | | | subunit F | | |
| 100527_at | 46 | 21311867 | D11Ertd99e | DNA segment, Chr 11, ERATO | — | — |
| | | | | Doi 99, expressed | | |
| 101027_s_at | 46 | | Pttg1 | pituitary tumor-transforming 1 | — | — |
| 104215_at | 46 | | 9130025P16Rik | RIKEN cDNA 9130025P16 gene | — | — |
| 104767_f_at | 46 | | Mrps18a | mitochondrial ribosomal protein | IPR001648 // | Ribosomal_S18 // |
| | | | | S18A | Ribosomal protein | Ribosomal protein |
| | | | | | S18 | S18; 0.0013 |
| 93346_at | 46 | | Pgk1 | phosphoglycerate kinase 1 | IPR001576 // | PGK // |
| | | | | | Phosphoglycerate | Phosphoglycerate |
| | | | | | kinase | kinase; 8.4e−296 |
| 93539_at | 46 | | 1810004D07Rik | RIKEN cDNA 1810004D07 gene | — | — |
| 95498_at | 46 | 13384968 | Mrps15 | mitochondrial ribosomal protein | IPR005290 // | Ribosomal_S15 // |
| | | | | S15 | Ribosomal protein | Ribosomal protein |
| | | | | | S15, bacterial | S15; 1.1e−08 |
| | | | | | chloroplast and | |
| | | | | | mitochondrial type /// | |
| | | | | | IPR000589 // | |
| | | | | | Ribosomal protein | |
| | | | | | S15 | |
| 96947_at | 46 | 21312004 | 0610009I16Rik | RIKEN cDNA 0610009I16 gene | IPR000049 // | ETF_beta // Electron |
| | | | | | Electron transfer | transfer flavoprotein |
| | | | | | flavoprotein beta- | beta subunit; 3.3e−124 |
| | | | | | subunit /// IPR006162 | |
| | | | | | // | |
| | | | | | Phosphopantetheine | |
| | | | | | attachment site | |
| 103401_at | 45 | 31982522 | Acads | acetyl-Coenzyme A | IPR006089 // Acyl- | Acyl-CoA_dh_M // |
| | | | | dehydrogenase, short chain | CoA dehydrogenase | Acyl-CoA |
| | | | | | /// IPR006092 // Acyl- | dehydrogenase, |
| | | | | | CoA dehydrogenase, | middle domain; 9e−64 |
| | | | | | N-terminal /// | /// Acyl-CoA_dh_N // |
| | | | | | IPR006091 // Acyl- | Acyl-CoA |
| | | | | | CoA dehydrogenase, | dehydrogenase, N- |
| | | | | | middle domain /// | terminal doma; 1.9e−60 |
| | | | | | IPR006090 // Acyl- | /// Acyl-CoA_dh // |
| | | | | | CoA dehydrogenase, | Acyl-CoA |
| | | | | | C-terminal | dehydrogenase, C- |
| | | | | | | terminal doma; 3.9e−77 |
| 104057_at | 45 | 13277394 | Grpel1 | GrpE-like 1, mitochondrial | IPR000740 // GrpE | GrpE // GrpE; 3.8e−76 |
| | | | | | protein | |
| 95064_at | 45 | 29126205 | D18Ertd240e | DNA segment, Chr 18, ERATO | — | — |
| | | | | Doi 240, expressed | | |
| 96180_at | 45 | | Rgs5 | regulator of G-protein signaling 5 | IPR000342 // | — |
| | | | | | Regulator of G | |
| | | | | | protein | |
| 96887_at | 45 | 9506933 | Np15 | nuclear protein 15.6 | — | — |
| 97706_at | 45 | | — | ESTs | — | — |
| 96322_at | 44 | | Edf1 | endothelial differentiation-related | IPR001387 // Helix- | HTH_3 // Helix-turn- |
| | | | | factor 1 | turn-helix motif | helix; 1.2e−10 |
| 98527_at | 44 | | — | — | — | — |
| 102193_at | 43 | | Sah | SA rat hypertension-associated | IPR000873 // AMP- | AMP-binding // AMP- |
| | | | | homolog | dependent | binding enzyme; 1.2e−102 |
| | | | | | synthetase and ligase | |
| 93332_at | 43 | | Cd36 | CD36 antigen | IPR002159 // CD36 | CD36 // CD36 |
| | | | | | antigen /// IPR005428 | family; 1e−208 |
| | | | | | // Adhesion molecule | |
| | | | | | CD36 | |
| 93528_s_at | 43 | | Klf9 | Kruppel-like factor 9 | IPR000822 // Zn- | zf-C2H2 // Zinc finger, |
| | | | | | finger, C2H2 type | C2H2 type; 2.4e−21 |
| 93994_at | 43 | | Sycp3 | synaptonemal complex protein 3 | — | — |
| 95730_at | 43 | | Tce2 | T-complex expressed gene 2 | — | — |
| 96676_at | 43 | | 1810049H20Rik | RIKEN cDNA 1810049H20 gene | — | — |
| 97512_at | 43 | 21312554 | 2010107E04Rik | RIKEN cDNA 2010107E04 gene | — | — |
| 101078_at | 42 | | Bsg | basigin | IPR003599 // | — |
| | | | | | Immunoglobulin | |
| | | | | | subtype /// | |
| | | | | | IPR003006 // | |
| | | | | | Immunoglobulin/major | |
| | | | | | histocompatibility | |
| | | | | | complex | |
| 94365_at | 42 | | 1190005L05Rik | RIKEN cDNA 1190005L05 gene | IPR001310 // | — |
| | | | | | Histidine triad (HIT) | |
| | | | | | protein | |
| 94485_at | 42 | | Peci | peroxisomal delta3, delta2-enoyl- | IPR001753 // Enoyl- | ECH // Enoyl-CoA |
| | | | | Coenzyme A isomerase | CoA | hydratase/isomerase |
| | | | | | hydratase/isomerase | family; 3.2e−22 /// |
| | | | | | /// IPR000582 // Acyl- | ACBP // Acyl CoA |
| | | | | | coA-binding protein, | binding protein; 9.2e−41 |
| | | | | | ACBP | |
| 95056_r_at | 42 | | Tcte1l | t-complex-associated-testis- | IPR005334 // Tctex-1 | Tctex-1 // Tctex-1 |
| | | | | expressed 1-like | family | family; 5.5e−55 |
| 98966_at | 42 | 6753610 | Dbt | dihydrolipoamide branched chain | IPR004167 // E3 | e3_binding // e3 |
| | | | | transacylase E2 | binding domain /// | binding domain; 6.3e−18 |
| | | | | | IPR001078 // | /// 2-oxoacid_dh // |
| | | | | | Catalytic domain of | 2-oxo acid |
| | | | | | components of | dehydrogenases |
| | | | | | various | acyltransfera; 5.4e−108 |
| | | | | | dehydrogenase | /// biotin_lipoyl // |
| | | | | | complexes /// | Biotin-requiring |
| | | | | | IPR003016 // 2-oxo | enzyme; 2e−25 |
| | | | | | acid dehydrogenase, | |
| | | | | | acyltransferase | |
| | | | | | component, lipoyl- | |
| | | | | | binding /// IPR000089 | |
| | | | | | // Biotin/lipoyl | |
| | | | | | attachment | |
| 100963_at | 41 | | 2810403H05Rik | RIKEN cDNA 2810403H05 gene | — | — |
| 102049_at | 41 | 7305375 | Pdk4 | pyruvate dehydrogenase kinase, | IPR005467 // | HATPase_c // |
| | | | | isoenzyme 4 | Histidine kinase /// | Histidine kinase-, DNA |
| | | | | | IPR004358 // | gyrase B-, and |
| | | | | | Bacterial sensor | HSP90; 5e−19 |
| | | | | | protein C-terminal /// | |
| | | | | | IPR003594 // ATP- | |
| | | | | | binding protein, | |
| | | | | | ATPase-like | |
| 103319_at | 41 | | Psmd10 | proteasome (prosome, macropain) | IPR002110 // Ankyrin | ank // Ankyrin |
| | | | | 26S subunit, non-ATPase, 10 | | repeat; 8.1e−49 |
| 93040_at | 41 | | Fxyd1 | FXYD domain-containing ion | IPR000272 // | ATP1G1_PLM_MAT8 |
| | | | | transport regulator 1 | ATP1G1/PLM/MAT8 | // ATP1G1/PLM/MAT8 |
| | | | | | family | family; 4e−35 |
| 93948_at | 41 | | Nck2 | non-catalytic region of tyrosine | IPR001452 // SH3 | SH3 // SH3 |
| | | | | kinase adaptor protein 2 | domain /// IPR000980 | domain; 1.4e−57 /// |
| | | | | | // SH2 motif | SH2 // SH2 |
| | | | | | | domain; 6e−29 |
| 96388_at | 41 | | — | EST | — | — |
| 98924_at | 41 | | 4930569O04Rik | RIKEN cDNA 4930569O04 gene | IPR000768 // | — |
| | | | | | NAD:arginine ADP- | |
| | | | | | ribosyltransferase, | |
| | | | | | ART | |
| 100099_at | 40 | | Smpd1 | sphingomyelin phosphodiesterase | IPR000004 // | Metallophos // |
| | | | | 1, acid lysosomal | Saposin type B /// | Calcineurin-like |
| | | | | | IPR004843 // Metallo- | phosphoesterase; 6.9e−17 |
| | | | | | phosphoesterase | |
| 100756_r_at | 40 | | Tyms-ps | thymidylate synthase, pseudogene | — | — |
| 95149_at | 40 | | Copz1 | coatomer protein complex, subunit | IPR000804 // Clathrin | Clat_adaptor_s // |
| | | | | zeta 1 | adaptor complex, | Clathrin adaptor |
| | | | | | small chain | complex small |
| | | | | | | chain; 3.8e−76 |
| 95695_at | 40 | | — | — | — | — |
| 95721_at | 40 | | Mapkapk2 | MAP kinase-activated protein | IPR002290 // | — |
| | | | | kinase 2 | Serine/Threonine | |
| | | | | | protein kinase /// | |
| | | | | | IPR000719 // | |
| | | | | | Eukaryotic protein | |
| | | | | | kinase | |
| 99661_r_at | 40 | 6680991 | Cox7c | cytochrome c oxidase, subunit VIIc | IPR004202 // | COX7C // Cytochrome |
| | | | | | Cytochrome c | c oxidase subunit |
| | | | | | oxidase subunit VIIc | VIIc; 4e−33 |
| 100991_at | 39 | | Itgb1bp1 | integrin beta 1 binding protein 1 | IPR006020 // | — |
| | | | | | Phosphotyrosine | |
| | | | | | interaction domain | |
| 93786_i_at | 39 | | 1010001C05Rik | RIKEN cDNA 1010001C05 gene | — | — |
| 95468_at | 39 | | Egln1 | EGL nine homolog 1 (C. elegans) | IPR002893 // Zn- | 2OG-Fell_Oxy // 2OG- |
| | | | | | finger, MYND type /// | Fe(II) oxygenase |
| | | | | | IPR005123 // 2OG- | superfamily; 3.4e−10 |
| | | | | | Fe(II) oxygenase | |
| | | | | | superfamily | |
| 103492_at | 38 | | Cpxm1 | carboxypeptidase X 1 (M14 | IPR000834 // Zinc | F5_F8_type_C // F5/8 |
| | | | | family) | carboxypeptidase A | type C domain; 3.2e−70 |
| | | | | | metalloprotease | /// Zn_carbOpept // |
| | | | | | (M14) /// IPR000421 | Zinc |
| | | | | | // Coagulation factor | carboxypeptidase; 8.8e−21 |
| | | | | | 5/8 type C domain | |
| | | | | | (FA58C) /// | |
| | | | | | IPR001993 // | |
| | | | | | Mitochondrial | |
| | | | | | substrate carrier | |
| 95653_at | 38 | | Mrpl37 | mitochondrial ribosomal protein | — | — |
| | | | | L37 | | |
| 95718_f_at | 38 | 13128954 | Usmg5 | upregulated during skeletal muscle | — | — |
| | | | | growth 5 | | |
| 98545_at | 38 | 6671622 | Bcap37 | B-cell receptor-associated protein | IPR001107 // Band 7 | Band_7 // SPFH |
| | | | | 37 | protein /// IPR000163 | domain/Band 7 |
| | | | | | // Prohibitin | family; 6e−56 |
| 98616_f_at | 38 | | Myh7 | myosin, heavy polypeptide 7, | IPR004009 // Myosin | myosin_head // |
| | | | | cardiac muscle, beta | N-terminal SH3-like | Myosin head (motor |
| | | | | | domain /// IPR000048 | domain); 0 /// |
| | | | | | // IQ calmodulin- | Myosin_N // Myosin N- |
| | | | | | binding region /// | terminal SH3-like |
| | | | | | IPR002928 // Myosin | domain; 4.3e−18 /// IQ |
| | | | | | tail /// IPR001609 // | // IQ calmodulin- |
| | | | | | Myosin head (motor | binding motif; 0.01 /// |
| | | | | | domain) | Myosin_tail // Myosin |
| | | | | | | tail; 0 |
| 99678_f_at | 38 | 31980744 | Atp5l | ATP synthase, H+ transporting, | — | — |
| | | | | mitochondrial F0 complex, | | |
| | | | | subunit g | | |
| 100592_at | 37 | | Ghitm | growth hormone inducible | IPR002199 // Bax | — |
| | | | | transmembrane protein | inhibitor 1 | |
| 92845_at | 37 | 18266680 | Oxct | 3-oxoacid CoA transferase | IPR004165 // | CoA_trans // |
| | | | | | Coenzyme A | Coenzyme A |
| | | | | | transferase /// | transferase; 2.9e−197 |
| | | | | | IPR004164 // | |
| | | | | | Coenzyme A | |
| | | | | | transferase 2 /// | |
| | | | | | IPR004163 // | |
| | | | | | Coenzyme A | |
| | | | | | transferase 1 | |
| 93277_at | 37 | 31981679 | Hspd1 | heat shock protein 1 (chaperonin) | IPR002423 // | cpn60_TCP1 // TCP- |
| | | | | | Chaperonin | 1/cpn60 chaperonin |
| | | | | | Cpn60/TCP-1 /// | family; 2.3e−190 |
| | | | | | IPR001844 // | |
| | | | | | Chaperonin Cpn60 | |
| 93551_at | 37 | | 2510029B14Rik | RIKEN cDNA 2510029B14 gene | IPR000268 // RNA | — |
| | | | | | polymerases N/8 Kd | |
| | | | | | subunits | |
| 95076_at | 37 | | 1500032L24Rik | RIKEN cDNA 1500032L24 gene | — | — |
| 95426_at | 37 | 29789289 | Echs1 | enoyl Coenzyme A hydratase, | IPR001753 // Enoyl- | — |
| | | | | short chain, 1, mitochondrial | CoA | |
| | | | | | hydratase/isomerase | |
| 98561_at | 37 | | Tnni1 | troponin I, skeletal, slow 1 | IPR001978 // | Troponin // |
| | | | | | Troponin | Troponin; 2e−59 |
| 99536_at | 37 | | Kip2-pending | kinase interacting protein 2 | IPR002048 // | efhand // EF |
| | | | | | Calcium-binding EF- | hand; 3.4e−06 |
| | | | | | hand | |
| 102145_f_at | 36 | | Esrra | estrogen related receptor, alpha | IPR001628 // Zn- | zf-C4 // Zinc finger, C4 |
| | | | | | finger, C4-type | type (two |
| | | | | | steroid receptor /// | domains); 3.1e−51 /// |
| | | | | | IPR000324 // Vitamin | hormone_rec // |
| | | | | | D receptor /// | Ligand-binding domain |
| | | | | | IPR001723 // Steroid | of nuclear |
| | | | | | hormone receptor /// | hormone; 4.2e−32 |
| | | | | | IPR000536 // Ligand- | |
| | | | | | binding domain of | |
| | | | | | nuclear hormone | |
| | | | | | receptor /// | |
| | | | | | IPR000515 // | |
| | | | | | Binding-protein- | |
| | | | | | dependent transport | |
| | | | | | systems inner | |
| | | | | | membrane | |
| | | | | | component | |
| 93459_s_at | 36 | | Fzd4 | frizzled homolog 4 (Drosophila) | IPR000539 // Frizzled | Fz // Fz domain; 2.2e−65 |
| | | | | | protein /// IPR000024 | /// Frizzled // |
| | | | | | // Frizzled CRD | Frizzled/Smoothened |
| | | | | | region /// IPR000832 | family membrane |
| | | | | | // G-protein coupled | region; 1.7e−206 |
| | | | | | receptors family 2 | |
| | | | | | (secretin-like) | |
| 95693_at | 36 | | Idh2 | isocitrate dehydrogenase 2 | IPR001804 // | isodh // |
| | | | | (NADP+), mitochondrial | Isocitrate/isopropylmalate | Isocitrate/isopropylmalate |
| | | | | | dehydrogenase | dehydrogenase; 4.3e−116 |
| | | | | | /// IPR004790 // | /// isodh // |
| | | | | | Isocitrate | Isocitrate/isopropylmalate |
| | | | | | dehydrogenase | dehydrogenase; 1.1e−102 |
| | | | | | NADP-dependent, | |
| | | | | | eukaryotic | |
| 97279_at | 36 | 21704140 | AI265272 | EST AI265272 | IPR002204 // 3- | NAD_binding_2 // |
| | | | | | hydroxyisobutyrate | NAD binding domain |
| | | | | | dehydrogenase /// | of 6- |
| | | | | | IPR006115 // 6- | phosphogluconat; 0.0053 |
| | | | | | phosphogluconate | |
| | | | | | dehydrogenase, NAD | |
| | | | | | binding domain /// | |
| | | | | | IPR006183 // 6- | |
| | | | | | phosphogluconate | |
| | | | | | dehydrogenase | |
| 102378_at | 35 | | Sspn | sarcospan | — | — |
| 93114_at | 35 | 10181184 | Atp5j2 | ATP synthase, H+ transporting, | — | — |
| | | | | mitochondrial F0 complex, subunit | | |
| | | | | f, isoform 2 | | |
| 94375_at | 35 | | Hk2 | hexokinase 2 | IPR001312 // | hexokinase2 // |
| | | | | | Hexokinase | Hexokinase; 0 /// |
| | | | | | | hexokinase // |
| | | | | | | Hexokinase; 7.2e−290 |
| 100574_f_at | 34 | | Gpi1 | glucose phosphate isomerase 1 | IPR001672 // | PGI // |
| | | | | | Phosphoglucose | Phosphoglucose |
| | | | | | isomerase (PGI) | isomerase; 0 |
| 93740_at | 34 | | Nsep1 | nuclease sensitive element binding | IPR002059 // Cold- | CSD // ‘Cold-shock’ |
| | | | | protein 1 | shock DNA-binding | DNA-binding |
| | | | | | domain | domain; 4.7e−36 |
| 101347_at | 33 | | Igk-V8 | immunoglobulin kappa chain | IPR003600 // | — |
| | | | | variable 8 (V8) | Immunoglobulin-like | |
| | | | | | /// IPR003599 // | |
| | | | | | Immunoglobulin | |
| | | | | | subtype /// | |
| | | | | | IPR001865 // | |
| | | | | | Ribosomal protein S2 | |
| | | | | | /// IPR003006 // | |
| | | | | | Immunoglobulin/major | |
| | | | | | histocompatibility | |
| | | | | | complex /// | |
| | | | | | IPR003597 // | |
| | | | | | Immunoglobulin C- | |
| | | | | | type /// IPR003596 // | |
| | | | | | Immunoglobulin V- | |
| | | | | | type | |
| 101588_at | 33 | | Slc16a1 | solute carrier family 16 | IPR004743 // | — |
| | | | | (monocarboxylic acid | Monocarboxylate | |
| | | | | transporters), member 1 | transporter | |
| 101991_at | 33 | | Fmo1 | flavin containing monooxygenase 1 | IPR002253 // Flavin- | FMO-like // Flavin- |
| | | | | | containing | binding |
| | | | | | monooxygenase | monooxygenase-like; 0 |
| | | | | | (FMO) 1 /// | |
| | | | | | IPR001327 // FAD- | |
| | | | | | dependent pyridine | |
| | | | | | nucleotide-disulphide | |
| | | | | | oxidoreductase /// | |
| | | | | | IPR000759 // | |
| | | | | | Adrenodoxin | |
| | | | | | reductase /// | |
| | | | | | IPR000960 // Flavin- | |
| | | | | | containing | |
| | | | | | monooxygenase | |
| | | | | | FMO /// IPR000566 // | |
| | | | | | Lipocalin-related | |
| | | | | | protein and | |
| | | | | | Bos/Can/Equ | |
| | | | | | allergen | |
| 92646_at | 33 | | Mrpl23 | mitochondrial ribosomal protein | IPR001014 // | — |
| | | | | L23 | Ribosomal L23 | |
| | | | | | protein | |
| 93325_at | 33 | | Polr2e | polymerase (RNA) \|\| (DNA | — | — |
| | | | | directed) polypeptide E (25 kDa) | | |
| 94507_at | 33 | 31560705 | Facl2 | fatty acid Coenzyme A ligase, long | IPR000873 // AMP- | AMP-binding // AMP- |
| | | | | chain 2 | dependent | binding enzyme; 1.6e−103 |
| | | | | | synthetase and ligase | |
| 96122_at | 33 | | 2310016A09Rik | RIKEN cDNA 2310016A09 gene | IPR002925 // | — |
| | | | | | Dienelactone | |
| | | | | | hydrolase /// | |
| | | | | | IPR001064 // Beta | |
| | | | | | and gamma crystallin | |
| | | | | | /// IPR000379 // | |
| | | | | | Esterase/lipase/thioe | |
| | | | | | sterase, active site | |
| 96256_at | 33 | 6680690 | Prdx3 | peroxiredoxin 3 | IPR000866 // Alkyl | AhpC-TSA // |
| | | | | | hydroperoxide | AhpC/TSA |
| | | | | | reductase/Thiol | family; 3.1e−83 |
| | | | | | specific antioxidant/ | |
| | | | | | Mal allergen | |
| 96678_at | 33 | 13507612 | D14Ucla2 | DNA segment, Chr 14, University | IPR001092 // Basic | adh_short // short |
| | | | | of California at Los Angeles 2 | helix-loop-helix | chain |
| | | | | | dimerization domain | dehydrogenase; 1.9e−12 |
| | | | | | bHLH /// IPR002198 | |
| | | | | | // Short-chain | |
| | | | | | dehydrogenase/reductase | |
| | | | | | SDR /// | |
| | | | | | IPR002347 // | |
| | | | | | Glucose/ribitol | |
| | | | | | dehydrogenase | |
| 100538_at | 32 | | Sod1 | superoxide dismutase 1, soluble | IPR001424 // | — |
| | | | | | Copper/Zinc | |
| | | | | | superoxide | |
| | | | | | dismutase | |
| 101990_at | 32 | | Ldh2 | lactate dehydrogenase 2, B chain | IPR001236 // | ldh // lactate/malate |
| | | | | | Lactate/malate | dehydrogenase, NAD |
| | | | | | dehydrogenase /// | binding do; 2.6e−81 /// |
| | | | | | IPR001557 // L- | ldh_C // lactate/malate |
| | | | | | lactate | dehydrogenase, |
| | | | | | dehydrogenase | alpha/beta C-t; 3.3e−85 |
| 102302_at | 32 | | Bckdhb | branched chain ketoacid | — | — |
| | | | | dehydrogenase E1, beta | | |
| | | | | polypeptide | | |
| 93589_at | 32 | | Lysal1 | lysosomal apyrase-like 1 | IPR000407 // | GDA1_CD39 // |
| | | | | | GDA1/CD39 family of | GDA1/CD39 |
| | | | | | nucleoside | (nucleoside |
| | | | | | phosphatase | phosphatase) |
| | | | | | | family; 2.2e−93 |
| 101541_at | 31 | | — | ESTs, Weakly similar to S50828 | — | — |
| | | | | hypothetical protein —Escherichia | | |
| | | | | coil [E. coil] | | |
| 101580_at | 31 | 13384754 | Cox7b | cytochrome c oxidase subunit VIIb | — | — |
| 102128_f_at | 31 | 31981257 | Mrps25 | mitochondrial ribosomal protein | — | — |
| | | | | S25 | | |
| 92333_at | 31 | | Sirt1 | sirtuin 1 ((silent mating type | IPR003000 // Silent | SIR2 // Sir2 |
| | | | | information regulation 2, homolog) | information regulator | family; 1.7e−99 |
| | | | | 1 (S. cerevisiae) | protein Sir2 | |
| 94489_at | 31 | | Ptp4a1 | protein tyrosine phosphatase 4a1 | IPR000387 // | Y_phosphatase // |
| | | | | | Tyrosine specific | Protein-tyrosine |
| | | | | | protein phosphatase | phosphatase; 4.2e−07 |
| | | | | | and dual specificity | |
| | | | | | protein phosphatase | |
| | | | | | /// IPR000242 // | |
| | | | | | Tyrosine specific | |
| | | | | | protein phosphatase | |
| | | | | | /// IPR001230 // | |
| | | | | | Prenyl group binding | |
| | | | | | site (CAAX box) /// | |
| | | | | | IPR000340 // Dual | |
| | | | | | specificity protein | |
| | | | | | phosphatase | |
| 95016_at | 31 | | Nrp | neuropilin | IPR000998 // MAM | F5_F8_type_C // F5/8 |
| | | | | | domain /// IPR000421 | type C domain; 1.5e−128 |
| | | | | | // Coagulation factor | /// CUB // CUB |
| | | | | | 5/8 type C domain | domain; 9.7e−93 /// |
| | | | | | (FA58C) /// | MAM // MAM |
| | | | | | IPR000859 // CUB | domain; 1.6e−69 |
| | | | | | domain | |
| 99009_at | 31 | 31543330 | Nnt | nicotinamide nucleotide | IPR004003 // NAD(P) | PNTB // NAD(P) |
| | | | | transhydrogenase | transhydrogenase | transhydrogenase |
| | | | | | beta subunit /// | beta subunit; 0 /// |
| | | | | | IPR004571 // NAD(P) | AlaDh_PNT // Alanine |
| | | | | | transhydrogenase, | dehydrogenase/pyridine |
| | | | | | alpha subunit /// | nucleotide t; 1.1e−74 |
| | | | | | IPR004002 // Alanine | |
| | | | | | dehydrogenase and | |
| | | | | | pyridine nucleotide | |
| | | | | | transhydrogenase | |
| 102402_at | 30 | 6679957 | Gbas | glioblastoma amplified sequence | — | — |
| 92371_at | 30 | | Hrc | histidine rich calcium binding | IPR000561 // EGF- | — |
| | | | | protein | like domain /// | |
| | | | | | IPR002049 // | |
| | | | | | Laminin-type EGF- | |
| | | | | | like domain | |
| 93308_s_at | 30 | 6679237 | Pcx | pyruvate carboxylase | IPR005482 // Biotin | HMGL-like // HMGL- |
| | | | | | carboxylase, C- | like; 3.5e−43 /// |
| | | | | | terminal /// | biotin_lipoyl // Biotin- |
| | | | | | IPR005930 // | requiring |
| | | | | | Pyruvate carboxylase | enzyme; 1.7e−26 /// |
| | | | | | /// IPR005481 // | CPSase_L_D2 // |
| | | | | | Carbamoyl- | Carbamoyl-phosphate |
| | | | | | phosphate | synthase L |
| | | | | | synthetase large | chain,; 1.7e−100 /// |
| | | | | | chain, N-terminal /// | Biotin_carb_C // Biotin |
| | | | | | IPR003379 // | carboxylase C- |
| | | | | | Conserved | terminal domain; 2.3e−61 |
| | | | | | carboxylase region /// | /// |
| | | | | | IPR001882 // Biotin- | CPSase_L_chain // |
| | | | | | requiring enzyme, | Carbamoyl-phosphate |
| | | | | | attachment site /// | synthase L |
| | | | | | IPR000089 // | chain,; 2.4e−43 /// |
| | | | | | Biotin/lipoyl | PYC_OADA // |
| | | | | | attachment /// | Conserved |
| | | | | | IPR005479 // | carboxylase |
| | | | | | Carbamoyl- | domain; 4.4e−121 |
| | | | | | phosphate synthase | |
| | | | | | L chain, ATP-binding | |
| | | | | | /// IPR000891 // | |
| | | | | | HMG-CoA lyase-like | |
| 94668_at | 30 | | — | ESTs | — | — |
| 95067_at | 30 | | Mrpl2 | mitochondrial ribosomal protein L2 | IPR002171 // | Ribosomal_L2_C // |
| | | | | | Ribosomal protein L2 | Ribosomal Proteins |
| | | | | | | L2, C-terminal |
| | | | | | | doma; 4.6e−46 /// |
| | | | | | | Ribosomal_L2 // |
| | | | | | | Ribosomal Proteins |
| | | | | | | L2, RNA binding |
| | | | | | | dom; 9.2e−29 |
| 97410_at | 30 | | D130005A03 | hypothetical protein D130005A03 | — | — |
| 98610_at | 30 | | 1500012D08Rik | RIKEN cDNA 1500012D08 gene | IPR003029 // RNA | — |
| | | | | | binding S1 | |
| 99507_at | 30 | | Ucp1 | uncoupling protein 1, | IPR002030 // | mito_carr // |
| | | | | mitochondrial | Mitochondrial brown | Mitochondrial carrier |
| | | | | | fat uncoupling protein | protein; 2e−79 |
| | | | | | /// IPR002113 // | |
| | | | | | Adenine nucleotide | |
| | | | | | translocator 1 /// | |
| | | | | | IPR001993 // | |
| | | | | | Mitochondrial | |
| | | | | | substrate carrier | |
| AFFX- | 30 | 6679937 | | | | |
| GapdhMur/ | | | | | | |
| M32599_3_at | | | | | | |
| 100671_at | 29 | | — | — | — | — |
| 102668_at | 29 | | Ppara | peroxisome proliferator activated | IPR001628 // Zn- | zf-C4 // Zinc finger, C4 |
| | | | | receptor alpha | finger, C4-type | type (two |
| | | | | | steroid receptor /// | domains); 1.1e−46 /// |
| | | | | | IPR003074 // | hormone_rec // |
| | | | | | Peroxisome | Ligand-binding domain |
| | | | | | proliferator-activated | of nuclear |
| | | | | | receptor /// | hormone; 3.1e−38 |
| | | | | | IPR001723 // Steroid | |
| | | | | | hormone receptor /// | |
| | | | | | IPR003076 // | |
| | | | | | Peroxisome | |
| | | | | | proliferator-activated | |
| | | | | | receptor, alpha /// | |
| | | | | | IPR000536 // Ligand- | |
| | | | | | binding domain of | |
| | | | | | nuclear hormone | |
| | | | | | receptor | |
| 103881_at | 29 | 22203753 | 1110013G13Rik | RIKEN cDNA 1110013G13 gene | IPR001596 // | Pyrophosphatase // |
| | | | | | Inorganic | Inorganic |
| | | | | | pyrophosphatase | pyrophosphatase; 1.1e−107 |
| 104577_at | 29 | | Mlh1 | mutL homolog 1 (E. coli) | IPR002099 // DNA | DNA_mis_repair // |
| | | | | | mismatch repair | DNA mismatch repair |
| | | | | | protein /// IPR003594 | protein, C- |
| | | | | | // ATP-binding | termina; 1.7e−43 /// |
| | | | | | protein, ATPase-like | HATPase_c // |
| | | | | | | Histidine kinase-, DNA |
| | | | | | | gyrase B-, |
| | | | | | | and; 0.00044 |
| 92592_at | 29 | | Gpd1 | glycerol-3-phosphate | IPR0006109 // NAD- | NAD_Gly3P_dh // |
| | | | | dehydrogenase 1 (soluble) | dependent glycerol-3- | NAD-dependent |
| | | | | | phosphate | glycerol-3-phosphate |
| | | | | | dehydrogenase | dehyd; 5.8e−204 |
| | | | | | domain /// IPR006168 | |
| | | | | | // NAD-dependent | |
| | | | | | glycerol-3-phosphate | |
| | | | | | dehydrogenase | |
| 93050_at | 29 | | Mylpc | myosin light chain, | IPR002048 // | efhand // EF |
| | | | | phosphorylatable, cardiac | Calcium-binding EF- | hand; 1.7e−12 |
| | | | | ventricles | hand | |
| 93646_at | 29 | | Ptk9 | PTK9 protein tyrosine kinase 9 | IPR002108 // Actin- | cofilin_ADF // |
| | | | | | binding, | Cofilin/tropomyosin- |
| | | | | | cofilin/tropomyosin | type actin-binding |
| 94902_at | 29 | | Sod3 | superoxide dismutase 3, | type | pr; 3.8e−08 |
| | | | | extracellular | IPR001424 // | sodcu // Copper/zinc |
| | | | | | Copper/Zinc | superoxide dismutase |
| | | | | | superoxide | (SODC); 1e−67 |
| | | | | | dismutase | |
| 96856_at | 29 | 6680816 | C1qbp | complement component 1, q | IPR003428 // | MAM33 // |
| | | | | subcomponent binding protein | Mitochondrial | Mitochondrial |
| | | | | | glycoprotein | glycoprotein; 2e−108 |
| 98056_at | 29 | | Phlda3 | pleckstrin homology-like domain, | IPR001849 // | — |
| | | | | family A, member 3 | Pleckstrin-like | |
| 98876_at | 29 | | Mrpl11 | mitochondrial ribosomal protein | IPR000911 // | Ribosomal_L11 // |
| | | | | L11 | Ribosomal protein | Ribosomal protein |
| | | | | | L11 | L11, RNA binding |
| | | | | | | do; 3.7e−18 /// |
| | | | | | | Ribosomal_L11_N // |
| | | | | | | Ribosomal protein |
| | | | | | | L11, N-terminal |
| | | | | | | dom; 7.1e−25 |
| 99604_at | 29 | | 1810015H18Rik | RIKEN cDNA 1810015H18 gene | — | — |
| 99667_at | 29 | 6753502 | Cox6a2 | cytochrome c oxidase, subunit VIa, | IPR001349 // | COX6A // Cytochrome |
| | | | | polypeptide 2 | Cytochrome c | c oxidase subunit |
| | | | | | oxidase, subunit VIa | VIa; 1.9e−51 |
| AFFX- | 29 | 6679937 | | | | |
| GapdhMur/ | | | | | | |
| M32599_5_st | | | | | | |
| AFEX- | 29 | 6679237 | | | | |
| PyruCarbMur/ | | | | | | |
| L09192_MA_at | | | | | | |
| 101063_at | 28 | | Tncc | troponin C, cardiac/slow skeletal | IPR002048 // | efhand // EF |
| | | | | | Calcium-binding EF- | hand; 1.5e−25 |
| | | | | | hand /// IPR001125 // | |
| | | | | | Recoverin | |
| 92553_at | 28 | | Es10 | esterase 10 | IPR000801 // | Esterase // Putative |
| | | | | | Putative esterase /// | esterase; 5.5e−107 |
| | | | | | IPR000379 // | |
| | | | | | Esterase/lipase/thioesterase, | |
| | | | | | active site | |
| 93514_at | 28 | | — | — | — | — |
| 94166_g_at | 28 | | Ccl1 | chemokine (C-C motif) ligand 1 | IPR001811 // Small | IL8 // Small cytokines |
| | | | | | chemokine, | (intecrine/chemokine), |
| | | | | | interleukin-8 like /// | inter; 2.2e−23 |
| | | | | | IPR000827 // Small | |
| | | | | | chemokine, C-C | |
| | | | | | subfamily | |
| 96003_at | 28 | | Mta1l1 | metastasis associated 1-like 1 | IPR001005 // Myb | myb_DNA-binding // |
| | | | | | DNA-binding domain | Myb-like DNA-binding |
| | | | | | /// IPR000949 // | domain; 3.2e−09 /// |
| | | | | | ELM2 domain /// | ELM2 // ELM2 |
| | | | | | IPR000679 // Zn- | domain; 1.4e−21 /// |
| | | | | | finger, GATA type /// | BAH // BAH |
| | | | | | IPR000345 // | domain; 5.7e−20 /// |
| | | | | | Cytochrome c heme- | GATA // GATA zinc |
| | | | | | binding site /// | finger; 2.9e−14 |
| | | | | | IPR001025 // Bromo | |
| | | | | | adjacent region | |
| 97265_at | 28 | | 1810013D10Rik | RIKEN cDNA 1810013D10 gene | — | — |
| 97319_at | 28 | | Rrad | Ras-related associated with | IPR003575 // Ras | ras // Ras family; 1.8e−16 |
| | | | | diabetes | small GTPase /// | |
| | | | | | IPR005225 // Small | |
| | | | | | GTP-binding protein | |
| | | | | | domain /// IPR001806 | |
| | | | | | // Ras GTPase | |
| | | | | | superfamily | |
| 97951_s_at | 28 | | Tsc2 | tuberous sclerosis 2 | IPR003913 // Tuberin | Tuberin // Tuberin; 0 /// |
| | | | | | /// IPR000331 // | Rap_GAP // Rap/ran- |
| | | | | | Rap/ran-GAP | GAP; 2.4e−84 |
| 98039_at | 28 | | 2410015M20Rik | RIKEN cDNA 2410015M20 gene | — | — |
| 99532_at | 28 | | Tob1 | transducer of ErbB-2.1 | — | Anti_proliferat // BTG1 |
| | | | | | | family; 3.1e−100 |
| 100535_at | 27 | | Eif4g2 | eukaryotic translation initiation | IPR000504 // RNA- | W2 // eIF4- |
| | | | | factor 4, gamma 2 | binding region RNP-1 | gamma/eIF5/eIF2- |
| | | | | | (RNA recognition | epsilon; 7.1e−33 /// |
| | | | | | motif) /// IPR003890 | MA3 // MA3 |
| | | | | | // Initiation factor eIF- | domain; 4.5e−33 /// |
| | | | | | 4 gamma, middle /// | MIF4G // MIF4G |
| | | | | | IPR003891 // | domain; 2.7e−61 |
| | | | | | Initiation factor eIF-4 | |
| | | | | | gamma, MA3 /// | |
| | | | | | IPR003307 // eIF4- | |
| | | | | | gamma/eIF5/eIF2- | |
| | | | | | epsilon | |
| 101028_i_at | 27 | | Actc1 | actin, alpha, cardiac | IPR004000 // | actin // Actin; 1.2e−276 |
| | | | | | Actin/actin-like /// | |
| | | | | | IPR004001 // Actin | |
| 101409_at | 27 | | Lgtn | ligatin | IPR004521 // | — |
| | | | | | Uncharacterized | |
| | | | | | domain 2 /// | |
| | | | | | IPR001950 // | |
| | | | | | Translation initiation | |
| | | | | | factor SUI1 /// | |
| | | | | | IPR002478 // PUA | |
| | | | | | domain | |
| 101946_at | 27 | 6678760 | Lypla1 | lysophospholipase 1 | IPR003140 // | abhydrolase_2 // |
| | | | | | Phospholipase/ | Phospholipase/ |
| | | | | | Carboxylesterase /// | Carboxylesterase; |
| | | | | | IPR000379 // | 2.2e−121 |
| | | | | | Esterase/lipase/thioesterase, | |
| | | | | | active site | |
| 102560_at | 27 | | — | — | — | — |
| 103559_at | 27 | | Prkaca | protein kinase, cAMP dependent, | IPR000961 // Protein | pkinase_C // Protein |
| | | | | catalytic, alpha | kinase-C-terminal | kinase C terminal |
| | | | | | domain /// IPR002290 | domain; 0.00063 /// |
| | | | | | // Serine/Threonine | pkinase // Protein |
| | | | | | protein kinase /// | kinase domain; 1.5e−84 |
| | | | | | IPR000719 // | |
| | | | | | Eukaryotic protein | |
| | | | | | kinase | |
| 92831_at | 27 | | Sfxn1 | sideroflexin 1 | IPR004686 // | Mtc // Tricarboxylate |
| | | | | | Tricarboxylate/iron | carrier; 2e−200 |
| | | | | | carrier | |
| 93196_at | 27 | | D8Ertd531e | DNA segment, Chr 8, ERATO Doi | — | — |
| | | | | 531, expressed | | |
| 94192_at | 27 | | Gdap10 | ganglioside-induced | — | — |
| | | | | differentiation- | | |
| | | | | associated-protein 10 | | |
| 94381_at | 27 | | Umpk | uridine monophosphate kinase | IPR000764 // Uridine | — |
| | | | | | kinase /// IPR006083 | |
| | | | | | // | |
| | | | | | Phosphoribulokinase/ | |
| | | | | | uridine kinase | |
| 94925_at | 27 | | 1810055D05Rik | RIKEN cDNA 1810055D05 gene | IPR001623 // Heat | DnaJ // DnaJ |
| | | | | | shock protein DnaJ, | domain; 2.3e−05 |
| | | | | | N-terminal | |
| 95469_at | 27 | | Btd | biotinidase | IPR003010 // | CN_hydrolase // |
| | | | | | Nitrilase/cyanide | Carbon-nitrogen |
| | | | | | hydratase | hydrolase; 2.4e−05 |
| 95587_at | 27 | | — | Mus musculus adult male adrenal | — | — |
| | | | | gland cDNA, RIKEN full-length | | |
| | | | | enriched library, | | |
| | | | | clone: B330005C17 | | |
| | | | | product: hypothetical Arginine-rich | | |
| | | | | region containing protein, | | |
| | | | | full insert sequence. | | |
| 95869_at | 27 | | — | ESTs | — | — |
| 95943_at | 27 | | — | ESTs | — | — |
| 96243_f_at | 27 | | Aldh9a1 | aldehyde dehydrogenase 9, | IPR002086 // | aldedh // Aldehyde |
| | | | | subfamily A1 | Aldehyde | dehydrogenase |
| | | | | | dehydrogenase | family; 3.9e−212 |
| 96348_at | 27 | | 0610039C21Rik | RIKEN cDNA 0610039C21 gene | IPR002641 // Patatin | Patatin // Patatin-like |
| | | | | | | phospholipase; 7.7e−34 |
| 96355_at | 27 | | 2900055D03Rik | RIKEN cDNA 2900055003 gene | — | — |
| 97777_at | 27 | | Nkx2-5 | NK2 transcription factor related, | IPR001356 // | homeobox // |
| | | | | locus 5 (Drosophila) | Homeobox | Homeobox |
| | | | | | | domain; 8.9e−27 |
| 99331_at | 27 | | Apeg1 | aortic preferentially expressed | IPR003006 // | ig // Immunoglobulin |
| | | | | gene 1 | Immunoglobulin/major | domain; 0.00073 |
| | | | | | histocompatibility | |
| | | | | | complex /// | |
| | | | | | IPR002290 // | |
| | | | | | Serine/Threonine | |
| | | | | | protein kinase /// | |
| | | | | | IPR003599 // | |
| | | | | | Immunoglobulin | |
| | | | | | subtype /// | |
| | | | | | IPR003600 // | |
| | | | | | Immunoglobulin-like | |
| | | | | | /// IPR003961 // | |
| | | | | | Fibronectin, type III /// | |
| | | | | | IPR001245 // | |
| | | | | | Tyrosine protein | |
| | | | | | kinase /// IPR002965 | |
| | | | | | // Praline-rich | |
| | | | | | extensin /// | |
| | | | | | IPR000719 // | |
| | | | | | Eukaryotic protein | |
| | | | | | kinase /// IPR003598 | |
| | | | | | // Immunoglobulin C- | |
| | | | | | 2 type | |
| 99994_at | 27 | | Cidea | cell death-inducing DNA | IPR003508 // | CIDE-N // CIDE-N |
| | | | | fragmentation factor, alpha | Caspase-activated | domain; 7.7e−51 |
| | | | | subunit-like effector A | nuclease CIDE-N | |
| 100614_at | 26 | | Mb | myoglobin | — | globin // Globin; 1.4e−36 |
| 100921_at | 26 | | Tnni3 | troponin I, cardiac | IPR001978 // | Troponin // |
| | | | | | Troponin | Troponin; 7.3e−59 |
| 101015_s_at | 26 | | Ifnar2 | interferon (alpha and beta) | IPR000282 // | — |
| | | | | receptor 2 | Cytokine receptor | |
| | | | | | class 2 | |
| 101490_at | 26 | | 1810010A06Rik | RIKEN cDNA 1810010A06 gene | IPR000361 // Protein | HesB-like // HesB-like |
| | | | | | of unknown function, | domain; 4e−42 |
| | | | | | HesB/YadR/YfhF | |
| 102653_at | 26 | | Ryr2 | ryanodine receptor 2, cardiac | IPR005821 // Ion | RyR // RyR |
| | | | | | transport protein /// | domain; 8.8e−227 /// |
| | | | | | IPR003877 // | MIR // MIR |
| | | | | | SPla/RYanodine | domain; 3.1e−40 /// |
| | | | | | receptor SPRY /// | SPRY // SPRY |
| | | | | | IPR003608 // MIR | domain; 6.9e−116 /// |
| | | | | | domain /// IPR002048 | RYDR_ITPR // RIH |
| | | | | | // Calcium-binding | domain; 1.4e−179 /// |
| | | | | | EF-hand /// | ion_trans // Ion |
| | | | | | IPR000699 // | transport protein; 2.1e−05 |
| | | | | | Intracellular calcium- | /// efhand // EF |
| | | | | | release channel /// | hand; 0.0053 |
| | | | | | IPR003032 // | |
| | | | | | Ryanodine receptor | |
| | | | | | Ryr /// IPR001215 // | |
| | | | | | Ryanodine receptor | |
| | | | | | /// IPR001682 // | |
| | | | | | Ca2+/Na+ channel, | |
| | | | | | pore region | |
| 103939_at | 26 | | 2610509I15Rik | RIKEN cDNA 2610509I15 gene | IPR001753 // Enoyl- | ECH // Enoyl-CoA |
| | | | | | CoA | hydratase/isomerase |
| | | | | | hydratase/isomerase | family; 6.2e−20 |
| 104325_at | 26 | | 1110025G12Rik | RIKEN cDNA 1110025G12 gene | — | — |
| 104743_at | 26 | | Cdh13 | cadherin 13 | IPR002126 // | cadherin // Cadherin |
| | | | | | Cadherin | domain; 1e−114 |
| 94554_at | 26 | | 4021401A16Rik | RIKEN cDNA 4021401A16 gene | — | TRAPP_Bet3 // |
| | | | | | | Transport protein |
| | | | | | | particle (TRAPP) |
| | | | | | | compone; 2.3e−123 |
| 96089_at | 26 | | 4931406C07Rik | RIKEN cDNA 4931406C07 gene | — | — |
| 96237_at | 26 | | SMAF1 | SMAF1 | — | — |
| 97248_at | 26 | 6681137 | Dbi | diazepam binding inhibitor | IPR000582 // Acyl- | ACBP // Acyl CoA |
| | | | | | coA-binding protein, | binding protein; 1.8e−52 |
| | | | | | ACBP | |
| 97430_at | 26 | | G6pt1 | glucose-6-phosphatase, transport | IPR000849 // GlpT | sugar_tr // Sugar (and |
| | | | | protein 1 | family of transporters | other) |
| | | | | | /// IPR005828 // | transporter; 0.00018 |
| | | | | | General substrate | |
| | | | | | transporter | |
| 98984_f_at | 26 | 31981769 | Gpd2 | glycerol phosphate | IPR002048 // | efhand // EF |
| | | | | dehydrogenase 1, mitochondrial | Calcium-binding EF- | hand; 9.4e−09 /// DAO |
| | | | | | hand /// IPR006076 // | // FAD dependent |
| | | | | | FAD dependent | oxidoreductase; 3.6e−158 |
| | | | | | oxidoreductase /// | |
| | | | | | IPR000447 // FAD- | |
| | | | | | dependent glycerol-3- | |
| | | | | | phosphate | |
| | | | | | dehydrogenase | |
| 99154_s_at | 26 | | — | Mus musculus, Similar to PTD015 | — | — |
| | | | | protein, clone MGC: 36240 | | |
| | | | | IMAGE: 5027461, mRNA, | | |
| | | | | complete cds | | |
| 99570_s_at | 26 | | Atp2a2 | ATPase, Ca++ transporting, | IPR004014 // Cation | Cation_ATPase_N // |
| | | | | cardiac muscle, slow twitch 2 | transporting ATPase, | Cation |
| | | | | | N terminal /// | transporter/ATPase, |
| | | | | | IPR001757 // | N-terminus; 2.2e−26 /// |
| | | | | | ATPase, E1-E2 type | E1-E2_ATPase // E1- |
| | | | | | /// IPR006069 // | E2 ATPase; 2.5e−123 |
| | | | | | Cation transporting | /// Cation_ATPase_C |
| | | | | | ATPase /// | // Cation transporting |
| | | | | | IPR005834 // | ATPase, C- |
| | | | | | haloacid | terminu; 6.5e−84 /// |
| | | | | | dehalogenase-like | Hydrolase // haloacid |
| | | | | | hydrolase /// | dehalogenase-like |
| | | | | | IPR006068 // Cation | hydrolase; 6.1e−12 |
| | | | | | transporting ATPase, | |
| | | | | | C-terminal /// | |
| | | | | | IPR000695 // H+ | |
| | | | | | transporting ATPase, | |
| | | | | | proton pump | |
| 100400_at | 25 | | 4921531G14Rik | RIKEN cDNA 4921531G14 gene | IPR001440 // TPR | TPR // TPR |
| | | | | | repeat | Domain; 0.005 |
| 100726_at | 25 | | Grin2a | glutamate receptor, ionotropic, | IPR001311 // Solute- | lig_chan // Ligand- |
| | | | | NMDA2A (epsilon 1) | binding | gated ion |
| | | | | | protein/glutamate | channel; 4.4e−107 |
| | | | | | receptor /// | |
| | | | | | IPR001508 // NMDA | |
| | | | | | receptor /// | |
| | | | | | IPR001320 // | |
| | | | | | Ionotropic glutamate | |
| | | | | | receptor | |
| 101071_at | 25 | | Myhca | myosin heavy chain, cardiac | IPR004009 // Myosin | myosin_head // |
| | | | | muscle, adult | N-terminal SH3-like | Myosin head (motor |
| | | | | | domain /// IPR000048 | domain); 0 /// |
| | | | | | // IQ calmodulin- | Myosin_N // Myosin N- |
| | | | | | binding region /// | terminal SH3-like |
| | | | | | IPR002928 // Myosin | domain; 2.5e−17 /// IQ |
| | | | | | tail /// IPR000533 // | // IQ calmodulin- |
| | | | | | Tropomyosin /// | binding motif; 0.0029 |
| | | | | | IPR001609 // Myosin | /// Myosin_tail // |
| | | | | | head (motor domain) | Myosin tail; 0 |
| 101082_at | 25 | | Mod1 | malic enzyme, supernatant | IPR001891 // Malic | malic_N // Malic |
| | | | | | oxidoreductase | enzyme, NAD binding |
| | | | | | | domain; 8.6e−126 /// |
| | | | | | | malic // Malic enzyme, |
| | | | | | | N-terminal |
| | | | | | | domain; 1.1e−123 |
| 101605_at | 25 | | — | ESTs | — | — |
| 101844_at | 25 | | Pso | peroxisomal sarcosine oxidase | — | — |
| 102314_at | 25 | | Slc2a4 | solute carrier family 2 (facilitated | IPR003663 // Sugar | sugar_tr // Sugar (and |
| | | | | glucose transporter), member 4 | transporter /// | other) |
| | | | | | IPR005829 // Sugar | transporter; 1.9e−185 |
| | | | | | transporter | |
| | | | | | superfamily /// | |
| | | | | | IPR005828 // General | |
| | | | | | substrate transporter | |
| | | | | | /// IPR000803 // | |
| | | | | | Facilitated glucose | |
| | | | | | transporter family /// | |
| | | | | | IPR002441 // | |
| | | | | | Glucose transporter, | |
| | | | | | type 4 (GLUT4) | |
| 103084_at | 25 | | Csrp3 | cysteine-rich protein 3 | IPR001781 // Zn- | LIM // LIM |
| | | | | | binding protein, LIM | domain; 1.4e−32 |
| 103422_at | 25 | | Cd1d1 | CD1d1 antigen | IPR003006 // | — |
| | | | | | Immunoglobulin/major | |
| | | | | | histocompatibility | |
| | | | | | complex /// | |
| | | | | | IPR003597 // | |
| | | | | | Immunoglobulin C- | |
| | | | | | type | |
| 103495_at | 25 | | — | ESTs | — | — |
| 104725_at | 25 | | Tc10-pending | ras-like protein | IPR003577 // Ras | ras // Ras family; 1.8e−79 |
| | | | | | small GTPase, Ras | |
| | | | | | type /// IPR003578 // | |
| | | | | | Ras small GTPase, | |
| | | | | | Rho type /// | |
| | | | | | IPR001230 // Prenyl | |
| | | | | | group binding site | |
| | | | | | (CAAX box) /// | |
| | | | | | IPR003579 // Ras | |
| | | | | | small GTPase, Rab | |
| | | | | | type /// IPR001806 // | |
| | | | | | Ras GTPase | |
| | | | | | superfamily | |
| 92241_at | 25 | | 1500041O16Rik | RIKEN cDNA 1500041O16 gene | — | — |
| 95908_at | 25 | | Klra1 | killer cell lectin-like receptor, | IPR001304 // C-type | lectin_c // Lectin C- |
| | | | | subfamily A, member 1 | lectin | type domain; 1.5e−09 |
| 96803_at | 25 | | Gbe1 | glucan (1,4-alpha-), branching | IPR004193 // | isoamylase_N // |
| | | | | enzyme 1 | Glycoside hydrolase, | Isoamylase N-terminal |
| | | | | | family 13, N-terminal | domain; 1e−27 /// |
| | | | | | /// IPR006047 // | alpha-amylase // |
| | | | | | Alpha amylase, | Alpha amylase, |
| | | | | | catalytic domain | catalytic domain; 4.3e−07 |
| 97207_f_at | 25 | 6678760 | Lypla1 | lysophospholipase 1 | IPR003140 // | abhydrolase_2 // |
| | | | | | Phospholipase/ | Phospholipase/ |
| | | | | | Carboxylesterase /// | Carboxylesterase; |
| | | | | | IPR000379 // | 2.2e−121 |
| | | | | | Esterase/lipase/thioesterase, | |
| | | | | | active site | |
| 97302_at | 25 | | Nd1-pending | Nd1 | IPR000210 // | Kelch // Kelch |
| | | | | | BTB/POZ domain /// | motif; 2.1e−98 /// BTB |
| | | | | | IPR001798 // Kelch | // BTB/POZ |
| | | | | | repeat | domain; 6.8e−28 |
| 98497_at | 25 | | Eps15-rs | epidermal growth factor receptor | IPR002048 // | efhand // EF |
| | | | | pathway substrate 15, related | Calcium-binding EF- | hand; 5.6e−15 |
| | | | | sequence | hand /// IPR000261 // | |
| | | | | | EPS15 homology | |
| | | | | | (EH) /// IPR005613 // | |
| | | | | | Actin interacting | |
| | | | | | protein 3 /// | |
| | | | | | IPR003903 // | |
| | | | | | Ubiquitin interacting | |
| | | | | | motif | |
| 99108_s_at | 25 | | — | — | — | — |
| 99631_f_at | 25 | 6680988 | Cox6a1 | cytochrome c oxidase, subunit VI | IPR001349 // | COX6A // Cytochrome |
| | | | | a, polypeptide 1 | Cytochrome c | c oxidase subunit |
| | | | | | oxidase, subunit VIa | VIa; 1.9e−53 |
| AFFX- | 25 | 6679937 | | | | |
| GapdhMur/ | | | | | | |
| M32599_M_at | | | | | | |
| 100136_at | 24 | | Lamp2 | lysosomal membrane | IPR002000 // | Lamp // Lysosome- |
| | | | | glycoprotein 2 | Lysosome-associated | associated membrane |
| | | | | | membrane | glycoprotein (L; 7.6e−241 |
| | | | | | glycoprotein | |
| | | | | | (Lamp)/CD68 /// | |
| | | | | | IPR001412 // | |
| | | | | | Aminoacyl-tRNA | |
| | | | | | synthetase, class I | |
| 100403_at | 24 | | Mylc2a | myosin light chain, regulatory A | — | efhand // EF |
| | | | | | | hand; 1.8e−08 |
| 100593_at | 24 | | Tnnt2 | troponin T2, cardiac | IPR001978 // | Troponin // |
| | | | | | Troponin | Troponin; 1.7e−38 |
| 101214_f_at | 24 | 6679937 | Gapd | glyceraldehyde-3-phosphate | IPR000173 // | gpdh // |
| | | | | dehydrogenase | Glyceraldehyde 3- | Glyceraldehyde 3- |
| | | | | | phosphate | phosphate |
| | | | | | dehydrogenase | dehydrogenase, |
| | | | | | | NA; 2.5e−102 /// |
| | | | | | | gpdh_C // |
| | | | | | | Glyceraldehyde 3- |
| | | | | | | phosphate |
| | | | | | | dehydrogenase, C-; |
| | | | | | | 1.3e−123 |
| 101532_g_at | 24 | | Aldo2 | aldolase 2, B isoform | IPR000741 // | glycolytic_enzy // |
| | | | | | Fructose- | Fructose- |
| | | | | | bisphosphate | bisphosphate aldolase |
| | | | | | aldolase, class-I | class-; 3.7e−243 |
| 101538_i_at | 24 | | Ces3 | carboxylesterase 3 | IPR002018 // | COesterase // |
| | | | | | Carboxylesterase, | Carboxylesterase; 2.5e−206 |
| | | | | | type B /// IPR000379 | |
| | | | | | // | |
| | | | | | Esterase/lipase/thioesterase, | |
| | | | | | active site | |
| 101676_at | 24 | | Gpx3 | glutathione peroxidase 3 | IPR000889 // | GSHPx // Glutathione |
| | | | | | Glutathione | peroxidase; 7.9e−68 |
| | | | | | peroxidase | |
| 102048_at | 24 | | Crap | cardiac responsive adriamycin | IPR002110 // Ankyrin | ank // Ankyrin |
| | | | | protein | | repeat; 2e−35 |
| 103255_at | 24 | | Traf5 | Tnf receptor-associated factor 5 | IPR003007 // Meprin | zf-TRAF // TRAF-type |
| | | | | | A, C-terminal TRAF | zinc finger; 1.1e−45 /// |
| | | | | | /// IPR001293 /// Zn- | MATH // MATH |
| | | | | | finger, TRAF type /// | domain; 2.7e−36 |
| | | | | | IPR001841 // Zn- | |
| | | | | | finger, RING /// | |
| | | | | | IPR000345 // | |
| | | | | | Cytochrome c heme- | |
| | | | | | binding site /// | |
| | | | | | IPR002083 // | |
| | | | | | Meprin/TRAF-like | |
| | | | | | MATH | |
| 103442_at | 24 | | LOC216820 | similar to DKFZP566O084 protein | IPR001986 // EPSP | adh_short // short |
| | | | | | synthase (3- | chain |
| | | | | | phosphoshikimate 1- | dehydrogenase; 1e−52 |
| | | | | | carboxyvinyltransferase) | |
| | | | | | /// IPR002198 // | |
| | | | | | Short-chain | |
| | | | | | dehydrogenase/reductase | |
| | | | | | SDR /// | |
| | | | | | IPR002347 // | |
| | | | | | Glucose/ribitol | |
| | | | | | dehydrogenase | |
| 103719_at | 24 | | Msh5 | mutS homolog 5 (E. coli) | IPR002863 // DNA | MutS_N // MutS |
| | | | | | mismatch repair | family, N-terminal |
| | | | | | protein MutS, N- | putative DNA |
| | | | | | terminal /// | binding; 0.00025 /// |
| | | | | | IPR000432 // DNA | MutS_C // DNA |
| | | | | | mismatch repair | mismatch repair |
| | | | | | protein MutS, C- | proteins, mutS |
| | | | | | terminal | family; 5.6e−55 |
| 103782_at | 24 | | Clcnk1 | chloride channel K1 | IPR000644 // CBS | voltage_CLC // |
| | | | | | domain /// IPR002250 | Voltage gated chloride |
| | | | | | // Chloride channel | channel; 5e−155 /// |
| | | | | | CLC-K /// IPR001807 | CBS // CBS |
| | | | | | // Cl-channel, voltage | domain; 4.3e−10 |
| | | | | | gated | |
| 104161_at | 24 | | Cpsf2 | cleavage and polyadenylation | — | — |
| | | | | specific factor 2 | | |
| 104338_r_at | 24 | | 1200008D14Rik | RIKEN cDNA 1200008D14 gene | IPR000225 // | Armadillo_seg // |
| | | | | | Armadillo repeat | Armadillo/beta- |
| | | | | | | catenin-like |
| | | | | | | repeat; 6.6e−36 |
| 104648_at | 24 | | Pacs1 | phosphofurin acidic cluster sorting | — | — |
| | | | | protein 1 | | |
| 92637_at | 24 | | Pfkl | phosphofructokinase, liver, B-type | IPR000023 // | PFK // |
| | | | | | Phosphofructokinase | Phosphofructokinase; |
| | | | | | | 8.2e−274 |
| 93143_at | 24 | | 1190005I06Rik | RIKEN cDNA 1190005I06 gene | — | — |
| 93304_at | 24 | | Slc3a1 | solute carrier family 3, member 1 | IPR006047 // Alpha | alpha-amylase // |
| | | | | | amylase, catalytic | Alpha amylase, |
| | | | | | domain | catalytic domain; 2.1e−64 |
| 96048_at | 24 | 6680277 | Hrsp12 | heat-responsive protein 12 | IPR006056 // YjgF- | ribonuc_L-PSP // |
| | | | | | like protein /// | Endoribonuclease L- |
| | | | | | IPR006175 // | PSP; 6.6e−65 |
| | | | | | Endoribonuclease L- | |
| | | | | | PSP | |
| 96956_at | 24 | | 0610038D11Rik | RIKEN cDNA 0610038D11 gene | IPR005651 // Protein | DUF343 // Protein of |
| | | | | | of unknown function | unknown function |
| | | | | | DUF343 /// | (DUF343); 5.7e−63 /// |
| | | | | | IPR000866 // Alkyl | AhpC-TSA // |
| | | | | | hydroperoxide | AhpC/TSA |
| | | | | | reductase/Thiol | family; 3.5e−08 |
| | | | | | specific antioxidant/ | |
| | | | | | Mal allergen | |
| 97316_at | 24 | 31541815 | 1300002P22Rik | RIKEN cDNA 1300002P22 gene | IPR006180 // 3- | 3HCDH // 3- |
| | | | | | hydroxyacyl-CoA | hydroxyacyl-CoA |
| | | | | | dehydrogenase /// | dehydrogenase, C- |
| | | | | | IPR006109 // NAD- | terminal; 2.2e−42 |
| | | | | | dependent glycerol-3- | |
| | | | | | phosphate | |
| | | | | | dehydrogenase | |
| | | | | | domain /// IPR001101 | |
| | | | | | // Plectin repeat /// | |
| | | | | | IPR001753 // Enoyl- | |
| | | | | | CoA | |
| | | | | | hydratase/isomerase | |
| | | | | | /// IPR006108 // 3- | |
| | | | | | hydroxyacyl-CoA | |
| | | | | | dehydrogenase, C- | |
| | | | | | terminal domain /// | |
| | | | | | IPR006176 // 3- | |
| | | | | | hydroxyacyl-CoA | |
| | | | | | dehydrogenase, NAD | |
| | | | | | binding domain /// | |
| | | | | | IPR001993 // | |
| | | | | | Mitochondrial | |
| | | | | | substrate carrier | |
| 98353_at | 24 | | Cyp4a10 | cytochrome P450, 4a10 | IPR001230 // Prenyl | — |
| | | | | | group binding site | |
| | | | | | (CAAX box) /// | |
| | | | | | IPR002402 // E-class | |
| | | | | | P450, group II /// | |
| | | | | | IPR001128 // | |
| | | | | | Cytochrome P450 /// | |
| | | | | | IPR002401 // E-class | |
| | | | | | P450, group I | |
| 99581_at | 24 | | Hint | histidine triad nucleotide binding | IPR001310 // | — |
| | | | | protein | Histidine triad (HIT) | |
| | | | | | protein | |
| 99894_at | 24 | | Ptgfrn | prostaglandin F2 receptor negative | IPR003600 // | ig // Immunoglobulin |
| | | | | regulator | Immunoglobulin-like | domain; 3e−33 |
| | | | | | /// IPR003006 // | |
| | | | | | Immunoglobulin/major | |
| | | | | | histocompatibility | |
| | | | | | complex /// | |
| | | | | | IPR003596 // | |
| | | | | | Immunoglobulin V- | |
| | | | | | type | |
| AFFX- | 24 | 6679937 | | | | |
| GapdhMur/ | | | | | | |
| M32599_M_st | | | | | | |
| 100828_at | 23 | | Myla | myosin light chain, alkali, cardiac | IPR002048 // | — |
| | | | | atria | Calcium-binding EF- | |
| | | | | | hand | |
| 100967_at | 23 | 6755548 | Slc27a2 | solute carrier family 27 (fatty acid | IPR000873 // AMP- | AMP-binding // AMP- |
| | | | | transporter), member 2 | dependent | binding enzyme; 2.3e−54 |
| | | | | | synthetase and ligase | |
| 101006_at | 23 | | Tcp1-rs1 | t-complex protein 1, related | IPR002155 // | — |
| | | | | sequence 1 | Thiolase | |
| 101531_at | 23 | | Aldo2 | aldolase 2, B isoform | IPR000741 // | glycolytic_enzy // |
| | | | | | Fructose- | Fructose- |
| | | | | | bisphosphate | bisphosphate aldolase |
| | | | | | aldolase, class-I | class-; 3.7e−243 |
| 101758_at | 23 | | Cktsf1b1 | cysteine knot superfamily 1, BMP | IPR000359 // | DAN // DAN |
| | | | | antagonist 1 | Cystine-knot domain | domain; 6.7e−79 |
| | | | | | /// IPR004133 // DAN | |
| | | | | | domain | |
| 102035_at | 23 | | Tpmt | thiopurine methyltransferase | — | — |
| 102636_at | 23 | | Klc2 | kinesin light chain 2 | IPR001440 // TPR | TPR // TPR |
| | | | | | repeat /// IPR002151 | Domain; 5.2e−20 |
| | | | | | // Kinesin light chain | |
| 102944_at | 23 | | — | Mus musculus 9 days embryo | — | — |
| | | | | whole body cDNA, RIKEN full- | | |
| | | | | length enriched library, | | |
| | | | | clone: D030073N12 | | |
| | | | | product: unknown EST, full insert | | |
| | | | | sequence. | | |
| 103333_at | 23 | | G6pc | glucose-6-phosphatase, catalytic | IPR000326 // PA- | PAP2 // PAP2 |
| | | | | | phosphatase related | superfamily; 8.4e−31 |
| | | | | | phosphoesterase | |
| 103618_at | 23 | | Ckmt2 | creatine kinase, mitochondrial 2 | — | — |
| 103703_f_at | 23 | | C730048C13Rik | RIKEN cDNA C730048C13 gene | — | — |
| 104255_at | 23 | | — | ESTs, Weakly similar to | — | — |
| | | | | DIA3_MOUSE Diaphanous | | |
| | | | | protein | | |
| | | | | homolog 3 (Diaphanous-related | | |
| | | | | formin 3) (DRF3) (mDIA2) | | |
| | | | | (p134mDIA2) [M. musculus] | | |
| 92826_at | 23 | | Gdap3 | ganglioside-induced | — | — |
| | | | | differentiation-associated- | | |
| | | | | protein 3 | | |
| 92835_at | 23 | | Cml1 | camello-like 1 | IPR000182 // GCN5- | Acetyltransf // |
| | | | | | related N- | Acetyltransferase |
| | | | | | acetyltransferase | (GNAT) family; 6.1e−16 |
| 93820_at | 23 | 31981830 | Cox7a2 | cytochrome c oxidase, | IPR003177 // | COX7a // Cytochrome |
| | | | | subunit VIIa 2 | Cytochrome c | c oxidase subunit |
| | | | | | oxidase, subunit VIIa | VIIa; 3.6e−52 |
| 94549_at | 23 | | 1200003O06Rik | RIKEN cDNA 1200003O06 gene | IPR005828 // General | sugar_tr // Sugar (and |
| | | | | | substrate transporter | other) |
| | | | | | | transporter; 0.0036 |
| 95588_at | 23 | 6678766 | Amacr | alpha-methylacyl-CoA racemase | IPR003673 // L- | CAIB-BAIF // |
| | | | | | carnitine | CAIB/BAIF |
| | | | | | dehydratase/bile | family; 6.6e−99 |
| | | | | | acid-inducible protein F | |
| 96072_at | 23 | | Ldh1 | lactate dehydrogenase 1, A chain | IPR001236 // | ldh // lactate/malate |
| | | | | | Lactate/malate | dehydrogenase, NAD |
| | | | | | dehydrogenase /// | binding do; 6.4e−82 /// |
| | | | | | IPR001557 // L- | ldh_C // lactate/malate |
| | | | | | lactate | dehydrogenase, |
| | | | | | dehydrogenase | alpha/beta C-t; 2.4e−87 |
| 96090_g_at | 23 | | 4931406C07Rik | RIKEN cDNA 4931406C07 gene | — | — |
| 96629_at | 23 | 14861848 | D7Rp2e | DNA segment, Chr 7, Roswell | IPR000086 // NUDIX | NUDIX // NUDIX |
| | | | | Park 2 complex, expressed | hydrolase | domain; 1.9e−14 |
| 97204_s_at | 23 | | 1110003P16Rik | RIKEN cDNA 1110003P16 gene | IPR001623 // Heat | DnaJ // DnaJ |
| | | | | | shock protein DnaJ, | domain; 4.8e−05 |
| | | | | | N-terminal | |
| 98457_at | 23 | | Slc4a4 | solute carrier family 4 (anion | IPR003020 // HCO3− | HCO3_cotransp // |
| | | | | exchanger), member 4 | transporter /// | HCO3− transporter |
| | | | | | IPR003024 // | family; 0 |
| | | | | | Na+/HCO3− co- | |
| | | | | | transporter /// | |
| | | | | | IPR001717 // Anion | |
| | | | | | exchange protein | |
| 98904_at | 23 | | 1110066C01Rik | RIKEN cDNA 1110066C01 gene | IPR001706 // | — |
| | | | | | Ribosomal protein | |
| | | | | | L35 | |
| 100916_at | 22 | | Slc22a1 | solute carrier family 22 (organic | IPR005829 // Sugar | sugar_tr // Sugar (and |
| | | | | cation transporter), member 1 | transporter | other) |
| | | | | | superfamily /// | transporter; 3.9e−10 |
| | | | | | IPR005828 // General | |
| | | | | | substrate transporter | |
| | | | | | /// IPR004749 // | |
| | | | | | Organic cation | |
| | | | | | transport protein | |
| 101897_g_at | 22 | | Cd1d2 | CD1d2 antigen | IPR003006 // | ig // Immunoglobulin |
| | | | | | Immunoglobulin/major | domain; 1.2e−05 |
| | | | | | histocompatibility | |
| | | | | | complex /// | |
| | | | | | IPR003597 // | |
| | | | | | Immunoglobulin C- | |
| | | | | | type | |
| 101964_at | 22 | | Tkt | transketolase | IPR005476 // | transketolase_C // |
| | | | | | Transketolase, C | Transketolase, C- |
| | | | | | terminal /// | terminal domain; 2.4e−34 |
| | | | | | IPR005475 // | /// transket_pyr // |
| | | | | | Transketolase, | Transketolase, |
| | | | | | central region /// | pyridine binding |
| | | | | | IPR005474 // | domai; 3.4e−55 /// |
| | | | | | Transketolase, N | transketolase // |
| | | | | | terminal | Transketolase, |
| | | | | | | thiamine diphosphate |
| | | | | | | b; 3.2e−154 |
| 102861_at | 22 | | Slc22a1l | solute carrier family 22 (organic | IPR001958 // | — |
| | | | | cation transporter), member 1-like | Tetracycline | |
| | | | | | resistance protein /// | |
| | | | | | IPR001226 // | |
| | | | | | Flavodoxin | |
| 102947_at | 22 | | Slc22a2 | solute carrier family 22 (organic | IPR005829 // Sugar | sugar_tr // Sugar (and |
| | | | | cation transporter), member 2 | transporter | other) |
| | | | | | superfamily /// | transporter; 7.3e−13 |
| | | | | | IPR005828 // General | |
| | | | | | substrate transporter | |
| | | | | | /// IPR004749 // | |
| | | | | | Organic cation | |
| | | | | | transport protein | |
| 103389_at | 22 | 31980703 | Aass | aminoadipate-semialdehyde | IPR005097 // | AlaDh_PNT // Alanine |
| | | | | synthase | Saccharopine | dehydrogenase/pyridine |
| | | | | | dehydrogenase /// | nucleotid; 1.9e−215 |
| | | | | | IPR004002 // Alanine | /// Saccharop_dh // |
| | | | | | dehydrogenase and | Saccharopine |
| | | | | | pyridine nucleotide | dehydrogenase; 0 |
| | | | | | transhydrogenase /// | |
| | | | | | IPR002016 // Haem | |
| | | | | | peroxidase | |
| 103580_at | 22 | | LOC215751 | similar to hypothetical protein | IPR001950 // | — |
| | | | | BC014320 | Translation initiation | |
| | | | | | factor SUI1 | |
| 104583_at | 22 | | 2400007G07Rik | RIKEN cDNA 2400007G07 gene | IPR001452 // SH3 | zf-DHHC // DHHC zinc |
| | | | | | domain /// IPR001594 | finger domain; 2.5e−27 |
| | | | | | // Zn-finger, DHHC | |
| | | | | | type | |
| 104584_f_at | 22 | | — | Mus musculus 8 days embryo | — | — |
| | | | | whole body cDNA, RIKEN full- | | |
| | | | | length enriched library, | | |
| | | | | clone: 5730439B18 | | |
| | | | | product: hypothetical protein, full | | |
| | | | | insert sequence. | | |
| 93045_at | 22 | | Abcd3 | ATP-binding cassette, sub-family | IPR003439 // ABC | ABC_tran // ABC |
| | | | | D (ALD), member 3 | transporter /// | transporter; 6.1e−27 |
| | | | | | IPR003593 // AAA | |
| | | | | | ATPase /// | |
| | | | | | IPR005283 // | |
| | | | | | Peroxysomal long | |
| | | | | | chain fatty acyl | |
| | | | | | transporter | |
| 93048_at | 22 | 8393156 | Clpp | caseinolytic protease, ATP- | IPR001907 // Clp | CLP_protease // Clp |
| | | | | dependent, proteolytic subunit | protease | protease; 2.3e−98 |
| | | | | homolog (E. coli) | | |
| 93084_at | 22 | | Slc25a4 | solute carrier family 25 | IPR002030 // | — |
| | | | | (mitochondrial carrier; adenine | Mitochondrial brown | |
| | | | | nucleotide translocator), member 4 | fat uncoupling protein | |
| | | | | | /// IPR002113 // | |
| | | | | | Adenine nucleotide | |
| | | | | | translocator 1 /// | |
| | | | | | IPR001993 // | |
| | | | | | Mitochondrial | |
| | | | | | substrate carrier /// | |
| | | | | | IPR002067 // | |
| | | | | | Mitochondrial carrier | |
| | | | | | protein | |
| 93431_at | 22 | | Dm15 | dystrophia myotonica kinase, B15 | — | pkinase // Protein |
| | | | | | | kinase domain; 4.1e−57 |
| 93570_at | 22 | | Slc12a3 | solute carrier family 12, member 3 | IPR002948 // | — |
| | | | | | Thiazide-sensitive | |
| | | | | | Na/Cl co-transporter | |
| | | | | | /// IPR004842 // K—Cl | |
| | | | | | cotransporter | |
| | | | | | superfamily /// | |
| | | | | | IPR002293 // Amino | |
| | | | | | acid/polyamine | |
| | | | | | transporter, family I | |
| 93736_at | 22 | | Tcn2 | transcobalamin 2 | IPR002157 // | Cobalamin_bind // |
| | | | | | Eukaryotic | Eukaryotic cobalamin- |
| | | | | | cobalamin-binding | binding protein; 2.1e−289 |
| | | | | | protein | |
| 93775_at | 22 | | D12Ertd647e | DNA segment, Chr 12, ERATO | — | — |
| | | | | Doi 647, expressed | | |
| 93826_at | 22 | | 2310028N02Rik | RIKEN cDNA 2310028N02 gene | IPR002554 // Protein | B56 // Protein |
| | | | | | phosphatase 2A, | phosphatase 2A |
| | | | | | regulatory B subunit | regulatory B |
| | | | | | (B56 family) | subunit; 8.7e−15 |
| 93832_at | 22 | | 5730443G10 | hypothetical protein 5730443G10 | — | R3H // R3H |
| | | | | | | domain; 1.5e−15 |
| 93833_s_at | 22 | | Hist1h2bc | histone 1, H2bc | IPR000558 // Histone | — |
| | | | | | H2B /// IPR004822 // | |
| | | | | | Histone-fold/TFIID- | |
| | | | | | TAF/NF-Y domain | |
| 93851_at | 22 | | Rabggta | Rab geranylgeranyl transferase, a | IPR002088 // Protein | PPTA // Protein |
| | | | | subunit | prenyltransferase, | prenyltransferase |
| | | | | | alpha subunit /// | alpha subunit |
| | | | | | IPR001611 // | repe; 6.3e−56 /// LRR |
| | | | | | Leucine-rich repeat | // Leucine Rich |
| | | | | | | Repeat; 2.5e−07 |
| 94419_at | 22 | | Slc19a1 | solute carrier family 19 | IPR002666 // | Folate_carrier // |
| | | | | (sodium/hydrogen exchanger), | Reduced folate | Reduced folate |
| | | | | member 1 | carrier | carrier; 1.8e−290 |
| 95119_at | 22 | | 1110038D17Rik | RIKEN cDNA 111003D17 gene | — | — |
| 95478_at | 22 | | Deb1 | differentially expressed | — | — |
| | | | | in B16F101 | | |
| 95620_at | 22 | | 2310016E22Rik | RIKEN cDNA 2310016E22 gene | IPR002198 // Short- | adh_short // short |
| | | | | | chain | chain |
| | | | | | dehydrogenase/reductase | dehydrogenase; 3.5e−45 |
| | | | | | SDR /// | |
| | | | | | IPR002347 // | |
| | | | | | Glucose/ribitol | |
| | | | | | dehydrogenase | |
| 95725_at | 22 | | 0610006H10Rik | RIKEN cDNA 0610006H10 gene | — | — |
| 96231_at | 22 | 21624609 | 2010012D11Rik | RIKEN cDNA 2010012D11 gene | IPR000073 // | abhydrolase // |
| | | | | | Alpha/beta hydrolase | alpha/beta hydrolase |
| | | | | | fold /// IPR003089 // | fold; 1.3e−19 |
| | | | | | Alpha/beta hydrolase | |
| | | | | | /// IPR000734 // | |
| | | | | | Lipase /// IPR000379 | |
| | | | | | // | |
| | | | | | Esterase/lipase/thioesterase, | |
| | | | | | active site | |
| 97525_at | 22 | 6680139 | Gyk | glycerol kinase | IPR005999 // | FGGY_C // FGGY |
| | | | | | Glycerol kinase /// | family of carbohydrate |
| | | | | | IPR000577 // | kinases, C-termi; 3.5e−110 |
| | | | | | Carbohydrate kinase, | /// FGGY // FGGY |
| | | | | | FGGY | family of carbohydrate |
| | | | | | | kinases, N-termi; 6.5e−135 |
| 97533_at | 22 | | Fcgrt | Fc receptor, IgG, alpha chain | IPR001220 // | MHC_I // Class I |
| | | | | transporter | Legume lectin, beta | Histocompatibility |
| | | | | | domain /// IPR001039 | antigen, |
| | | | | | // Major | domains; 2.7e−72 |
| | | | | | histocompatibility | |
| | | | | | complex protein, | |
| | | | | | class I /// IPR003006 | |
| | | | | | // | |
| | | | | | Immunoglobulin/major | |
| | | | | | histocompatibility | |
| | | | | | complex /// | |
| | | | | | IPR003597 // | |
| | | | | | Immunoglobulin C- | |
| | | | | | type | |
| 98124_at | 22 | | 0610011F06Rik | RIKEN cDNA 0610011F06 gene | — | — |
| 98482_at | 22 | | Pthr1 | parathyroid hormone receptor 1 | IPR002170 // | 7tm_2 // 7 |
| | | | | | Parathyroid hormone | transmembrane |
| | | | | | receptor /// | receptor (Secretin |
| | | | | | IPR001879 // | family); 2.8e−129 /// |
| | | | | | Hormone receptor, | HRM // Hormone |
| | | | | | extracellular /// | receptor domain; 9.1e−26 |
| | | | | | IPR000832 // G- | |
| | | | | | protein coupled | |
| | | | | | receptors family 2 | |
| | | | | | (secretin-like) | |
| 99112_at | 22 | 7305501 | Slc25a10 | solute carrier family 25 | IPR002030 // | mito_carr // |
| | | | | (mitochondrial carrier; | Mitochondrial brown | Mitochondrial carrier |
| | | | | dicarboxylate transporter), | fat uncoupling protein | protein; 4.3e−70 |
| | | | | member 10 | /// IPR001993 // | |
| | | | | | Mitochondrial | |
| | | | | | substrate carrier | |
| 99115_at | 22 | 21539599 | 2610041P16Rik | RIKEN cDNA 2610041P16 gene | IPR003422 // | UCR_hinge // |
| | | | | | Ubiquinol-cytochrome | Ubiquinol-cytochrome |
| | | | | | C reductase hinge | C reductase hinge |
| | | | | | protein | prot; 2.6e−42 |
| 99959_at | 22 | 6753022 | Ak4 | adenylate kinase 4 | IPR000850 // | adenylatekinase // |
| | | | | | Adenylate kinase | Adenylate |
| | | | | | | kinase; 2.3e−102 |
| 99974_at | 22 | | Kcnj15 | potassium inwardly-rectifying | IPR001622 // K+ | IRK // Inward rectifier |
| | | | | channel, subfamily J, member 15 | channel, pore region | potassium |
| | | | | | /// IPR001838 // K+ | channel; 2.2e−221 |
| | | | | | channel, inward | |
| | | | | | rectifier /// | |
| | | | | | IPR003270 // Kir1.3 | |
| | | | | | inward rectifier K+ | |
| | | | | | channel | |
| AFFX- | 22 | 6679237 | | | | |
| PyruCarbMur/ | | | | | | |
| L09192_3_at | | | | | | |
| 100567_at | 21 | | Fabp4 | fatty acid binding protein 4, | IPR000463 // | lipocalin // Lipocalin/ |
| | | | | adipocyte | Cytosolic fatty-acid | cytosolic fatty-acid |
| | | | | | binding protein /// | binding pr; 3e−39 |
| | | | | | IPR000566 // | |
| | | | | | Lipocalin-related | |
| | | | | | protein and | |
| | | | | | Bos/Can/Equ | |
| | | | | | allergen | |
| 100986_at | 21 | | Fhl2 | four and a half LIM domains 2 | IPR001781 // Zn- | LIM // LIM |
| | | | | | binding protein, LIM | domain; 1.2e−34 |
| 101029_f_at | 21 | | Actc1 | actin, alpha, cardiac | IPR004000 // | actin // Actin; 1.2e−276 |
| | | | | | Actin/actin-like /// | |
| | | | | | IPR004001 // Actin | |
| 101299_at | 21 | | — | — | — | — |
| 101394_at | 21 | | Sgcg | sarcoglycan, gamma (35 kD | — | — |
| | | | | dystrophin-associated | | |
| | | | | glycoprotein) | | |
| 101872_at | 21 | | Gsta2 | glutathione S-transferase, alpha 2 | IPR004045 // | GST_N // Glutathione |
| | | | | (Yc2) | Glutathione S- | S-transferase, N- |
| | | | | | transferase, N- | terminal domain; 2.4e−25 |
| | | | | | terminal /// | /// GST_C // |
| | | | | | IPR003080 // | Glutathione S- |
| | | | | | Glutathione S- | transferase, C-terminal |
| | | | | | transferase, alpha | domain; 3.3e−30 |
| | | | | | class /// IPR004046 | |
| | | | | | // Glutathione S- | |
| | | | | | transferase, C- | |
| | | | | | terminal | |
| 102114_f_at | 21 | | Angptl4 | angiopoietin-like 4 | IPR002181 // | fibrinogen_C // |
| | | | | | Fibrinogen, | Fibrinogen beta and |
| | | | | | beta/gamma chain, | gamma chains, C- |
| | | | | | C-terminal globular | term; 4.8e−58 |
| 102886_at | 21 | | Gpc4 | glypican 4 | IPR001863 // | Glypican // Glypican; 0 |
| | | | | | Glypican | |
| 103602_at | 21 | | Dao1 | D-amino acid oxidase | IPR006181 // D- | DAO // FAD |
| | | | | | amino acid oxidase /// | dependent |
| | | | | | IPR006076 // FAD | oxidoreductase; 1.7e−133 |
| | | | | | dependent | |
| | | | | | oxidoreductase /// | |
| | | | | | IPR001412 // | |
| | | | | | Aminoacyl-tRNA | |
| | | | | | synthetase, class I | |
| 103879_at | 21 | | LOC235169 | hypothetical protein LOC235169 | IPR006076 // FAD | DAO // FAD |
| | | | | | dependent | dependent |
| | | | | | oxidoreductase | oxidoreductase; 0.0011 |
| 103955_at | 21 | | Cryl1 | crystallin, lamda 1 | IPR006180 // 3- | 3HCDH // 3- |
| | | | | | hydroxyacyl-CoA | hydroxyacyl-CoA |
| | | | | | dehydrogenase /// | dehydrogenase, C- |
| | | | | | IPR006109 // NAD- | terminal; 3.5e−22 /// |
| | | | | | dependent glycerol-3- | 3HCDH_N // 3- |
| | | | | | phosphate | hydroxyacyl-CoA |
| | | | | | dehydrogenase | dehydrogenase, NAD |
| | | | | | domain /// IPR000205 | binding; 1.2e−86 |
| | | | | | // NAD binding site /// | |
| | | | | | IPR006108 // 3- | |
| | | | | | hydroxyacyl-CoA | |
| | | | | | dehydrogenase, C- | |
| | | | | | terminal domain /// | |
| | | | | | IPR006176 // 3- | |
| | | | | | hydroxyacyl-CoA | |
| | | | | | dehydrogenase, NAD | |
| | | | | | binding domain | |
| 104258_at | 21 | | Acyp2 | acylphosphatase 2, muscle type | IPR002048 // | Acylphosphatase // |
| | | | | | Calcium-binding EF- | Acylphosphatase; 2.9e−59 |
| | | | | | hand /// IPR001792 // | |
| | | | | | Acylphosphatase | |
| 104387_at | 21 | | Slc23a2 | solute carrier family 23 | IPR006043 // | xan_ur_permease // |
| | | | | (nucleobase transporters), | Xanthine/uracil/vitamin | Permease family; 9.2e−94 |
| | | | | member 2 | C permease family | |
| 104706_at | 21 | | Pex7 | peroxisome biogenesis factor 7 | IPR001680 // G- | WD40 // WD domain, |
| | | | | | protein beta WD-40 | G-beta repeat; 3.9e−49 |
| | | | | | repeat | |
| 92814_at | 21 | | Cyp2j5 | cytochrome P450, 2j5 | IPR001128 // | p450 // Cytochrome |
| | | | | | Cytochrome P450 /// | P450; 1.5e−165 |
| | | | | | IPR002401 // E-class | |
| | | | | | P450, group I | |
| 92869_at | 21 | 6680291 | Hsd3b4 | hydroxysteroid dehydrogenase-4, | IPR002225 // 3-beta | 3Beta_HSD // 3-beta |
| | | | | delta<5>-3-beta | hydroxysteroid | hydroxysteroid |
| | | | | | dehydrogenase/isomerase | dehydrogenase/isomera; |
| | | | | | | 1.8e−203 |
| 93221_at | 21 | | 4921540P06Rik | RIKEN cDNA 4921540P06 gene | IPR001356 // | — |
| | | | | | Homeobox /// | |
| | | | | | IPR001827 // | |
| | | | | | Homeobox protein, | |
| | | | | | antennapedia type | |
| 93542_at | 21 | | Pter | phosphotriesterase related | IPR001559 // | PTE // |
| | | | | | Aryldialkylphosphatase | Phosphotriesterase |
| | | | | | | family; 8.9e−239 |
| 93629_s_at | 21 | | Folh1 | folate hydrolase | IPR003137 // | PA // PA domain; 8.6e−21 |
| | | | | | Protease-associated | /// TFR_dimer // |
| | | | | | PA | Transferring receptor- |
| | | | | | | like dimerisation |
| | | | | | | dom; 3.8e−65 |
| 93696_at | 21 | | Nr1i2 | nuclear receptor subfamily 1, | IPR001628 // Zn- | — |
| | | | | group I, member 2 | finger, C4-type | |
| | | | | | steroid receptor /// | |
| | | | | | IPR000324 // Vitamin | |
| | | | | | D receptor /// | |
| | | | | | IPR001723 // Steroid | |
| | | | | | hormone receptor /// | |
| | | | | | IPR000536 // Ligand- | |
| | | | | | binding domain of | |
| | | | | | nuclear hormone | |
| | | | | | receptor | |
| 93781_at | 21 | | Aldrl6 | aldehyde reductase (aldose | IPR001395 // | — |
| | | | | reductase)-like 6 | Aldo/keto reductase | |
| 94199_at | 21 | | Kap | kidney androgen regulated protein | — | — |
| 94241_at | 21 | | 1300003G02Rik | RIKEN cDNA 1300003G02 gene | IPR001977 // | CoaE // Dephospho- |
| | | | | | Dephospho-CoA | CoA kinase; 3.1e−87 |
| | | | | | kinase | /// CTP_transf_2 // |
| | | | | | | Cytidylyltransferase; |
| | | | | | | 2.3e−08 |
| 94435_at | 21 | | D10Ertd438e | DNA segment, Chr 10, ERATO | — | — |
| | | | | Doi 438, expressed | | |
| 95028_r_at | 21 | | — | — | — | — |
| 95074_at | 21 | | Pxf | peroxisomal farnesylated protein | IPR001230 // Prenyl | — |
| | | | | | group binding site | |
| | | | | | (CAAX box) | |
| 95539_at | 21 | | Gtpat12 | gene trap PAT 12 | — | — |
| 96069_at | 21 | 27659728 | Afar | aflatoxin B1 aldehyde reductase | IPR001395 // | aldo_ket_red // |
| | | | | | Aldo/keto reductase | Aldo/keto reductase |
| | | | | | | family; 3e−14 |
| 96078_g_at | 21 | | Slc17a1 | solute carrier family 17 vesicular | IPR005828 // General | — |
| | | | | glutamate transporter), member 1 | substrate transporter | |
| | | | | | /// IPR004745 // | |
| | | | | | Na(+)-dependent | |
| | | | | | inorganic phosphate | |
| | | | | | cotransporter | |
| 96888_at | 21 | | Akr1a4 | aldo-keto reductase family 1, | IPR001395 // | aldo_ket_red // |
| | | | | member A4 (aldehyde reductase) | Aldo/keto reductase | Aldo/keto reductase |
| | | | | | | family; 1.1e−147 |
| 97001_r_at | 21 | | Olfr37c | olfactory receptor 37c | — | 7tm_1 // 7 |
| | | | | | | transmembrane |
| | | | | | | receptor (rhodopsin |
| | | | | | | family); 2.2e−38 |
| 97089_at | 21 | | Folh1 | folate hydrolase | IPR003137 // | PA // PA domain; 8.6e−21 |
| | | | | | Protease-associated | /// TFR_dimer // |
| | | | | | PA | Transferring receptor- |
| | | | | | | like dimerisation |
| | | | | | | dom; 3.8e−65 |
| 97287_at | 21 | | 4933412D19Rik | RIKEN cDNA 4933412D19 gene | — | — |
| 97342_at | 21 | 13384894 | Mrps14 | mitochondrial ribosomal protein | IPR001209 // | Ribosomal_S14 // |
| | | | | S14 | Ribosomal protein | Ribosomal protein |
| | | | | | S14 | S14p/S29e; 1.6e−18 |
| 97514_at | 21 | | 1810063B05Rik | RIKEN cDNA 1810063B05 gene | — | — |
| 98131_at | 21 | | Cryz | crystallin, zeta | IPR002085 // Zinc- | — |
| | | | | | containing alcohol | |
| | | | | | dehydrogenase | |
| | | | | | superfamily /// | |
| | | | | | IPR002364 // | |
| | | | | | Quinone | |
| | | | | | oxidoreductase/zeta- | |
| | | | | | crystallin | |
| 99107_at | 21 | | Ghr | growth hormone receptor | IPR002996 // | — |
| | | | | | Cytokine receptor, | |
| | | | | | common beta/gamma | |
| | | | | | chain /// IPR003528 // | |
| | | | | | Long hematopoietin | |
| | | | | | receptor, single chain | |
| 99402_at | 21 | | Art2b | ADP-ribosyltransferase 2b | IPR000768 // | ART // NAD:arginine |
| | | | | | NAD:arginine ADP- | ADP- |
| | | | | | ribosyltransferase, | ribosyltransferase; |
| | | | | | ART | 1.3e−147 |
| 100085_at | 20 | | Ggtp | gamma-glutamyl transpeptidase | IPR000101 // | G_glu_transpept // |
| | | | | | Gamma- | Gamma- |
| | | | | | glutamyltranspeptidase | glutamyltranspeptidase; |
| | | | | | | 3.1e−273 |
| 100909_at | 20 | | Prss8 | protease, serine, 8 (prostasin) | IPR001314 // | trypsin // Trypsin; 4.6e−90 |
| | | | | | Chymotrypsin serine | |
| | | | | | protease, family S1 /// | |
| | | | | | IPR001254 // Serine | |
| | | | | | protease, trypsin | |
| | | | | | family | |
| 100913_at | 20 | | Thea | thioesterase, adipose associated | IPR002590 // Acyl- | START // START |
| | | | | | CoA thioester | domain; 6.4e−25 /// |
| | | | | | hydrolase, cytosolic | Acyl-CoA_hydro // |
| | | | | | long chain /// | Cytosolic long-chain |
| | | | | | IPR002913 // Lipid- | acyl-CoA |
| | | | | | binding START | thioeste; 1.4e−34 |
| 100956_at | 20 | | Kl | klotho | IPR001360 // | Glyco_hydro_1 // |
| | | | | | Glycoside hydrolase, | Glycosyl hydrolase |
| | | | | | family 1 | family 1; 1e−203 |
| 101539_f_at | 20 | | Ces3 | carboxylesterase 3 | IPR002018 // | COesterase // |
| | | | | | Carboxylesterase, | Carboxylesterase; 2.5e−206 |
| | | | | | type B /// IPR000379 | |
| | | | | | // | |
| | | | | | Esterase/lipase/thioesterase, | |
| | | | | | active site | |
| 101659_at | 20 | | Hsd3b2 | hydroxysteroid dehydrogenase-2, | IPR002225 // 3-beta | 3Beta_HSD // 3-beta |
| | | | | delta<5>-3-beta | hydroxysteroid | hydroxysteroid |
| | | | | | dehydrogenase/isomerase | dehydrogenase/isomera; |
| | | | | | | 2.3e−209 |
| 101907_s_at | 20 | | Ceacam2 | CEA-related cell adhesion | IPR003599 // | ig // Immunoglobulin |
| | | | | molecule 2 | Immunoglobulin | domain; 6.6e−05 |
| | | | | | subtype /// | |
| | | | | | IPR003598 // | |
| | | | | | Immunoglobulin C-2 | |
| | | | | | type /// IPR003006 // | |
| | | | | | Immunoglobulin/major | |
| | | | | | histocompatibility | |
| | | | | | complex | |
| 101972_at | 20 | | Kdap | kidney-derived aspartic protease- | IPR001969 // | asp // Eukaryotic |
| | | | | like protein | Eukaryotic/viral | aspartyl |
| | | | | | aspartic protease, | protease; 7.6e−147 |
| | | | | | active site /// | |
| | | | | | IPR001461 // | |
| | | | | | Aspartic protease A1, | |
| | | | | | pepsin | |
| 102192_r_at | 20 | 31982720 | Sah | SA rat hypertension-associated | IPR000873 // AMP- | AMP-binding // AMP- |
| | | | | homolog | dependent | binding enzyme; 1.2e−102 |
| | | | | | synthetase and ligase | |
| 102429_at | 20 | | Slc22al2 | solute carrier family 22 (organic | IPR005828 // General | sugar_tr // Sugar (and |
| | | | | cation transporter)-like 2 | substrate transporter | other) |
| | | | | | | transporter; 8.2e−08 |
| 103353_f_at | 20 | | Cyp4b1 | cytochrome P450, subfamily IV B, | IPR001128 // | p450 // Cytochrome |
| | | | | polypeptide 1 | Cytochrome P450 /// | P450; 2.9e−144 |
| | | | | | IPR002401 // E-class | |
| | | | | | P450, group I | |
| 103377_at | 20 | | Lrp2 | low density lipoprotein receptor- | IPR000033 // Low- | — |
| | | | | related protein 2 | density lipoprotein | |
| | | | | | receptor, YWTD | |
| | | | | | repeat | |
| 103570_at | 20 | | Cors-pending | collagenous repeat-containing | IPR000087 // | Collagen // Collagen |
| | | | | sequence | Collagen triple helix | triple helix repeat (20 |
| | | | | | repeat /// IPR001073 | copies); 1e−10 /// C1q |
| | | | | | // Complement C1q | // C1q domain; 7.7e−18 |
| | | | | | protein | |
| 103973_at | 20 | | Kcnj1 | potassium inwardly-rectifying | IPR001622 // K+ | IRK // Inward rectifier |
| | | | | channel, subfamily J, member 1 | channel, pore region | potassium |
| | | | | | /// IPR001838 // K+ | channel; 1.4e−231 |
| | | | | | channel, inward | |
| | | | | | rectifier /// | |
| | | | | | IPR003268 // Kir1.1 | |
| | | | | | inward rectifier K+ | |
| | | | | | channel | |
| 103984_at | 20 | | — | Mus musculus 0 day neonate | — | — |
| | | | | kidney cDNA, RIKEN full-length | | |
| | | | | enriched library, | | |
| | | | | clone: D630026G14 | | |
| | | | | product: hypothetical protein, full | | |
| | | | | insert sequence. | | |
| 104164_at | 20 | | 1300019N10Rik | RIKEN cDNA 1300019N10 gene | IPR000126 // Serine | — |
| | | | | | proteases, V8 family | |
| | | | | | /// IPR001254 // | |
| | | | | | Serine protease, | |
| | | | | | trypsin family | |
| 104381_at | 20 | | Nr1h3 | nuclear receptor subfamily 1, | IPR001628 // Zn- | zf-C4 // Zinc finger, C4 |
| | | | | group H, member 3 | finger, C4-type | type (two |
| | | | | | steroid receptor /// | domains); 5.5e−38 /// |
| | | | | | IPR003069 // | hormone_rec // |
| | | | | | Ecdysteroid receptor | Ligand-binding domain |
| | | | | | /// IPR001723 // | of nuclear |
| | | | | | Steroid hormone | hormone; 4.8e−52 |
| | | | | | receptor /// | |
| | | | | | IPR000536 // Ligand- | |
| | | | | | binding domain of | |
| | | | | | nuclear hormone | |
| | | | | | receptor /// | |
| | | | | | IPR000923 // Blue | |
| | | | | | (type 1) copper | |
| | | | | | domain | |
| 104565_at | 20 | | Ap4s1 | adaptor-related protein complex | IPR000804 // Clathrin | Clat_adaptor_s // |
| | | | | AP-4, sigma 1 | adaptor complex, | Clathrin adaptor |
| | | | | | small chain | complex small |
| | | | | | | chain; 1.7e−49 |
| 92375_at | 20 | | 1810015P09Rik | RIKEN cDNA 1810015P09 gene | IPR004088 // KH | — |
| | | | | | domain, type 1 /// | |
| | | | | | IPR004087 // KH | |
| | | | | | domain | |
| 92561_at | 20 | | Entpd5 | ectonucleoside triphosphate | IPR000407 // | GDA1_CD39 // |
| | | | | diphosphohydrolase 5 | GDA1/CD39 family of | GDA1/CD39 |
| | | | | | nucleoside | (nucleoside |
| | | | | | phosphatase | phosphatase) |
| | | | | | | family; 7.3e−44 |
| 93515_at | 20 | | Cdh16 | cadherin 16 | IPR002126 // | cadherin // Cadherin |
| | | | | | Cadherin /// | domain; 2.3e−54 |
| | | | | | IPR001412 // | |
| | | | | | Aminoacyl-tRNA | |
| | | | | | synthetase, class I | |
| 94126_at | 20 | | Wnt2b | wingless related MMTV | IPR005817 // Wnt | wnt // wnt family; 4.8e−194 |
| | | | | integration | superfamily /// | |
| | | | | site 2b | IPR005816 // | |
| | | | | | Secreted growth | |
| | | | | | factor Wnt protein | |
| 94337_at | 20 | | Gas2 | growth arrest specific 2 | IPR003108 // Growth- | GAS2 // Growth- |
| | | | | | arrest-specific protein | Arrest-Specific Protein |
| | | | | | 2 /// IPR001715 // | 2 Domain; 3.5e−53 /// |
| | | | | | Calponin-like actin- | CH // Calponin |
| | | | | | binding | homology (CH) |
| | | | | | | domain; 9.4e−08 |
| 94338_g_at | 20 | | Gas2 | growth arrest specific 2 | IPR003108 // Growth- | GAS2 // Growth- |
| | | | | | arrest-specific protein | Arrest-Specific Protein |
| | | | | | 2 /// IPR001715 // | 2 Domain; 3.5e−53 /// |
| | | | | | Calponin-like actin- | CH // Calponin |
| | | | | | binding | homology (CH) |
| | | | | | | domain; 9.4e−08 |
| 94424_at | 20 | | Scd1 | stearoyl-Coenzyme A desaturase 1 | IPR001522 // Fatty | FA_desaturase // Fatty |
| | | | | | acid desaturase, type | acid desaturase; 5.2e−80 |
| | | | | | 1 // IPR005804 // | |
| | | | | | Fatty acid desaturase | |
| | | | | | family | |
| 94518_at | 20 | | 0610033H09Rik | RIKEN cDNA 0610033H09 gene | — | — |
| 94827_at | 20 | | Fxyd2 | FXYD domain-containing ion | — | ATP1G1_PLM_MAT8 |
| | | | | transport regulator 2 | | // ATP1G1/PLM/MAT8 |
| | | | | | | family; 2.9e−33 |
| 95594_at | 20 | | 6330416C07Rik | RIKEN cDNA 6330416C07 gene | — | — |
| 96605_at | 20 | | 0610011I04Rik | RIKEN cDNA 0610011I04 gene | — | — |
| 96684_at | 20 | | D5Wsu31e | DNA segment, Chr 5, Wayne State | — | — |
| | | | | University 31, expressed | | |
| 96790_f_at | 20 | | A530057M15Rik | RIKEN cDNA A530057M15 gene | — | — |
| 96935_at | 20 | | Map17-pending | membrane-associated protein 17 | — | — |
| 97288_at | 20 | | Pdzk1 | PDZ domain containing 1 | IPR001478 // | PDZ // PDZ domain |
| | | | | | PDZ/DHR/GLGF | (Also known as DHR |
| | | | | | domain | or GLGF); 8.8e−50 |
| 97886_at | 20 | | Spr | sepiapterin reductase | IPR002198 // Short- | adh_short // short |
| | | | | | chain | chain |
| | | | | | dehydrogenase/reductase | dehydrogenase; 1e−07 |
| | | | | | SDR /// | |
| | | | | | IPR002347 // | |
| | | | | | Glucose/ribitol | |
| | | | | | dehydrogenase | |
| 98123_at | 20 | 6754408 | Kat2 | kynurenine aminotransferase II | — | — |
| 98575_at | 20 | | Fasn | fatty acid synthase | IPR001031 // | — |
| | | | | | Thioesterase /// | |
| | | | | | IPR000051 // SAM | |
| | | | | | (and some other | |
| | | | | | nucleotide) binding | |
| | | | | | motif /// IPR002085 // | |
| | | | | | Zinc-containing | |
| | | | | | alcohol | |
| | | | | | dehydrogenase | |
| | | | | | superfamily /// | |
| | | | | | IPR000794 // Beta- | |
| | | | | | ketoacyl synthase /// | |
| | | | | | IPR006162 // | |
| | | | | | Phosphopantetheine | |
| | | | | | attachment site /// | |
| | | | | | IPR001227 // Acyl | |
| | | | | | transferase /// | |
| | | | | | IPR006163 // | |
| | | | | | Phosphopantetheine- | |
| | | | | | binding domain | |
| 99019_at | 20 | | Por | P450 (cytochrome) oxidoreductase | IPR001094 // | NAD_binding_1 // |
| | | | | | Flavodoxin-like | Oxidoreductase NAD- |
| | | | | | domain /// IPR003097 | binding domain; 7.8e−44 |
| | | | | | // FAD-binding /// | /// FAD_binding_1 |
| | | | | | IPR001433 // | // FAD binding |
| | | | | | Oxidoreductase | domain; 5e−121 /// |
| | | | | | FAD/NAD(P)-binding | flavodoxin // |
| | | | | | /// IPR001709 // | Flavodoxin; 1e−55 |
| | | | | | Flavoprotein pyridine | |
| | | | | | nucleotide | |
| | | | | | cytochrome | |
| | | | | | reductase /// | |
| | | | | | IPR001226 // | |
| | | | | | Flavodoxin | |
| 99070_at | 20 | | Chuk | conserved helix-loop-helix | IPR001245 // | pkinase // Protein |
| | | | | ubiquitous kinase | Tyrosine protein | kinase domain; 1.3e−48 |
| | | | | | kinase /// IPR002290 | |
| | | | | | // Serine/Threonine | |
| | | | | | protein kinase /// | |
| | | | | | IPR000719 // | |
| | | | | | Eukaryotic protein | |
| | | | | | kinase | |
| 99094_at | 20 | | Slc12a1 | solute carrier family 12, member 1 | IPR004841 // Domain | aa_permeases // |
| | | | | | found in permeases | Amino acid |
| | | | | | /// IPR002443 // | permease; 0.56 |
| | | | | | Na—K—Cl | |
| | | | | | co-transporter /// | |
| | | | | | IPR002445 // Na—K—Cl | |
| | | | | | co-transporter 2 /// | |
| | | | | | IPR004842 // K—Cl | |
| | | | | | cotransporter | |
| | | | | | superfamily /// | |
| | | | | | IPR002293 // Amino | |
| | | | | | acid/polyamine | |
| | | | | | transporter, family I | |
| 99521_at | 20 | 6753022 | Ak4 | adenylate kinase 4 | IPR000850 // | adenylatekinase // |
| | | | | | Adenylate kinase | Adenylate |
| | | | | | | kinase; 2.3e−102 |
| 99525_at | 20 | | Slc8a1 | solute carrier family 8 | IPR004836 // | Na_Ca_Ex // |
| | | | | (sodium/calcium exchanger), | Sodium/calcium | Sodium/calcium |
| | | | | member 1 | exchanger protein /// | exchanger |
| | | | | | IPR002987 // | protein; 4.8e−70 /// |
| | | | | | Sodium/calcium | Calx-beta // Calx-beta |
| | | | | | exchanger, isoform 1 | domain; 2.2e−84 |
| | | | | | /// IPR001623 // Heat | |
| | | | | | shock protein DnaJ, | |
| | | | | | N-terminal /// | |
| | | | | | IPR003644 // Na—Ca | |
| | | | | | exchanger/integrin- | |
| | | | | | beta4 /// IPR004837 | |
| | | | | | // Sodium/calcium | |
| | | | | | exchanger | |
| | | | | | membrane region | |
| 99966_at | 20 | | — | Mus musculus 2 days neonate | — | — |
| | | | | thymus thymic | | |
| | | | | cells cDNA, RIKEN | | |
| | | | | full-length enriched library, | | |
| | | | | clone: E430007C20 | | |
| | | | | product: weakly | | |
| | | | | similar to ACTIN INTERACTING | | |
| | | | | PROTEIN [Arabidopsis thaliana], | | |
| | | | | full insert sequence. | | |
| AFFX- | 20 | 6679937 | | | | |
| GapdhMur/ | | | | | | |
| M32599_5_at | | | | | | |
| AFFX- | 20 | 6679237 | | | | |
| PyruCarbMur/ | | | | | | |
| L09192_MB_at | | | | | | |
| 100040_at | 19 | | Mrpl17 | mitochondrial ribosomal protein | IPR000456 // | Ribosomal_L17 // |
| | | | | L17 | Ribosomal protein | Ribosomal protein |
| | | | | | L17 | L17; 5.3e−20 |
| 100491_at | 19 | | Slc16a2 | solute carrier family 16 | — | — |
| | | | | (monocarboxylic acid | | |
| | | | | transporters), member 2 | | |
| 100542_at | 19 | | Mep1a | meprin 1 alpha | IPR001506 // Astacin | Astacin // Astacin |
| | | | | | /// IPR000998 // MAM | (Peptidase family |
| | | | | | domain /// IPR003007 | M12A); 8.1e−93 /// |
| | | | | | // Meprin A, C- | MAM // MAM |
| | | | | | terminal TRAF /// | domain; 1.4e−62 /// |
| | | | | | IPR006025 // Neutral | EGF // EGF-like |
| | | | | | zinc | domain; 1.3e−10 /// |
| | | | | | metallopeptidases, | MATH // MATH |
| | | | | | zinc-binding region /// | domain; 6.4e−24 |
| | | | | | IPR000561 // EGF- | |
| | | | | | like domain /// | |
| | | | | | IPR006026 // Neutral | |
| | | | | | zinc metallopeptidase | |
| | | | | | /// IPR003006 // | |
| | | | | | Immunoglobulin/major | |
| | | | | | histocompatibility | |
| | | | | | complex /// | |
| | | | | | IPR002083 // | |
| | | | | | Meprin/TRAF-like | |
| | | | | | MATH | |
| 101086_f_at | 19 | | Cnbp | cellular nucleic acid | IPR001878 // Zn- | zf-CCHC // Zinc |
| | | | | binding protein | finger, CCHC type | knuckle; 1.4e−51 |
| 101396_at | 19 | | Tcf2 | transcription factor 2 | IPR001356 // | — |
| | | | | | Homeobox | |
| 101552_at | 19 | | Slc34a1 | solute carrier family 34 (sodium | IPR003841 // Na+/Pi− | Na_Pi_cotrans // |
| | | | | phosphate), member 1 | cotransporter | Na+/Pi− |
| | | | | | | cotransporter; 5.4e−209 |
| 102053_at | 19 | | Plscr2 | phospholipid scramblase 2 | IPR005552 // | Scramblase // |
| | | | | | Scramblase | Scramblase; 4.7e−130 |
| 103083_at | 19 | | Lipe | lipase, hormone sensitive | IPR002168 // | — |
| | | | | | Lipolytic enzyme /// | |
| | | | | | IPR000379 // | |
| | | | | | Esterase/lipase/thioe | |
| | | | | | sterase, active site | |
| 103972_at | 19 | | Kcnj1 | potassium inwardly-rectifying | IPR001622 // K+ | IRK // Inward rectifier |
| | | | | channel, subfamily J, member 1 | channel, pore region | potassium |
| | | | | | /// IPR001838 // K+ | channel; 1.4e−231 |
| | | | | | channel, inward | |
| | | | | | rectifier /// | |
| | | | | | IPR003268 // Kir1.1 | |
| | | | | | inward rectifier K+ | |
| | | | | | channel | |
| 104060_at | 19 | | 2700088M22Rik | RIKEN cDNA 2700088M22 gene | IPR000504 // RNA- | zf-CCHC // Zinc |
| | | | | | binding region RNP-1 | knuckle; 0.00063 /// |
| | | | | | (RNA recognition | rrm // RNA recognition |
| | | | | | motif) /// IPR001878 | motif. (a.k.a. RRM, |
| | | | | | // Zn-finger, CCHC | RBD, or; 8.6e−22 |
| | | | | | type | |
| 104076_at | 19 | | 1190017O12Rik | RIKEN cDNA 1190017O12 gene | — | — |
| 104138_at | 19 | | 2310074E22Rik | RIKEN cDNA 2310074E22 gene | — | — |
| 104603_at | 19 | | Gstt2 | glutathione S-transferase, theta 2 | IPR004045 // | GST_N // Glutathione |
| | | | | | Glutathione S- | S-transferase, N- |
| | | | | | transferase, N- | terminal domain; 3.7e−11 |
| | | | | | terminal /// | /// GST_C // |
| | | | | | IPR004046 // | Glutathione S- |
| | | | | | Glutathione S- | transferase, C-terminal |
| | | | | | transferase, C- | domain; 1.3e−24 |
| | | | | | terminal | |
| 92382_at | 19 | | Myo6 | myosin VI | IPR000048 // IQ | myosin_head // |
| | | | | | calmodulin-binding | Myosin head (motor |
| | | | | | region /// IPR001609 | domain); 6.4e−249 |
| | | | | | // Myosin head (motor | |
| | | | | | domain) | |
| 92605_at | 19 | | Umod | uromodulin | IPR001881 // EGF- | zona_pellucida // Zona |
| | | | | | like calcium-binding | pellucida-like |
| | | | | | /// IPR000152 // | domain; 3.4e−93 /// |
| | | | | | Aspartic acid and | EGF // EGF-like |
| | | | | | asparagine | domain; 2.5e−12 |
| | | | | | hydroxylation site /// | |
| | | | | | IPR001507 // | |
| | | | | | Endoglin/CD105 | |
| | | | | | antigen /// IPR000561 | |
| | | | | | // EGF-like domain /// | |
| | | | | | IPR000345 // | |
| | | | | | Cytochrome c heme- | |
| | | | | | binding site | |
| 93053_at | 19 | | Casq2 | calsequestrin 2 | IPR001393 // | Calsequestrin // |
| | | | | | Calsequestrin | Calsequestrin; 1.6e−267 |
| 93320_at | 19 | 27804309 | Cpt1a | carnitine palmitoyltransferase 1, | IPR000542 // | — |
| | | | | liver | Acyltransferase | |
| | | | | | ChoActase/COT/CPT | |
| 93365_s_at | 19 | | 2410174K12Rik | RIKEN cDNA 2410174K12 gene | IPR001440 // TPR | TPR // TPR |
| | | | | | repeat | Domain; 3.2e−10 |
| 93435_at | 19 | 6753572 | Cyp24 | cytochrome P450, 24 | IPR001128 // | p450 // Cytochrome |
| | | | | | Cytochrome P450 /// | P450; 3.2e−102 |
| | | | | | IPR002401 // E-class | |
| | | | | | P450, group I | |
| 93595_at | 19 | 6753448 | Cln2 | ceroid-lipofuscinosis, neuronal 2 | — | — |
| 93671_at | 19 | | Erf | Est2 repressor factor | — | Ets // Ets- |
| | | | | | | domain; 1.1e−54 |
| 93760_at | 19 | | Cript-pending | postsynaptic protein Cript | — | — |
| 94418_at | 19 | | Lce-pending | long chain fatty acyl elongase | IPR002076 // | GNS1_SUR4 // |
| | | | | | GNS1/SUR4 | GNS1/SUR4 |
| | | | | | membrane protein | family; 3.7e−48 |
| 94807_at | 19 | 23943838 | Slc25a1 | solute carrier family 25 | — | mito_carr // |
| | | | | (mitochondrial carrier; citrate | | Mitochondrial carrier |
| | | | | transporter), member 1 | | protein; 1.6e−83 |
| 94906_at | 19 | | Adh1 | alcohol dehydrogenase 1 (class I) | IPR002085 // Zinc- | adh_zinc // Zinc- |
| | | | | | containing alcohol | binding |
| | | | | | dehydrogenase | dehydrogenase; 2.6e−143 |
| | | | | | superfamily /// | |
| | | | | | IPR002328 // Zinc- | |
| | | | | | containing alcohol | |
| | | | | | dehydrogenase | |
| 96910_at | 19 | 22122743 | MGC37245 | hypothetical protein MGC37245 | IPR000873 // AMP- | AMP-binding // AMP- |
| | | | | | dependent | binding enzyme; 7.1e−95 |
| | | | | | synthetase and ligase | |
| 96938_at | 19 | 19482166 | Keg1 | kidney expressed gene 1 | — | — |
| 97257_at | 19 | 21703764 | Cgi-83-pending | CGI-83 protein | IPR001279 // Beta- | lactamase_B // |
| | | | | | lactamase-like | Metallo-beta- |
| | | | | | | lactamase |
| | | | | | | superfamily; 1.9e−23 |
| 97258_at | 19 | 21703764 | Cgi-83-pending | CGI-83 protein | IPR001279 // Beta- | lactamase_B // |
| | | | | | lactamase-like | Metallo-beta- |
| | | | | | | lactamase |
| | | | | | | superfamily; 1.9e−23 |
| 97431_at | 19 | | Slc22a6 | solute carrier family 22 (organic | IPR005828 // General | sugar_tr // Sugar (and |
| | | | | anion transporter), member 6 | substrate transporter | other) |
| | | | | | /// IPR004749 // | transporter; 1.8e−16 |
| | | | | | Organic cation | |
| | | | | | transport protein | |
| 97707_at | 19 | | — | ESTs, Weakly similar to RIKEN | — | — |
| | | | | cDNA 5730493B19 [Mus | | |
| | | | | musculus] [M. musculus] | | |
| AFFX- | 19 | 6679237 | | | | |
| PyruCarbMur/ | | | | | | |
| L09192_5_at | | | | | | |
| 100285_at | 18 | | Col4a3 | procollagen, type IV, alpha 3 | IPR000504 // RNA- | Collagen // Collagen |
| | | | | | binding region RNP-1 | triple helix repeat (20 |
| | | | | | (RNA recognition | copies); 2e−176 /// C4 |
| | | | | | motif) /// IPR000087 | // C-terminal tandem |
| | | | | | // Collagen triple helix | repeated domain in |
| | | | | | repeat /// IPR001442 | type 4; 3.4e−146 |
| | | | | | // Type 4 procollagen, | |
| | | | | | C-terminal repeat | |
| 101666_at | 18 | | Nr5a1 | nuclear receptor subfamily 5, | IPR001628 // Zn- | hormone_rec // |
| | | | | group A, member 1 | finger, C4-type | Ligand-binding domain |
| | | | | | steroid receptor /// | of nuclear |
| | | | | | IPR000324 // Vitamin | hormone; 2.7e−48 /// |
| | | | | | D receptor /// | hormone_rec // |
| | | | | | IPR001723 // Steroid | Ligand-binding domain |
| | | | | | hormone receptor /// | of nuclear |
| | | | | | IPR000536 // Ligand- | hormone; 2.4e−48 /// |
| | | | | | binding domain of | zf-C4 // Zinc finger, C4 |
| | | | | | nuclear hormone | type (two |
| | | | | | receptor | domains); 3.3e−52 |
| 101757_at | 18 | | Nfe2l1 | nuclear factor, erythroid | IPR004827 // Basic- | — |
| | | | | derived 2, - like 1 | leucine zipper (bZIP) | |
| | | | | | transcription factor | |
| 102329_at | 18 | | Cideb | cell death-inducing DNA | IPR003508 // | CIDE-N // CIDE-N |
| | | | | fragmentation factor, alpha | Caspase-activated | domain; 9.6e−46 |
| | | | | subunit-like effector B | nuclease CIDE-N | |
| 103647_at | 18 | | Glb1 | galactosidase, beta 1 | IPR001944 // | Glyco_hydro_35 // |
| | | | | | Glycoside hydrolase, | Glycosyl hydrolases |
| | | | | | family 35 | family 35; 0 |
| 104184_at | 18 | | Nppb | natriuretic peptide precursor type B | — | ANP // Atrial natriuretic |
| | | | | | | peptide; 3.9e−29 |
| 104605_at | 18 | | 1110001I14Rik | RIKEN cDNA 1110001I14 gene | — | — |
| 104748_s_at | 18 | 6678001 | Slc1a1 | solute carrier family 1, member 1 | IPR001991 // | SDF // |
| | | | | | Sodium: dicarboxylate | Sodium: dicarboxylate |
| | | | | | symporter | symporter family; 2.7e−248 |
| 92407_at | 18 | | Myom1 | myomesin 1 | IPR003600 // | ig // Immunoglobulin |
| | | | | | Immunoglobulin-like | domain; 1.2e−22 /// fn3 |
| | | | | | /// IPR000097 // AP | // Fibronectin type III |
| | | | | | endonuclease, family | domain; 3e−100 |
| | | | | | 1 /// IPR003961 // | |
| | | | | | Fibronectin, type III /// | |
| | | | | | IPR003962 // | |
| | | | | | Fibronectin, type III | |
| | | | | | repeat /// IPR003598 | |
| | | | | | // Immunoglobulin C- | |
| | | | | | 2 type /// IPR003006 | |
| | | | | | // | |
| | | | | | Immunoglobulin/major | |
| | | | | | histocompatibility | |
| | | | | | complex | |
| 92600_f_at | 18 | | Cyp4a10 | cytochrome P450, 4a10 | IPR001230 // Prenyl | — |
| | | | | | group binding site | |
| | | | | | (CAAX box) /// | |
| | | | | | IPR002402 // E-class | |
| | | | | | P450, group II /// | |
| | | | | | IPR001128 // | |
| | | | | | Cytochrome P450 /// | |
| | | | | | IPR002401 // E-class | |
| | | | | | P450, group I | |
| 93500_at | 18 | | Alas1 | aminolevulinic acid synthase 1 | IPR001917 // | aminotran_1_2 // |
| | | | | | Aminotransferase, | Aminotransferase |
| | | | | | class-II /// IPR003408 | class I and II; 6.3e−59 |
| | | | | | // Aminolevulinic acid | /// ALA_synthase // |
| | | | | | synthase /// | Aminolevulinic acid |
| | | | | | IPR004839 // | synthase domain; 1.3e−45 |
| | | | | | Aminotransferase, | |
| | | | | | class I and II | |
| 93603_at | 18 | | Mrpl40 | mitochondrial ribosomal protein | — | — |
| | | | | L40 | | |
| 93776_at | 18 | | 1500001L15Rik | RIKEN cDNA 1500001L15 gene | — | — |
| 93868_at | 18 | | Nsdhl | NAD(P) dependent steroid | IPR002225 // 3-beta | 3Beta_HSD // 3-beta |
| | | | | dehydrogenase-like | hydroxysteroid | hydroxysteroid |
| | | | | | dehydrogenase/isomerase | dehydrogenase/isomera; |
| | | | | | | 4.4e−95 |
| 93933_at | 18 | | Ppp1r3c | protein phosphatase 1, regulatory | IPR005036 // | — |
| | | | | (inhibitor) subunit 3C | Putative phosphatase | |
| | | | | | regulatory subunit | |
| 94330_at | 18 | | Npl | N-acetylneuraminate pyruvate | IPR002220 // | DHDPS // |
| | | | | lyase | Dihydrodipicolinate | Dihydrodipicolinate |
| | | | | | synthetase | synthetase |
| | | | | | | family; 4.5e−30 |
| 95000_g_at | 18 | | Cubn | cubilin (intrinsic factor-cobalamin | IPR001412 // | — |
| | | | | receptor) | Aminoacyl-tRNA | |
| | | | | | synthetase, class I /// | |
| | | | | | IPR000859 // CUB | |
| | | | | | domain | |
| 95066_at | 18 | | Taldo1 | transaldolase 1 | IPR004730 // | — |
| | | | | | Transaldolase AB /// | |
| | | | | | IPR001585 // | |
| | | | | | Transaldolase | |
| 96077_at | 18 | | Slc17a1 | solute carrier family 17 vesicular | IPR005828 // General | — |
| | | | | glutamate transporter), member 1 | substrate transporter | |
| | | | | | /// IPR004745 // | |
| | | | | | Na(+)-dependent | |
| | | | | | inorganic phosphate | |
| | | | | | cotransporter | |
| 97172_s_at | 18 | | Abcc9 | ATP-binding cassette, sub-family | IPR003439 // ABC | ABC_tran // ABC |
| | | | | C (CFTR/MRP), member 9 | transporter /// | transporter; 9.5e−87 /// |
| | | | | | IPR000388 // | ABC_membrane // |
| | | | | | Sulphonylurea | ABC transporter |
| | | | | | receptor /// | transmembrane |
| | | | | | IPR003593 // AAA | region; 2.7e−68 /// |
| | | | | | ATPase /// | ABC_tran // ABC |
| | | | | | IPR001140 // ABC | transporter; 2.1e−87 /// |
| | | | | | transporter, | ABC_tran // ABC |
| | | | | | transmembrane | transporter; 1.4e−89 |
| | | | | | region /// IPR001475 | |
| | | | | | // Sulphonylurea | |
| | | | | | receptor, type 2 | |
| 97281_at | 18 | | AA420407 | expressed sequence AA420407 | IPR002618 // UTP- | UDPGP // UTP- |
| | | | | | glucose-1-phosphate | glucose-1-phosphate |
| | | | | | uridylyltransferase | uridylyltransferase; |
| | | | | | | 1.3e−234 |
| 97477_at | 18 | 7305579 | Timm8b | translocase of inner mitochondrial | IPR004217 // Zn- | zf-Tim10_DDP // |
| | | | | membrane 8 homolog b (yeast) | finger, Tim10/DDP | Tim10/DDP family zinc |
| | | | | | type | finger; 3.2e−28 |
| 97521_at | 18 | | Ass1 | argininosuccinate synthetase 1 | IPR001518 // | Arginosuc_synth // |
| | | | | | Argininosuccinate | Arginosuccinate |
| | | | | | synthase | synthase; 2.3e−262 |
| 97751_f_at | 18 | | — | ESTs, Moderately similar to | — | — |
| | | | | G3P_MOUSE Glyceraldehyde 3- | | |
| | | | | phosphate dehydrogenase | | |
| | | | | (GAPDH) [M. musculus] | | |
| 98626_at | 18 | | 1810017G16Rik | RIKEN cDNA 1810017G16 gene | — | — |
| 99184_at | 18 | | Csad | cysteine sulfinic acid | — | pyridoxal_deC // |
| | | | | decarboxylase | | Pyridoxal-dependent |
| | | | | | | decarboxylase |
| | | | | | | conse; 1.4e−125 |
| 99580_s_at | 18 | | Ugt1a1 | UDP-glucuronosyltransferase 1 | IPR002213 // UDP- | — |
| | | | | family, member 1 | glucoronosyl/UDP- | |
| | | | | | glucosyl transferase | |
| 100573_f_at | 17 | | Gpi1 | glucose phosphate isomerase 1 | IPR001672 // | PGI // |
| | | | | | Phosphoglucose | Phosphoglucose |
| | | | | | isomerase (PGI) | isomerase; 0 |
| 101695_at | 17 | | Eif3s6 | eukaryotic translation initiation | IPR000717 // Domain | — |
| | | | | factor 3, subunit 6 | in components of the | |
| | | | | | proteasome, COP9- | |
| | | | | | complex and elF3 | |
| | | | | | (PCI) | |
| 101822_at | 17 | | Mc3r | melanocortin 3 receptor | — | 7tm_1 // 7 |
| | | | | | | transmembrane |
| | | | | | | receptor (rhodopsin |
| | | | | | | family); 2.4e−54 |
| 103484_at | 17 | | Pop3-pending | popeye 3 | — | — |
| 103702_i_at | 17 | | C730048C13Rik | RIKEN cDNA C730048C13 gene | — | — |
| 103833_at | 17 | | Hipk2 | homeodomain interacting protein | IPR001245 // | pkinase // Protein |
| | | | | kinase 2 | Tyrosine protein | kinase domain; 1.3e−49 |
| | | | | | kinase /// IPR002290 | |
| | | | | | // Serine/Threonine | |
| | | | | | protein kinase /// | |
| | | | | | IPR000719 // | |
| | | | | | Eukaryotic protein | |
| | | | | | kinase | |
| 103899_at | 17 | | 4930558F19Rik | RIKEN cDNA 4930558F19 gene | — | — |
| 104438_at | 17 | | Zfp30 | zinc finger protein 30 | IPR001909 // KRAB | zf-C2H2 // Zinc finger, |
| | | | | | box /// IPR000822 // | C2H2 type; 8e−80 /// |
| | | | | | Zn-finger, C2H2 type | KRAB // KRAB |
| | | | | | | box; 5.6e−23 |
| 92650_at | 17 | | Man1b | mannosidase 1, beta | IPR001382 // | Glyco_hydro_47 // |
| | | | | | Glycoside hydrolase, | Glycosyl hydrolase |
| | | | | | family 47 | family 47; 6.8e−286 |
| 92829_at | 17 | 6680309 | Hspe1 | heat shock protein 1 (chaperonin | IPR001476 // | cpn10 // Chaperonin |
| | | | | 10) | Chaperonin Cpn10 | 10 Kd subunit; 2.8e−46 |
| 93798_at | 17 | | Atp1a1 | ATPase, Na+/K+ transporting, | IPR004014 // Cation | Cation_ATPase_N // |
| | | | | alpha 1 polypeptide | transporting ATPase, | Cation |
| | | | | | N terminal /// | transporter/ATPase, |
| | | | | | IPR001757 // | N-terminus; 1.1e−37 /// |
| | | | | | ATPase, E1-E2 type | Hydrolase // haloacid |
| | | | | | /// IPR006069 // | dehalogenase-like |
| | | | | | Cation transporting | hydrolase; 4.2e−15 /// |
| | | | | | ATPase /// | E1-E2_ATPase // E1- |
| | | | | | IPR005834 // | E2 ATPase; 1.3e−113 |
| | | | | | haloacid | /// Cation_ATPase_C |
| | | | | | dehalogenase-like | // Cation transporting |
| | | | | | hydrolase /// | ATPase, C- |
| | | | | | IPR005775 // Na+/K+ | terminu; 1.3e−68 |
| | | | | | ATPase, alpha | |
| | | | | | subunit /// IPR006068 | |
| | | | | | // Cation transporting | |
| | | | | | ATPase, C-terminal | |
| 94262_at | 17 | | B230333E16Rik | RIKEN cDNA B230333E16 gene | — | — |
| 96336_at | 17 | 13385454 | Gatm | glycine amidinotransferase (L- | IPR003198 // | Amidinotransf // |
| | | | | arginine:glycine | Amidinotransferase | Amidinotransferase; |
| | | | | amidinotransferase) | /// IPR000531 // | 3.6e−06 |
| | | | | | TonB-dependent | |
| | | | | | receptor protein | |
| 96918_at | 17 | | Fbp1 | fructose bisphosphatase 1 | IPR000146 // Inositol | FBPase // Fructose-1- |
| | | | | | phosphatase/fructose- | 6- |
| | | | | | 1,6-bisphosphatase | bisphosphatase; 4.4e−197 |
| 97515_at | 17 | 31982273 | Hsd17b4 | hydroxysteroid (17-beta) | IPR002539 // MaoC- | SCP2 // SCP-2 sterol |
| | | | | dehydrogenase 4 | like dehydratase /// | transfer family; 7.9e−48 |
| | | | | | IPR002198 // Short- | /// MaoC_dehydratas // |
| | | | | | chain | MaoC like |
| | | | | | dehydrogenase/reductase | domain; 1.3e−50 /// |
| | | | | | SDR /// | adh_short // short |
| | | | | | IPR003033 // Sterol- | chain |
| | | | | | binding /// IPR002347 | dehydrogenase; 2.4e−65 |
| | | | | | // Glucose/ribitol | |
| | | | | | dehydrogenase | |
| 97758_at | 17 | | Prdx1 | peroxiredoxin 1 | IPR000866 // Alkyl | AhpC-TSA // |
| | | | | | hydroperoxide | AhpC/TSA family; 8e−89 |
| | | | | | reductase/Thiol | |
| | | | | | specific antioxidant/ | |
| | | | | | Mal allergen | |
| 97926_s_at | 17 | | Pparg | peroxisome proliferator activated | IPR001628 // Zn- | hormone_rec // |
| | | | | receptor gamma | finger, C4-type | Ligand-binding domain |
| | | | | | steroid receptor /// | of nuclear |
| | | | | | IPR003077 // | hormone; 7.7e−40 /// |
| | | | | | Peroxisome | zf-C4 // Zinc finger, C4 |
| | | | | | proliferator-activated | type (two |
| | | | | | receptor, gamma /// | domains); 2.3e−45 |
| | | | | | IPR003074 // | |
| | | | | | Peroxisome | |
| | | | | | proliferator-activated | |
| | | | | | receptor /// | |
| | | | | | IPR001723 // Steroid | |
| | | | | | hormone receptor /// | |
| | | | | | IPR000536 // Ligand- | |
| | | | | | binding domain of | |
| | | | | | nuclear hormone | |
| | | | | | receptor | |
| 98322_at | 17 | | Slc22a5 | solute carrier family 22 (organic | IPR005829 // Sugar | sugar_tr // Sugar (and |
| | | | | cation transporter), member 5 | transporter | other) |
| | | | | | superfamily /// | transporter; 1.4e−07 |
| | | | | | IPR005828 // General | |
| | | | | | substrate transporter | |
| | | | | | /// IPR004749 // | |
| | | | | | Organic cation | |
| | | | | | transport protein | |
| 98496_at | 17 | | Gys3 | glycogen synthase 3, brain | — | — |
| 98552_at | 17 | | 2600009M07Rik | RIKEN cDNA 2600009M07 gene | — | — |
| 99587_at | 17 | | Rab7 | RAB7, member RAS oncogene | IPR002078 // Sigma- | ras // Ras family; 6.3e−94 |
| | | | | family | 54 factor interaction | |
| | | | | | domain /// IPR005225 | |
| | | | | | // Small GTP-binding | |
| | | | | | protein domain /// | |
| | | | | | IPR003579 // Ras | |
| | | | | | small GTPase, Rab | |
| | | | | | type /// IPR001806 // | |
| | | | | | Ras GTPase | |
| | | | | | superfamily | |
| 99872_s_at | 17 | | Ftl1 | ferritin light chain 1 | IPR001519 // Ferritin | ferritin // Ferritin-like |
| | | | | | | domain; 2.2e−53 |
| 99973_s_at | 17 | | Kcnj15 | potassium inwardly-rectifying | IPR001622 // K+ | IRK // Inward rectifier |
| | | | | channel, subfamily J, member 15 | channel, pore region | potassium |
| | | | | | /// IPR001838 // K+ | channel; 2.2e−221 |
| | | | | | channel, inward | |
| | | | | | rectifier /// | |
| | | | | | IPR003270 // Kir1.3 | |
| | | | | | inward rectifier K+ | |
| | | | | | channel | |
| 100041_at | 16 | | 3010027G13Rik | RIKEN cDNA 3010027G13 gene | IPR001993 // | mito_carr // |
| | | | | | Mitochondrial | Mitochondrial carrier |
| | | | | | substrate carrier | protein; 1.3e−65 |
| 101013_at | 16 | | Oaz1 | ornithine decarboxylase anitizyme | IPR002993 // | ODC_AZ // Ornithine |
| | | | | | Ornithine | decarboxylase |
| | | | | | decarboxylase | antizyme; 1.6e−158 |
| | | | | | antizyme | |
| 101913_at | 16 | | — | ESTs, Highly similar to | — | — |
| | | | | CLC5_MOUSE Chloride channel | | |
| | | | | protein 5 (CIC-5) [M. musculus] | | |
| 102899_at | 16 | | Siat7c | sialyltransferase 7 ((alpha-N- | IPR001675 // | Glyco_transf_29 // |
| | | | | acetylneuraminyl 2,3- | Glycosyl transferase, | Glycosyltransferase |
| | | | | betagalactosyl-1,3)-N-acetyl | family 29 | family 29 (sialyl; 1.2e−104 |
| | | | | galactosaminide alpha-2,6- | | |
| | | | | sialyltransferase) C | | |
| 104014_at | 16 | | Hfe | hemochromatosis | IPR001039 // Major | ig // Immunoglobulin |
| | | | | | histocompatibility | domain; 8.8e−05 /// |
| | | | | | complex protein, | MHC_I // Class I |
| | | | | | class I /// IPR003006 | Histocompatibility |
| | | | | | // | antigen, |
| | | | | | Immunoglobulin/major | domains; 5.4e−49 |
| | | | | | histocompatibility | |
| | | | | | complex /// | |
| | | | | | IPR003597 // | |
| | | | | | Immunoglobulin C- | |
| | | | | | type | |
| 104101_at | 16 | | 1200006P13Rik | RIKEN cDNA 1200006P13 gene | IPR004709 // | Na_H_Exchanger // |
| | | | | | Sodium/hydrogen | Sodium/hydrogen |
| | | | | | exchanger subfamily | exchanger family; 1.5e−103 |
| | | | | | /// IPR006153 // | |
| | | | | | Sodium/hydrogen | |
| | | | | | exchanger | |
| 104745_at | 16 | | Arl6ip2 | ADP-ribosylation-like factor 6 | — | — |
| | | | | interacting protein 2 | | |
| 93051_at | 16 | | Ephx2 | epoxide hydrolase 2, cytoplasmic | IPR005833 // | abhydrolase // |
| | | | | | Haloacid | alpha/beta hydrolase |
| | | | | | dehalogenase/epoxide | fold; 8.2e−50 /// |
| | | | | | hydrolase /// | Hydrolase // haloacid |
| | | | | | IPR000073 // | dehalogenase-like |
| | | | | | Alpha/beta hydrolase | hydrolase; 2.3e−16 |
| | | | | | fold /// IPR003089 // | |
| | | | | | Alpha/beta hydrolase | |
| | | | | | /// IPR005834 // | |
| | | | | | haloacid | |
| | | | | | dehalogenase-like | |
| | | | | | hydrolase /// | |
| | | | | | IPR000639 // | |
| | | | | | Epoxide hydrolase /// | |
| | | | | | IPR000379 // | |
| | | | | | Esterase/lipase/thioesterase, | |
| | | | | | active site | |
| 94042_f_at | 16 | | Gng5 | guanine nucleotide binding protein | IPR001770 // G- | — |
| | | | | (G protein), gamma 5 subunit | protein, gamma | |
| | | | | | subunit | |
| 94057_g_at | 16 | | Scd1 | stearoyl-Coenzyme A desaturase 1 | IPR001522 // Fatty | FA_desaturase // Fatty |
| | | | | | acid desaturase, type | acid desaturase; 5.2e−80 |
| | | | | | 1 /// IPR005804 // | |
| | | | | | Fatty acid desaturase | |
| | | | | | family | |
| 94276_at | 16 | | Hsd17b12 | hydroxysteroid (17-beta) | IPR002198 // Short- | adh_short // short |
| | | | | dehydrogenase 12 | chain | chain |
| | | | | | dehydrogenase/reductase | dehydrogenase; 1.7e−37 |
| | | | | | SDR /// | |
| | | | | | IPR002347 // | |
| | | | | | Glucose/ribitol | |
| | | | | | dehydrogenase | |
| 95518_at | 16 | | 1810015C04Rik | RIKEN cDNA 1810015C04 gene | — | — |
| 96068_at | 16 | | 1500034J20Rik | RIKEN cDNA 1500034J20 gene | IPR000508 // Signal | Peptidase_S26 // |
| | | | | | peptidase /// | Signal peptidase |
| | | | | | IPR000223 // | I; 7.7e−06 |
| | | | | | Bacterial signal | |
| | | | | | peptidase S26A | |
| 96346_at | 16 | | Cdo1 | cysteine dioxygenase 1, cytosolic | — | — |
| 97402_at | 16 | | Temt | thioether S-methyltransferase | IPR000940 // | NNMT_PNMT_TEMT |
| | | | | | Methyltransferase, | // NNMT/PNMT/TEMT |
| | | | | | NNMT/PNMT/TEMT | family; 2.6e−176 |
| | | | | | family /// IPR001601 | |
| | | | | | // Generic | |
| | | | | | methyltransferase | |
| 97450_s_at | 16 | 20070418 | Aldh7a1 | aldehyde dehydrogenase family 7, | IPR002086 // | aldedh // Aldehyde |
| | | | | member A1 | Aldehyde | dehydrogenase |
| | | | | | dehydrogenase | family; 9.5e−166 |
| 97800_at | 16 | | Fastk | Fas-activated serine/threonine | — | — |
| | | | | kinase | | |
| 100424_at | 15 | | Ercc1 | excision repair cross- | IPR000445 // Helix- | HHH // Helix-hairpin- |
| | | | | complementing rodent repair | hairpin-helix motif /// | helix motif; 1.5e−09 /// |
| | | | | deficiency, complementation | IPR003583 // Helix- | Rad10 // DNA repair |
| | | | | group 1 | hairpin-helix DNA- | protein rad10; 3.5e−47 |
| | | | | | binding, class 1 /// | |
| | | | | | IPR004579 // DNA | |
| | | | | | repair protein rad10 | |
| 100597_at | 15 | | Gyg1 | glycogenin 1 | IPR002495 // | Glyco_transf_8 // |
| | | | | | Glycosyl transferase, | Glycosyl transferase |
| | | | | | family 8 | family 8; 0.00077 |
| 100959_at | 15 | | S100a13 | S100 calcium binding protein A13 | IPR002048 // | S_100 // S-100/ICaBP |
| | | | | | Calcium-binding EF- | type calcium binding |
| | | | | | hand /// IPR001751 // | domain; 2.7e−13 |
| | | | | | Calcium-binding | |
| | | | | | protein, S-100/ICaBP | |
| | | | | | type | |
| 102041_at | 15 | | Myom2 | myomesin 2 | IPR003600 // | fn3 // Fibronectin type |
| | | | | | Immunoglobulin-like | III domain; 1.7e−105 /// |
| | | | | | /// IPR003961 // | ig // Immunoglobulin |
| | | | | | Fibronectin, type III /// | domain; 4e−21 |
| | | | | | IPR003962 // | |
| | | | | | Fibronectin, type III | |
| | | | | | repeat /// IPR003598 | |
| | | | | | // Immunoglobulin C- | |
| | | | | | 2 type /// IPR003006 | |
| | | | | | // | |
| | | | | | Immunoglobulin/major | |
| | | | | | histocompatibility | |
| | | | | | complex | |
| 102671_at | 15 | | Creb1 | cAMP responsive element binding | IPR004827 // Basic- | pKID // pKID |
| | | | | protein 1 | leucine zipper (bZIP) | domain; 4.7e−24 /// |
| | | | | | transcription factor /// | bZIP // bZIP |
| | | | | | IPR001630 // cAMP | transcription |
| | | | | | response element | factor; 6.4e−20 /// bZIP |
| | | | | | binding (CREB) | // bZIP transcription |
| | | | | | protein /// IPR003102 | factor; 7.2e−21 |
| | | | | | // Coactivator CBP, | |
| | | | | | pKID | |
| 103845_at | 15 | | Slc31a1 | solute carrier family 31, member 1 | — | — |
| 92726_at | 15 | | Sox6 | SRY-box containing gene 6 | IPR000910 // | HMG_box // HMG |
| | | | | | HMG1/2 (high | (high mobility group) |
| | | | | | mobility group) box | box; 9e−27 |
| 92775_at | 15 | | Pabpc4 | poly(A) binding protein, | IPR002004 // Poly- | rrm // RNA recognition |
| | | | | cytoplasmic 4 (inducible form) | adenylate-binding | motif. (a.k.a. RRM, |
| | | | | | protein/HECT- | RBD, or; 3.5e−111 /// |
| | | | | | associated /// | PABP // Poly- |
| | | | | | IPR000504 // RNA- | adenylate binding |
| | | | | | binding region RNP-1 | protein, unique |
| | | | | | (RNA recognition | domai; 2.3e−45 |
| | | | | | motif) | |
| 94012_at | 15 | 7305575 | Timm13a | translocase of inner mitochondrial | IPR004217 // Zn- | zf-Tim10_DDP // |
| | | | | membrane 13 homolog a (yeast) | finger, Tim10/DDP | Tim10/DDP family zinc |
| | | | | | type | finger; 2.7e−25 |
| 94056_at | 15 | | Scd1 | stearoyl-Coenzyme A desaturase 1 | IPR001522 // Fatty | FA_desaturase // Fatty |
| | | | | | acid desaturase, type | acid desaturase; 5.2e−80 |
| | | | | | 1 /// IPR005804 // | |
| | | | | | Fatty acid desaturase | |
| | | | | | family | |
| 94922_i_at | 15 | | 4930431L18Rik | RIKEN cDNA 4930431L18 gene | — | — |
| 95026_at | 15 | | 0610039N19Rik | RIKEN cDNA 0610039N19 gene | — | — |
| 95407_at | 15 | | Pah | phenylalanine hydroxylase | IPR002912 // Amino | biopterin_H // |
| | | | | | acid-binding ACT /// | Biopterin-dependent |
| | | | | | IPR001273 // | aromatic amino acid |
| | | | | | Aromatic amino acid | h; 3.7e−294 /// ACT // |
| | | | | | hydroxylase /// | ACT domain; 5.5e−11 |
| | | | | | IPR005961 // | |
| | | | | | Phenylalanine-4- | |
| | | | | | hydroxylase, | |
| | | | | | tetrameric form | |
| 96934_at | 15 | | 1110002M09Rik | RIKEN cDNA 1110002M09 gene | — | — |
| 97334_at | 15 | | Hes6 | hairy and enhancer of split 6, | IPR003650 // Orange | HLH // Helix-loop-helix |
| | | | | (Drosophila) | /// IPR001092 // Basic | DNA-binding |
| | | | | | helix-loop-helix | domain; 8.3e−09 |
| | | | | | dimerization domain | |
| | | | | | bHLH | |
| 97449_at | 15 | | Aldh7a1 | aldehyde dehydrogenase family 7, | IPR002086 // | aldedh // Aldehyde |
| | | | | member A1 | Aldehyde | dehydrogenase |
| | | | | | dehydrogenase | family; 9.5e−166 |
| 98447_at | 15 | | Cebpa | CCAAT/enhancer binding protein | IPR004827 // Basic- | — |
| | | | | (C/EBP), alpha | leucine zipper (bZIP) | |
| | | | | | transcription factor | |
| 98871_at | 15 | | Oa1 | mouse homolog of human ocular | IPR001414 // Ocular | Ocular_alb // Ocular |
| | | | | albinism 1 (Nettleship-Falls) | albinism protein, type 1 | albinism type 1 |
| | | | | | | protein; 0 |
| 99056_at | 15 | | Pcbd | 6-pyruvoyl-tetrahydropterin | IPR001533 // | Pterin_4a // Pterin 4 |
| | | | | synthase/dimerization cofactor of | Transcriptional | alpha carbinolamine |
| | | | | hepatocyte nuclear factor 1 alpha | coactivator/pterin | dehydratase; 6.4e−61 |
| | | | | (TCF1) | dehydratase | |
| 99164_at | 15 | | Mapbpip- | mitogen activated protein binding | IPR004942 // | Robl_LC7 // |
| | | | pending | protein interacting protein | Roadblock/LC7 | Roadblock/LC7 |
| | | | | | family | domain; 2e−25 |
| 99988_at | 15 | | 4933427L07Rik | RIKEN cDNA 4933427L07 gene | — | — |

Table 8 shows motifs associated with differential expression on days 1, 2, and 3.

| | | | Nominal P- | Adjusted | | |
|---|---|---|---|---|---|---|
| Day | Motif | Frequency | value | P-value | Annotation | Reference |
| 1 | TGACCTTG | 0.07 | 3.15E−11 | 2.06E−06 | Errα | (22) |
| | TGACCTTGA | 0.02 | 4.59E−10 | 1.20E−04 | Errα | |
| 2 | TGACCTTG | 0.07 | 4.44E−14 | 2.91E−09 | Errα | (22) |
| | TGACCTT | 0.16 | 3.62E−12 | 5.93E−08 | Errα | |
| | TGACCT | 0.45 | 1.46E−11 | 5.97E−08 | NR half-site | (35) |
| | GACCTTG | 0.16 | 7.92E−11 | 1.30E−06 | Errα | |
| | GACCTT | 0.41 | 1.42E−09 | 5.81E−06 | Errα | |
| | TTGACC | 0.27 | 2.42E−07 | 9.92E−04 | Errα | |
| 3 | CTTCCG | 0.33 | 2.19E−12 | 8.97E−09 | Gabpa | (36) |
| | TGACCTTG | 0.07 | 1.17E−11 | 7.66E−07 | Errα | (22) |
| | TGACCTT | 0.16 | 1.23E−10 | 2.02E−06 | Errα | |
| | CCCGCC | 0.54 | 2.04E−08 | 8.36E−05 | | |
| | GCGGCG | 0.43 | 3.78E−08 | 1.55E−04 | | |
| | AGGTCA | 0.42 | 3.90E−08 | 1.60E−04 | NR half-site | (35) |
| | CTTCCGG | 0.16 | 1.95E−08 | 3.19E−04 | Gabpa | |
| | TTCCGG | 0.31 | 1.09E−07 | 4.46E−04 | Gabpa | |
| | GGGGCG | 0.54 | 1.24E−07 | 5.08E−04 | | |
| | TTCCGCT | 0.07 | 3.30E−08 | 5.41E−04 | Gabpa | |
| | GCCGGC | 0.42 | 1.57E−07 | 6.44E−04 | | |
| | ACTTCCG | 0.09 | 5.11E−08 | 8.38E−04 | Gabpa | |

motifADE was performed using the mouse promoter database on each of days 1, 2, and 3. All motifs achieving a Bonferroni-corrected P-value < 1 × 10⁻³ are shown. Annotations of the motif and the literature references, when available, are indicated.

TABLE 9
motifs discovered using the mouse promoter database achieving
P < 0.05

| | | | | Adjusted P- |
|---|---|---|---|---|
| Day | Motif | Frequency | P-value | value |
| 1 | TGACCTTG | 0.07 | 3.15E−11 | 2.06E−06 |
| | TGACCTTGA | 0.02 | 4.59E−10 | 1.20E−04 |
| | GACCTTGA | 0.05 | 5.76E−08 | 3.77E−03 |
| | GACCTTG | 0.16 | 1.54E−06 | 2.53E−02 |
| | GTCACG | 0.18 | 8.04E−06 | 3.29E−02 |
| 2 | TGACCTTG | 0.07 | 4.44E−14 | 2.91E−09 |
| | TGACCTT | 0.16 | 3.62E−12 | 5.93E−08 |
| | TGACCT | 0.45 | 1.46E−11 | 5.97E−08 |
| | GACCTTG | 0.16 | 7.92E−11 | 1.30E−06 |
| | GACCTT | 0.41 | 1.42E−09 | 5.81E−06 |
| | TTGACC | 0.27 | 2.42E−07 | 9.92E−04 |
| | GTGACCTT | 0.05 | 3.86E−08 | 2.53E−03 |
| | GTGACCT | 0.15 | 3.91E−07 | 6.41E−03 |
| | GTGACCTTG | 0.02 | 3.97E−08 | 1.04E−02 |
| | TGACCTTGA | 0.02 | 4.63E−08 | 1.21E−02 |
| | AGGTCA | 0.42 | 3.46E−06 | 1.42E−02 |
| | CGCTGAGG | 0.04 | 3.06E−07 | 2.01E−02 |
| | GACCTTGA | 0.05 | 3.33E−07 | 2.19E−02 |
| | AGGTCAC | 0.13 | 1.99E−06 | 3.26E−02 |
| | GTGACC | 0.40 | 8.80E−06 | 3.61E−02 |
| 3 | CTTCCG | 0.33 | 2.19E−12 | 8.97E−09 |
| | TGACCTTG | 0.07 | 1.17E−11 | 7.66E−07 |
| | TGACCTT | 0.16 | 1.23E−10 | 2.02E−06 |
| | CCCGCC | 0.54 | 2.04E−08 | 8.36E−05 |
| | GCGGCG | 0.43 | 3.78E−08 | 1.55E−04 |
| | AGGTCA | 0.42 | 3.90E−08 | 1.60E−04 |
| | CTTCCGG | 0.16 | 1.95E−08 | 3.19E−04 |
| | TTCCGG | 0.31 | 1.09E−07 | 4.46E−04 |
| | GGGGCG | 0.54 | 1.24E−07 | 5.08E−04 |
| | TTCCGCT | 0.07 | 3.30E−08 | 5.41E−04 |
| | GCCGGC | 0.42 | 1.57E−07 | 6.44E−04 |
| | ACTTCCG | 0.09 | 5.11E−08 | 8.38E−04 |
| | GACCTT | 0.41 | 2.72E−07 | 1.11E−03 |
| | CGGGGC | 0.51 | 4.86E−07 | 1.99E−03 |
| | ATGGCGGC | 0.05 | 4.76E−08 | 3.12E−03 |
| | GACCTTG | 0.16 | 1.90E−07 | 3.12E−03 |
| | CTTCCGGC | 0.05 | 7.34E−08 | 4.81E−03 |
| | ATGGCGG | 0.11 | 3.24E−07 | 5.31E−03 |
| | AAGATGGCG | 0.03 | 2.07E−08 | 5.43E−03 |
| | CCGGGG | 0.47 | 1.43E−06 | 5.85E−03 |
| | GCGGAC | 0.24 | 1.52E−06 | 6.23E−03 |
| | GGCGGC | 0.48 | 1.55E−06 | 6.35E−03 |
| | TCACGG | 0.19 | 1.79E−06 | 7.31E−03 |
| | GTGACCTT | 0.05 | 1.23E−07 | 8.07E−03 |
| | CCGGCT | 0.39 | 2.23E−06 | 9.13E−03 |
| | GGCCGG | 0.47 | 2.24E−06 | 9.16E−03 |
| | TCACCG | 0.21 | 2.79E−06 | 1.14E−02 |
| | GCCGGG | 0.49 | 2.81E−06 | 1.15E−02 |
| | CGCCTT | 0.30 | 2.93E−06 | 1.20E−02 |
| | CGGACC | 0.24 | 3.33E−06 | 1.36E−02 |
| | TTCCGC | 0.23 | 3.42E−06 | 1.40E−02 |
| | CGCTGA | 0.26 | 3.44E−06 | 1.41E−02 |
| | CCCCGC | 0.51 | 3.55E−06 | 1.46E−02 |
| | CGCGAG | 0.24 | 3.71E−06 | 1.52E−02 |
| | GTCACG | 0.18 | 4.14E−06 | 1.69E−02 |
| | CGTCCT | 0.25 | 4.15E−06 | 1.70E−02 |
| | AAGGTCA | 0.15 | 1.28E−06 | 2.10E−02 |
| | GCCCGG | 0.49 | 5.14E−06 | 2.11E−02 |
| | CCGCCG | 0.36 | 5.25E−06 | 2.15E−02 |
| | TCCGGG | 0.42 | 5.75E−06 | 2.35E−02 |
| | AAGATGGC | 0.08 | 3.93E−07 | 2.57E−02 |
| | GGCGGA | 0.40 | 6.56E−06 | 2.69E−02 |
| | GGGCGG | 0.58 | 7.63E−06 | 3.12E−02 |
| | CGGGCG | 0.38 | 7.77E−06 | 3.18E−02 |
| | ACCCCG | 0.31 | 8.07E−06 | 3.30E−02 |
| | CGCGCC | 0.37 | 8.13E−06 | 3.33E−02 |
| | CGCCTC | 0.41 | 9.12E−06 | 3.74E−02 |
| | TTCCCG | 0.34 | 9.44E−06 | 3.86E−02 |
| | GGGTCGTGG | 0.01 | 1.56E−07 | 4.09E−02 |
| | CGGCGG | 0.40 | 1.01E−05 | 4.15E−02 |
| | CCGGAA | 0.30 | 1.14E−05 | 4.68E−02 |
| | CGTCGC | 0.16 | 1.15E−05 | 4.73E−02 |

motif ADE was performed using the mouse promoter database on each of days 1, 2, and 3. Motifs achieving a Bonferroni corrected P value <0.05 are shown. Motif ADE was performed using the mouse promoter database on each of days 1, 2, and 3. Motifs achieving a Bonferroni corrected P

Table 10 shows motifs discovered using the masked promoter database achieving P<0.05,

| Day | Motif | Frequency | P-value | Adjusted P-value |
|---|---|---|---|---|
| 1 | TGACCTTG | 0.04 | 7.30E−11 | 4.78E−06 |
| | TGACCTT | 0.09 | 2.65E−07 | 4.34E−03 |
| | AAGGTC | 0.20 | 7.83E−06 | 3.21E−02 |
| | CTTCCGG | 0.12 | 2.56E−06 | 4.20E−02 |
| 2 | TGACCT | 0.26 | 1.43E−13 | 5.84E−10 |
| | TGACCTT | 0.09 | 1.74E−12 | 2.85E−08 |
| | TGACCTTG | 0.04 | 2.59E−09 | 1.70E−04 |
| | GACCTT | 0.23 | 4.88E−08 | 2.00E−04 |
| | GTGACCTT | 0.03 | 3.23E−09 | 2.12E−04 |
| | GTGACCT | 0.09 | 1.58E−08 | 2.59E−04 |
| | AGGTCA | 0.25 | 2.04E−07 | 8.37E−04 |
| | GACCTTG | 0.08 | 7.65E−08 | 1.25E−03 |
| | GTGACCTTG | 0.02 | 3.02E−08 | 7.93E−03 |
| | GGTCAC | 0.24 | 2.00E−06 | 8.17E−03 |
| | ACCTTG | 0.22 | 2.05E−06 | 8.38E−03 |
| | AGGTCAC | 0.08 | 8.57E−07 | 1.40E−02 |
| | TTTTCGT | 0.02 | 1.96E−06 | 3.22E−02 |
| 3 | TGACCTT | 0.09 | 7.77E−16 | 1.27E−11 |
| | CTTCCG | 0.25 | 7.59E−14 | 3.11E−10 |
| | TGACCTTG | 0.04 | 8.68E−13 | 5.69E−08 |
| | GTGACCTT | 0.03 | 8.75E−13 | 5.74E−08 |
| | CTTCCGG | 0.12 | 6.12E−12 | 1.00E−07 |
| | GTGACCT | 0.09 | 3.96E−11 | 6.48E−07 |
| | GACCTT | 0.23 | 1.39E−09 | 5.71E−06 |
| | ATGGCGGC | 0.05 | 2.59E−10 | 1.70E−05 |
| | GACCTTG | 0.08 | 1.23E−09 | 2.01E−05 |
| | TTCCGG | 0.24 | 1.79E−08 | 7.34E−05 |
| | CTTCCGGC | 0.04 | 1.66E−09 | 1.09E−04 |
| | TGACCT | 0.26 | 3.58E−08 | 1.47E−04 |
| | CCTTCCG | 0.08 | 1.67E−08 | 2.74E−04 |
| | AAGATGGCG | 0.03 | 1.17E−09 | 3.07E−04 |
| | ATGGCGGCG | 0.03 | 1.28E−09 | 3.37E−04 |
| | CCGGGG | 0.38 | 1.03E−07 | 4.23E−04 |
| | GGCGGG | 0.52 | 1.33E−07 | 5.47E−04 |
| | GTGACCTTG | 0.02 | 4.87E−09 | 1.28E−03 |
| | ACTTCCG | 0.08 | 9.04E−08 | 1.48E−03 |
| | AGATGGCG | 0.04 | 3.79E−08 | 2.48E−03 |
| | ATGGCGG | 0.10 | 1.66E−07 | 2.72E−03 |
| | AGATGGCGG | 0.02 | 1.11E−08 | 2.90E−03 |
| | AGGTCA | 0.25 | 1.04E−06 | 4.25E−03 |
| | CCCGCC | 0.47 | 1.29E−06 | 5.30E−03 |
| | CGGTGA | 0.20 | 1.38E−06 | 5.66E−03 |
| | GGCGGC | 0.43 | 1.55E−06 | 6.34E−03 |
| | GCGGCG | 0.39 | 1.83E−06 | 7.51E−03 |
| | TTCCGCT | 0.05 | 4.87E−07 | 7.98E−03 |
| | GCGTCA | 0.11 | 2.30E−06 | 9.41E−03 |
| | ACTTCCGG | 0.04 | 1.89E−07 | 1.24E−02 |
| | TTCCGC | 0.18 | 3.93E−06 | 1.61E−02 |
| | CGTCCT | 0.17 | 4.00E−06 | 1.64E−02 |
| | CTGCGG | 0.35 | 4.81E−06 | 1.97E−02 |
| | CGGGGC | 0.43 | 4.86E−06 | 1.99E−02 |
| | GCCGGC | 0.33 | 6.24E−06 | 2.56E−02 |
| | CCGGCT | 0.27 | 6.34E−06 | 2.60E−02 |
| | GACCTTCC | 0.03 | 4.71E−07 | 3.09E−02 |
| | GGGCGG | 0.51 | 8.43E−06 | 3.45E−02 |
| | CCGGCTT | 0.07 | 2.15E−06 | 3.52E−02 |
| | CGGAAGT | 0.08 | 2.22E−06 | 3.63E−02 |
| | TGGCGGC | 0.15 | 2.52E−06 | 4.13E−02 |
| | AAGATGGC | 0.05 | 6.97E−07 | 4.57E−02 |

motif ADE was performed using the masked promoter database, consisting of regions of the promoters aligned and conserved between mouse and human. Motifs achieving a Bonferroni-corrected P-value < 0.05 are shown.

TABLE 11
Genes having an Errα binding site motif

1: NM_000065, “Homo sapiens complement component 6 (C6), mRNA”,
gi|4559405|ref|NM_000065.1|[4559405]; 2: NM_000067, “Homo sapiens carbonic anhydrase II
(CA2), mRNA”, gi|4557394|ref|NM_000067.1|[4557394]; 3: NM_000152, “Homo sapiens
glucosidase, alpha; acid (Pompe disease, glycogen storage disease”, “type II) (GAA), mRNA”,
gi|11496988|ref|NM_000152.2|[11496988]; 4: NM_000155, “Homo sapiens galactose-1-
phosphate uridylyltransferase (GALT), transcript”, “variant 1, mRNA”,
gi|22165415|ref|NM_000155.2|[22165415]; 5: NM_000164, “Homo sapiens gastric inhibitory
polypeptide receptor (GIPR), mRNA”, gi|4503998|ref|NM_000164.1|[4503998]; 6: NM_000183,
Homo sapiens hydroxyacyl-Coenzyme A dehydrogenase/3-ketoacyl-Coenzyme A,
“thiolase/enoyl-Coenzyme A hydratase (trifunctional protein), beta subunit”, “(HADHB),
mRNA”, gi|4504326|ref|NM_000183.1|[4504326]; 7: NM_000186, “Homo sapiens H factor 1
(complement) (HF1), mRNA”, gi|4504374|ref|NM_000186.1|[4504374]; 8: NM_000196,
“Homo sapiens hydroxysteroid (11-beta) dehydrogenase 2 (HSD11B2), mRNA”,
gi|31542940|ref|NM_000196.2|[31542940]; 9: NM_000219, “Homo sapiens potassium voltage-
gated channel, Isk-related family, member 1”, “(KCNE1), mRNA”,
gi|4557686|ref|NM_000219.1|[4557686]; 10: NM_000226, “Homo sapiens keratin 9
(epidermolytic palmoplantar keratoderma) (KRT9), mRNA”,
gi|4557704|ref|NM_000226.1|[4557704]; 11: NM_000236, “Homo sapiens lipase, hepatic
(LIPC), mRNA”, gi|4557722|ref|NM_000236.1|[4557722]; 12: NM_000249, “Homo sapiens
mutL homolog 1, colon cancer, nonpolyposis type 2 (E. coli) (MLH1),”, mRNA,
gi|28559089|ref|NM_000249.2|[28559089]; 13: NM_000274, “Homo sapiens ornithine
aminotransferase (gyrate atrophy) (OAT), nuclear gene”, “encoding mitochondrial protein,
mRNA”, gi|4557808|ref|NM_000274.1|[4557808]; 14: NM_000297, “Homo sapiens polycystic
kidney disease 2 (autosomal dominant) (PKD2), mRNA”,
gi|33286447|ref|NM_00297.2|[33286447]; 15: NM_000343, “Homo sapiens solute carrier
family 5 (sodium/glucose cotransporter), member 1”, “(SLC5A1), mRNA”,
gi|4507030|ref|NM_000343.1|[4507030]; 16: NM_000347, “Homo sapiens spectrin, beta,
erythrocytic (includes spherocytosis, clinical type”, “I) (SPTB), mRNA”,
gi|22507315|ref|NM_000347.3|[22507315]; 17: NM_000349, “Homo sapiens steroidogenic
acute regulatory protein (STAR), mRNA”, gi|4507250|ref|NM_000349.1|[4507250]; 18:
NM_000364, “Homo sapiens troponin T2, cardiac (TNNT2), mRNA”,
gi|4507626|ref|NM_000364.1|[4507626]; 19: NM_000372, “Homo sapiens tyrosinase
(oculocutaneous albinism IA) (TYR), mRNA”, gi|24475623|ref|NM_000372.2|[24475623]; 20:
NM_000403, “Homo sapiens galactose-4-epimerase, UDP (GALE), mRNA”,
gi|9945333|ref|NM_000403.2|[9945333]; 21: NM_000433, “Homo sapiens neutrophil cytosolic
factor 2 (65 kDa, chronic granulomatous”, “disease, autosomal 2) (NCF2), mRNA”,
gi|4557786|ref|NM_000433.1|[4557786]; 22: NM_000474, Homo sapiens twist homolog 1
(acrocephalosyndactyly 3; Saethre-Chotzen syndrome), “(Drosophila) (TWIST1), mRNA”,
gi|17978464|ref|NM_000474.2|[17978464]; 23: NM_000478, “Homo sapiens alkaline
phosphatase, liver/bone/kidney (ALPL), mRNA”, gi|13787192|ref|NM_000478.2|[13787192];
24: NM_000481,, ref|NM_000481.2|[44662837]; 25: NM_000483, “Homo sapiens
apolipoprotein C-II (APOC2), mRNA”, gi|32130517|ref|NM_000483.3|[32130517]; 26:
NM_000499, “Homo sapiens cytochrome P450, family 1, subfamily A, polypeptide 1
(CYP1A1),”, mRNA, gi|13325053|ref|NM_000499.2|[13325053]; 27: NM_000526, “Homo
sapiens keratin 14 (epidermolysis bullosa simplex, Dowling-Meara, Koebner)”, “(KRT14),
mRNA”, gi|15431309|ref|NM_000526.3|[15431309]; 28: NM_000532, “Homo sapiens
propionyl Coenzyme A carboxylase, beta polypeptide (PCCB), mRNA”,
gi|24475879|ref|NM_000532.2|[24475879]; 29: NM_000536, “Homo sapiens recombination
activating gene 2 (RAG2), mRNA”, gi|28629867|ref|NM_000536.1|[28629867]; 30:
NM_000593, “Homo sapiens transporter 1, ATP-binding cassette, sub-family B (MDR/TAP)
(TAP1),”, mRNA, gi|24797159|ref|NM_000593.4|[24797159]; 31: NM_000603, “Homo sapiens
nitric oxide synthase 3 (endothelial cell) (NOS3), mRNA”,
gi|40254421|ref|NM_000603.2|[40254421]; 32: NM_000614, “Homo sapiens ciliary
neurotrophic factor (CNTF), mRNA”, gi|25952136|ref|NM_000614.2|[25952136]; 33:
NM_000616, “Homo sapiens CD4 antigen (p55) (CD4), mRNA”,
gi|21314613|ref|NM_000616.2|[21314613]; 34: NM_000628, “Homo sapiens interleukin 10
receptor, beta (IL10RB), mRNA”, gi|24430214|ref|NM_000628.3|[24430214]; 35: NM_000634,
“Homo sapiens interleukin 8 receptor, alpha (IL8RA), mRNA”,
gi|29171679|ref|NM_000634.2|[29171679]; 36: NM_000666, “Homo sapiens aminoacylase 1
(ACY1), mRNA”, gi|4501900|ref|NM_000666.1|[4501900]; 37: NM_000688, “Homo sapiens
aminolevulinate, delta-, synthase 1 (ALAS1), transcript variant 1,”, mRNA,
gi|40316942|ref|NM_000688.4|[40316942]; 38: NM_000711,,
ref|NM_000711.1|BGLAP[4502400], This record was replaced or removed. See revision history
for details.,, 39: NM_000735, “Homo sapiens glycoprotein hormones, alpha polypeptide
(CGA), mRNA”, gi|10800407|ref|NM_000735.2|[10800407]; 40: NM_000741, “Homo sapiens
cholinergic receptor, muscarinic 4 (CHRM4), mRNA”, gi|4502820|ref|NM_000741.1|[4502820];
41: NM_000742, “Homo sapiens cholinergic receptor, nicotinic, alpha polypeptide 2
(neuronal)”, “(CHRNA2), mRNA”, gi|4502822|ref|NM_000742.1|[4502822]; 42: NM_000747,
“Homo sapiens cholinergic receptor, nicotinic, beta polypeptide 1 (muscle)”, “(CHRNB1),
mRNA”, gi|41327725|ref|NM_000747.2|[41327725]; 43: NM_000759, “Homo sapiens colony
stimulating factor 3 (granulocyte) (CSF3), transcript”, “variant 1, mRNA”,
gi|27437047|ref|NM_000759.2|[27437047]; 44: NM_000781, “Homo sapiens cytochrome P450,
family 11, subfamily A, polypeptide 1 (CYP11A1),”, “nuclear gene encoding mitochondrial
protein, mRNA”, gi|4503188|ref|NM_000781.1|[4503188]; 45: NM_000783, “Homo sapiens
cytochrome P450, family 26, subfamily A, polypeptide 1 (CYP26A1),”, “transcript variant 1,
mRNA”, gi|16933529|ref|NM_000783.2|[16933529]; 46: NM_000806, “Homo sapiens gamma-
aminobutyric acid (GABA) A receptor, alpha 1 (GABRA1), mRNA”,
gi|38327553|ref|NM_000806.3|[38327553]; 47: NM_000808, “Homo sapiens gamma-
aminobutyric acid (GABA) A receptor, alpha 3 (GABRA3), mRNA”,
gi|34734069|ref|NM_000808.2|[34734069]; 48: NM_000813, “Homo sapiens gamma-
aminobutyric acid (GABA) A receptor, beta 2 (GABRB2),”, “transcript variant 2, mRNA”,
gi|4503864|ref|NM_000813.1|[4503864]; 49: NM_000835, “Homo sapiens glutamate receptor,
ionotropic, N-methyl D-aspartate 2C (GRIN2C),”, mRNA,
gi|6006004|ref|NM_000835.2|[6006004]; 50: NM_000884, “Homo sapiens IMP (inosine
monophosphate) dehydrogenase 2 (IMPDH2), mRNA”,
gi|4504688|ref|NM_000884.1|[4504688]; 51: NM_000887, “Homo sapiens integrin, alpha X
(antigen CD11C (p150), alpha polypeptide)”, “(ITGAX), mRNA”,
gi|34452172|ref|NM_000887.3|[34452172]; 52: NM_000909, “Homo sapiens neuropeptide Y
receptor Y1 (NPY1R), mRNA”, gi|41350310|ref|NM_000909.4|[41350310]; 53: NM_000911,
“Homo sapiens opioid receptor, delta 1 (OPRD1), mRNA”,
gi|27734716|ref|NM_000911.2|[27734716]; 54: NM_000915, “Homo sapiens oxytocin, prepro-
(neurophysin I) (OXT), mRNA”, gi|12707574|ref|NM_000915.2|[12707574]; 55: NM_000916,
“Homo sapiens oxytocin receptor (OXTR), mRNA”, gi|32307151|ref|NM_000916.3|[32307151];
56: NM_000920, “Homo sapiens pyruvate carboxylase (PC), nuclear gene encoding
mitochondrial”, “protein, transcript variant A, mRNA”,
gi|11761622|ref|NM_000920.2|[11761622]; 57: NM_000928, “Homo sapiens phospholipase A2,
group IB (pancreas) (PLA2G1B), mRNA”, gi|38016927|ref|NM_000928.2|[38016927]; 58:
NM_000932, “Homo sapiens phospholipase C, beta 3 (phosphatidylinositol-specific)
(PLCB3),”, mRNA, gi|11386138|ref|NM_000932.1|[11386138]; 59: NM_000960, “Homo
sapiens prostaglandin I2 (prostacyclin) receptor (IP) (PTGIR), mRNA”,
gi|39995095|ref|NM_000960.3|[39995095]; 60: NM_001040, “Homo sapiens sex hormone-
binding globulin (SHBG), mRNA”, gi|7382459|ref|NM_001040.2|[7382459]; 61: NM_001041,
“Homo sapiens sucrase-isomaltase (SI), mRNA”, gi|4506944|ref|NM_001041.1|[4506944]; 62:
NM_001087, “Homo sapiens angio-associated, migratory cell protein (AAMP), mRNA”,
gi|4557228|ref|NM_001087.1|[4557228]; 63: NM_001094, “Homo sapiens amiloride-sensitive
cation channel 1, neuronal (degenerin) (ACCN1),”, “transcript variant 2, mRNA”,
gi|34452696|ref|NM_001094.4|[34452696]; 64: NM_001099, “Homo sapiens acid phosphatase,
prostate (ACPP), mRNA”, gi|6382063|ref|NM_001099.2|[6382063]; 65: NM_001104, “Homo
sapiens actinin, alpha 3 (ACTN3), mRNA”, gi|4557240|ref|NM_001104.1|[4557240]; 66:
NM_001118, Homo sapiens adenylate cyclase activating polypeptide 1 (pituitary) receptor,
“type I (ADCYAP1R1), mRNA”, gi|34398688|ref|NM_001118.3|[34398688]; 67: NM_001152,
Homo sapiens solute carrier family 25 (mitochondrial carrier; adenine nucleotide, “translocator),
member 5 (SLC25A5), mRNA”, gi|4502098|ref|NM_001152.1|[4502098]; 68: NM_001158,
“Homo sapiens amine oxidase, copper containing 2 (retina-specific) (AOC2),”, “transcript
variant 1, mRNA”, gi|6806880|ref|NM_001158.2|[6806880]; 69: NM_001164, “Homo sapiens
amyloid beta (A4) precursor protein-binding, family B, member 1”, “(Fe65) (APBB1), transcript
variant 1, mRNA”, gi|22035552|ref|NM_001164.2|[22035552]; 70: NM_001165, “Homo
sapiens baculoviral IAP repeat-containing 3 (BIRC3), transcript variant 1,”, mRNA,
gi|33946283|ref|NM_001165.3|[33946283]; 71: NM_001188, “Homo sapiens BCL2-
antagonist/killer 1 (BAK1), mRNA”, gi|33457353|ref|NM_001188.2|[33457353]; 72:
NM_001215, “Homo sapiens carbonic anhydrase VI (CA6), mRNA”,
gi|4557396|ref|NM_001215.1|[4557396]; 73: NM_001257, “Homo sapiens cadherin 13, H-
cadherin (heart) (CDH13), mRNA”, gi|16507956|ref|NM_001257.2|[16507956]; 74:
NM_001261, “Homo sapiens cyclin-dependent kinase 9 (CDC2-related kinase) (CDK9),
mRNA”, gi|17017983|ref|NM_001261.2|[17017983]; 75: NM_001346, “Homo sapiens
diacylglycerol kinase, gamma 90 kDa (DGKG), mRNA”,
gi|4503314|ref|NM_001346.1|[4503314]; 76: NM_001405, “Homo sapiens ephrin-A2 (EFNA2),
mRNA”, gi|27894380|ref|NM_001405.2|[27894380]; 77: NM_001425, “Homo sapiens
epithelial membrane protein 3 (EMP3), mRNA”, gi|4503562|ref|NM_001425.1|[4503562]; 78:
NM_001501, “Homo sapiens gonadotropin-releasing hormone 2 (GNRH2), transcript variant
1,”, mRNA, gi|4504056|ref|NM_001501.1|[4504056]; 79: NM_001507, “Homo sapiens G
protein-coupled receptor 38 (GPR38), mRNA”, gi|4504094|ref|NM_001507.1|[4504094]; 80:
NM_001525, “Homo sapiens hypocretin (orexin) receptor 1 (HCRTR1), mRNA”,
gi|4557636|ref|NM_001525.1|[4557636]; 81: NM_001542, “Homo sapiens immunoglobulin
superfamily, member 3 (IGSF3), mRNA”, gi|4504626|ref|NM_001542.1|[4504626]; 82:
NM_001662, “Homo sapiens ADP-ribosylation factor 5 (ARF5), mRNA”,
gi|6995999|ref|NM_001662.2|[6995999]; 83: NM_001665, “Homo sapiens ras homolog gene
family, member G (rho G) (ARHG), mRNA”, gi|4502218|ref|NM_001665.1|[4502218]; 84:
NM_001666, “Homo sapiens Rho GTPase activating protein 4 (ARHGAP4), mRNA”,
gi|41327157|ref|NM_001666.2|[41327157]; 85: NM_001702, “Homo sapiens brain-specific
angiogenesis inhibitor 1 (BAI1), mRNA”, gi|4502354|ref|NM_001702.1|[4502354]; 86:
NM_001722, “Homo sapiens polymerase (RNA) III (DNA directed) polypeptide D, 44 kDa
(POLR3D),”, mRNA, gi|4502436|ref|NM_001722.1|[4502436]; 87: NM_001766, “Homo
sapiens CD1D antigen, d polypeptide (CD1D), mRNA”,
gi|34419629|ref|NM_001766.2|[34419629]; 88: NM_001795, “Homo sapiens cadherin 5, type 2,
VE-cadherin (vascular epithelium) (CDH5), mRNA”,
gi|14589894|ref|NM_001795.2|[14589894]; 89: NM_001805, “Homo sapiens CCAAT/enhancer
binding protein (C/EBP), epsilon (CEBPE), mRNA”,
gi|28872799|ref|NM_001805.2|[28872799]; 90: NM_001807, “Homo sapiens carboxyl ester
lipase (bile salt-stimulated lipase) (CEL), mRNA”, gi|27894374|ref|NM_001807.2|[27894374];
91: NM_001823, “Homo sapiens creatine kinase, brain (CKB), mRNA”,
gi|34335231|ref|NM_001823.3|[34335231]; 92: NM_001859, “Homo sapiens solute carrier
family 31 (copper transporters), member 1 (SLC31A1),”, mRNA,
gi|40254457|ref|NM_001859.2|[40254457]; 93: NM_001864, “Homo sapiens cytochrome c
oxidase subunit VIIa polypeptide 1 (muscle) (COX7A1),”, mRNA,
gi|18105034|ref|NM_001864.2|[18105034]; 94: NM_001887, “Homo sapiens crystallin, beta B1
(CRYBB1), mRNA”, gi|21536279|ref|NM_001887.3|[21536279]; 95: NM_001888, “Homo
sapiens crystallin, mu (CRYM), mRNA”, gi|4503064|ref|NM_001888.1|[4503064]; 96:
NM_001893, “Homo sapiens casein kinase 1, delta (CSNK1D), transcript variant 1, mRNA”,
gi|20544143|ref|NM_001893.3|[20544143]; 97: NM_001895, “Homo sapiens casein kinase 2,
alpha 1 polypeptide (CSNK2A1), transcript variant”, “2, mRNA”,
gi|29570794|ref|NM_001895.2|[29570794]; 98: NM_001923, “Homo sapiens damage-specific
DNA binding protein 1, 127 kDa (DDB1), mRNA”, gi|13435358|ref|NM_001923.2|[13435358];
99: NM_001958, “Homo sapiens eukaryotic translation elongation factor 1 alpha 2 (EEF1A2),
mRNA”, gi|25453470|ref|NM_001958.2|[25453470]; 100: NM_001982, Homo sapiens v-erb-b2
erythroblastic leukemia viral oncogene homolog 3 (avian), “(ERBB3), mRNA”,
gi|4503596|ref|NM_001982.1|[4503596]; 101: NM_001998, “Homo sapiens fibulin 2 (FBLN2),
mRNA”, gi|4503664|ref|NM_001998.1|[4503664]; 102: NM_002010, “Homo sapiens fibroblast
growth factor 9 (glia-activating factor) (FGF9), mRNA”,
gi|4503706|ref|NM_002010.1|[4503706]; 103: NM_002012, “Homo sapiens fragile histidine
triad gene (FHIT), mRNA”, gi|4503718|ref|NM_002012.1|[4503718]; 104: NM_002036,,
ref|NM_002036.2|[42822886]; 105: NM_002054, “Homo sapiens glucagon (GCG), mRNA”,
gi|20302161|ref|NM_002054.2|[20302161]; 106: NM_002073, “Homo sapiens guanine
nucleotide binding protein (G protein), alpha z polypeptide”, “(GNAZ), mRNA”,
gi|4504050|ref|NM_002073.1|[4504050]; 107: NM_002083, “Homo sapiens glutathione
peroxidase 2 (gastrointestinal) (GPX2), mRNA”, gi|32967606|ref|NM_002083.2|[32967606];
108: NM_002139, “Homo sapiens RNA binding motif protein, X-linked (RBMX), mRNA”,
gi|4504450|ref|NM_002139.1|[4504450]; 109: NM_002151, “Homo sapiens hepsin
(transmembrane protease, serine 1) (HPN), transcript variant”, “2, mRNA”,
gi|4504480|ref|NM_002151.1|[4504480]; 110: NM_002157, “Homo sapiens heat shock 10 kDa
protein 1 (chaperonin 10) (HSPE1), mRNA”, gi|4504522|ref|NM_002157.1|[4504522]; 111:
NM_002193, “Homo sapiens inhibin, beta B (activin AB beta polypeptide) (INHBB), mRNA”,
gi|9257224|ref|NM_002193.1|[9257224]; 112: NM_002208, “Homo sapiens integrin, alpha E
(antigen CD103, human mucosal lymphocyte antigen”, “1; alpha polypeptide) (ITGAE),
mRNA”, gi|6007850|ref|NM_002208.3|[6007850]; 113: NM_002217, “Homo sapiens pre-alpha
(globulin) inhibitor, H3 polypeptide (ITIH3), mRNA”,
gi|10092578|ref|NM_002217.1|[10092578]; 114: NM_002220, “Homo sapiens inositol 1,4,5-
trisphosphate 3-kinase A (ITPKA), mRNA”, gi|4504788|ref|NM_002220.1|[4504788]; 115:
NM_002236, “Homo sapiens potassium voltage-gated channel, subfamily F, member 1
(KCNF1),”, mRNA, gi|27436998|ref|NM_002236.4|[27436998]; 116: NM_002238, “Homo
sapiens potassium voltage-gated channel, subfamily H (eag-related), member”, “1 (KCNH1),
transcript variant 2, mRNA”, gi|27436999|ref|NM_002238.2|[27436999]; 117: NM_002246,
“Homo sapiens potassium channel, subfamily K, member 3 (KCNK3), mRNA”,
gi|4504848|ref|NM_002246.1|[4504848]; 118: NM_002257, “Homo sapiens kallikrein 1,
renal/pancreas/salivary (KLK1), mRNA”, gi|22027643|ref|NM_002257.2|[22027643]; 119:
NM_002274, “Homo sapiens keratin 13 (KRT13), transcript variant 2, mRNA”,
gi|24234693|ref|NM_002274.2|[24234693]; 120: NM_002279, “Homo sapiens keratin, hair,
acidic, 3B (KRTHA3B), mRNA”, gi|15022816|ref|NM_002279.3|[15022816]; 121: NM_002280,
“Homo sapiens keratin, hair, acidic, 5 (KRTHA5), mRNA”,
gi|15431313|ref|NM_002280.3|[15431313]; 122: NM_002343, “Homo sapiens lactotransferrin
(LTF), mRNA”, gi|4505042|ref|NM_002343.1|[4505042]; 123: NM_002374, “Homo sapiens
microtubule-associated protein 2 (MAP2), transcript variant 1, mRNA”,
gi|14195623|ref|NM_002374.2|[14195623]; 124: NM_002378, “Homo sapiens megakaryocyte-
associated tyrosine kinase (MATK), transcript variant”, “2, mRNA”,
gi|21450841|ref|NM_002378.2|[21450841]; 125: NM_002380, “Homo sapiens matrilin 2
(MATN2), transcript variant 1, mRNA”, gi|13518036|ref|NM_002380.2|[13518036]; 126:
NM_002418, “Homo sapiens motilin (MLN), mRNA”,
gi|4557033|ref|NM_002418.1|[4557033]; 127: NM_002419, “Homo sapiens mitogen-activated
protein kinase kinase kinase 11 (MAP3K11), mRNA”,
gi|21735553|ref|NM_002419.2|[21735553]; 128: NM_002437, “Homo sapiens MpV17
transgene, murine homolog, glomerulosclerosis (MPV17), mRNA”,
gi|37059781|ref|NM_002437.3|[37059781]; 129: NM_002469, “Homo sapiens myogenic factor
6 (herculin) (MYF6), mRNA”, gi|4505298|ref|NM_002469.1|[4505298]; 130: NM_002479,
“Homo sapiens myogenin (myogenic factor 4) (MYOG), mRNA”,
gi|18765726|ref|NM_002479.2|[18765726]; 131: NM_002492, “Homo sapiens NADH
dehydrogenase (ubiquinone) 1 beta subcomplex, 5, 16 kDa”, “(NDUFB5), nuclear gene encoding
mitochondrial protein, mRNA”, gi|33519467|ref|NM_002492.2|[33519467]; 132: NM_002506,
“Homo sapiens nerve growth factor, beta polypeptide (NGFB), mRNA”,
gi|4505390|ref|NM_002506.1|[4505390]; 133: NM_002527, “Homo sapiens neurotrophin 3
(NTF3), mRNA”, gi|9845503|ref|NM_002527.2|[9845503]; 134: NM_002558, “Homo sapiens
purinergic receptor P2X, ligand-gated ion channel, 1 (P2RX1), mRNA”,
gi|27894283|ref|NM_002558.2|[27894283]; 135: NM_002590, “Homo sapiens protocadherin 8
(PCDH8), transcript variant 1, mRNA”, gi|6631101|ref|NM_002590.2|[6631101]; 136:
NM_002599, “Homo sapiens phosphodiesterase 2A, cGMP-stimulated (PDE2A), mRNA”,
gi|4505656|ref|NM_002599.1|[4505656]; 137: NM_002621, “Homo sapiens properdin P factor,
complement (PFC), mRNA”, gi|4505736|ref|NM_002621.1|[4505736]; 138: NM_002630,
“Homo sapiens progastricsin (pepsinogen C) (PGC), mRNA”,
gi|4505756|ref|NM_002630.1|[4505756]; 139: NM_002644, “Homo sapiens polymeric
immunoglobulin receptor (PIGR), mRNA”, gi|31377805|ref|NM_002644.2|[31377805]; 140:
NM_002646, “Homo sapiens phosphoinositide-3-kinase, class 2, beta polypeptide (PIK3C2B),”,
mRNA, gi|15451925|ref|NM_002646.2|[15451925]; 141: NM_002788, “Homo sapiens
proteasome (prosome, macropain) subunit, alpha type, 3 (PSMA3),”, “transcript variant 1,
mRNA”, gi|23110937|ref|NM_002788.2|[23110937]; 142: NM_002831, “Homo sapiens protein
tyrosine phosphatase, non-receptor type 6 (PTPN6),”, “transcript variant 1, mRNA”,
gi|34328900|ref|NM_002831.3|[34328900]; 143: NM_002832, “Homo sapiens protein tyrosine
phosphatase, non-receptor type 7 (PTPN7),”, “transcript variant 1, mRNA”,
gi|18375657|ref|NM_002832.2|[18375657]; 144: NM_002894, “Homo sapiens retinoblastoma
binding protein 8 (RBBP8), transcript variant 1,”, mRNA,
gi|42718012|ref|NM_002894.2|[42718012]; 145: NM_002904, “Homo sapiens RD RNA
binding protein (RDBP), mRNA”, gi|20631983|ref|NM_002904.4|[20631983]; 146: NM_002912,
“Homo sapiens REV3-like, catalytic subunit of DNA polymerase zeta (yeast)”, “(REV3L),
mRNA”, gi|4506482|ref|NM_002912.1|[4506482]; 147: NM_002930, “Homo sapiens Ras-like
without CAAX 2 (RIT2), mRNA”, gi|4506532|ref|NM_002930.1|[4506532]; 148: NM_002938,
“Homo sapiens ring finger protein 4 (RNF4), mRNA”,
gi|34305289|ref|NM_002938.2|[34305289]; 149: NM_002965, “Homo sapiens S100 calcium
binding protein A9 (calgranulin B) (S100A9), mRNA”, gi|9845520|ref|NM_002965.2|[9845520];
150: NM_002981, “Homo sapiens chemokine (C—C motif) ligand 1 (CCL1), mRNA”,
gi|4506832|ref|NM_002981.1|[4506832]; 151: NM_003002, “Homo sapiens succinate
dehydrogenase complex, subunit D, integral membrane”, “protein (SDHD), nuclear gene
encoding mitochondrial protein, mRNA”, gi|4506864|ref|NM_003002.1|[4506864]; 152:
NM_003015, “Homo sapiens secreted frizzled-related protein 5 (SFRP5), mRNA”,
gi|8400734|ref|NM_003015.2|[8400734]; 153: NM_003021, “Homo sapiens small glutamine-
rich tetratricopeptide repeat (TPR)-containing,”, “alpha (SGTA), mRNA”,
gi|38788107|ref|NM_003021.3|[38788107]; 154: NM_003042, “Homo sapiens solute carrier
family 6 (neurotransmitter transporter, GABA),”, “member 1 (SLC6A1), mRNA”,
gi|40254466|ref|NM_003042.2|[40254466]; 155: NM_003047, “Homo sapiens solute carrier
family 9 (sodium/hydrogen exchanger), isoform 1”, “(antiporter, Na+/H+, amiloride sensitive)
(SLC9A1), mRNA”, gi|27777631|ref|NM_003047.2|[27777631]; 156: NM_003055, “Homo
sapiens solute carrier family 18 (vesicular acetylcholine), member 3”, “(SLC18A3), mRNA”,
gi|4506990|ref|NM_003055.1|[4506990]; 157: NM_003059, “Homo sapiens solute carrier
family 22 (organic cation transporter), member 4”, “(SLC22A4), mRNA”,
gi|24497489|ref|NM_003059.2|[24497489]; 158: NM_003063, “Homo sapiens sarcolipin (SLN),
mRNA”, gi|4507062|ref|NM_003063.1|[4507062]; 159: NM_003085, “Homo sapiens synuclein,
beta (SNCB), mRNA”, gi|6466453|ref|NM_003085.2|[6466453]; 160: NM_003097, “Homo
sapiens small nuclear ribonucleoprotein polypeptide N (SNRPN), transcript”, “variant 1,
mRNA”, gi|29540556|ref|NM_003097.3|[29540556]; 161: NM_003105, “Homo sapiens sortilin-
related receptor, L(DLR class) A repeats-containing”, “(SORL1), mRNA”,
gi|18379347|ref|NM_003105.3|[18379347]; 162: NM_003115, “Homo sapiens UDP-N-
acteylglucosamine pyrophosphorylase 1 (UAP1), mRNA”,
gi|34147515|ref|NM_003115.3|[34147515]; 163: NM_003159, “Homo sapiens cyclin-dependent
kinase-like 5 (CDKL5), mRNA”, gi|4507280|ref|NM_003159.1|[4507280]; 164: NM_003212,
“Homo sapiens teratocarcinoma-derived growth factor 1 (TDGF1), mRNA”,
gi|4507424|ref|NM_003212.1|[4507424]; 165: NM_003216, “Homo sapiens thyrotrophic
embryonic factor (TEF), mRNA”, gi|34486096|ref|NM_003216.2|[34486096]; 166: NM_003239,
“Homo sapiens transforming growth factor, beta 3 (TGFB3), mRNA”,
gi|4507464|ref|NM_003239.1|[4507464]; 167: NM_003240, “Homo sapiens endometrial
bleeding associated factor (left-right determination,”, “factor A; transforming growth factor beta
superfamily) (EBAF), mRNA”, gi|27436880|ref|NM_003240.2|[27436880]; 168: NM_003249,
“Homo sapiens thimet oligopeptidase 1 (THOP1), mRNA”,
gi|34222291|ref|NM_003249.3|[34222291]; 169: NM_003259, “Homo sapiens intercellular
adhesion molecule 5, telencephalin (ICAM5), mRNA”,
gi|12545403|ref|NM_003259.2|[12545403]; 170: NM_003279, “Homo sapiens troponin C2, fast
(TNNC2), mRNA”, gi|40807466|ref|NM_003279.2|[40807466]; 171: NM_003325, Homo
sapiens HIR histone cell cycle regulation defective homolog A (S., “cerevisiae) (HIRA),
mRNA”, gi|21536484|ref|NM_003325.3|[21536484]; 172: NM_003334, Homo sapiens
ubiquitin-activating enzyme E1 (A1S9T and BN75 temperature, “sensitivity complementing)
(UBE1), transcript variant 1, mRNA”, gi|23510337|ref|NM_003334.2|[23510337]; 173:
NM_003341, “Homo sapiens ubiquitin-conjugating enzyme E2E 1 (UBC4/5 homolog, yeast)”,
“(UBE2E1), transcript variant 1, mRNA”, gi|33359692|ref|NM_003341.3|[33359692]; 174:
NM_003361, “Homo sapiens uromodulin (uromucoid, Tamm-Horsfall glycoprotein) (UMOD),
mRNA”, gi|4507832|ref|NM_003361.1|[4507832]; 175: NM_003364, “Homo sapiens uridine
phosphorylase 1 (UPP1), transcript variant 1, mRNA”,
gi|31742506|ref|NM_003364.2|[31742506]; 176: NM_003374, “Homo sapiens voltage-
dependent anion channel 1 (VDAC1), mRNA”, gi|4507878|ref|NM_003374.1|[4507878]; 177:
NM_003384, “Homo sapiens vaccinia related kinase 1 (VRK1), mRNA”,
gi|4507902|ref|NM_003384.1|[4507902]; 178: NM_003418, Homo sapiens zinc finger protein 9
(a cellular retroviral nucleic acid binding, “protein) (ZNF9), mRNA”,
gi|4827070|ref|NM_003418.1|[4827070]; 179: NM_003458, “Homo sapiens bassoon
(presynaptic cytomatrix protein) (BSN), mRNA”, gi|4508018|ref|NM_003458.1|[4508018]; 180:
NM_003459, “Homo sapiens solute carrier family 30 (zinc transporter), member 3
(SLC30A3),”, mRNA, gi|34222155|ref|NM_003459.3|[34222155]; 181: NM_003485, “Homo
sapiens G protein-coupled receptor 68 (GPR68), mRNA”,
gi|40217828|ref|NM_003485.2|[40217828]; 182: NM_003490, “Homo sapiens synapsin III
(SYN3), transcript variant IIIa, mRNA”, gi|19924104|ref|NM_003490.2|[19924104]; 183:
NM_003492, “Homo sapiens chromosome X open reading frame 12 (CXorf12), mRNA”,
gi|4504738|ref|NM_003492.1|[4504738]; 184: NM_003524, “Homo sapiens histone 1, H2bh
(HIST1H2BH), mRNA”, gi|21166386|ref|NM_003524.2|[21166386]; 185: NM_003526, “Homo
sapiens histone 1, H2bc (HIST1H2BC), mRNA”, gi|21166388|ref|NM_003526.2|[21166388];
186: NM_003531, “Homo sapiens histone 1, H3c (HIST1H3C), mRNA”,
gi|21071022|ref|NM_003531.2|[21071022]; 187: NM_003549, “Homo sapiens
hyaluronoglucosaminidase 3 (HYAL3), mRNA”, gi|15208650|ref|NM_003549.2|[15208650];
188: NM_003554, “Homo sapiens olfactory receptor, family 1, subfamily E, member 2
(OR1E2), mRNA”, gi|11386152|ref|NM_003554.1|[11386152]; 189: NM_003571, “Homo
sapiens beaded filament structural protein 2, phakinin (BFSP2), mRNA”,
gi|21536442|ref|NM_003571.2|[21536442]; 190: NM_003594, “Homo sapiens transcription
termination factor, RNA polymerase II (TTF2), mRNA”,
gi|40807470|ref|NM_003594.3|[40807470]; 191: NM_003602, “Homo sapiens FK506 binding
protein 6, 36 kDa (FKBP6), mRNA”, gi|17149848|ref|NM_003602.2|[17149848]; 192:
NM_003627, “Homo sapiens solute carrier family 43, member 1 (SLC43A1), mRNA”,
gi|42476323|ref|NM_003627.4|[42476323]; 193: NM_003632, “Homo sapiens contactin
associated protein 1 (CNTNAP1), mRNA”, gi|4505462|ref|NM_003632.1|[4505462]; 194:
NM_003691, “Homo sapiens serine/threonine kinase 16 (STK16), mRNA”,
gi|4505836|ref|NM_003691.1|[4505836]; 195: NM_003860, “Homo sapiens barrier to
autointegration factor 1 (BANF1), mRNA”, gi|11038645|ref|NM_003860.2|[11038645]; 196:
NM_003897, “Homo sapiens immediate early response 3 (IER3), transcript variant short,
mRNA”, gi|16554595|ref|NM_003897.2|[16554595]; 197: NM_003915, “Homo sapiens copine
I (CPNE1), transcript variant 3, mRNA”, gi|23397694|ref|NM_003915.2|[23397694]; 198:
NM_003922, Homo sapiens hect (homologous to the E6-AP (UBE3A) carboxyl terminus)
domain and, “RCC1 (CHC1)-like domain (RLD) 1 (HERC1), mRNA”,
gi|4557025|ref|NM_003922.1|[4557025]; 199: NM_003947, “Homo sapiens huntingtin-
associated protein interacting protein (duo) (HAPIP),”, mRNA,
gi|4504334|ref|NM_003947.1|[4504334]; 200: NM_003954, “Homo sapiens mitogen-activated
protein kinase kinase kinase 14 (MAP3K14), mRNA”, gi|4505396|ref|NM_003954.1|[4505396];
201: NM_003957, “Homo sapiens serine/threonine kinase 29 (STK29), mRNA”,
gi|27501463|ref|NM_003957.1|[27501463]; 202: NM_003961, “Homo sapiens rhomboid,
veinlet-like 1 (Drosophila) (RHBDL1), mRNA”, gi|4506524|ref|NM_003961.1|[4506524]; 203:
NM_003974, “Homo sapiens docking protein 2, 56 kDa (DOK2), transcript variant 1, mRNA”,
gi|41406049|ref|NM_003974.2|[41406049]; 204: NM_004051,, ref|NM_004051.3|[44680134];
205: NM_004056, “Homo sapiens carbonic anhydrase VIII (CA8), mRNA”,
gi|22027499|ref|NM_004056.3|[22027499]; 206: NM_004062, “Homo sapiens cadherin 16,
KSP-cadherin (CDH16), mRNA”, gi|16507958|ref|NM_004062.2|[16507958]; 207: NM_004074,
“Homo sapiens cytochrome c oxidase subunit VIII (COX8), mRNA”,
gi|4758043|ref|NM_004074.1|[4758043]; 208: NM_004077, “Homo sapiens citrate synthase
(CS), nuclear gene encoding mitochondrial protein,”, “transcript variant 1, mRNA”,
gi|38327624|ref|NM_004077.2|[38327624]; 209: NM_004078, “Homo sapiens cysteine and
glycine-rich protein 1 (CSRP1), mRNA”, gi|4758085|ref|NM_004078.1|[4758085]; 210:
NM_004088, “Homo sapiens deoxynucleotidyltransferase, terminal (DNTT), mRNA”,
gi|29788761|ref|NM_004088.2|[29788761]; 211: NM_004091, “Homo sapiens E2F transcription
factor 2 (E2F2), mRNA”, gi|34485718|ref|NM_004091.2|[34485718]; 212: NM_004100,
“Homo sapiens eyes absent homolog 4 (Drosophila) (EYA4), transcript variant 1,”, mRNA,
gi|26667248|ref|NM_004100.2|[26667248]; 213: NM_004106, “Homo sapiens Fc fragment of
IgE, high affinity I, receptor for; gamma”, “polypeptide (FCER1G), mRNA”,
gi|4758343|ref|NM_004106.1|[4758343]; 214: NM_004174, “Homo sapiens solute carrier
family 9 (sodium/hydrogen exchanger), isoform 3”, “(SLC9A3), mRNA”,
gi|6806920|ref|NM_004174.1|[6806920]; 215: NM_004176, “Homo sapiens sterol regulatory
element binding transcription factor 1 (SREBF1),”, mRNA,
gi|22547194|ref|NM_004176.2|[22547194]; 216: NM_004178, “Homo sapiens TAR (HIV)
RNA binding protein 2 (TARBP2), transcript variant 3,”, mRNA,
gi|19743837|ref|NM_004178.3|[19743837]; 217: NM_004260, “Homo sapiens RecQ protein-
like 4 (RECQL4), mRNA”, gi|4759029|ref|NM_004260.1|[4759029]; 218: NM_004267, “Homo
sapiens carbohydrate (N-acetylglucosamine-6-O) sulfotransferase 2 (CHST2),”, mRNA,
gi|27369496|ref|NM_004267.2|[27369496]; 219: NM_004271, “Homo sapiens lymphocyte
antigen 86 (LY86), mRNA”, gi|4758707|ref|NM_004271.1|[4758707]; 220: NM_004294,
“Homo sapiens mitochondrial translational release factor 1 (MTRF1), nuclear gene”, “encoding
mitochondrial protein, mRNA”, gi|34577119|ref|NM_004294.2|[34577119]; 221: NM_004333,
“Homo sapiens v-raf murine sarcoma viral oncogene homolog B1 (BRAF), mRNA”,
gi|33188458|ref|NM_004333.2|[33188458]; 222: NM_004344, “Homo sapiens centrin, EF-hand
protein, 2 (CETN2), mRNA”, gi|4757901|ref|NM_004344.1|[4757901]; 223: NM_004358,
“Homo sapiens cell division cycle 25B (CDC25B), transcript variant 1, mRNA”,
gi|11641416|ref|NM_004358.2|[11641416]; 224: NM_004374, “Homo sapiens cytochrome c
oxidase subunit VIc (COX6C), mRNA”, gi|17999531|ref|NM_004374.2|[17999531]; 225:
NM_004427, “Homo sapiens polyhomeotic-like 2 (Drosophila) (PHC2), transcript variant 2,
mRNA”, gi|37595529|ref|NM_004427.2|[37595529]; 226: NM_004455, “Homo sapiens
exostoses (multiple)-like 1 (EXTL1), mRNA”, gi|4758317|ref|NM_004455.1|[4758317]; 227:
NM_004470, “Homo sapiens FK506 binding protein 2, 13 kDa (FKBP2), transcript variant 1,
mRNA”, gi|17149841|ref|NM_004470.2|[17149841]; 228: NM_004484, “Homo sapiens
glypican 3 (GPC3), mRNA”, gi|5360213|ref|NM_004484.2|[5360213]; 229: NM_004514,
“Homo sapiens interleukin enhancer binding factor 1 (ILF1), transcript variant 1,”, mRNA,
gi|31563337|ref|NM_004514.2|[31563337]; 230: NM_004528, “Homo sapiens microsomal
glutathione S-transferase 3 (MGST3), mRNA”, gi|22035640|ref|NM_004528.2|[22035640]; 231:
NM_004550, “Homo sapiens NADH dehydrogenase (ubiquinone) Fe—S protein 2, 49 kDa”,
“(NADH-coenzyme Q reductase) (NDUFS2), mRNA”,
gi|34147556|ref|NM_004550.3|[34147556]; 232: NM_004590, “Homo sapiens chemokine (C—C
motif) ligand 16 (CCL16), mRNA”, gi|22538800|ref|NM_004590.2|[22538800]; 233:
NM_004604, “Homo sapiens syntaxin 4A (placental) (STX4A), mRNA”,
gi|34147603|ref|NM_004604.3|[34147603]; 234: NM_004616, “Homo sapiens transmembrane 4
superfamily member 3 (TM4SF3), mRNA”, gi|21265107|ref|NM_004616.2|[21265107]; 235:
NM_004647, “Homo sapiens D4, zinc and double PHD fingers family 1 (DPF1), mRNA”,
gi|4758797|ref|NM_004647.1|[4758797]; 236: NM_004656, Homo sapiens BRCA1 associated
protein-1 (ubiquitin carboxy-terminal hydrolase), “(BAP1), mRNA”,
gi|19718752|ref|NM_004656.2|[19718752]; 237: NM_004672, “Homo sapiens mitogen-
activated protein kinase kinase kinase 6 (MAP3K6),”, “transcript variant 1, mRNA”,
gi|24497521|ref|NM_004672.2|[24497521]; 238: NM_004704, “Homo sapiens RNA, U3 small
nucleolar interacting protein 2 (RNU3IP2), mRNA”, gi|31543556|ref|NM_004704.2|[31543556];
239: NM_004753, “Homo sapiens dehydrogenase/reductase (SDR family) member 3 (DHRS3),
mRNA”, gi|34222303|ref|NM_004753.3|[34222303]; 240: NM_004794, “Homo sapiens
RAB33A, member RAS oncogene family (RAB33A), mRNA”,
gi|34485717|ref|NM_004794.2|[34485717]; 241: NM_004798, “Homo sapiens kinesin family
member 3B (KIF3B), mRNA”, gi|31742486|ref|NM_004798.2|[31742486]; 242: NM_004810,
“Homo sapiens GRB2-related adaptor protein 2 (GRAP2), mRNA”,
gi|19913386|ref|NM_004810.2|[19913386]; 243: NM_004840, “Homo sapiens Rac/Cdc42
guanine nucleotide exchange factor (GEF) 6 (ARHGEF6),”, mRNA,
gi|22027524|ref|NM_004840.1|[22027524]; 244: NM_004858, “Homo sapiens solute carrier
family 4, sodium bicarbonate cotransporter, member 8”, “(SLC4A8), mRNA”,
gi|4759133|ref|NM_004858.1|[4759133]; 245: NM_004861, Homo sapiens cerebroside (3′-
phosphoadenylylsulfate:galactosylceramide 3′), “sulfotransferase (CST), mRNA”,
gi|4758087|ref|NM_004861.1|[4758087]; 246: NM_004870, “Homo sapiens mannose-P-
dolichol utilization defect 1 (MPDU1), mRNA”, gi|4759109|ref|NM_004870.1|[4759109]; 247:
NM_004904, “Homo sapiens cAMP responsive element binding protein 5 (CREB5), mRNA”,
gi|4758499|ref|NM_004904.1|[4758499]; 248: NM_004913, “Homo sapiens chromosome 16
open reading frame 7 (C16orf7), mRNA”, gi|4757805|ref|NM_004913.1|[4757805]; 249:
NM_004927, “Homo sapiens mitochondrial ribosomal protein L49 (MRPL49), nuclear gene
encoding”, “mitochondrial protein, mRNA”, gi|27436906|ref|NM_004927.2|[27436906]; 250:
NM_004941, “Homo sapiens DEAH (Asp-Glu-Ala-His) box polypeptide 8 (DHX8), mRNA”,
gi|4826689|ref|NM_004941.1|[4826689]; 251: NM_004959, “Homo sapiens nuclear receptor
subfamily 5, group A, member 1 (NR5A1), mRNA”, gi|24432033|ref|NM_004959.3|[24432033];
252: NM_004964, “Homo sapiens histone deacetylase 1 (HDAC1), mRNA”,
gi|13128859|ref|NM_004964.2|[13128859]; 253: NM_004987, “Homo sapiens LIM and
senescent cell antigen-like domains 1 (LIMS1), mRNA”,
gi|13518025|ref|NM_004987.2|[13518025]; 254: NM_004994, “Homo sapiens matrix
metalloproteinase 9 (gelatinase B, 92 kDa gelatinase, 92 kDa”, “type IV collagenase) (MMP9),
mRNA”, gi|4826835|ref|NM_004994.1|[4826835]; 255: NM_004997, “Homo sapiens myosin
binding protein H (MYBPH), mRNA”, gi|4826841|ref|NM_004997.1|[4826841]; 256:
NM_005006, “Homo sapiens NADH dehydrogenase (ubiquinone) Fe—S protein 1, 75 kDa”,
“(NADH-coenzyme Q reductase) (NDUFS1), nuclear gene encoding mitochondrial”, “protein,
mRNA”, gi|33519474|ref|NM_005006.5|[33519474]; 257: NM_005023, “Homo sapiens protein
geranylgeranyltransferase type I, beta subunit (PGGT1B),”, mRNA,
gi|27597101|ref|NM_005023.2|[27597101]; 258: NM_005027, “Homo sapiens
phosphoinositide-3-kinase, regulatory subunit, polypeptide 2 (p85”, “beta) (PIK3R2), mRNA”,
gi|4826907|ref|NM_005027.1|[4826907]; 259: NM_005055, “Homo sapiens receptor-associated
protein of the synapse, 43 kD (RAPSN),”, “transcript variant 1, mRNA”,
gi|38045929|ref|NM_005055.3|[38045929]; 260: NM_005070, “Homo sapiens solute carrier
family 4, anion exchanger, member 3 (SLC4A3), mRNA”,
gi|4827015|ref|NM_005070.1|[4827015]; 261: NM_005124, “Homo sapiens nucleoporin
153 kDa (NUP153), mRNA”, gi|24430145|ref|NM_005124.2|[24430145]; 262: NM_005125,
“Homo sapiens copper chaperone for superoxide dismutase (CCS), mRNA”,
gi|4826664|ref|NM_005125.1|[4826664]; 263: NM_005154, “Homo sapiens ubiquitin specific
protease 8 (USP8), mRNA”, gi|41281375|ref|NM_005154.2|[41281375]; 264: NM_005161,
“Homo sapiens angiotensin II receptor-like 1 (AGTRL1), mRNA”,
gi|34577064|ref|NM_005161.2|[34577064]; 265: NM_005163, “Homo sapiens v-akt murine
thymoma viral oncogene homolog 1 (AKT1), mRNA”, gi|4885060|ref|NM_005163.1|[4885060];
266: NM_005165, “Homo sapiens aldolase C, fructose-bisphosphate (ALDOC), mRNA”,
gi|4885062|ref|NM_005165.1|[4885062]; 267: NM_005182, “Homo sapiens carbonic anhydrase
VII (CA7), mRNA”, gi|4885100|ref|NM_005182.1|[4885100]; 268: NM_005186, “Homo
sapiens calpain 1, (mu/I) large subunit (CAPN1), mRNA”,
gi|12408655|ref|NM_005186.2|[12408655]; 269: NM_005194, “Homo sapiens
CCAAT/enhancer binding protein (C/EBP), beta (CEBPB), mRNA”,
gi|28872795|ref|NM_005194.2|[28872795]; 270: NM_005210, “Homo sapiens crystallin,
gamma B (CRYGB), mRNA”, gi|13376999|ref|NM_005210.2|[13376999]; 271: NM_005223,
“Homo sapiens deoxyribonuclease I (DNASE1), mRNA”,
gi|21361253|ref|NM_005223.2|[21361253]; 272: NM_005260, “Homo sapiens growth
differentiation factor 9 (GDF9), mRNA”, gi|6715598|ref|NM_005260.2|[6715598]; 273:
NM_005261, “Homo sapiens GTP binding protein overexpressed in skeletal muscle (GEM),”,
“transcript variant 1, mRNA”, gi|32483372|ref|NM_005261.2|[32483372]; 274: NM_005286,
“Homo sapiens G protein-coupled receptor 8 (GPR8), mRNA”,
gi|30581163|ref|NM_005286.2|[30581163]; 275: NM_005288, “Homo sapiens G protein-
coupled receptor 12 (GPR12), mRNA”, gi|4885294|ref|NM_005288.1|[4885294]; 276:
NM_005301, “Homo sapiens G protein-coupled receptor 35 (GPR35), mRNA”,
gi|33695096|ref|NM_005301.2|[33695096]; 277: NM_005302, Homo sapiens G protein-coupled
receptor 37 (endothelin receptor type B-like), “(GPR37), mRNA”,
gi|31377788|ref|NM_005302.2|[31377788]; 278: NM_005306, “Homo sapiens G protein-
coupled receptor 43 (GPR43), mRNA”, gi|4885332|ref|NM_005306.1|[4885332]; 279:
NM_005326, “Homo sapiens hydroxyacylglutathione hydrolase (HAGH), mRNA”,
gi|38327035|ref|NM_005326.3|[38327035]; 280: NM_005335, “Homo sapiens hematopoietic
cell-specific Lyn substrate 1 (HCLS1), mRNA”, gi|37059786|ref|NM_005335.3|[37059786];
281: NM_005341, “Homo sapiens GLI-Kruppel family member HKR3 (HKR3), mRNA”,
gi|4885418|ref|NM_005341.1|[4885418]; 282: NM_005393, “Homo sapiens plexin B3
(PLXNB3), mRNA”, gi|10864080|ref|NM_005393.1|[10864080]; 283: NM_005398, “Homo
sapiens protein phosphatase 1, regulatory (inhibitor) subunit 3C (PPP1R3C),”, mRNA,
gi|42476161|ref|NM_005398.3|[42476161]; 284: NM_005410, “Homo sapiens selenoprotein P,
plasma, 1 (SEPP1), mRNA”, gi|4885590|ref|NM_005410.1|[4885590]; 285: NM_005418,
“Homo sapiens suppression of tumorigenicity 5 (ST5), transcript variant 1, mRNA”,
gi|21264611|ref|NM_005418.2|[21264611]; 286: NM_005453, “Homo sapiens zinc finger
protein 297 (ZNF297), mRNA”, gi|20070223|ref|NM_005453.3|[20070223]; 287: NM_005468,
“Homo sapiens N-acetylated alpha-linked acidic dipeptidase-like 1 (NAALADL1),”, mRNA,
gi|4885506|ref|NM_005468.1|[4885506]; 288: NM_005475, “Homo sapiens lymphocyte adaptor
protein (LNK), mRNA”, gi|4885454|ref|NM_005475.1|[4885454]; 289: NM_005485, Homo
sapiens ADP-ribosyltransferase (NAD+; poly (ADP-ribose) polymerase)-like 3, “(ADPRTL3),
mRNA”, gi|11496992|ref|NM_005485.2|[11496992]; 290: NM_005550, “Homo sapiens kinesin
family member C3 (KIFC3), mRNA”, gi|19923320|ref|NM_005550.2|[19923320]; 291:
NM_005557, Homo sapiens keratin 16 (focal non-epidermolytic palmoplantar keratoderma),
“(KRT16), mRNA”, gi|24430191|ref|NM_005557.2|[24430191]; 292: NM_005560, “Homo
sapiens laminin, alpha 5 (LAMA5), mRNA”, gi|21264601|ref|NM_005560.3|[21264601]; 293:
NM_005563, “Homo sapiens stathmin 1/oncoprotein 18 (STMN1), mRNA”,
gi|13518023|ref|NM_005563.2|[13518023]; 294: NM_005593, “Homo sapiens myogenic factor
5 (MYF5), mRNA”, gi|5031928|ref|NM_005593.1|[5031928]; 295: NM_005598, “Homo
sapiens nescient helix loop helix 1 (NHLH1), mRNA”,
gi|19923328|ref|NM_005598.2|[19923328]; 296: NM_005606, “Homo sapiens legumain
(LGMN), mRNA”, gi|21914880|ref|NM_005606.3|[21914880]; 297: NM_005626, “Homo
sapiens splicing factor, arginine/serine-rich 4 (SFRS4), mRNA”,
gi|34147660|ref|NM_005626.3|[34147660]; 298: NM_005630, “Homo sapiens solute carrier
organic anion transporter family, member 2A1”, “(SLCO2A1), mRNA”,
gi|5032094|ref|NM_005630.1|[5032094]; 299: NM_005634, “Homo sapiens SRY (sex
determining region Y)-box 3 (SOX3), mRNA”, gi|30061555|ref|NM_005634.2|[30061555]; 300:
NM_005684, “Homo sapiens G protein-coupled receptor 52 (GPR52), mRNA”,
gi|5031720|ref|NM_005684.1|[5031720]; 301: NM_005698, “Homo sapiens secretory carrier
membrane protein 3 (SCAMP3), transcript variant”, “1, mRNA”,
gi|16445418|ref|NM_005698.2|[16445418]; 302: NM_005716, Homo sapiens regulator of G-
protein signalling 19 interacting protein 1, “(RGS19IP1), transcript variant 1, mRNA”,
gi|42544147|ref|NM_005716.2|[42544147]; 303: NM_005726, “Homo sapiens Ts translation
elongation factor, mitochondrial (TSFM), mRNA”, gi|21361279|ref|NM_005726.2|[21361279];
304: NM_005727, “Homo sapiens tetraspan 1 (TSPAN-1), mRNA”,
gi|21264577|ref|NM_005727.2|[21264577]; 305: NM_005747, “Homo sapiens elastase 3A,
pancreatic (protease E) (ELA3A), mRNA”, gi|21361297|ref|NM_005747.2|[21361297]; 306:
NM_005777, “Homo sapiens RNA binding motif protein 6 (RBM6), mRNA”,
gi|5032032|ref|NM_005777.1|[5032032]; 307: NM_005822, “Homo sapiens Down syndrome
critical region gene 1-like 1 (DSCR1L1), mRNA”, gi|5032234|ref|NM_005822.1|[5032234];
308: NM_005845, “Homo sapiens ATP-binding cassette, sub-family C (CFTR/MRP), member 4
(ABCC4),”, mRNA, gi|34452699|ref|NM_005845.2|[34452699]; 309: NM_005860, “Homo
sapiens follistatin-like 3 (secreted glycoprotein) (FSTL3), mRNA”,
gi|5031700|ref|NM_005860.1|[5031700]; 310: NM_005892, “Homo sapiens formin-like 1
(FMNL1), mRNA”, gi|33356147|ref|NM_005892.3|[33356147]; 311: NM_005893, “Homo
sapiens calicin (CCIN), mRNA”, gi|17738311|ref|NM_005893.1|[17738311]; 312: NM_005909,
“Homo sapiens microtubule-associated protein 1B (MAP1B), transcript variant 1,”, mRNA,
gi|14165457|ref|NM_005909.2|[14165457]; 313: NM_005959, “Homo sapiens melatonin
receptor 1B (MTNR1B), mRNA”, gi|14141172|ref|NM_005959.2|[14141172]; 314: NM_005965,
“Homo sapiens myosin, light polypeptide kinase (MYLK), transcript variant 6, mRNA”,
gi|16950600|ref|NM_005965.2|[16950600]; 315: NM_005972, “Homo sapiens pancreatic
polypeptide receptor 1 (PPYR1), mRNA”, gi|40254824|ref|NM_005972.2|[40254824]; 316:
NM_005984, Homo sapiens solute carrier family 25 (mitochondrial carrier; citrate,
“transporter), member 1 (SLC25A1), mRNA”, gi|21389314|ref|NM_005984.1|[21389314]; 317:
NM_006017, “Homo sapiens prominin 1 (PROM1), mRNA”,
gi|5174386|ref|NM_006017.1|[5174386]; 318: NM_006019, “Homo sapiens T-cell, immune
regulator 1, ATPase, H+ transporting, lysosomal V0”, “protein a isoform 3 (TCIRG1), transcript
variant 1, mRNA”, gi|19924144|ref|NM_006019.2|[19924144]; 319: NM_006067, “Homo
sapiens neighbor of COX4 (NOC4), mRNA”, gi|34147520|ref|NM_006067.3|[34147520]; 320:
NM_006090, “Homo sapiens choline/ethanolaminephosphotransferase (CEPT1), mRNA”,
gi|21735567|ref|NM_006090.2|[21735567]; 321: NM_006091, “Homo sapiens coronin, actin
binding protein, 2B (CORO2B), mRNA”, gi|24307902|ref|NM_006091.1|[24307902]; 322:
NM_006114, Homo sapiens translocase of outer mitochondrial membrane 40 homolog (yeast),
“(TOMM40), mRNA”, gi|5174722|ref|NM_006114.1|[5174722]; 323: NM_006120, “Homo
sapiens major histocompatibility complex, class II, DM alpha (HLA-DMA),”, mRNA,
gi|18765714|ref|NM_006120.2|[18765714]; 324: NM_006157, “Homo sapiens NEL-like 1
(chicken) (NELL1), mRNA”, gi|5453763|ref|NM_006157.1|[5453763]; 325: NM_006163,
“Homo sapiens nuclear factor (erythroid-derived 2), 45 kDa (NFE2), mRNA”,
gi|5453773|ref|NM_006163.1|[5453773]; 326: NM_006170, “Homo sapiens nucleolar protein 1,
120 kDa (NOL1), mRNA”, gi|5453791|ref|NM_006170.1|[5453791]; 327: NM_006172, “Homo
sapiens natriuretic peptide precursor A (NPPA), mRNA”,
gi|23510318|ref|NM_006172.1|[23510318]; 328: NM_006174, “Homo sapiens neuropeptide Y
receptor Y5 (NPY5R), mRNA”, gi|31377784|ref|NM_006174.2|[31377784]; 329: NM_006196,
“Homo sapiens poly(rC) binding protein 1 (PCBP1), mRNA”,
gi|14141164|ref|NM_006196.2|[14141164]; 330: NM_006198, “Homo sapiens Purkinje cell
protein 4 (PCP4), mRNA”, gi|5453857|ref|NM_006198.1|[5453857]; 331: NM_006205, “Homo
sapiens phosphodiesterase 6H, cGMP-specific, cone, gamma (PDE6H), mRNA”,
gi|5453867|ref|NM_006205.1|[5453867]; 332: NM_006215, “Homo sapiens serine (or cysteine)
proteinase inhibitor, clade A (alpha-1”, “antiproteinase, antitrypsin), member 4 (SERPINA4),
mRNA”, gi|21361301|ref|NM_006215.2|[21361301]; 333: NM_006228, “Homo sapiens
prepronociceptin (PNOC), mRNA”, gi|11079650|ref|NM_006228.2|[11079650]; 334:
NM_006252, “Homo sapiens protein kinase, AMP-activated, alpha 2 catalytic subunit
(PRKAA2),”, mRNA, gi|5453965|ref|NM_006252.1|[5453965]; 335: NM_006261, “Homo
sapiens prophet of Pit1, paired-like homeodomain transcription factor”, “(PROP1), mRNA”,
gi|40254838|ref|NM_006261.2|[40254838]; 336: NM_006274, “Homo sapiens chemokine (C—C
motif) ligand 19 (CCL19), mRNA”, gi|22165424|ref|NM_006274.2|[22165424]; 337:
NM_006289, “Homo sapiens talin 1 (TLN1), mRNA”,
gi|16753232|ref|NM_006289.2|[16753232]; 338: NM_006365, “Homo sapiens transcriptional
activator of the c-fos promoter (CROC4), mRNA”, gi|5453624|ref|NM_006365.1|[5453624];
339: NM_006368, “Homo sapiens cAMP responsive element binding protein 3 (CREB3),
mRNA”, gi|38327637|ref|NM_006368.4|[38327637]; 340: NM_006399, “Homo sapiens basic
leucine zipper transcription factor, ATF-like (BATF), mRNA”,
gi|18375640|ref|NM_006399.2|[18375640]; 341: NM_006442, “Homo sapiens DR1-associated
protein 1 (negative cofactor 2 alpha) (DRAP1), mRNA”,
gi|18426972|ref|NM_006442.2|[18426972]; 342: NM_006466, “Homo sapiens polymerase
(RNA) III (DNA directed) polypeptide F, 39 kDa (POLR3F),”, mRNA,
gi|33598951|ref|NM_006466.2|[33598951]; 343: NM_006477, “Homo sapiens RAS-related on
chromosome 22 (RRP22), mRNA”, gi|42476128|ref|NM_006477.2|[42476128]; 344:
NM_006565, “Homo sapiens CCCTC-binding factor (zinc finger protein) (CTCF), mRNA”,
gi|5729789|ref|NM_006565.1|[5729789]; 345: NM_006614, Homo sapiens cell adhesion
molecule with homology to L1CAM (close homolog of L1), “(CHL1), mRNA”,
gi|27894375|ref|NM_006614.2|[27894375]; 346: NM_006637, “Homo sapiens olfactory
receptor, family 5, subfamily I, member 1 (OR5I1), mRNA”,
gi|5729959|ref|NM_006637.1|[5729959]; 347: NM_006650, “Homo sapiens complexin 2
(CPLX2), mRNA”, gi|17738306|ref|NM_006650.2|[17738306]; 348: NM_006698, “Homo
sapiens bladder cancer associated protein (BLCAP), mRNA”,
gi|5729737|ref|NM_006698.1|[5729737]; 349: NM_006703, Homo sapiens nudix (nucleoside
diphosphate linked moiety X)-type motif 3, “(NUDT3), mRNA”,
gi|37622350|ref|NM_006703.2|[37622350]; 350: NM_006747, “Homo sapiens signal-induced
proliferation-associated gene 1 (SIPA1), transcript”, “variant 2, mRNA”,
gi|24497626|ref|NM_006747.2|[24497626]; 351: NM_006764, “Homo sapiens interferon-related
developmental regulator 2 (IFRD2), mRNA”, gi|21361365|ref|NM_006764.2|[21361365]; 352:
NM_006794, “Homo sapiens G protein-coupled receptor 75 (GPR75), mRNA”,
gi|5803024|ref|NM_006794.1|[5803024]; 353: NM_006810, “Homo sapiens for protein disulfide
isomerase-related (PDIR), mRNA”, gi|5803120|ref|NM_006810.1|[5803120]; 354: NM_006813,
“Homo sapiens proline-rich nuclear receptor coactivator 1 (PNRC1), mRNA”,
gi|5802981|ref|NM_006813.1|[5802981]; 355: NM_006823, “Homo sapiens protein kinase
(cAMP-dependent, catalytic) inhibitor alpha (PKIA),”, “transcript variant 1, mRNA”,
gi|32483387|ref|NM_006823.2|[32483387]; 356: NM_006841, “Homo sapiens solute carrier
family 38, member 3 (SLC38A3), mRNA”, gi|40795668|ref|NM_006841.3|[40795668]; 357:
NM_006876, “Homo sapiens UDP-GlcNAc:betaGal beta-1,3-N-acetylglucosaminyltransferase
6”, “(B3GNT6), mRNA”, gi|5802983|ref|NM_006876.1|[5802983]; 358: NM_006917, “Homo
sapiens retinoid X receptor, gamma (RXRG), mRNA”,
gi|21361386|ref|NM_006917.2|[21361386]; 359: NM_006923, “Homo sapiens stromal cell-
derived factor 2 (SDF2), mRNA”, gi|14141194|ref|NM_006923.2|[14141194]; 360: NM_006946,
“Homo sapiens spectrin, beta, non-erythrocytic 2 (SPTBN2), mRNA”,
gi|5902121|ref|NM_006946.1|[5902121]; 361: NM_006982, “Homo sapiens cartilage paired-
class homeoprotein 1 (CART1), mRNA”, gi|5901917|ref|NM_006982.1|[5901917]; 362:
NM_006998, “Homo sapiens secretagogin, EF-hand calcium binding protein (SCGN), mRNA”,
gi|15055536|ref|NM_006998.2|[15055536]; 363: NM_007000, “Homo sapiens uroplakin 1A
(UPK1A), mRNA”, gi|21264372|ref|NM_007000.2|[21264372]; 364: NM_007022, “Homo
sapiens putative tumor suppressor 101F6 (101F6), mRNA”,
gi|31541779|ref|NM_007022.3|[31541779]; 365: NM_007023, “Homo sapiens cAMP-regulated
guanine nucleotide exchange factor II (CGEF2), mRNA”,
gi|5901913|ref|NM_007023.1|[5901913]; 366: NM_007046, “Homo sapiens elastin microfibril
interfacer 1 (EMILIN1), mRNA”, gi|5901943|ref|NM_007046.1|[5901943]; 367: NM_007076,,
ref|NM_007076.2|[42794619]; 368: NM_007112, “Homo sapiens thrombospondin 3 (THBS3),
mRNA”, gi|40317629|ref|NM_007112.3|[40317629]; 369: NM_007149, “Homo sapiens zinc
finger protein 184 (Kruppel-like) (ZNF184), mRNA”,
gi|24307934|ref|NM_007149.1|[24307934]; 370: NM_007182, “Homo sapiens Ras association
(RalGDS/AF-6) domain family 1 (RASSF1), transcript”, “variant A, mRNA”,
gi|25777678|ref|NM_007182.4|[25777678]; 371: NM_007194, “Homo sapiens CHK2
checkpoint homolog (S. pombe) (CHEK2), transcript variant 1,”, mRNA,
gi|22209010|ref|NM_007194.2|[22209010]; 372: NM_007238, “Homo sapiens peroxisomal
membrane protein 4, 24 kDa (PXMP4), transcript variant”, “1, mRNA”,
gi|34452733|ref|NM_007238.3|[34452733]; 373: NM_007272, “Homo sapiens chymotrypsin C
(caldecrin) (CTRC), mRNA”, gi|11321627|ref|NM_007272.1|[11321627]; 374: NM_007312,
“Homo sapiens hyaluronoglucosaminidase 1 (HYAL1), transcript variant 1, mRNA”,
gi|24497560|ref|NM_007312.3|[24497560]; 375: NM_007357, “Homo sapiens component of
oligomeric golgi complex 2 (COG2), mRNA”, gi|6678675|ref|NM_007357.1|[6678675]; 376:
NM_012093, “Homo sapiens adenylate kinase 5 (AK5), transcript variant 2, mRNA”,
gi|28144898|ref|NM_012093.2|[28144898]; 377: NM_012105, “Homo sapiens beta-site APP-
cleaving enzyme 2 (BACE2), transcript variant a, mRNA”,
gi|21040358|ref|NM_012105.3|[21040358]; 378: NM_012109, “Homo sapiens chromosome 19
open reading frame 4 (C19orf4), mRNA”, gi|6912273|ref|NM_012109.1|[6912273]; 379:
NM_012164, “Homo sapiens F-box and WD-40 domain protein 2 (FBXW2), mRNA”,
gi|7549806|ref|NM_012164.2|[7549806]; 380: NM_012168, “Homo sapiens F-box only protein
2 (FBXO2), mRNA”, gi|15812197|ref|NM_012168.2|[15812197]; 381: NM_012191, “Homo
sapiens putative tumor suppressor (FUS2), mRNA”, gi|6912379|ref|NM_012191.1|[6912379];
382: NM_012193, “Homo sapiens frizzled homolog 4 (Drosophila) (FZD4), mRNA”,
gi|22547160|ref|NM_012193.2|[22547160]; 383: NM_012204, “Homo sapiens general
transcription factor IIIC, polypeptide 4, 90 kDa (GTF3C4),”, mRNA,
gi|6912399|ref|NM_012204.1|[6912399]; 384: NM_012225, “Homo sapiens nucleotide binding
protein 2 (MinD homolog, E. coli) (NUBP2), mRNA”, gi|6912539|ref|NM_012225.1|[6912539];
385: NM_012236, “Homo sapiens sex comb on midleg homolog 1 (Drosophila) (SCMH1),
mRNA”, gi|6912641|ref|NM_012236.1|[6912641]; 386: NM_012285, “Homo sapiens potassium
voltage-gated channel, subfamily H (eag-related), member”, “4 (KCNH4), mRNA”,
gi|6912445|ref|NM_012285.1|[6912445]; 387: NM_012311, “Homo sapiens KIN, antigenic
determinant of recA protein homolog (mouse) (KIN),”, mRNA,
gi|40068516|ref|NM_012311.2|[40068516]; 388: NM_012409, “Homo sapiens prion protein 2
(dublet) (PRND), mRNA”, gi|34335267|ref|NM_012409.2|[34335267]; 389: NM_012430,
“Homo sapiens SEC22 vesicle trafficking protein-like 2 (S. cerevisiae) (SEC22L2),”, mRNA,
gi|14591918|ref|NM_012430.2|[14591918]; 390: NM_012459, Homo sapiens translocase of
inner mitochondrial membrane 8 homolog B (yeast), “(TIMM8B), mRNA”,
gi|6912711|ref|NM_012459.1|[6912711]; 391: NM_012460, Homo sapiens translocase of inner
mitochondrial membrane 9 homolog (yeast), “(TIMM9), mRNA”,
gi|21359892|ref|NM_012460.2|[21359892]; 392: NM_012482, “Homo sapiens zinc finger
protein 281 (ZNF281), mRNA”, gi|40255235|ref|NM_012482.3|[40255235]; 393: NM_013235,
“Homo sapiens nuclear RNase III Drosha (RNASE3L), mRNA”,
gi|21359821|ref|NM_013235.2|[21359821]; 394: NM_013246, “Homo sapiens cardiotrophin-
like cytokine (CLC), mRNA”, gi|7019350|ref|NM_013246.1|[7019350]; 395: NM_013314,
“Homo sapiens B-cell linker (BLNK), mRNA”, gi|40353774|ref|NM_013314.2|[40353774]; 396:
NM_013333, “Homo sapiens epsin 1 (EPN1), mRNA”,
gi|41350200|ref|NM_013333.2|[41350200]; 397: NM_013335, “Homo sapiens GDP-mannose
pyrophosphorylase A (GMPPA), mRNA”, gi|31881778|ref|NM_013335.2|[31881778]; 398:
NM_013343, “Homo sapiens loss of heterozygosity, 3, chromosomal region 2, gene A
(LOH3CR2A),”, mRNA, gi|7106370|ref|NM_013343.1|[7106370]; 399: NM_013387, “Homo
sapiens ubiquinol-cytochrome c reductase complex (7.2 kD) (HSPC051), mRNA”,
gi|41281884|ref|NM_013387.2|[41281884]; 400: NM_013403, “Homo sapiens striatin,
calmodulin binding protein 4 (STRN4), nRNA”, gi|7019572|ref|NM_013403.1|[7019572]; 401:
NM_013441, “Homo sapiens Down syndrome critical region gene 1-like 2 (DSCR1L2),
mRNA”, gi|38455419|ref|NM_013441.2|[38455419]; 402: NM_013450, “Homo sapiens
bromodomain adjacent to zinc finger domain, 2B (BAZ2B), mRNA”,
gi|7304922|ref|NM_013450.1|[7304922]; 403: NM_014015, “Homo sapiens dexamethasone-
induced transcript (DEXI), mRNA”, gi|33620720|ref|NM_014015.3|[33620720]; 404:
NM_014099,, ref|NM_014099.1|[7662610], This record was temporarily removed by RefSeq
staff for additional review.,, 405: NM_014123,, ref|NM_014123.1|[7662539], This record was
temporarily removed by RefSeq staff for additional review.,, 406: NM_014124,,
ref|NM_014124.1|[7662541], This record was temporarily removed by RefSeq staff for
additional review.,, 407: NM_014165, “Homo sapiens chromosome 6 open reading frame 66
(C6orf66), mRNA”, gi|7661785|ref|NM_014165.1|[7661785]; 408: NM_014222, “Homo
sapiens NADH dehydrogenase (ubiquinone) 1 alpha subcomplex, 8, 19 kDa”, “(NDUFA8),
nuclear gene encoding mitochondrial protein, mRNA”,
gi|33519464|ref|NM_014222.2|[33519464]; 409: NM_014236, “Homo sapiens
glyceronephosphate O-acyltransferase (GNPAT), mRNA”,
gi|7657133|ref|NM_014236.1|[7657133]; 410: NM_014301, “Homo sapiens nitrogen fixation
cluster-like (NIFU), mRNA”, gi|24307952|ref|NM_014301.1|[24307952]; 411: NM_014332,
“Homo sapiens small muscle protein, X-linked (SMPX), mRNA”,
gi|10047089|ref|NM_014332.1|[10047089]; 412: NM_014342, “Homo sapiens mitochondrial
carrier homolog 2 (C. elegans) (MTCH2), nuclear gene”, “encoding mitochondrial protein,
mRNA”, gi|40254847|ref|NM_014342.2|[40254847]; 413: NM_014348, “Homo sapiens
POM121 membrane glycoprotein-like 1 (rat) (POM121L1), mRNA”,
gi|7657468|ref|NM_014348.1|[7657468]; 414: NM_014393, “Homo sapiens staufen, RNA
binding protein, homolog 2 (Drosophila) (STAU2), mRNA”,
gi|7657624|ref|NM_014393.1|[7657624]; 415: NM_014433, “Homo sapiens rhabdoid tumor
deletion region gene 1 (RTDR1), mRNA”, gi|22209005|ref|NM_014433.2|[22209005]; 416:
NM_014453, “Homo sapiens putative breast adenocarcinoma marker (32 kD) (BC-2),
transcript”, “variant 1, mRNA”, gi|38372936|ref|NM_014453.2|[38372936]; 417: NM_014548,
“Homo sapiens tropomodulin 2 (neuronal) (TMOD2), mRNA”,
gi|40789262|ref|NM_014548.2|[40789262]; 418: NM_014576, “Homo sapiens apobec-1
complementation factor (ACF), transcript variant 1, mRNA”,
gi|20357571|ref|NM_014576.2|[20357571]; 419: NM_014606,, ref|NM_014606.1|[7657151],
This record was temporarily removed by RefSeq staff for additional review.,, 420: NM_014617,
“Homo sapiens crystallin, gamma A (CRYGA), mRNA”,
gi|13376998|ref|NM_014617.2|[13376998]; 421: NM_014662,, ref|NM_014662.1|[7662221],
This record was temporarily removed by RefSeq staff for additional review.,, 422: NM_014674,,
ref|NM_014674.1|[7662001], This record was temporarily removed by RefSeq staff for
additional review.,, 423: NM_014685, “Homo sapiens homocysteine-inducible, endoplasmic
reticulum stress-inducible,”, “ubiquitin-like domain member 1 (HERPUD1), mRNA”,
gi|7661869|ref|NM_014685.1|[7661869]; 424: NM_014702,, ref|NM_014702.1|[7662095], This
record was temporarily removed by RefSeq staff for additional review.,, 425: NM_014731,
“Homo sapiens ProSAPiP1 protein (ProSAPiP1), mRNA”,
gi|35493938|ref|NM_014731.2|[35493938]; 426: NM_014745, “Homo sapiens KIAA0233 gene
product (KIAA0233), mRNA”, gi|7662013|ref|NM_014745.1|[7662013]; 427: NM_014748,
“Homo sapiens sorting nexin 17 (SNX17), mRNA”, gi|23238249|ref|NM_014748.2|[23238249];
428: NM_014766, “Homo sapiens secernin 1 (SCRN1), mRNA”,
gi|28461170|ref|NM_014766.2|[28461170]; 429: NM_014786, “Homo sapiens Rho guanine
nucleotide exchange factor (GEF) 17 (ARHGEF17), mRNA”,
gi|21361457|ref|NM_014786.2|[21361457]; 430: NM_014813,, ref|NM_014813.1|[7662319],
This record was temporarily removed by RefSeq staff for additional review.,, 431: NM_014814,
“Homo sapiens proteasome regulatory particle subunit p44S10 (p44S10), mRNA”,
gi|7661913|ref|NM_014814.1|[7661913]; 432: NM_014849, “Homo sapiens synaptic vesicle
glycoprotein 2A (SV2A), mRNA”, gi|41281523|ref|NM_014849.2|[41281523]; 433:
NM_014901, “Homo sapiens ring finger protein 44 (RNF44), mRNA”,
gi|42718018|ref|NM_014901.4|[42718018]; 434: NM_014907, “Homo sapiens FERM and PDZ
domain containing 1 (FRMPD1), mRNA”, gi|7662415|ref|NM_014907.1|[7662415]; 435:
NM_014912, “Homo sapiens cytoplasmic polyadenylation element binding protein 3 (CPEB3),
mRNA”, gi|41281549|ref|NM_014912.2|[41281549]; 436: NM_014926, “Homo sapiens slit and
trk like gene 3 (SLITRK3), mRNA”, gi|40217819|ref|NM_014926.2|[40217819]; 437:
NM_014952, “Homo sapiens bromo adjacent homology domain containing 1 (BAHD1),
mRNA”, gi|41281572|ref|NM_014952.2|[41281572]; 438: NM_015084, “Homo sapiens
mitochondrial ribosomal protein S27 (MRPS27), nuclear gene encoding”, “mitochondrial
protein, mRNA”, gi|16950608|ref|NM_015084.1|[16950608]; 439: NM_015089, “Homo sapiens
p53-associated parkin-like cytoplasmic protein (PARC), mRNA”,
gi|24307990|ref|NM_015089.1|[24307990]; 440: NM_015163, “Homo sapiens tripartite motif-
containing 9 (TRIM9), transcript variant 1, mRNA”, gi|29543553|ref|NM_015163.3|[29543553];
441: NM_015229, “Homo sapiens KIAA0664 protein (KIAA0664), mRNA”,
gi|40254858|ref|NM_015229.2|[40254858]; 442: NM_015343, “Homo sapiens dullard homolog
(Xenopus laevis) (DULLARD), mRNA”, gi|34222318|ref|NM_015343.3|[34222318]; 443:
NM_015362,, ref|NM_015362.3|[44662829]; 444: NM_015372, “Homo sapiens hypothetical
protein HSN44A4A (HSN44A4A), mRNA”, gi|7661723|ref|NM_015372.1|[7661723]; 445:
NM_015480, “Homo sapiens poliovirus receptor-related 3 (PVRL3), mRNA”,
gi|11386198|ref|NM_015480.1|[11386198]; 446: NM_015623,, ref|NM_015623.2|[32306520],
This record was temporarily removed by RefSeq staff for additional review.,, 447: NM_015671,,
ref|NM_015671.2|[34147332], This record was replaced or removed. See revision history for
details.,, 448: NM_015710, “Homo sapiens glioma tumor suppressor candidate region gene 2
(GLTSCR2), mRNA”, gi|21359905|ref|NM_015710.2|[21359905]; 449: NM_015926, “Homo
sapiens putative secreted protein ZSIG11 (ZSIG11), mRNA”,
gi|34147580|ref|NM_015926.3|[34147580]; 450: NM_015957, “Homo sapiens likely ortholog of
mouse monocyte macrophage 19 (MMRP19), mRNA”, gi|7705723|ref|NM_015957.1|[7705723];
451: NM_015964, “Homo sapiens brain specific protein (CGI-38), mRNA”,
gi|7706275|ref|NM_015964.1|[7706275]; 452: NM_016004, “Homo sapiens chromosome 20
open reading frame 9 (C20orf9), mRNA”, gi|7705768|ref|NM_016004.1|[7705768]; 453:
NM_016067, “Homo sapiens mitochondrial ribosomal protein S18C (MRPS18C), nuclear
gene”, “encoding mitochondrial protein, mRNA”, gi|7705629|ref|NM_016067.1|[7705629]; 454:
NM_016082, “Homo sapiens CDK5 regulatory subunit associated protein 1 (CDK5RAP1),
transcript”, “variant 2, mRNA”, gi|28872783|ref|NM_016082.3|[28872783]; 455: NM_016090,
“Homo sapiens RNA binding motif protein 7 (RBM7), mRNA”,
gi|31543547|ref|NM_016090.2|[31543547]; 456: NM_016187, “Homo sapiens bridging
integrator 2 (BIN2), mRNA”, gi|7705295|ref|NM_016187.1|[7705295]; 457: NM_016210,
“Homo sapiens g20 protein (LOC51161), mRNA”, gi|31543080|ref|NM_016210.2|[31543080];
458: NM_016231, “Homo sapiens nemo like kinase (NLK), mRNA”,
gi|42734431|ref|NM_016231.2|[42734431]; 459: NM_016239, “Homo sapiens myosin XVA
(MYO15A), mRNA”, gi|22547228|ref|NM_016239.2|[22547228]; 460: NM_016292, “Homo
sapiens heat shock protein 75 (TRAP1), mRNA”, gi|7706484|ref|NM_016292.1|[7706484]; 461:
NM_016298, “Homo sapiens muscle disease-related protein (LOC51725), mRNA”,
gi|7706492|ref|NM_016298.1|[7706492]; 462: NM_016324, “Homo sapiens zinc finger protein
274 (ZNF274), transcript variant ZNF274b, mRNA”,
gi|19743797|ref|NM_016324.2|[19743797]; 463: NM_016331, “Homo sapiens zinc finger
protein ANC_2H01 (ANC_2H01), mRNA”, gi|7705934|ref|NM_016331.1|[7705934]; 464:
NM_016352, “Homo sapiens carboxypeptidase A4 (CPA4), mRNA”,
gi|10047105|ref|NM_016352.1|[10047105]; 465: NM_016368, “Homo sapiens myo-inositol 1-
phosphate synthase A1 (ISYNA1), mRNA”, gi|21902536|ref|NM_016368.3|[21902536]; 466:
NM_016388, “Homo sapiens T-cell receptor interacting molecule (TRIM), mRNA”,
gi|7706744|ref|NM_016388.1|[7706744]; 467: NM_016649, “Homo sapiens chromosome 20
open reading frame 6 (C20orf6), mRNA”, gi|22507381|ref|NM_016649.3|[22507381]; 468:
NM_017409, “Homo sapiens homeo box C10 (HOXC10), mRNA”,
gi|24497532|ref|NM_017409.2|[24497532]; 469: NM_017410, “Homo sapiens homeo box C13
(HOXC13), mRNA”, gi|24497535|ref|NM_017410.2|[24497535]; 470: NM_017418, “Homo
sapiens deleted in esophageal cancer 1 (DEC1), mRNA”,
gi|8393249|ref|NM_017418.1|[8393249]; 471: NM_017509, “Homo sapiens kallikrein 15
(KLK15), transcript variant 4, mRNA”, gi|20302142|ref|NM_017509.2|[20302142]; 472:
NM_017528, “Homo sapiens Williams Beuren syndrome chromosome region 22 (WBSCR22),
mRNA”, gi|23199994|ref|NM_017528.2|[23199994]; 473: NM_017534, “Homo sapiens myosin,
heavy polypeptide 2, skeletal muscle, adult (MYH2), mRNA”,
gi|42476189|ref|NM_017534.2|[42476189]; 474: NM_017582, “Homo sapiens ubiquitin-
conjugating enzyme E2Q (putative) (UBE2Q), mRNA”,
gi|38045949|ref|NM_017582.5|[38045949]; 475: NM_017704, “Homo sapiens fetal globin-
inducing factor (FGIF), mRNA”, gi|41350197|ref|NM_017704.2|[41350197]; 476: NM_017705,
“Homo sapiens membrane progestin receptor gamma (MPRG), mRNA”,
gi|31377751|ref|NM_017705.2|[31377751]; 477: NM_017738, “Homo sapiens chromosome 9
open reading frame 39 (C9orf39), mRNA”, gi|8923250|ref|NM_017738.1|[8923250]; 478:
NM_017740, “Homo sapiens zinc finger, DHHC domain containing 7 (ZDHHC7), mRNA”,
gi|8923254|ref|NM_017740.1|[8923254]; 479: NM_017745, “Homo sapiens BCL6 co-repressor
(BCOR), transcript variant 1, mRNA”, gi|21071036|ref|NM_017745.4|[21071036]; 480:
NM_017746, “Homo sapiens testis expressed gene 10 (TEX10), mRNA”,
gi|8923268|ref|NM_017746.1|[8923268]; 481: NM_017786, “Homo sapiens hypothetical
protein FLJ20366 (FLJ20366), mRNA”, gi|8923340|ref|NM_017786.1|[8923340]; 482:
NM_017793, “Homo sapiens RNase P protein subunit p25 (Rpp25), mRNA”,
gi|8923354|ref|NM_017793.1|[8923354]; 483: NM_017806, “Homo sapiens hypothetical
protein FLJ20406 (FLJ20406), mRNA”, gi|8923377|ref|NM_17806.1|[8923377]; 484:
NM_017815, “Homo sapiens chromosome 14 open reading frame 94 (C14orf94), mRNA”,
gi|8923395|ref|NM_017815.1|[8923395]; 485: NM_017847, “Homo sapiens chromosome 1
open reading frame 27 (C1orf27), mRNA”, gi|20127566|ref|NM_017847.2|[20127566]; 486:
NM_017865, “Homo sapiens hypothetical protein FLJ20531 (FLJ20531), mRNA”,
gi|21361765|ref|NM_017865.2|[21361765]; 487: NM_017893, “Homo sapiens sema domain,
immunoglobulin domain (Ig), transmembrane domain (TM)”, “and short cytoplasmic domain,
(semaphorin) 4G (SEMA4G), mRNA”, gi|28872813|ref|NM_017893.2|[28872813]; 488:
NM_017901, “Homo sapiens two pore segment channel 1 (TPCN1), mRNA”,
gi|29725621|ref|NM_017901.3|[29725621]; 489: NM_017915, “Homo sapiens hypothetical
protein FLJ20641 (FLJ20641), mRNA”, gi|8923595|ref|NM_017915.1|[8923595]; 490:
NM_017941, “Homo sapiens lung cancer-related protein 8 (HLC-8), mRNA”,
gi|34222156|ref|NM_017941.3|[34222156]; 491: NM_017961,, ref|NM_017961.3|[31982883],
This record was temporarily removed by RefSeq staff for additional review.,, 492: NM_017991,
“Homo sapiens hypothetical protein FLJ10081 (FLJ10081), mRNA”,
gi|21361733|ref|NM_017991.3|[21361733]; 493: NM_018005,, ref|NM_018005.1|[8922245],
This record was replaced or removed. See revision history for details.,, 494: NM_018019,
“Homo sapiens mediator subunit 25 (MED25), mRNA”,
gi|22907057|ref|NM_018019.2|[2290705]; 495: NM_018026, “Homo sapiens phosphofurin
acidic cluster sorting protein 1 (PACS1), mRNA”, gi|30089915|ref|NM_018026.2|[30089915];
496: NM_018058, “Homo sapiens cartilage acidic protein 1 (CRTAC1), mRNA”,
gi|42415498|ref|NM_018058.2|[42415498]; 497: NM_018125, “Homo sapiens hypothetical
protein FLJ10521 (FLJ10521), mRNA”, gi|33354274|ref|NM_018125.2|[33354274]; 498:
NM_018157, “Homo sapiens brain synembryn (hSyn), mRNA”,
gi|8922554|ref|NM_018157.1|[8922554]; 499: NM_018163, “Homo sapiens hypothetical
protein FLJ10634 (FLJ10634), mRNA”, gi|8922562|ref|NM_018163.1|[8922562]; 500:
NM_018176, “Homo sapiens leucine-rich repeat LGI family, member 2 (LGI2), mRNA”,
gi|21313637|ref|NM_018176.2|[21313637]; 501: NM_018180, “Homo sapiens DEAH (Asp-
Glu-Ala-His) box polypeptide 32 (DHX32), mRNA”,
gi|20336299|ref|NM_018180.2|[20336299]; 502: NM_018192, “Homo sapiens myxoid
liposarcoma associated protein 4 (MLAT4), mRNA”,
gi|27764881|ref|NM_018192.2|[27764881]; 503: NM_018195, “Homo sapiens hypothetical
protein FLJ10726 (FLJ10726), mRNA”, gi|40254918|ref|NM_018195.2|[40254918]; 504:
NM_018206, “Homo sapiens vacuolar protein sorting 35 (yeast) (VPS35), mRNA”,
gi|41352714|ref|NM_018206.3|[41352714]; 505: NM_018233, “Homo sapiens hypothetical
protein FLJ10826 (FLJ10826), mRNA”, gi|42476029|ref|NM_018233.2|[42476029]; 506:
NM_018245, “Homo sapiens hypothetical protein FLJ10851 (FLJ10851), mRNA”,
gi|8922715|ref|NM_018245.1|[8922715]; 507: NM_018261, “Homo sapiens SEC3-like 1 (S. cerevisiae)
(SEC3L1), transcript variant 1, mRNA”, gi|30410719|ref|NM_018261.2|[30410719];
508: NM_018303, “Homo sapiens SEC5-like 1 (S. cerevisiae) (SEC5L1), mRNA”,
gi|30581133|ref|NM_018303.41|[30581133]; 509: NM_018306, “Homo sapiens hypothetical
protein FLJ11036 (FLJ11036), mRNA”, gi|31542666|ref|NM_018306.2|[31542666]; 510:
NM_018327, “Homo sapiens chromosome 20 open reading frame 38 (C20orf38), mRNA”,
gi|8922874|ref|NM_018327.1|[8922874]; 511: NM_018330, “Homo sapiens KIAA1598 protein
(KIAA1598), mRNA”, gi|21314680|ref|NM_018330.2|[21314680]; 512: NM_018404, “Homo
sapiens centaurin, alpha 2 (CENTA2), mRNA”, gi|8923762|ref|NM_018404.1|[8923762]; 513:
NM_018430, “Homo sapiens translin-associated factor X interacting protein 1 (TSNAXIP1),
mRNA”, gi|8923845|ref|NM_018430.1|[8923845]; 514: NM_018431, “Homo sapiens docking
protein 5 (DOK5), transcript variant 1, mRNA”, gi|29544725|ref|NM_018431.2|[29544725];
515: NM_018459,, ref|NM_018459.1|[8922103], This record was replaced or removed. See
revision history for details.,, 516: NM_018465, “Homo sapiens chromosome 9 open reading
frame 46 (C9orf46), mRNA”, gi|8923931|ref|NM_018465.1|[8923931]; 517: NM_018484,
“Homo sapiens solute carrier family 22 (organic anion/cation transporter), member”, “11
(SLC22A11), mRNA”, gi|24497483|ref|NM_018484.2|[24497483]; 518: NM_018518, Homo
sapiens MCM10 minichromosome maintenance deficient 10 (S. cerevisiae), “(MCM10),
transcript variant 2, mRNA”, gi|33383234|ref|NM_018518.3|[33383234]; 519: NM_018558,
“Homo sapiens gamma-aminobutyric acid (GABA) receptor, theta (GABRQ), mRNA”,
gi|8924257|ref|NM_018558.1|[8924257]; 520: NM_018562,, ref|NM_018562.1|[8923971], This
record was temporarily removed by RefSeq staff for additional review.,, 521: NM_018584,
“Homo sapiens calcium/calmodulin-dependent protein kinase II (CaMKIINalpha), mRNA”,
gi|31324542|ref|NM_018584.4|[31324542]; 522: NM_018608,, ref|NM_018608.1|[8924095],
This record was temporarily removed by RefSeq staff for additional review.,, 523: NM_018641,
“Homo sapiens carbohydrate (chondroitin 4) sulfotransferase 12 (CHST12), mRNA”,
gi|20070291|ref|NM_018641.2|[20070291]; 524: NM_018947, “Homo sapiens cytochrome c,
somatic (CYCS), nuclear gene encoding mitochondrial”, “protein, mRNA”,
gi|34328939|ref|NM_018947.4|[34328939]; 525: NM_018957, “Homo sapiens SH3-domain
binding protein 1 (SH3BP1), mRNA”, gi|15147251|ref|NM_018957.2|[15147251]; 526:
NM_018959, “Homo sapiens DAZ associated protein 1 (DAZAP1), transcript variant 2,
mRNA”, gi|25470885|ref|NM_018959.2|[25470885]; 527: NM_018970, “Homo sapiens G
protein-coupled receptor 85 (GPR85), mRNA”, gi|31377760|ref|NM_018970.3|[31377760]; 528:
NM_018993, “Homo sapiens Ras and Rab interactor 2 (RIN2), mRNA”,
gi|35493905|ref|NM_018993.2|[35493905]; 529: NM_019028, “Homo sapiens HIP14-related
protein (HIP14L), mRNA”, gi|9506622|ref|NM_019028.1|[9506622]; 530: NM_019044, “Homo
sapiens hypothetical protein FLJ10996 (FLJ10996), mRNA”,
gi|21361622|ref|NM_019044.2|[21361622]; 531: NM_019063, “Homo sapiens echinoderm
microtubule associated protein like 4 (EML4), mRNA”,
gi|19923496|ref|NM_019063.2|[19923496]; 532: NM_019099, “Homo sapiens hypothetical
protein LOC55924 (LOC55924), transcript variant 1,”, mRNA,
gi|39545578|ref|NM_019099.3|[39545578]; 533: NM_019617, “Homo sapiens gastrokine 1
(GKN1), mRNA”, gi|27894363|ref|NM_019617.2|[27894363]; 534: NM_019618, “Homo
sapiens interleukin 1 family, member 9 (IL1F9), mRNA”,
gi|27894314|ref|NM_019618.2|[27894314]; 535: NM_020170, “Homo sapiens hypothetical
protein from EUROIMAGE 2021883 (LOC56926), mRNA”,
gi|24308184|ref|NM_020170.1|[24308184]; 536: NM_020188, “Homo sapiens DC13 protein
(DC13), mRNA”, gi|42476040|ref|NM_020188.2|[42476040]; 537: NM_020228, “Homo
sapiens PR domain containing 10 (PRDM10), transcript variant 1, mRNA”,
gi|41349457|ref|NM_020228.2|[41349457]; 538: NM_020237, “Homo sapiens chromosome 8
open reading frame 17 (C8orf17), mRNA”, gi|9910447|ref|NM_020237.1|[9910447]; 539:
NM_020346, Homo sapiens solute carrier family 17 (sodium-dependent inorganic phosphate,
“cotransporter), member 6 (SLC17A6), mRNA”, gi|9966810|ref|NM_020346.1|[9966810]; 540:
NM_020418, “Homo sapiens poly(rC) binding protein 4 (PCBP4), transcript variant 1, mRNA”,
gi|14670367|ref|NM_020418.2|[14670367]; 541: NM_020456, “Homo sapiens chromosome 13
open reading frame 1 (C13orf1), mRNA”, gi|20531764|ref|NM_020456.1|[20531764]; 542:
NM_020465, “Homo sapiens NDRG family member 4 (NDRG4), mRNA”,
gi|14165263|ref|NM_020465.1|[14165263]; 543: NM_020470, “Homo sapiens Yip1 interacting
factor homolog (S. cerevisiae) (YIF1), mRNA”, gi|9994168|ref|NM_020470.1|[9994168]; 544:
NM_020547, “Homo sapiens anti-Mullerian hormone receptor, type II (AMHR2), mRNA”,
gi|10198655|ref|NM_020547.1|[10198655]; 545: NM_020990, “Homo sapiens creatine kinase,
mitochondrial 1 (ubiquitous) (CKMT1), nuclear gene”, “encoding mitochondrial protein,
mRNA”, gi|11641403|ref|NM_020990.2|[11641403]; 546: NM_020999, “Homo sapiens
neurogenin 3 (NEUROG3), mRNA”, gi|10337610|ref|NM_020999.1|[10337610]; 547:
NM_021018, “Homo sapiens histone 1, H3f (HIST1H3F), mRNA”,
gi|21396497|ref|NM_021018.2|[21396497]; 548: NM_021025, “Homo sapiens T-cell leukemia,
homeobox 3 (TLX3), mRNA”, gi|10440563|ref|NM_021025.1|[10440563]; 549: NM_021062,
“Homo sapiens histone 1, H2bb (HIST1H2BB), mRNA”,
gi|19924303|ref|NM_021062.2|[19924303]; 550: NM_021067,, ref|NM_021067.1|[10800147],
This record was temporarily removed by RefSeq staff for additional review.,, 551: NM_021082,
“Homo sapiens solute carrier family 15 (H+/peptide transporter), member 2”, “(SLC15A2),
mRNA”, gi|31543623|ref|NM_021082.2|[31543623]; 552: NM_021161, “Homo sapiens
potassium channel, subfamily K, member 10 (KCNK10), transcript”, “variant 1, mRNA”,
gi|20143942|ref|NM_021161.3|[20143942]; 553: NM_021174, “Homo sapiens p30 DBC protein
(DBC-1), transcript variant 1, mRNA”, gi|40548406|ref|NM_021174.4|[40548406]; 554:
NM_021176, Homo sapiens islet-specific glucose-6-phosphatase catalytic subunit-related,
“protein (IGRP), mRNA”, gi|10863974|ref|NM_021176.1|[10863974]; 555: NM_021184,
“Homo sapiens chromosome 6 open reading frame 47 (C6orf47), mRNA”,
gi|10863984|ref|NM_021184.1|[10863984]; 556: NM_021198, “Homo sapiens CTD (carboxy-
terminal domain, RNA polymerase II, polypeptide A)”, “small phosphatase 1 (CTDSP1),
mRNA”, gi|10864008|ref|NM_021198.1|[10864008]; 557: NM_021249, “Homo sapiens sorting
nexin 6 (SNX6), transcript variant 1, mRNA”, gi|23111048|ref|NM_021249.2|[23111048]; 558:
NM_021259, “Homo sapiens transmembrane protein 8 (five membrane-spanning domains)
(TMEM8),”, mRNA, gi|10864068|ref|NM_021259.1|[10864068]; 559: NM_021639, “Homo
sapiens hypothetical protein SP192 (SP192), mRNA”,
gi|40255032|ref|NM_021639.3|[40255032]; 560: NM_021812, “Homo sapiens
blepharophimosis, epicanthus inversus and ptosis, candidate 1”, “(BPESC1), mRNA”,
gi|11141882|ref|NM_021812.1|[11141882]; 561: NM_021815, “Homo sapiens solute carrier
family 5 (choline transporter), member 7 (SLC5A7),”, mRNA,
gi|21361898|ref|NM_021815.2|[21361898]; 562: NM_021819, “Homo sapiens lectin, mannose-
binding, 1 like (LMAN1L), mRNA”, gi|11141890|ref|NM_021819.1|[11141890]; 563:
NM_021830, “Homo sapiens progressive external ophthalmoplegia 1 (PEO1), mRNA”,
gi|39725941|ref|NM_021830.3|[39725941]; 564: NM_021833, “Homo sapiens uncoupling
protein 1 (mitochondrial, proton carrier) (UCP1),”, “nuclear gene encoding mitochondrial
protein, mRNA”, gi|21614550|ref|NM_021833.3|[21614550]; 565: NM_021926, “Homo sapiens
aristaless-like homeobox 4 (ALX4), mRNA”, gi|11496266|ref|NM_021926.1|[11496266]; 566:
NM_021934, “Homo sapiens hypothetical protein FLJ11773 (FLJ11773), mRNA”,
gi|34222337|ref|NM_021934.3|[34222337]; 567: NM_021969, “Homo sapiens nuclear receptor
subfamily 0, group B, member 2 (NR0B2), mRNA”, gi|13259502|ref|NM_021969.1|[13259502];
568: NM_021981,, ref|NM_021981.1|[11415055], This record was temporarily removed by
RefSeq staff for additional review.,, 569: NM_022039, “Homo sapiens split hand/foot
malformation (ectrodactyly) type 3 (SHFM3), mRNA”,
gi|24475655|ref|NM_022039.2|[24475655]; 570: NM_022054, “Homo sapiens potassium
channel, subfamily K, member 13 (KCNK13), mRNA”,
gi|16306554|ref|NM_022054.2|[16306554]; 571: NM_022064, “Homo sapiens ring finger
protein 123 (RNF123), mRNA”, gi|37588868|ref|NM_022064.2|[37588868]; 572: NM_022082,
“Homo sapiens chromosome 20 open reading frame 59 (C20orf59), mRNA”,
gi|31542262|ref|NM_022082.2|[31542262]; 573: NM_022114, “Homo sapiens PR domain
containing 16 (PRDM16), transcript variant 1, mRNA”,
gi|41349469|ref|NM_022114.2|[41349469]; 574: NM_022120, “Homo sapiens 3-oxoacid CoA
transferase 2 (OXCT2), mRNA”, gi|11545840|ref|NM_022120.1|[11545840]; 575: NM_022131,
“Homo sapiens calsyntenin 2 (CLSTN2), mRNA”, gi|11545860|ref|NM_022131.1|[11545860];
576: NM_022135, “Homo sapiens popeye domain containing 2 (POPDC2), mRNA”,
gi|22209003|ref|NM_022135.2|[22209003]; 577: NM_022168, “Homo sapiens melanoma
differentiation associated protein-5 (MDA5), mRNA”,
gi|27886567|ref|NM_022168.2|[27886567]; 578: NM_022354, “Homo sapiens spermatogenesis
associated 1 (SPATA1), mRNA”, gi|11641266|ref|NM_022354.1|[11641266]; 579: NM_022449,
“Homo sapiens RAB17, member RAS oncogene family (RAB17), mRNA”,
gi|11967980|ref|NM_022449.1|[11967980]; 580: NM_022452, “Homo sapiens fibrosin 1
(FBS1), mRNA”, gi|11967986|ref|NM_022452.1|[11967986]; 581: NM_022489, “Homo sapiens
hypothetical protein FLJ22056 (FLJ22056), mRNA”,
gi|11968044|ref|NM_022489.1|[11968044]; 582: NM_022494, “Homo sapiens zinc finger,
DHHC domain containing 6 (ZDHHC6), mRNA”, gi|11968052|ref|NM_022494.1|[11968052];
583: NM_022568, “Homo sapiens aldehyde dehydrogenase 8 family, member A1 (ALDH8A1),
transcript”, “variant 1, mRNA”, gi|25952149|ref|NM_022568.2|[25952149]; 584: NM_022569,
“Homo sapiens N-deacetylase/N-sulfotransferase (heparan glucosaminyl) 4 (NDST4),”, mRNA,
gi|12007649|ref|NM_022569.1|[12007649]; 585: NM_022727, “Homo sapiens HpaII tiny
fragments locus 9C (HTF9C), transcript variant 2, mRNA”,
gi|21361611|ref|NM_022727.3|[21361611]; 586: NM_022748, “Homo sapiens tensin-like SH2
domain-containing 1 (TENS1), mRNA”, gi|17511208|ref|NM_022748.6|[17511208]; 587:
NM_022751, “Homo sapiens chromosome 18 open reading frame 11 (C18orf11), mRNA”,
gi|12232414|ref|NM_022751.1|[12232414]; 588: NM_022754, “Homo sapiens sideroflexin 1
(SFXN1), mRNA”, gi|40255158|ref|NM_022754.4|[40255158]; 589: NM_022765, Homo
sapiens NEDD9 interacting protein with calponin homology and LIM domains, “(NICAL),
mRNA”, gi|20127615|ref|NM_022765.2|[20127615]; 590: NM_022766, “Homo sapiens
ceramide kinase (CERK), transcript variant 1, mRNA”,
gi|32967301|ref|NM_022766.4|[32967301]; 591: NM_022771, “Homo sapiens TBC1 domain
family, member 15 (TBC1D15), mRNA”, gi|37059748|ref|NM_022771.3|[37059748]; 592:
NM_022779, “Homo sapiens DEAD (Asp-Glu-Ala-Asp) box polypeptide 31 (DDX31),
transcript”, “variant 1, mRNA”, gi|20336296|ref|NM_022779.7|[20336296]; 593: NM_023009,
“Homo sapiens MARCKS-like protein (MLP), mRNA”,
gi|32401423|ref|NM_023009.4|[32401423]; 594: NM_023112, “Homo sapiens chromosome 14
open reading frame 137 (C14orf137), mRNA”, gi|31881722|ref|NM_023112.2|[31881722]; 595:
NM_023933, “Homo sapiens hypothetical protein MGC2494 (MGC2494), mRNA”,
gi|13027599|ref|NM_023933.1|[13027599]; 596: NM_024034, Homo sapiens ganglioside-
induced differentiation-associated protein 1-like 1, “(GDAP1L1), mRNA”,
gi|30581159|ref|NM_024034.3|[30581159]; 597: NM_024057, “Homo sapiens nucleoporin
Nup37 (Nup37), mRNA”, gi|34222120|ref|NM_024057.2|[34222120]; 598: NM_024294,
“Homo sapiens hypothetical protein MGC4614 (MGC4614), mRNA”,
gi|13236513|ref|NM_024294.1|[13236513]; 599: NM_024323, “Homo sapiens hypothetical
protein MGC11271 (MGC11271), mRNA”, gi|31543147|ref|NM_024323.3|[31543147]; 600:
NM_024334, “Homo sapiens hypothetical protein MGC3222 (MGC3222), mRNA”,
gi|13236586|ref|NM_024334.1|[13236586]; 601: NM_024493, “Homo sapiens zinc finger
protein 306 (ZNF306), mRNA”, gi|24308296|ref|NM_024493.1|[24308296]; 602: NM_024506,
“Homo sapiens galactosidase, beta 1-like (GLB1L), mRNA”,
gi|40255042|ref|NM_024506.3|[40255042]; 603: NM_024515, “Homo sapiens hypothetical
protein MGC4645 (MGC4645), mRNA”, gi|34147381|ref|NM_024515.2|[34147381]; 604:
NM_024523, “Homo sapiens GRIP and coiled-coil domain-containing 1 (GCC1), mRNA”,
gi|34305454|ref|NM_024523.5|[34305454]; 605: NM_024546, “Homo sapiens chromosome 13
open reading frame 7 (C13orf7), mRNA”, gi|21362045|ref|NM_024546.2|[21362045]; 606:
NM_024560, “Homo sapiens FLJ21963 protein (FLJ21963), mRNA”,
gi|38505216|ref|NM_024560.2|[38505216]; 607: NM_024589, “Homo sapiens leucine zipper
domain protein (FLJ22386), mRNA”, gi|13375778|ref|NM_024589.1|[13375778]; 608:
NM_024604, “Homo sapiens hypothetical protein FLJ21908 (FLJ21908), mRNA”,
gi|13375808|ref|NM_024604.1|[13375808]; 609: NM_024624, Homo sapiens SMC6 structural
maintenance of chromosomes 6-like 1 (yeast), “(SMC6L1), mRNA”,
gi|31543646|ref|NM_024624.2|[31543646]; 610: NM_024626, “Homo sapiens immune
costimulatory protein B7-H4 (B7-H4), mRNA”, gi|13375849|ref|NM_024626.1|[13375849];
611: NM_024630, “Homo sapiens zinc finger, DHHC domain containing 14 (ZDHHC14),
mRNA”, gi|24371240|ref|NM_024630.2|[24371240]; 612: NM_024643, “Homo sapiens
chromosome 14 open reading frame 140 (C14orf140), mRNA”,
gi|13375882|ref|NM_024643.1|[13375882]; 613: NM_024671, “Homo sapiens hypothetical
protein FLJ23436 (FLJ23436), mRNA”, gi|20127628|ref|NM_024671.2|[20127628]; 614:
NM_024696, “Homo sapiens hypothetical protein FLJ23058 (FLJ23058), mRNA”,
gi|13375978|ref|NM_024696.1|[13375978]; 615: NM_024713, “Homo sapiens hypothetical
protein FLJ22557 (FLJ22557), mRNA”, gi|13376012|ref|NM_024713.1|[13376012]; 616:
NM_024728, “Homo sapiens chromosome 7 open reading frame 10 (C7orf10), mRNA”,
gi|13376041|ref|NM_024728.1|[13376041]; 617: NM_024731, “Homo sapiens chromosome 16
open reading frame 44 (C16orf44), mRNA”, gi|31542245|ref|NM_024731.2|[31542245]; 618:
NM_024734, “Homo sapiens calmin (calponin-like, transmembrane) (CLMN), mRNA”,
gi|19923598|ref|NM_024734.2|[19923598]; 619: NM_024754, “Homo sapiens hypothetical
protein FLJ12598 (FLJ12598), mRNA”, gi|20127633|ref|NM_024754.2|[20127633]; 620:
NM_024778, “Homo sapiens ring finger protein 127 (RNF127), mRNA”,
gi|37622895|ref|NM_024778.3|[37622895]; 621: NM_024783, “Homo sapiens hypothetical
protein FLJ23598 (FLJ23598), mRNA”, gi|31657118|ref|NM_024783.2|[31657118]; 622:
NM_024799, “Homo sapiens hypothetical protein FLJ13224 (FLJ13224), mRNA”,
gi|13376172|ref|NM_024799.1|[13376172]; 623: NM_024807, “Homo sapiens chromosome 6
open reading frame 76 (C6orf76), mRNA”, gi|13376188|ref|NM_024807.1|[13376188]; 624:
NM_024820, “Homo sapiens KIAA1608 (KIAA1608), mRNA”,
gi|13449264|ref|NM_024820.1|[13449264]; 625: NM_024827, “Homo sapiens histone
deacetylase 11 (HDAC11), mRNA”, gi|13376227|ref|NM_024827.1|[13376227]; 626:
NM_024874, “Homo sapiens polycystic kidney disease 1-like (PKD1-like), transcript variant
1,”, mRNA, gi|33359220|ref|NM_024874.3|[33359220]; 627: NM_024882, “Homo sapiens
chromosome 6 open reading frame 155 (C6orf155), mRNA”,
gi|13376326|ref|NM_024882.1|[13376326]; 628: NM_024912,, ref|NM_024912.1|[13376375],
This record was temporarily removed by RefSeq staff for additional review.,, 629: NM_024958,
“Homo sapiens chromosome 20 open reading frame 98 (C20orf98), mRNA”,
gi|13376446|ref|NM_024958.1|[13376446]; 630: NM_024969, “Homo sapiens TGF-beta
induced apotosis protein 2 (TAIP-2), mRNA”, gi|23346411|ref|NM_024969.2|[23346411]; 631:
NM_025026, “Homo sapiens hypothetical protein FLJ14107 (FLJ14107), mRNA”,
gi|13376547|ref|NM_025026.1|[13376547]; 632: NM_025079, “Homo sapiens hypothetical
protein FLJ23231 (FLJ23231), mRNA”, gi|13376631|ref|NM_025079.1|[13376631]; 633:
NM_025093,, ref|NM_025093.1|[13376653], This record was temporarily removed by RefSeq
staff for additional review.,, 634: NM_025100, “Homo sapiens chromosome 14 open reading
frame 157 (C14orf157), mRNA”, gi|13376666|ref|NM_025100.1|[13376666]; 635: NM_025137,
“Homo sapiens hypothetical protein FLJ21439 (FLJ21439), mRNA”,
gi|33636747|ref|NM_025137.2|[33636747]; 636: NM_025140, “Homo sapiens limkain beta 2
(FLJ22471), mRNA”, gi|13376724|ref|NM_025140.1|[13376724]; 637: NM_025152, “Homo
sapiens chromosome 14 open reading frame 127 (C14orf127), mRNA”,
gi|13376746|ref|NM_025152.1|[13376746]; 638: NM_025212, “Homo sapiens CXXC finger 4
(CXXC4), mRNA”, gi|13376815|ref|NM_025212.1|[13376815]; 639: NM_025236, “Homo
sapiens ring finger protein 39 (RNF39), transcript variant 1, mRNA”,
gi|25777714|ref|NM_025236.2|[25777714]; 640: NM_030769, Homo sapiens N-
acetylneuraminate pyruvate lyase (dihydrodipicolinate synthase), “(NPL), mRNA”,
gi|13540532|ref|NM_030769.1|[13540532]; 641: NM_030785, “Homo sapiens radial
spokehead-like 1 (RSHL1), mRNA”, gi|13540558|ref|NM_030785.1|[13540558]; 642:
NM_030786, “Homo sapiens intermediate filament protein syncoilin (SYNCOILIN), mRNA”,
gi|13540560|ref|NM_030786.1|[13540560]; 643: NM_030804,, ref|NM_030804.1|[13540591],
This record was temporarily removed by RefSeq staff for additional review.,, 644: NM_030818,
“Homo sapiens hypothetical protein MGC10471 (MGC10471), mRNA”,
gi|34147391|ref|NM_030818.2|[34147391]; 645: NM_030903, “Homo sapiens olfactory
receptor, family 2, subfamily W, member 1 (OR2W1), mRNA”,
gi|13624328|ref|NM_030903.1|[13624328]; 646: NM_030981, “Homo sapiens RAB1B, member
RAS oncogene family (RAB1B), mRNA”, gi|13569961|ref|NM_030981.1|[13569961]; 647:
NM_031219, “Homo sapiens hypothetical protein MGC12904 (MGC12904), mRNA”,
gi|31377665|ref|NM_031219.2|[31377665]; 648: NM_031269,, ref|NM_031269.1|[13775169],
This record was temporarily removed by RefSeq staff for additional review.,, 649: NM_031284,
“Homo sapiens ATP-dependent glucokinase (ADP-GK), mRNA”,
gi|31542508|ref|NM_031284.3|[31542508]; 650: NM_031294, “Homo sapiens hypothetical
protein DKFZp586M1120 (DKFZP586M1120), mRNA”,
gi|33636688|ref|NM_031294.2|[33636688]; 651: NM_031298, “Homo sapiens hypothetical
protein MGC2963 (MGC2963), mRNA”, gi|13775219|ref|NM_031298.1|[13775219]; 652:
NM_031450, “Homo sapiens hypothetical protein p5326 (P5326), mRNA”,
gi|31543378|ref|NM_031450.2[31543378]; 653: NM_032042, “Homo sapiens hypothetical
protein DKFZp564D172 (DKFZP564D172), mRNA”,
gi|37059749|ref|NM_032042.3|[37059749]; 654: NM_032179, “Homo sapiens hypothetical
protein FLJ20542 (FLJ20542), mRNA”, gi|14149862|ref|NM_032179.1|[14149862]; 655:
NM_032204, “Homo sapiens ASC-1 complex subunit P100 (ASC1p100), mRNA”,
gi|34147616|ref|NM_032204.3|[34147616]; 656: NM_032209, “Homo sapiens hypothetical
protein FLJ21777 (FLJ21777), mRNA”, gi|14149905|ref|NM_032209.1|[14149905]; 657:
NM_032338, “Homo sapiens hypothetical protein MGC14817 (MGC14817), mRNA”,
gi|31543151|ref|NM_032338.2|[31543151]; 658: NM_032348, “Homo sapiens hypothetical
protein MGC3047 (MGC3047), mRNA”, gi|39725651|ref|NM_032348.2|[39725651]; 659:
NM_032389, “Homo sapiens zinc finger protein 289, ID1 regulated (ZNF289), mRNA”,
gi|31543982|ref|NM_032389.2|[31543982]; 660: NM_032842, “Homo sapiens hypothetical
protein FLJ14803 (FLJ14803), mRNA”, gi|14249557|ref|NM_032842.1|[14249557]; 661:
NM_033100, “Homo sapiens protocadherin 21 (PCDH21), mRNA”,
gi|16933564|ref|NM_033100.1|[16933564]; 662: NM_033184, “Homo sapiens keratin
associated protein 2-4 (KRTAP2-4), mRNA”, gi|15743557|ref|NM_033184.2|[15743557]; 663:
NM_080284, “Homo sapiens ATP-binding cassette, sub-family A (ABC1), member 6
(ABCA6),”, “transcript variant 1, mRNA”, gi|27436952|ref|NM_080284.2|[27436952]; 664:
NM_080603, “Homo sapiens zinc finger, SWIM domain containing 1 (ZSWIM1), mRNA”,
gi|29126221|ref|NM_080603.2|[29126221]; 665: NM_130463, “Homo sapiens ATPase, H+
transporting, lysosomal 13 kDa, V1 subunit G isoform 2”, “(ATP6V1G2), transcript variant 1,
mRNA”, gi|20357536|ref|NM_130463.2|[20357536]; 666: NM_138340, “Homo sapiens
abhydrolase domain containing 3 (ABHD3), mRNA”,
gi|34304337|ref|NM_138340.3|[34304337]; 667: NM_138967, “Homo sapiens secretory carrier
membrane protein 5 (SCAMP5), mRNA”, gi|42544128|ref|NM_138967.2|[42544128]; 668:
NM_144563, Homo sapiens ribose 5-phosphate isomerase A (ribose 5-phosphate epimerase),
“(RPIA), mRNA”, gi|21389336|ref|NM_144563.1|[21389336]; 669: NM_144718, “Homo
sapiens hypothetical protein AY099107 (LOC152185), mRNA”,
gi|40255074|ref|NM_144718.2|[40255074]; 670: NM_145021, “Homo sapiens c-mir, cellular
modulator of immune recognition (MIR), mRNA”, gi|34222177|ref|NM_145021.2|[34222177];
671: NM_145804, “Homo sapiens ankyrin repeat and BTB (POZ) domain containing 2
(ABTB2), mRNA”, gi|21956638|ref|NM_145804.1|[21956638]; 672: NM_152344, “Homo
sapiens hypothetical protein FLJ30656 (FLJ30656), mRNA”,
gi|22748746|ref|NM_152344.1|[22748746]; 673: NM_152470, “Homo sapiens hypothetical
protein FLJ34218 (FLJ34218), mRNA”, gi|22748990|ref|NM_152470.1|[22748990]; 674:
NM_153045, “Homo sapiens DKFZp547P234 protein (DKFZp547P234), mRNA”,
gi|33356141|ref|NM_153045.2|[33356141]; 675: NM_153354, “Homo sapiens hypothetical
protein MGC33214 (MGC33214), mRNA”, gi|34222213|ref|NM_153354.2|[34222213]; 676:
NM_174975, “Homo sapiens SEC14-like 3 (S. cerevisiae) (SEC14L3), mRNA”,
gi|30410717|ref|NM_174975.2|[30410717]; 677: NM_174977, “Homo sapiens SEC14-like 4 (S. cerevisiae)
(SEC14L4), mRNA”, gi|30410718|ref|NM_174977.2|[30410718]; 678: NM_175852,
“Homo sapiens taxilin (DKFZp451J0118), mRNA”, gi|39725959|ref|NM_175852.3|[39725959],

TABLE 12
Genes having an Gabpa binding site motif

1: NM_000028, “Homo sapiens amylo-1, 6-glucosidase, 4-alpha-glucanotransferase (glycogen”,
“debranching enzyme, glycogen storage disease type III) (AGL), transcript variant”, “4, mRNA”,
gi|4557274|ref|NM_000028.1|[4557274]; 2: NM_000029, “Homo sapiens angiotensinogen
(serine (or cysteine) proteinase inhibitor, clade A”, “(alpha-1 antiproteinase, antitrypsin),
member 8) (AGT), mRNA”, gi|4557286|ref|NM_000029.1|[4557286]; 3: NM_000033, “Homo
sapiens ATP-binding cassette, sub-family D (ALD), member 1 (ABCD1), mRNA”,
gi|7262392|ref|NM_000033.2|[7262392]; 4: NM_000040, “Homo sapiens apolipoprotein C-III
(APOC3), mRNA”, gi|4557322|ref|NM_000040.1|[4557322]; 5: NM_000045, “Homo sapiens
arginase, liver (ARG1), mRNA”, gi|10947138|ref|NM_000045.2|[10947138]; 6: NM_000049,
“Homo sapiens aspartoacylase (aminoacylase 2, Canavan disease) (ASPA), mRNA”,
gi|4557334|ref|NM_000049.1|[4557334]; 7: NM_000053, “Homo sapiens ATPase, Cu++
transporting, beta polypeptide (Wilson disease)”, “(ATP7B), mRNA”,
gi|4502322|ref|NM_000053.1|[4502322]; 8: NM_000055, “Homo sapiens butyrylcholinesterase
(BCHE), mRNA”, gi|4557350|ref|NM_000055.1|[4557350]; 9: NM_000057, “Homo sapiens
Bloom syndrome (BLM), mRNA”, gi|4557364|ref|NM_000057.1|[4557364]; 10: NM_000063,
“Homo sapiens complement component 2 (C2), mRNA”,
gi|20631970|ref|NM_000063.3|[20631970]; 11: NM_000069, “Homo sapiens calcium channel,
voltage-dependent, L type, alpha 1S subunit”, “(CACNA1S), mRNA”,
gi|4557400|ref|NM_000069.1|[4557400]; 12: NM_000075, “Homo sapiens cyclin-dependent
kinase 4 (CDK4), mRNA”, gi|16936531|ref|NM_000075.2|[16936531]; 13: NM_000092,
“Homo sapiens collagen, type IV, alpha 4 (COL4A4), mRNA”,
gi|15890083|ref|NM_000092.2|[15890083]; 14: NM_000103, “Homo sapiens cytochrome P450,
family 19, subfamily A, polypeptide 1 (CYP19A1),”, “transcript variant 1, mRNA”,
gi|13904857|ref|NM_000103.2|[13904857]; 15: NM_000110, “Homo sapiens dihydropyrimidine
dehydrogenase (DPYD), mRNA”, gi|4557874|ref|NM_000110.2|[4557874]; 16: NM_000122,
“Homo sapiens excision repair cross-complementing rodent repair deficiency,”,
“complementation group 3 (xeroderma pigmentosum group B complementing) (ERCC3),”,
mRNA, gi|4557562|ref|NM_000122.1|[4557562]; 17: NM_000123, “Homo sapiens excision
repair cross-complementing rodent repair deficiency,”, “complementation group 5 (xeroderma
pigmentosum, complementation group G”, “(Cockayne syndrome)) (ERCC5), mRNA”,
gi|4503600|ref|NM_000123.1|[4503600]; 18: NM_000124, “Homo sapiens excision repair
cross-complementing rodent repair deficiency,”, “complementation group 6 (ERCC6), mRNA”,
gi|4557564|ref|NM_000124.1|[4557564]; 19: NM_000127, “Homo sapiens exostoses (multiple)
1 (EXT1), mRNA”, gi|4557570|ref|NM_000127.1|[4557570]; 20: NM_000129, “Homo sapiens
coagulation factor XIII, A1 polypeptide (F13A1), mRNA”,
gi|9961355|ref|NM_000129.2|[9961355]; 21: NM_000147, “Homo sapiens fucosidase, alpha-L-
1, tissue (FUCA1), mRNA”, gi|24475878|ref|NM_000147.2|[24475878]; 22: NM_000148,
Homo sapiens fucosyltransferase 1 (galactoside 2-alpha-L-fucosyltransferase), “(FUT1),
mRNA”, gi|4503804|ref|NM_000148.1|[4503804]; 23: NM_000158, “Homo sapiens glucan
(1,4-alpha-), branching enzyme 1 (glycogen branching enzyme,”, “Andersen disease, glycogen
storage disease type IV) (GBE1), mRNA”, gi|4557618|ref|NM_000158.1|[4557618]; 24:
NM_000164, “Homo sapiens gastric inhibitory polypeptide receptor (GIPR), mRNA”,
gi|4503998|ref|NM_000164.1|[4503998]; 25: NM_000168, Homo sapiens GLI-Kruppel family
member GLI3 (Greig cephalopolysyndactyly, “syndrome) (GLI3), mRNA”,
gi|13518031|ref|NM_000168.2|[13518031]; 26: NM_000174, “Homo sapiens glycoprotein IX
(platelet) (GP9), mRNA”, gi|4504076|ref|NM_000174.1|[4504076]; 27: NM_000183, Homo
sapiens hydroxyacyl-Coenzyme A dehydrogenase/3-ketoacyl-Coenzyme A, “thiolase/enoyl-
Coenzyme A hydratase (trifunctional protein), beta subunit”, “(HADHB), mRNA”,
gi|4504326|ref|NM_000183.1|[4504326]; 28: NM_000188, “Homo sapiens hexokinase 1 (HK1),
nuclear gene encoding mitochondrial protein,”, “transcript variant 1, mRNA”,
gi|4504390|ref|NM_000188.1|[4504390]; 29: NM_000190, “Homo sapiens
hydroxymethylbilane synthase (HMBS), mRNA”, gi|20149499|ref|NM_000190.2|[20149499];
30: NM_000191, Homo sapiens 3-hydroxymethyl-3-methylglutaryl-Coenzyme A lyase,
“(hydroxymethylglutaricaciduria) (HMGCL), mRNA”, gi|4504426|ref|NM_000191.1|[4504426];
31: NM_000193, “Homo sapiens sonic hedgehog homolog (Drosophila) (SHH), mRNA”,
gi|21071042|ref|NM_000193.2|[21071042]; 32: NM_000230, “Homo sapiens leptin (obesity
homolog, mouse) (LEP), mRNA”, gi|4557714|ref|NM_000230.1|[4557714]; 33: NM_000234,
“Homo sapiens ligase I, DNA, ATP-dependent (LIG1), mRNA”,
gi|4557718|ref|NM_000234.1|[4557718]; 34: NM_000248, “Homo sapiens microphthalmia-
associated transcription factor (MITF), transcript”, “variant 4, mRNA”,
gi|38156695|ref|NM_000248.2|[38156695]; 35: NM_000249, “Homo sapiens mutL homolog 1,
colon cancer, nonpolyposis type 2 (E. coli) (MLH1),”, mRNA,
gi|28559089|ref|NM_000249.2|[28559089]; 36: NM_000251, “Homo sapiens mutS homolog 2,
colon cancer, nonpolyposis type 1 (E. coli) (MSH2),”, mRNA,
gi|4557760|ref|NM_000251.1|[4557760]; 37: NM_000254, “Homo sapiens 5-
methyltetrahydrofolate-homocysteine methyltransferase (MTR), mRNA”,
gi|4557764|ref|NM_000254.1|[4557764]; 38: NM_000261, “Homo sapiens myocilin, trabecular
meshwork inducible glucocorticoid response”, “(MYOC), mRNA”,
gi|4557778|ref|NM_000261.1|[4557778]; 39: NM_000274, “Homo sapiens ornithine
aminotransferase (gyrate atrophy) (OAT), nuclear gene”, “encoding mitochondrial protein,
mRNA”, gi|4557808|ref|NM_000274.1|[4557808]; 40: NM_000277, “Homo sapiens
phenylalanine hydroxylase (PAH), mRNA”, gi|4557818|ref|NM_000277.1|[4557818]; 41:
NM_000278, “Homo sapiens paired box gene 2 (PAX2), transcript variant b, mRNA”,
gi|34878700|ref|NM_000278.2|[34878700]; 42: NM_000280, “Homo sapiens paired box gene 6
(aniridia, keratitis) (PAX6), mRNA”, gi|4505614|ref|NM_000280.1|[4505614]; 43: NM_000286,
“Homo sapiens peroxisomal biogenesis factor 12 (PEX12), mRNA”,
gi|4505720|ref|NM_000286.1|[4505720]; 44: NM_000294, “Homo sapiens phosphorylase
kinase, gamma 2 (testis) (PHKG2), mRNA”, gi|4505784|ref|NM_000294.1|[4505784]; 45:
NM_000297, “Homo sapiens polycystic kidney disease 2 (autosomal dominant) (PKD2),
mRNA”, gi|33286447|ref|NM_000297.2|[33286447]; 46: NM_000300, “Homo sapiens
phospholipase A2, group IIA (platelets, synovial fluid) (PLA2G2A),”, mRNA,
gi|20149501|ref|NM_000300.2|[20149501]; 47: NM_000302, “Homo sapiens procollagen-
lysine, 2-oxoglutarate 5-dioxygenase (lysine”, “hydroxylase, Ehlers-Danlos syndrome type VI)
(PLOD), mRNA”, gi|32307143|ref|NM_000302.2|[32307143]; 48: NM_000304, “Homo sapiens
peripheral myelin protein 22 (PMP22), transcript variant 1, mRNA”,
gi|24430161|ref|NM_000304.2|[24430161]; 49: NM_000308, Homo sapiens protective protein
for beta-galactosidase (galactosialidosis), “(PPGB), mRNA”,
gi|4505988|ref|NM_000308.1|[4505988]; 50: NM_000316, “Homo sapiens parathyroid hormone
receptor 1 (PTHR1), mRNA”, gi|39995096|ref|NM_000316.2|[39995096]; 51: NM_000317,
“Homo sapiens 6-pyruvoyltetrahydropterin synthase (PTS), mRNA”,
gi|4506330|ref|NM_000317.1|[4506330]; 52: NM_000318, “Homo sapiens peroxisomal
membrane protein 3, 35 kDa (Zellweger syndrome) (PXMP3),”, mRNA,
gi|4506342|ref|NM_000318.1|[4506342]; 53: NM_000328, “Homo sapiens retinitis pigmentosa
GTPase regulator (RPGR), mRNA”, gi|4506580|ref|NM_000328.1|[4506580]; 54: NM_000347,
“Homo sapiens spectrin, beta, erythrocytic (includes spherocytosis, clinical type”, “I) (SPTB),
mRNA”, gi|22507315|ref|NM_000347.3|[22507315]; 55: NM_000348, “Homo sapiens steroid-
5-alpha-reductase, alpha polypeptide 2 (3-oxo-5”, “alpha-steroid delta 4-dehydrogenase alpha 2)
(SRD5A2), mRNA”, gi|39812446|ref|NM_000348.2|[39812446]; 56: NM_000359, “Homo
sapiens transglutaminase 1 (K polypeptide epidermal type I,”, “protein-glutamine-gamma-
glutamyltransferase) (TGM1), mRNA”, gi|4507474|ref|NM_000359.1|[4507474]; 57:
NM_000364, “Homo sapiens troponin T2, cardiac (TNNT2), mRNA”,
gi|4507626|ref|NM_000364.1|[4507626]; 58: NM_000368, “Homo sapiens tuberous sclerosis 1
(TSC1), mRNA”, gi|24475626|ref|NM_000368.2|[24475626]; 59: NM_000375, Homo sapiens
uroporphyrinogen III synthase (congenital erythropoietic porphyria), “(UROS), mRNA”,
gi|4557872|ref|NM_000375.1|[4557872]; 60: NM_000383, Homo sapiens autoimmune regulator
(autoimmune polyendocrinopathy candidiasis, “ectodermal dystrophy) (AIRE), transcript variant
AIRE-1, mRNA”, gi|4557290|ref|NM_000383.1|[4557290]; 61: NM_000387, “Homo sapiens
solute carrier family 25 (carnitine/acylcarnitine translocase),”, “member 20 (SLC25A20), nuclear
gene encoding mitochondrial protein, mRNA”, gi|6006040|ref|NM_000387.2|[6006040]; 62:
NM_000389, “Homo sapiens cyclin-dependent kinase inhibitor 1A (p21, Cip1) (CDKN1A),”,
“transcript variant 1, mRNA”, gi|7978496|ref|NM_000389.2|[17978496]; 63: NM_000396,
“Homo sapiens cathepsin K (pycnodysostosis) (CTSK), mRNA”,
gi|23110958|ref|NM_000396.2|[23110958]; 64: NM_000399, “Homo sapiens early growth
response 2 (Krox-20 homolog, Drosophila) (EGR2), mRNA”,
gi|9845523|ref|NM_000399.2|[9845523]; 65: NM_000402, “Homo sapiens glucose-6-phosphate
dehydrogenase (G6PD), nuclear gene encoding”, “mitochondrial protein, mRNA”,
gi|21614519|ref|NM_000402.2|[21614519]; 66: NM_000403, “Homo sapiens galactose-4-
epimerase, UDP (GALE), mRNA”, gi|9945333|ref|NM_000403.2|[9945333]; 67: NM_000429,
“Homo sapiens methionine adenosyltransferase I, alpha (MAT1A), mRNA”,
gi|4557736|ref|NM_000429.1|[4557736]; 68: NM_000434, “Homo sapiens sialidase 1
(lysosomal sialidase) (NEU1), mRNA”, gi|40806202|ref|NM_000434.2|[40806202]; 69:
NM_000474, Homo sapiens twist homolog 1 (acrocephalosyndactyly 3; Saethre-Chotzen
syndrome), “(Drosophila) (TWIST1), mRNA”, gi|17978464|ref|NM_000474.2|[17978464]; 70:
NM_000483, “Homo sapiens apolipoprotein C-II (APOC2), mRNA”,
gi|32130517|ref|NM_000483.3|[32130517]; 71: NM_000499, “Homo sapiens cytochrome P450,
family 1, subfamily A, polypeptide 1 (CYP1A1),”, mRNA,
gi|13325053|ref|NM_000499.2|[13325053]; 72: NM_000503, “Homo sapiens eyes absent
homolog 1 (Drosophila) (EYA1), transcript variant 3,”, mRNA,
gi|26667213|ref|NM_000503.3|[26667213]; 73: NM_000512, “Homo sapiens galactosamine (N-
acetyl)-6-sulfate sulfatase (Morquio syndrome,”, “mucopolysaccharidosis type IVA) (GALNS),
mRNA”, gi|9945384|ref|NM_000512.2|[9945384]; 74: NM_000514, “Homo sapiens glial cell
derived neurotrophic factor (GDNF), transcript variant”, “1, mRNA”,
gi|40549401|ref|NM_000514.2|[40549401]; 75: NM_000524, “Homo sapiens 5-
hydroxytryptamine (serotonin) receptor 1A (HTR1A), mRNA”,
gi|4504530|ref|NM_000524.1|[4504530]; 76: NM_000526, “Homo sapiens keratin 14
(epidermolysis bullosa simplex, Dowling-Meara, Koebner)”, “(KRT14), mRNA”,
gi|15431309|ref|NM_000526.3|[15431309]; 77: NM_000528, “Homo sapiens mannosidase,
alpha, class 2B, member 1 (MAN2B1), mRNA”, gi|10834967|ref|NM_000528.1|[10834967]; 78:
NM_000534, “Homo sapiens PMS1 postmeiotic segregation increased 1 (S. cerevisiae)
(PMS1),”, mRNA, gi|11496979|ref|NM_000534.2|[11496979]; 79: NM_000547, “Homo sapiens
thyroid peroxidase (TPO), transcript variant 1, mRNA”,
gi|28558981|ref|NM_000547.3|[28558981]; 80: NM_000548, “Homo sapiens tuberous sclerosis
2 (TSC2), transcript variant 1, mRNA”, gi|10938006|ref|NM_000548.2|[10938006]; 81:
NM_000581, “Homo sapiens glutathione peroxidase 1 (GPX1), transcript variant 1, mRNA”,
gi|41406083|ref|NM_000581.2|[41406083]; 82: NM_000582, “Homo sapiens secreted
phosphoprotein 1 (osteopontin, bone sialoprotein I, early”, “T-lymphocyte activation 1) (SPP1),
mRNA”, gi|38146097|ref|NM_000582.2|[38146097]; 83: NM_000585, “Homo sapiens
interleukin 15 (IL15), transcript variant 3, mRNA”, gi|26787979|ref|NM_000585.2|[26787979];
84: NM_000588, “Homo sapiens interleukin 3 (colony-stimulating factor, multiple) (IL3),
mRNA”, gi|28416914|ref|NM_000588.3|[28416914]; 85: NM_000592, “Homo sapiens
complement component 4B (C4B), mRNA”, gi|14577920|ref|NM_000592.3|[14577920]; 86:
NM_000593, “Homo sapiens transporter 1, ATP-binding cassette, sub-family B (MDR/TAP)
(TAP1),”, mRNA, gi|24797159|ref|NM_000593.4|[24797159]; 87: NM_000594, “Homo sapiens
tumor necrosis factor (TNF superfamily, member 2) (TNF), mRNA”,
gi|25952110|ref|NM_000594.2|[25952110]; 88: NM_000595, “Homo sapiens lymphotoxin
alpha (TNF superfamily, member 1) (LTA), mRNA”, gi|6806892|ref|NM_000595.2|[6806892];
89: NM_000600, “Homo sapiens interleukin 6 (interferon, beta 2) (IL6), mRNA”,
gi|10834983|ref|NM_000600.1|[10834983]; 90: NM_000603, “Homo sapiens nitric oxide
synthase 3 (endothelial cell) (NOS3), mRNA”, gi|40254421|ref|NM_000603.2|[40254421]; 91:
NM_000606, “Homo sapiens complement component 8, gamma polypeptide (C8G), mRNA”,
gi|4557392|ref|NM_000606.1|[4557392]; 92: NM_000623, “Homo sapiens bradykinin receptor
B2 (BDKRB2), mRNA”, gi|17352499|ref|NM_000623.2|[17352499]; 93: NM_000626, “Homo
sapiens CD79B antigen (immunoglobulin-associated beta) (CD79B), transcript”, “variant 1,
mRNA”, gi|11038673|ref|NM_000626.1|[11038673]; 94: NM_000628, “Homo sapiens
interleukin 10 receptor, beta (IL10RB), mRNA”, gi|24430214|ref|NM_000628.3|[24430214]; 95:
NM_000635, “Homo sapiens regulatory factor X, 2 (influences HLA class II expression)
(RFX2),”, “transcript variant 1, mRNA”, gi|19743880|ref|NM_000635.2|[19743880]; 96:
NM_000637, “Homo sapiens glutathione reductase (GSR), mRNA”,
gi|10835188|ref|NM_000637.1|[10835188]; 97: NM_000638, “Homo sapiens vitronectin (serum
spreading factor, somatomedin B, complement”, “S-protein) (VTN), mRNA”,
gi|18201910|ref|NM_000638.2|[18201910]; 98: NM_000661, “Homo sapiens ribosomal protein
L9 (RPL9), mRNA”, gi|15431302|ref|NM_000661.2|[15431302]; 99: NM_000673, “Homo
sapiens alcohol dehydrogenase 7 (class IV), mu or sigma polypeptide (ADH7),”, mRNA,
gi|11496969|ref|NM_000673.2|[11496969]; 100: NM_000679, “Homo sapiens adrenergic,
alpha-1B-, receptor (ADRA1B), mRNA”, gi|15451783|ref|NM_000679.2|[15451783]; 101:
NM_000681, “Homo sapiens adrenergic, alpha-2A-, receptor (ADRA2A), mRNA”,
gi|15718669|ref|NM_000681.2|[15718669]; 102: NM_000682, “Homo sapiens adrenergic,
alpha-2B-, receptor (ADRA2B), mRNA”, gi|33598959|ref|NM_000682.3|[33598959]; 103:
NM_000684, “Homo sapiens adrenergic, beta-1-, receptor (ADRB1), mRNA”,
gi|4557264|ref|NM_000684.1|[4557264]; 104: NM_000687, “Homo sapiens S-
adenosylhomocysteine hydrolase (AHCY), mRNA”, gi|9951914|ref|NM_000687.1|[9951914];
105: NM_000688, “Homo sapiens aminolevulinate, delta-, synthase 1 (ALAS1), transcript
variant 1,”, mRNA, gi|40316942|ref|NM_000688.4|[40316942]; 106: NM_000697, “Homo
sapiens arachidonate 12-lipoxygenase (ALOX12), mRNA”,
gi|4502050|ref|NM_000697.1|[4502050]; 107: NM_000721, “Homo sapiens calcium channel,
voltage-dependent, alpha 1E subunit (CACNALE),”, mRNA,
gi|4502528|ref|NM_000721.1|[4502528]; 108: NM_000747, “Homo sapiens cholinergic
receptor, nicotinic, beta polypeptide 1 (muscle)”, “(CHRNB1), mRNA”,
gi|41327725|ref|NM_000747.2|[41327725]; 109: NM_000751, “Homo sapiens cholinergic
receptor, nicotinic, delta polypeptide (CHRND), mRNA”,
gi|4557460|ref|NM_000751.1|[4557460]; 110: NM_000760, “Homo sapiens colony stimulating
factor 3 receptor (granulocyte) (CSF3R),”, “transcript variant 1, mRNA”,
gi|27437046|ref|NM_000760.2|[27437046]; 111: NM_000781, “Homo sapiens cytochrome
P450, family 11, subfamily A, polypeptide 1 (CYP11A1),”, “nuclear gene encoding
mitochondrial protein, mRNA”, gi|4503188|ref|NM_000781.1|[4503188]; 112: NM_000782,
“Homo sapiens cytochrome P450, family 24, subfamily A, polypeptide 1 (CYP24A1),”, “nuclear
gene encoding mitochondrial protein, mRNA”, gi|13904862|ref|NM_000782.2|[13904862]; 113:
NM_000784, “Homo sapiens cytochrome P450, family 27, subfamily A, polypeptide 1
(CYP27A1),”, “nuclear gene encoding mitochondrial protein, mRNA”,
gi|13904863|ref|NM_000784.2|[13904863]; 114: NM_000785, “Homo sapiens cytochrome
P450, family 27, subfamily B, polypeptide 1 (CYP27B1),”, “nuclear gene encoding
mitochondrial protein, mRNA”, gi|13904864|ref|NM_000785.2|[13904864]; 115: NM_000794,
“Homo sapiens dopamine receptor D1 (DRD1), mRNA”,
gi|16445404|ref|NM_000794.2|[16445404]; 116: NM_000798, “Homo sapiens dopamine
receptor D5 (DRD5), mRNA”, gi|34328907|ref|NM_000798.3|[34328907]; 117: NM_000806,
“Homo sapiens gamma-aminobutyric acid (GABA) A receptor, alpha 1 (GABRA1), mRNA”,
gi|38327553|ref|NM_000806.3|[38327553]; 118: NM_000809, “Homo sapiens gamma-
aminobutyric acid (GABA) A receptor, alpha 4 (GABRA4), mRNA”,
gi|34452722|ref|NM_000809.2|[34452722]; 119: NM_000813, “Homo sapiens gamma-
aminobutyric acid (GABA) A receptor, beta 2 (GABRB2),”, “transcript variant 2, mRNA”,
gi|4503864|ref|NM_000813.1|[4503864]; 120: NM_000831, “Homo sapiens glutamate receptor,
ionotropic, kainate 3 (GRIK3), mRNA”, gi|28605144|ref|NM_000831.2|[28605144]; 121:
NM_000835, “Homo sapiens glutamate receptor, ionotropic, N-methyl D-aspartate 2C
(GRIN2C),”, mRNA, gi|6006004|ref|NM_000835.2|[6006004]; 122: NM_000839, “Homo
sapiens glutamate receptor, metabotropic 2 (GRM2), mRNA”,
gi|4504136|ref|NM_000839.1|[4504136]; 123: NM_000841, “Homo sapiens glutamate receptor,
metabotropic 4 (GRM4), mRNA”, gi|4504140|ref|NM_000841.1|[4504140]; 124: NM_000849,
“Homo sapiens glutathione S-transferase M3 (brain) (GSTM3), mRNA”,
gi|39995110|ref|NM_000849.3|[39995110]; 125: NM_000863, “Homo sapiens 5-
hydroxytryptamine (serotonin) receptor 1B (HTR1B), mRNA”,
gi|4504532|ref|NM_000863.1|[4504532]; 126: NM_000880, “Homo sapiens interleukin 7 (IL7),
mRNA”, gi|28610152|ref|NM_000880.2|[28610152]; 127: NM_000883, “Homo sapiens IMP
(inosine monophosphate) dehydrogenase 1 (IMPDH1), transcript”, “variant 1, mRNA”,
gi|34328929|ref|NM_000883.2|[34328929]; 128: NM_000894, “Homo sapiens luteinizing
hormone beta polypeptide (LHB), mRNA”, gi|15431286|ref|NM_000894.2|[15431286]; 129:
NM_000901, “Homo sapiens nuclear receptor subfamily 3, group C, member 2 (NR3C2),
mRNA”, gi|4505198|ref|NM_000901.1|[4505198]; 130: NM_000905, “Homo sapiens
neuropeptide Y (NPY), mRNA”, gi|31542152|ref|NM_000905.2|[31542152]; 131: NM_000915,
“Homo sapiens oxytocin, prepro-(neurophysin I) (OXT), mRNA”,
gi|12707574|ref|NM_000915.2|[12707574]; 132: NM_000932, “Homo sapiens phospholipase C,
beta 3 (phosphatidylinositol-specific) (PLCB3),”, mRNA,
gi|11386138|ref|NM_000932.1|[11386138]; 133: NM_000939, Homo sapiens
proopiomelanocortin (adrenocorticotropin/beta-lipotropin/, alpha-melanocyte stimulating
hormone/beta-melanocyte stimulating hormone/, “beta-endorphin) (POMC), mRNA”,
gi|4505948|ref|NM_000939.1|[4505948]; 134: NM_000951, “Homo sapiens proline-rich Gla
(G-carboxyglutamic acid) polypeptide 2 (PRRG2),”, mRNA,
gi|4506136|ref|NM_000951.1|[4506136]; 135: NM_000963, Homo sapiens prostaglandin-
endoperoxide synthase 2 (prostaglandin G/H synthase, “and cyclooxygenase) (PTGS2), mRNA”,
gi|4506264|ref|NM_000963.1|[4506264]; 136: NM_000970, “Homo sapiens ribosomal protein
L6 (RPL6), mRNA”, gi|16753226|ref|NM_000970.2|[16753226]; 137: NM_000973, “Homo
sapiens ribosomal protein L8 (RPL8), transcript variant 1, mRNA”,
gi|15431304|ref|NM_000973.2|[15431304]; 138: NM_000975, “Homo sapiens ribosomal
protein L11 (RPL11), mRNA”, gi|15431289|ref|NM_000975.2|[15431289]; 139: NM_000980,
“Homo sapiens ribosomal protein L18a (RPL18A), mRNA”,
gi|15431299|ref|NM_000980.2|[15431299]; 140: NM_000981, “Homo sapiens ribosomal
protein L19 (RPL19), mRNA”, gi|17158042|ref|NM_000981.2|[17158042]; 141: NM_000982,
“Homo sapiens ribosomal protein L21 (RPL21), mRNA”,
gi|18104947|ref|NM_000982.2|[18104947]; 142: NM_000993, “Homo sapiens ribosomal
protein L31 (RPL31), mRNA”, gi|15812219|ref|NM_000993.2|[15812219]; 143: NM_000994,
“Homo sapiens ribosomal protein L32 (RPL32), mRNA”,
gi|15812220|ref|NM_000994.2|[15812220]; 144: NM_000995, “Homo sapiens ribosomal
protein L34 (RPL34), transcript variant 1, mRNA”, gi|16117786|ref|NM_000995.2|[16117786];
145: NM_000997, “Homo sapiens ribosomal protein L37 (RPL37), mRNA”,
gi|16306560|ref|NM_000997.2|[16306560]; 146: NM_001000, “Homo sapiens ribosomal
protein L39 (RPL39), mRNA”, gi|16306563|ref|NM_001000.2|[16306563]; 147: NM_001001,
“Homo sapiens ribosomal protein L36a-like (RPL36AL), mRNA”,
gi|34335143|ref|NM_001001.3|[34335143]; 148: NM_001003, “Homo sapiens ribosomal
protein, large, P1 (RPLP1), mRNA”, gi|16905511|ref|NM_001003.2|[16905511]; 149:
NM_001009, “Homo sapiens ribosomal protein S5 (RPS5), mRNA”,
gi|13904869|ref|NM_001009.2|[13904869]; 150: NM_001018, “Homo sapiens ribosomal
protein S15 (RPS15), mRNA”, gi|14591911|ref|NM_001018.2|[14591911]; 151: NM_001019,
“Homo sapiens ribosomal protein S15a (RPS15A), mRNA”,
gi|34335150|ref|NM_001019.3|[34335150]; 152: NM_001026, “Homo sapiens ribosomal
protein S24 (RPS24), transcript variant 2, mRNA”, gi|14916502|ref|NM_001026.2|[14916502];
153: NM_001028, “Homo sapiens ribosomal protein S25 (RPS25), mRNA”,
gi|14591916|ref|NM_001028.2|[14591916]; 154: NM_001029, “Homo sapiens ribosomal
protein S26 (RPS26), mRNA”, gi|15011935|ref|NM_001029.2|[15011935]; 155: NM_001030,
“Homo sapiens ribosomal protein S27 (metallopanstimulin 1) (RPS27), mRNA”,
gi|1501937|ref|NM_001030.2|[15011937]; 156: NM_001031, “Homo sapiens ribosomal
protein S28 (RPS28), mRNA”, gi|15011938|ref|NM_001031.2|[15011938]; 157: NM_001040,
“Homo sapiens sex hormone-binding globulin (SHBG), mRNA”,
gi|7382459|ref|NM_001040.2|[7382459]; 158: NM_001046, “Homo sapiens solute carrier
family 12 (sodium/potassium/chloride transporters),”, “member 2 (SLC12A2), mRNA”,
gi|38569461|ref|NM_001046.2|[38569461]; 159: NM_101049, “Homo sapiens somatostatin
receptor 1 (SSTR1), mRNA”, gi|33946330|ref|NM_001049.2|[33946330]; 160: NM_001051,
“Homo sapiens somatostatin receptor 3 (SSTR3), mRNA”,
gi|4557860|ref|NM_001051.1|[4557860]; 161: NM_001057, “Homo sapiens tachykinin receptor
2 (TACR2), mRNA”, gi|4507344|ref|NM_001057.1|[4507344]; 162: NM_001068, “Homo
sapiens topoisomerase (DNA) II beta 180 kDa (TOP2B), mRNA”,
gi|19913407|ref|NM_001068.2|[19913407]; 163: NM_001083, “Homo sapiens
phosphodiesterase 5A, cGMP-specific (PDE5A), transcript variant 1,”, mRNA,
gi|15812210|ref|NM_001083.2|[15812210]; 164: NM_001087, “Homo sapiens angio-associated,
migratory cell protein (AAMP), mRNA”, gi|4557228|ref|NM_001087.1|[4557228]; 165:
NM_001090, “Homo sapiens ATP-binding cassette, sub-family F (GCN20), member 1
(ABCF1), mRNA”, gi|10947134|ref|NM_001090.1|[10947134]; 166: NM_001094, “Homo
sapiens amiloride-sensitive cation channel 1, neuronal (degenerin) (ACCN1),”, “transcript
variant 2, mRNA”, gi|34452696|ref|NM_001094.4|[34452696]; 167: NM_001098, “Homo
sapiens aconitase 2, mitochondrial (ACO2), nuclear gene encoding”, “mitochondrial protein,
mRNA”, gi|4501866|ref|NM_001098.1|[4501866]; 168: NM_001099, “Homo sapiens acid
phosphatase, prostate (ACPP), mRNA”, gi|6382063|ref|NM_001099.2|[6382063]; 169:
NM_001104, “Homo sapiens actinin, alpha 3 (ACTN3), mRNA”,
gi|4557240|ref|NM_001104.1|[4557240]; 170: NM_001105, “Homo sapiens activin A receptor,
type I (ACVR1), mRNA”, gi|10862690|ref|NM_001105.2|[10862690]; 171: NM_001117,
“Homo sapiens adenylate cyclase activating polypeptide 1 (pituitary) (ADCYAP1),”, mRNA,
gi|10947062|ref|NM_001117.2|[10947062]; 172: NM_001120, “Homo sapiens tetracycline
transporter-like protein (TETRAN), mRNA”, gi|20127439|ref|NM_001120.2|[20127439]; 173:
NM_001124, “Homo sapiens adrenomedullin (ADM), mRNA”,
gi|4501944|ref|NM_001124.1|[4501944]; 174: NM_001125, “Homo sapiens ADP-
ribosylarginine hydrolase (ADPRH), mRNA”, gi|40549393|ref|NM_001125.2|[40549393]; 175:
NM_001126, “Homo sapiens adenylosuccinate synthase (ADSS), mRNA”,
gi|34577062|ref|NM_001126.2|[34577062]; 176: NM_001127, “Homo sapiens adaptor-related
protein complex 1, beta 1 subunit (AP1B1),”, “transcript variant 1, mRNA”,
gi|22027650|ref|NM_001127.2|[22027650]; 177: NM_001129, “Homo sapiens AE binding
protein 1 (AEBP1), mRNA”, gi|4755145|ref|NM_001129.2|[4755145]; 178: NM_001138,
“Homo sapiens agouti related protein homolog (mouse) (AGRP), transcript variant”, “1, mRNA”,
gi|4501994|ref|NM_001138.1|[4501994]; 179: NM_001151, Homo sapiens solute carrier family
25 (mitochondrial carrier; adenine nucleotide, “translocator), member 4 (SLC25A4), mRNA”,
gi|4502096|ref|NM_001151.1|[4502096]; 180: NM_001158, “Homo sapiens amine oxidase,
copper containing 2 (retina-specific) (AOC2),”, “transcript variant 1, mRNA”,
gi|6806880|ref|NM_001158.2|[6806880]; 181: NM_001161, Homo sapiens nudix (nucleoside
diphosphate linked moiety X)-type motif 2, “(NUDT2), transcript variant 1, mRNA”,
gi|22265329|ref|NM_001161.3|[22265329]; 182: NM_001164, “Homo sapiens amyloid beta
(A4) precursor protein-binding, family B, member 1”, “(Fe65) (APBB1), transcript variant 1,
mRNA”, gi|22035552|ref|NM_001164.2|[22035552]; 183: NM_001166, “Homo sapiens
baculoviral IAP repeat-containing 2 (BIRC2), mRNA”,
gi|41349435|ref|NM_001166.3|[41349435]; 184: NM_001170, “Homo sapiens aquaporin 7
(AQP7), mRNA”, gi|4502186|ref|NM_001170.1|[4502186]; 185: NM_001188, “Homo sapiens
BCL2-antagonist/killer 1 (BAK1), mRNA”, gi|33457353|ref|NM_001188.2|[33457353]; 186:
NM_001197, “Homo sapiens BCL2-interacting killer (apoptosis-inducing) (BIK), mRNA”,
gi|21536418|ref|NM_001197.3|[21536418]; 187: NM_001211, Homo sapiens BUB1 budding
uninhibited by benzimidazoles 1 homolog beta (yeast), “(BUB1B), mRNA”,
gi|20149508|ref|NM_001211.3|[20149508]; 188: NM_001231, “Homo sapiens calsequestrin 1
(fast-twitch, skeletal muscle) (CASQ1), nuclear”, “gene encoding mitochondrial protein,
mRNA”, gi|21536273|ref|NM_001231.2|[21536273]; 189: NM_001237, “Homo sapiens cyclin
A2 (CCNA2), mRNA”, gi|16950653|ref|NM_001237.2|[16950653]; 190: NM_001239, “Homo
sapiens cyclin H (CCNH), mRNA”, gi|17738313|ref|NM_001239.2|[17738313]; 191:
NM_001242, “Homo sapiens tumor necrosis factor receptor superfamily, member 7
(TNFRSF7),”, mRNA, gi|23510435|ref|NM_001242.3|[23510435]; 192: NM_001246, “Homo
sapiens ectonucleoside triphosphate diphosphohydrolase 2 (ENTPD2), mRNA”,
gi|4557420|ref|NM_001246.1|[4557420]; 193: NM_001255, “Homo sapiens CDC20 cell
division cycle 20 homolog (S. cerevisiae) (CDC20), mRNA”,
gi|4557436|ref|NM_001255.1|[4557436]; 194: NM_001257, “Homo sapiens cadherin 13, H-
cadherin (heart) (CDH13), mRNA”, gi|16507956|ref|NM_001257.2|[16507956]; 195:
NM_001261, “Homo sapiens cyclin-dependent kinase 9 (CDC2-related kinase) (CDK9),
mRNA”, gi|17017983|ref|NM_001261.2|[17017983]; 196: NM_001265, “Homo sapiens caudal
type homeo box transcription factor 2 (CDX2), mRNA”,
gi|24431948|ref|NM_001265.2|[24431948]; 197: NM_001278, “Homo sapiens conserved helix-
loop-helix ubiquitous kinase (CHUK), mRNA”, gi|19923133|ref|NM_001278.2|[19923133]; 198:
NM_001286, “Homo sapiens chloride channel 6 (CLCN6), transcript variant ClC-6a, mRNA”,
gi|4502872|ref|NM_001286.1|[4502872]; 199: NM_001288, “Homo sapiens chloride
intracellular channel 1 (CLIC1), mRNA”, gi|14251208|ref|NM_001288.3|[14251208]; 200:
NM_001291, “Homo sapiens CDC-like kinase 2 (CLK2), transcript variant phclk2/139,
mRNA”, gi|4557476|ref|NM_001291.1|[4557476]; 201: NM_001293, “Homo sapiens chloride
channel, nucleotide-sensitive, 1A (CLNS1A), mRNA”, gi|4502890|ref|NM_001293.1|[4502890];
202: NM_001303, “Homo sapiens COX10 homolog, cytochrome c oxidase assembly protein,
heme A:”, “farnesyltransferase (yeast) (COX10), nuclear gene encoding mitochondrial”,
“protein, mRNA”, gi|17921981|ref|NM_001303.2|[17921981]; 203: NM_001307, “Homo
sapiens claudin 7 (CLDN7), mRNA”, gi|34222214|ref|NM_001307.3|[34222214]; 204:
NM_001311, “Homo sapiens cysteine-rich protein 1 (intestinal) (CRIP1), mRNA”,
gi|39725694|ref|NM_001311.3|[39725694]; 205: NM_001313, “Homo sapiens collapsin
response mediator protein 1 (CRMP1), mRNA”, gi|21359849|ref|NM_001313.2|[21359849];
206: NM_001320, “Homo sapiens casein kinase 2, beta polypeptide (CSNK2B), mRNA”,
gi|26787971|ref|NM_001320.5|[26787971]; 207: NM_001326, “Homo sapiens cleavage
stimulation factor, 3′ pre-RNA, subunit 3, 77 kDa (CSTF3),”, mRNA,
gi|4557494|ref|NM_001326.1|[4557494]; 208: NM_001338, “Homo sapiens coxsackie virus and
adenovirus receptor (CXADR), mRNA”, gi|20149514|ref|NM_001338.2|[20149514]; 209:
NM_001347, “Homo sapiens diacylglycerol kinase, theta 110 kDa (DGKQ), mRNA”,
gi|40806174|ref|NM_001347.2|[40806174]; 210: NM_001362, “Homo sapiens deiodinase,
iodothyronine, type III (DIO3), mRNA”, gi|4503334|ref|NM_001362.1|[4503334]; 211:
NM_001374, “Homo sapiens deoxyribonuclease I-like 2 (DNASE1L2), mRNA”,
gi|41393584|ref|NM_001374.2|[41393584]; 212: NM_001378, “Homo sapiens dynein,
cytoplasmic, intermediate polypeptide 2 (DNCI2), mRNA”,
gi|24307878|ref|NM_001378.1|[24307878]; 213: NM_001382,, ref|NM_001382.2|[42794008];
214: NM_001384, “Homo sapiens DPH2-like 2 (S. cerevisiae) (DPH2L2), transcript variant 1,
mRNA”, gi|41352701|ref|NM_001384.3|[41352701]; 215: NM_001386, “Homo sapiens
dihydropyrimidinase-like 2 (DPYSL2), mRNA”, gi|19923654|ref|NM_001386.3|[19923654];
216: NM_001389, “Homo sapiens Down syndrome cell adhesion molecule (DSCAM), mRNA”,
gi|20127421|ref|NM_001389.2|[20127421]; 217: NM_001395, “Homo sapiens dual specificity
phosphatase 9 (DUSP9), mRNA”, gi|4503420|ref|NM_001395.1|[4503420]; 218: NM_001414,
“Homo sapiens eukaryotic translation initiation factor 2B, subunit 1 alpha, 26 kDa”, “(EIF2B1),
mRNA”, gi|4503502|ref|NM_001414.1|[4503502]; 219: NM_001415, “Homo sapiens
eukaryotic translation initiation factor 2, subunit 3 gamma, 52 kDa”, “(EIF2S3), mRNA”,
gi|21314612|ref|NM_001415.2|[21314612]; 220: NM_001420, “Homo sapiens ELAV
(embryonic lethal, abnormal vision, Drosophila)-like 3 (Hu”, “antigen C) (ELAVL3), mRNA”,
gi|5231299|ref|NM_001420.2|[5231299]; 221: NM_001424, “Homo sapiens epithelial
membrane protein 2 (EMP2), mRNA”, gi|42716292|ref|NM_001424.3|[42716292]; 222:
NM_001425, “Homo sapiens epithelial membrane protein 3 (EMP3), mRNA”,
gi|4503562|ref|NM_001425.1|[4503562]; 223: NM_001426, “Homo sapiens engrailed homolog
1 (EN1), mRNA”, gi|7710118|ref|NM_001426.2|[7710118]; 224: NM_001430, “Homo sapiens
endothelial PAS domain protein 1 (EPAS1), mRNA”,
gi|41327154|ref|NM_001430.3|[41327154]; 225: NM_001433, “Homo sapiens ER to nucleus
signalling 1 (ERN1), mRNA”, gi|4557568|ref|NM_001433.1|[4557568]; 226: NM_001436,
“Homo sapiens fibrillarin (FBL), mRNA”, gi|12056464|ref|NM_001436.2|[12056464]; 227:
NM_001450, “Homo sapiens four and a half LIM domains 2 (FHL2), transcript variant 1,
mRNA”, gi|42403584|ref|NM_001450.3|[42403584]; 228: NM_001451, “Homo sapiens
forkhead box F1 (FOXF1), mRNA”, gi|4503732|ref|NM_001451.1|[4503732]; 229: NM_001454,
“Homo sapiens forkhead box J1 (FOXJ1), mRNA”, gi|4557023|ref|NM_001454.1|[4557023];
230: NM_001467, “Homo sapiens solute carrier family 37 (glycerol-6-phosphate transporter),
member”, “4 (SLC37A4), mRNA”, gi|21361125|ref|NM_001467.2|[21361125]; 231:
NM_001469, “Homo sapiens thyroid autoantigen 70 kDa (Ku antigen) (G22P1), mRNA”,
gi|20070134|ref|NM_001469.2|[20070134]; 232: NM_001481, “Homo sapiens growth arrest-
specific 8 (GAS8), mRNA”, gi|4503916|ref|NM_001481.1|[4503916]; 233: NM_001485,
“Homo sapiens gastrulation brain homeo box 2 (GBX2), mRNA”,
gi|4503940|ref|NM_001485.1|[4503940]; 234: NM_001486, “Homo sapiens glucokinase
(hexokinase 4) regulatory protein (GCKR), mRNA”, gi|30795244|ref|NM_001486.2|[30795244];
235: NM_001487, Homo sapiens GCN5 general control of amino-acid synthesis 5-like 1
(yeast), “(GCN5L1), mRNA”, gi|4503954|ref|NM_001487.1|[4503954]; 236: NM_001491,
“Homo sapiens glucosaminyl (N-acetyl) transferase 2, I-branching enzyme (GCNT2),”,
“transcript variant 2, mRNA”, gi|30061504|ref|NM_001491.2|[30061504]; 237: NM_001501,
“Homo sapiens gonadotropin-releasing hormone 2 (GNRH2), transcript variant 1,”, mRNA,
gi|4504056|ref|NM_001501.1|[4504056]; 238: NM_001511, Homo sapiens chemokine
(C—X—C motif) ligand 1 (melanoma growth stimulating, “activity, alpha) (CXCL1), mRNA”,
gi|4504152|ref|NM_001511.1|[4504152]; 239: NM_001513, Homo sapiens glutathione
transferase zeta 1 (maleylacetoacetate isomerase), “(GSTZ1), transcript variant 3, mRNA”,
gi|22202621|ref|NM_001513.2|[22202621]; 240: NM_001516, “Homo sapiens general
transcription factor IIH, polypeptide 3, 34 kDa (GTF2H3),”, mRNA,
gi|28376643|ref|NM_001516.3|[28376643]; 241: NM_001517,, ref|NM_001517.3|[34222289],
This record was temporarily removed by RefSeq staff for additional review.,, 242: NM_001523,
“Homo sapiens hyaluronan synthase 1 (HAS1), mRNA”,
gi|4504338|ref|NM_001523.1|[4504338]; 243: NM_001527, “Homo sapiens histone deacetylase
2 (HDAC2), mRNA”, gi|4557640|ref|NM_001527.1|[4557640]; 244: NM_001528, “Homo
sapiens HGF activator (HGFAC), mRNA”, gi|32455241|ref|NM_001528.2|[32455241]; 245:
NM_001536, “Homo sapiens HMT1 hnRNP methyltransferase-like 2 (S. cerevisiae)
(HRMT1L2),”, “transcript variant 1, mRNA”, gi|38195088|ref|NM_001536.2|[38195088]; 246:
NM_001538, “Homo sapiens heat shock transcription factor 4 (HSF4), mRNA”,
gi|4557650|ref|NM_001538.1|[4557650]; 247: NM_001542, “Homo sapiens immunoglobulin
superfamily, member 3 (IGSF3), mRNA”, gi|4504626|ref|NM_001542.1|[4504626]; 248:
NM_001544, “Homo sapiens intercellular adhesion molecule 4, Landsteiner-Wiener blood
group”, “(ICAM4), transcript variant 1, mRNA”, gi|12545400|ref|NM_001544.2|[12545400];
249: NM_001545, “Homo sapiens immature colon carcinoma transcript 1 (ICT1), mRNA”,
gi|4557656|ref|NM_001545.1|[4557656]; 250: NM_001562, “Homo sapiens interleukin 18
(interferon-gamma-inducing factor) (IL18), mRNA”, gi|27502389|ref|NM_001562.2|[27502389];
251: NM_001567, “Homo sapiens inositol polyphosphate phosphatase-like 1 (INPPL1),
mRNA”, gi|4755141|ref|NM_001567.2|[4755141]; 252: NM_001569, “Homo sapiens
interleukin-1 receptor-associated kinase 1 (IRAK1), mRNA”,
gi|4755143|ref|NM_001569.2|[4755143]; 253: NM_001571, “Homo sapiens interferon
regulatory factor 3 (IRF3), mRNA”, gi|4504724|ref|NM_001571.1|[4504724]; 254: NM_001585,
“Homo sapiens chromosome 22 open reading frame 1 (C22orf1), mRNA”,
gi|31542268|ref|NM_001585.2|[31542268]; 255: NM_001610, “Homo sapiens acid phosphatase
2, lysosomal (ACP2), mRNA”, gi|4557009|ref|NM_001610.1|[4557009]; 256: NM_001615,
“Homo sapiens actin, gamma 2, smooth muscle, enteric (ACTG2), mRNA”,
gi|11038625|ref|NM_001615.2|[11038625]; 257: NM_001616, “Homo sapiens activin A
receptor, type II (ACVR2), mRNA”, gi|10862696|ref|NM_001616.2|[10862696]; 258:
NM_001618, Homo sapiens ADP-ribosyltransferase (NAD+; poly (ADP-ribose) polymerase),
“(ADPRT), mRNA”, gi|11496989|ref|NM_001618.2|[11496989]; 259: NM_001621, “Homo
sapiens aryl hydrocarbon receptor (AHR), mRNA”, gi|5016091|ref|NM_001621.2|[5016091];
260: NM_001622, “Homo sapiens alpha-2-HS-glycoprotein (AHSG), mRNA”,
gi|4502004|ref|NM_001622.1|[4502004]; 261: NM_001628, “Homo sapiens aldo-keto reductase
family 1, member B1 (aldose reductase)”, “(AKR1B1), mRNA”,
gi|24497579|ref|NM_001628.2|[24497579]; 262: NM_001629, “Homo sapiens arachidonate 5-
lipoxygenase-activating protein (ALOX5AP), mRNA”,
gi|15718674|ref|NM_001629.2|[15718674]; 263: NM_001637, “Homo sapiens acyloxyacyl
hydrolase (neutrophil) (AOAH), mRNA”, gi|4502114|ref|NM_001637.1|[4502114]; 264:
NM_001649, “Homo sapiens apical protein-like (Xenopus laevis) (APXL), mRNA”,
gi|18375508|ref|NM_001649.2|[18375508]; 265: NM_001654, “Homo sapiens v-raf murine
sarcoma 3611 viral oncogene homolog 1 (ARAF1), mRNA”,
gi|4502192|ref|NM_001654.1|[4502192]; 266: NM_001655, “Homo sapiens archain 1
(ARCN1), mRNA”, gi|21626463|ref|NM_001655.3|[21626463]; 267: NM_001662, “Homo
sapiens ADP-ribosylation factor 5 (ARF5), mRNA”, gi|6995999|ref|NM_001662.2|[6995999];
268: NM_001664, “Homo sapiens ras homolog gene family, member A (ARHA), mRNA”,
gi|10835048|ref|NM_001664.1|[10835048]; 269: NM_001666, “Homo sapiens Rho GTPase
activating protein 4 (ARHGAP4), mRNA”, gi|41327157|ref|NM_001666.2|[41327157]; 270:
NM_001671, “Homo sapiens asialoglycoprotein receptor 1 (ASGR1), mRNA”,
gi|18426870|ref|NM_001671.2|[18426870]; 271: NM_001673, “Homo sapiens asparagine
synthetase (ASNS), transcript variant 2, mRNA”, gi|19718771|ref|NM_001673.2|[19718771];
272: NM_001674, “Homo sapiens activating transcription factor 3 (ATF3), mRNA”,
gi|4502262|ref|NM_001674.1|[4502262]; 273: NM_001675, Homo sapiens activating
transcription factor 4 (tax-responsive enhancer element, “B67) (ATF4), transcript variant 1,
mRNA”, gi|33469975|ref|NM_001675.2|[33469975]; 274: NM_001678, “Homo sapiens
ATPase, Na+/K+ transporting, beta 2 polypeptide (ATP1B2), mRNA”,
gi|40254453|ref|NM_001678.2|[40254453]; 275: NM_001688, “Homo sapiens ATP synthase,
H+ transporting, mitochondrial F0 complex, subunit b,”, “isoform 1 (ATP5F1), mRNA”,
gi|21361564|ref|NM_001688.2|[21361564]; 276: NM_001702, “Homo sapiens brain-specific
angiogenesis inhibitor 1 (BAI1), mRNA”, gi|4502354|ref|NM_001702.1|[4502354]; 277:
NM_001722, “Homo sapiens polymerase (RNA) III (DNA directed) polypeptide D, 44 kDa
(POLR3D),”, mRNA, gi|4502436|ref|NM_001722.1|[4502436]; 278: NM_001724, “Homo
sapiens 2,3-bisphosphoglycerate mutase (BPGM), transcript variant 1, mRNA”,
gi|40353767|ref|NM_001724.3|[40353767]; 279: NM_001725, “Homo sapiens
bactericidal/permeability-increasing protein (BPI), mRNA”,
gi|4502446|ref|NM_001725.1|[4502446]; 280: NM_001739, “Homo sapiens carbonic anhydrase
VA, mitochondrial (CA5A), nuclear gene encoding”, “mitochondrial protein, mRNA”,
gi|4502520|ref|NM_001739.1|[4502520]; 281: NM_001744, “Homo sapiens
calcium/calmodulin-dependent protein kinase IV (CAMK4), mRNA”,
gi|27477118|ref|NM_001744.3|[27477118]; 282: NM_001747, “Homo sapiens capping protein
(actin filament), gelsolin-like (CAPG), mRNA”, gi|4502560|ref|NM_001747.1|[4502560]; 283:
NM_001760, “Homo sapiens cyclin D3 (CCND3), mRNA”,
gi|16950657|ref|NM_001760.2|[16950657]; 284: NM_001769, “Homo sapiens CD9 antigen
(p24) (CD9), mRNA”, gi|21237762|ref|NM_001769.2|[21237762]; 285: NM_001780, “Homo
sapiens CD63 antigen (melanoma 1 antigen) (CD63), mRNA”,
gi|34328936|ref|NM_001780.3|[34328936]; 286: NM_001796, “Homo sapiens cadherin 8, type
2 (CDH8), mRNA”, gi|16306538|ref|NM_001796.2|[16306538]; 287: NM_001799, “Homo
sapiens cyclin-dependent kinase 7 (MO15 homolog, Xenopus laevis,”, “cdk-activating kinase)
(CDK7), mRNA”, gi|16950659|ref|NM_001799.2|[16950659]; 288: NM_001806, “Homo
sapiens CCAAT/enhancer binding protein (C/EBP), gamma (CEBPG), mRNA”,
gi|34452718|ref|NM_001806.2|[34452718]; 289: NM_001810, “Homo sapiens centromere
protein B, 80 kDa (CENPB), mRNA”, gi|26105977|ref|NM_001810.4|[26105977]; 290:
NM_001821, “Homo sapiens choroideremia-like (Rab escort protein 2) (CHML), mRNA”,
gi|4502810|ref|NM_001821.1|[4502810]; 291: NM_001823, “Homo sapiens creatine kinase,
brain (CKB), mRNA”, gi|34335231|ref|NM_001823.3|[34335231]; 292: NM_001841, “Homo
sapiens cannabinoid receptor 2 (macrophage) (CNR2), mRNA”,
gi|4502928|ref|NM_001841.1|[4502928]; 293: NM_001842, “Homo sapiens ciliary neurotrophic
factor receptor (CNTFR), transcript variant 2,”, mRNA,
gi|22212916|ref|NM_001842.3|[22212916]; 294: NM_001843, “Homo sapiens contactin 1
(CNTN1), transcript variant 1, mRNA”, gi|28373116|ref|NM_001843.2|[28373116]; 295:
NM_001853, “Homo sapiens collagen, type IX, alpha 3 (COL9A3), mRNA”,
gi|17921994|ref|NM_001853.2|[17921994]; 296: NM_001855, “Homo sapiens collagen, type
XV, alpha 1 (COL15A1), mRNA”, gi|18641349|ref|NM_001855.2|[18641349]; 297:
NM_001856, “Homo sapiens collagen, type XVI, alpha 1 (COL16A1), mRNA”,
gi|18641351|ref|NM_001856.2|[18641351]; 298: NM_001859, “Homo sapiens solute carrier
family 31 (copper transporters), member 1 (SLC31A1),”, mRNA,
gi|40254457|ref|NM_001859.2|[40254457]; 299: NM_001863, “Homo sapiens cytochrome c
oxidase subunit VIb (COX6B), mRNA”, gi|17999530|ref|NM_001863.3|[17999530]; 300:
NM_001864, “Homo sapiens cytochrome c oxidase subunit VIIa polypeptide 1 (muscle)
(COX7A1),”, mRNA, gi|18105034|ref|NM_001864.2|[18105034]; 301: NM_001878, “Homo
sapiens cellular retinoic acid binding protein 2 (CRABP2), mRNA”,
gi|6382069|ref|NM_001878.2|[6382069]; 302: NM_001880, “Homo sapiens activating
transcription factor 2 (ATF2), mRNA”, gi|22538421|ref|NM_001880.2|[22538421]; 303:
NM_001885, “Homo sapiens crystallin, alpha B (CRYAB), mRNA”,
gi|4503056|ref|NM_001885.1|[4503056]; 304: NM_001887, “Homo sapiens crystallin, beta B1
(CRYBB1), mRNA”, gi|21536279|ref|NM_001887.3|[21536279]; 305: NM_001889, “Homo
sapiens crystallin, zeta (quinone reductase) (CRYZ), mRNA”,
gi|14251216|ref|NM_001889.2|[14251216]; 306: NM_001893, “Homo sapiens casein kinase 1,
delta (CSNK1D), transcript variant 1, mRNA”, gi|20544143|ref|NM_001893.3|[20544143]; 307:
NM_001895, “Homo sapiens casein kinase 2, alpha 1 polypeptide (CSNK2A1), transcript
variant”, “2, mRNA”, gi|29570794|ref|NM_001895.2|[29570794]; 308: NM_001905, “Homo
sapiens CTP synthase (CTPS), mRNA”, gi|4503132|ref|NM_001905.1|[4503132]; 309:
NM_001917, “Homo sapiens D-amino-acid oxidase (DAO), mRNA”,
gi|21536469|ref|NM_001917.3|[21536469]; 310: NM_001923, “Homo sapiens damage-specific
DNA binding protein 1, 127 kDa (DDB1), mRNA”, gi|13435358|ref|NM_001923.2|[13435358];
311: NM_001924, “Homo sapiens growth arrest and DNA-damage-inducible, alpha
(GADD45A), mRNA”, gi|9790904|ref|NM_001924.2|[9790904]; 312: NM_001928, “Homo
sapiens D component of complement (adipsin) (DF), mRNA”,
gi|42544238|ref|NM_001928.2|[42544238]; 313: NM_001932, “Homo sapiens membrane
protein, palmitoylated 3 (MAGUK p55 subfamily member 3)”, “(MPP3), mRNA”,
gi|21536463|ref|NM_001932.2|[21536463]; 314: NM_001933, Homo sapiens dihydrolipoamide
S-succinyltransferase (E2 component of, “2-oxo-glutarate complex) (DLST), mRNA”,
gi|32307170|ref|NM_001933.3|[32307170]; 315: NM_001944, “Homo sapiens desmoglein 3
(pemphigus vulgaris antigen) (DSG3), mRNA”, gi|4503404|ref|NM_001944.1|[4503404]; 316:
NM_001955, “Homo sapiens endothelin 1 (EDN1), mRNA”,
gi|21359861|ref|NM_001955.2|[21359861]; 317: NM_001958, “Homo sapiens eukaryotic
translation elongation factor 1 alpha 2 (EEF1A2), mRNA”,
gi|25453470|ref|NM_001958.2|[25453470]; 318: NM_001959, “Homo sapiens eukaryotic
translation elongation factor 1 beta 2 (EEF1B2),”, “transcript variant 1, mRNA”,
gi|16519564|ref|NM_001959.2|[16519564]; 319: NM_001962, “Homo sapiens ephrin-A5
(EFNA5), mRNA”, gi|4503486|ref|NM_001962.1|[4503486]; 320: NM_001967, “Homo sapiens
eukaryotic translation initiation factor 4A, isoform 2 (EIF4A2),”, mRNA,
gi|9945313|ref|NM_001967.2|[9945313]; 321: NM_001974, “Homo sapiens egf-like module
containing, mucin-like, hormone receptor-like 1”, “(EMR1), mRNA”,
gi|40807488|ref|NM_001974.3|[40807488]; 322: NM_001978, “Homo sapiens erythrocyte
membrane protein band 4.9 (dematin) (EPB49), mRNA”,
gi|4503580|ref|NM_001978.1|[4503580]; 323: NM_001985, “Homo sapiens electron-transfer-
flavoprotein, beta polypeptide (ETFB), mRNA”, gi|4503608|ref|NM_001985.1|[4503608]; 324:
NM_001989, “Homo sapiens eve, even-skipped homeo box homolog 1 (Drosophila) (EVX1),
mRNA”, gi|24497610|ref|NM_001989.2|[24497610]; 325: NM_001990, “Homo sapiens eyes
absent homolog 3 (Drosophila) (EYA3), transcript variant 1,”, mRNA,
gi|26667242|ref|NM_001990.2|[26667242]; 326: NM_001992, “Homo sapiens coagulation
factor II (thrombin) receptor (F2R), mRNA”, gi|6031164|ref|NM_001992.2|[6031164]; 327:
NM_002004, “Homo sapiens farnesyl diphosphate synthase (farnesyl pyrophosphate
synthetase,”, “dimethylallyltranstransferase, geranyltranstransferase) (FDPS), mRNA”,
gi|41281370|ref|NM_002004.2|[41281370]; 328: NM_002005, “Homo sapiens feline sarcoma
oncogene (FES), mRNA”, gi|13376997|ref|NM_002005.2|[13376997]; 329: NM_002010,
“Homo sapiens fibroblast growth factor 9 (glia-activating factor) (FGF9), mRNA”,
gi|4503706|ref|NM_002010.1|[4503706]; 330: NM_002012, “Homo sapiens fragile histidine
triad gene (FHIT), mRNA”, gi|4503718|ref|NM_002012.1|[4503718]; 331: NM_002020, “Homo
sapiens fms-related tyrosine kinase 4 (FLT4), transcript variant 2, mRNA”,
gi|4503752|ref|NM_002020.1|[4503752]; 332: NM_002022, “Homo sapiens flavin containing
monooxygenase 4 (FMO4), mRNA”, gi|4503758|ref|NM_002022.1|[4503758]; 333:
NM_002032, “Homo sapiens ferritin, heavy polypeptide 1 (FTH1), mRNA”,
gi|4503794|ref|NM_002032.1|[4503794]; 334: NM_002041, “Homo sapiens GA binding protein
transcription factor, beta subunit 2, 47 kDa”, “(GABPB2), transcript variant gamma-1, mRNA”,
gi|8051596|ref|NM_002041.2|[8051596]; 335: NM_002044, “Homo sapiens galactokinase 2
(GALK2), mRNA”, gi|4503896|ref|NM_002044.1|[4503896]; 336: NM_002047, “Homo sapiens
glycyl-tRNA synthetase (GARS), mRNA”, gi|6996009|ref|NM_002047.1|[6996009]; 337:
NM_002052, “Homo sapiens GATA binding protein 4 (GATA4), mRNA”,
gi|33188460|ref|NM_002052.2|[33188460]; 338: NM_002083, “Homo sapiens glutathione
peroxidase 2 (gastrointestinal) (GPX2), mRNA”, gi|32967606|ref|NM_002083.2|[32967606];
339: NM_002086, “Homo sapiens growth factor receptor-bound protein 2 (GRB2), mRNA”,
gi|34452726|ref|NM_002086.2|[34452726]; 340: NM_002093, “Homo sapiens glycogen
synthase kinase 3 beta (GSK3B), mRNA”, gi|21361339|ref|NM_002093.2|[21361339]; 341:
NM_002095, “Homo sapiens general transcription factor IIE, polypeptide 2, beta 34 kDa”,
“(GTF2E2), mRNA”, gi|34222295|ref|NM_002095.3|[34222295]; 342: NM_002110, “Homo
sapiens hemopoietic cell kinase (HCK), mRNA”, gi|30795228|ref|NM_002110.2|[30795228];
343: NM_002115, “Homo sapiens hexokinase 3 (white cell) (HK3), nuclear gene encoding”,
“mitochondrial protein, mRNA”, gi|4504394|ref|NM_002115.1|[4504394]; 344: NM_002137,
“Homo sapiens heterogeneous nuclear ribonucleoprotein A2/B1 (HNRPA2B1),”, “transcript
variant A2, mRNA”, gi|14043073|ref|NM_002137.2|[14043073]; 345: NM_002148, “Homo
sapiens homeo box D10 (HOXD10), mRNA”, gi|23510365|ref|NM_002148.2|[23510365]; 346:
NM_002151, “Homo sapiens hepsin (transmembrane protease, serine 1) (HPN), transcript
variant”, “2, mRNA”, gi|4504480|ref|NM_002151.1|[4504480]; 347: NM_002152, “Homo
sapiens histidine rich calcium binding protein (HRC), mRNA”,
gi|4504486|ref|NM_002152.1|[4504486]; 348: NM_002157, “Homo sapiens heat shock 10 kDa
protein 1 (chaperonin 10) (HSPE1), mRNA”, gi|4504522|ref|NM_002157.1|[45045221]; 349:
NM_002158, “Homo sapiens human T-cell leukemia virus enhancer factor (HTLF), mRNA”,
gi|40549453|ref|NM_002158.2|[40549453]; 350: NM_002162, “Homo sapiens intercellular
adhesion molecule 3 (ICAM3), mRNA”, gi|12545399|ref|NM_002162.2|[12545399]; 351:
NM_002193, “Homo sapiens inhibin, beta B (activin AB beta polypeptide) (INHBB), mRNA“;
gi|9257224|ref|NM_002193.1|[9257224]; 352: NM_002194, “Homo sapiens inositol
polyphosphate-1-phosphatase (INPP1), mRNA”, gi|4755138|ref|NM_002194.2|[4755138]; 353:
NM_002196, “Homo sapiens insulinoma-associated 1 (INSM1), mRNA”,
gi|4504712|ref|NM_002196.1|[4504712]; 354: NM_002198, “Homo sapiens interferon
regulatory factor 1 (IRF1), mRNA”, gi|4504720|ref|NM_002198.1|[4504720]; 355: NM_002199,
“Homo sapiens interferon regulatory factor 2 (IRF2), mRNA”,
gi|4755144|ref|NM_002199.2|[4755144]; 356: NM_002210, “Homo sapiens integrin, alpha V
(vitronectin receptor, alpha polypeptide, antigen”, “CD51) (ITGAV), mRNA”,
gi|40217844|ref|NM_002210.2|[40217844]; 357: NM_002212, “Homo sapiens integrin beta 4
binding protein (ITGB4BP), transcript variant 1,”, mRNA,
gi|31563381|ref|NM_002212.2|[31563381]; 358: NM_002217, “Homo sapiens pre-alpha
(globulin) inhibitor, H3 polypeptide (ITIH3), mRNA”,
gi|10092578|ref|NM_002217.1|[10092578]; 359: NM_002221, “Homo sapiens inositol 1,4,5-
trisphosphate 3-kinase B (ITPKB), mRNA”, gi|38569399|ref|NM_002221.2|[38569399]; 360:
NM_002229, “Homo sapiens jun B proto-oncogene (JUNB), mRNA”,
gi|4504808|ref|NM_002229.1|[4504808]; 361: NM_002231, “Homo sapiens kangai 1
(suppression of tumorigenicity 6, prostate; CD82 antigen”, “(R2 leukocyte antigen, antigen
detected by monoclonal and antibody IA4)) (KAI1),”, mRNA,
gi|13259537|ref|NM_002231.2|[13259537]; 362: NM_002232, “Homo sapiens potassium
voltage-gated channel, shaker-related subfamily, member 3”, “(KCNA3), mRNA”,
gi|25952081|ref|NM_002232.2|[25952081]; 363: NM_002238, “Homo sapiens potassium
voltage-gated channel, subfamily H (eag-related), member”, “1 (KCNH1), transcript variant 2,
mRNA”, gi|27436999|ref|NM_002238.2|[27436999]; 364: NM_002241, “Homo sapiens
potassium inwardly-rectifying channel, subfamily J, member 10”, “(KCNJ10), mRNA”,
gi|25121965|ref|NM_002241.2|[25121965]; 365: NM_002248, “Homo sapiens potassium
intermediate/small conductance calcium-activated channel,”, “subfamily N, member 1 (KCNN1),
mRNA”, gi|25777642|ref|NM_002248.3|[25777642]; 366: NM_002252, “Homo sapiens
potassium voltage-gated channel, delayed-rectifier, subfamily S,”, “member 3 (KCNS3),
mRNA”, gi|25952107|ref|NM_002252.3|[25952107]; 367: NM_002257, “Homo sapiens
kallikrein 1, renal/pancreas/salivary (KLK1), mRNA”,
gi|22027643|ref|NM_002257.2|[22027643]; 368: NM_002268, “Homo sapiens karyopherin
alpha 4 (importin alpha 3) (KPNA4), mRNA”, gi|27477125|ref|NM_002268.3|[27477125]; 369:
NM_002277, “Homo sapiens keratin, hair, acidic, 1 (KRTHA1), mRNA”,
gi|14917114|ref|NM_002277.2|[14917114]; 370: NM_002280, “Homo sapiens keratin, hair,
acidic, 5 (KRTHA5), mRNA”, gi|15431313|ref|NM_002280.3|[15431313]; 371: NM_002283,
“Homo sapiens keratin, hair, basic, 5 (KRTHB5), mRNA”,
gi|15431324|ref|NM_002283.2|[15431324]; 372: NM_002286, “Homo sapiens lymphocyte-
activation gene 3 (LAG3), mRNA”, gi|15718681|ref|NM_002286.4|[15718681]; 373:
NM_002298, “Homo sapiens lymphocyte cytosolic protein 1 (L-plastin) (LCP1), mRNA”,
gi|7382490|ref|NM_002298.2|[7382490]; 374: NM_002305, “Homo sapiens lectin, galactoside-
binding, soluble, 1 (galectin 1) (LGALS1), mRNA”, gi|6006015|ref|NM_002305.2|[6006015];
375: NM_002309, Homo sapiens leukemia inhibitory factor (cholinergic differentiation factor),
“(LIF), mRNA”, gi|6006018|ref|NM_002309.2|[6006018]; 376: NM_002312, “Homo sapiens
ligase IV, DNA, ATP-dependent (LIG4), mRNA”, gi|23199992|ref|NM_002312.2|[23199992];
377: NM_002316, “Homo sapiens LIM homeobox transcription factor 1, beta (LMX1B),
mRNA”, gi|4505006|ref|NM_002316.1|[4505006]; 378: NM_002335, “Homo sapiens low
density lipoprotein receptor-related protein 5 (LRP5), mRNA”,
gi|4505018|ref|NM_002335.1|[4505018]; 379: NM_002339, “Homo sapiens lymphocyte-
specific protein 1 (LSP1), mRNA”, gi|10880978|ref|NM_002339.1|[10880978]; 380:
NM_002342, “Homo sapiens lymphotoxin beta receptor (TNFR superfamily, member 3)
(LTBR), mRNA”, gi|4505038|ref|NM_002342.1|[4505038]; 381: NM_002347, “Homo sapiens
lymphocyte antigen 6 complex, locus H (LY6H), mRNA”,
gi|4505050|ref|NM_002347.1|[4505050]; 382: NM_002357, “Homo sapiens MAX dimerization
protein 1 (MAD), mRNA”, gi|4505068|ref|NM_002357.1|[4505068]; 383: NM_002372, “Homo
sapiens mannosidase, alpha, class 2A, member 1 (MAN2A1), mRNA”,
gi|4758697|ref|NM_002372.1|[4758697]; 384: NM_002378, “Homo sapiens megakaryocyte-
associated tyrosine kinase (MATK), transcript variant”, “2, mRNA”,
gi|21450841|ref|NM_002378.2|[21450841]; 385: NM_002381, “Homo sapiens matrilin 3
(MATN3), mRNA”, gi|13518040|ref|NM_002381.2|[13518040]; 386: NM_002386, Homo
sapiens melanocortin 1 receptor (alpha melanocyte stimulating hormone, “receptor) (MC1R),
mRNA”, gi|27477128|ref|NM_002386.2|[27477128]; 387: NM_002388, “Homo sapiens MCM3
minichromosome maintenance deficient 3 (S. cerevisiae) (MCM3),”, mRNA,
gi|33356548|ref|NM_002388.3|[33356548]; 388: NM_002390, “Homo sapiens a disintegrin and
metalloproteinase domain 11 (ADAM11), transcript”, “variant 1, mRNA”,
gi|4585709|ref|NM_002390.2|[4585709]; 389: NM_002391, “Homo sapiens midkine (neurite
growth-promoting factor 2) (MDK), mRNA”, gi|24475622|ref|NM_002391.2|[24475622]; 390:
NM_002393, “Homo sapiens Mdm4, transformed 3T3 cell double minute 4, p53 binding
protein”, “(mouse) (MDM4), mRNA”, gi|4505138|ref|NM_002393.1|[4505138]; 391:
NM_002398, “Homo sapiens Meis1, myeloid ecotropic viral integration site 1 homolog
(mouse)”, “(MEIS1), mRNA”, gi|4505150|ref|NM_002398.1|[4505150]; 392: NM_002399,
“Homo sapiens Meis1, myeloid ecotropic viral integration site 1 homolog 2 (mouse)”, “(MEIS2),
transcript variant f, mRNA”, gi|27502374|ref|NM_002399.2|[27502374]; 393: NM_002401,,
ref|NM_002401.3|[42794764]; 394: NM_002406, “Homo sapiens mannosyl (alpha-1,3-)-
glycoprotein”, “beta-1,2-N-acetylglucosaminyltransferase (MGAT1), mRNA”,
gi|6031182|ref|NM_002406.2|[6031182]; 395: NM_002412, “Homo sapiens O-6-
methylguanine-DNA methyltransferase (MGMT), mRNA”,
gi|4505176|ref|NM_002412.1|[4505176]; 396: NM_002419, “Homo sapiens mitogen-activated
protein kinase kinase kinase 11 (MAP3K11), mRNA”,
gi|21735553|ref|NM_002419.2|[21735553]; 397: NM_002427, “Homo sapiens matrix
metalloproteinase 13 (collagenase 3) (MMP13), mRNA”,
gi|13027796|ref|NM_002427.2|[13027796]; 398: NM_002428, “Homo sapiens matrix
metalloproteinase 15 (membrane-inserted) (MMP15), mRNA”,
gi|4505210|ref|NM_002428.1|[4505210]; 399: NM_002434, “Homo sapiens N-methylpurine-
DNA glycosylase (MPG), mRNA”, gi|4505232|ref|NM_002434.1|[4505232]; 400: NM_002437,
“Homo sapiens MpV17 transgene, murine homolog, glomerulosclerosis (MPV17), mRNA”,
gi|37059781|ref|NM_002437.3|[37059781]; 401: NM_002446, “Homo sapiens mitogen-
activated protein kinase kinase kinase 10 (MAP3K10), mRNA”,
gi|21735549|ref|NM_002446.2|[21735549]; 402: NM_002447, Homo sapiens macrophage
stimulating 1 receptor (c-met-related tyrosine kinase), “(MST1R), mRNA”,
gi|4505264|ref|NM_002447.1|[4505264]; 403: NM_002452, Homo sapiens nudix (nucleoside
diphosphate linked moiety X)-type motif 1, “(NUDT1), transcript variant 1, mRNA”,
gi|40288273|ref|NM_002452.3|[40288273]; 404: NM_002453, “Homo sapiens mitochondrial
translational initiation factor 2 (MTIF2), nuclear”, “gene encoding mitochondrial protein,
mRNA”, gi|4505276|ref|NM_002453.1|[4505276]; 405: NM_002461, “Homo sapiens
mevalonate (diphospho) decarboxylase (MVD), mRNA”,
gi|4505288|ref|NM_002461.1|[4505288]; 406: NM_002470, “Homo sapiens myosin, heavy
polypeptide 3, skeletal muscle, embryonic (MYH3),”, mRNA,
gi|11342671|ref|NM_002470.1|[11342671]; 407: NM_002471, “Homo sapiens myosin, heavy
polypeptide 6, cardiac muscle, alpha (cardiomyopathy,”, “hypertrophic 1) (MYH6), mRNA”,
gi|27764860|ref|NM_002471.1|[27764860]; 408: NM_002475, “Homo sapiens myosin light
chain 1 slow a (MLC1SA), mRNA”, gi|17986280|ref|NM_002475.2|[17986280]; 409:
NM_002487, “Homo sapiens necdin homolog (mouse) (NDN), mRNA”,
gi|10800414|ref|NM_002487.2|[10800414]; 410: NM_002492, “Homo sapiens NADH
dehydrogenase (ubiquinone) 1 beta subcomplex, 5, 16 kDa”, “(NDUFB5), nuclear gene encoding
mitochondrial protein, mRNA”, gi|33519467|ref|NM_002492.2|[33519467]; 411: NM_002500,
“Homo sapiens neurogenic differentiation 1 (NEUROD1), mRNA”,
gi|4505376|ref|NM_002500.1|[4505376]; 412: NM_002506, “Homo sapiens nerve growth
factor, beta polypeptide (NGFB), mRNA”, gi|4505390|ref|NM_002506.1|[4505390]; 413:
NM_002513, “Homo sapiens non-metastatic cells 3, protein expressed in (NME3), mRNA”,
gi|37693992|ref|NM_002513.2|[37693992]; 414: NM_002522, “Homo sapiens neuronal
pentraxin I (NPTX1), mRNA”, gi|4505442|ref|NM_002522.1|[4505442]; 415: NM_002525,
“Homo sapiens nardilysin (N-arginine dibasic convertase) (NRD1), mRNA”,
gi|4505452|ref|NM_002525.1|[4505452]; 416: NM_002528, “Homo sapiens nth endonuclease
III-like 1 (E. coli) (NTHL1), mRNA”, gi|38455392|ref|NM_002528.4|[38455392]; 417:
NM_002529, “Homo sapiens neurotrophic tyrosine kinase, receptor, type 1 (NTRK1), mRNA”,
gi|4585711|ref|NM_002529.2|[4585711]; 418: NM_002531, “Homo sapiens neurotensin
receptor 1 (high affinity) (NTSR1), mRNA”, gi|4505476|ref|NM_002531.1|[4505476]; 419:
NM_002555, “Homo sapiens solute carrier family 22 (organic cation transporter), member 18”,
“(SLC22A18), transcript variant 1, mRNA”, gi|34734074|ref|NM_002555.3|[34734074]; 420:
NM_002559, “Homo sapiens purinergic receptor P2X, ligand-gated ion channel, 3 (P2RX3),
mRNA”, gi|28416924|ref|NM_002559.2|[28416924]; 421: NM_002560, “Homo sapiens
purinergic receptor P2X, ligand-gated ion channel, 4 (P2RX4),”, “transcript variant 1, mRNA”,
gi|28416926|ref|NM_002560.2|[28416926]; 422: NM_002562, “Homo sapiens purinergic
receptor P2X, ligand-gated ion channel, 7 (P2RX7),”, “transcript variant 1, mRNA”,
gi|34335273|ref|NM_002562.4|[34335273]; 423: NM_002563, “Homo sapiens purinergic
receptor P2Y, G-protein coupled, 1 (P2RY1), mRNA”,
gi|28872741|ref|NM_002563.2|[28872741]; 424: NM_002566, “Homo sapiens purinergic
receptor P2Y, G-protein coupled, 11 (P2RY11), mRNA”,
gi|29029602|ref|NM_002566.3|[29029602]; 425: NM_002568, “Homo sapiens poly(A) binding
protein, cytoplasmic 1 (PABPC1), mRNA”, gi|4505574|ref|NM_002568.1|[4505574]; 426:
NM_002569, “Homo sapiens furin (paired basic amino acid cleaving enzyme) (FURIN),
mRNA”, gi|20336193|ref|NM_002569.2|[20336193]; 427: NM_002572, “Homo sapiens
platelet-activating factor acetylhydrolase, isoform Ib, beta”, “subunit 30 kDa (PAFAH1B2),
mRNA”, gi|4505584|ref|NM_002572.1|[4505584]; 428: NM_002576,,
ref|NM_002576.3|[42794768]; 429: NM_002582, “Homo sapiens poly(A)-specific ribonuclease
(deadenylation nuclease) (PARN), mRNA”, gi|4505610|ref|NM_002582.1|[4505610]; 430:
NM_002584, “Homo sapiens paired box gene 7 (PAX7), transcript variant 1, mRNA”,
gi|4505618|ref|NM_002584.1|[4505618]; 431: NM_002590, “Homo sapiens protocadherin 8
(PCDH8), transcript variant 1, mRNA”, gi|6631101|ref|NM_002590.2|[6631101]; 432:
NM_002591, “Homo sapiens phosphoenolpyruvate carboxykinase 1 (soluble) (PCK1), mRNA”,
gi|32483400|ref|NM_002591.2|[32483400]; 433: NM_002599, “Homo sapiens
phosphodiesterase 2A, cGMP-stimulated (PDE2A), mRNA”,
gi|4505656|ref|NM_002599.1|[4505656]; 434: NM_002615, “Homo sapiens serine (or cysteine)
proteinase inhibitor, clade F (alpha-2”, “antiplasmin, pigment epithelium derived factor),
member 1 (SERPINF1), mRNA”, gi|39725933|ref|NM_002615.3|[39725933]; 435: NM_002618,
“Homo sapiens peroxisome biogenesis factor 13 (PEX13), mRNA”,
gi|4505722|ref|NM_002618.1|[4505722]; 436: NM_002620, “Homo sapiens platelet factor 4
variant 1 (PF4V1), mRNA”, gi|4505734|ref|NM_002620.1|[4505734]; 437: NM_002628,
“Homo sapiens profilin 2 (PFN2), transcript variant 2, mRNA”,
gi|16753216|ref|NM_002628.2|[16753216]; 438: NM_002630, “Homo sapiens progastricsin
(pepsinogen C) (PGC), mRNA”, gi|4505756|ref|NM_002630.1|[4505756]; 439: NM_002635,
Homo sapiens solute carrier family 25 (mitochondrial carrier; phosphate, “carrier), member 3
(SLC25A3), nuclear gene encoding mitochondrial protein,”, “transcript variant 1b, mRNA”,
gi|4505774|ref|NM_002635.1|[4505774]; 440: NM_002639, “Homo sapiens serine (or cysteine)
proteinase inhibitor, clade B (ovalbumin),”, “member 5 (SERPINB5), mRNA”,
gi|4505788|ref|NM_002639.1|[4505788]; 441: NM_002640, “Homo sapiens serine (or cysteine)
proteinase inhibitor, clade B (ovalbumin),”, “member 8 (SERPINB8), transcript variant 1,
mRNA”, gi|38504672|ref|NM_002640.3|[38504672]; 442: NM_002641, “Homo sapiens
phosphatidylinositol glycan, class A (paroxysmal nocturnal”, “hemoglobinuria) (PIGA),
transcript variant 1, mRNA”, gi|11863129|ref|NM_002641.1|[11863129]; 443: NM_002648,
“Homo sapiens pim-1 oncogene (PIM1), mRNA”, gi|31543400|ref|NM_002648.2|[31543400];
444: NM_002654, “Homo sapiens pyruvate kinase, muscle (PKM2), transcript variant 1,
mRNA”, gi|33286417|ref|NM_002654.3|[33286417]; 445: NM_002655, “Homo sapiens
pleiomorphic adenoma gene 1 (PLAG1), mRNA”, gi|4505854|ref|NM_002655.1|[4505854]; 446:
NM_002676, “Homo sapiens phosphomannomutase 1 (PMM1), mRNA”,
gi|4505904|ref|NM_002676.1|[4505904]; 447: NM_002692, “Homo sapiens polymerase (DNA
directed), epsilon 2 (p59 subunit) (POLE2), mRNA”,
gi|32189368|ref|NM_002692.2|[32189368]; 448: NM_002697, “Homo sapiens POU domain,
class 2, transcription factor 1 (POU2F1), mRNA”, gi|42476163|ref|NM_002697.2|[42476163];
449: NM_002707, “Homo sapiens protein phosphatase 1G (formerly 2C), magnesium-
dependent, gamma”, “isoform (PPM1G), transcript variant 2, mRNA”,
gi|29826283|ref|NM_002707.3|[29826283]; 450: NM_002708, “Homo sapiens protein
phosphatase 1, catalytic subunit, alpha isoform (PPP1CA),”, mRNA,
gi|31543430|ref|NM_002708.2|[31543430]; 451: NM_002715, “Homo sapiens protein
phosphatase 2 (formerly 2A), catalytic subunit, alpha”, “isoform (PPP2CA), mRNA”,
gi|4506016|ref|NM_002715.1|[4506016]; 452: NM_002728, “Homo sapiens proteoglycan 2,
bone marrow (natural killer cell activator,”, “eosinophil granule major basic protein) (PRG2),
mRNA”, gi|32261294|ref|NM_002728.3|[32261294]; 453: NM_002739, “Homo sapiens protein
kinase C, gamma (PRKCG), mRNA”, gi|31377808|ref|NM_002739.2|[31377808]; 454:
NM_002763, “Homo sapiens prospero-related homeobox 1 (PROX1), mRNA”,
gi|34147628|ref|NM_002763.3|[34147628]; 455: NM_002766, Homo sapiens phosphoribosyl
pyrophosphate synthetase-associated protein 1, “(PRPSAP1), mRNA”,
gi|4506130|ref|NM_002766.1|[4506130]; 456: NM_002768, “Homo sapiens procollagen (type
III) N-endopeptidase (PCOLN3), mRNA”, gi|4506138|ref|NM_002768.1|[4506138]; 457:
NM_002774, “Homo sapiens kallikrein 6 (neurosin, zyme) (KLK6), mRNA”,
gi|21327702|ref|NM_002774.2|[21327702]; 458: NM_002779, “Homo sapiens pleckstrin and
Sec7 domain protein (PSD), mRNA”, gi|28626518|ref|NM_002779.2|[28626518]; 459:
NM_002789, “Homo sapiens proteasome (prosome, macropain) subunit, alpha type, 4
(PSMA4),”, mRNA, gi|23110940|ref|NM_002789.3|[23110940]; 460: NM_002790, “Homo
sapiens proteasome (prosome, macropain) subunit, alpha type, 5 (PSMA5),”, mRNA,
gi|23110941|ref|NM_002790.2|[23110941]; 461: NM_002791, “Homo sapiens proteasome
(prosome, macropain) subunit, alpha type, 6 (PSMA6),”, mRNA,
gi|23110943|ref|NM_002791.1|[23110943]; 462: NM_002795, “Homo sapiens proteasome
(prosome, macropain) subunit, beta type, 3 (PSMB3), mRNA”,
gi|22538464|ref|NM_002795.2|[22538464]; 463: NM_002799, “Homo sapiens proteasome
(prosome, macropain) subunit, beta type, 7 (PSMB7), mRNA”,
gi|23110926|ref|NM_002799.2|[23110926]; 464: NM_002800, “Homo sapiens proteasome
(prosome, macropain) subunit, beta type, 9 (large”, “multifunctional protease 2) (PSMB9),
transcript variant 1, mRNA”, gi|23110930|ref|NM_002800.3|[23110930]; 465: NM_002802,
“Homo sapiens proteasome (prosome, macropain) 26S subunit, ATPase, 1 (PSMC1),”, mRNA,
gi|24430150|ref|NM_002802.2|[24430150]; 466: NM_002805, “Homo sapiens proteasome
(prosome, macropain) 26S subunit, ATPase, 5 (PSMC5),”, mRNA,
gi|24497434|ref|NM_002805.4|[24497434]; 467: NM_002818, “Homo sapiens proteasome
(prosome, macropain) activator subunit 2 (PA28 beta)”, “(PSME2), mRNA”,
gi|30410791|ref|NM_002818.2|[30410791]; 468: NM_002821, “Homo sapiens PTK7 protein
tyrosine kinase 7 (PTK7), transcript variant PTK7-1,”, mRNA,
gi|27886610|ref|NM_002821.3|[27886610]; 469: NM_002826, “Homo sapiens quiescin Q6
(QSCN6), mRNA”, gi|13325074|ref|NM_002826.2|[13325074]; 470: NM_002831, “Homo
sapiens protein tyrosine phosphatase, non-receptor type 6 (PTPN6),”, “transcript variant 1,
mRNA”, gi|34328900|ref|NM_002831.3|[34328900]; 471: NM_002832, “Homo sapiens protein
tyrosine phosphatase, non-receptor type 7 (PTPN7),”, “transcript variant 1, mRNA”,
gi|18375657|ref|NM_002832.2|[18375657]; 472: NM_002833, “Homo sapiens protein tyrosine
phosphatase, non-receptor type 9 (PTPN9), mRNA”, gi|18375663|ref|NM_002833.2|[18375663];
473: NM_002837, “Homo sapiens protein tyrosine phosphatase, receptor type, B (PTPRB),
mRNA”, gi|18491009|ref|NM_002837.2|[18491009]; 474: NM_002841, “Homo sapiens protein
tyrosine phosphatase, receptor type, G (PTPRG), mRNA”,
gi|18860897|ref|NM_002841.2|[18860897]; 475: NM_002845, “Homo sapiens protein tyrosine
phosphatase, receptor type, M (PTPRM), mRNA”, gi|18860903|ref|NM_002845.2|[18860903];
476: NM_002851, “Homo sapiens protein tyrosine phosphatase, receptor-type, Z polypeptide
1”, “(PTPRZ1), mRNA”, gi|4506328|ref|NM_002851.1|[4506328]; 477: NM_002854, “Homo
sapiens parvalbumin (PVALB), mRNA”, gi|4506334|ref|NM_002854.1|[4506334]; 478:
NM_002856, Homo sapiens poliovirus receptor-related 2 (herpesvirus entry mediator B),
“(PVRL2), mRNA”, gi|5360209|ref|NM_002856.1|[5360209]; 479: NM_002860, Homo sapiens
pyrroline-5-carboxylate synthetase (glutamate gamma-semialdehyde, “synthetase) (PYCS),
mRNA”, gi|21361367|ref|NM_002860.2|[21361367]; 480: NM_002863, “Homo sapiens
phosphorylase, glycogen; liver (Hers disease, glycogen storage”, “disease type VI) (PYGL),
mRNA”, gi|42476165|ref|NM_002863.2|[42476165]; 481: NM_002868, “Homo sapiens
RAB5B, member RAS oncogene family (RAB5B), mRNA”,
gi|33943097|ref|NM_002868.2|[33943097]; 482: NM_002887, “Homo sapiens arginyl-tRNA
synthetase (RARS), mRNA”, gi|40068503|ref|NM_002887.3|[40068503]; 483: NM_002891,
Homo sapiens Ras protein-specific guanine nucleotide-releasing factor 1, “(RASGRF1),
transcript variant 1, mRNA”, gi|24797098|ref|NM_002891.3|[24797098]; 484: NM_002892,
“Homo sapiens AT rich interactive domain 4A (RBP1-like) (ARID4A), transcript”, “variant 1,
mRNA”, gi|13259496|ref|NM_002892.2|[13259496]; 485: NM_002900, “Homo sapiens retinol
binding protein 3, interstitial (RBP3), mRNA”, gi|4506452|ref|NM_002900.1|[4506452]; 486:
NM_002901, “Homo sapiens reticulocalbin 1, EF-hand calcium binding domain (RCN1),
mRNA”, gi|4506454|ref|NM_002901.1|[4506454]; 487: NM_002904, “Homo sapiens RD RNA
binding protein (RDBP), mRNA”, gi|20631983|ref|NM_002904.4|[20631983]; 488: NM_002912,
“Homo sapiens REV3-like, catalytic subunit of DNA polymerase zeta (yeast)”, “(REV3L),
mRNA”, gi|4506482|ref|NM_002912.1|[4506482]; 489: NM_002916, “Homo sapiens
replication factor C (activator 1) 4, 37 kDa (RFC4), transcript”, “variant 1, mRNA”,
gi|31881681|ref|NM_002916.3|[31881681]; 490: NM_002919, “Homo sapiens regulatory factor
X, 3 (influences HLA class II expression) (RFX3),”, “transcript variant 1, mRNA”,
gi|19743882|ref|NM_002919.2|[19743882]; 491: NM_002921,“Homo sapiens retinal G protein
coupled receptor (RGR), xmRNA”, gi|21361328|ref|NM_002921.2|[21361328]; 492: NM_002923,
“Homo sapiens regulator of G-protein signalling 2, 24 kDa (RGS2), mRNA”,
gi|4506516|ref|NM_002923.1|[4506516]; 493: NM_002930, “Homo sapiens Ras-like without
CAAX 2 (RIT2), mRNA”, gi|4506532|ref|NM_002930.1|[4506532]; 494: NM_002938, “Homo
sapiens ring finger protein 4 (RNF4), mRNA”, gi|34305289|ref|NM_002938.2|[34305289]; 495:
NM_002941, “Homo sapiens roundabout, axon guidance receptor, homolog 1 (Drosophila)
(ROBO1),”, “transcript variant 1, mRNA”, gi|19743804|ref|NM_002941.2|[19743804]; 496:
NM_002946, “Homo sapiens replication protein A2, 32 kDa (RPA2), mRNA”,
gi|34147622|ref|NM_002946.3|[34147622]; 497: NM_002954, “Homo sapiens ribosomal
protein S27a (RPS27A), mRNA”, gi|27436941|ref|NM_002954.3|[27436941]; 498: NM_002965,
“Homo sapiens S100 calcium binding protein A9 (calgranulin B) (S100A9), mRNA”,
gi|9845520|ref|NM_002965.2|[9845520]; 499: NM_002966, “Homo sapiens S100 calcium
binding protein A10 (annexin II ligand, calpactin I,”, “light polypeptide (p11)) (S100A10),
mRNA”, gi|4506760|ref|NM_002966.1|[4506760]; 500: NM_002968, “Homo sapiens sal-like 1
(Drosophila) (SALL1), mRNA”, gi|6997248|ref|NM_002968.1|[6997248]; 501: NM_002971,
Homo sapiens special AT-rich sequence binding protein 1 (binds to nuclear, “matrix/scaffold-
associating DNA's) (SATB1), mRNA”, gi|33356175|ref|NM_002971.2|[33356175]; 502:
NM_002973, “Homo sapiens spinocerebellar ataxia 2 (olivopontocerebellar ataxia 2,
autosomal”, “dominant, ataxin 2) (SCA2), mRNA”, gi|4506794|ref|NM_002973.1|[4506794];
503: NM_002987, “Homo sapiens chemokine (C—C motif) ligand 17 (CCL17), mRNA”,
gi|22538801|ref|NM_002987.2|[22538801]; 504: NM_003002, “Homo sapiens succinate
dehydrogenase complex, subunit D, integral membrane”, “protein (SDHD), nuclear gene
encoding mitochondrial protein, mRNA”, gi|4506864|ref|NM_003002.1|[4506864]; 505:
NM_003025, “Homo sapiens SH3-domain GRB2-like 1 (SH3GL1), mRNA”,
gi|42476326|ref|NM_003025.2|[42476326]; 506: NM_003028, “Homo sapiens SUB (Src
homology 2 domain containing) adaptor protein B (SUB),”, mRNA,
gi|4506934|ref|NM_003028.1|[4506934]; 507: NM_003034, Homo sapiens sialyltransferase 8A
(alpha-N-acetylneuraminate:, “alpha-2,8-sialyltransferase, GD3 synthase) (SIAT8A), mRNA”,
gi|28373095|ref|NM_003034.2|[28373095]; 508: NM_003035, “Homo sapiens TAL1 (SCL)
interrupting locus (SIL), mRNA”, gi|4506958|ref|NM_003035.1|[4506958]; 509: NM_003040,
“Homo sapiens solute carrier family 4, anion exchanger, member 2 (erythrocyte”, “membrane
protein band 3-like 1) (SLC4A2), mRNA”, gi|21361550|ref|NM_003040.2|[21361550]; 510:
NM_003042, “Homo sapiens solute carrier family 6 (neurotransmitter transporter, GABA),”,
“member 1 (SLC6A1), mRNA”, gi|40254466|ref|NM_003042.2|[40254466]; 511: NM_003054,
“Homo sapiens solute carrier family 18 (vesicular monoamine), member 2 (SLC18A2),”,
mRNA, gi|42476324|ref|NM_003054.2|[42476324]; 512: NM_003055, “Homo sapiens solute
carrier family 18 (vesicular acetylcholine), member 3”, “(SLC18A3), mRNA”,
gi|4506990|ref|NM_003055.1|[4506990]; 513: NM_003058, “Homo sapiens solute carrier
family 22 (organic cation transporter), member 2”, “(SLC22A2), transcript variant 1, mRNA”,
gi|23510411|ref|NM_003058.2|[23510411]; 514: NM_003068, “Homo sapiens snail homolog 2
(Drosophila) (SNAI2), mRNA”, gi|24497625|ref|NM_003068.3|[24497625]; 515: NM_003077,
“Homo sapiens SWI/SNF related, matrix associated, actin dependent regulator of”, “chromatin,
subfamily d, member 2 (SMARCD2), mRNA”, gi|21264350|ref|NM_003077.2|[21264350]; 516:
NM_003092, “Homo sapiens small nuclear ribonucleoprotein polypeptide B″ (SNRPB2),”,
“transcript variant 1, mRNA”, gi|38149917|ref|NM_003092.3|[38149917]; 517: NM_003093,
“Homo sapiens small nuclear ribonucleoprotein polypeptide C (SNRPC), mRNA”,
gi|4507126|ref|NM_003093.1|[4507126]; 518: NM_003096, “Homo sapiens small nuclear
ribonucleoprotein polypeptide G (SNRPG), mRNA”, gi|21359839|ref|NM_003096.2|[21359839];
519: NM_003115, “Homo sapiens UDP-N-acteylglucosamine pyrophosphorylase 1 (UAP1),
mRNA”, gi|34147515|ref|NM_003115.3|[34147515]; 520: NM_003132, “Homo sapiens
spermidine synthase (SRM), mRNA”, gi|4507208|ref|NM_003132.1|[4507208]; 521:
NM_003134, Homo sapiens signal recognition particle 14 kDa (homologous Alu RNA binding,
“protein) (SRP14), mRNA”, gi|31543652|ref|NM_003134.2|[31543652]; 522: NM_003135,
“Homo sapiens signal recognition particle 19 kDa (SRP19), mRNA”,
gi|4507212|ref|NM_003135.1|[4507212]; 523: NM_003140, “Homo sapiens sex determining
region Y (SRY), mRNA”, gi|4507224|ref|NM_003140.1|[4507224]; 524: NM_003141, “Homo
sapiens Sjogren syndrome antigen A1 (52 kDa, ribonucleoprotein autoantigen”, “SS-A/Ro)
(SSA1), mRNA”, gi|15208659|ref|NM_003141.2|[15208659]; 525: NM_003149, “Homo
sapiens src homology three (SH3) and cysteine rich domain (STAC), mRNA”,
gi|4507246|ref|NM_003149.1|[4507246]; 526: NM_003150, Homo sapiens signal transducer
and activator of transcription 3 (acute-phase, “response factor) (STAT3), transcript variant 2,
mRNA”, gi|21618337|ref|NM_003150.2|[21618337]; 527: NM_003156, “Homo sapiens stromal
interaction molecule 1 (STIM1), mRNA”, gi|21070996|ref|NM_003156.2|[21070996]; 528:
NM_003159, “Homo sapiens cyclin-dependent kinase-like 5 (CDKL5), mRNA”,
gi|4507280|ref|NM_003159.1|[4507280]; 529: NM_003162, “Homo sapiens striatin, calmodulin
binding protein (STRN), mRNA”, gi|4507282|ref|NM_003162.1|[4507282]; 530: NM_003165,
“Homo sapiens syntaxin binding protein 1 (STXBP1), mRNA”,
gi|4507296|ref|NM_003165.1|[4507296]; 531: NM_003181, “Homo sapiens T, brachyury
homolog (mouse) (T), mRNA”, gi|19743811|ref|NM_003181.2|[19743811]; 532: NM_003184,
“Homo sapiens TAF2 RNA polymerase II, TATA box binding protein (TBP)-associated”,
“factor, 150 kDa (TAF2), mRNA”, gi|20357590|ref|NM_003184.2|[20357590]; 533: NM_003186,
“Homo sapiens transgelin (TAGLN), mRNA”, gi|12621918|ref|NM_003186.2|[12621918]; 534:
NM_003192, “Homo sapiens tubulin-specific chaperone c (TBCC), mRNA”,
gi|4507372|ref|NM_003192.1|[4507372]; 535: NM_003194, “Homo sapiens TATA box binding
protein (TBP), mRNA”, gi|20544178|ref|NM_003194.2|[20544178]; 536: NM_003216, “Homo
sapiens thyrotrophic embryonic factor (TEF), mRNA”,
gi|34486096|ref|NM_003216.2|[34486096]; 537: NM_003223, Homo sapiens transcription
factor AP-4 (activating enhancer binding protein 4), “(TFAP4), mRNA”,
gi|4507446|ref|NM_003223.1|[4507446]; 538: NM_003239, “Homo sapiens transfonning
growth factor, beta 3 (TGFB3), mRNA”, gi|4507464|ref|NM_003239.1|[4507464]; 539:
NM_003245, “Homo sapiens transglutaminase 3 (E polypeptide,”, “protein-glutamine-gamma-
glutamyltransferase) (TGM3), mRNA”, gi|39777600|ref|NM_003245.2|[39777600]; 540:
NM_003256, “Homo sapiens tissue inhibitor of metalloproteinase 4 (TIMP4), mRNA”,
gi|4507514|ref|NM_003256.1|[4507514]; 541: NM_003259, “Homo sapiens intercellular
adhesion molecule 5, telencephalin (ICAM5), mRNA”,
gi|12545403|ref|NM_003259.2|[12545403]; 542: NM_003269, “Homo sapiens nuclear receptor
subfamily 2, group E, member 1 (NR2E1), mRNA”, gi|21361108|ref|NM_003269.2|[21361108];
543: NM_003273, “Homo sapiens transmembrane 7 superfamily member 2 (TM7SF2),
mRNA”, gi|4507546|ref|NM_003273.1|[4507546]; 544: NM_003277, Homo sapiens claudin 5
(transmembrane protein deleted in velocardiofacial, “syndrome) (CLDN5), mRNA”,
gi|38570041|ref|NM_003277.2|[38570041]; 545: NM_003280, “Homo sapiens troponin C, slow
(TNNC1), mRNA”, gi|4507614|ref|NM_003280.1|[4507614]; 546: NM_003281, “Homo sapiens
troponin I, skeletal, slow (TNNI1), mRNA”, gi|21361554|ref|NM_003281.2|[21361554]; 547:
NM_003282, “Homo sapiens troponin I, skeletal, fast (TNNI2), mRNA”,
gi|4507620|ref|NM_003282.1|[4507620]; 548: NM_003288, “Homo sapiens tumor protein D52-
like 2 (TPD52L2), transcript variant 5, mRNA”, gi|40805859|ref|NM_003288.2|[40805859];
549: NM_003291, “Homo sapiens tripeptidyl peptidase II (TPP2), mRNA”,
gi|4507656|ref|NM_003291.1|[4507656]; 550: NM_003296, “Homo sapiens cysteine-rich
secretory protein 2 (CRISP2), mRNA”, gi|4507670|ref|NM_003296.1|[4507670]; 551:
NM_003298, “Homo sapiens nuclear receptor subfamily 2, group C, member 2 (NR2C2),
mRNA”, gi|36950990|ref|NM_003298.2|[36950990]; 552: NM_003312, “Homo sapiens
thiosulfate sulfurtransferase (rhodanese) (TST), nuclear gene”, “encoding mitochondrial protein,
mRNA”, gi|34335291|ref|NM_003312.4|[34335291]; 553: NM_003314, “Homo sapiens
tetratricopeptide repeat domain 1 (TTC1), mRNA”, gi|4507710|ref|NM_003314.1|[4507710];
554: NM_003315, “Homo sapiens DnaJ (Hsp40) homolog, subfamily C, member 7 (DNAJC7),
mRNA”, gi|4507712|ref|NM_003315.1|[4507712]; 555: NM_003323, “Homo sapiens tubby like
protein 2 (TULP2), mRNA”, gi|4507736|ref|NM_003323.1|[4507736]; 556: NM_003325, Homo
sapiens HIR histone cell cycle regulation defective homolog A (S., “cerevisiae) (HIRA),
mRNA”, gi|21536484|ref|NM_003325.3|[21536484]; 557: NM_003328, “Homo sapiens TXK
tyrosine kinase (TXK), mRNA”, gi|4507742|ref|NM_003328.1|[4507742]; 558: NM_003331,
“Homo sapiens tyrosine kinase 2 (TYK2), mRNA”, gi|34222294|ref|NM_003331.3|[34222294];
559: NM_003333, “Homo sapiens ubiquitin A-52 residue ribosomal protein fusion product 1
(UBA52),”, mRNA, gi|15451941|ref|NM_003333.2|[15451941]; 560: NM_003334, Homo
sapiens ubiquitin-activating enzyme E1 (A1S9T and BN75 temperature, “sensitivity
complementing) (UBE1), transcript variant 1, mRNA”,
gi|23510337|ref|NM_003334.2|[23510337]; 561: NM_003341, “Homo sapiens ubiquitin-
conjugating enzyme E2E 1 (UBC4/5 homolog, yeast)”, “(UBE2E1), transcript variant 1,
mRNA”, gi|33359692|ref|NM_003341.3|[33359692]; 562: NM_003350, “Homo sapiens
ubiquitin-conjugating enzyme E2 variant 2 (UBE2V2), mRNA”,
gi|12025664|ref|NM_003350.2|[12025664]; 563: NM_003369, “Homo sapiens UV radiation
resistance associated gene (UVRAG), mRNA”, gi|21687211|ref|NM_003369.2|[21687211]; 564:
NM_003374, “Homo sapiens voltage-dependent anion channel 1 (VDAC1), mRNA”,
gi|4507878|ref|NM_003374.1|[4507878]; 565: NM_003375, “Homo sapiens voltage-dependent
anion channel 2 (VDAC2), mRNA”, gi|42476280|ref|NM_003375.2|[42476280]; 566:
NM_003383, “Homo sapiens very low density lipoprotein receptor (VLDLR), mRNA”,
gi|40254472|ref|NM_003383.2|[40254472]; 567: NM_003389, “Homo sapiens coronin, actin
binding protein, 2A (CORO2A), transcript variant 1,”, mRNA,
gi|16554582|ref|NM_003389.2|[16554582]; 568: NM_003391, “Homo sapiens wingless-type
MMTV integration site family member 2 (WNT2), mRNA”,
gi|4507926|ref|NM_003391.1|[4507926]; 569: NM_003399, “Homo sapiens X-prolyl
aminopeptidase (aminopeptidase P) 2, membrane-bound”, “(XPNPEP2), mRNA”,
gi|10880125|ref|NM_003399.3|[10880125]; 570: NM_003400, “Homo sapiens exportin 1
(CRM1 homolog, yeast) (XPO1), mRNA”, gi|8051634|ref|NM_003400.2|[8051634]; 571:
NM_003404, Homo sapiens tyrosine 3-monooxygenase/tryptophan 5-monooxygenase
activation, “protein, beta polypeptide (YWHAB), transcript variant 1, mRNA”,
gi|31742479|ref|NM_003404.3|[31742479]; 572: NM_003407, “Homo sapiens zinc finger
protein 36, C3H type, homolog (mouse) (ZFP36), mRNA”,
gi|4507960|ref|NM_003407.1|[4507960]; 573: NM_003408, “Homo sapiens zinc finger protein
37 homolog (mouse) (ZFP37), mRNA”, gi|4507962|ref|NM_003408.1|[4507962]; 574:
NM_003412, “Homo sapiens Zic family member 1 (odd-paired homolog, Drosophila) (ZIC1),
mRNA”, gi|22547181|ref|NM_003412.2|[22547181]; 575: NM_003413, “Homo sapiens Zic
family member 3 heterotaxy 1 (odd-paired homolog, Drosophila)”, “(ZIC3), mRNA”,
gi|22547199|ref|NM_003413.2|[22547199]; 576: NM_003418, Homo sapiens zinc finger protein
9 (a cellular retroviral nucleic acid binding, “protein) (ZNF9), mRNA”,
gi|4827070|ref|NM_003418.1|[4827070]; 577: NM_003441, “Homo sapiens zinc finger protein
141 (clone pHZ-44) (ZNF141), mRNA”, gi|4507992|ref|NM_003441.1|[4507992]; 578:
NM_003446, “Homo sapiens zinc finger protein 157 (HZF22) (ZNF157), mRNA”,
gi|23510453|ref|NM_003446.2|[23510453]; 579: NM_003449, “Homo sapiens tripartite motif-
containing 26 (TRIM26), mRNA”, gi|16445440|ref|NM_003449.2|[16445440]; 580:
NM_003460, “Homo sapiens zona pellucida glycoprotein 2 (sperm receptor) (ZP2), mRNA”,
gi|4508044|ref|NM_003460.1|[4508044]; 581: NM_003462, “Homo sapiens dynein, axonemal,
light intermediate polypeptide 1 (DNALI1), mRNA”,
gi|37595559|ref|NM_003462.3|[37595559]; 582: NM_003468, “Homo sapiens frizzled homolog
5 (Drosophila) (FZD5), mRNA”, gi|27894384|ref|NM_003468.2|[27894384]; 583: NM_003472,
“Homo sapiens DEK oncogene (DNA binding) (DEK), mRNA”,
gi|31542502|ref|NM_003472.2|[31542502]; 584: NM_003473, Homo sapiens signal transducing
adaptor molecule (SH3 domain and ITAM motif) 1, “(STAM), mRNA”,
gi|21265027|ref|NM_003473.2|[21265027]; 585: NM_003483, “Homo sapiens high mobility
group AT-hook 2 (HMGA2), mRNA”, gi|14141182|ref|NM_003483.3|[14141182]; 586:
NM_003491, “Homo sapiens ARD1 homolog, N-acetyltransferase (S. cerevisiae) (ARD1),
mRNA”, gi|34222259|ref|NM_003491.2|[34222259]; 587: NM_003492, “Homo sapiens
chromosome X open reading frame 12 (CXorf12), mRNA”,
gi|4504738|ref|NM_003492.1|[4504738]; 588: NM_003495, “Homo sapiens histone 1, H4i
(HIST1H4I), mRNA”, gi|18105065|ref|NM_003495.2|[18105065]; 589: NM_003502, “Homo
sapiens axin 1 (AXIN1), transcript variant 1, mRNA”,
gi|31083149|ref|NM_003502.2|[31083149]; 590: NM_003504, “Homo sapiens CDC45 cell
division cycle 45-like (S. cerevisiae) (CDC45L), mRNA”,
gi|34335230|ref|NM_003504.3|[34335230]; 591: NM_003509, “Homo sapiens histone 1, H2ai
(HIST1H2AI), mRNA”, gi|15718713|ref|NM_003509.2|[15718713]; 592: NM_003524, “Homo
sapiens histone 1, H2bh (HIST1H2BH), mRNA”, gi|21166386|ref|NM_003524.2|[21166386];
593: NM_003529, “Homo sapiens histone 1, H3a (HIST1H3A), mRNA”,
gi|19743828|ref|NM_003529.2|[19743828]; 594: NM_003532, “Homo sapiens histone 1, H3e
(HIST1H3E), mRNA”, gi|21264566|ref|NM_003532.2|[21264566]; 595: NM_003536, “Homo
sapiens histone 1, H3h (HIST1H3H), mRNA”, gi|15718725|ref|NM_003536.2|[15718725]; 596:
NM_003538, “Homo sapiens histone 1, H4a (HIST1H4A), mRNA”,
gi|21166390|ref|NM_003538.3|[21166390]; 597: NM_003549, “Homo sapiens
hyaluronoglucosaminidase 3 (HYAL3), mRNA”, gi|15208650|ref|NM_003549.2|[15208650];
598: NM_003550, “Homo sapiens MAD1 mitotic arrest deficient-like 1 (yeast) (MAD1L1),
mRNA”, gi|4505064|ref|NM_003550.1|[4505064]; 599: NM_003553, “Homo sapiens olfactory
receptor, family 1, subfamily E, member 1 (OR1E1), mRNA”,
gi|11496274|ref|NM_003553.1|[11496274]; 600: NM_003554, “Homo sapiens olfactory
receptor, family 1, subfamily E, member 2 (OR1E2), mRNA”,
gi|11386152|ref|NM_003554.1|[11386152]; 601: NM_003581, “Homo sapiens NCK adaptor
protein 2 (NCK2), mRNA”, gi|4505346|ref|NM_003581.1|[4505346]; 602: NM_003582, Homo
sapiens dual-specificity tyrosine-(Y)-phosphorylation regulated kinase 3, “(DYRK3), mRNA”,
gi|4503428|ref|NM_003582.1|[4503428]; 603: NM_003585, “Homo sapiens double C2-like
domains, beta (DOC2B), mRNA”, gi|6005996|ref|NM_003585.1|[6005996]; 604: NM_003587,
“Homo sapiens DEAH (Asp-Glu-Ala-His) box polypeptide 16 (DHX16), mRNA”,
gi|21237727|ref|NM_003587.3|[21237727]; 605: NM_003592, “Homo sapiens cullin 1 (CUL1),
mRNA”, gi|32307160|ref|NM_003592.2|[32307160]; 606: NM_003594, “Homo sapiens
transcription termination factor, RNA polymerase II (TTF2), mRNA”,
gi|40807470|ref|NM_003594.3|[40807470]; 607: NM_003608, “Homo sapiens G protein-
coupled receptor 65 (GPR65), mRNA”, gi|33695103|ref|NM_003608.2|[33695103]; 608:
NM_003611, “Homo sapiens oral-facial-digital syndrome 1 (OFD1), mRNA”,
gi|4503178|ref|NM_003611.1|[4503178]; 609: NM_003614, “Homo sapiens galanin receptor 3
(GALR3), mRNA”, gi|4503906|ref|NM_003614.1|[4503906]; 610: NM_003618, “Homo sapiens
mitogen-activated protein kinase kinase kinase kinase 3 (MAP4K3),”, mRNA,
gi|15451901|ref|NM_003618.2|[15451901]; 611: NM_003625, “Homo sapiens protein tyrosine
phosphatase, receptor type, f polypeptide (PTPRF),”, “interacting protein (liprin), alpha 2
(PPFIA2), mRNA”, gi|29171754|ref|NM_003625.2|[29171754]; 612: NM_003627, “Homo
sapiens solute carrier family 43, member 1 (SLC43A1), mRNA”,
gi|42476323|ref|NM_003627.4|[42476323]; 613: NM_003632, “Homo sapiens contactin
associated protein 1 (CNTNAP1), mRNA”, gi|4505462|ref|NM_003632.1|[4505462]; 614:
NM_003635, “Homo sapiens N-deacetylase/N-sulfotransferase (heparan glucosaminyl) 2
(NDST2),”, mRNA, gi|31377809|ref|NM_003635.2|[31377809]; 615: NM_003642, “Homo
sapiens histone acetyltransferase 1 (HAT1), mRNA”, gi|4504340|ref|NM_003642.1|[4504340];
616: NM_003646, “Homo sapiens diacylglycerol kinase, zeta 104 kDa (DGKZ), transcript
variant 2,”, mRNA, gi|41872506|ref|NM_003646.2|[41872506]; 617: NM_003648, “Homo
sapiens diacylglycerol kinase, delta 130 kDa (DGKD), transcript variant 1,”, mRNA,
gi|25777595|ref|NM_003648.2|[25777595]; 618: NM_003653, Homo sapiens COP9 constitutive
photomorphogenic homolog subunit 3 (Arabidopsis), “(COPS3), mRNA”,
gi|23238221|ref|NM_003653.2|[23238221]; 619: NM_003654, “Homo sapiens carbohydrate
(keratan sulfate Gal-6) sulfotransferase 1 (CHST1),”, mRNA,
gi|31542307|ref|NM_003654.2|[31542307]; 620: NM_003655, “Homo sapiens chromobox
homolog 4 (Pc class homolog, Drosophila) (CBX4), mRNA”,
gi|4502602|ref|NM_003655.1|[4502602]; 621: NM_003656, “Homo sapiens
calcium/calmodulin-dependent protein kinase I (CAMK1), mRNA”,
gi|21536281|ref|NM_003656.3|[21536281]; 622: NM_003658, “Homo sapiens BarH-like
homeobox 2 (BARX2), mRNA”, gi|21536440|ref|NM_003658.3|[21536440]; 623: NM_003669,
“Homo sapiens inactivation escape 1 (INE1), mRNA”, gi|4504692|ref|NM_003669.1|[4504692];
624: NM_003680, “Homo sapiens tyrosyl-tRNA synthetase (YARS), mRNA”,
gi|38202242|ref|NM_003680.2|[38202242]; 625: NM_003684, “Homo sapiens MAP kinase-
interacting serine/threonine kinase 1 (MKNK1), mRNA”,
gi|34147650|ref|NM_003684.3|[34147650]; 626: NM_003686, “Homo sapiens exonuclease 1
(EXO1), transcript variant 3, mRNA”, gi|39995068|ref|NM_003686.3|[39995068]; 627:
NM_003691, “Homo sapiens serine/threonine kinase 16 (STK16), mRNA”,
gi|4505836|ref|NM_003691.1|[4505836]; 628: NM_003693, “Homo sapiens scavenger receptor
class F, member 1 (SCARF1), transcript variant”, “1, mRNA”,
gi|33598928|ref|NM_003693.2|[33598928]; 629: NM_003710, “Homo sapiens serine protease
inhibitor, Kunitz type 1 (SPINT1), transcript”, “variant 2, mRNA”,
gi|32313604|ref|NM_003710.2|[32313604]; 630: NM_003721, “Homo sapiens regulatory factor
X-associated ankyrin-containing protein (RFXANK),”, “transcript variant 1, mRNA”,
gi|19924154|ref|NM_003721.2|[19924154]; 631: NM_003729, “Homo sapiens RNA terminal
phosphate cyclase domain 1 (RTCD1), mRNA”, gi|4506588|ref|NM_003729.1|[4506588]; 632:
NM_003731, “Homo sapiens Sjogren's syndrome nuclear autoantigen 1 (SSNA1), mRNA”,
gi|4505324|ref|NM_003731.1|[4505324]; 633: NM_003733, “Homo sapiens 2′-5′-oligoadenylate
synthetase-like (OASL), transcript variant 1,”, mRNA,
gi|38016933|ref|NM_003733.2|[38016933]; 634: NM_003753, “Homo sapiens eukaryotic
translation initiation factor 3, subunit 7 zeta,”, “66/67 kDa (EIF3S7), mRNA”,
gi|23238220|ref|NM_003753.2|[23238220]; 635: NM_003755, “Homo sapiens eukaryotic
translation initiation factor 3, subunit 4 delta, 44 kDa”, “(EIF3S4), mRNA”,
gi|4503516|ref|NM_003755.1|[4503516]; 636: NM_003756, “Homo sapiens eukaryotic
translation initiation factor 3, subunit 3 gamma, 40 kDa”, “(EIF3S3), mRNA”,
gi|4503514|ref|NM_003756.1|[4503514]; 637: NM_003757, “Homo sapiens eukaryotic
translation initiation factor 3, subunit 2 beta, 36 kDa”, “(EIF3S2), mRNA”,
gi|4503512|ref|NM_003757.1|[4503512]; 638: NM_003764, “Homo sapiens syntaxin 11
(STX11), mRNA”, gi|33667037|ref|NM_003764.2|[33667037]; 639: NM_003765, “Homo
sapiens syntaxin 10 (STX10), mRNA”, gi|4507284|ref|NM_003765.1|[4507284]; 640:
NM_003771, “Homo sapiens keratin, hair, acidic, 6 (KRTHA6), mRNA”,
gi|6678648|ref|NM_003771.3|[6678648]; 641: NM_003773, “Homo sapiens
hyaluronoglucosaminidase 2 (HYAL2), transcript variant 1, mRNA”,
gi|15022800|ref|NM_003773.2|[15022800]; 642: NM_003776, “Homo sapiens mitochondrial
ribosomal protein L40 (MRPL40), nuclear gene encoding”, “mitochondrial protein, mRNA”,
gi|26638658|ref|NM_003776.2|[26638658]; 643: NM_003802, “Homo sapiens myosin, heavy
polypeptide 13, skeletal muscle (MYH13), mRNA”, gi|11321578|ref|NM_003802.1|[11321578];
644: NM_003807, “Homo sapiens tumor necrosis factor (ligand) superfamily, member 14
(TNFSF14),”, “transcript variant 1, mRNA”, gi|25952143|ref|NM_003807.2|[25952143]; 645:
NM_003815, Homo sapiens a disintegrin and metalloproteinase domain 15 (metargidin),
“(ADAM15), mRNA”, gi|11497001|ref|NM_003815.2|[11497001]; 646: NM_003816, Homo
sapiens a disintegrin and metalloproteinase domain 9 (meltrin gamma), “(ADAM9), mRNA”,
gi|4501914|ref|NM_003816.1|[4501914]; 647: NM_003819, “Homo sapiens poly(A) binding
protein, cytoplasmic 4 (inducible form) (PABPC4),”, mRNA,
gi|6552335|ref|NM_003819.2|[6552335]; 648: NM_003836, “Homo sapiens delta-like 1
homolog (Drosophila) (DLK1), mRNA”, gi|34147651|ref|NM_003836.3|[34147651]; 649:
NM_003843, “Homo sapiens sciellin (SCEL), transcript variant 1, mRNA”,
gi|21536305|ref|NM_003843.2|[21536305]; 650: NM_003849, “Homo sapiens succinate-CoA
ligase, GDP-forming, alpha subunit (SUCLG1), mRNA”,
gi|11321580|ref|NM_003849.1|[11321580]; 651: NM_003859, “Homo sapiens dolichyl-
phosphate mannosyltransferase polypeptide 1, catalytic”, “subunit (DPM1), mRNA”,
gi|4503362|ref|NM_003859.1|[4503362]; 652: NM_003860, “Homo sapiens barrier to
autointegration factor 1 (BANF1), mRNA”, gi|11038645|ref|NM_003860.2|[11038645]; 653:
NM_003863, “Homo sapiens dolichyl-phosphate mannosyltransferase polypeptide 2,
regulatory”, “subunit (DPM2), transcript variant 1, mRNA”,
gi|24497593|ref|NM_003863.2|[24497593]; 654: NM_003875, “Homo sapiens guanine
monphosphate synthetase (GMPS), mRNA”, gi|4504034|ref|NM_003875.1|[4504034]; 655:
NM_003890, “Homo sapiens Fc fragment of IgG binding protein (FCGBP), mRNA”,
gi|4503680|ref|NM_003890.1|[4503680]; 656: NM_003904, “Homo sapiens zinc finger protein
259 (ZNF259), mRNA”, gi|4508020|ref|NM_003904.1|[4508020]; 657: NM_003914, “Homo
sapiens cyclin A1 (CCNA1), mRNA”, gi|16306528|ref|NM_003914.2|[16306528]; 658:
NM_003917, “Homo sapiens adaptor-related protein complex 1, gamma 2 subunit (AP1G2),”,
“transcript variant 1, mRNA”, gi|18104994|ref|NM_003917.2|[18104994]; 659: NM_003923,
“Homo sapiens forkhead box H1 (FOXH1), mRNA”, gi|4503656|ref|NM_003923.1|[4503656];
660: NM_003924, “Homo sapiens paired-like homeobox 2b (PHOX2B), mRNA”,
gi|12707579|ref|NM_003924.2|[12707579]; 661: NM_003931, “Homo sapiens WAS protein
family, member 1 (WASF1), mRNA”, gi|4507912|ref|NM_003931.1|[4507912]; 662:
NM_003936, “Homo sapiens cyclin-dependent kinase 5, regulatory subunit 2 (p39)
(CDK5R2),”, mRNA, gi|42741664|ref|NM_003936.3|[42741664]; 663: NM_003943, “Homo
sapiens genethonin 1 (GENX-3414), mRNA”, gi|4503976|ref|NM_003943.1|[4503976]; 664:
NM_003952, “Homo sapiens ribosomal protein S6 kinase, 70 kDa, polypeptide 2 (RPS6KB2),
mRNA”, gi|4506738|ref|NM_003952.1|[4506738]; 665: NM_003957, “Homo sapiens
serine/threonine kinase 29 (STK29), mRNA”, gi|27501463|ref|NM_003957.1|[27501463]; 666:
NM_003969, “Homo sapiens ubiquitin-conjugating enzyme E2M (UBC12 homolog, yeast)
(UBE2M),”, mRNA, gi|37577133|ref|NM_003969.2|[37577133]; 667: NM_003972, “Homo
sapiens BTAF1 RNA polymerase II, B-TFIID transcription factor-associated,”, “170 kDa (Mot1
homolog, S. cerevisiae) (BTAF1), mRNA”, gi|27477069|ref|NM_003972.1|[27477069]; 668:
NM_003975, “Homo sapiens SH2 domain protein 2A (SH2D2A), mRNA”,
gi|31543620|ref|NM_003975.2|[31543620]; 669: NM_003977, “Homo sapiens aryl hydrocarbon
receptor interacting protein (AIP), mRNA”, gi|4502008|ref|NM_003977.1|[4502008]; 670:
NM_003999, “Homo sapiens oncostatin M receptor (OSMR), mRNA”,
gi|4557039|ref|NM_003999.1|[4557039]; 671: NM_004037, “Homo sapiens adenosine
monophosphate deaminase 2 (isoform L) (AMPD2), mRNA”,
gi|22507370|ref|NM_004037.5|[22507370]; 672: NM_004047, “Homo sapiens ATPase, H+
transporting, lysosomal 21 kDa, V0 subunit c″ (ATP6V0B),”, mRNA,
gi|19913434|ref|NM_004047.2|[19913434]; 673: NM_004054, “Homo sapiens complement
component 3a receptor 1 (C3AR1), mRNA”, gi|21314629|ref|NM_004054.2|[21314629]; 674:
NM_004055, “Homo sapiens calpain 5 (CAPN5), mRNA”,
gi|37577156|ref|NM_004055.3|[37577156]; 675: NM_004064, “Homo sapiens cyclin-dependent
kinase inhibitor 1B (p27, Kip1) (CDKN1B), mRNA”,
gi|17978497|ref|NM_004064.2|[17978497]; 676: NM_004073, “Homo sapiens polo-like kinase
3 (Drosophila) (PLK3), mRNA”, gi|41872373|ref|NM_004073.2|[41872373]; 677: NM_004074,
“Homo sapiens cytochrome c oxidase subunit VIII (COX8), mRNA”,
gi|4758043|ref|NM_004074.1|[4758043]; 678: NM_004078, “Homo sapiens cysteine and
glycine-rich protein 1 (CSRP1), mRNA”, gi|4758085|ref|NM_004078.1|[4758085]; 679:
NM_004083, “Homo sapiens DNA-damage-inducible transcript 3 (DDIT3), mRNA”,
gi|34147657|ref|NM_004083.3|[34147657]; 680: NM_004100, “Homo sapiens eyes absent
homolog 4 (Drosophila) (EYA4), transcript variant 1,”, mRNA,
gi|26667248|ref|NM_004100.2|[26667248]; 681: NM_004106, “Homo sapiens Fc fragment of
IgE, high affinity I, receptor for; gamma”, “polypeptide (FCER1G), mRNA”,
gi|4758343|ref|NM_004106.1|[4758343]; 682: NM_004107, “Homo sapiens Fc fragment of IgG,
receptor, transporter, alpha (FCGRT), mRNA”, gi|34222296|ref|NM_004107.3|[34222296]; 683:
NM_004110, “Homo sapiens ferredoxin reductase (FDXR), nuclear gene encoding
mitochondrial”, “protein, transcript variant 2, mRNA”,
gi|13435351|ref|NM_004110.2|[13435351]; 684: NM_004114, “Homo sapiens fibroblast growth
factor 13 (FGF13), transcript variant 1A, mRNA”, gi|16306544|ref|NM_004114.2|[16306544];
685: NM_004115, “Homo sapiens fibroblast growth factor 14 (FGF14), transcript variant 1,
mRNA”, gi|28872754|ref|NM_004115.2|[28872754]; 686: NM_004117, “Homo sapiens FK506
binding protein 5 (FKBP5), mRNA”, gi|17149847|ref|NM_004117.2|[17149847]; 687:
NM_004120, “Homo sapiens guanylate binding protein 2, interferon-inducible (GBP2),
mRNA”, gi|38327557|ref|NM_004120.3|[38327557]; 688: NM_004125, “Homo sapiens guanine
nucleotide binding protein (G protein), gamma 10 (GNG10),”, mRNA,
gi|21361096|ref|NM_004125.2|[21361096]; 689: NM_004127, “Homo sapiens G protein
pathway suppressor 1 (GPS1), mRNA”, gi|13435380|ref|NM_004127.3|[13435380]; 690:
NM_004153, “Homo sapiens origin recognition complex, subunit 1-like (yeast) (ORC1L),
mRNA”, gi|31795543|ref|NM_004153.2|[31795543]; 691: NM_004154, “Homo sapiens
pyrimidinergic receptor P2Y, G-protein coupled, 6 (P2RY6),”, “transcript variant 4, mRNA”,
gi|29029606|ref|NM_004154.3|[29029606]; 692: NM_004159, “Homo sapiens proteasome
(prosome, macropain) subunit, beta type, 8 (large”, “multifunctional protease 7) (PSMB8),
transcript variant 1, mRNA”, gi|34335277|ref|NM_004159.3|[34335277]; 693: NM_004178,
“Homo sapiens TAR (HIV) RNA binding protein 2 (TARBP2), transcript variant 3,”, mRNA,
gi|19743837|ref|NM_004178.3|[19743837]; 694: NM_004182, “Homo sapiens ubiquitously-
expressed transcript (UXT), transcript variant 2, mRNA”,
gi|24041015|ref|NM_004182.2|[24041015]; 695: NM_004188, “Homo sapiens growth factor
independent 1B (potential regulator of CDKN1A,”, “translocated in CML) (GFI1B), mRNA”,
gi|40254479|ref|NM_004188.2|[40254479]; 696: NM_004189, “Homo sapiens SRY (sex
determining region Y)-box 14 (SOX14), mRNA”, gi|31563384|ref|NM_004189.2|[31563384];
697: NM_004196, “Homo sapiens cyclin-dependent kinase-like 1 (CDC2-related kinase)
(CDKL1), mRNA”, gi|37596296|ref|NM_004196.3|[37596296]; 698: NM_004202, “Homo
sapiens thymosin, beta 4, Y-linked (TMSB4Y), mRNA”,
gi|34328944|ref|NM_004202.2|[34328944]; 699: NM_004203, Homo sapiens membrane-
associated tyrosine- and threonine-specific, “cdc2-inhibitory kinase (PKMYT1), transcript
variant 1, mRNA”, gi|33383240|ref|NM_004203.3|[33383240]; 700: NM_004204, “Homo
sapiens phosphatidylinositol glycan, class Q (PIGQ), transcript variant 2,”, mRNA,
gi|22538449|ref|NM_004204.2|[22538449]; 701: NM_004214, Homo sapiens fibroblast growth
factor (acidic) intracellular binding protein, “(FIBP), transcript variant 2, mRNA”,
gi|38683847|ref|NM_004214.4|[38683847]; 702: NM_004217, “Homo sapiens aurora kinase B
(AURKB), mRNA”, gi|4759177|ref|NM_004217.1|[4759177]; 703: NM_004219, “Homo
sapiens pituitary tumor-transforming 1 (PTTG1), mRNA”,
gi|11038651|ref|NM_004219.2|[11038651]; 704: NM_004223, “Homo sapiens ubiquitin-
conjugating enzyme E2L 6 (UBE2L6), transcript variant 1,”, mRNA,
gi|38157980|ref|NM_004223.3|[38157980]; 705: NM_004224, “Homo sapiens G protein-
coupled receptor 50 (GPR50), mRNA”, gi|4758467|ref|NM_004224.1|[4758467]; 706:
NM_004226, “Homo sapiens serine/threonine kinase 17b (apoptosis-inducing) (STK17B),
mRNA”, gi|31543661|ref|NM_004226.2|[31543661]; 707: NM_004227, “Homo sapiens
pleckstrin homology, Sec7 and coiled-coil domains 3 (PSCD3), mRNA”,
gi|33946275|ref|NM_004227.3|[33946275]; 708: NM_004233, “Homo sapiens CD83 antigen
(activated B lymphocytes, immunoglobulin superfamily)”, “(CD83), mRNA”,
gi|24475618|ref|NM_004233.2|[24475618]; 709: NM_004237, “Homo sapiens thyroid hormone
receptor interactor 13 (TRIP13), mRNA”, gi|20149561|ref|NM_004237.2|[20149561]; 710:
NM_004238,, ref|NM_004238.1|[10863902], This record was temporarily removed by RefSeq
staff for additional review.,, 711: NM_004257, “Homo sapiens transforming growth factor, beta
receptor associated protein 1”, “(TGFBRAP1), mRNA”,
gi|34222146|ref|NM_004257.3|[34222146]; 712: NM_004260, “Homo sapiens RecQ protein-
like 4 (RECQL4), mRNA”, gi|4759029|ref|NM_004260.1|[4759029]; 713: NM_004261, “Homo
sapiens 15 kDa selenoprotein (SEP15), transcript variant 1, mRNA”,
gi|42741647|ref|NM_004261.3|[42741647]; 714: NM_004267, “Homo sapiens carbohydrate (N-
acetylglucosamine-6-O) sulfotransferase 2 (CHST2),”, mRNA,
gi|27369496|ref|NM_004267.2|[27369496]; 715: NM_004272, “Homo sapiens homer homolog
1 (Drosophila) (HOMER1), mRNA”, gi|20127465|ref|NM_004272.2|[20127465]; 716:
NM_004281, “Homo sapiens BCL2-associated athanogene 3 (BAG3), mRNA”,
gi|14043023|ref|NM_004281.2|[14043023]; 717: NM_004285, “Homo sapiens hexose-6-
phosphate dehydrogenase (glucose 1-dehydrogenase) (H6PD),”, mRNA,
gi|4758497|ref|NM_004285.1|[4758497]; 718: NM_004294, “Homo sapiens mitochondrial
translational release factor 1 (MTRF1), nuclear gene”, “encoding mitochondrial protein, mRNA”,
gi|34577119|ref|NM_004294.2|[34577119]; 719: NM_004298, “Homo sapiens nucleoporin
155 kDa (NUP155), transcript variant 2, mRNA”, gi|24430147|ref|NM_004298.2|[24430147];
720: NM_004314, “Homo sapiens ADP-ribosyltransferase 1 (ART1), mRNA”,
gi|4757783|ref|NM_004314.1|[4757783]; 721: NM_004330, “Homo sapiens BCL2/adenovirus
E1B 19 kDa interacting protein 2 (BNIP2), mRNA”, gi|4757855|ref|NM_004330.1|[4757855];
722: NM_004339, “Homo sapiens pituitary tumor-transforming 1 interacting protein
(PTTG1IP), mRNA”, gi|11038670|ref|NM_004339.2|[11038670]; 723: NM_004341, “Homo
sapiens carbamoyl-phosphate synthetase 2, aspartate transcarbamylase, and”, “dihydroorotase
(CAD), mRNA”, gi|18105006|ref|NM_004341.2|[18105006]; 724: NM_004344, “Homo sapiens
centrin, EF-hand protein, 2 (CETN2), mRNA”, gi|4757901|ref|NM_004344.1|[4757901]; 725:
NM_004346, “Homo sapiens caspase 3, apoptosis-related cysteine protease (CASP3),
transcript”, “variant alpha, mRNA”, gi|14790118|ref|NM_004346.2|[14790118]; 726:
NM_004356, “Homo sapiens CD81 antigen (target of antiproliferative antibody 1) (CD81),
mRNA”, gi|21237760|ref|NM_004356.2|[21237760]; 727: NM_004357, “Homo sapiens CD151
antigen (CD151), transcript variant 1, mRNA”, gi|34328913|ref|NM_004357.3|[34328913]; 728:
NM_004358, “Homo sapiens cell division cycle 25B (CDC25B), transcript variant 1, mRNA”,
gi|11641416|ref|NM_004358.2|[11641416]; 729: NM_004359, “Homo sapiens cell division
cycle 34 (CDC34), mRNA”, gi|16357476|ref|NM_004359.1|[16357476]; 730: NM_004365,
“Homo sapiens centrin, EF-hand protein, 3 (CDC31 homolog, yeast) (CETN3), mRNA”,
gi|4757975|ref|NM_004365.1|[4757975]; 731: NM_004366, “Homo sapiens chloride channel 2
(CLCN2), mRNA”, gi|5803001|ref|NM_004366.2|[5803001]; 732: NM_004367, “Homo sapiens
chemokine (C—C motif) receptor 6 (CCR6), transcript variant 1, mRNA”,
gi|37187859|ref|NM_004367.3|[37187859]; 733: NM_004374, “Homo sapiens cytochrome c
oxidase subunit VIc (COX6C), mRNA”, gi|17999531|ref|NM_004374.2|[17999531]; 734:
NM_004383, “Homo sapiens c-src tyrosine kinase (CSK), mRNA”,
gi|4758077|ref|NM_004383.1|[4758077]; 735: NM_004396, “Homo sapiens DEAD (Asp-Glu-
Ala-Asp) box polypeptide 5 (DDX5), mRNA”, gi|13514826|ref|NM_004396.2|[13514826]; 736:
NM_004398, “Homo sapiens DEAD (Asp-Glu-Ala-Asp) box polypeptide 10 (DDX10),
mRNA”, gi|13514830|ref|NM_004398.2|[13514830]; 737: NM_004401, “Homo sapiens DNA
fragmentation factor, 45 kDa, alpha polypeptide (DFFA), mRNA”,
gi|4758147|ref|NM_004401.1|[4758147]; 738: NM_004402, “Homo sapiens DNA
fragmentation factor, 40 kDa, beta polypeptide”, “(caspase-activated DNase) (DFFB), mRNA”,
gi|4758149|ref|NM_004402.1|[4758149]; 739: NM_004411, “Homo sapiens dynein,
cytoplasmic, intermediate polypeptide 1 (DNCI1), mRNA”,
gi|4758177|ref|NM_004411.1|[4758177]; 740: NM_004415, “Homo sapiens desmoplakin
(DSP), mRNA”, gi|4758199|ref|NM_004415.1|[4758199]; 741: NM_004418, “Homo sapiens
dual specificity phosphatase 2 (DUSP2), mRNA”, gi|12707563|ref|NM_004418.2|[12707563];
742: NM_004420, “Homo sapiens dual specificity phosphatase 8 (DUSP8), mRNA”,
gi|4758211|ref|NM_004420.1|[4758211]; 743: NM_004426, “Homo sapiens polyhomeotic-like
1 (Drosophila) (PHC1), mRNA”, gi|11038623|ref|NM_004426.1|[11038623]; 744: NM_004427,
“Homo sapiens polyhomeotic-like 2 (Drosophila) (PHC2), transcript variant 2, mRNA”,
gi|37595529|ref|NM_004427.2|[37595529]; 745: NM_004432, “Homo sapiens ELAV
(embryonic lethal, abnormal vision, Drosophila)-like 2 (Hu”, “antigen B) (ELAVL2), mRNA”,
gi|4758261|ref|NM_004432.1|[4758261]; 746: NM_004438, “Homo sapiens EphA4 (EPHA4),
mRNA”, gi|32967315|ref|NM_004438.2|[32967315]; 747: NM_004445, “Homo sapiens EphB6
(EPHB6), mRNA”, gi|4758291|ref|NM_004445.1|[4758291]; 748: NM_004447, “Homo sapiens
epidermal growth factor receptor pathway substrate 8 (EPS8), mRNA”,
gi|34222299|ref|NM_004447.3|[34222299]; 749: NM_004450, “Homo sapiens enhancer of
rudimentary homolog (Drosophila) (ERH), mRNA”, gi|4758301|ref|NM_004450.1|[4758301];
750: NM_004456, “Homo sapiens enhancer of zeste homolog 2 (Drosophila) (EZH2), transcript
variant”, “1, mRNA”, gi|23510382|ref|NM_004456.3|[23510382]; 751: NM_004466, “Homo
sapiens glypican 5 (GPC5), mRNA”, gi|34106705|ref|NM_004466.3|[34106705]; 752:
NM_004469, Homo sapiens c-fos induced growth factor (vascular endothelial growth factor D),
“(FIGF), mRNA”, gi|19924297|ref|NM_004469.2|[19924297]; 753: NM_004470, “Homo
sapiens FK506 binding protein 2, 13 kDa (FKBP2), transcript variant 1, mRNA”,
gi|17149841|ref|NM_004470.2|[17149841]; 754: NM_004473, “Homo sapiens forkhead box E1
(thyroid transcription factor 2) (FOXE1), mRNA”, gi|21618324|ref|NM_004473.3|[21618324];
755: NM_004474, “Homo sapiens forkhead box D2 (FOXD2), mRNA”,
gi|4758387|ref|NM_004474.1|[4758387]; 756: NM_004480, “Homo sapiens fucosyltransferase
8 (alpha (1,6) fucosyltransferase) (FUT8),”, “transcript variant 4, mRNA”,
gi|30410721|ref|NM_004480.3|[30410721]; 757: NM_004485, “Homo sapiens guanine
nucleotide binding protein (G protein), gamma 4 (GNG4),”, mRNA,
gi|21314630|ref|NM_004485.2|[21314630]; 758: NM_004487, “Homo sapiens golgi
autoantigen, golgin subfamily b, macrogolgin (with”, “transmembrane signal), 1 (GOLGB1),
mRNA”, gi|4758453|ref|NM_004487.1|[4758453]; 759: NM_004490, “Homo sapiens growth
factor receptor-bound protein 14 (GRB14), mRNA”, gi|4758477|ref|NM_004490.1|[4758477];
760: NM_004492, “Homo sapiens general transcription factor IIA, 2 (12 kD subunit) (GTF2A2),
mRNA”, gi|4758485|ref|NM_004492.1|[4758485]; 761: NM_004496, “Homo sapiens forkhead
box A1 (FOXA1), mRNA”, gi|24497500|ref|NM_004496.2|[24497500]; 762: NM_004498,
“Homo sapiens one cut domain, family member 1 (ONECUT1), mRNA”,
gi|24307886|ref|NM_004498.1|[24307886]; 763: NM_004499, “Homo sapiens heterogeneous
nuclear ribonucleoprotein A/B (HNRPAB), transcript”, “variant 2, mRNA”,
gi|14110401|ref|NM_004499.2|[14110401]; 764: NM_004503, “Homo sapiens homeo box C6
(HOXC6), transcript variant 1, mRNA”, gi|24497542|ref|NM_004503.2|[24497542]; 765:
NM_004512, “Homo sapiens interleukin 11 receptor, alpha (IL11RA), transcript variant 1,
mRNA”, gi|22212920|ref|NM_004512.3|[22212920]; 766: NM_004524, “Homo sapiens lethal
giant larvae homolog 2 (Drosophila) (LLGL2), mRNA”,
gi|4758679|ref|NM_004524.1|[4758679]; 767: NM_004525, “Homo sapiens low density
lipoprotein-related protein 2 (LRP2), mRNA”, gi|6806918|ref|NM_004525.1|[6806918]; 768:
NM_004527, “Homo sapiens mesenchyme homeo box 1 (MEOX1), transcript variant 1,
mRNA”, gi|21396477|ref|NM_004527.2|[21396477]; 769: NM_004528, “Homo sapiens
microsomal glutathione S-transferase 3 (MGST3), mRNA”,
gi|22035640|ref|NM_004528.2|[22035640]; 770: NM_004540, “Homo sapiens neural cell
adhesion molecule 2 (NCAM2), mRNA”, gi|33519480|ref|NM_004540.2|[33519480]; 771:
NM_004542, “Homo sapiens NADH dehydrogenase (ubiquinone) 1 alpha subcomplex, 3,
9 kDa”, “(NDUFA3), mRNA”, gi|4758771|ref|NM_004542.1|[4758771]; 772: NM_004543,
“Homo sapiens nebulin (NEB), mRNA”, gi|8400716|ref|NM_004543.2|[8400716]; 773:
NM_004550, “Homo sapiens NADH dehydrogenase (ubiquinone) Fe—S protein 2, 49 kDa”,
“(NADH-coenzyme Q reductase) (NDUFS2), mRNA”,
gi|34147556|ref|NM_004550.3|[34147556]; 774: NM_004551, “Homo sapiens NADH
dehydrogenase (ubiquinone) Fe—S protein 3, 30 kDa”, “(NADH-coenzyme Q reductase)
(NDUFS3), mRNA”, gi|4758787|ref|NM_004551.1|[4758787]; 775: NM_004552, “Homo
sapiens NADH dehydrogenase (ubiquinone) Fe—S protein 5, 15 kDa”, “(NADH-coenzyme Q
reductase) (NDUFS5), mRNA”, gi|4758789|ref|NM_004552.1|[4758789]; 776: NM_004561,
“Homo sapiens ovo-like 1(Drosophila) (OVOL1), mRNA”,
gi|38570157|ref|NM_004561.2|[38570157]; 777: NM_004567, “Homo sapiens 6-phosphofructo-
2-kinase/fructose-2,6-biphosphatase 4 (PFKFB4),”, mRNA,
gi|19923257|ref|NM_004567.2|[19923257]; 778: NM_004568, “Homo sapiens serine (or
cysteine) proteinase inhibitor, clade B (ovalbumin),”, “member 6 (SERPINB6), mRNA”,
gi|41152085|ref|NM_004568.4|[41152085]; 779: NM_004569, “Homo sapiens
phosphatidylinositol glycan, class H (PIGH), mRNA”,
gi|24430187|ref|NM_004569.2|[24430187]; 780: NM_004575, “Homo sapiens POU domain,
class 4, transcription factor 2 (POU4F2), mRNA”, gi|4758947|ref|NM_004575.1|[4758947]; 781:
NM_004579, “Homo sapiens mitogen-activated protein kinase kinase kinase kinase 2
(MAP4K2),”, mRNA, gi|22035599|ref|NM_004579.2|[22035599]; 782: NM_004581, “Homo
sapiens Rab geranylgeranyltransferase, alpha subunit (RABGGTA), transcript”, “variant 2,
mRNA”, gi|33469948|ref|NM_004581.2|[33469948]; 783: NM_004584, “Homo sapiens RAD9
homolog A (S. pombe) (RAD9A), mRNA”, gi|19924112|ref|NM_004584.2|[19924112]; 784:
NM_004586, “Homo sapiens ribosomal protein S6 kinase, 90 kDa, polypeptide 3 (RPS6KA3),
mRNA”, gi|4759049|ref|NM_004586.1|[4759049]; 785: NM_004597, “Homo sapiens small
nuclear ribonucleoprotein D2 polypeptide 16.5 kDa (SNRPD2),”, “transcript variant 1, mRNA”,
gi|29294622|ref|NM_004597.4|[29294622]; 786: NM_004604, “Homo sapiens syntaxin 4A
(placental) (STX4A), mRNA”, gi|34147603|ref|NM_004604.3|[34147603]; 787: NM_004609,
“Homo sapiens transcription factor 15 (basic helix-loop-helix) (TCF15), mRNA”,
gi|38505157|ref|NM_004609.2|[38505157]; 788: NM_004612, “Homo sapiens transforming
growth factor, beta receptor I (activin A receptor”, “type II-like kinase, 53 kDa) (TGFBR1),
mRNA”, gi|4759225|ref|NM_004612.1|[4759225]; 789: NM_004619, “Homo sapiens TNF
receptor-associated factor 5 (TRAF5), transcript variant 1,”, mRNA,
gi|22027625|ref|NM_004619.2|[22027625]; 790: NM_004620, “Homo sapiens TNF receptor-
associated factor 6 (TRAF6), transcript variant 2,”, mRNA,
gi|22027628|ref|NM_004620.2|[22027628]; 791: NM_004626, “Homo sapiens wingless-type
MMTV integration site family, member 11 (WNT11), mRNA”,
gi|17017973|ref|NM_004626.2|[17017973]; 792: NM_004653, “Homo sapiens Jumonji, AT rich
interactive domain 1D (RBP2-like) (JARID1D), mRNA”,
gi|33356559|ref|NM_004653.2|[33356559]; 793: NM_004656, Homo sapiens BRCA1
associated protein-1 (ubiquitin carboxy-terminal hydrolase), “(BAP1), mRNA”,
gi|19718752|ref|NM_004656.2|[19718752]; 794: NM_004664, “Homo sapiens lin-7 homolog A
(C. elegans) (LIN7A), mRNA”, gi|4759305|ref|NM_004664.1|[4759305]; 795: NM_004666,
“Homo sapiens vanin 1 (VNN1), mRNA”, gi|4759311|ref|NM_004666.1|[4759311]; 796:
NM_004667, “Homo sapiens hect domain and RLD 2 (HERC2), mRNA”,
gi|5729867|ref|NM_004667.2|[5729867]; 797: NM_004669, “Homo sapiens chloride
intracellular channel 3 (CLIC3), mRNA”, gi|40288289|ref|NM_004669.2|[40288289]; 798:
NM_004672, “Homo sapiens mitogen-activated protein kinase kinase kinase 6 (MAP3K6),”,
“transcript variant 1, mRNA”, gi|24497521|ref|NM_004672.2|[24497521]; 799: NM_004691,
“Homo sapiens ATPase, H+ transporting, lysosomal 38 kDa, V0 subunit d isoform 1”,
“(ATP6V0D1), mRNA”, gi|34335257|ref|NM_004691.3|[34335257]; 800: NM_004693, “Homo
sapiens cytokeratin type II (K6HF), mRNA”, gi|4758617|ref|NM_004693.1|[4758617]; 801:
NM_004694, “Homo sapiens solute carrier family 16 (monocarboxylic acid transporters),
member”, “6 (SLC16A6), mRNA”, gi|40789260|ref|NM_004694.2|[40789260]; 802:
NM_004698, “Homo sapiens PRP3 pre-mRNA processing factor 3 homolog (yeast) (PRPF3),
mRNA”, gi|4758555|ref|NM_004698.1|[4758555]; 803: NM_004699, Homo sapiens DNA
segment on chromosome X (unique) 9928 expressed sequence, “(DXS9928E), mRNA”,
gi|4758219|ref|NM_004699.1|[4758219]; 804: NM_004700, “Homo sapiens potassium voltage-
gated channel, KQT-like subfamily, member 4”, “(KCNQ4), transcript variant 1, mRNA”,
gi|26638652|ref|NM_004700.2|[26638652]; 805: NM_004701, “Homo sapiens cyclin B2
(CCNB2), mRNA”, gi|10938017|ref|NM_004701.2|[10938017]; 806: NM_004704, “Homo
sapiens RNA, U3 small nucleolar interacting protein 2 (RNU3IP2), mRNA”,
gi|31543556|ref|NM_004704.2|[31543556]; 807: NM_004713, “Homo sapiens serologically
defined colon cancer antigen 1 (SDCCAG1), mRNA”,
gi|32130515|ref|NM_004713.2|[32130515]; 808: NM_004714, Homo sapiens dual-specificity
tyrosine-(Y)-phosphorylation regulated kinase 1B, “(DYRK1B), transcript variant a, mRNA”,
gi|4758221|ref|NM_004714.1|[4758221]; 809: NM_004716, “Homo sapiens proprotein
convertase subtilisin/kexin type 7 (PCSK7), mRNA”,
gi|20336247|ref|NM_004716.2|[20336247]; 810: NM_004717, “Homo sapiens diacylglycerol
kinase, iota (DGKI), mRNA”, gi|32483395|ref|NM_004717.2|[32483395]; 811: NM_004728,
“Homo sapiens DEAD (Asp-Glu-Ala-Asp) box polypeptide 21 (DDX21), mRNA”,
gi|13787208|ref|NM_004728.1|[13787208]; 812: NM_004732, “Homo sapiens potassium
voltage-gated channel, shaker-related subfamily, beta”, “member 3 (KCNAB3), mRNA”,
gi|27436970|ref|NM_004732.2|[27436970]; 813: NM_004742, “Homo sapiens BAI1-associated
protein 1 (BAIAP1), mRNA”, gi|9257194|ref|NM_004742.1|[9257194]; 814: NM_004761,
“Homo sapiens RAB2, member RAS oncogene family-like (RAB2L), mRNA”,
gi|21361071|ref|NM_004761.2|[21361071]; 815: NM_004766, “Homo sapiens coatomer protein
complex, subunit beta 2 (beta prime) (COPB2), mRNA”,
gi|4758031|ref|NM_004766.1|[4758031]; 816: NM_004767, “Homo sapiens endothelin type b
receptor-like protein 2 (ET(B)R-LP-2), mRNA”, gi|31377792|ref|NM_004767.2|[31377792];
817: NM_004784, “Homo sapiens N-deacetylase/N-sulfotransferase (heparan glucosaminyl) 3
(NDST3),”, mRNA, gi|4758765|ref|NM_004784.1|[4758765]; 818: NM_004785, “Homo
sapiens solute carrier family 9 (sodium/hydrogen exchanger), isoform 3”, “regulatory factor 2
(SLC9A3R2), mRNA”, gi|4759141|ref|NM_004785.1|[4759141]; 819: NM_004787, “Homo
sapiens slit homolog 2 (Drosophila) (SLIT2), mRNA”, gi|4759145|ref|NM_004787.1|[4759145];
820: NM_004788, “Homo sapiens ubiquitination factor E4A (UFD2 homolog, yeast) (UBE4A),
mRNA”, gi|38327028|ref|NM_004788.2|[38327028]; 821: NM_004793, “Homo sapiens
protease, serine, 15 (PRSS15), nuclear gene encoding mitochondrial”, “protein, mRNA”,
gi|21396488|ref|NM_004793.2|[21396488]; 822: NM_004800, “Homo sapiens transmembrane 9
superfamily member 2 (TM9SF2), mRNA”, gi|4758873|ref|NM_004800.1|[4758873]; 823:
NM_004804, “Homo sapiens WD40 protein Ciao1 (CIAO1), mRNA”,
gi|38570089|ref|NM_004804.2|[38570089]; 824: NM_004826, “Homo sapiens endothelin
converting enzyme-like 1 (ECEL1), mRNA”, gi|4758231|ref|NM_004826.1|[4758231]; 825:
NM_004830, “Homo sapiens cofactor required for Sp1 transcriptional activation, subunit 3,”,
“130 kDa (CRSP3), transcript variant 1, mRNA”, gi|28558970|ref|NM_004830.2|[28558970];
826: NM_004834, “Homo sapiens mitogen-activated protein kinase kinase kinase kinase 4
(MAP4K4),”, “transcript variant 1, mRNA”, gi|22035601|ref|NM_004834.2|[22035601]; 827:
NM_004836, Homo sapiens eukaryotic translation initiation factor 2-alpha kinase 3,
“(EIF2AK3), mRNA”, gi|21361154|ref|NM_004836.2|[21361154]; 828: NM_004854, “Homo
sapiens carbohydrate sulfotransferase 10 (CHST10), mRNA”,
gi|20127466|ref|NM_004854.2|[20127466]; 829: NM_004855, “Homo sapiens
phosphatidylinositol glycan, class B (PIGB), mRNA”,
gi|22538447|ref|NM_004855.3|[22538447]; 830: NM_004856, “Homo sapiens kinesin family
member 23 (KIF23), transcript variant 2, mRNA”, gi|20143965|ref|NM_004856.4|[20143965];
831: NM_004857, “Homo sapiens A kinase (PRKA) anchor protein 5 (AKAP5), mRNA”,
gi|21493042|ref|NM_004857.2|[21493042]; 832: NM_004865, “Homo sapiens TBP-like 1
(TBPL1), mRNA”, gi|21071068|ref|NM_004865.2|[21071068]; 833: NM_004869, “Homo
sapiens vacuolar protein sorting 4B (yeast) (VPS4B), mRNA”,
gi|17865801|ref|NM_004869.2|[17865801]; 834: NM_004870, “Homo sapiens mannose-P-
dolichol utilization defect 1 (MPDU1), mRNA”, gi|4759109|ref|NM_004870.1|[4759109]; 835:
NM_004872, “Homo sapiens chromosome 1 open reading frame 8 (C1orf8), mRNA”,
gi|27545320|ref|NM_004872.3|[27545320]; 836: NM_004874, “Homo sapiens BCL2-associated
athanogene 4 (BAG4), mRNA”, gi|14574569|ref|NM_004874.2|[14574569]; 837: NM_004882,
“Homo sapiens CBF1 interacting corepressor (CIR), transcript variant 1, mRNA”,
gi|40068058|ref|NM_004882.3|[40068058]; 838: NM_004891, “Homo sapiens mitochondrial
ribosomal protein L33 (MRPL33), nuclear gene encoding”, “mitochondrial protein, transcript
variant 1, mRNA”, gi|21735607|ref|NM_004891.2|[21735607]; 839: NM_004897, “Homo
sapiens multiple inositol polyphosphate histidine phosphatase, 1 (MINPP1),”, mRNA,
gi|19923760|ref|NM_004897.2|[19923760]; 840: NM_004898, “Homo sapiens clock homolog
(mouse) (CLOCK), mRNA”, gi|25777594|ref|NM_004898.2|[25777594]; 841: NM_004907,
“Homo sapiens immediate early response 2 (IER2), mRNA”,
gi|4758313|ref|NM_004907.1|[4758313]; 842: NM_004910, “Homo sapiens
phosphatidylinositol transfer protein, membrane-associated 1”, “(PITPNM1), mRNA”,
gi|4758925|ref|NM_004910.1|[4758925]; 843: NM_004913, “Homo sapiens chromosome 16
open reading frame 7 (C16orf7), mRNA”, gi|4757805|ref|NM_004913.1|[4757805]; 844:
NM_004918, “Homo sapiens T-cell leukemia/lymphoma 1B (TCL1B), transcript variant 1,
mRNA”, gi|40548373|ref|NM_004918.2|[40548373]; 845: NM_004922, “Homo sapiens SEC24
related gene family, member C (S. cerevisiae) (SEC24C),”, “transcript variant 1, mRNA”,
gi|38373668|ref|NM_004922.2|[38373668]; 846: NM_004927, “Homo sapiens mitochondrial
ribosomal protein L49 (MRPL49), nuclear gene encoding”, “mitochondrial protein, mRNA”,
gi|27436906|ref|NM_004927.2|[27436906]; 847: NM_004935, “Homo sapiens cyclin-dependent
kinase 5 (CDK5), mRNA”, gi|38454327|ref|NM_004935.2|[38454327]; 848: NM_004941,
“Homo sapiens DEAH (Asp-Glu-Ala-His) box polypeptide 8 (DHX8), mRNA”,
gi|4826689|ref|NM_004941.1|[4826689]; 849: NM_004944, “Homo sapiens deoxyribonuclease
I-like 3 (DNASE1L3), mRNA”, gi|4826697|ref|NM_004944.1|[4826697]; 850: NM_004959,
“Homo sapiens nuclear receptor subfamily 5, group A, member 1 (NR5A1), mRNA”,
gi|24432033|ref|NM_004959.3|[24432033]; 851: NM_004966, “Homo sapiens heterogeneous
nuclear ribonucleoprotein F (HNRPF), mRNA”, gi|14141150|ref|NM_004966.2|[14141150];
852: NM_004970, “Homo sapiens insulin-like growth factor binding protein, acid labile
subunit”, “(IGFALS), mRNA”, gi|4826771|ref|NM_004970.1|[4826771]; 853: NM_004974,
“Homo sapiens potassium voltage-gated channel, shaker-related subfamily, member 2”,
“(KCNA2), mRNA”, gi|25952079|ref|NM_004974.2|[25952079]; 854: NM_004975, “Homo
sapiens potassium voltage-gated channel, Shab-related subfamily, member 1”, “(KCNB1),
mRNA”, gi|27436972|ref|NM_004975.2|[27436972]; 855: NM_004978, “Homo sapiens
potassium voltage-gated channel, Shaw-related subfamily, member 4”, “(KCNC4), transcript
variant 1, mRNA”, gi|24497461|ref|NM_004978.2|[24497461]; 856: NM_004984, “Homo
sapiens kinesin family member 5A (KIF5A), mRNA”, gi|4826807|ref|NM_004984.1|[4826807];
857: NM_004987, “Homo sapiens LIM and senescent cell antigen-like domains 1 (LIMS1),
mRNA”, gi|13518025|ref|NM_004987.2|[13518025]; 858: NM_004991, “Homo sapiens
myelodysplasia syndrome 1 (MDS1), mRNA”, gi|4826827|ref|NM_004991.1|[4826827]; 859:
NM_004994, “Homo sapiens matrix metalloproteinase 9 (gelatinase B, 92 kDa gelatinase,
92 kDa”, “type IV collagenase) (MMP9), mRNA”, gi|4826835|ref|NM_004994.1|[4826835]; 860:
NM_004998, “Homo sapiens myosin IE (MYO1E), mRNA”,
gi|4826843|ref|NM_004998.1|[4826843]; 861: NM_005006, “Homo sapiens NADH
dehydrogenase (ubiquinone) Fe—S protein 1, 75 kDa”, “(NADH-coenzyme Q reductase)
(NDUFS1), nuclear gene encoding mitochondrial”, “protein, mRNA”,
gi|33519474|ref|NM_005006.5|[33519474]; 862: NM_005007, Homo sapiens nuclear factor of
kappa light polypeptide gene enhancer in B-cells, “inhibitor-like 1 (NFKBIL1), mRNA”,
gi|26787990|ref|NM_005007.2|[26787990]; 863: NM_005012, “Homo sapiens receptor tyrosine
kinase-like orphan receptor 1 (ROR1), mRNA”, gi|4826867|ref|NM_005012.1|[4826867]; 864:
NM_005017, “Homo sapiens phosphate cytidylyltransferase 1, choline, alpha isoform
(PCYT1A),”, mRNA, gi|31543384|ref|NM_005017.2|[31543384]; 865: NM_005023, “Homo
sapiens protein geranylgeranyltransferase type I, beta subunit (PGGT1B),”, mRNA,
gi|27597101|ref|NM_005023.2|[27597101]; 866: NM_005025, “Homo sapiens serine (or
cysteine) proteinase inhibitor, clade I (neuroserpin),”, “member 1 (SERPINI1), mRNA”,
gi|4826903|ref|NM_005025.1|[4826903]; 867: NM_005027, “Homo sapiens phosphoinositide-3-
kinase, regulatory subunit, polypeptide 2 (p85”, “beta) (PIK3R2), mRNA”,
gi|4826907|ref|NM_005027.1|[4826907]; 868: NM_005028, “Homo sapiens
phosphatidylinositol-4-phosphate 5-kinase, type II, alpha”, “(PIP5K2A), mRNA”,
gi|20302162|ref|NM_005028.3|[20302162]; 869: NM_005037, “Homo sapiens peroxisome
proliferative activated receptor, gamma (PPARG),”, “transcript variant 4, mRNA”,
gi|20336230|ref|NM_005037.3|[20336230]; 870: NM_005041, “Homo sapiens perforin 1 (pore
forming protein) (PRF1), mRNA”, gi|40254807|ref|NM_005041.2|[40254807]; 871:
NM_005048, “Homo sapiens parathyroid hormone receptor 2 (PTHR2), mRNA”,
gi|39995097|ref|NM_005048.2|[39995097]; 872: NM_005049, “Homo sapiens PWP2 periodic
tryptophan protein homolog (yeast) (PWP2H), mRNA”,
gi|4826955|ref|NM_005049.1|[4826955]; 873: NM_005051, “Homo sapiens glutaminyl-tRNA
synthetase (QARS), mRNA”, gi|4826959|ref|NM_005051.1|[4826959]; 874: NM_005074,
“Homo sapiens solute carrier family 17 (sodium phosphate), member 1 (SLC17A1),”, mRNA,
gi|4827009|ref|NM_005074.1|[4827009]; 875: NM_005076, “Homo sapiens contactin 2 (axonal)
(CNTN2), mRNA”, gi|28373120|ref|NM_005076.2|[28373120]; 876: NM_005084, “Homo
sapiens phospholipase A2, group VII (platelet-activating factor”, “acetylhydrolase, plasma)
(PLA2G7), mRNA”, gi|31543409|ref|NM_005084.2|[31543409]; 877: NM_005092, “Homo
sapiens tumor necrosis factor (ligand) superfamily, member 18 (TNFSF18),”, mRNA,
gi|40354198|ref|NM_005092.2|[40354198]; 878: NM_005097, “Homo sapiens leucine-rich,
glioma inactivated 1 (LGI1), mRNA”, gi|4826815|ref|NM_005097.1|[4826815]; 879:
NM_005098, “Homo sapiens musculin (activated B-cell factor-1) (MSC), mRNA”,
gi|6996017|ref|NM_005098.2|[6996017]; 880: NM_005113, “Homo sapiens golgi autoantigen,
golgin subfamily a, 5 (GOLGA5), mRNA”, gi|30260187|ref|NM_005113.2|[30260187]; 881:
NM_005124, “Homo sapiens nucleoporin 153 kDa (NUP153), mRNA”,
gi|24430145|ref|NM_005124.2|[24430145]; 882: NM_005125, “Homo sapiens copper
chaperone for superoxide dismutase (CCS), mRNA”, gi|4826664|ref|NM_005125.1|[4826664];
883: NM_005132, “Homo sapiens REC8-like 1 (yeast) (REC8L1), mRNA”,
gi|9845292|ref|NM_005132.1|[9845292]; 884: NM_005139, “Homo sapiens annexin A3
(ANXA3), mRNA”, gi|4826642|ref|NM_005139.1|[4826642]; 885: NM_005146, “Homo
sapiens squamous cell carcinoma antigen recognised by T cells (SART1), mRNA”,
gi|38788009|ref|NM_005146.3|[38788009]; 886: NM_005147, “Homo sapiens DnaJ (Hsp40)
homolog, subfamily A, member 3 (DNAJA3), mRNA”,
gi|40786390|ref|NM_005147.3|[40786390]; 887: NM_005154, “Homo sapiens ubiquitin specific
protease 8 (USP8), mRNA”, gi|41281375|ref|NM_005154.2|[41281375]; 888: NM_005161,
“Homo sapiens angiotensin II receptor-like 1 (AGTRL1), mRNA”,
gi|34577064|ref|NM_005161.2|[34577064]; 889: NM_005164, “Homo sapiens ATP-binding
cassette, sub-family D (ALD), member 2 (ABCD2), mRNA”,
gi|21536379|ref|NM_005164.2|[21536379]; 890: NM_005169, “Homo sapiens paired-like
(aristaless) homeobox 2a (PHOX2A), mRNA”, gi|4885070|ref|NM_005169.1|[4885070]; 891:
NM_005170, “Homo sapiens achaete-scute complex-like 2 (Drosophila) (ASCL2), mRNA”,
gi|42716308|ref|NM_005170.2|[42716308]; 892: NM_005171, “Homo sapiens activating
transcription factor 1 (ATF1), mRNA”, gi|38261963|ref|NM_005171.2|[38261963]; 893:
NM_005182, “Homo sapiens carbonic anhydrase VII (CA7), mRNA”,
gi|4885100|ref|NM_005182.1|[4885100]; 894: NM_005186, “Homo sapiens calpain 1, (mu/I)
large subunit (CAPN1), mRNA”, gi|12408655|ref|NM_005186.2|[12408655]; 895: NM_005198,
“Homo sapiens choline kinase-like (CHKL), transcript variant 1, mRNA”,
gi|23238259|ref|NM_005198.3|[23238259]; 896: NM_005209, “Homo sapiens crystallin, beta
A2 (CRYBA2), transcript variant 1, mRNA”, gi|7019356|ref|NM_005209.1|[7019356]; 897:
NM_005215, “Homo sapiens deleted in colorectal carcinoma (DCC), mRNA”,
gi|4885174|ref|NM_005215.1|[4885174]; 898: NM_005221, “Homo sapiens distal-less homeo
box 5 (DLX5), mRNA”, gi|41352719|ref|NM_005221.4|[41352719]; 899: NM_005222,,
ref|NM_005222.1|DLX6[4885188], This record was temporarily removed by RefSeq staff for
additional review.,, 900: NM_005223, “Homo sapiens deoxyribonuclease I (DNASE1),
mRNA”, gi|21361253|ref|NM_005223.2|[21361253]; 901: NM_005224, “Homo sapiens AT rich
interactive domain 3A (BRIGHT-like) (ARID3A), mRNA”,
gi|4885192|ref|NM_005224.1|[4885192]; 902: NM_005227, “Homo sapiens ephrin-A4
(EFNA4), transcript variant 1, mRNA”, gi|33359684|ref|NM_005227.2|[33359684]; 903:
NM_005236, “Homo sapiens excision repair cross-complementing rodent repair deficiency,”,
“complementation group 4 (ERCC4), mRNA”, gi|4885216|ref|NM_005236.1|[4885216]; 904:
NM_005238, “Homo sapiens v-ets erythroblastosis virus E26 oncogene homolog 1 (avian)
(ETS1),”, mRNA, gi|41393580|ref|NM_005238.2|[41393580]; 905: NM_005239, “Homo
sapiens v-ets erythroblastosis virus E26 oncogene homolog 2 (avian) (ETS2),”, mRNA,
gi|20127471|ref|NM_005239.2|[20127471]; 906: NM_005245, “Homo sapiens FAT tumor
suppressor homolog 1 (Drosophila) (FAT), mRNA”, gi|4885228|ref|NM_005245.1|[4885228];
907: NM_005246, “Homo sapiens fer (fps/fes related) tyrosine kinase (phosphoprotein NCP94)
(FER),”, mRNA, gi|4885230|ref|NM_005246.1|[4885230]; 908: NM_005251, “Homo sapiens
forkhead box C2 (MFH-1, mesenchyme forkhead 1) (FOXC2), mRNA”,
gi|4885236|ref|NM_005251.1|[4885236]; 909: NM_005256, “Homo sapiens growth arrest-
specific 2 (GAS2), transcript variant 1, mRNA”, gi|29540560|ref|NM_005256.2|[29540560];
910: NM_005257, “Homo sapiens GATA binding protein 6 (GATA6), mRNA”,
gi|40288196|ref|NM_005257.3|[40288196]; 911: NM_005258, “Homo sapiens GTP
cyclohydrolase I feedback regulatory protein (GCHFR), mRNA”,
gi|6382072|ref|NM_005258.2|[6382072]; 912: NM_005260, “Homo sapiens growth
differentiation factor 9 (GDF9), mRNA”, gi|6715598|ref|NM_005260.2|[6715598]; 913:
NM_005264, “Homo sapiens GDNF family receptor alpha 1 (GFRA1), transcript variant 1,
mRNA”, gi|22035690|ref|NM_005264.2|[22035690]; 914: NM_005266, “Homo sapiens gap
junction protein, alpha 5, 40 kDa (connexin 40) (GJA5),”, “transcript variant A, mRNA”,
gi|32483413|ref|NM_005266.4|[32483413]; 915: NM_005268, “Homo sapiens gap junction
protein, beta 5 (connexin 31.1) (GJB5), mRNA”, gi|31542847|ref|NM_005268.2|[31542847];
916: NM_005272, “Homo sapiens guanine nucleotide binding protein (G protein), alpha
transducing”, “activity polypeptide 2 (GNAT2), mRNA”,
gi|22027523|ref|NM_005272.2|[22027523]; 917: NM_005275, “Homo sapiens guanine
nucleotide binding protein-like 1 (GNL1), mRNA”, gi|38788318|ref|NM_005275.2|[38788318];
918: NM_005281, “Homo sapiens G protein-coupled receptor 3 (GPR3), mRNA”,
gi|31377791|ref|NM_005281.2|[31377791]; 919: NM_005286, “Homo sapiens G protein-
coupled receptor 8 (GPR8), mRNA”, gi|30581163|ref|NM_005286.2|[30581163]; 920:
NM_005288, “Homo sapiens G protein-coupled receptor 12 (GPR12), mRNA”,
gi|4885294|ref|NM_005288.1|[4885294]; 921: NM_005299, “Homo sapiens G protein-coupled
receptor 31 (GPR31), mRNA”, gi|4885316|ref|NM_005299.1|[4885316]; 922: NM_005302,
Homo sapiens G protein-coupled receptor 37 (endothelin receptor type B-like), “(GPR37),
mRNA”, gi|31377788|ref|NM_005302.2|[31377788]; 923: NM_005306, “Homo sapiens G
protein-coupled receptor 43 (GPR43), mRNA”, gi|4885332|ref|NM_005306.1|[4885332]; 924:
NM_005309, “Homo sapiens glutamic-pyruvate transaminase (alanine aminotransferase)
(GPT),”, mRNA, gi|4885350|ref|NM_005309.1|[4885350]; 925: NM_005312, Homo sapiens
guanine nucleotide-releasing factor 2 (specific for crk, “proto-oncogene) (GRF2), transcript
variant 1, mRNA”, gi|38373674|ref|NM_005312.2|[38373674]; 926: NM_005313, “Homo
sapiens glucose regulated protein, 58 kDa (GRP58), mRNA”,
gi|21361656|ref|NM_005313.3|[21361656]; 927: NM_005318, “Homo sapiens H1 histone
family, member 0 (H1F0), mRNA”, gi|20336758|ref|NM_005318.2|[20336758]; 928:
NM_005321, “Homo sapiens histone 1, H1e (HIST1H1E), mRNA”,
gi|20544164|ref|NM_005321.2|[20544164]; 929: NM_005325, “Homo sapiens histone 1, H1a
(HIST1H1A), mRNA”, gi|21264571|ref|NM_005325.2|[21264571]; 930: NM_005330, “Homo
sapiens hemoglobin, epsilon 1 (HBE1), mRNA”, gi|28302129|ref|NM_005330.3|[28302129];
931: NM_005341, “Homo sapiens GLI-Kruppel family member HKR3 (HKR3), mRNA”,
gi|4885418|ref|NM_005341.1|[4885418]; 932: NM_005370, “Homo sapiens RAB8A, member
RAS oncogene family (RAB8A), mRNA”, gi|40548385|ref|NM_005370.4|[40548385]; 933:
NM_005379, “Homo sapiens myosin IA (MYO1A), mRNA”,
gi|29544746|ref|NM_005379.2|[29544746]; 934: NM_005381, “Homo sapiens nucleolin (NCL),
mRNA”, gi|4885510|ref|NM_005381.1|[4885510]; 935: NM_005382, “Homo sapiens
neurofilament 3 (150 kDa medium) (NEF3), mRNA”, gi|4885512|ref|NM_005382.1|[4885512];
936: NM_005386, “Homo sapiens neuronatin (NNAT), transcript variant 1, mRNA”,
gi|32307134|ref|NM_005386.2|[32307134]; 937: NM_005390, “Homo sapiens pyruvate
dehydrogenase (lipoamide) alpha 2 (PDHA2), mRNA”,
gi|38492354|ref|NM_005390.3|[38492354]; 938: NM_005393, “Homo sapiens plexin B3
(PLXNB3), mRNA”, gi|10864080|ref|NM_005393.1|[10864080]; 939: NM_005398, “Homo
sapiens protein phosphatase 1, regulatory (inhibitor) subunit 3C (PPP1R3C),”, mRNA,
gi|42476161|ref|NM_005398.3|[42476161]; 940: NM_005401, “Homo sapiens protein tyrosine
phosphatase, non-receptor type 14 (PTPN14), mRNA”,
gi|34328898|ref|NM_005401.3|[34328898]; 941: NM_005402, Homo sapiens v-ral simian
leukemia viral oncogene homolog A (ras related), “(RALA), mRNA”,
gi|33946328|ref|NM_005402.2|[33946328]; 942: NM_005418, “Homo sapiens suppression of
tumorigenicity 5 (ST5), transcript variant 1, mRNA”,
gi|21264611|ref|NM_005418.2|[21264611]; 943: NM_005423, “Homo sapiens trefoil factor 2
(spasmolytic protein 1) (TFF2), mRNA”, gi|38488723|ref|NM_005423.2|[38488723]; 944:
NM_005424, Homo sapiens tyrosine kinase with immunoglobulin and epidermal growth factor,
“homology domains (TIE), mRNA”, gi|31543809|ref|NM_005424.2|[31543809]; 945:
NM_005426, “Homo sapiens tumor protein p53 binding protein, 2 (TP53BP2), mRNA”,
gi|4885642|ref|NM_005426.1|[4885642]; 946: NM_005427, “Homo sapiens tumor protein p73
(TP73), mRNA”, gi|4885644|ref|NM_005427.1|[4885644]; 947: NM_005428, “Homo sapiens
vav 1 oncogene (VAV1), mRNA”, gi|7108366|ref|NM_005428.2|[7108366]; 948: NM_005429,
“Homo sapiens vascular endothelial growth factor C (VEGFC), mRNA”,
gi|19924300|ref|NM_005429.2|[19924300]; 949: NM_005431, Homo sapiens X-ray repair
complementing defective repair in Chinese hamster, “cells 2 (XRCC2), mRNA”,
gi|4885656|ref|NM_005431.1|[4885656]; 950: NM_005432, Homo sapiens X-ray repair
complementing defective repair in Chinese hamster, “cells 3 (XRCC3), mRNA”,
gi|12408644|ref|NM_005432.2|[12408644]; 951: NM_005436, “Homo sapiens coiled-coil
domain containing 6 (CCDC6), mRNA”, gi|4885172|ref|NM_005436.1|[4885172]; 952:
NM_005439, “Homo sapiens myeloid leukemia factor 2 (MLF2), mRNA”,
gi|4885486|ref|NM_005439.1|[4885486]; 953: NM_005441, “Homo sapiens chromatin
assembly factor 1, subunit B (p60) (CHAF1B), mRNA”,
gi|4885104|ref|NM_005441.1|[4885104]; 954: NM_005452, “Homo sapiens chromosome 6
open reading frame 11 (C6orf11), mRNA”, gi|39725662|ref|NM_005452.4|[39725662]; 955:
NM_005453, “Homo sapiens zinc finger protein 297 (ZNF297), mRNA”,
gi|20070223|ref|NM_005453.3|[20070223]; 956: NM_005460, “Homo sapiens synuclein, alpha
interacting protein (synphilin) (SNCAIP), mRNA”, gi|4885602|ref|NM_005460.1|[4885602];
957: NM_005461, Homo sapiens v-maf musculoaponeurotic fibrosarcoma oncogene homolog B
(avian), “(MAFB), mRNA”, gi|31652256|ref|NM_005461.3|[31652256]; 958: NM_005469,
“Homo sapiens peroxisomal acyl-CoA thioesterase (PTE1), transcript variant 1,”, mRNA,
gi|34577074|ref|NM_005469.2|[34577074]; 959: NM_005474, “Homo sapiens histone
deacetylase 5 (HDAC5), transcript variant 1, mRNA”,
gi|21237796|ref|NM_005474.3|[21237796]; 960: NM_005475, “Homo sapiens lymphocyte
adaptor protein (LNK), mRNA”, gi|4885454|ref|NM_005475.1|[4885454]; 961: NM_005477,
Homo sapiens hyperpolarization activated cyclic nucleotide-gated potassium, “channel 4
(HCN4), mRNA”, gi|4885406|ref|NM_005477.1|[4885406]; 962: NM_005479, “Homo sapiens
frequently rearranged in advanced T-cell lymphomas (FRAT1),”, “transcript variant 1, mRNA”,
gi|31317235|ref|NM_005479.2|[31317235]; 963: NM_005485, Homo sapiens ADP-
ribosyltransferase (NAD+; poly (ADP-ribose) polymerase)-like 3, “(ADPRTL3), mRNA”,
gi|11496992|ref|NM_005485.2|[11496992]; 964: NM_005490, “Homo sapiens SH2 domain
containing 3A (SH2D3A), mRNA”, gi|4885524|ref|NM_005490.1|[4885524]; 965: NM_005499
“Homo sapiens SUMO-1 activating enzyme subunit 2 (UBA2), mRNA”,
gi|4885648|ref|NM_005499.1|[4885648]; 966: NM_005505, “Homo sapiens scavenger receptor
class B, member 1 (SCARB1), mRNA”, gi|33620766|ref|NM_005505.3|[33620766]; 967:
NM_005507, “Homo sapiens cofilin 1 (non-muscle) (CFL1), mRNA”,
gi|5031634|ref|NM_005507.1|[5031634]; 968: NM_005517, “Homo sapiens high-mobility
group nucleosomal binding domain 2 (HMGN2), mRNA”,
gi|5031748|ref|NM_005517.1|[5031748]; 969: NM_005522, “Homo sapiens homeo box A1
(HOXA1), transcript variant 1, mRNA”, gi|24497507|ref|NM_005522.3|[24497507]; 970:
NM_005527, “Homo sapiens heat shock 70 kDa protein 1-like (HSPA1L), mRNA”,
gi|27436928|ref|NM_005527.2|[27436928]; 971: NM_005534, Homo sapiens interferon gamma
receptor 2 (interferon gamma transducer 1), “(IFNGR2), mRNA”,
gi|5031782|ref|NM_005534.1|[5031782]; 972: NM_005536, “Homo sapiens inositol(myo)-1(or
4)-monophosphatase 1 (IMPA1), mRNA”, gi|8393607|ref|NM_005536.2|[8393607]; 973:
NM_005539, “Homo sapiens inositol polyphosphate-5-phosphatase, 40 kDa (INPP5A), mRNA”,
gi|38327536|ref|NM_005539.2|[38327536]; 974: NM_005545, “Homo sapiens immunoglobulin
superfamily containing leucine-rich repeat (ISLR),”, “transcript variant 1, mRNA”,
gi|41582237|ref|NM_005545.3|[41582237]; 975: NM_005550, “Homo sapiens kinesin family
member C3 (KIFC3), mRNA”, gi|19923320|ref|NM_005550.2|[19923320]; 976: NM_005560,
“Homo sapiens laminin, alpha 5 (LAMA5), mRNA”, gi|21264601|ref|NM_005560.3|[21264601];
977: NM_005563, “Homo sapiens stathmin 1/oncoprotein 18 (STMN1), mRNA”,
gi|13518023|ref|NM_005563.2|[13518023]; 978: NM_005567, “Homo sapiens lectin,
galactoside-binding, soluble, 3 binding protein (LGALS3BP),”, mRNA,
gi|6006016|ref|NM_005567.2|[6006016]; 979: NM_005574, “Homo sapiens LIM domain only 2
(rhombotin-like 1) (LMO2), mRNA”, gi|6633806|ref|NM_005574.2|[6633806]; 980:
NM_005575, “Homo sapiens leucyl/cystinyl aminopeptidase (LNPEP), mRNA”,
gi|5031880|ref|NM_005575.1|[5031880]; 981: NM_005583, “Homo sapiens lymphoblastic
leukemia derived sequence 1 (LYL1), mRNA”, gi|34147557|ref|NM_005583.3|[34147557]; 982:
NM_005584, “Homo sapiens mab-21-like 1 (C. elegans) (MAB21L1), mRNA”,
gi|18765719|ref|NM_005584.2|[18765719]; 983: NM_005608, “Homo sapiens protein tyrosine
phosphatase, receptor type, C-associated protein”, “(PTPRCAP), mRNA”,
gi|5032004|ref|NM_005608.1|[5032004]; 984: NM_005620, “Homo sapiens S100 calcium
binding protein A11 (calgizzarin) (S100A11), mRNA”, gi|5032056|ref|NM_005620.1|[5032056];
985: NM_005626, “Homo sapiens splicing factor, arginine/serine-rich 4 (SFRS4), mRNA”,
gi|34147660|ref|NM_005626.3|[34147660]; 986: NM_005627, “Homo sapiens
serum/glucocorticoid regulated kinase (SGK), mRNA”,
gi|25168262|ref|NM_005627.2|[25168262]; 987: NM_005628, “Homo sapiens solute carrier
family 1 (neutral amino acid transporter), member 5”, “(SLC1A5), mRNA”,
gi|5032092|ref|NM_005628.1|[5032092]; 988: NM_005632, “Homo sapiens small optic lobes
homolog (Drosophila) (SOLH), mRNA”, gi|41406087|ref|NM_005632.2|[41406087]; 989:
NM_005634, “Homo sapiens SRY (sex determining region Y)-box 3 (SOX3), mRNA”,
gi|30061555|ref|NM_005634.2|[30061555]; 990: NM_005643, “Homo sapiens TAF11 RNA
polymerase II, TATA box binding protein (TBP)-associated”, “factor, 28 kDa (TAF11), mRNA”,
gi|21269863|ref|NM_005643.2|[21269863]; 991: NM_005644, “Homo sapiens TAF12 RNA
polymerase II, TATA box binding protein (TBP)-associated”, “factor, 20 kDa (TAF12), mRNA”,
gi|9943840|ref|NM_005644.2|[9943840]; 992: NM_005652, “Homo sapiens telomeric repeat
binding factor 2 (TERF2), mRNA”, gi|21536372|ref|NM_005652.2|[21536372]; 993:
NM_005655, “Homo sapiens TGFB inducible early growth response (TIEG), mRNA”,
gi|5032176|ref|NM_005655.1|[5032176]; 994: NM_005657, “Homo sapiens tumor protein p53
binding protein, 1 (TP53BP1), mRNA”, gi|5032188|ref|NM_005657.1|[5032188]; 995:
NM_005659, “Homo sapiens ubiquitin fusion degradation 1-like (UFD1L), mRNA”,
gi|34222257|ref|NM_005659.3|[34222257]; 996: NM_005664, “Homo sapiens makorin, ring
finger protein, 3 (MKRN3), mRNA”, gi|5032242|ref|NM_005664.1|[5032242]; 997:
NM_005671, “Homo sapiens reproduction 8 (D8S2298E), mRNA”,
gi|5031650|ref|NM_005671.1|[5031650]; 998: NM_005688, “Homo sapiens ATP-binding
cassette, sub-family C (CFTR/MRP), member 5 (ABCC5),”, mRNA,
gi|5032100|ref|NM_005688.1|[5032100]; 999: NM_005690, “Homo sapiens dynamin 1-like
(DNM1L), transcript variant 3, mRNA”, gi|6996008|ref|NM_005690.2|[6996008]; 1000:
NM_005694, “Homo sapiens COX17 homolog, cytochrome c oxidase assembly protein
(yeast)”, “(COX17), nuclear gene encoding mitochondrial protein, mRNA”,
gi|5031644|ref|NM_005694.1|[5031644]; 1001: NM_005697, “Homo sapiens secretory carrier
membrane protein 2 (SCAMP2), mRNA”, gi|16445417|ref|NM_005697.3|[16445417]; 1002:
NM_005698, “Homo sapiens secretory carrier membrane protein 3 (SCAMP3), transcript
variant”, “1, mRNA”, gi|16445418|ref|NM_005698.2|[16445418]; 1003: NM_005700, “Homo
sapiens dipeptidylpeptidase 3 (DPP3), transcript variant 1, mRNA”,
gi|18491023|ref|NM_005700.2|[18491023]; 1004: NM_005705, “Homo sapiens pan-
hematopoietic expression (PHEMX), transcript variant 2, mRNA”,
gi|37595533|ref|NM_005705.3|[37595533]; 1005: NM_005706, “Homo sapiens tumor
suppressing subtransferable candidate 4 (TSSC4), mRNA”,
gi|21071005|ref|NM_005706.2|[21071005]; 1006: NM_005713, “Homo sapiens collagen, type
IV, alpha 3 (Goodpasture antigen) binding protein”, “(COL4A3BP), transcript variant 1,
mRNA”, gi|5031716|ref|NM_005713.1|[5031716]; 1007: NM_005714, “Homo sapiens
potassium channel, subfamily K, member 7 (KCNK7), transcript”, “variant C, mRNA”,
gi|5031820|ref|NM_005714.1|[5031820]; 1008: NM_005716, Homo sapiens regulator of G-
protein signalling 19 interacting protein 1, “(RGS19IP1), transcript variant 1, mRNA”,
gi|42544147|ref|NM_005716.2|[42544147]; 1009: NM_005717, “Homo sapiens actin related
protein 2/3 complex, subunit 5, 16 kDa (ARPC5), mRNA”,
gi|23238212|ref|NM_005717.2|[23238212]; 1010: NM_005719, “Homo sapiens actin related
protein 2/3 complex, subunit 3, 21 kDa (ARPC3), mRNA”,
gi|23397667|ref|NM_005719.2|[23397667]; 1011: NM_005726, “Homo sapiens Ts translation
elongation factor, mitochondrial (TSFM), mRNA”, gi|21361279|ref|NM_005726.2|[21361279];
1012: NM_005727, “Homo sapiens tetraspan 1 (TSPAN-1), mRNA”,
gi|21264577|ref|NM_005727.2|[21264577]; 1013: NM_005738, “Homo sapiens ADP-
ribosylation factor-like 4 (ARL4), mRNA”, gi|5031602|ref|NM_005738.1|[5031602]; 1014:
NM_005740, “Homo sapiens dynein, axonemal, light polypeptide 4 (DNAL4), mRNA”,
gi|5031666|ref|NM_005740.1|[5031666]; 1015: NM_005745, “Homo sapiens B-cell receptor-
associated protein 31 (BCAP31), mRNA”, gi|32171185|ref|NM_005745.5|[32171185]; 1016:
NM_005755, “Homo sapiens Epstein-Barr virus induced gene 3 (EBI3), mRNA”,
gi|14577916|ref|NM_005755.2|[14577916]; 1017: NM_005756, “Homo sapiens G protein-
coupled receptor 64 (GPR64), mRNA”, gi|5031732|ref|NM_005756.1|[5031732]; 1018:
NM_005764, “Homo sapiens membrane-associated protein 17 (MAP17), mRNA”,
gi|41152089|ref|NM_005764.3|[41152089]; 1019: NM_005770, “Homo sapiens small EDRK-
rich factor 2 (SERF2), mRNA”, gi|42475556|ref|NM_005770.3|[42475556]; 1020: NM_005772,
“Homo sapiens RNA terminal phosphate cyclase-like 1 (RCL1), mRNA”,
gi|21361284|ref|NM_005772.2|[21361284]; 1021: NM_005780, “Homo sapiens lipoma HMGIC
fusion partner (LHFP), mRNA”, gi|5031864|ref|NM_005780.1|[5031864]; 1022: NM_005787,
“Homo sapiens asparagine-linked glycosylation 3 homolog (yeast,”, “alpha-1,3-
mannosyltransferase) (ALG3), mRNA”, gi|39725713|ref|NM_005787.3|[39725713]; 1023:
NM_005792, “Homo sapiens M-phase phosphoprotein 6 (MPHOSPH6), mRNA”,
gi|5031918|ref|NM_005792.1|[5031918]; 1024: NM_005796, “Homo sapiens nuclear transport
factor 2 (NUTF2), mRNA”, gi|5031984|ref|NM_005796.1|[5031984]; 1025: NM_005798,
“Homo sapiens ret finger protein 2 (RFP2), transcript variant 1, mRNA”,
gi|16445410|ref|NM_005798.2|[16445410]; 1026: NM_005805, “Homo sapiens proteasome
(prosome, macropain) 26S subunit, non-ATPase, 14”, “(PSMD14), mRNA”,
gi|42734423|ref|NM_005805.2|[42734423]; 1027: NM_005833, “Homo sapiens Rab9 effector
p40 (RAB9P40), mRNA”, gi|33695108|ref|NM_005833.2|[33695108]; 1028: NM_005835,
“Homo sapiens solute carrier family 17 (sodium phosphate), member 2 (SLC17A2),”, mRNA,
gi|5031954|ref|NM_005835.1|[5031954]; 1029: NM_005836, “Homo sapiens translational
inhibitor protein p14.5 (UK114), mRNA”, gi|5032214|ref|NM_005836.1|[5032214]; 1030:
NM_005842, “Homo sapiens sprouty homolog 2 (Drosophila) (SPRY2), mRNA”,
gi|22209007|ref|NM_005842.2|[22209007]; 1031: NM_005845, “Homo sapiens ATP-binding
cassette, sub-family C (CFTR/MRP), member 4 (ABCC4),”, mRNA,
gi|34452699|ref|NM_005845.2|[34452699]; 1032: NM_005850, “Homo sapiens splicing factor
3b, subunit 4, 49 kDa (SF3B4), mRNA”, gi|23111059|ref|NM_005850.3|[23111059]; 1033:
NM_005851, “Homo sapiens tumor suppressor deleted in oral cancer-related 1 (DOC-1R),
mRNA”, gi|39725675|ref|NM_005851.3|[39725675]; 1034: NM_005853, “Homo sapiens
iroquois homeobox protein 5 (IRX5), mRNA”, gi|42415493|ref|NM_005853.3|[42415493];
1035: NM_005856, “Homo sapiens receptor (calcitonin) activity modifying protein 3 (RAMP3),
mRNA”, gi|5032022|ref|NM_005856.1|[5032022]; 1036: NM_005857, “Homo sapiens zinc
metalloproteinase (STE24 homolog, yeast) (ZMPSTE24), mRNA”,
gi|18379365|ref|NM_005857.2|[18379365]; 1037: NM_005860, “Homo sapiens follistatin-like 3
(secreted glycoprotein) (FSTL3), mRNA”, gi|5031700|ref|NM_005860.1|[5031700]; 1038:
NM_005861, “Homo sapiens STIP1 homology and U-Box containing protein 1 (STUB1),
mRNA”, gi|5031962|ref|NM_005861.1|[5031962]; 1039: NM_005873, “Homo sapiens regulator
of G-protein signalling 19 (RGS19), mRNA”, gi|5031704|ref|NM_005873.1|[5031704]; 1040:
NM_005876, “Homo sapiens aortic preferentially expressed protein 1 (APEG1), mRNA”,
gi|37577150|ref|NM_005876.3|[37577150]; 1041: NM_005879, “Homo sapiens TRAF
interacting protein (TRIP), mRNA”, gi|40807468|ref|NM_005879.2|[40807468]; 1042:
NM_005881, “Homo sapiens branched chain alpha-ketoacid dehydrogenase kinase (BCKDK),
mRNA”, gi|5031608|ref|NM_005881.1|[5031608]; 1043: NM_005882, “Homo sapiens
macrophage erythroblast attacher (MAEA), mRNA”, gi|9257203|ref|NM_005882.2|[9257203];
1044: NM_005891, Homo sapiens acetyl-Coenzyme A acetyltransferase 2 (acetoacetyl
Coenzyme A, “thiolase) (ACAT2), mRNA”, gi|5174388|ref|NM_005891.1|[5174388]; 1045:
NM_005895, “Homo sapiens golgi autoantigen, golgin subfamily a, 3 (GOLGA3), mRNA”,
gi|30089939|ref|NM_005895.2|[30089939]; 1046: NM_005900, “Homo sapiens MAD, mothers
against decapentaplegic homolog 1 (Drosophila)”, “(MADH1), mRNA”,
gi|5174508|ref|NM_005900.1|[5174508]; 1047: NM_005904, “Homo sapiens MAD, mothers
against decapentaplegic homolog 7 (Drosophila)”, “(MADH7), mRNA”,
gi|5174516|ref|NM_005904.1|[5174516]; 1048: NM_005908, “Homo sapiens mannosidase, beta
A, lysosomal (MANBA), mRNA”, gi|24797157|ref|NM_005908.2|[24797157]; 1049:
NM_005909, “Homo sapiens microtubule-associated protein 1B (MAP1B), transcript variant
1,”, mRNA, gi|14165457|ref|NM_005909.2|[14165457]; 1050: NM_005912 “Homo sapiens
melanocortin 4 receptor (MC4R), mRNA”, gi|5174532|ref|NM_005912.1|[5174532]; 1051:
NM_005915, “Homo sapiens MCM6 minichromosome maintenance deficient 6 (MIS5
homolog, S.”, “pombe) (S. cerevisiae) (MCM6), mRNA”,
gi|33469920|ref|NM_005915.4|[33469920]; 1052: NM_005917, “Homo sapiens malate
dehydrogenase 1, NAD (soluble) (MDH1), mRNA”, gi|21735619|ref|NM_005917.2|[21735619];
1053: NM_005953, “Homo sapiens metallothionein 2A (MT2A), mRNA”,
gi|31543214|ref|NM_005953.2|[31543214]; 1054: NM_005956, “Homo sapiens
methylenetetrahydrofolate dehydrogenase (NADP+ dependent),”, “methenyltetrahydrofolate
cyclohydrolase, formyltetrahydrofolate synthetase”, “(MTHFD1), mRNA”,
gi|13699867|ref|NM_005956.2|[13699867]; 1055: NM_005958, “Homo sapiens melatonin
receptor 1A (MTNR1A), mRNA”, gi|14141171|ref|NM_005958.2|[14141171]; 1056:
NM_005965, “Homo sapiens myosin, light polypeptide kinase (MYLK), transcript variant 6,
mRNA”, gi|16950600|ref|NM_005965.2|[16950600]; 1057: NM_005975, “Homo sapiens PTK6
protein tyrosine kinase 6 (PTK6), mRNA”, gi|27886594|ref|NM_005975.2|[27886594]; 1058:
NM_005984, Homo sapiens solute carrier family 25 (mitochondrial carrier; citrate,
“transporter), member 1 (SLC25A1), mRNA”, gi|21389314|ref|NM_005984.1|[21389314]; 1059:
NM_005985, “Homo sapiens snail homolog 1 (Drosophila) (SNAI1), mRNA”,
gi|18765740|ref|NM_005985.2|[18765740]; 1060: NM_005996, “Homo sapiens T-box 3 (ulnar
mammary syndrome) (TBX3), transcript variant 1, mRNA”,
gi|18375606|ref|NM_005996.2|[18375606]; 1061: NM_005997, “Homo sapiens transcription
factor-like 1 (TCFL1), mRNA”, gi|5174714|ref|NM_005997.1|[5174714]; 1062: NM_006002,
Homo sapiens ubiquitin carboxyl-terminal esterase L3 (ubiquitin thiolesterase), “(UCHL3),
mRNA”, gi|37059734|ref|NM_006002.3|[37059734]; 1063: NM_006003, “Homo sapiens
ubiquinol-cytochrome c reductase, Rieske iron-sulfur polypeptide 1”, “(UQCRFS1), mRNA”,
gi|5174742|ref|NM_006003.1|[5174742]; 1064: NM_006004, “Homo sapiens ubiquinol-
cytochrome c reductase hinge protein (UQCRH, mRNA”,
gi|5174744|ref|NM_006004.1|[5174744]; 1065: NM_006010, “Homo sapiens arginine-rich,
mutated in early stage tumors (ARMET), mRNA”, gi|5174392|ref|NM_006010.1|[5174392];
1066: NM_006011, “Homo sapiens sialyltransferase 8B (alpha-2,8-sialyltransferase) (SIAT8B),
mRNA”, gi|28373096|ref|NM_006011.21|[28373096]; 1067: NM_006012, “Homo sapiens ClpP
caseinolytic protease, ATP-dependent, proteolytic subunit”, “homolog (E. coli) (CLPP), nuclear
gene encoding mitochondrial protein, mRNA”, gi|5174418|ref|NM_006012.1|[5174418]; 1068:
NM_006017, “Homo sapiens prominin 1 (PROM1), mRNA”,
gi|5174386|ref|NM_006017.1|[5174386]; 1069: NM_006020, “Homo sapiens alkB, alkylation
repair homolog (E. coli) (ALKBH), mRNA”, gi|5174384|ref|NM_006020.1|[5174384]; 1070:
NM_006023, “Homo sapiens chromosome 10 open reading frame 7 (C10orf7), mRNA”,
gi|5174422|ref|NM_006023.1|[5174422]; 1071: NM_006035, “Homo sapiens CDC42 binding
protein kinase beta (DMPK-like) (CDC42BPB), mRNA”,
gi|16357473|ref|NM_006035.2|[16357473]; 1072: NM_006037, “Homo sapiens histone
deacetylase 4 (HDAC4), mRNA”, gi|13259519|ref|NM_006037.2|[13259519]; 1073:
NM_006041, “Homo sapiens heparan sulfate (glucosamine) 3-O-sulfotransferase 3B1
(HS3ST3B1),”, mRNA, gi|5174466|ref|NM_006041.1|[5174466]; 1074: NM_006042, “Homo
sapiens heparan sulfate (glucosamine) 3-O-sulfotransferase 3A1 (HS3ST3A1),”, mRNA,
gi|5174464|ref|NM_006042.1|[5174464]; 1075: NM_006056, “Homo sapiens G protein-coupled
receptor 66 (GPR66), mRNA”, gi|24432088|ref|NM_006056.2|[24432088]; 1076: NM_006061,
“Homo sapiens cysteine-rich secretory protein 3 (CRISP3), mRNA”,
gi|5174674|ref|NM_006061.1|[5174674]; 1077: NM_006067, “Homo sapiens neighbor of COX4
(NOC4), mRNA”, gi|34147520|ret|NM_006067.3|[34147520]; 1078: NM_006070, “Homo
sapiens TRK-fused gene (TFG), mRNA”, gi|34147663|ref|NM_006070.3|[34147663]; 1079:
NM_006080, “Homo sapiens sema domain, immunoglobulin domain (Ig), short basic domain,”,
“secreted, (semaphorin) 3A (SEMA3A), mRNA”, gi|5174672|ref|NM_006080.1|[5174672];
1080: NM_006084, “Homo sapiens interferon-stimulated transcription factor 3, gamma 48 kDa
(ISGF3G),”, mRNA, gi|25282406|ref|NM_006084.3|[25282406]; 1081: NM_006089, “Homo
sapiens sex comb on midleg-like 2 (Drosophila) (SCML2), mRNA”,
gi|5174668|ref|NM_006089.1|[5174668]; 1082: NM_006090, “Homo sapiens
choline/ethanolaminephosphotransferase (CEPT1), mRNA”,
gi|21735567|ref|NM_006090.2|[21735567]; 1083: NM_006091, “Homo sapiens coronin, actin
binding protein, 2B (CORO2B), mRNA”, gi|24307902|ref|NM_006091.1|[24307902]; 1084:
NM_006094, “Homo sapiens deleted in liver cancer 1 (DLC1), transcript variant 2, mRNA”,
gi|33188436|ref|NM_006094.3|[33188436]; 1085: NM_006096, “Homo sapiens N-myc
downstream regulated gene 1 (NDRG1), mRNA”, gi|37655182|ref|NM_006096.2|[37655182];
1086: NM_006097, “Homo sapiens myosin, light polypeptide 9, regulatory (MYL9), transcript
variant”, “1, mRNA”, gi|31563522|ref|NM_006097.3|[31563522]; 1087: NM_006101, “Homo
sapiens kinetochore associated 2 (KNTC2), mRNA”, gi|5174456|ref|NM_006101.1|[5174456];
1088: NM_006103, “Homo sapiens WAP four-disulfide core domain 2 (WFDC2), transcript
variant 1,”, mRNA, gi|18379363|ref|NM_006103.2|[18379363]; 1089: NM_006114, Homo
sapiens translocase of outer mitochondrial membrane 40 homolog (yeast), “(TOMM40),
mRNA”, gi|5174722|ref|NM_006114.1|[5174722]; 1090: NM_006119, “Homo sapiens
fibroblast growth factor 8 (androgen-induced) (FGF8), transcript”, “variant B, mRNA”,
gi|15147351|ref|NM_006119.2|[15147351]; 1091: NM_006122, “Homo
sapiens mannosidase,
alpha, class 2A, member 2 (MAN2A2), mRNA”, gi|5540099|ref|NM_006122.1|[5540099]; 1092:
NM_006133, “Homo sapiens chromosome 11 open reading frame 11 (C11orf11), mRNA”,
gi|27262631|ref|NM_006133.1|[27262631]; 1093: NM_006135, “Homo sapiens capping protein
(actin filament) muscle Z-line, alpha 1 (CAPZA1),”, mRNA,
gi|5453596|ref|NM_006135.1|[5453596]; 1094: NM_006148, “Homo sapiens LIM and SH3
protein 1 (LASP1), mRNA”, gi|5453709|ref|NM_006148.1|[5453709]; 1095: NM_006156,
“Homo sapiens neural precursor cell expressed, developmentally down-regulated 8”, “(NEDD8),
mRNA”, gi|5453759|ref|NM_006156.1|[5453759]; 1096: NM_006157, “Homo sapiens NEL-
like 1 (chicken) (NELL1), mRNA”, gi|5453763|ref|NM_006157.1|[5453763]; 1097: NM_006164
“Homo sapiens nuclear factor (erythroid-derived 2)-like 2 (NFE2L2), mRNA”,
gi|20149575|ref|NM_006164.2|[20149575]; 1098: NM_006168, “Homo sapiens NK6
transcription factor related, locus 1 (Drosophila) (NKX6-1),”, mRNA,
gi|5453787|ref|NM_006168.1|[5453787]; 1099: NM_006172, “Homo sapiens natriuretic peptide
precursor A (NPPA), mRNA”, gi|23510318|ref|NM_006172.1|[23510318]; 1100: NM_006181,
“Homo sapiens netrin 2-like (chicken) (NTN2L), mRNA”,
gi|5453809|ref|NM_006181.1|[5453809]; 1101: NM_006194, “Homo sapiens paired box gene 9
(PAX9), mRNA”, gi|7242166|ref|NM_006194.1|[7242166]; 1102: NM_006195, “Homo sapiens
pre-B-cell leukemia transcription factor 3 (PBX3), mRNA”,
gi|24475894|ref|NM_006195.2|[24475894]; 1103: NM_006196, “Homo sapiens poly(rC)
binding protein 1 (PCBP1), mRNA”, gi|14141164|ref|NM_006196.2|[14141164]; 1104:
NM_006204, “Homo sapiens phosphodiesterase 6C, cGMP-specific, cone, alpha prime
(PDE6C),”, mRNA, gi|21361307|ref|NM_006204.2|[21361307]; 1105: NM_006205, “Homo
sapiens phosphodiesterase 6H, cGMP-specific, cone, gamma (PDE6H), mRNA”,
gi|5453867|ref|NM_006205.1|[5453867]; 1106: NM_006221, Homo sapiens protein (peptidyl-
prolyl cis/trans isomerase) NIMA-interacting 1, “(PIN1), mRNA”,
gi|5453897|ref|NM_006221.1|[5453897]; 1107: NM_006228, “Homo sapiens prepronociceptin
(PNOC), mRNA”, gi|11079650|ref|NM_006228.2|[11079650]; 1108: NM_006232, “Homo
sapiens polymerase (RNA) II (DNA directed) polypeptide H (POLR2H), mRNA”,
gi|14589952|ref|NM_006232.2|[14589952]; 1109: NM_006236, “Homo sapiens POU domain,
class 3, transcription factor 3 (POU3F3), mRNA”, gi|5453935|ref|NM_006236.1|[5453935];
1110: NM_006240, “Homo sapiens protein phosphatase, EF hand calcium-binding domain 1
(PPEF1),”, “transcript variant 1, mRNA”, gi|23312379|ref|NM_006240.2|[23312379]; 1111:
NM_006246, “Homo sapiens protein phosphatase 2, regulatory subunit B (B56), epsilon
isoform”, “(PPP2R5E), mRNA”, gi|31083295|ref|NM_006246.2|[31083295]; 1112: NM_006254
“Homo sapiens protein kinase C, delta (PRKCD), mRNA”,
gi|31377781|ref|NM_006254.2|[31377781]; 1113: NM_006259, “Homo sapiens protein kinase,
cGMP-dependent, type II (PRKG2), mRNA”, gi|5453977|ref|NM_006259.1|[5453977]; 1114:
NM_006261, “Homo sapiens prophet of Pit1, paired-like homeodomain transcription factor”,
“(PROP1), mRNA”, gi|40254838|ref|NM_006261.2|[40254838]; 1115: NM_006262, “Homo
sapiens peripherin (PRPH), mRNA”, gi|21264344|ref|NM_006262.2|[21264344]; 1116:
NM_006263, “Homo sapiens proteasome (prosome, macropain) activator subunit 1 (PA28
alpha)”, “(PSME1), transcript variant 1, mRNA”, gi|30581139|ref|NM_006263.2|[30581139];
1117: NM_006270, “Homo sapiens related RAS viral (r-ras) oncogene homolog (RRAS),
mRNA”, gi|20127497|ref|NM_006270.2|[20127497]; 1118: NM_006280, “Homo sapiens signal
sequence receptor, delta (translocon-associated protein”, “delta) (SSR4), mRNA”,
gi|5454089|ref|NM_006280.1|[5454089]; 1119: NM_006284, “Homo sapiens TAF10 RNA
polymerase II, TATA box binding protein (TBP)-associated”, “factor, 30 kDa (TAF10), mRNA”,
gi|21166374|ref|NM_006284.2|[21166374]; 1120: NM_006285, “Homo sapiens testis-specific
kinase 1 (TESK1), mRNA”, gi|5454109|ref|NM_006285.1|[5454109]; 1121: NM_006289,
“Homo sapiens talin 1 (TLN1), mRNA”, gi|16753232|ref|NM_006289.2|[16753232]; 1122:
NM_006292, “Homo sapiens tumor susceptibility gene 101 (TSG101), mRNA”,
gi|18765712|ref|NM_006292.2|[18765712]; 1123: NM_006298, “Homo sapiens zinc finger
protein 192 (ZNF192), mRNA”, gi|5454177|ref|NM_006298.1|[5454177]; 1124: NM_006302,
“Homo sapiens glucosidase I (GCS1), mRNA”, gi|5453661|ref|NM_006302.1|[5453661]; 1125:
NM_006315, “Homo sapiens ring finger protein 3 (RNF3), mRNA”,
gi|34305288|ref|NM_006315.3|[34305288]; 1126: NM_006329, “Homo sapiens fibulin 5
(FBLN5), mRNA”, gi|19743802|ref|NM_006329.2|[19743802]; 1127: NM_006331, “Homo
sapiens C2f protein (C2F), mRNA”, gi|31652261|ref|NM_006331.3|[31652261]; 1128:
NM_006333, “Homo sapiens nuclear DNA-binding protein (C1D), transcript variant 1, mRNA”,
gi|27894371|ref|NM_006333.2|[27894371]; 1129: NM_006338, “Homo sapiens leucine rich
repeat neuronal 5 (LRRN5), transcript variant 1, mRNA”,
gi|42544230|ref|NM_006338.2|[42544230]; 1130: NM_006342, “Homo sapiens transforming,
acidic coiled-coil containing protein 3 (TACC3), mRNA”,
gi|5454101|ref|NM_006342.1|[5454101]; 1131: NM_006344, “Homo sapiens C-type (calcium
dependent, carbohydrate-recognition domain) lectin,”, “superfamily member 13 (macrophage-
derived) (CLECSF13), transcript variant 2,”, mRNA, gi|5453683|ref|NM_006344.1|[5453683];
1132: NM_006345, “Homo sapiens solute carrier family 30 (zinc transporter), member 9
(SLC30A9),”, mRNA, gi|7656945|ref|NM_006345.2|[7656945]; 1133: NM_006346, “Homo
sapiens progesterone-induced blocking factor 1 (PIBF1), mRNA”,
gi|5453889|ref|NM_006346.1|[5453889]; 1134: NM_006347, “Homo sapiens peptidyl prolyl
isomerase H (cyclophilin H) (PPIH), mRNA”, gi|19224661|ref|NM_006347.2|[19224661]; 1135:
NM_006356, “Homo sapiens ATP synthase, H+ transporting, mitochondrial F0 complex,
subunit d”, “(ATP5H), mRNA”, gi|5453558|ref|NM_006356.1|[5453558]; 1136: NM_006357,
“Homo sapiens ubiquitin-conjugating enzyme E2E 3 (UBC4/5 homolog, yeast)”, “(UBE2E3),
transcript variant 1, mRNA”, gi|33359695|ref|NM_006357.2|[33359695]; 1137: NM_006365,
“Homo sapiens transcriptional activator of the c-fos promoter (CROC4), mRNA”,
gi|5453624|ref|NM_006365.1|[5453624]; 1138: NM_006368, “Homo sapiens cAMP responsive
element binding protein 3 (CREB3), mRNA”, gi|38327637|ref|NM_006368.4|[38327637]; 1139:
NM_006370, Homo sapiens vesicle transport through interaction with t-SNAREs homolog 1B,
“(yeast) (VTI1B), mRNA”, gi|5454165|ref|NM_006370.1|[5454165]; 1140: NM_006374,
“Homo sapiens serine/threonine kinase 25 (STE20 homolog, yeast) (STK25), mRNA”,
gi|34147665|ref|NM_006374.3|[34147665]; 1141: NM_006389, “Homo sapiens hypoxia up-
regulated 1 (HYOU1), mRNA”, gi|13699861|ref|NM_006389.2|[13699861]; 1142: NM_006390,
“Homo sapiens importin 8 (IPO8), mRNA”, gi|5453999|ref|NM_006390.1|[5453999]; 1143:
NM_006395, “Homo sapiens APG7 autophagy 7-like (S. cerevisiae) (APG7L), mRNA”,
gi|5453667|ref|NM_006395.1|[5453667]; 1144: NM_006396, “Homo sapiens Sjogren's
syndrome/scleroderma autoantigen 1 (SSSCA1), mRNA”,
gi|5453837|ref|NM_006396.1|[5453837]; 1145: NM_006397, “Homo sapiens ribonuclease H2,
large subunit (RNASEH2A), mRNA”, gi|38455390|ref|NM_006397.2|[38455390]; 1146:
NM_006399, “Homo sapiens basic leucine zipper transcription factor, ATF-like (BATF),
mRNA”, gi|18375640|ref|NM_006399.2|[18375640]; 1147: NM_006408, “Homo sapiens
anterior gradient 2 homolog (Xenopus laevis) (AGR2), mRNA”,
gi|20070225|ref|NM_006408.2|[20070225]; 1148: NM_006422, “Homo sapiens A kinase
(PRKA) anchor protein 3 (AKAP3), mRNA”, gi|21493040|ref|NM_006422.2|[21493040]; 1149:
NM_006428, “Homo sapiens mitochondrial ribosomal protein L28 (MRPL28), nuclear gene
encoding”, “mitochondrial protein, mRNA”, gi|39812062|ref|NM_006428.3|[39812062]; 1150:
NM_006447, “Homo sapiens ubiquitin specific protease 16 (USP16), mRNA”,
gi|5454155|ref|NM_006447.1|[5454155]; 1151: NM_006453, “Homo sapiens transducin (beta)-
like 3 (TBL3), mRNA”, gi|19913368|ref|NM_006453.2|[19913368]; 1152: NM_006455, “Homo
sapiens synaptonemal complex protein SC65 (SC65), mRNA”,
gi|39812427|ref|NM_006455.2|[39812427]; 1153: NM_006465, “Homo sapiens AT rich
interactive domain 3B (BRIGHT-like) (ARID3B), mRNA”,
gi|5453637|ref|NM_006465.1|[5453637]; 1154: NM_006467, “Homo sapiens polymerase
(RNA) III (DNA directed) (32 kD) (RPC32), mRNA”, gi|5454017|ref|NM_006467.1|[5454017];
1155: NM_006477, “Homo sapiens RAS-related on chromosome 22 (RRP22), mRNA”,
gi|42476128|ref|NM_006477.2|[42476128]; 1156: NM_006479, “Homo sapiens RAD51-
interacting protein (PIR51), mRNA”, gi|19923778|ref|NM_006479.2|[19923778]; 1157:
NM_006492, “Homo sapiens aristaless-like homeobox 3 (ALX3), mRNA”,
gi|5729727|ref|NM_006492.1|[5729727]; 1158: NM_006495, “Homo sapiens ecotropic viral
integration site 2B (EVI2B), mRNA”, gi|20070234|ref|NM_006495.2|[20070234]; 1159:
NM_006497, “Homo sapiens hypermethylated in cancer 1 (HIC1), mRNA”,
gi|5729870|ref|NM_006497.1|[5729870]; 1160: NM_006502, “Homo sapiens polymerase (DNA
directed), eta (POLH), mRNA”, gi|5729981|ref|NM_006502.1|[5729981]; 1161: NM_006503,
“Homo sapiens proteasome (prosome, macropain) 26S subunit, ATPase, 4 (PSMC4),”,
“transcript variant 1, mRNA”, gi|24430156|ref|NM_006503.2|[24430156]; 1162: NM_006513,
“Homo sapiens seryl-tRNA synthetase (SARS), mRNA”,
gi|16306547|ref|NM_006513.2|[16306547]; 1163: NM_006515, “Homo sapiens SET domain
and mariner transposase fusion gene (SETMAR), mRNA”,
gi|5730038|ref|NM_006515.1|[5730038]; 1164: NM_006530, “Homo sapiens glioma-amplified
sequence-41 (GAS41), mRNA”, gi|29337287|ref|NM_006530.2|[29337287]; 1165: NM_006531
“Homo sapiens Probe hTg737 (polycystic kidney disease, autosomal recessive)”, “(TG737),
transcript variant 2, mRNA”, gi|28329438|ref|NM_006531.2|[28329438]; 1166: NM_006537,
“Homo sapiens ubiquitin specific protease 3 (USP3), mRNA”,
gi|5730109|ref|NM_006537.1|[5730109]; 1167: NM_006538, “Homo sapiens BCL2-like 11
(apoptosis facilitator) (BCL2L11), transcript variant”, “6, mRNA”,
gi|5729739|ref|NM_006538.1|[5729739]; 1168: NM_006539, “Homo sapiens calcium channel,
voltage-dependent, gamma subunit 3 (CACNG3), mRNA”,
gi|22027545|ref|NM_006539.2|[22027545]; 1169: NM_006548, “Homo sapiens IGF-II mRNA-
binding protein 2 (IMP-2), mRNA”, gi|34222220|ref|NM_006548.3|[34222220]; 1170:
NM_006554, “Homo sapiens metaxin 2 (MTX2), mRNA”,
gi|5729936|ref|NM_006554.1|[5729936]; 1171: NM_006556, “Homo sapiens
phosphomevalonate kinase (PMVK), mRNA”, gi|20127505|ref|NM_006556.2|[20127505]; 1172:
NM_006570, “Homo sapiens Ras-related GTP binding A (RRAGA), mRNA”,
gi|34147579|ref|NM_006570.3|[34147579]; 1173: NM_006577, “Homo sapiens UDP-
GlcNAc:betaGal beta-1,3-N-acetylglucosaminyltransferase 1”, “(B3GNT1), transcript variant 1,
mRNA”, gi|15451893|ref|NM_006577.3|[15451893]; 1174: NM_006582, “Homo sapiens
glucocorticoid modulatory element binding protein 1 (GMEB1),”, “transcript variant 1, mRNA”,
gi|13435376|ref|NM_006582.2|[13435376]; 1175: NM_006584, “Homo sapiens chaperonin
containing TCP1, subunit 6B (zeta 2) (CCT6B), mRNA”,
gi|5729760|ref|NM_006584.1|[5729760]; 1176: NM_006585,,
ref|NM_006585.1|CCT8[6005726], This record was temporarily removed by RefSeq staff for
additional review.,, 1177: NM_006586, “Homo sapiens trinucleotide repeat containing 5
(TNRC5), mRNA”, gi|33942071|ref|NM_006586.2|[33942071]; 1178: NM_006589, “Homo
sapiens chromosome 1 open reading frame 2 (C1orf2), transcript variant 1,”, mRNA,
gi|38146115|ref|NM_006589.2|[38146115]; 1179: NM_006593, “Homo sapiens T-box, brain, 1
(TBR1), mRNA”, gi|22547231|ref|NM_006593.2|[22547231]; 1180: NM_006604, “Homo
sapiens ret finger protein-like 3 (RFPL3), mRNA”, gi|5730012|ref|NM_006604.1|[5730012];
1181: NM_006611, “Homo sapiens killer cell lectin-like receptor subfamily A, member 1
(KLRA1),”, mRNA, gi|5729898|ref|NM_006611.1|[5729898]; 1182: NM_006622, “Homo
sapiens polo-like kinase 2 (Drosophila) (PLK2), mRNA”,
gi|5730054|ref|NM_006622.1|[5730054]; 1183: NM_006626, “Homo sapiens zinc finger protein
482 (ZNF482), mRNA”, gi|34222260|ref|NM_006626.3|[34222260]; 1184: NM_006627,
“Homo sapiens POP4 (processing of precursor, S. cerevisiae) homolog (POP4), mRNA”,
gi|5729985|ref|NM_006627.1|[5729985]; 1185: NM_006631, “Homo sapiens zinc finger protein
266 (ZNF266), mRNA”, gi|37622348|ref|NM_006631.2|[37622348]; 1186: NM_006633,
“Homo sapiens IQ motif containing GTPase activating protein 2 (IQGAP2), mRNA”,
gi|5729886|ref|NM_006633.1|[5729886]; 1187: NM_006638, “Homo sapiens ribonuclease P1
(RNASEP1), mRNA”, gi|5730016|ref|NM_006638.1|[5730016]; 1188: NM_006642, “Homo
sapiens serologically defined colon cancer antigen 8 (SDCCAG8), mRNA”,
gi|28269671|ref|NM_006642.1|[28269671]; 1189: NM_006654, “Homo sapiens fibroblast
growth factor receptor substrate 2 (FRS2), mRNA”, gi|21314643|ref|NM_006654.2|[21314643];
1190: NM_006664, “Homo sapiens chemokine (C—C motif) ligand 27 (CCL27), mRNA”,
gi|22165428|ref|NM_006664.2|[22165428]; 1191: NM_006666, “Homo sapiens RuvB-like 2 (E. coli)
(RUVBL2), mRNA”, gi|5730022|ref|NM_006666.1|[5730022]; 1192: NM_006670, “Homo
sapiens trophoblast glycoprotein (TPBG), mRNA”, gi|34222307|ref|NM_006670.3|[34222307];
1193: NM_006675, “Homo sapiens transmembrane 4 superfamily member tetraspan NET-5
(NET-5), mRNA”, gi|21264572|ref|NM_006675.2|[21264572]; 1194: NM_006697, “Homo
sapiens cisplatin resistance associated (CRA), mRNA”, gi|5870890|ref|NM_006697.1|[5870890];
1195: NM_006698, “Homo sapiens bladder cancer associated protein (BLCAP), mRNA”,
gi|5729737|ref|NM_006698.1|[5729737]; 1196: NM_006702, “Homo sapiens neuropathy target
esterase (NTE), mRNA”, gi|31543298|ref|NM_006702.2|[31543298]; 1197: NM_006715,
“Homo sapiens mannosidase, alpha, class 2C, member 1 (MAN2C1), mRNA”,
gi|6631092|ref|NM_006715.1|[6631092]; 1198: NM_006730, “Homo sapiens deoxyribonuclease
I-like 1 (DNASE1L1), mRNA”, gi|5803006|ref|NM_006730.1|[5803006]; 1199: NM_006735,
“Homo sapiens homeo box A2 (HOXA2), mRNA”, gi|37596298|ref|NM_006735.3|[37596298];
1200: NM_006736, “Homo sapiens DnaJ (Hsp40) homolog, subfamily B, member 2 (DNAJB2),
mRNA”, gi|34222304|ref|NM_006736.4|[34222304]; 1201: NM_006744, “Homo sapiens retinol
binding protein 4, plasma (RBP4), mRNA”, gi|8400727|ref|NM_006744.2|[8400727]; 1202:
NM_006745, “Homo sapiens sterol-C4-methyl oxidase-like (SC4MOL), mRNA”,
gi|9257238|ref|NM_006745.2|[9257238]; 1203: NM_006747, “Homo sapiens signal-induced
proliferation-associated gene 1 (SIPA1), transcript”, “variant 2, mRNA”,
gi|24497626|ref|NM_006747.2|[24497626]; 1204: NM_006749, “Homo sapiens solute carrier
family 20 (phosphate transporter), member 2”, “(SLC20A2), mRNA”,
gi|34222154|ref|NM_006749.3|[34222154]; 1205: NM_006751, “Homo sapiens sperm specific
antigen 2 (SSFA2), mRNA”, gi|34222128|ref|NM_006751.3|[34222128]; 1206: NM_006756,
“Homo sapiens transcription elongation factor A (SII), 1 (TCEA1), mRNA”,
gi|5803190|ref|NM_006756.1|[5803190]; 1207: NM_006759, “Homo sapiens UDP-glucose
pyrophosphorylase 2 (UGP2), mRNA”, gi|13027637|ref|NM_006759.2|[13027637]; 1208:
NM_006764, “Homo sapiens interferon-related developmental regulator 2 (IFRD2), mRNA”,
gi|21361365|ref|NM_006764.2|[21361365]; 1209: NM_006777, “Homo sapiens kaiso (ZNF-
kaiso), mRNA”, gi|41152068|ref|NM_006777.3|[41152068]; 1210: NM_006784, “Homo sapiens
WD repeat domain 3 (WDR3), mRNA”, gi|5803220|ref|NM_006784.1|[5803220]; 1211:
NM_006793, “Homo sapiens peroxiredoxin 3 (PRDX3), nuclear gene encoding mitochondrial”,
“protein, transcript variant 1, mRNA”, gi|32483378|ref|NM_006793.2|[32483378]; 1212:
NM_006801, Homo sapiens KDEL (Lys-Asp-Glu-Leu) endoplasmic reticulum protein
retention, “receptor 1 (KDELR1), mRNA”, gi|32307173|ref|NM_006801.2|[32307173]; 1213:
NM_006802, “Homo sapiens splicing factor 3a, subunit 3, 60 kDa (SF3A3), mRNA”,
gi|5803166|ref|NM_006802.1|[5803166]; 1214: NM_006804, “Homo sapiens START domain
containing 3 (STARD3), mRNA”, gi|31543656|ref|NM_006804.2|[31543656]; 1215:
NM_006809, “Homo sapiens translocase of outer mitochondrial membrane 34 (TOMM34),
mRNA”, gi|40807467|ref|NM_006809.4|[40807467]; 1216: NM_006813, “Homo sapiens
proline-rich nuclear receptor coactivator 1 (PNRC1), mRNA”,
gi|5802981|ref|NM_006813.1|[5802981]; 1217: NM_006816, “Homo sapiens lectin, mannose-
binding 2 (LMAN2), mRNA”, gi|5803022|ref|NM_006816.1|[5803022]; 1218: NM_006817,
“Homo sapiens chromosome 12 open reading frame 8 (C12orf8), mRNA”,
gi|13124889|ref|NM_006817.2|[13124889]; 1219: NM_006818, “Homo sapiens ALL1-fused
gene from chromosome 1q (AF1Q), mRNA”, gi|21626459|ref|NM_006818.2|[21626459]; 1220:
NM_006824, “Homo sapiens EBNA1 binding protein 2 (EBNA1BP2), mRNA”,
gi|5803110|ref|NM_006824.1|[5803110]; 1221: NM_006828, “Homo sapiens helicase, ATP
binding 1 (HELIC1), mRNA”, gi|24307916|ref|NM_006828.1|[24307916]; 1222: NM_006830,
“Homo sapiens ubiquinol-cytochrome c reductase (6.4 kD) subunit (UQCR), mRNA”,
gi|19923785|ref|NM_006830.2|[19923785]; 1223: NM_006831, “Homo sapiens ATP/GTP-
binding protein (HEAB), mRNA”, gi|5803028|ref|NM_006831.1|[5803028]; 1224: NM_006837
Homo sapiens COP9 constitutive photomorphogenic homolog subunit 5 (Arabidopsis),
“(COPS5), mRNA”, gi|38027922|ref|NM_006837.2|[38027922]; 1225: NM_006839, “Homo
sapiens inner membrane protein, mitochondrial (mitofilin) (IMMT), mRNA”,
gi|5803114|ref|NM_006839.1|[5803114]; 1226: NM_006841, “Homo sapiens solute carrier
family 38, member 3 (SLC38A3), mRNA”, gi|40795668|ref|NM_006841.3|[40795668]; 1227:
NM_006843, “Homo sapiens serine dehydratase (SDS), mRNA”,
gi|33469957|ref|NM_006843.2|[33469957]; 1228: NM_006876, “Homo sapiens UDP-
GlcNAc: betaGal beta-1,3-N-acetylglucosaminyltransferase 6”, “(B3GNT6), mRNA”,
gi|5802983|ref|NM_006876.1|[5802983]; 1229: NM_006886, “Homo sapiens ATP synthase, H+
transporting, mitochondrial F1 complex, epsilon”, “subunit (ATP5E), nuclear gene encoding
mitochondrial protein, mRNA”, gi|21327678|ref|NM_006886.2|[21327678]; 1230: NM_006901,
“Homo sapiens myosin IXA (MYO9A), mRNA”, gi|5902011|ref|NM_006901.1|[5902011];
1231: NM_006913, “Homo sapiens ring finger protein 5 (RNF5), mRNA”,
gi|34305290|ref|NM_006913.2|[34305290]; 1232: NM_006917, “Homo sapiens retinoid X
receptor, gamma (RXRG), mRNA”, gi|21361386|ref|NM_006917.2|[21361386]; 1233:
NM_006923, “Homo sapiens stromal cell-derived factor 2 (SDF2), mRNA”,
gi|14141194|ref|NM_006923.2|[14141194]; 1234: NM_006928, “Homo sapiens silver homolog
(mouse) (SILV), mRNA”, gi|42542384|ref|NM_006928.3|[42542384]; 1235: NM_006929,
“Homo sapiens superkiller viralicidic activity 2-like (S. cerevisiae) (SKIV2L),”, mRNA,
gi|20631986|ref|NM_006929.3|[20631986]; 1236: NM_006934, “Homo sapiens solute carrier
family 6 (neurotransmitter transporter, glycine),”, “member 9 (SLC6A9), transcript variant 1,
mRNA”, gi|5902093|ref|NM_006934.1|[5902093]; 1237: NM_006946, “Homo sapiens spectrin,
beta, non-erythrocytic 2 (SPTBN2), mRNA”, gi|5902121|ref|NM_006946.1|[5902121]; 1238:
NM_006949, “Homo sapiens syntaxin binding protein 2 (STXBP2), mRNA”,
gi|5902127|ref|NM_006949.1|[5902127]; 1239: NM_006950, “Homo sapiens synapsin I
(SYN1), transcript variant Ia, mRNA”, gi|19924098|ref|NM_006950.2|[19924098]; 1240:
NM_006973, “Homo sapiens zinc finger protein 32 (KOX 30) (ZNF32), mRNA”,
gi|24307924|ref|NM_006973.1|[24307924]; 1241: NM_006977, “Homo sapiens zinc finger
protein 46 (KUP) (ZNF46), mRNA”, gi|40217848|ref|NM_006977.2|[40217848]; 1242:
NM_006979, “Homo sapiens solute carrier family 39 (zinc transporter), member 7
(SLC39A7),”, mRNA, gi|5901935|ref|NM_006979.1|[5901935]; 1243: NM_006980, “Homo
sapiens transcription termination factor, mitochondrial (MTERF), nuclear”, “gene encoding
mitochondrial protein, mRNA”, gi|14790134|ref|NM_006980.2|[14790134]; 1244: NM_006982,
“Homo sapiens cartilage paired-class homeoprotein 1 (CART1), mRNA”,
gi|5901917|ref|NM_006982.1|[5901917]; 1245: NM_006984, “Homo sapiens claudin 10
(CLDN10), transcript variant 2, mRNA”, gi|38570070|ref|NM_006984.3|[38570070]; 1246:
NM_006987, “Homo sapiens rabphilin 3A-like (without C2 domains) (RPH3AL), mRNA”,
gi|31543557|ref|NM_006987.2|[31543557]; 1247: NM_006988, Homo sapiens a disintegrin-like
and metalloprotease (reprolysin type) with, “thrombospondin type 1 motif, 1 (ADAMTS1),
mRNA”, gi|11038653|ref|NM_006988.2|[11038653]; 1248: NM_006992, “Homo sapiens B7
gene (B7), transcript variant 2, mRNA”, gi|42542401|ref|NM_006992.2|[42542401]; 1249:
NM_006993, “Homo sapiens nucleophosmin/nucleoplasmin, 3 (NPM3), mRNA”,
gi|6857817|ref|NM_006993.1|[6857817]; 1250: NM_006998, “Homo sapiens secretagogin, EF-
hand calcium binding protein (SCGN), mRNA”, gi|15055536|ref|NM_006998.2|[15055536];
1251: NM_007002, “Homo sapiens adhesion regulating molecule 1 (ADRM1), transcript variant
1, mRNA”, gi|28373191|ref|NM_007002.2|[28373191]; 1252: NM_007006, “Homo sapiens
cleavage and polyadenylation specific factor 5, 25 kDa (CPSF5),”, mRNA,
gi|5901925|ref|NM_007006.1|[5901925]; 1253: NM_007007, “Homo sapiens cleavage and
polyadenylation specific factor 6, 68 kDa (CPSF6), mRNA”,
gi|5901927|ref|NM_007007.1|[5901927]; 1254: NM_007009, “Homo sapiens zona pellucida
binding protein (ZPBP), mRNA”, gi|5902115|ref|NM_007009.1|[5902115]; 1255: NM_007019,
“Homo sapiens ubiquitin-conjugating enzyme E2C (UBE2C), transcript variant 1,”, mRNA,
gi|32967292|ref|NM_007019.2|[32967292]; 1256: NM_007022, “Homo sapiens putative tumor
suppressor 101F6 (101F6), mRNA”, gi|31541779|ref|NM_007022.3|[31541779]; 1257:
NM_007024, “Homo sapiens placental protein 6 (PL6), mRNA”,
gi|40795669|ref|NM_007024.4|[40795669]; 1258: NM_007027, “Homo sapiens topoisomerase
(DNA) II binding protein (TOPBP1), mRNA”, gi|20143948|ref|NM_007027.2|[20143948]; 1259:
NM_007031, “Homo sapiens heat shock transcription factor 2 binding protein (HSF2BP),
mRNA”, gi|5901979|ref|NM_007031.1|[5901979]; 1260: NM_007038, Homo sapiens a
disintegrin-like and metalloprotease (reprolysin type) with, “thrombospondin type 1 motif, 5
(aggrecanase-2) (ADAMTS5), mRNA”, gi|5901887|ref|NM_007038.1|[5901887]; 1261:
NM_007046, “Homo sapiens elastin microfibril interfacer 1 (EMILIN1), mRNA”,
gi|5901943|ref|NM_007046.1|[5901943]; 1262: NM_007050, “Homo sapiens protein tyrosine
phosphatase, receptor type, T (PTPRT), transcript”, “variant 2, mRNA”,
gi|19743928|ref|NM_007050.3|[19743928]; 1263: NM_007051, “Homo sapiens Fas (TNFRSF6)
associated factor 1 (FAF1), transcript variant 1,”, mRNA,
gi|19528653|ref|NM_007051.2|[19528653]; 1264: NM_007056, “Homo sapiens splicing factor,
arginine/serine-rich 16”, “(suppressor-of-white-apricot homolog, Drosophila) (SFRS16),
mRNA”, gi|5902129|ref|NM_007056.1|[5902129]; 1265: NM_007059, “Homo sapiens kaptin
(actin binding protein) (KPTN), mRNA”, gi|5901993|ref|NM_007059.1|[5901993]; 1266:
NM_007064, Homo sapiens serine/threonine kinase with Dbl- and pleckstrin homology
domains, “(TRAD), mRNA”, gi|5902139|ref|NM_007064.1|[5902139]; 1267: NM_007065,
“Homo sapiens CDC37 cell division cycle 37 homolog (S. cerevisiae) (CDC37), mRNA”,
gi|39995072|ref|NM_007065.3|[39995072]; 1268: NM_007066, “Homo sapiens protein kinase
(cAMP-dependent, catalytic) inhibitor gamma (PKIG),”, “transcript variant 2, mRNA”,
gi|32483384|ref|NM_007066.3|[32483384]; 1269: NM_007069, “Homo sapiens HRAS-like
suppressor 3 (HRASLS3), mRNA”, gi|5901975|ref|NM_007069.1|[5901975]; 1270: NM_007072,
“Homo sapiens HERV-H LTR-associating 2 (HHLA2), mRNA”,
gi|31542933|ref|NM_007072.2|[31542933]; 1271: NM_007076,, ref|NM_007076.2|[42794619];
1272: NM_007081, “Homo sapiens RAB, member of RAS oncogene family-like 2B
(RABL2B), m2RNA”, gi|5902039|ref|NM_007081.1|[5902039]; 1273: NM_007082, “Homo
sapiens RAB, member of RAS oncogene family-like 2A (RABL2A), transcript”, “variant 2,
mRNA”, gi|7549818|ref|NM_007082.2|[7549818]; 1274: NM_007083, Homo sapiens nudix
(nucleoside diphosphate linked moiety X)-type motif 6, “(NUDT6), transcript variant 1, mRNA”,
gi|37594465|ref|NM_007083.3|[37594465]; 1275: NM_007107, “Homo sapiens signal sequence
receptor, gamma (translocon-associated protein”, “gamma) (SSR3), mRNA”,
gi|28416942|ref|NM_007107.2|[28416942]; 1276: NM_007114, “Homo sapiens TATA element
modulatory factor 1 (TMF1), mRNA”, gi|6005903|ref|NM_007114.1|[6005903]; 1277:
NM_007117, “Homo sapiens thyrotropin-releasing hormone (TRH), mRNA”,
gi|6005919|ref|NM_007117.1|[6005919]; 1278: NM_007130, “Homo sapiens zinc finger protein
41 (ZNF41), transcript variant 1, mRNA”, gi|23510456|ref|NM_007130.1|[23510456]; 1279:
NM_007136, “Homo sapiens zinc finger protein 80 (pT17) (ZNF80), mRNA”,
gi|6005981|ref|NM_007136.1|[6005981]; 1280: NM_007147, “Homo sapiens zinc finger protein
175 (ZNF175), mRNA”, gi|37594438|ref|NM_007147.2|[37594438]; 1281: NM_007149,
“Homo sapiens zinc finger protein 184 (Kruppel-like) (ZNF184), mRNA”,
gi|24307934|ref|NM_007149.1|[24307934]; 1282: NM_007152, “Homo sapiens zinc finger
protein 195 (ZNF195), mRNA”, gi|6005973|ref|NM_007152.1|[6005973]; 1283: NM_007158,
“Homo sapiens NRAS-related gene (D1S155E), mRNA”,
gi|41282241|ref|NM_007158.3|[41282241]; 1284: NM_007180, “Homo sapiens trehalase
(brush-border membrane glycoprotein) (TREH), mRNA”,
gi|6005913|ref|NM_007180.1|[6005913]; 1285: NM_007191, “Homo sapiens WNT inhibitory
factor 1 (WIF1), mRNA”, gi|18379354|ref|NM_007191.2|[18379354]; 1286: NM_007192,
“Homo sapiens suppressor of Ty 16 homolog (S. cerevisiae) (SUPT16H), mRNA”,
gi|19924176|ref|NM_007192.2|[19924176]; 1287: NM_007195, “Homo sapiens polymerase
(DNA directed) iota (POLI), mRNA”, gi|6005847|ref|NM_007195.1|[6005847]; 1288:
NM_007208, “Homo sapiens mitochondrial ribosomal protein L3 (MRPL3), nuclear gene
encoding”, “mitochondrial protein, mRNA”, gi|21265090|ref|NM_007208.2|[21265090]; 1289:
NM_007211, “Homo sapiens chromosome 12 open reading frame 2 (C12orf2), mRNA”,
gi|23503242|ref|NM_007211.2|[23503242]; 1290: NM_007212, “Homo sapiens ring finger
protein 2 (RNF2), mRNA”, gi|34305287|ref|NM_007212.2|[34305287]; 1291: NM_007215,
“Homo sapiens polymerase (DNA directed), gamma 2, accessory subunit (POLG2), mRNA”,
gi|6005837|ref|NM_007215.1|[6005837]; 1292: NM_007216, “Homo sapiens Hermansky-
Pudlak syndrome 5 (HPS5), transcript variant 2, mRNA”,
gi|31657126|ref|NM_007216.3|[31657126]; 1293: NM_007217, “Homo sapiens programmed
cell death 10 (PDCD10), transcript variant 1, mRNA”,
gi|22538790|ref|NM_007217.3|[22538790]; 1294: NM_007221, “Homo sapiens polyamine-
modulated factor 1 (PMF1), mRNA”, gi|6005831|ref|NM_007221.1|[6005831]; 1295:
NM_007229, Homo sapiens protein kinase C and casein kinase substrate in neurons 2,
“(PACSIN2), mRNA”, gi|6005825|ref|NM_007229.1|[6005825]; 1296: NM_007231, “Homo
sapiens solute carrier family 6 (neurotransmitter transporter), member 14”, “(SLC6A14),
mRNA”, gi|6005714|ref|NM_007231.1|[6005714]; 1297: NM_007234, “Homo sapiens dynactin
3 (p22) (DCTN3), transcript variant 1, mRNA”, gi|22165423|ref|NM_007234.3|[22165423];
1298: NM_007235, “Homo sapiens exportin, tRNA (nuclear export receptor for tRNAs)
(XPOT), mRNA”, gi|40217845|ref|NM_007235.3|[40217845]; 1299: NM_007246, “Homo
sapiens kelch-like 2, Mayven (Drosophila) (KLHL2), mRNA”,
gi|21359895|ref|NM_007246.2|[21359895]; 1300: NM_007252, “Homo sapiens POU domain,
class 6, transcription factor 2 (POU6F2), mRNA”, gi|6005855|ref|NM_007252.1|[6005855];
1301: NM_007254, “Homo sapiens polynucleotide kinase 3′-phosphatase (PNKP), mRNA”,
gi|31543418|ref|NM_007254.2|[31543418]; 1302: NM_007262, “Homo sapiens Parkinson
disease (autosomal recessive, early onset) 7 (PARK7),”, mRNA,
gi|34222306|ref|NM_007262.3|[34222306]; 1303: NM_007263, “Homo sapiens coatomer
protein complex, subunit epsilon (COPE), transcript”, “variant 1, mRNA”,
gi|40805821|ref|NM_007263.3|[40805821]; 1304: NM_007264, “Homo sapiens adrenomedullin
receptor (ADMR), mRNA”, gi|6466448|ref|NM_007264.2|[6466448]; 1305: NM_007265,
“Homo sapiens suppressor of S. cerevisiae gcr2 (HSGT1), mRNA”,
gi|6005783|ref|NM_007265.1|[6005783]; 1306: NM_007270, “Homo sapiens FK506 binding
protein 9, 63 kDa (FKBP9), mRNA”, gi|33469984|ref|NM_007270.2|[33469984]; 1307:
NM_007273, “Homo sapiens repressor of estrogen receptor activity (REA), mRNA”,
gi|31543548|ref|NM_007273.3|[31543548]; 1308: NM_007277, “Homo sapiens SEC6-like 1 (S. cerevisiae)
(SEC6L1), mRNA”, gi|38148698|ref|NM_007277.3|[38148698]; 1309: NM_007278,
“Homo sapiens GABA(A) receptor-associated protein (GABARAP), mRNA”,
gi|6005763|ref|NM_007278.1|[6005763]; 1310: NM_007280, “Homo sapiens Opa-interacting
protein 5 (OIP5), mRNA”, gi|24307928|ref|NM_007280.1|[24307928]; 1311: NM_007285,
“Homo sapiens GABA(A) receptor-associated protein-like 2 (GABARAPL2), mRNA”,
gi|27374999|ref|NM_007285.6|[27374999]; 1312: NM_007353, “Homo sapiens guanine
nucleotide binding protein (G protein) alpha 12 (GNA12),”, mRNA,
gi|42476110|ref|NM_007353.2|[42476110]; 1313: NM_007357, “Homo sapiens component of
oligomeric golgi complex 2 (COG2), mRNA”, gi|6678675|ref|NM_007357.1|[6678675]; 1314:
NM_007364, “Homo sapiens integral type I protein (P24B), mRNA”,
gi|6679188|ref|NM_007364.1|[6679188]; 1315: NM_007365, “Homo sapiens peptidyl arginine
deiminase, type II (PADI2), mRNA”, gi|15042936|ref|NM_007365.1|[15042936]; 1316:
NM_007367, “Homo sapiens RNA binding protein (autoantigenic, hnRNP-associated with
lethal”, “yellow) (RALY), transcript variant 2, mRNA”,
gi|21396479|ref|NM_007367.2|[21396479]; 1317: NM_007373, “Homo sapiens soc-2
suppressor of clear homolog (C. elegans) (SHOC2), mRNA”,
gi|41281397|ref|NM_007373.2|[41281397]; 1318: NM_007374, “Homo sapiens sine oculis
homeobox homolog 6 (Drosophila) (SIX6), mRNA”, gi|6677978|ref|NM_007374.1|[6677978];
1319: NM_012083, “Homo sapiens frequently rearranged in advanced T-cell lymphomas 2
(FRAT2), mRNA”, gi|31317237|ref|NM_012083.2|[31317237]; 1320: NM_012086, “Homo
sapiens general transcription factor IIIC, polypeptide 3, 102 kDa (GTF3C3),”, mRNA,
gi|6912397|ref|NM_012086.1|[6912397]; 1321: NM_012087, “Homo sapiens general
transcription factor IIIC, polypeptide 5, 63 kDa (GTF3C5),”, mRNA,
gi|6912401|ref|NM_012087.1|[6912401]; 1322: NM_012096, “Homo sapiens adaptor protein
containing pH domain, PTB domain and leucine zipper”, “motif (APPL), mRNA”,
gi|6912241|ref|NM_012096.1|[6912241]; 1323: NM_012097, “Homo sapiens ADP-ribosylation
factor-like 5 (ARL5), transcript variant 1, mRNA”, gi|29542733|ref|NM_012097.2|[29542733];
1324: NM_012103, “Homo sapiens ancient ubiquitous protein 1 (AUP1), transcript variant 1,
mRNA”, gi|32313582|ref|NM_012103.2|[32313582]; 1325: NM_012104, “Homo sapiens beta-
site APP-cleaving enzyme (BACE), transcript variant a, mRNA”,
gi|21040369|ref|NM_012104.2|[21040369]; 1326: NM_012105, “Homo sapiens beta-site APP-
cleaving enzyme 2 (BACE2), transcript variant a, mRNA”,
gi|21040358|ref|NM_012105.3|[21040358]; 1327: NM_012111,“Homo sapiens AHA1,
activator of heat shock 90 kDa protein ATPase homolog 1”, “(yeast) (AHSA1), mRNA”,
gi|6912279|ref|NM_012111.1|[6912279]; 1328: NM_012112, “Homo sapiens TPX2,
microtubule-associated protein homolog (Xenopus laevis)”, “(TPX2), mRNA”,
gi|40354199|ref|NM_012112.4|[40354199]; 1329: NM_012124, “Homo sapiens cysteine and
histidine-rich domain (CHORD)-containing, zinc binding”, “protein 1 (CHORDC1), mRNA”,
gi|6912303|ref|NM_012124.1|[6912303]; 1330: NM_012130, “Homo sapiens claudin 14
(CLDN 14), transcript variant 2, mRNA”, gi|21536295|ref|NM_012130.2|[21536295]; 1331:
NM_012133, “Homo sapiens coatomer protein complex, subunit gamma 2 (COPG2), mRNA”,
gi|6912319|ref|NM_012133.1|[6912319]; 1332: NM_012139, Homo sapiens deafness locus
associated putative guanine nucleotide exchange, “factor (DELGEF), mRNA”,
gi|40548400|ref|NM_012139.2|[40548400]; 1333: NM_012144, “Homo sapiens dynein,
axonemal, intermediate polypeptide 1 (DNAI1), mRNA”,
gi|22212919|ref|NM_012144.2|[22212919]; 1334: NM_012152, “Homo sapiens endothelial
differentiation, lysophosphatidic acid”, “G-protein-coupled receptor, 7 (EDG7), mRNA”,
gi|6912347|ref|NM_012152.1|[6912347]; 1335: NM_012160, “Homo sapiens F-box and
leucine-rich repeat protein 4 (FBXL4), mRNA”, gi|21536437|ref|NM_012160.3|[21536437];
1336: NM_012164, “Homo sapiens F-box and WD-40 domain protein 2 (FBXW2), mRNA”,
gi|7549806|ref|NM_012164.2|[7549806]; 1337: NM_012168, “Homo sapiens F-box only protein
2 (FBXO2), mRNA”, gi|15812197|ref|NM_012168.2|[15812197]; 1338: NM_012170, “Homo
sapiens F-box only protein 22 (FBXO22), transcript variant 2, mRNA”,
gi|22547147|ref|NM_012170.2|[22547147]; 1339: NM_012177, “Homo sapiens F-box only
protein 5 (FBXO5), mRNA”, gi|15812190|ref|NM_012177.2|[15812190]; 1340: NM_012179,
“Homo sapiens F-box only protein 7 (FBXO7), mRNA”,
gi|15812192|ref|NM_012179|[15812192]; 1341: NM_012182, “Homo sapiens forkhead box
B1 (FOXB1), mRNA”, gi|11386194|ref|NM_012182.1|[11386194]; 1342: NM_012183, “Homo
sapiens forkhead box D3 (FOXD3), mRNA”, gi|6912371|ref|NM_012183.1|[6912371]; 1343:
NM_012191, “Homo sapiens putative tumor suppressor (FUS2), mRNA”,
gi|6912379|ref|NM_012191.1|[6912379]; 1344: NM_012192, “Homo sapiens fracture callus 1
homolog (rat) (FXC1), mRNA”, gi|29837656|ref|NM_012192.2|[29837656]; 1345: NM_012198,
“Homo sapiens grancalcin, EF-hand calcium binding protein (GCA), mRNA”,
gi|21614521|ref|NM_012198.2|[21614521]; 1346: NM_012204, “Homo sapiens general
transcription factor IIIC, polypeptide 4, 90 kDa (GTF3C4),”, mRNA,
gi|6912399|ref|NM_012204.1|[6912399]; 1347: NM_012222, “Homo sapiens mutY homolog
(E. coli) (MUTYH), mRNA”, gi|6912519|ref|NM_012222.1|[6912519]; 1348: NM_012237,
Homo sapiens sirtuin (silent mating type information regulation 2 homolog) 2 (S., “cerevisiae)
(SIRT2), transcript variant 1, mRNA”, gi|13775599|ref|NM_012237.2|[13775599]; 1349:
NM_012242, “Homo sapiens dickkopf homolog 1 (Xenopus laevis) (DKK1), mRNA”,
gi|7110718|ref|NM_012242.1|[7110718]; 1350: NM_012254, “Homo sapiens solute carrier
family 27 (fatty acid transporter), member 5”, “(SLC27A5), mRNA”,
gi|13325056|ref|NM_012254.1|[13325056]; 1351: NM_012256, “Homo sapiens zinc finger
protein 212 (ZNF212), mRNA”, gi|24797064|ref|NM_012256.2|[24797064]; 1352: NM_012259,
“Homo sapiens hairy/enhancer-of-split related with YRPW motif 2 (HEY2), mRNA”,
gi|6912413|ref|NM_012259.1|[6912413]; 1353: NM_012265, “Homo sapiens chromosome 22
open reading frame 3 (C22orf3), mRNA”, gi|11072100|ref|NM_012265.1|[11072100]; 1354:
NM_012281, “Homo sapiens potassium voltage-gated channel, Shal-related subfamily, member
2”, “(KCND2), mRNA”, gi|27436982|ref|NM_012281.2|[27436982]; 1355: NM_012285,
“Homo sapiens potassium voltage-gated channel, subfamily H (eag-related), member”, “4
(KCNH4), mRNA”, gi|6912445|ref|NM_012285.1|[6912445]; 1356: NM_012289, “Homo
sapiens kelch-like ECH-associated protein 1 (KEAP1), mRNA”,
gi|22027641|ref|NM_012289.2|[22027641]; 1357: NM_012311, “Homo sapiens KIN, antigenic
determinant of recA protein homolog (mouse) (KIN),”, mRNA,
gi|40068516|ref|NM_012311.2|[40068516]; 1358: NM_012327, “Homo sapiens
phosphatidylinositol glycan, class N (PIGN), transcript variant 2,”, mRNA,
gi|34328903|ref|NM_012327.3|[34328903]; 1359: NM_012339, “Homo sapiens transmembrane
4 superfamily member tetraspan NET-7 (NET-7), mRNA”,
gi|21264576|ref|NM_012339.2|[21264576]; 1360: NM_012342, Homo sapiens BMP and activin
membrane-bound inhibitor homolog (Xenopus laevis), “(BAMBI), mRNA”,
gi|6912533|ref|NM_012342.1|[6912533]; 1361: NM_012381, “Homo sapiens origin recognition
complex, subunit 3-like (yeast) (ORC3L),”, “transcript variant 2, mRNA”,
gi|32483366|ref|NM_012381.2|[32483366]; 1362: NM_012392, Homo sapiens PEF protein with
a long N-terminal hydrophobic domain (peflin), “(PEF), mRNA”,
gi|6912581|ref|NM_012392.1|[6912581]; 1363: NM_012396, “Homo sapiens pleckstrin
homology-like domain, family A, member 3 (PHLDA3), mRNA”,
gi|6912589|ref|NM_012396.1|[6912589]; 1364: NM_012399, “Homo sapiens
phosphotidylinositol transfer protein, beta (PLTPNB), mRNA”,
gi|19923401|ref|NM_012399.2|[19923401]; 1365: NM_012402, Homo sapiens ADP-
ribosylation factor interacting protein 2 (arfaptin 2), “(ARFIP2), mRNA”,
gi|38569401|ref|NM_012402.2|[38569401]; 1366: NM_012407, “Homo sapiens protein kinase
C, alpha binding protein (PRKCABP), mRNA”, gi|7110696|ref|NM_012407.1|[7110696]; 1367:
NM_012424 “Homo sapiens ribosomal protein S6 kinase, 52 kDa, polypeptide 1 (RPS6KC1),
mRNA”, gi|19923722|ref|NM_012424.2|[19923722]; 1368: NM_012425, “Homo sapiens Ras
suppressor protein 1 (RSU1), transcript variant 1, mRNA”,
gi|34577084|ref|NM_012425.3|[34577084]; 1369: NM_012427, “Homo sapiens kallikrein 5
(KLK5), mRNA”, gi|22208993|ref|NM_012427.3|[22208993]; 1370: NM_012430, “Homo
sapiens SEC22 vesicle trafficking protein-like 2 (S. cerevisiae) (SEC22L2),”, mRNA,
gi|14591918|ref|NM_012430.2|[14591918]; 1371: NM_012445, “Homo sapiens spondin 2,
extracellular matrix protein (SPON2), mRNA”, gi|6912681|ref|NM_012445.1|[6912681]; 1372:
NM_012448, “Homo sapiens signal transducer and activator of transcription 5B (STAT5B),
mRNA”, gi|42519913|ref|NM_012448.3|[42519913]; 1373: NM_012450, “Homo sapiens solute
carrier family 13 (sodium/sulfate symporters), member 4”, “(SLC13A4), mRNA”,
gi|31795545|ref|NM_012450.2|[31795545]; 1374: NM_012451, “Homo sapiens synaptogyrin 4
(SYNGR4), mRNA”, gi|22035701|ref|NM_012451.2|[22035701]; 1375: NM_012456, Homo
sapiens translocase of inner mitochondrial membrane 10 homolog (yeast), “(TIMM10), mRNA”,
gi|6912707|ref|NM_012456.1|[6912707]; 1376: NM_012458, Homo sapiens translocase of inner
mitochondrial membrane 13 homolog (yeast), “(TIMM13), nuclear gene encoding mitochondrial
protein, mRNA”, gi|27436898|ref|NM_012458.2|[27436898]; 1377: NM_012459, Homo sapiens
translocase of inner mitochondrial membrane 8 homolog B (yeast), “(TIMM8B), mRNA”,
gi|6912711|ref|NM_012459.1|[6912711]; 1378: NM_012460, Homo sapiens translocase of inner
mitochondrial membrane 9 homolog (yeast), “(TIMM9), mRNA”,
gi|21359892|ref|NM_012460.2|[21359892]; 1379: NM_012461, “Homo sapiens TERF1
(TRF1)-interacting nuclear factor 2 (TINF2), mRNA”, gi|6912715|ref|NM_012461.1|[6912715];
1380: NM_012481, “Homo sapiens zinc finger protein, subfamily 1A, 3 (Aiolos) (ZNFN1A3),
transcript”, “variant 1, mRNA”, gi|38045957|ref|NM_012481.3|[38045957]; 1381: NM_012482,
“Homo sapiens zinc finger protein 281 (ZNF281), mRNA”,
gi|40255235|ref|NM_012482.3|[40255235]; 1382: NM_013232, “Homo sapiens programmed
cell death 6 (PDCD6), mRNA”, gi|22027539|ref|NM_013232.2|[22027539]; 1383: NM_013235,
“Homo sapiens nuclear RNase III Drosha (RNASE3L), mRNA”,
gi|21359821|ref|NM_013235.2|[21359821]; 1384: NM_013238, “Homo sapiens DnaJ (Hsp40)
homolog, subfamily D, member 1 (DNAJD1), mRNA”,
gi|7019452|ref|NM_013238.1|[7019452]; 1385: NM_013241, “Homo sapiens formin homology
2 domain containing 1 (FHOD1), mRNA”, gi|7019374|ref|NM_013241.1|[7019374]; 1386:
NM_013242, “Homo sapiens likely ortholog of mouse gene trap locus 3 (GTL3), mRNA”,
gi|42716281|ref|NM_013242.2|[42716281]; 1387: NM_013248, “Homo sapiens NTF2-like
export factor 1 (NXT1), mRNA”, gi|20127526|ref|NM_013248.2|[20127526]; 1388:
NM_013250, “Homo sapiens zinc finger protein 215 (ZNF215), mRNA”,
gi|7019582|ref|NM_013250.1|[7019582]; 1389: NM_013254, “Homo sapiens TANK-binding
kinase 1 (TBK1), mRNA”, gi|19743810|ref|NM_013254.2|[19743810]; 1390: NM_013256,
“Homo sapiens zinc finger protein 180 (HHZ168) (ZNF180), mRNA”,
gi|7019578|ref|NM_013256.1|[7019578]; 1391: NM_013260, “Homo sapiens transcriptional
regulator protein (HCNGP), mRNA”, gi|21361710|ref|NM_013260.3|[21361710]; 1392:
NM_013263, “Homo sapiens bromodomain containing 7 (BRD7), mRNA”,
gi|41350211|ref|NM_013263.2|[41350211]; 1393: NM_013264, “Homo sapiens DEAD (Asp-
Glu-Ala-Asp) box polypeptide 25 (DDX25), mRNA”,
gi|21327696|ref|NM_013264.2|[21327696]; 1394: NM_013266, “Homo sapiens catenin
(cadherin-associated protein), alpha 3 (CTNNA3), mRNA”,
gi|7019570|ref|NM_013266.1|[7019570]; 1395: NM_013267, “Homo sapiens liver
mitochondrial glutaminase (GA), nuclear gene encoding”, “mitochondrial protein, transcript
variant 1, mRNA”, gi|20336213|ref|NM_013267.2|[20336213]; 1396: NM_013270, “Homo
sapiens testes-specific protease 50 (TSP50), mRNA”,
gi|31543829|ref|NM_013270.2|[31543829]; 1397: NM_013274, “Homo sapiens polymerase
(DNA directed), lambda (POLL), mRNA”, gi|38146101|ref|NM_013274.2|[38146101]; 1398:
NM_013275, “Homo sapiens ankyrin repeat domain 11 (ANKRD11), mRNA”,
gi|40786546|ref|NM_013275.3|[40786546]; 1399: NM_013283, “Homo sapiens methionine
adenosyltransferase II, beta (MAT2B), transcript variant”, “1, mRNA”,
gi|33519456|ref|NM_013283.3|[33519456]; 1400: NM_013284, “Homo sapiens polymerase
(DNA directed), mu (POLM), mRNA”, gi|7019492|ref|NM_013284.1|[7019492]; 1401:
NM_013285, “Homo sapiens nucleolar GTPase (HUMAUANTIG), mRNA”,
gi|7019418|ref|NM_013285.1|[7019418]; 1402: NM_013286, “Homo sapiens chromosome
3p21.1 gene sequence (HUMAGCGB), mRNA”, gi|31712021|ref|NM_013286.2|[31712021];
1403: NM_013301, “Homo sapiens protein predicted by clone 23882 (HSU79303), mRNA”,
gi|9558742|ref|NM_013301.1|[9558742]; 1404: NM_013312, “Homo sapiens hook homolog 2
(Drosophila) (HOOK2), mRNA”, gi|7019410|ref|NM_013312.1|[7019410]; 1405: NM_013322,
“Homo sapiens sorting nexin 10 (SNX10), mRNA”, gi|23111022|ref|NM_013322.2|[23111022];
1406: NM_013324, “Homo sapiens cytokine inducible SH2-containing protein (CISH),
transcript”, “variant 1, mRNA”, gi|21614504|ref|NM_013324.4|[21614504]; 1407: NM_013326,
“Homo sapiens chromosome 18 open reading frame 8 (C18orf8), mRNA”,
gi|21361441|ref|NM_013326.2|[21361441]; 1408: NM_013330, “Homo sapiens non-metastatic
cells 7, protein expressed in”, “(nucleoside-diphosphate kinase) (NME7), transcript variant 1,
mRNA”, gi|37574616|ref|NM_013330.3|[37574616]; 1409: NM_013333, “Homo sapiens epsin
1 (EPN1), mRNA”, gi|41350200|ref|NM_013333.2|[41350200]; 1410: NM_013335, “Homo
sapiens GDP-mannose pyrophosphorylase A (GMPPA), mRNA”,
gi|31881778|ref|NM_013335.2|[31881778]; 1411: NM_013336, “Homo sapiens Sec61 alpha 1
subunit (S. cerevisiae) (SEC61A1), mRNA”, gi|14591931|ref|NM_013336.2|[14591931]; 1412:
NM_013338, “Homo sapiens asparagine-linked glycosylation 5 homolog (yeast,”, “dolichyl-
phosphate beta-glucosyltransferase) (ALG5), mRNA”,
gi|38176301|ref|NM_013338.3|[38176301]; 1413: NM_013339, “Homo sapiens asparagine-
linked glycosylation 6 homolog (yeast,”, “alpha-1,3-glucosyltransferase) (ALG6), mRNA”,
gi|38026891|ref|NM_013339.2|[38026891]; 1414: NM_013341, “Homo sapiens hypothetical
protein PTD004 (PTD004), mRNA”, gi|24431968|ref|NM_013341.2|[24431968]; 1415:
NM_013342, “Homo sapiens TCF3 (E2A) fusion partner (in childhood Leukemia) (TFPT),
mRNA”, gi|7019370|ref|NM_013342.1|[7019370]; 1416: NM_013343, “Homo sapiens loss of
heterozygosity, 3, chromosomal region 2, gene A (LOH3CR2A),”, mRNA,
gi|7106370|ref|NM_013343.1|[7106370]; 1417: NM_013345, “Homo sapiens G protein-coupled
receptor 132 (GPR132), mRNA”, gi|30181231|ref|NM_013345.2|[30181231]; 1418:
NM_013348, “Homo sapiens potassium inwardly-rectifying channel, subfamily J, member 14”,
“(KCNJ14), transcript variant 1, mRNA”, gi|25777633|ref|NM_013348.2|[25777633]; 1419:
NM_013366, “Homo sapiens anaphase promoting complex subunit 2 (ANAPC2), mRNA”,
gi|41327747|ref|NM_013366.3|[41327747]; 1420: NM_013374, “Homo sapiens programmed
cell death 6 interacting protein (PDCD6IP), mRNA”, gi|22027537|ref|NM_013374.2|[22027537];
1421: NM_013375, “Homo sapiens activator of basal transcription 1 (ABT1), mRNA”,
gi|17572813|ref|NM_013375.2|[17572813]; 1422: NM_013380, “Homo sapiens zinc finger
protein 228 (ZNF228), mRNA”, gi|34932234|ref|NM_013380.2|[34932234]; 1423: NM_013381,
“Homo sapiens thyrotropin-releasing hormone degrading ectoenzyme (TRHDE), mRNA”,
gi|7019560|ref|NM_013381.1|[7019560]; 1424: NM_013382, “Homo sapiens protein-O-
mannosyltransferase 2 (POMT2), mRNA”, gi|32455270|ref|NM_013382.3|[32455270]; 1425:
NM_013384, “Homo sapiens LAG1 longevity assurance homolog 2 (S. cerevisiae) (LASS2),”,
“transcript variant 3, mRNA”, gi|32455253|ref|NM_013384.3|[32455253]; 1426: NM_013386,
“Homo sapiens calcium-binding transporter (DKFZp586G0123), mRNA”,
gi|33598953|ref|NM_013386.2|[33598953]; 1427: NM_013387, “Homo sapiens ubiquinol-
cytochrome c reductase complex (7.2 kD) (HSPC051), mRNA”,
gi|41281884|ref|NM_013387.2|[41281884]; 1428: NM_013392, “Homo sapiens nuclear receptor
binding protein (NRBP), mRNA”, gi|7019332|ref|NM_013392.1|[7019332]; 1429: NM_013400,
“Homo sapiens replication initiator 1 (REPIN1), mRNA”,
gi|7019516|ref|NM_013400.1|[7019516]; 1430: NM_013403, “Homo sapiens striatin,
calmodulin binding protein 4 (STRN4), mRNA”, gi|7019572|ref|NM_013403.1|[7019572]; 1431:
NM_013441, “Homo sapiens Down syndrome critical region gene 1-like 2 (DSCR1L2),
mRNA”, gi|38455419|ref|NM_013441.2|[338455419]; 1432: NM_013442, “Homo sapiens
stomatin (EPB72)-like 2 (STOML2), mRNA”, gi|7305502|ref|NM_013442.1|[7305502]; 1433:
NM_014012, “Homo sapiens RAS (RAD and GEM)-like GTP-binding (REM), mRNA”,
gi|35493898|ref|NM_014012.4|[35493898]; 1434: NM_014020, “Homo sapiens LR8 protein
(LR8), mRNA”, gi|21361500|ref|NM_014020.2|[21361500]; 1435: NM_014035, “Homo sapiens
sorting nexing 24 (SNX24), mRNA”, gi|7662654|ref|NM_014035.1|[7662654]; 1436:
NM_014038, “Homo sapiens basic leucine zipper and W2 domains 2 (BZW2), mRNA”,
gi|7661743|ref|NM_014038.1|[7661743]; 1437: NM_014041, “Homo sapiens signal peptidase
12 kDa (SPC12), mRNA”, gi|7661745|ref|NM_014041.1|[7661745]; 1438: NM_014044, “Homo
sapiens unc-50 homolog (C. elegans) (UNC50), mRNA”,
gi|37059764|ref|NM_014044.4|[37059764]; 1439: NM_014045, “Homo sapiens low density
lipoprotein receptor-related protein 10 (LRP10), mRNA”,
gi|32490558|ref|NM_014045.21|[32490558]; 1440: NM_014047, “Homo sapiens HSPC023
protein (HSPC023), mRNA”, gi|7661741|ref|NM_014047.1|[7661741]; 1441: NM_014048,
“Homo sapiens myocardin-related transcription factor B (MRTF-B), mRNA”,
gi|38569479|ref|NM_014048.3|[38569479]; 1442: NM_014051, “Homo sapiens transmembrane
protein 14A (TMEM14A), mRNA”, gi|32261328|ref|NM_014051.2|[32261328]; 1443:
NM_014055, Homo sapiens carnitine deficiency-associated gene expressed in ventricle 1,
“(CDV-1), mRNA”, gi|32526900|ref|NM_014055.2|[32526900]; 1444: NM_014059, “Homo
sapiens response gene to complement 32 (RGC32), mRNA”,
gi|7662650|ref|NM_014059.1|[7662650]; 1445: NM_014060, “Homo sapiens malignant T cell
amplified sequence 1 (MCTS1), mRNA”, gi|7662501|ref|NM_014060.1|[7662501]; 1446:
NM_014065, “Homo sapiens HT001 protein (HT001), mRNA”,
gi|33469986|ref|NM_014065.2|[33469986]; 1447: NM_014070, “Homo sapiens chromosome 6
open reading frame 15 (C6orf15), mRNA”, gi|7662666|ref|NM_014070.1|[7662666]; 1448:
NM_014099,, ref|NM_014099.1|[7662610], This record was temporarily removed by RefSeq
staff for additional review.,, 1449: NM_014109, “Homo sapiens PRO2000 protein (PRO2000),
mRNA”, gi|24497617|ref|NM_014109.2|[24497617]; 1450: NM_014113, “Homo sapiens
PRO0038 protein (PRO0038), mRNA”, gi|7662519|ref|NM_014113.1|[7662519]; 1451:
NM_014117, “Homo sapiens PRO0149 protein (PRO0149), mRNA”,
gi|38016918|ref|NM_014117.2|[38016918]; 1452: NM_014124,, ref|NM_014124.1|[7662541],
This record was temporarily removed by RefSeq staff for additional review.,, 1453:
NM_014133,, ref|NM_014133.1|[7662573], This record was temporarily removed by RefSeq
staff for additional review.,, 1454: NM_014135,, ref|NM_014135.1|[7662577], This record was
temporarily removed by RefSeq staff for additional review.,, 1455: NM_014140, “Homo
sapiens SWI/SNF related, matrix associated, actin dependent regulator of”, “chromatin,
subfamily a-like 1 (SMARCAL1), mRNA”, gi|21071059|ref|NM_014140.2|[21071059]; 1456:
NM_014144, “Homo sapiens chromosome 11 open reading frame 21 (C11orf21), mRNA”,
gi|7662662|ref|NM_014144.1|[7662662]; 1457: NM_014145, “Homo sapiens chromosome 20
open reading frame 30 (C20orf30), mRNA”, gi|42476067|ref|NM_014145.3|[42476067]; 1458:
NM_014155, “Homo sapiens HSPC063 protein (HSPC063), mRNA”,
gi|7661765|ref|NM_014155.1|[7661765]; 1459: NM_014161, “Homo sapiens mitochondrial
ribosomal protein L18 (MRPL18), nuclear gene encoding”, “mitochondrial protein, mRNA”,
gi|21265079|ref|NM_014161.2|[21265079]; 1460: NM_014162, “Homo sapiens HSPC072
protein (HSPC072), mRNA”, gi|7661779|ref|NM_014162.1|[7661779]; 1461: NM_014164,
“Homo sapiens FXYD domain containing ion transport regulator 5 (FXYD5), mRNA”,
gi|21618360|ref|NM_014164.3|[21618360]; 1462: NM_014165, “Homo sapiens chromosome 6
open reading frame 66 (C6orf66), mRNA”, gi|7661785|ref|NM_014165.1|[7661785]; 1463:
NM_014166, “Homo sapiens vitamin D receptor interacting protein (VDRIP), mRNA”,
gi|40254874|ref|NM_014166.2|[40254874]; 1464: NM_014171, “Homo sapiens postsynaptic
protein CRIPT (CRIPT), mRNA”, gi|41350204|ref|NM_014171.3|[41350204]; 1465:
NM_014173, “Homo sapiens HSPC142 protein (HSPC142), mRNA”,
gi|7661801|ref|NM_014173.1|[7661801]; 1466: NM_014174, “Homo sapiens thymocyte protein
thy28 (THY28), transcript variant 1, mRNA”, gi|40806217|ref|NM_014174.2|[40806217]; 1467:
NM_014179, “Homo sapiens HSPC157 protein (HSPC157), mRNA”,
gi|7661813|ref|NM_014179.1|[7661813]; 1468: NM_014185,, ref|NM_0414185.1|[7661825],
This record was replaced or removed. See revision history for details.,, 1469: NM_014187,
“Homo sapiens HSPC171 protein (HSPC171), mRNA”,
gi|7661829|ref|NM_014187.1|[7661829]; 1470: NM_014191, “Homo sapiens sodium channel,
voltage gated, type VIII, alpha (SCN8A), mRNA”, gi|7657543|ref|NM_014191.1|[7657543];
1471: NM_014205, “Homo sapiens chromosome 11 open reading frame 5 (C11orf5), mRNA”,
gi|42716303|ref|NM_014205.2|[42716303]; 1472: NM_014206, “Homo sapiens chromosome 11
open reading frame 10 (C11orf10), mRNA”, gi|7656933|ref|NM_014206.1|[7656933]; 1473:
NM_014211, “Homo sapiens gamma-aminobutyric acid (GABA) A receptor, pi (GABRP),
mRNA”, gi|7657105|ref|NM_014211.1|[7657105]; 1474: NM_014225, “Homo sapiens protein
phosphatase 2 (formerly 2A), regulatory subunit A (PR 65),”, “alpha isoform (PPP2R1A),
mRNA”, gi|32455242|ref|NM_014225.3|[32455242]; 1475: NM_014226, “Homo sapiens renal
tumor antigen (RAGE), mRNA”, gi|7657497|ref|NM_014226.1|[7657497]; 1476: NM_014234,
“Homo sapiens hydroxysteroid (17-beta) dehydrogenase 8 (HSD17B8), mRNA”,
gi|20143980|ref|NM_014234.3|[20143980]; 1477: NM_014235, “Homo sapiens ubiquitin-like 4
(UBL4), mRNA”, gi|40254852|ref|NM_014235.2|[40254852]; 1478: NM_014236, “Homo
sapiens glyceronephosphate O-acyltransferase (GNPAT), mRNA”,
gi|7657133|ref|NM_014236.1|[7657133]; 1479: NM_014239, “Homo sapiens eukaryotic
translation initiation factor 2B, subunit 2 beta, 39 kDa”, “(EIF2B2), mRNA”,
gi|7657057|ref|NM_014239.1|[7657057]; 1480: NM_014243, Homo sapiens a disintegrin-like
and metalloprotease (reprolysin type) with, “thrombospondin type 1 motif, 3 (ADAMTS3),
mRNA”, gi|21265036|ref|NM_014243.1|[21265036]; 1481: NM_014245, “Homo sapiens ring
finger protein 7 (RNF7), transcript variant 1, mRNA”,
gi|34304329|ref|NM_014245.2|[34304329]; 1482: NM_014248, “Homo sapiens ring-box 1
(RBX1), mRNA”, gi|22091459|ref|NM_014248.2|[22091459]; 1483: NM_014252, Homo
sapiens solute carrier family 25 (mitochondrial carrier; ornithine, “transporter) member 15
(SLC25A15), nuclear gene encoding mitochondrial protein,”, mRNA,
gi|7657584|ref|NM_014252.1|[7657584]; 1484: NM_014258, “Homo sapiens synaptonemal
complex protein 2 (SYCP2), mRNA”, gi|38373672|ref|NM_014258.2|[38373672]; 1485:
NM_014262, “Homo sapiens leprecan-like 2 protein (LEPREL2), mRNA”,
gi|28466982|ref|NM_014262.2|[28466982]; 1486: NM_014273, Homo sapiens a disintegrin-like
and metalloprotease (reprolysin type) with, “thrombospondin type 1 motif, 6 (ADAMTS6),
mRNA”, gi|21536389|ref|NM_014273.2|[21536389]; 1487: NM_014275, “Homo sapiens
mannosyl (alpha-1,3-)-glycoprotein”, “beta-1,4-N-acetylglucosaminyltransferase, isoenzyme B
(MGAT4B), transcript”, “variant 1, mRNA”, gi|16915933|ref|NM_014275.2|[16915933]; 1488:
NM_014276, Homo sapiens recombining binding protein suppressor of hairless, “(Drosophila)-
like (RBPSUHL), mRNA”, gi|34577080|ref|NM_014276.2|[34577080]; 1489: NM_014278,
“Homo sapiens heat shock protein (hsp110 family) (APG-1), mRNA”,
gi|31541940|ref|NM_014278.2|[31541940]; 1490: NM_014283, “Homo sapiens chromosome 1
open reading frame 9 (C1orf9), mRNA”, gi|29837653|ref|NM_014283.2|[29837653]; 1491:
NM_014288, “Homo sapiens integrin beta 3 binding protein (beta3-endonexin) (ITGB3BP),
mRNA”, gi|27597074|ref|NM_014288.3|[27597074]; 1492: NM_014290, “Homo sapiens tudor
repeat associator with PCTAIRE 2 (PCTAIRE2BP), mRNA”,
gi|24307950|ref|NM_014290.1|[24307950]; 1493: NM_014296, “Homo sapiens calpain 7
(CAPN7), mRNA”, gi|41327720|ref|NM_014296.2|[41327720]; 1494: NM_014301, “Homo
sapiens nitrogen fixation cluster-like (NIFU), mRNA”,
gi|24307952|ref|NM_041301.1|[24307952]; 1495: NM_014302, “Homo sapiens Sec61 gamma
subunit (SEC61G), mRNA”, gi|14591933|ref|NM_014302.2|[14591933]; 1496: NM_014303,
“Homo sapiens pescadillo homolog 1, containing BRCT domain (zebrafish) (PES1),”, mRNA,
gi|22091458|ref|NM_014303.2|[22091458]; 1497: NM_014305, “Homo sapiens TDP-glucose
4,6-dehydratase (TGDS), mRNA”, gi|7657640|ref|NM_014305.1|[7657640]; 1498: NM_014308,
“Homo sapiens phosphoinositide-3-kinase, regulatory subunit, polypeptide p101”, “(P101-
PI3K), mRNA”, gi|7657432|ref|NM_014308.1|[7657432]; 1499: NM_014315, “Homo sapiens
kelch domain containing 2 (KLHDC2), mRNA”, gi|7657300|ref|NM_014315.1|[7657300]; 1500:
NM_014317, “Homo sapiens trans-prenyltransferase (TPRT), mRNA”,
gi|11863164|ref|NM_014317.2|[11863164]; 1501: NM_014319, “Homo sapiens integral inner
nuclear membrane protein (MAN1), mRNA”, gi|36287116|ref|NM_014319.3|[36287116]; 1502:
NM_014322, “Homo sapiens opsin 3 (encephalopsin, panopsin) (OPN3), mRNA”,
gi|7657070|ref|NM_014322.1|[7657070]; 1503: NM_014329, “Homo sapiens autoantigen
(RCD-8), mRNA”, gi|21361430|ref|NM_014329.2|[21361430]; 1504: NM_014338, “Homo
sapiens phosphatidylserine decarboxylase (PISD), mRNA”,
gi|34147578|ref|NM_014338.3|[34147578]; 1505: NM_014342, “Homo sapiens mitochondrial
carrier homolog 2 (C. elegans) (MTCH2), nuclear gene”, “encoding mitochondrial protein,
mRNA”, gi|40254847|ref|NM_014342.2|[40254847]; 1506: NM_014344, “Homo sapiens four
jointed box 1 (Drosophila) (FJX1), mRNA”, gi|18765710|ref|NM_014344.2|[18765710]; 1507:
NM_014348, “Homo sapiens POM121 membrane glycoprotein-like 1 (rat) (POM121L1),
mRNA”, gi|7657468|ref|NM_014348.1|[7657468]; 1508: NM_014360, “Homo sapiens NK2
transcription factor related, locus 8 (Drosophila) (NKX2-8),”, mRNA,
gi|31377776|ref|NM_014360.2|[31377776]; 1509: NM_014361, “Homo sapiens contactin 5
(CNTN5), transcript variant 1, mRNA”, gi|28373127|ref|NM_014361.2|[28373127]; 1510:
NM_014364 “Homo sapiens glyceraldehyde-3-phosphate dehydrogenase, spermatogenic
(GAPDS),”, mRNA, gi|34222311|ref|NM_014364.3|[34222311]; 1511: NM_014365 “Homo
sapiens heat shock 27 kDa protein 8 (HSPB8), mRNA”,
gi|38016940|ref|NM_014365.2|[38016940]; 1512: NM_014366, “Homo sapiens nucleostemin
(NS), mRNA”, gi|37497106|ref|NM_014366.3|[37497106]; 1513: NM_014368, “Homo sapiens
LIM homeobox 6 (LHX6), transcript variant 1, mRNA”,
gi|40549416|ref|NM_014368.2|[40549416]; 1514: NM_014372, “Homo sapiens ring finger
protein 11 (RNF11), mRNA”, gi|34452682|ref|NM_014372.3|[34452682]; 1515: NM_014384,
“Homo sapiens acyl-Coenzyme A dehydrogenase family, member 8 (ACAD8), mRNA”,
gi|7656848|ref|NM_014384.1|[7656848]; 1516: NM_014390, “Homo sapiens staphylococcal
nuclease domain containing 1 (SND1), mRNA”, gi|7657430|ref|NM_014390.1|[7657430]; 1517:
NM_014391, “Homo sapiens ankyrin repeat domain 1 (cardiac muscle) (ANKRD1), mRNA”,
gi|38327521|ref|NM_014391.2|[38327521]; 1518: NM_014402, “Homo sapiens low molecular
mass ubiquinone-binding protein (9.5 kD) (QP-C),”, “nuclear gene encoding mitochondrial
protein, mRNA”, gi|27894387|ref|NM_014402.2|[27894387]; 1519: NM_014409, “Homo
sapiens TAF5-like RNA polymerase II, p300/CBP-associated factor”, “(PCAF)-associated
factor, 65 kDa (TAF5L), mRNA”, gi|21269865|ref|NM_014409.2|[21269865]; 1520:
NM_014415, “Homo sapiens zinc finger protein (ZNF-U69274), mRNA”,
gi|7657702|ref|NM_014415.1|[7657702]; 1521: NM_014421, “Homo sapiens dickkopf homolog
2 (Xenopus laevis) (DKK2), mRNA”, gi|7657022|ref|NM_014421.1|[7657022]; 1522:
NM_014426, “Homo sapiens sorting nexin 5 (SNX5), transcript variant 2, mRNA”,
gi|23111045|ref|NM_014426.2|[23111045]; 1523: NM_014427, “Homo sapiens copine VII
(CPNE7), transcript variant 2, mRNA”, gi|25141326|ref|NM_014427.3|[25141326]; 1524:
NM_014429, “Homo sapiens microrchidia homolog (mouse) (MORC), mRNA”,
gi|7657340|ref|NM_014429.1|[7657340]; 1525: NM_014430, “Homo sapiens cell death-
inducing DFFA-like effector b (CIDEB), mRNA”, gi|7656978|ref|NM_014430.1|[7656978];
1526: NM_014432, “Homo sapiens interleukin 20 receptor, alpha (IL20RA), mRNA”,
gi|31083155|ref|NM_014432.2|[31083155]; 1527: NM_014437, “Homo sapiens solute carrier
family 39 (zinc transporter), member 1 (SLC39A1),”, mRNA,
gi|34147669|ref|NM_014437.3|[34147669]; 1528: NM_014440, “Homo sapiens interleukin 1
family, member 6 (epsilon) (IL1F6), mRNA”, gi|7657091|ref|NM_014440.1|[7657091]; 1529:
NM_014453, “Homo sapiens putative breast adenocarcinoma marker (32 kD) (BC-2),
transcript”, “variant 1, mRNA”, gi|38372936|ref|NM_014453.2|[38372936]; 1530: NM_014459,
“Homo sapiens protocadherin 17 (PCDH17), mRNA”,
gi|14589926|ref|NM_014459.2|[14589926]; 1531: NM_014462, “Homo sapiens LSM1
homolog, U6 small nuclear RNA associated (S. cerevisiae)”, “(LSM1), mRNA”,
gi|7657312|ref|NM_014462.1|[7657312]; 1532: NM_014466, “Homo sapiens tektin 2
(testicular) (TEKT2), mRNA”, gi|16507949|ref|NM_014466.2|[16507949]; 1533: NM_014471,
“Homo sapiens serine protease inhibitor, Kazal type 4 (SPINK4), mRNA”,
gi|7657452|ref|NM_014471.1|[7657452]; 1534: NM_014484, “Homo sapiens molybdenum
cofactor synthesis 3 (MOCS3), mRNA”, gi|31652257|ref|NM_014484.3|[31652257]; 1535:
NM_014504, “Homo sapiens RAB guanine nucleotide exchange factor (GEF) 1 (RABGEF1),
mRNA”, gi|7657495|ref|NM_014504.1|[7657495]; 1536: NM_014505, “Homo sapiens
potassium large conductance calcium-activated channel, subfamily M,”, “beta member 4
(KCNMB4), mRNA”, gi|26051274|ref|NM_014505.4|[26051274]; 1537: NM_014506, “Homo
sapiens torsin family 1, member B (torsin B) (TOR1B), mRNA”,
gi|14149652|ref|NM_014506.1|[14149652]; 1538: NM_014507, Homo sapiens malonyl-
CoA: acyl carrier protein transacylase (malonyltransferase), “(MT), mRNA”,
gi|27477044|ref|NM_014507.1|[27477044]; 1539: NM_014517, “Homo sapiens upstream
binding protein 1 (LBP-1a) (UBP1), mRNA”, gi|31543907|ref|NM_014517.2|[31543907]; 1540:
NM_014520, “Homo sapiens MYB binding protein (P160) 1a (MYBBP1A), mRNA”,
gi|7657350|ref|NM_014520.1|[7657350]; 1541: NM_014548, “Homo sapiens tropomodulin 2
(neuronal) (TMOD2), mRNA”, gi|40789262|ref|NM_014548.2|[40789262]; 1542: NM_014563,
“Homo sapiens spondyloepiphyseal dysplasia, late (SEDL), mRNA”,
gi|38044279|ref|NM_014563.2|[38044279]; 1543: NM_014565, “Homo sapiens olfactory
receptor, family 1, subfamily A, member 1 (OR1A1), mRNA”,
gi|7657420|ref|NM_014565.1|[7657420]; 1544: NM_014571, “Homo sapiens hairy/enhancer-of-
split related with YRPW motif-like (HEYL), mRNA”,
gi|19923414|ref|NM_014571.2|[19923414]; 1545: NM_014580, “Homo sapiens solute carrier
family 2, (facilitated glucose transporter) member 8”, “(SLC2A8), mRNA”,
gi|21361448|ref|NM_014580.2|[21361448]; 1546: NM_014581, “Homo sapiens odorant binding
protein 2B (OBP2B), mRNA”, gi|7657406|ref|NM_014581.1|[7657406]; 1547: NM_014588,
“Homo sapiens visual system homeobox 1 homolog, CHX10-like (zebrafish) (VSX1),”,
“transcript variant 1, mRNA”, gi|40806214|ref|NM_014588.4|[40806214]; 1548: NM_014595,
“Homo sapiens 5′,3′-nucleotidase, cytosolic (NT5C), mRNA”,
gi|7657032|ref|NM_014595.1|[7657032]; 1549: NM_014602, “Homo sapiens phosphoinositide-
3-kinase, regulatory subunit 4, p150 (PIK3R4),”, mRNA,
gi|23943911|ref|NM_014602.1|[23943911]; 1550: NM_014606,, ref|NM_014606.1|[7657151],
This record was temporarily removed by RefSeq staff for additional review.,, 1551:
NM_014608, “Homo sapiens cytoplasmic FMR1 interacting protein 1 (CYFIP1), mRNA”,
gi|24307968|ref|NM_014608.1|[24307968]; 1552: NM_014620, “Homo sapiens homeo box C4
(HOXC4), transcript variant 1, mRNA”, gi|24497537|ref|NM_014620.2|[24497537]; 1553:
NM_014621, “Homo sapiens homeo box D4 (HOXD4), mRNA”,
gi|23397671|ref|NM|014621.2|[23397671]; 1554: NM_014623, “Homo sapiens male-enhanced
antigen (MEA), mRNA”, gi|7657325|ref|NM_014623.1|[7657325]; 1555: NM_014625, “Homo
sapiens nephrosis 2, idiopathic, steroid-resistant (podocin) (NPHS2), mRNA”,
gi|7657614|ref|NM_014625.1|[7657614]; 1556: NM_014628, “Homo sapiens MAD2L1 binding
protein (MAD2L1BP), mRNA”, gi|7661917|ref|NM_014628.1|[7661917]; 1557: NM_014632,
“Homo sapiens flavoprotein oxidoreductase MICAL2 (MICAL2), mRNA”,
gi|41281417|ref|NM_014632.2|[41281417]; 1558: NM_014633, Homo sapiens SH2 domain
binding protein 1 (tetratricopeptide repeat containing), “(SH2BP1), mRNA”,
gi|41281407|ref|NM_014633.2|[41281407]; 1559: NM_014652, “Homo sapiens importin 13
(IPO13), mRNA”, gi|41281424|ref|NM_014652.2|[41281424]; 1560: NM_014657, “Homo
sapiens KIAA0406 gene product (KIAA0406), mRNA”,
gi|24307960|ref|NM_014657.1|[24307960]; 1561: NM_014662,, ref|NM_014662.1|[7662221],
This record was temporarily removed by RefSeq staff for additional review.,, 1562:
NM_014671,, ref|NM_014671.1|[7661855], This record was temporarily removed by RefSeq
staff for additional review.,, 1563: NM_014674,, ref|NM_014674.1|[7662001], This record was
temporarily removed by RefSeq staff for additional review.,, 1564: NM_014686,,
ref|NM_014686.1|[7662075], This record was temporarily removed by RefSeq staff for
additional review.,, 1565: NM_014700, “Homo sapiens eferin (Rab11-FIP3), mRNA”,
gi|41281455|ref|NM_014700.2|[41281455]; 1566: NM_014708 “Homo sapiens kinetochore
associated 1 (KNTC1), mRNA”, gi|41327744|ref|NM_014708.3|[41327744]; 1567: NM_014713,
“Homo sapiens lysosomal-associated protein transmembrane 4 alpha (LAPTM4A), mRNA”,
gi|41352690|ref|NM_014713.3|[41352690]; 1568: NM_014714, “Homo sapiens KIAA0590
gene product (KIAA0590), mRNA”, gi|41281446|ref|NM_014714.2|[41281446]; 1569:
NM_014733, “Homo sapiens zinc finger, FYVE domain containing 16 (ZFYVE16), mRNA”,
gi|41281465|ref|NM_014733.2|[41281465]; 1570: NM_014734 “Homo sapiens KIAA0247
(KIAA0247), mRNA”, gi|41281456|ref|NM_014734.2|[41281456]; 1571: NM_014738, “Homo
sapiens KIAA0195 gene product (KIAA0195), mRNA”,
gi|41281472|ref|NM_014738.2|[41281472]; 1572: NM_014748, “Homo sapiens sorting nexin 17
(SNX17), mRNA”, gi|23238249|ref|NM_014748.2|[23238249]; 1573: NM_014753, “Homo
sapiens BMS1-like, ribosome assembly protein (yeast) (BMS1L), mRNA”,
gi|41281482|ref|NM_014753.2|[41281482]; 1574: NM_014754, “Homo sapiens
phosphatidylserine synthase 1 (PTDSS1), mRNA”, gi|7662646|ref|NM_014754.1|[7662646];
1575: NM_014757, “Homo sapiens mastermind-like 1 (Drosophila) (MAML1), mRNA”,
gi|41350321|ref|NM_014757.3|[41350321]; 1576: NM_014760,, ref|NM_014760.1|[7662007],
This record was temporarily removed by RefSeq staff for additional review.,, 1577:
NM_014777,, ref|NM_014777.1|[7661931], This record was temporarily removed by RefSeq
staff for additional review.,, 1578: NM_014783, “Homo sapiens similar to human GTPase-
activating protein (ARHGAP11A), mRNA”, gi|40788020|ref|NM_014783.2|[40788020]; 1579:
NM_014784, “Homo sapiens Rho guanine nucleotide exchange factor (GEF) 11
(ARHGEF11),”, “transcript variant 1, mRNA”, gi|38026914|ref|NM_014784.2|[38026914];
1580: NM_014785,, ref|NM_014785.1|[7662029], This record was temporarily removed by
RefSeq staff for additional review.,, 1581: NM_014786 “Homo sapiens Rho guanine
nucleotide exchange factor (GEF) 17 (ARHGEF17), mRNA”,
gi|21361457|ref|NM_014786.2|[21361457]; 1582: NM_014791, “Homo sapiens maternal
embryonic leucine zipper kinase (MELK), mRNA”, gi|41281490|ref|NM_014791.2|[41281490];
1583: NM_014797,, ref|NM_014797.1|[7662127], This record was temporarily removed by
RefSeq staff for additional review.,, 1584: NM_014805, “Homo sapiens EPM2A (laforin)
interacting protein 1 (EPM2AIP1), mRNA”, gi|31982934|ref|NM_014805.2|[31982934]; 1585:
NM_014813,, ref|NM_014813.1|[7662319], This record was temporarily removed by RefSeq
staff for additional review.,, 1586: NM_014814, “Homo sapiens proteasome regulatory particle
subunit p44S10 (p44S10), mRNA”, gi|7661913|ref|NM_014814.1|[7661913]; 1587: NM_014819,
“Homo sapiens praja 2, RING-H2 motif containing (PJA2), mRNA”,
gi|41281511|ref|NM_014819.2|[41281511]; 1588: NM_014821, “Homo sapiens KIAA0317
(KIAA0317), mRNA”, gi|42734314|ref|NM_014821.2|[42734314]; 1589: NM_014840,,
ref|NM_014840.1|[7662169], This record was temporarily removed by RefSeq staff for
additional review.,, 1590: NM_014845, “Homo sapiens KIAA0274 (KIAA0274), mRNA”,
gi|36030904|ref|NM_014845.4|[36030904]; 1591: NM_014846, “Homo sapiens KIAA0196
gene product (KIAA0196), mRNA”, gi|41281517|ref|NM_014846.2|[41281517]; 1592:
NM_014862, “Homo sapiens aryl-hydrocarbon receptor nuclear translocator 2 (ARNT2),
mRNA”, gi|41281514|ref|NM_014862.2|[41281514]; 1593: NM_014865, “Homo sapiens
chromosome condensation-related SMC-associated protein 1 (CNAP1),”, mRNA,
gi|41281520|ref|NM_014865.2|[41281520]; 1594: NM_014867,, ref|NM_014867.1|[7662259],
This record was temporarily removed by RefSeq staff for additional review.,, 1595:
NM_014872,, ref|NM_014872.1|[7662073], This record was temporarily removed by RefSeq
staff for additional review.,, 1596: NM_014873,, ref|NM_014873.1|[7661995], This record was
temporarily removed by RefSeq staff for additional review.,, 1597: NM_014875,,
ref|NM_014875.1|[7661877], This record was temporarily removed by RefSeq staff for
additional review.,, 1598: NM_014876, “Homo sapiens KIAA0063 gene product (KIAA0063),
mRNA”, gi|34222319|ref|NM_014876.3|[34222319]; 1599: NM_014881, “Homo sapiens DNA
cross-link repair 1A (PSO2 homolog, S. cerevisiae) (DCLRE1A),”, mRNA,
gi|42734318|ref|NM_014881.2|[42734318]; 1600: NM_014886, “Homo sapiens TGF beta-
inducible nuclear protein 1 (TINP1), mRNA”, gi|21359901|ref|NM_014886.2|[21359901]; 1601:
NM_014888, “Homo sapiens family with sequence similarity 3, member C (FAM3C), mRNA”,
gi|7661713|ref|NM_014888.1|[7661713]; 1602: NM_014889, “Homo sapiens pitrilysin
metalloproteinase 1 (PITRM1), mRNA”, gi|41352060|ref|NM_014889.2|[41352060]; 1603:
NM_014892,, ref|NM_014892.1|[7662491], This record was temporarily removed by RefSeq
staff for additional review.,, 1604: NM_014901, “Homo sapiens ring finger protein 44
(RNF44), mRNA”, gi|42718018|ref|NM_014901.4|[42718018]; 1605: NM_014907, “Homo
sapiens FERM and PDZ domain containing 1 (FRMPD1), mRNA”,
gi|7662415|ref|NM_014907.1|[7662415]; 1606: NM_014910,, ref|NM_014910.1|[7662479],
This record was temporarily removed by RefSeq staff for additional review.,, 1607:
NM_014914, “Homo sapiens centaurin, gamma 2 (CENTG2), mRNA”,
gi|41281554|ref|NM_014914.2|[41281554]; 1608: NM_014917,, ref|NM_014917.1|[7662425],
This record was temporarily removed by RefSeq staff for additional review.,, 1609:
NM_014935, “Homo sapiens phosphoinositol 3-phosphate-binding protein-3 (PEPP3), mRNA”,
gi|37595547|ref|NM_014935.2|[37595547]; 1610: NM_014937, “Homo sapiens inositol
polyphosphate-5-phosphatase F (INPP5F), transcript variant”, “1, mRNA”,
gi|38327540|ref|NM_014937.2|[38327540]; 1611: NM_014939, “Homo sapiens KIAA1012
(KIAA1012), mRNA”, gi|42476075|ref|NM_014939.2|[42476075]; 1612: NM_014940, “Homo
sapiens HSV-1 stimulation-related gene 1 (HSRG1), mRNA”,
gi|38016939|ref|NM_014940.2|[38016939]; 1613: NM_014949,, ref|NM_014949.1|[7662371],
This record was temporarily removed by RefSeq staff for additional review.,, 1614:
NM_014977, “Homo sapiens apoptotic chromatin condensation inducer in the nucleus
(ACINUS),”, mRNA, gi|7662237|ref|NM_014977.1|[7662237]; 1615: NM_014992, “Homo
sapiens dishevelled associated activator of morphogenesis 1 (DAAM1), mRNA”,
gi|21071076|ref|NM_014992.1|[21071076]; 1616: NM_015029, “Homo sapiens processing of
precursors 1 (POP1), mRNA”, gi|23097291|ref|NM_015029.1|[23097291]; 1617: NM_015039,
“Homo sapiens nicotinamide nucleotide adenylyltransferase 2 (NMNAT2), transcript”, “variant
1, mRNA”, gi|25141321|ref|NM_015039.2|[25141321]; 1618: NM_015050, “Homo sapiens
KIAA0082 (KIAA0082), mRNA”, gi|24307982|ref|NM_015050.1|[24307982]; 1619:
NM_015064, “Homo sapiens Rab6-interacting protein 2 (ELKS), transcript variant alpha,
mRNA”, gi|38045899|ref|NM_015064.2|[38045899]; 1620: NM_015074, “Homo sapiens
kinesin family member 1B (KIF1B), transcript variant 1, mRNA”,
gi|41393562|ref|NM_015074.2|[41393562]; 1621: NM_015078, “Homo sapiens Rho family
guanine-nucleotide exchange factor (KIAA0861), mRNA”,
gi|31742504|ref|NM_015078.2|[31742504]; 1622: NM_015089, “Homo sapiens p53-associated
parkin-like cytoplasmic protein (PARC), mRNA”, gi|24307990|ref|NM_015089.1|[24307990];
1623: NM_015101, “Homo sapiens chromosome 1 open reading frame 17 (C1orf17), mRNA”,
gi|16506819|ref|NM_015101.1|[16506819]; 1624: NM_015149, “Homo sapiens RalGDS-like
gene (RGL), mRNA”, gi|20127535|ref|NM_015149.2|[20127535]; 1625: NM_015163, “Homo
sapiens tripartite motif-containing 9 (TRIM9), transcript variant 1, mRNA”,
gi|29543553|ref|NM_015163.3|[29543553]; 1626: NM_015169, “Homo sapiens RRS1 ribosome
biogenesis regulator homolog (S. cerevisiae) (RRS1),”, mRNA,
gi|34147329|ref|NM_015169.2|[34147329]; 1627: NM_015178, “Homo sapiens Rho-related
BTB domain containing 2 (RHOBTB2), mRNA”, gi|14165285|ref|NM_015178.1|[14165285];
1628: NM_015198, “Homo sapiens cordon-bleu homolog (mouse) (COBL), mRNA”,
gi|31581523|ref|NM_015198.2|[31581523]; 1629: NM_015216, “Homo sapiens KIAA0433
protein (KIAA0433), mRNA”, gi|41281582|ref|NM_015216.2|[41281582]; 1630: NM_015254,
“Homo sapiens kinesin family member 13B (KIF13B), mRNA”,
gi|13194196|ref|NM_015254.1|[13194196]; 1631: NM_015292, Homo sapiens likely ortholog
of mouse membrane bound C2 domain containing, “protein (MBC2), mRNA”,
gi|14149679|ref|NM_015292.1|[14149679]; 1632: NM_015308, “Homo sapiens formin binding
protein 4 (FNBP4), mRNA”, gi|24308032|ref|NM_015308.1|[24308032]; 1633: NM_015316,
“Homo sapiens protein phosphatase 1, regulatory (inhibitor) subunit 13B”, “(PPP1R13B),
mRNA”, gi|18699719|ref|NM_015316.1|[18699719]; 1634: NM_015318, “Homo sapiens
rho/rac guanine nucleotide exchange factor (GEF) 18 (ARHGEF18),”, mRNA,
gi|41327768|ref|NM_015318.2|[41327768]; 1635: NM_015331, “Homo sapiens nicastrin
(NCSTN), mRNA”, gi|24638432|ref|NM_015331.1|[24638432]; 1636: NM_015339, “Homo
sapiens activity-dependent neuroprotector (ADNP), transcript variant 1,”, mRNA,
gi|31563504|ref|NM_015339.2|[31563504]; 1637: NM_015341, “Homo sapiens barren homolog
(Drosophila) (BRRN1), mRNA”, gi|25121986|ref|NM_015341.2|[25121986]; 1638: NM_015343,
“Homo sapiens dullard homolog (Xenopus laevis) (DULLARD), mRNA”,
gi|34222318|ref|NM_015343.3|[34222318]; 1639: NM_015358, “Homo sapiens zinc finger,
CW-type with coiled-coil domain 3 (ZCWCC3), mRNA”,
gi|28872811|ref|NM_015358.1|[28872811]; 1640: NM_015362,, ref|NM_015362.3|[44662829];
1641: NM_015368, “Homo sapiens pannexin 1 (PANX1), mRNA”,
gi|39995063|ref|NM_015368.3|[39995063]; 1642: NM_015372, “Homo sapiens hypothetical
protein HSN44A4A (HSN44A4A), mRNA”, gi|7661723|ref|NM_015372.1|[7661723]; 1643:
NM_015376,, ref|NM_015376.1|[7662333], This record was temporarily removed by RefSeq
staff for additional review.,, 1644: NM_015388, “Homo sapiens chromosome 6 open reading
frame 109 (C6orf109), mRNA”, gi|7661641|ref|NM_015388.1|[7661641]; 1645: NM_015393,
“Homo sapiens DKFZP564O0823 protein (DKFZP564O0823), mRNA”,
gi|7661631|ref|NM_015393.1|[7661631]; 1646: NM_015399, “Homo sapiens breast cancer
metastasis suppressor 1 (BRMS1), mRNA”, gi|24475631|ref|NM_015399.2|[24475631]; 1647:
NM_015407, “Homo sapiens DKFZP564O243 protein (DKFZP564O243), mRNA”,
gi|34147328|ref|NM_015407.3|[34147328]; 1648: NM_015414, “Homo sapiens ribosomal
protein L36 (RPL36), transcript variant 2, mRNA”, gi|16117793|ref|NM_015414.2|[16117793];
1649: NM_015416, “Homo sapiens cervical cancer 1 protooncogene (HCCR1), mRNA”,
gi|21166356|ref|NM_015416.2|[21166356]; 1650: NM_015439, “Homo sapiens chromosome 6
open reading frame 80 (C6orf80), mRNA”, gi|31083115|ref|NM_015439.2|[31083115]; 1651:
NM_015480, “Homo sapiens poliovirus receptor-related 3 (PVRL3), mRNA”,
gi|11386198|ref|NM_015480.1|[11386198]; 1652: NM_015484, “Homo sapiens GCIP-
interacting protein p29 (P29), mRNA”, gi|7661635|ref|NM_015484.1|[7661635]; 1653:
NM_015485, “Homo sapiens RWD domain containing 3 (RWDD3), mRNA”,
gi|21361481|ref|NM_015485.2|[21361481]; 1654: NM_015490, “Homo sapiens SEC31-like 2
(S. cerevisiae) (SEC31L2), transcript variant 1, mRNA”,
gi|38149839|ref|NM_015490.3|[38149839]; 1655: NM_015509, “Homo sapiens
DKFZP566B183 protein (DKFZP566B183), mRNA”,
gi|31542527|ref|NM_015509.2|[31542527]; 1656: NM_015510, “Homo sapiens
DKFZP566O084 protein (DKFZp566O084), mRNA”,
gi|23065521|ref|NM_015510.3|[23065521]; 1657: NM_015511, “Homo sapiens chromosome 20
open reading frame 4 (C20orf4), mRNA”, gi|18034689|ref|NM_015511.2|[18034689]; 1658:
NM_015513, “Homo sapiens cysteine-rich with EGF-like domains 1 (CRELD1), mRNA”,
gi|22095396|ref|NM_015513.2|[22095396]; 1659: NM_015517, Homo sapiens MBD2 (methyl-
CpG-binding protein)-interacting zinc finger protein, “(MIZF), transcript variant 1, mRNA”,
gi|39725947|ref|NM_015517.3|[39725947]; 1660: NM_015527, “Homo sapiens
DKFZP434P1750 protein (DKFZP434P1750), mRNA”,
gi|21361484|ref|NM_015527.2|[21361484]; 1661: NM_015533, “Horno sapiens
DKFZP586B1621 protein (DKFZP586B1621), mRNA”,
gi|20149620|ref|NM_015533.2|[20149620]; 1662: NM_015535, “Homo sapiens DNA
polymerase-transactivated protein 6 (DNAPTP6), mRNA”,
gi|7661597|ref|NM_015535.1|[7661597]; 1663: NM_015540, “Homo sapiens DKFZP727M111
protein (DKFZP727M111), mRNA”, gi|24430138|ref|NM_015540.2|[24430138]; 1664:
NM_015558, Homo sapiens synovial sarcoma translocation gene on chromosome 18-like 1,
“(SS18L1), transcript variant 2, mRNA”, gi|39777611|ref|NM_015558.3|[39777611]; 1665:
NM_015570, “Homo sapiens autism susceptibility candidate 2 (AUTS2), mRNA”,
gi|17864089|ref|NM_015570.1|[17864089]; 1666: NM_015582, “Homo sapiens
DKFZP564B147 protein (DKFZP564B147), mRNA”, gi|7661599|ref|NM_015582.1|[7661599];
1667: NM_015584, “Homo sapiens polymerase (DNA-directed), delta interacting protein 2
(POLDIP2),”, mRNA, gi|30089946|ref|NM_015584.2|[30089946]; 1668: NM_015603, “Homo
sapiens coiled-coil domain containing 9 (CCDC9), mRNA”,
gi|7661689|ref|NM_015603.1|[7661689]; 1669: NM_015604, “Homo sapiens WD repeat
domain 21 (WDR21), transcript variant 1, mRNA”, gi|31317287|ref|NM_015604.2|[31317287];
1670: NM_015623,, ref|NM_015623.2|[32306520], This record was temporarily removed by
RefSeq staff for additional review.,, 1671: NM_015646, “Homo sapiens RAP1B, member of
RAS oncogene family (RAP1B), mRNA”, gi|34222320|ref|NM_015646.3|[34222320]; 1672:
NM_015649, “Homo sapiens interferon regulatory factor 2 binding protein 1 (IRF2BP1),
mRNA”, gi|24308114|ref|NM_015649.1|[24308114]; 1673: NM_015653, “Homo sapiens
DKFZP566F0546 protein (DKFZP566F0546), mRNA”,
gi|13124762|ref|NM_015653.1|[13124762]; 1674: NM_015654, “Homo sapiens
DKFZP564C103 protein (DKFZP564C103), mRNA”,
gi|34222325|ref|NM_015654.3|[34222325]; 1675: NM_015691, “Homo sapiens KIAA1280
protein (KIAA1280), mRNA”, gi|38570148|ref|NM_015691.2|[38570148]; 1676: NM_015699,,
ref|NM_015699.1|[7661559], This record was temporarily removed by RefSeq staff for
additional review.,, 1677: NM_015702, “Homo sapiens hypothetical protein CL25022
(CL25022), mRNA”, gi|7661547|ref|NM_015702.1|[7661547]; 1678: NM_015710, “Homo
sapiens glioma tumor suppressor candidate region gene 2 (GLTSCR2), mRNA”,
gi|21359905|ref|NM_015710.2|[21359905]; 1679: NM_015714, “Homo sapiens putative
lymphocyte G0/G1 switch gene (G0S2), mRNA”, gi|20070269|ref|NM_015714.2|[20070269];
1680: NM_015715, “Homo sapiens phospholipase A2, group III (PLA2G3), mRNA”,
gi|7657125|ref|NM_015715.1|[7657125]; 1681: NM_015722, “Homo sapiens calcyon protein
(CALCYON), mRNA”, gi|9257200|ref|NM_015722.2|[9257200]; 1682: NM_015855, “Homo
sapiens Wilms tumor associated protein (WIT-1), mRNA”,
gi|19743572|ref|NM_015855.2|[19743572]; 1683: NM_015859, “Homo sapiens general
transcription factor IIA, 1, 19/37 kDa (GTF2A1), transcript”, “variant 1, mRNA”,
gi|42476103|ref|NM_015859.2|[42476103]; 1684: NM_015865, “Homo sapiens solute carrier
family 14 (urea transporter), member 1 (Kidd blood”, “group) (SLC14A1), mRNA”,
gi|7706676|ref|NM_015865.1|[7706676]; 1685: NM_015871, “Homo sapiens zinc finger protein
(ZT86), mRNA”, gi|21359908|ref|NM_015871.2|[21359908]; 1686: NM_015884, “Homo
sapiens membrane-bound transcription factor protease, site 2 (MBTPS2), mRNA”,
gi|7706692|ref|NM_015884.1|[7706692]; 1687: NM_015885, “Homo sapiens pre-mRNA
cleavage complex II protein Pcf11 (PCF11), mRNA”,
gi|33620744|ref|NM_015885.2|[33620744]; 1688: NM_015889, “Homo sapiens PC2 (positive
cofactor 2, multiprotein complex)”, “glutamine/Q-rich-associated protein (PCQAP), mRNA”,
gi|21312133|ref|NM_015889.2|[21312133]; 1689: NM_015894, “Homo sapiens stathmin-like 3
(STMN3), mRNA”, gi|14670374|ref|NM_015894.2|[14670374]; 1690: NM_015895, “Homo
sapiens geminin, DNA replication inhibitor (GMNN), mRNA”,
gi|41393571|ref|NM_015895.3|[41393571]; 1691: NM_015901, Homo sapiens nudix
(nucleoside diphosphate linked moiety X)-type motif 13, “(NUDT13), mRNA”,
gi|34330151|ref|NM_015901.3|[34330151]; 1692: NM_015918, “Homo sapiens RNase
MRP/RNase P protein-like (POP5), transcript variant 1, mRNA”,
gi|38016924|ref|NM_015918.3|[38016924]; 1693: NM_015920, “Homo sapiens ribosomal
protein S27-like (RPS27L), mRNA”, gi|18490988|ref|NM_015920.2|[18490988]; 1694:
NM_015921, “Homo sapiens chromosome 6 open reading frame 82 (C6orf82), mRNA”,
gi|7706243|ref|NM_015921.1|[7706243]; 1695: NM_015925, “Homo sapiens liver-specific
bHLH-Zip transcription factor (LISCH7), mRNA”, gi|34916060|ref|NM_015925.3|[34916060];
1696: NM_015926, “Homo sapiens putative secreted protein ZSIG11 (ZSIG11), mRNA”,
gi|34147580|ref|NM_015926.3|[34147580]; 1697: NM_015927, “Homo sapiens transforming
growth factor beta 1 induced transcript 1 (TGFB1I1),”, mRNA,
gi|34147679|ref|NM_015927.3|[34147679]; 1698: NM_015929, “Homo sapiens
lipoyltransferase 1 (LIPT1), transcript variant 1, mRNA”,
gi|21729874|ref|NM_015929.2|[21729874]; 1699: NM_015932, “Homo sapiens chromosome 13
open reading frame 12 (C13orf12), mRNA”, gi|21361533|ref|NM_015932.2|[21361533]; 1700:
NM_015937, “Homo sapiens phosphatidylinositol glycan, class T (PIGT), mRNA”,
gi|23397652|ref|NM_015937.2|[23397652]; 1701: NM_015938, “Homo sapiens CGI-07 protein
(CGI-07), mRNA”, gi|19923795|ref|NM_015938.2|[19923795]; 1702: NM_015941, “Homo
sapiens ATPase, H+ transporting, lysosomal 50/57 kD V1 subunit H (ATP6V1H),”, mRNA,
gi|7706261|ref|NM_015941.1|[7706261]; 1703: NM_015942, “Homo sapiens CGI-12 protein
(CGI-12), mRNA”, gi|34147675|ref|NM_015942.3|[34147675]; 1704: NM_015945, “Homo
sapiens solute carrier family 35, member C2 (SLC35C2), transcript variant”, “2, mRNA”,
gi|34335287|ref|NM_015945.10|[34335287]; 1705: NM_015947, “Homo sapiens CGI-18
protein (CGI-18), mRNA”, gi|7705601|ref|NM_015947.1|[7705601]; 1706: NM_015950,
“Homo sapiens mitochondrial ribosomal protein L2 (MRPL2), nuclear gene encoding”,
“mitochondrial protein, mRNA”, gi|41872659|ref|NM_015950.3|[41872659]; 1707: NM_015953
“Homo sapiens nitric oxide synthase interacting protein (NOSIP), mRNA”,
gi|34147607|ref|NM_015953.3|[34147607]; 1708: NM_015956, “Homo sapiens mitochondrial
ribosomal protein L4 (MRPL4), nuclear gene encoding”, “mitochondrial protein, transcript
variant 1, mRNA”, gi|22547135|ref|NM_015956.2|[22547135]; 1709: NM_015959, “Homo
sapiens thioredoxin-related transmembrane protein 2 (TMX2), mRNA”,
gi|7705725|ref|NM_015959.1|[7705725]; 1710: NM_015960, “Homo sapiens CGI-32 protein
(CGI-32), mRNA”, gi|7705727|ref|NM_015960.1|[7705727]; 1711: NM_015962, “Homo
sapiens chromosome 14 open reading frame 111 (C14orf111), mRNA”,
gi|7705729|ref|NM_015962.1|[7705729]; 1712: NM_015964, “Homo sapiens brain specific
protein (CGI-38), mRNA”, gi|7706275|ref|NM_015964.1|[7706275]; 1713: NM_015965,
“Homo sapiens cell death-regulatory protein GRIM19 (GRIM19), mRNA”,
gi|21361821|ref|NM_015965.3|[21361821]; 1714: NM_015971, “Homo sapiens mitochondrial
ribosomal protein S7 (MRPS7), nuclear gene encoding”, “mitochondrial protein, mRNA”,
gi|16554617|ref|NM_015971.2|[16554617]; 1715: NM_015972, “Homo sapiens polymerase
(RNA) I polypeptide D, 16 kDa (POLR1D), mRNA”, gi|7705739|ref|NM_015972.1|[7705739];
1716: NM_015974, “Homo sapiens crystallin, lambda 1 (CRYL1), mRNA”,
gi|7705743|ref|NM_015974.1|[7705743]; 1717: NM_015976, “Homo sapiens sorting nexin 7
(SNX7), transcript variant 1, mRNA”, gi|23111053|ref|NM_015976.2|[23111053]; 1718:
NM_015982, “Homo sapiens germ cell specific Y-box binding protein (YBX2), mRNA”,
gi|7705750|ref|NM_015982.1|[7705750]; 1719: NM_015986, “Homo sapiens cytokine receptor-
like factor 3 (CRLF3), mRNA”, gi|27764872|ref|NM_015986.2|[27764872]; 1720: NM_015991,
“Homo sapiens complement component 1, q subcomponent, alpha polypeptide (C1QA),”,
mRNA, gi|7705752|ref|NM_015991.1|[7705752]; 1721: NM_015997, “Homo sapiens CGI-41
protein (CGI-41), mRNA”, gi|21361524|ref|NM_015997.2|[21361524]; 1722: NM_015999,
“Homo sapiens adiponectin receptor 1 (ADIPOR1), mRNA”,
gi|21361518|ref|NM_015999.2|[21361518]; 1723: NM_016004, “Homo sapiens chromosome 20
open reading frame 9 (C20orf9), mRNA”, gi|7705768|ref|NM_016004.1|[7705768]; 1724:
NM_016011, “Homo sapiens nuclear receptor binding factor 1 (CGI-63), mRNA”,
gi|7705776|ref|NM_016011.1|[7705776]; 1725: NM_016013, “Homo sapiens NADH
dehydrogenase (ubiquinone) 1 alpha subcomplex, assembly factor”, “1 (NDUFAF1), mRNA”,
gi|7705778|ref|NM_016013.1|[7705778]; 1726: NM_016015, “Homo sapiens leucine carboxyl
methyltransferase 1 (LCMT1), mRNA”, gi|15082255|ref|NM_016015.2|[15082255]; 1727:
NM_016020, “Homo sapiens transcription factor B1, mitochondrial (TFB1M), mRNA”,
gi|7705784|ref|NM_016020.1|[7705784]; 1728: NM_016022, Homo sapiens likely ortholog of
C. elegans anterior pharynx defective 1A, “(APH-1A), mRNA”,
gi|7705786|ref|NM_016022.1|[7705786]; 1729: NM_016027, “Homo sapiens lactamase, beta 2
(LACTB2), mRNA”, gi|7705792|ref|NM_016027.1|[7705792]; 1730: NM_016028, “Homo
sapiens CGI-85 protein (CGI-85), transcript variant 2, mRNA”,
gi|27477098|ref|NM_016028.3|[27477098]; 1731: NM_016033, “Homo sapiens CGI-90 protein
(CGI-90), mRNA”, gi|7705802|ref|NM_016033.1|[7705802]; 1732: NM_016045, “Homo
sapiens chromosome 20 open reading frame 45 (C20orf45), mRNA”,
gi|7705609|ref|NM_016045.1|[7705609]; 1733: NM_016046, “Homo sapiens exosomal core
protein CSL4 (CSL4), mRNA”, gi|22035626|ref|NM_016046.2|[22035626]; 1734: NM_016049,
“Homo sapiens chromosome 14 open reading frame 122 (C14orf122), mRNA”,
gi|34222327|ref|NM_016049.3|[34222327]; 1735: NM_016052, “Homo sapiens CGI-115
protein (CGI-115), mRNA”, gi|31542299|ref|NM_016052.2|[31542299]; 1736: NM_016053,
“Homo sapiens CGI-116 protein (CGI-116), mRNA”, gi|7705621|ref|NM_016053.1|[7705621];
1737: NM_016055, “Homo sapiens mitochondrial ribosomal protein L48 (MRPL48), nuclear
gene encoding”, “mitochondrial protein, mRNA”, gi|38788229|ref|NM_016055.3|[38788229];
1738: NM_016056, “Homo sapiens CGI-119 protein (CGI-119), mRNA”,
gi|7706334|ref|NM_016056.1|[7706334]; 1739: NM_016062, “Homo sapiens CGI-128 protein
(CGI-128), mRNA”, gi|7706342|ref|NM_016062.1|[7706342]; 1740: NM_016065, “Homo
sapiens mitochondrial ribosomal protein S16 (MRPS16), nuclear gene encoding”, “mitochondrial
protein, mRNA”, gi|16554612|ref|NM_016065.2|[16554612]; 1741: NM_016067, “Homo
sapiens mitochondrial ribosomal protein S18C (MRPS18C), nuclear gene”, “encoding
mitochondrial protein, mRNA”, gi|7705629|ref|NM_016067.1|[7705629]; 1742: NM_016069,
Homo sapiens mitochondria-associated protein involved in granulocyte-macrophage, “colony-
stimulating factor signal transduction (Magmas), nuclear gene encoding”, “mitochondrial
protein, mRNA”, gi|27363460|ref|NM_016069.8|[27363460]; 1743: NM_016071, “Homo
sapiens mitochondrial ribosomal protein S33 (MRPS33), nuclear gene encoding”, “mitochondrial
protein, transcript variant 1, mRNA”, gi|16950595|ref|NM_016071.2|[16950595]; 1744:
NM_016072, “Homo sapiens CGI-141 protein (CGI-141), mRNA”,
gi|19923443|ref|NM_016072.2|[19923443]; 1745: NM_016079, “Homo sapiens neuroendocrine
differentiation factor (NEDF), mRNA”, gi|7706352|ref|NM_016079.1|[7706352]; 1746:
NM_016080, “Homo sapiens CGI-150 protein (CGI-150), mRNA”,
gi|34850073|ref|NM_016080.2|[34850073]; 1747: NM_016082, “Homo sapiens CDK5
regulatory subunit associated protein 1 (CDK5RAP1), transcript”, “variant 2, mRNA”,
gi|28872783|ref|NM_016082.3|[28872783]; 1748: NM_016086, “Homo sapiens map kinase
phosphatase-like protein MK-STYX (MK-STYX), mRNA”,
gi|32481212|ref|NM_016086.2|[32481212]; 1749: NM_016087, “Homo sapiens wingless-type
MMTV integration site family, member 16 (WNT16),”, “transcript variant 2, mRNA”,
gi|17402913|ref|NM_016087.2|[17402913]; 1750: NM_016090, “Homo sapiens RNA binding
motif protein 7 (RBM7), mRNA”, gi|31543547|ref|NM_016090.2|[31543547]; 1751:
NM_016091, “Homo sapiens eukaryotic translation initiation factor 3, subunit 6 interacting”,
“protein (EIF3S6IP), mRNA”, gi|7705432|ref|NM_016091.1|[7705432]; 1752: NM_016095,
“Homo sapiens DNA replication complex GINS protein PSF2 (Pfs2), mRNA”,
gi|7706366|ref|NM_016095.1|[7706366]; 1753: NM_016097, “Homo sapiens HSPC039 protein
(HSPC039), mRNA”, gi|32261311|ref|NM_016097.2|[32261311]; 1754: NM_016099, “Homo
sapiens golgi autoantigen, golgin subfamily a, 7 (GOLGA7), mRNA”,
gi|7705820|ref|NM_016099.1|[7705820]; 1755: NM_016101, “Homo sapiens comparative gene
identification transcript 37 (CGI-37), mRNA”, gi|40538791|ref|NM_016101.2|[40538791]; 1756:
NM_016102, “Homo sapiens tripartite motif-containing 17 (TRIM17), mRNA”,
gi|7705824|ref|NM_016102.1|[7705824]; 1757: NM_016103, “Homo sapiens SAR1a gene
homolog 2 (S. cerevisiae) (SARA2), mRNA”, gi|38176155|ref|NM_016103.2|[38176155]; 1758:
NM_016106, “Homo sapiens sec1 family domain containing 1 (SCFD1), transcript variant 1,
mRNA”, gi|33469965|ref|NM_016106.2|[33469965]; 1759: NM_016127, “Homo sapiens
hypothetical protein MGC8721 (MGC8721), mRNA”,
gi|42476192|ref|NM_016127.4|[42476192]; 1760: NM_016133, “Homo sapiens insulin induced
gene 2 (INSIG2), mRNA”, gi|38327532|ref|NM_016133.2|[38327532]; 1761: NM_016139,
“Homo sapiens coiled-coil-helix-coiled-coil-helix domain containing 2 (CHCHD2),”, mRNA,
gi|32307179|ref|NM_016139.2|[32307179]; 1762: NM_016142, “Homo sapiens hydroxysteroid
(17-beta) dehydrogenase 12 (HSD17B12), mRNA”, gi|7705854|ref|NM_016142.1|[7705854];
1763: NM_016145, “Homo sapiens PTD008 protein (PTD008), mRNA”,
gi|7706664|ref|NM_016145.1|[7706664]; 1764: NM_016148, “Homo sapiens SH3 and multiple
ankyrin repeat domains 1 (SHANK1), mRNA”, gi|11968151|ref|NM_016148.1|[11968151];
1765: NM_016158, “Homo sapiens erythrocyte transmembrane protein (LOC51145), mRNA”,
gi|7705856|ref|NM_016158.1|[7705856]; 1766: NM_016183, “Homo sapiens chromosome 1
open reading frame 33 (Clorf33), mRNA”, gi|18490986|ref|NM_016183.2|[18490986]; 1767:
NM_016185, “Homo sapiens hematological and neurological expressed 1 (HN1), mRNA”,
gi|7705876|ref|NM_016185.1|[7705876]; 1768: NM_016187, “Homo sapiens bridging
integrator 2 (BIN2), mRNA”, gi|7705295|ref|NM_016187.1|[7705295]; 1769: NM_016195,
“Homo sapiens M-phase phosphoprotein 1 (MPHOSPH1), mRNA”,
gi|7705347|ref|NM_016195.1|[7705347]; 1770: NM_016200, “Homo sapiens LSM8 homolog,
U6 small nuclear RNA associated (S. cerevisiae)”, “(LSM8), mRNA”,
gi|21314665|ref|NM_016200.2|[21314665]; 1771: NM_016202, “Homo sapiens zinc finger
protein 580 (ZNF580), mRNA”, gi|7705880|ref|NM_016202.1|[7705880]; 1772: NM_016203,
“Homo sapiens protein kinase, AMP-activated, gamma 2 non-catalytic subunit”, “(PRKAG2),
mRNA”, gi|33186924|ref|NM_016203.2|[33186924]; 1773: NM_016206, “Homo sapiens colon
carcinoma related protein (LOC51159), mRNA”, gi|7705882|ref|NM_016206.1|[7705882]; 1774:
NM_016209, “Homo sapiens unknown (LOC51693), mRNA”,
gi|7706428|ref|NM_016209.1|[7706428]; 1775: NM_016210, “Homo sapiens g20 protein
(LOC51161), mRNA”, gi|31543080|ref|NM_016210.2|[31543080]; 1776: NM_016216, “Homo
sapiens debranching enzyme homolog 1 (S. cerevisiae) (DBR1), mRNA”,
gi|7705890|ref|NM_016216.1|[7705890]; 1777: NM_016223, Homo sapiens protein kinase C
and casein kinase substrate in neurons 3, “(PACSIN3), mRNA”,
gi|34147484|ref|NM_016223.3|[34147484]; 1778: NM_016229, “Homo sapiens cytochrome b5
reductase b5R.2 (CYB5R2), mRNA”, gi|7706442|ref|NM_016229.1|[7706442]; 1779:
NM_016230, “Homo sapiens NADPH cytochrome B5 oxidoreductase (NCB5OR), mRNA”,
gi|21314659|ref|NM_016230.2|[21314659]; 1780: NM_016231, “Homo sapiens nemo like
kinase (NLK), mRNA”, gi|42734431|ref|NM_016231.2|[42734431]; 1781: NM_016245, “Homo
sapiens dehydrogenase/reductase (SDR family) member 8 (DHRS8), mRNA”,
gi|7705904|ref|NM_016245.1|[7705904]; 1782: NM_016246, “Homo sapiens
dehydrogenase/reductase (SDR family) member 10 (DHRS10), mRNA”,
gi|7705906|ref|NM_016246.1|[7705906]; 1783: NM_016255, “Homo sapiens family with
sequence similarity 8, member A1 (FAM8A1), mRNA”,
gi|7705267|ref|NM_016255.1|[7705267]; 1784: NM_016256, Homo sapiens N-
acetylglucosamine-1-phosphodiester alpha-N-acetylglucosaminidase, “(NAGPA), mRNA”,
gi|7705908|ref|NM_016256.1|[7705908]; 1785: NM_016258, “Homo sapiens high-glucose-
regulated protein 8 (HGRG8), mRNA”, gi|7705410|ref|NM_016258.1|[7705410]; 1786:
NM_016260, “Homo sapiens zinc finger protein, subfamily 1A, 2 (Helios) (ZNFN1A2),
mRNA”, gi|7705910|ref|NM_016260.1|[7705910]; 1787: NM_016265, “Homo sapiens zinc
finger protein 325 (ZNF325), mRNA”, gi|7706464|ref|NM_016265.1|[7706464]; 1788:
NM_016286, “Homo sapiens dicarbonyl/L-xylulose reductase (DCXR), mRNA”,
gi|41350203|ref|NM_016286.2|[41350203]; 1789: NM_016287, “Homo sapiens HP1-BP74
(HP1-BP74), mRNA”, gi|7705416|ref|NM_016287.1|[7705416]; 1790: NM_016289, “Homo
sapiens MO25 protein (MO25), mRNA”, gi|19745179|ref|NM_016289.2|[19745179]; 1791:
NM_016304, “Homo sapiens chromosome 15 open reading frame 15 (C15orf15), mRNA”,
gi|18491027|ref|NM_016304.2|[18491027]; 1792: NM_016308, “Homo sapiens UMP-CMP
kinase (UMP-CMPK), mRNA”, gi|7706496|ref|NM_016308.1|[7706496]; 1793: NM_016310,
“Homo sapiens polymerase (RNA) III (DNA directed) polypeptide K, 12.3 kDa”, “(POLR3K),
mRNA”, gi|14589957|ref|NM_016310.2|[14589957]; 1794: NM_016316, “Homo sapiens
REV1-like (yeast) (REV1L), mRNA”, gi|7706680|ref|NM_016316.1|[7706680]; 1795:
NM_016319, Homo sapiens COP9 constitutive photomorphogenic homolog subunit 7A
(Arabidopsis), “(COPS7A), mRNA”, gi|7705329|ref|NM_016319.1|[7705329]; 1796:
NM_016324, “Homo sapiens zinc finger protein 274 (ZNF274), transcript variant ZNF274b,
mRNA”, gi|19743797|ref|NM_016324.2|[19743797]; 1797: NM_016332, “Homo sapiens
selenoprotein X, 1 (SEPX1), mRNA”, gi|7706510|ref|NM_016332.1|[7706510]; 1798:
NM_016337, “Homo sapiens Enah/Vasp-like (EVL), mRNA”,
gi|7706686|ref|NM_016337.1|[7706686]; 1799: NM_016354, “Homo sapiens solute carrier
organic anion transporter family, member 4A1”, “(SLCO4A1), mRNA”,
gi|39777593|ref|NM_016354.3|[39777593]; 1800: NM_016355, “Homo sapiens DEAD (Asp-
Glu-Ala-Asp) box polypeptide 47 (DDX47), transcript”, “variant 1, mRNA”,
gi|41327774|ref|NM_016355.3|[41327774]; 1801: NM_016358, “Homo sapiens iroquois
homeobox protein 4 (IRX4), mRNA”, gi|7705554|ref|NM_016358.1|[7705554]; 1802:
NM_016364, “Homo sapiens dual specificity phosphatase 13 (DUSP13), mRNA”,
gi|20149630|ref|NM_016364.2|[20149630]; 1803: NM_016368, “Homo sapiens myo-inositol 1-
phosphate synthase A1 (ISYNA1), mRNA”, gi|21902536|ref|NM_016368.3|[21902536]; 1804:
NM_016371, “Homo sapiens hydroxysteroid (17-beta) dehydrogenase 7 (HSD17B7), mRNA”,
gi|7705420|ref|NM_016371.1|[7705420]; 1805: NM_016372, “Homo sapiens seven
transmembrane domain orphan receptor (TPRA40), mRNA”,
gi|7705964|ref|NM_016372.1|[7705964]; 1806: NM_016397, “Homo sapiens TH1-like
(Drosophila) (TH1L), transcript variant 2, mRNA”, gi|39812483|ref|NM_016397.2|[39812483];
1807: NM_016400, “Homo sapiens Huntingtin interacting protein K (HYPK), mRNA”,
gi|21361540|ref|NM_016400.2|[21361540]; 1808: NM_016404, “Homo sapiens hypothetical
protein HSPC152 (HSPC152), mRNA”, gi|7705476|ref|NM_016404.1|[7705476]; 1809:
NM_016406, “Homo sapiens hypothetical protein HSPC155 (HSPC155), mRNA”,
gi|7705480|ref|NM_016406.1|[7705480]; 1810: NM_016407, “Homo sapiens chromosome 20
open reading frame 43 (C20orf43), mRNA”, gi|7705482|ref|NM_016407.1|[7705482]; 1811:
NM_016412, “Homo sapiens insulin-like growth factor 2, antisense (IGF2AS), mRNA”,
gi|7705972|ref|NM_016412.1|[7705972]; 1812: NM_016422, “Homo sapiens ring finger protein
141 (RNF141), mRNA”, gi|38045936|ref|NM_016422.3|[38045936]; 1813: NM_016423,
“Homo sapiens zinc finger protein 219 (ZNF219), mRNA”,
gi|7705974|ref|NM_016423.1|[7705974]; 1814: NM_016433, “Homo sapiens glycolipid transfer
protein (GLTP), mRNA”, gi|20357594|ref|NM_016433.2|[20357594]; 1815: NM_016447,
“Homo sapiens membrane protein, palmitoylated 6 (MAGUK p55 subfamily member 6)”,
“(MPP6), mRNA”, gi|21361597|ref|NM_016447.2|[21361597]; 1816: NM_016448, “Homo
sapiens RA-regulated nuclear matrix-associated protein (RAMP), mRNA”,
gi|7705575|ref|NM_016448.1|[7705575]; 1817: NM_016453, “Homo sapiens SH3 protein
interacting with Nck, 90 kDa (AF3P21), transcript”, “variant 1, mRNA”,
gi|37577149|ref|NM_016453.2|[37577149]; 1818: NM_016508, “Homo sapiens cyclin-
dependent kinase-like 3 (CDKL3), mRNA”, gi|17017984|ref|NM_016508.2|[17017984]; 1819:
NM_016526, “Homo sapiens blocked early in transport 1 homolog (S. cerevisiae) like
(BET1L),”, mRNA, gi|34365798|ref|NM_016526.3|[34365798]; 1820: NM_016527, “Homo
sapiens hydroxyacid oxidase 2 (long chain) (HAO2), mRNA”,
gi|7705392|ref|NM_016527.1|[7705392]; 1821: NM_016530, “Homo sapiens RAB8B, member
RAS oncogene family (RAB8B), mRNA”, gi|7706562|ref|NM_016530.1|[7706562]; 1822:
NM_016539, Homo sapiens sirtuin (silent mating type information regulation 2 homolog) 6 (S.,
“cerevisiae) (SIRT6), mRNA”, gi|7706709|ref|NM_016539.1|[7706709]; 1823: NM_016545,
“Homo sapiens immediate early response 5 (IER5), mRNA”,
gi|16554598|ref|NM_016545.2|[16554598]; 1824: NM_016551, “Homo sapiens transmembrane
7 superfamily member 3 (TM7SF3), mRNA”, gi|7706574|ref|NM_016551.1|[7706574]; 1825:
NM_016557, “Homo sapiens chemokine (C—C motif) receptor-like 1 (CCRL1), transcript
variant”, “2, mRNA”, gi|30795218|ref|NM_016557.2|[30795218]; 1826: NM_016558, “Homo
sapiens SCAN domain containing 1 (SCAND1), transcript variant 1, mRNA”,
gi|15967154|ref|NM_016558.2|[15967154]; 1827: NM_016559 “Homo sapiens Pex5p-related
protein (PEX5R), mRNA”, gi|7706670|ref|NM_016559.1|[7706670]; 1828: NM_016561,
“Homo sapiens bifunctional apoptosis regulator (BFAR), mRNA”,
gi|7706090|ref|NM_016561.1|[7706090]; 1829: NM_016567, “Homo sapiens BRCA2 and
CDKN1A interacting protein (BCCIP), transcript variant A,”, mRNA,
gi|17402869|ref|NM_016567.2|[17402869]; 1830: NM_016570, “Homo sapiens PTX1 protein
(PTX1), mRNA”, gi|7706104|ref|NM_016570.1|[7706104]; 1831: NM_016573, “Homo sapiens
Gem-interacting protein (GMIP), mRNA”, gi|7706106|ref|NM_016573.1|[7706106]; 1832:
NM_016576, “Homo sapiens guanosine monophosphate reductase 2 (GMPR2), mRNA”,
gi|20070275|ref|NM_016576.2|[20070275]; 1833: NM_016581, Homo sapiens likely ortholog
of mouse signaling intermediate in Toll, “pathway-evolutionarily conserved (SITPEC), mRNA”,
gi|20149632|ref|NM_016581.2|[20149632]; 1834: NM_016593, “Homo sapiens cytochrome
P450, family 39, subfamily A, polypeptide 1 (CYP39A1),”, mRNA,
gi|32313586|ref|NM_016593.3|[32313586]; 1835: NM_016602, “Homo sapiens G protein-
coupled receptor 2 (GPR2), mRNA”, gi|7705315|ref|NM_016602.1|[7705315]; 1836:
NM_016611, “Homo sapiens potassium channel, subfamily K, member 4 (KCNK4), transcript”,
“variant 1, mRNA”, gi|15718764|ref|NM_016611.2|[15718764]; 1837: NM_016614, “Homo
sapiens TRAF and TNF receptor associated protein (TTRAP), mRNA”,
gi|23510347|ref|NM_016614.2|[23510347]; 1838: NM_016617, “Homo sapiens hypothetical
protein BM-002 (BM-002), mRNA”, gi|7705299|ref|NM_016617.1|[7705299]; 1839:
NM_016621, “Homo sapiens BRAF35/HDAC2 complex (80 kDa) (BHC80), mRNA”,
gi|19923461|ref|NM_016621.2|[19923461]; 1840: NM_016625, “Homo sapiens BM-011 protein
(MGC12197), mRNA”, gi|38488726|ref|NM_016625.2|[38488726]; 1841: NM_016627, “Homo
sapiens hypothetical protein LOC51321 (LOC51321), mRNA”,
gi|42476207|ref|NM_016627.2|[42476207]; 1842: NM_016630, “Homo sapiens acid cluster
protein 33 (ACP33), mRNA”, gi|42544234|ref|NM_016630.3|[42544234]; 1843: NM_016639,
“Homo sapiens tumor necrosis factor receptor superfamily, member 12A (TNFRSF12A),”,
mRNA, gi|7706185|ref|NM_016639.1|[7706185]; 1844: NM_016647, “Homo sapiens
mesenchymal stem cell protein DSCD75 (LOC51337), mRNA”,
gi|7706199|ref|NM_016647.1|[7706199]; 1845: NM_016651, “Homo sapiens dapper homolog
1, antagonist of beta-catenin (xenopus) (DACT1),”, mRNA,
gi|38569506|ref|NM_016651.4|[38569506]; 1846: NM_016831, “Homo sapiens period homolog
3 (Drosophila) (PER3), mRNA”, gi|8567387|ref|NM_01683 1.1|[8567387]; 1847: NM_016937,
“Homo sapiens polymerase (DNA directed), alpha (POLA), mRNA”,
gi|8393994|ref|NM_016937.1|[8393994]; 1848: NM_016940, “Homo sapiens chromosome 21
open reading frame 6 (C21orf6), mRNA”, gi|8393017|ref|NM_016940.1|[8393017]; 1849:
NM_016948, “Homo sapiens par-6 partitioning defective 6 homolog alpha (C. elegans)
(PARD6A),”, mRNA, gi|8394416|ref|NM_016948.1|[8394416]; 1850: NM_017412, “Homo
sapiens frizzled homolog 3 (Drosophila) (FZD3), mRNA”,
gi|22035685|ref|NM_017412.2|[22035685]; 1851: NM_017414, “Homo sapiens ubiquitin
specific protease 18 (USP18), mRNA”, gi|32313609|ref|NM_017414.2|[32313609]; 1852:
NM_017422, “Homo sapiens calmodulin-like 5 (CALML5), mRNA”,
gi|38327636|ref|NM_017422.3|[38327636]; 1853: NM_017426, “Homo sapiens nucleoporin
54 kDa (NUP54), mRNA”, gi|26051236|ref|NM_017426.2|[26051236]; 1854: NM_017429,
“Homo sapiens beta-carotene 15,15′-monooxygenase 1 (BCMO1), mRNA”,
gi|8393364|ref|NM_017429.1|[8393364]; 1855: NM_017435, “Homo sapiens solute carrier
organic anion transporter family, member 1C1”, “(SLCO1C1), mRNA”,
gi|21361594|ref|NM_017435.2|[21361594]; 1856: NM_017443 “Homo sapiens polymerase
(DNA directed), epsilon 3 (p17 subunit) (POLE3), mRNA”,
gi|31543422|ref|NM_017443.3|[31543422]; 1857: NM_017495, “Homo sapiens RNA-binding
region (RNP1, RRM) containing 1 (RNPC1), transcript”, “variant 1, mRNA”,
gi|34577106|ref|NM_17495.4|[34577106]; 1858: NM_017542, “Homo sapiens pogo
transposable element with KRAB domain (POGK), mRNA”,
gi|22027479|ref|NM_017542.3|[22027479]; 1859: NM_017559, “Homo sapiens hypothetical
protein DKFZp434H2215 (DKFZp434H2215), mRNA”,
gi|8922137|ref|NM_017559.1|[8922137]; 1860: NM_017566, “Homo sapiens hypothetical
protein DKFZp434G0522 (DKFZp434G0522), mRNA”,
gi|21314674|ref|NM_017566.2|[21314674]; 1861: NM_017571, “Homo sapiens hypothetical
protein LOC55580 (LOC55580), mRNA”, gi|8923837|ref|NM_017571.1|[8923837]; 1862:
NM_017578, “Homo sapiens ropporin, rhophilin associated protein 1 (ROPN1), mRNA”,
gi|21359919|ref|NM_017578.2|[21359919]; 1863: NM_017582, “Homo sapiens ubiquitin-
conjugating enzyme E2Q (putative) (UBE2Q), mRNA”,
gi|38045949|ref|NM_017582.5|[38045949]; 1864: NM_017589, “Homo sapiens B-cell
translocation gene 4 (BTG4), mRNA”, gi|28872723|ref|NM_017589.2|[28872723]; 1865:
NM_017596,, ref|NM_017596.1|[8922142], This record was temporarily removed by RefSeq
staff for additional review.,, 1866: NM_017606, “Homo sapiens hypothetical protein
DKFZp434K1210 (DKFZp434K1210), mRNA”, gi|40254896|ref|NM_017606.2|[40254896];
1867: NM_017610, “Homo sapiens ring finger protein 111 (RNF111), mRNA”,
gi|37595552|ref|NM_017610.6|[37595552]; 1868: NM_017623, “Homo sapiens cyclin M3
(CNNM3), transcript variant 1, mRNA”, gi|40068048|ref|NM_017623.3|[40068048]; 1869:
NM_017624, “Homo sapiens hypothetical protein FLJ20019 (FLJ20019), mRNA”,
gi|8923025|ref|NM_017624.1|[8923025]; 1870: NM_017629, “Homo sapiens eukaryotic
translation initiation factor 2C, 4 (EIF2C4), mRNA”, gi|29029592|ref|NM_017629.2|[29029592];
1871: NM_017630, “Homo sapiens chromosome 14 open reading frame 113 (C14orf113),
mRNA”, gi|8923035|ref|NM_017630.1|[8923035]; 1872: NM_017631, “Homo sapiens
hypothetical protein FLJ20035 (FLJ20035), mRNA”,
gi|37059778|ref|NM_017631.3|[37059778]; 1873: NM_017632, Homo sapiens
collaborates/cooperates with ARF (alternate reading frame) protein, “(CARF), mRNA”,
gi|8923039|ref|NM_017632.1|[8923039]; 1874: NM_017633, “Homo sapiens chromosome 6
open reading frame 37 (C6orf37), mRNA”, gi|8923041|ref|NM_017633.1|[8923041]; 1875:
NM_017634, “Homo sapiens potassium channel tetramerisation domain containing 9 (KCTD9),
mRNA”, gi|39753958|ref|NM_017634.2|[39753958]; 1876: NM_017636, “Homo sapiens
transient receptor potential cation channel, subfamily M, member 4”, “(TRPM4), mRNA”,
gi|21314670|ref|NM_017636.2|[21314670]; 1877: NM_017645, “Homo sapiens family with
sequence similarity 29, member A (FAM29A), mRNA”,
gi|31377561|ref|NM_017645.3|[31377561]; 1878: NM_017647, “Homo sapiens FtsJ homolog 3
(E. coli) (FTSJ3), mRNA”, gi|17017990|ref|NM_017647.2|[17017990]; 1879: NM_017654,
“Homo sapiens FLJ20073 protein (FLJ20073), mRNA”,
gi|38201705|ref|NM_017654.2|[38201705]; 1880: NM_017655, “Homo sapiens PDZ domain
protein GIPC2 (GIPC2), mRNA”, gi|41393578|ref|NM_017655.4|[41393578]; 1881:
NM_017657, “Homo sapiens hypothetical protein FLJ20080 (FLJ20080), mRNA”,
gi|31377757|ref|NM_017657.2|[31377757]; 1882: NM_017659, “Homo sapiens hypothetical
protein FLJ20084 (FLJ20084), mRNA”, gi|8923091|ref|NM_017659.1|[8923091]; 1883:
NM_017665, “Homo sapiens zinc finger, CCHC domain containing 10 (ZCCHC10), mRNA”,
gi|8923105|ref|NM_017665.1|[8923105]; 1884: NM_017668, “Homo sapiens nudE nuclear
distribution gene E homolog 1 (A. nidulans) (NDE1),”, mRNA,
gi|8923109|ref|NM_017668.1|[8923109]; 1885: NM_017676, “Homo sapiens hypothetical
protein FLJ20125 (FLJ20125), mRNA”, gi|8923123|ref|NM_017676.1|[8923123]; 1886:
NM_017686, “Homo sapiens ganglioside induced differentiation associated protein 2
(GDAP2),”, mRNA, gi|8923142|ref|NM_017686.1|[8923142]; 1887: NM_017691, “Homo
sapiens hypothetical protein FLJ20156 (FLJ20156), mRNA”,
gi|8923153|ref|NM_017691.1|[8923153]; 1888: NM_017699, “Homo sapiens hypothetical
protein FLJ20174 (FLJ20174), mRNA”, gi|8923170|ref|NM_017699.1|[8923170]; 1889:
NM_017702, “Homo sapiens hypothetical protein FLJ20186 (FLJ20186), mRNA”,
gi|8923176|ref|NM_017702.1|[8923176]; 1890: NM_017703, “Homo sapiens F-box and
leucine-rich repeat protein 12 (FBXL12), mRNA”, gi|8923178|ref|NM_017703.1|[8923178];
1891: NM_017704, “Homo sapiens fetal globin-inducing factor (FGIF), mRNA”,
gi|41350197|ref|NM_017704.2|[41350197]; 1892: NM_017708, “Homo sapiens hypothetical
protein FLJ20200 (FLJ20200), mRNA”, gi|8923189|ref|NM_017708.1|[8923189]; 1893:
NM_017709, “Homo sapiens FLJ20202 protein (FLJ20202), mRNA”,
gi|38570094|ref|NM_017709.2|[38570094]; 1894: NM_017720, “Homo sapiens signal-
transducing adaptor protein-2 (STAP2), mRNA”, gi|8923213|ref|NM_017720.1|[8923213];
1895: NM_017727, “Homo sapiens hypothetical protein FLJ20254 (FLJ20254), mRNA”,
gi|19923809|ref|NM_017727.2|[19923809]; 1896: NM_017728, “Homo sapiens hypothetical
protein FLJ20255 (FLJ20255), mRNA”, gi|40254901|ref|NM_017728.2|[40254901]; 1897:
NM_017735, “Homo sapiens hypothetical protein FLJ20272 (FLJ20272), mRNA”,
gi|42476021|ref|NM_017735.3|[42476021]; 1898: NM_017739, “Homo sapiens O-linked
mannose beta1,2-N-acetylglucosaminyltransferase”, “(FLJ20277), mRNA”,
gi|8923252|ref|NM_017739.1|[8923252]; 1899: NM_017740, “Homo sapiens zinc finger,
DHHC domain containing 7 (ZDHHC7), mRNA”, gi|8923254|ref|NM_017740.1|[8923254];
1900: NM_017742, “Homo sapiens zinc finger, CCHC domain containing 2 (ZCCHC2),
mRNA”, gi|41872708|ref|NM_017742.3|[41872708]; 1901: NM_017743, “Homo sapiens
dipeptidylpeptidase 8 (DPP8), transcript variant 2, mRNA”,
gi|37577092|ref|NM_017743.3|[37577092]; 1902: NM_017744, “Homo sapiens suppression of
tumorigenicity 7 like (ST7L), transcript variant 1,”, mRNA,
gi|38201633|ref|NM_017744.4|[38201633]; 1903: NM_017745, “Homo sapiens BCL6 co-
repressor (BCOR), transcript variant 1, mRNA”, gi|21071036|ref|NM_017745.4|[21071036];
1904: NM_017746, “Homo sapiens testis expressed gene 10 (TEX10), mRNA”,
gi|8923268|ref|NM_017746.1|[8923268]; 1905: NM_017748, “Homo sapiens hypothetical
protein FLJ20291 (FLJ20291), mRNA”, gi|34147582|ref|NM_017748.3|[34147582]; 1906:
NM_017750, “Homo sapiens hypothetical protein FLJ20296 (FLJ20296), mRNA”,
gi|31377747|ref|NM_017750.2|[31377747]; 1907: NM_017751, “Homo sapiens hypothetical
protein FLJ20297 (FLJ20297), mRNA”, gi|8923276|ref|NM_017751.1|[8923276]; 1908:
NM_017761, “Homo sapiens proline-rich nuclear receptor coactivator 2 (PNRC2), mRNA”,
gi|20127576|ref|NM_017761.2|[20127576]; 1909: NM_017762, “Homo sapiens hypothetical
protein FLJ20313 (FLJ20313), mRNA”, gi|8923296|ref|NM_017762.1|[8923296]; 1910:
NM_017766 “Homo sapiens hypothetical protein FLJ20321 (FLJ20321), mRNA”,
gi|40254903|ref|NM_017766.2|[40254903]; 1911: NM_017774, “Homo sapiens CDK5
regulatory subunit associated protein 1-like 1 (CDKAL1), mRNA”,
gi|8923317|ref|NM_017774.1|[8923317]; 1912: NM_017791, “Homo sapiens chromosome 14
open reading frame 58 (C14orf58), mRNA”, gi|8923349|ref|NM_017791.1|[8923349]; 1913:
NM_017801, “Homo sapiens chemokine-like factor super family 6 (CKLFSF6), mRNA”,
gi|32130534|ref|NM_017801.2|[32130534]; 1914: NM_017805, “Homo sapiens Ras-interacting
protein (RAIN), mRNA”, gi|38570104|ref|NM_017805.2|[38570104]; 1915: NM_017806,
“Homo sapiens hypothetical protein FLJ20406 (FLJ20406), mRNA”,
gi|8923377|ref|NM_017806.1|[8923377]; 1916: NM_017807, “Homo sapiens O-
sialoglycoprotein endopeptidase (OSGEP), mRNA”, gi|8923379|ref|NM_017807.1|[8923379];
1917: NM_017813, “Homo sapiens hypothetical protein FLJ20421 (FLJ20421), mRNA”,
gi|8923391|ref|NM_017813.1|[8923391]; 1918: NM_017818, “Homo sapiens WD repeat
domain 8 (WDR8), mRNA”, gi|16445433|ref|NM_017818.2|[16445433]; 1919: NM_017827,
“Homo sapiens seryl-tRNA synthetase 2 (SARS2), mRNA”,
gi|20149644|ref|NM_017827.2|[20149644]; 1920: NM_017832, “Homo sapiens hypothetical
protein FLJ20457 (FLJ20457), mRNA”, gi|20127565|ref|NM_017832.2|[20127565]; 1921:
NM_017833, “Homo sapiens chromosome 21 open reading frame 55 (C21orf55), mRNA”,
gi|40254907|ref|NM_017833.2|[40254907]; 1922: NM_017835, “Homo sapiens chromosome 21
open reading frame 59 (C21orf59), mRNA”, gi|8923436|ref|NM_017835.1|[8923436]; 1923:
NM_017837, “Homo sapiens hypothetical protein FLJ20477 (FLJ20477), mRNA”,
gi|21361770|ref|NM_017837.2|[21361770]; 1924: NM_017840, “Homo sapiens mitochondrial
ribosomal protein L16 (MRPL16), nuclear gene encoding”, “mitochondrial protein, mRNA”,
gi|27436902|ref|NM_017840.2|[27436902]; 1925: NM_017847, “Homo sapiens chromosome 1
open reading frame 27 (C1orf27), mRNA”, gi|20127566|ref|NM_017847.2|[20127566]; 1926:
NM_017849, “Homo sapiens hypothetical protein FLJ20507 (FLJ20507), mRNA”,
gi|8923465|ref|NM_017849.1|[8923465]; 1927: NM_017850, “Homo sapiens hypothetical
protein FLJ20508 (FLJ20508), mRNA”, gi|8923468|ref|NM_017850.1|[8923468]; 1928:
NM_017851, “Homo sapiens hypothetical protein FLJ20509 (FLJ20509), mRNA”,
gi|8923470|ref|NM_017851.1|[8923470]; 1929: NM_017856, “Homo sapiens hypothetical
protein FLJ20514 (FLJ20514), mRNA”, gi|8923480|ref|NM_017856.1|[8923480]; 1930:
NM_017859, “Homo sapiens uridine kinase-like 1 (URKL1), mRNA”,
gi|8923486|ref|NM_017859.1|[8923486]; 1931: NM_017860, “Homo sapiens hypothetical
protein FLJ20519 (FLJ20519), mRNA”, gi|34147608|ref|NM_017860.3|[34147608]; 1932:
NM_017867, “Homo sapiens hypothetical protein FLJ20534 (FLJ20534), mRNA”,
gi|8923502|ref|NM_017867.1|[8923502]; 1933: NM_017869, “Homo sapiens BTG3 associated
nuclear protein (BANP), transcript variant 1, mRNA”,
gi|17986265|ref|NM_017869.2|[17986265]; 1934: NM_017870, “Homo sapiens heat shock
70 kDa protein 5 (glucose-regulated protein, 78 kDa)”, “binding protein 1 (HSPA5BP1), transcript
variant 1, mRNA”, gi|30089934|ref|NM_017870.2|[30089934]; 1935: NM_017872, “Homo
sapiens hypothetical protein FLJ20546 (FLJ20546), mRNA”,
gi|21361662|ref|NM_017872.2|[21361662]; 1936: NM_017877, “Homo sapiens chromosome 2
open reading frame 18 (C2orf18), mRNA”, gi|31542710|ref|NM_017877.2|[31542710]; 1937:
NM_017880, “Homo sapiens hypothetical protein FLJ20558 (FLJ20558), mRNA”,
gi|8923527|ref|NM_017880.1|[8923527]; 1938: NM_017890, “Homo sapiens Cohen syndrome
1 (COH1), transcript variant 5, mRNA”, gi|35493712|ref|NM_017890.3|[35493712]; 1939:
NM_017896, “Homo sapiens chromosome 20 open reading frame 11 (C20orf11), mRNA”,
gi|40804466|ref|NM_017896.2|[40804466]; 1940: NM_017901, “Homo sapiens two pore
segment channel 1 (TPCN1), mRNA”, gi|29725621|ref|NM_017901.3|[29725621]; 1941:
NM_017903, “Homo sapiens hypothetical protein FLJ20618 (FLJ20618), mRNA”,
gi|31542714|ref|NM_017903.2|[31542714]; 1942: NM_017908, “Homo sapiens hypothetical
protein FLJ20626 (FLJ20626), mRNA”, gi|8923581|ref|NM_017908.1|[8923581]; 1943;
NM_017909, “Homo sapiens chromosome 6 open reading frame 96 (C6orf96), mRNA”,
gi|8923583|ref|NM_017909.1|[8923583]; 1944: NM_017910, “Homo sapiens hypothetical
protein FLJ20628 (FLJ20628), mRNA”, gi|13435382|ref|NM_017910.2|[13435382]; 1945:
NM_017911, “Homo sapiens hypothetical protein FLJ20635 (FLJ20635), mRNA”,
gi|8923587|ref|NM_017911.1|[8923587]; 1946: NM_017913, “Homo sapiens Hsp90-associating
relative of Cdc37 (HARC), mRNA”, gi|8923591|ref|NM_017913.1|[8923591]; 1947:
NM_017914, “Homo sapiens hypothetical protein FLJ20640 (FLJ20640), mRNA”,
gi|42476017|ref|NM_017914.2|[42476017]; 1948: NM_017915, “Homo sapiens hypothetical
protein FLJ20641 (FLJ20641), mRNA”, gi|8923595|ref|NM_017915.1|[8923595]; 1949:
NM_017916, “Homo sapiens hypothetical protein FLJ20643 (FLJ20643), mRNA”,
gi|8923597|ref|NM_017916.1|[8923597]; 1950: NM_017917, “Homo sapiens chromosome 14
open reading frame 10 (C14orf10), mRNA”, gi|31542241|ref|NM_017917.2|[31542241]; 1951:
NM_017926, “Homo sapiens chromosome 14 open reading frame 118 (C14orf118), transcript”,
“variant 1, mRNA”, gi|40018645|ref|NM_017926.2|[40018645]; 1952: NM_017929, “Homo
sapiens peroxisome biogenesis factor 26 (PEX26), mRNA”,
gi|32307138|ref|NM_017929.2|[32307138]; 1953: NM_017932, “Homo sapiens hypothetical
protein FLJ20700 (FLJ20700), mRNA”, gi|8923629|ref|NM_017932.1|[8923629]; 1954:
NM_017933, “Homo sapiens hypothetical protein FLJ20701 (FLJ20701), mRNA”,
gi|40255259|ref|NM_017933.3|[40255259]; 1955: NM_017937, “Homo sapiens hypothetical
protein FLJ20712 (FL120712), mRNA”, gi|8923640|ref|NM_017937.1|[8923640]; 1956:
NM_017938, “Homo sapiens hypothetical protein FLJ20716 (FLJ20716), mRNA”,
gi|31377736|ref|NM_017938.2|[31377736]; 1957: NM_017941, “Homo sapiens lung cancer-
related protein 8 (HLC-8), mRNA”, gi|34222156|ref|NM_017941.3|[34222156]; 1958:
NM_017945, “Homo sapiens solute carrier family 35, member A5 (SLC35A5), mRNA”,
gi|31543636|ref|NM_017945.2|[31543636]; 1959: NM_017946, “Homo sapiens FK506 binding
protein 14, 22 kDa (FKBP14), mRNA”, gi|8923658|ref|NM_017946.1|[8923658]; 1960:
NM_017952, “Homo sapiens FLJ20758 protein (FLJ20758), mRNA”,
gi|38683854|ref|NM_017952.4|[38683854]; 1961: NM_017964, “Homo sapiens solute carrier
family 30 (zinc transporter), member 6 (SLC30A6),”, mRNA,
gi|21687223|ref|NM_017964.2|[21687223]; 1962: NM_017974, “Homo sapiens APG16
autophagy 16-like (S. cerevisiae) (APG16L), transcript”, “variant 2, mRNA”,
gi|38683867|ref|NM_017974.2|[38683867]; 1963: NM_017975, “Homo sapiens hypothetical
protein FLJ10036 (FLJ10036), mRNA”, gi|33300636|ref|NM_017975.2|[33300636]; 1964:
NM_017979, “Homo sapiens smooth muscle cell associated protein-1 (SMAP-1), mRNA”,
gi|8922201|ref|NM_017979.1|[8922201]; 1965: NM_017985, “Homo sapiens chromosome 9
open reading frame 68 (C9orf68), mRNA”, gi|42538977|ref|NM_017985.2|[42538977]; 1966:
NM_017990, “Homo sapiens pyruvate dehydrogenase phosphatase regulatory subunit (PDPR),
mRNA”, gi|32261325|ref|NM_017990.3|[32261325]; 1967: NM_017991, “Homo sapiens
hypothetical protein FLJ10081 (FLJ10081), mRNA”,
gi|21361733|ref|NM_017991.3|[21361733]; 1968: NM_017999, “Homo sapiens ring finger
protein 31 (RNF31), mRNA”, gi|38045939|ref|NM_017999.3|[38045939]; 1969: NM_018000,
“Homo sapiens hypothetical protein FLJ10116 (FLJ10116), mRNA”,
gi|8922236|ref|NM_018000.1|[8922236]; 1970: NM_018004, “Homo sapiens hypothetical
protein FLJ10134 (FLJ10134), mRNA”, gi|8922242|ref|NM_018004.1|[8922242]; 1971:
NM_018006, “Homo sapiens hypothetical protein FLJ10140 (FLJ10140), mRNA”,
gi|31542640|ref|NM_018006.2|[31542640]; 1972: NM_018011, “Homo sapiens hypothetical
protein FLJ10154 (FLJ10154), mRNA”, gi|8922258|ref|NM_018011.1|[8922258]; 1973:
NM_018022, “Homo sapiens hypothetical protein FLJ10199 (FLJ10199), mRNA”,
gi|8922276|ref|NM_018022.1|[8922276]; 1974: NM_018023, “Homo sapiens hypothetical
protein FLJ10201 (FLJ10201), mRNA”, gi|33620754|ref|NM_018023.3|[33620754]; 1975:
NM_018024, “Homo sapiens hypothetical protein FLJ10204 (FLJ10204), mRNA”,
gi|8922280|ref|NM_018024.1|[8922280]; 1976: NM_018026, “Homo sapiens phosphofurin
acidic cluster sorting protein 1 (PACS1), mRNA”, gi|30089915|ref|NM_018026.2|[30089915];
1977: NM_018034, “Homo sapiens hypothetical protein FLJ10233 (FLJ10233), mRNA”,
gi|20149650|ref|NM_018034.2|[20149650]; 1978: NM_018037, “Homo sapiens Ral-A exchange
factor RalGPS2 (FLJ10244), mRNA”, gi|8922306|ref|NM_018037.1|[8922306]; 1979:
NM_018039, “Homo sapiens jumonji domain containing 2D (JMJD2D), mRNA”,
gi|39653316|ref|NM_018039.2|[39653316]; 1980: NM_018040, “Homo sapiens hypothetical
protein FLJ10252 (FLJ10252), mRNA”, gi|8922312|ref|NM_018040.1|[8922312]; 1981:
NM_018048, “Homo sapiens hypothetical protein FLJ10292 (FLJ10292), mRNA”,
gi|21361685|ref|NM_018048.2|[21361685]; 1982: NM_018049, “Homo sapiens pleckstrin
homology domain containing, family J member 1 (PLEKHJ1),”, mRNA,
gi|8922332|ref|NM_018049.1|[8922332]; 1983: NM_018050, “Homo sapiens hypothetical
protein FLJ10298 (FLJ10298), mRNA”, gi|31542649|ref|NM_018050.2|[31542649]; 1984:
NM_018056, “Homo sapiens hypothetical protein FLJ10315 (FLJ10315), mRNA”,
gi|8922347|ref|NM_018056.1|[8922347]; 1985: NM_018058, “Homo sapiens cartilage acidic
protein 1 (CRTAC1), mRNA”, gi|42415498|ref|NM_018058.2|[42415498]; 1986: NM_018060,
“Homo sapiens mitochondrial isoleucine tRNA synthetase (FLJ10326), mRNA”,
gi|39752644|ref|NM_018060.2|[39752644]; 1987: NM_018061, “Homo sapiens hypothetical
protein FLJ10330 (FLJ10330), mRNA”, gi|8922357|ref|NM_018061.1|[8922357]; 1988:
NM_018064, “Homo sapiens chromosome 6 open reading frame 166 (C6orf166), mRNA”,
gi|39725640|ref|NM_018064.2|[39725640]; 1989: NM_018066, “Homo sapiens hypothetical
protein FLJ10349 (FLJ10349), mRNA”, gi|40254894|ref|NM_018066.2|[40254894]; 1990:
NM_018074, “Homo sapiens hypothetical protein FLJ10374 (FLJ10374), mRNA”,
gi|34222335|ref|NM_018074.3|[34222335]; 1991: NM_018077, “Homo sapiens hypothetical
protein FLJ10377 (FLJ10377), mRNA”, gi|8922387|ref|NM_018077.1|[8922387]; 1992:
NM_018079, “Homo sapiens hypothetical protein FLJ10379 (FLJ10379), mRNA”,
gi|39841072|ref|NM_018079.3|[39841072]; 1993: NM_018083, “Homo sapiens zinc finger
protein 358 (ZNF358), mRNA”, gi|8922400|ref|NM_018083.1|[8922400]; 1994: NM_018090,
“Homo sapiens hypothetical protein FLJ10420 (FLJ10420), mRNA”,
gi|39725692|ref|NM_018090.3|[39725692]; 1995: NM_018091, “Homo sapiens elongation
protein 3 homolog (S. cerevisiae) (ELP3), mRNA”, gi|23510282|ref|NM_018091.3|[23510282];
1996: NM_018097, “Homo sapiens hypothetical protein FLJ10460 (FLJ10460), mRNA”,
gi|8922429|ref|NM_018097.1|[8922429]; 1997: NM_018101, “Homo sapiens cell division cycle
associated 8 (CDCA8), mRNA”, gi|8922437|ref|NM_018101.1|[8922437]; 1998: NM_018105,
“Homo sapiens THAP domain containing, apoptosis associated protein 1 (THAP1),”, “transcript
variant 1, mRNA”, gi|40068498|ref|NM_018105.2|[40068498]; 1999: NM_018106, “Homo
sapiens zinc finger, DHHC domain containing 4 (ZDHHC4), mRNA”,
gi|21361700|ref|NM_018106.2|[21361700]; 2000: NM_018107, “Homo sapiens RNA-binding
region (RNP1, RRM) containing 4 (RNPC4), mRNA”,
gi|34147682|ref|NM_018107.3|[34147682]; 2001: NM_018108, “Homo sapiens chromosome 14
open reading frame 130 (C14orf130), mRNA”, gi|21361696|ref|NM_018108.2|[21361696];
2002: NM_018111, “Homo sapiens hypothetical protein FLJ10490 (FLJ10490), mRNA”,
gi|8922458|ref|NM_018111.1|[8922458]; 2003: NM_018116, “Homo sapiens misato
(FLJ10504), mRNA”, gi|39780570|ref|NM_018116.2|[39780570]; 2004: NM_018117, “Homo
sapiens WD repeat domain 11 (WDR11), mRNA”, gi|22547233|ref|NM_018117.10|[22547233];
2005: NM_018118, Homo sapiens MCM3 minichromosome maintenance deficient 3 (S. cerevisiae),
“associated protein, antisense (MCM3APAS), mRNA”,
gi|8922473|ref|NM_018118.1|[8922473]; 2006: NM_018119, “Homo sapiens RNA polymerase
III 80 kDa subunit RPC5 (RPC5), mRNA”, gi|38146100|ref|NM_018119.2|[38146100]; 2007:
NM_018124, “Homo sapiens hypothetical protein FLJ10520 (FLJ10520), mRNA”,
gi|19923516|ref|NM_018124.2|[19923516]; 2008: NM_018126, “Homo sapiens hypothetical
protein FLJ10525 (FLJ10525), mRNA”, gi|8922490|ref|NM_018126.1|[8922490]; 2009:
NM_018131, “Homo sapiens chromosome 10 open reading frame 3 (C10orf3), mRNA”,
gi|34147683|ref|NM_018131.3|[34147683]; 2010: NM_018132, “Homo sapiens chromosome 6
open reading frame 139 (C6orf139), mRNA”, gi|40068060|ref|NM_018132.2|[40068060]; 2011:
NM_018133, “Homo sapiens hypothetical protein FLJ10546 (FLJ10546), mRNA”,
gi|38570120|ref|NM_018133.2|[38570120]; 2012: NM_018139, “Homo sapiens chromosome 14
open reading frame 104 (C14orf104), mRNA”, gi|8922518|ref|NM_018139.1|[8922518]; 2013:
NM_018141, “Homo sapiens mitochondrial ribosomal protein S10 (MRPS10), nuclear gene
encoding”, “mitochondrial protein, mRNA”, gi|16554606|ref|NM_018141.2|[16554606]; 2014:
NM_018143, “Homo sapiens kelch-like 11 (Drosophila) (KLHL11), mRNA”,
gi|8922527|ref|NM_018143.1|[8922527]; 2015: NM_018145, “Homo sapiens hypothetical
protein FLJ10579 (FLJ10579), mRNA”, gi|8922531|ref|NM_018145.1|[8922531]; 2016:
NM_018154, “Homo sapiens ASF1 anti-silencing function 1 homolog B (S. cerevisiae)
(ASF1B),”, mRNA, gi|8922548|ref|NM_018154.1|[8922548]; 2017: NM_018158, “Homo
sapiens solute carrier family 4 (anion exchanger), member 1, adaptor”, “protein (SLC4A1AP),
mRNA”, gi|8922556|ref|NM_018158.1|[8922556]; 2018: NM_018163, “Homo sapiens
hypothetical protein FLJ10634 (FLJ10634), mRNA”, gi|8922562|ref|NM_018163.1|[8922562];
2019: NM_018164, “Homo sapiens hypothetical protein FLJ10637 (FLJ10637), mRNA”,
gi|11024685|ref|NM_018164.1|[11024685]; 2020: NM_018171, “Homo sapiens DIP13 beta
(DIP13B), mRNA”, gi|24586662|ref|NM_018171.2|[24586662]; 2021: NM_018172, “Homo
sapiens hypothetical protein FLJ10661 (FLJ10661), mRNA”,
gi|8922578|ref|NM_018172.1|[8922578]; 2022: NM_018174, “Homo sapiens VCY2 interacting
protein 1 (VCY2IP1), mRNA”, gi|21361667|ref|NM_018174.3|[21361667]; 2023: NM_018178,
“Homo sapiens GPP34-related protein (GPP34R), mRNA”,
gi|29826327|ref|NM_018178.3|[29826327]; 2024: NM_018179, “Homo sapiens activating
transcription factor 7 interacting protein (ATF7IP),”, mRNA,
gi|38261961|ref|NM_018179.3|[38261961]; 2025: NM_018181, “Homo sapiens zinc finger
protein 532 (ZNF532), mRNA”, gi|24475845|ref|NM_018181.3|[24475845]; 2026: NM_018182,
“Homo sapiens hypothetical protein FLJ10700 (FLJ10700), mRNA”,
gi|8922595|ref|NM_018182.1|[8922595]; 2027: NM_018191, Homo sapiens regulator of
chromosome condensation (RCC1) and BTB (POZ) domain, “containing protein 1 (RCBTB1),
mRNA”, gi|19923518|ref|NM_018191.2|[19923518]; 2028: NM_018195, “Homo sapiens
hypothetical protein FLJ10726 (FLJ10726), mRNA”,
gi|40254918|ref|NM_018195.2|[40254918]; 2029: NM_018200, “Homo sapiens high-mobility
group 20A (HMG20A), mRNA”, gi|21359925|ref|NM_018200.2|[21359925]; 2030: NM_018202,
“Homo sapiens hypothetical protein FLJ10747 (FLJ10747), mRNA”,
gi|31542660|ref|NM_018202.2|[31542660]; 2031: NM_018205, “Homo sapiens hypothetical
protein FLJ10751 (FLJ10751), mRNA”, gi|8922643|ref|NM_018205.1|[8922643]; 2032:
NM_018206, “Homo sapiens vacuolar protein sorting 35 (yeast) (VPS35), mRNA”,
gi|41352714|ref|NM_018206.3|[41352714]; 2033: NM_018216, “Homo sapiens pantothenate
kinase 4 (PANK4), mRNA”, gi|8922664|ref|NM_018216.1|[8922664]; 2034: NM_018217,
“Homo sapiens chromosome 20 open reading frame 31 (C20orf31), mRNA”,
gi|8922666|ref|NM_018217.1|[8922666]; 2035: NM_018223, “Homo sapiens checkpoint with
forkhead and ring finger domains (CHFR), mRNA”, gi|8922674|ref|NM_018223.1|[8922674];
2036: NM_018225, “Homo sapiens homolog of C. elegans smu-1 (SMU-1), mRNA”,
gi|8922678|ref|NM_018225.1|[8922678]; 2037: NM_018226, “Homo sapiens arginyl
aminopeptidase (aminopeptidase B)-like 1 (RNPEPL1), mRNA”,
gi|20070295|ref|NM_018226.2|[20070295]; 2038: NM_018227, “Homo sapiens hypothetical
protein FLJ10808 (FLJ10808), mRNA”, gi|40255038|ref|NM_018227.3|[40255038]; 2039:
NM_018233, “Homo sapiens hypothetical protein FLJ10826 (FLJ10826), mRNA”,
gi|42476029|ref|NM_018233.2|[42476029]; 2040: NM_018241, “Homo sapiens hypothetical
protein FLJ10846 (FLJ10846), mRNA”, gi|8922706|ref|NM_018241.1|[8922706]; 2041:
NM_018245, “Homo sapiens hypothetical protein FLJ10851 (FLJ10851), mRNA”,
gi|8922715|ref|NM_018245.1|[8922715]; 2042: NM_018246, “Homo sapiens hypothetical
protein FLJ10853 (FLJ10853), mRNA”, gi|8922717|ref|NM_018246.1|[8922717]; 2043:
NM_018247, “Homo sapiens chromosome 6 open reading frame 67 (C6orf67), mRNA”,
gi|8922719|ref|NM_018247.1|[8922719]; 2044: NM_018248, “Homo sapiens DNA glycosylase
hFPG2 (FLJ10858), mRNA”, gi|8922721|ref|NM_018248.1|[8922721]; 2045: NM_018250,
“Homo sapiens hypothetical protein FLJ10871 (FLJ10871), mRNA”,
gi|8922725|ref|NM_018250.1|[8922725]; 2046: NM_018254, “Homo sapiens hypothetical
protein FLJ10876 (FLJ10876), mRNA”, gi|33620752|ref|NM_018254.2|[33620752]; 2047:
NM_018256, “Homo sapiens WD repeat domain 12 (WDR12), mRNA”,
gi|16445423|ref|NM_018256.2|[16445423]; 2048: NM_018259, “Homo sapiens
tetratricopeptide repeat domain 17 (TTC17), mRNA”,
gi|41055004|ref|NM_018259.3|[41055004]; 2049: NM_018261, “Homo sapiens SEC3-like 1 (S. cerevisiae)
(SEC3L1), transcript variant 1, mRNA”, gi|30410719|ref|NM_018261.2|[30410719];
2050: NM_018263, “Homo sapiens additional sex combs like 2 (Drosophila) (ASXL2),
mRNA”, gi|38146000|ref|NM_018263.2|[38146000]; 2051: NM_018264, “Homo sapiens
hypothetical protein FLJ10900 (FLY10900), mRNA”, gi|8922751|ref|NM_018264.1|[8922751];
2052: NM_018265, “Homo sapiens hypothetical protein FLJ10901 (FLJ10901), mRNA”,
gi|8922753|ref|NM_018265.1|[8922753]; 2053: NM_018266, ”Homo sapiens hypothetical
protein FLJ10902 (FLJ10902), mRNA”, gi|8922755|ref|NM_018266.1|[8922755]; 2054:
NM_018270, “Homo sapiens chromosome 20 open reading frame 20 (C20orf20), mRNA”,
gi|40353206|ref|NM_018270.3|[40353206]; 2055: NM_018273, “Homo sapiens hypothetical
protein FLJ10922 (FLJ10922), mRNA”, gi|32171253|ref|NM_018273.2|[32171253]; 2056:
NM_018279, “Homo sapiens hypothetical protein FLJ10936 (FLJ10936), mRNA”,
gi|21361719|ref|NM_018279.2|[21361719]; 2057: NM_018281, “Homo sapiens hypothetical
protein FLJ10948 (FLJ10948), mRNA”, gi|8922786|ref|NM_018281.1|[8922786]; 2058:
NM_018287 “Homo sapiens Rho GTPase activating protein 12 (ARHGAP12), mRNA”,
gi|26986533|ref|NM_018287.4|[26986533]; 2059: NM_018295, “Homo sapiens hypothetical
protein FLJ11000 (FLJ11000), mRNA”, gi|8922813|ref|NM_018295.1|[8922813]; 2060:
NM_018303, “Homo sapiens SEC5-like 1 (S. cerevisiae) (SEC5L1), mRNA”,
gi|30581133|ref|NM_018303.4|[30581133]; 2061: NM_018308, “Homo sapiens acyl-Coenzyme
A oxidase-like (ACOXL), mRNA”, gi|8922839|ref|NM_018308.1|[8922839]; 2062: NM_018314,
“Homo sapiens ubiquitin-conjugating enzyme E2-like (UEV3), mRNA”,
gi|23943813|ref|NM_018314.2|[23943813]; 2063: NM_018317, “Homo sapiens hypothetical
protein FLJ11082 (FLJ11082), mRNA”, gi|8922855|ref|NM_018317.1|[8922855]; 2064:
NM_018319, “Homo sapiens tyrosyl-DNA phosphodiesterase 1 (TDP1), mRNA”,
gi|20127585|ref|NM_018319.2|[20127585]; 2065: NM_018320, “Homo sapiens ring finger
protein 121 (RNF121), transcript variant 1, mRNA”, gi|37588863|ref|NM_018320.3|[37588863];
2066: NM_018327, “Homo sapiens chromosome 20 open reading frame 38 (C20orf38),
mRNA”; gi|8922874|ref|NM_018327.1|[8922874]; 2067: NM_018329, “Homo sapiens
hypothetical protein FLJ11117 (FLJ11117), mRNA”, gi|8922878|ref|NM_018329.1|[8922878];
2068: NM_018338, “Homo sapiens hypothetical protein FLJ11142 (FLJ11142), mRNA”,
gi|31377845|ref|NM_018338.2|[31377845]; 2069: NM_018350,, ref|NM_018350.1|[8922918],
This record was temporarily removed by RefSeq staff for additional review.,, 2070:
NM_018353, “Homo sapiens chromosome 14 open reading frame 106 (C14orf106), mRNA”,
gi|42415491|ref|NM_018353.3|[42415491]; 2071: NM_018354, “Homo sapiens chromosome 20
open reading frame 46 (C20orf46), mRNA”, gi|8922926|ref|NM_018354.1|[8922926]; 2072:
NM_018356, “Homo sapiens hypothetical protein FLJ11193 (FLJ11193), mRNA”,
gi|8922930|ref|NM_018356.1|[8922930]; 2073: NM_018357, “Homo sapiens acheron
(FLJ11196), transcript variant 1, mRNA”, gi|37537709|ref|NM_018357.2|[37537709]; 2074:
NM_018360, “Homo sapiens chromosome X open reading frame 15 (CXorf15), mRNA”,
gi|8922939|ref|NM_018360.1|[8922939]; 2075: NM_018368, “Homo sapiens chromosome 6
open reading frame 209 (C6orf209), mRNA”, gi|31542670|ref|NM_018368.2|[31542670]; 2076:
NM_018372, “Homo sapiens receptor-interacting factor 1 (RIF1), mRNA”,
gi|31377732|ref|NM_018372.2|[31377732]; 2077: NM_018374, “Homo sapiens hypothetical
protein FLJ11273 (FLJ11273), mRNA”, gi|40254892|ref|NM_018374.2|[40254892]; 2078:
NM_018375, “Homo sapiens solute carrier family 39 (zinc transporter), member 9
(SLC39A9),”, mRNA, gi|40254927|ref|NM_018375.2|[40254927]; 2079: NM_018378, “Homo
sapiens F-box and leucine-rich repeat protein 8 (FBXL8), mRNA”,
gi|22547145|ref|NM_018378.2|[22547145]; 2080: NM_018379, “Homo sapiens hypothetical
protein FLJ11280 (FLJ11280), mRNA”, gi|31377840|ref|NM_018379.2|[31377840]; 2081:
NM_018383, “Homo sapiens WD repeat domain 33 (WDR33), mRNA”,
gi|19923528|ref|NM_018383.2|[19923528]; 2082: NM_018386, “Homo sapiens hypothetical
protein FLJ11305 (FLJ11305), mRNA”, gi|8922986|ref|NM_018386.1|[8922986]; 2083:
NM_018388, “Homo sapiens muscleblind-like 3 (Drosophila) (MBNL3), mRNA”,
gi|19387843|ref|NM_018388.2|[19387843]; 2084: NM_018389, “Homo sapiens solute carrier
family 35, member C1 (SLC35C1), mRNA”, gi|37059730|ref|NM_018389.3|[37059730]; 2085:
NM_018398, “Homo sapiens calcium channel, voltage-dependent, alpha 2/delta 3 subunit”,
“(CACNA2D3), mRNA”, gi|8923764|ref|NM_018398.1|[8923764]; 2086: NM_018403, “Homo
sapiens transcription factor SMIF (HSA275986), mRNA”,
gi|8923766|ref|NM_018403.1|[8923766]; 2087: NM_018410, “Homo sapiens hypothetical
protein DKFZp762E1312 (DKFZp762E1312), mRNA”,
gi|21361746|ref|NM_018410.2|[21361746]; 2088: NM_018418, “Homo sapiens
spermatogenesis associated 7 (SPATA7), mRNA”, gi|13384599|ref|NM_018418.1|[13384599];
2089: NM_018419, “Homo sapiens SRY (sex determining region Y)-box 18 (SOX18), mRNA”,
gi|31077201|ref|NM_018419.2|[31077201]; 2090: NM_018422, “Homo sapiens hypothetical
protein DKFZp761K1423 (DKFZp761K1423), mRNA”,
gi|8922171|ref|NM_018422.1|[8922171]; 2091: NM_018428, “Homo sapiens hepatocellular
carcinoma-associated antigen 66 (HCA66), mRNA”, gi|8923721|ref|NM_018428.1|[8923721];
2092: NM_018431, “Homo sapiens docking protein 5 (DOK5), transcript variant 1, mRNA”,
gi|29544725|ref|NM_018431.2|[29544725]; 2093: NM_018433, “Homo sapiens jumonji domain
containing 1 (JMJD1), mRNA”, gi|20357521|ref|NM_018433.2|[20357521]; 2094: NM_018441,
“Homo sapiens peroxisomal trans-2-enoyl-CoA reductase (PECR), mRNA”,
gi|19923816|ref|NM_018441.2|[19923816]; 2095: NM_018444, “Homo sapiens protein
phosphatase 2C, magnesium-dependent, catalytic subunit”, “(PPM2C), mRNA”,
gi|8923959|ref|NM_018444.1|[8923959]; 2096: NM_018452, “Homo sapiens chromosome 6
open reading frame 35 (C6orf35), mRNA”, gi|24431986|ref|NM_018452.2|[24431986]; 2097:
NM_018453, “Homo sapiens chromosome 14 open reading frame 11 (C14orf11), mRNA”,
gi|30425545|ref|NM_018453.2|[30425545]; 2098: NM_018457, “Homo sapiens DKFZp564J157
protein (DKFZP564J157), mRNA”, gi|35250772|ref|NM_018457.2|[35250772]; 2099:
NM_018459,, ref|NM_018459.1|[8922103], This record was replaced or removed. See revision
history for details.,, 2100: NM_018464, “Homo sapiens chromosome 10 open reading frame 70
(C10orf70), mRNA”, gi|8923929|ref|NM_018464.1|[8923929]; 2101: NM_018465, “Homo
sapiens chromosome 9 open reading frame 46 (C9orf46), mRNA”,
gi|8923931|ref|NM_018465.1|[8923931]; 2102: NM_018469, “Homo sapiens uncharacterized
hypothalamus protein HT008 (HT008), mRNA”, gi|38679908|ref|NM_018469.3|[38679908];
2103: NM_018473, “Homo sapiens thioesterase superfamily member 2 (THEM2), mRNA”,
gi|40549423|ref|NM_018473.2|[40549423]; 2104: NM_018474, “Homo sapiens chromosome 20
open reading frame 19 (C20orf19), mRNA”, gi|32189414|ref|NM_018474.2|[32189414]; 2105:
NM_018478, “Homo sapiens chromosome 20 open reading frame 35 (C20orf35), mRNA”,
gi|8923782|ref|NM_018478.1|[8923782]; 2106: NM_018480, “Homo sapiens uncharacterized
hypothalamus protein HT007 (HT007), mRNA”, gi|32189381|ref|NM_018480.2|[32189381];
2107: NM_018484, “Homo sapiens solute carrier family 22 (organic anion/cation transporter),
member”, “11 (SLC22A11), mRNA”, gi|24497483|ref|NM_018484.2|[24497483]; 2108:
NM_018487, “Homo sapiens hepatocellular carcinoma-associated antigen 112 (HCA112),
mRNA”, gi|32484986|ref|NM_018487.2|[32484986]; 2109: NM_018489, “Homo sapiens ash1
(absent, small, or homeotic)-like (Drosophila) (ASH1L), mRNA”,
gi|8922080|ref|NM_018489.1|[8922080]; 2110: NM_018557, Homo sapiens low density
lipoprotein-related protein 1B (deleted in tumors), “(LRP1B), mRNA”,
gi|9055269|ref|NM_018557.1|[9055269]; 2111: NM_018569, “Homo sapiens hypothetical
protein PRO0971 (PRO0971), mRNA”, gi|21361756|ref|NM_018569.2|[21361756]; 2112:
NM_018584, “Homo sapiens calcium/calmodulin-dependent protein kinase II (CaMKIINalpha),
mRNA”, gi|31324542|ref|NM_018584.4|[31324542]; 2113: NM_018589, “Homo sapiens
chromosome 14 open reading frame 116 (C14orf116), mRNA”,
gi|20127573|ref|NM_018589.2|[20127573]; 2114: NM_018590, “Homo sapiens chondroitin
sulfate GalNAcT-2 (GALNACT-2), mRNA”, gi|24429591|ref|NM_018590.3|[24429591]; 2115:
NM_018602, “Homo sapiens DnaJ (Hsp40) homolog, subfamily A, member 4 (DNAJA4),
mRNA”, gi|33354248|ref|NM_018602.2|[33354248]; 2116: NM_018622, “Homo sapiens
presenilin associated, rhomboid-like (PSARL), mRNA”,
gi|20127651|ref|NM_018622.3|[20127651]; 2117: NM_018640, “Homo sapiens neuronal
specific transcription factor DAT1 (DAT1), mRNA”,
gi|41350202|ref|NM_018640.3|[41350202]; 2118: NM_018641, “Homo sapiens carbohydrate
(chondroitin 4) sulfotransferase 12 (CHST12), mRNA”,
gi|20070291|ref|NM_018641.2|[20070291]; 2119: NM_018644, “Homo sapiens beta-1,3-
glucuronyltransferase 1 (glucuronosyltransferase P)”, “(B3GAT1), transcript variant 1, mRNA”,
gi|16905508|ref|NM_018644.2|[16905508]; 2120: NM_018648, “Homo sapiens nucleolar
protein family A, member 3 (H/ACA small nucleolar RNPs)”, “(NOLA3), mRNA”,
gi|15011920|ref|NM_018648.2|[15011920]; 2121: NM_018649, “Homo sapiens H2A histone
family, member Y2 (H2AFY2), mRNA”, gi18923919|ref|NM_018649.1|[8923919]; 2122:
NM_018650, “Homo sapiens MAP/microtubule affinity-regulating kinase 1 (MARK1),
mRNA”, gi|33589842|ref|NM_018650.2|[33589842]; 2123: NM_018654, “Homo sapiens G
protein-coupled receptor, family C, group 5, member D (GPRC5D),”, mRNA,
gi|8923704|ref|NM_018654.1|[8923704]; 2124: NM_018674, “Homo sapiens amiloride-
sensitive cation channel 4, pituitary (ACCN4), transcript”, “variant 1, mRNA”,
gi|33519441|ref|NM_018674.3|[33519441]; 2125: NM_018687, “Homo sapiens hepatocellular
carcinoma-associated gene TD26 (LOC55908), mRNA”,
gi|33667073|ref|NM_018687.3|[33667073]; 2126: NM_018688, “Homo sapiens bridging
integrator 3 (BIN3), mRNA”, gi|39725693|ref|NM_018688.3|[39725693]; 2127: NM_018695,
“Homo sapiens erbb2 interacting protein (ERBB2IP), mRNA”,
gi|8923908|ref|NM_018695.1|[8923908]; 2128: NM_018696, “Homo sapiens elaC homolog 1
(E. coli) (ELAC1), mRNA”, gi|8922121|ref|NM_018696.1|[8922121]; 2129: NM_018697,
Homo sapiens LanC lantibiotic synthetase component C-like 2 (bacterial), “(LANCL2), mRNA”,
gi|19923550|ref|NM_018697.2|[19923550]; 2130: NM_018704, “Homo sapiens hypothetical
protein DKFZp547A023 (DKFZp547A023), mRNA”,
gi|24308178|ref|NM_018704.1|[24308178]; 2131: NM_018705,, ref|NM_018705.1|[8922152],
This record was temporarily removed by RefSeq staff for additional review.,, 2132:
NM_018722, “Homo sapiens BWRT protein (HSA404617), mRNA”,
gi|10190657|ref|NM_018722.1|[10190657]; 2133: NM_018723, “Homo sapiens ataxin 2-
binding protein 1 (A2BP1), transcript variant 4, mRNA”,
gi|22538402|ref|NM_018723.2|[22538402]; 2134: NM_018725, “Homo sapiens interleukin 17
receptor B (IL17RB), transcript variant 1, mRNA”, gi|27477073|ref|NM_018725.2|[27477073];
2135: NM_018845, “Homo sapiens stromal cell protein (LOC55974), mRNA”,
gi|10047123|ref|NM_018845.1|[10047123]; 2136: NM_018897, “Homo sapiens dynein,
axonemal, heavy polypeptide 7 (DNAH7), mRNA”, gi|17864091|ref|NM_018897.1|[17864091];
2137: NM_018943, “Homo sapiens tubulin, alpha 8 (TUBA8), mRNA”,
gi|9507214|ref|NM_018943.1|[9507214]; 2138: NM_018945, “Homo sapiens phosphodiesterase
7B (PDE7B), mRNA”, gi|40255306|ref|NM_018945.2|[40255306]; 2139: NM_018947, “Homo
sapiens cytochrome c, somatic (CYCS), nuclear gene encoding mitochondrial”, “protein,
mRNA”, gi|34328939|ref|NM_018947.4|[34328939]; 2140: NM_018957, “Homo sapiens SH3-
domain binding protein 1 (SH3BP1), mRNA”, gi|15147251|ref|NM_018957.2|[15147251]; 2141:
NM_018959, “Homo sapiens DAZ associated protein 1 (DAZAP1), transcript variant 2,
mRNA”, gi|25470885|ref|NM_018959.2|[25470885]; 2142: NM_018967, “Homo sapiens
syntrophin, gamma 1 (SNTG1), mRNA”, gi|9507162|ref|NM_018967.1|[9507162]; 2143:
NM_018973, “Homo sapiens dolichyl-phosphate mannosyltransferase polypeptide 3 (DPM3),”,
“transcript variant 1, mRNA”, gi|24430133|ref|NM_018973.3|[24430133]; 2144: NM_018975,
“Homo sapiens telomeric repeat binding factor 2, interacting protein (TERF2IP),”, mRNA,
gi|9507032|ref|NM_018975.1|[9507032]; 2145: NM_018982, “Homo sapiens hypothetical
protein DJ167A19.1 (DJ167A19.1), mRNA”, gi|40538797|ref|NM_018982.3|[40538797]; 2146:
NM_018983, “Homo sapiens nucleolar protein family A, member 1 (H/ACA small nucleolar
RNPs)”, “(NOLA1), transcript variant 1, mRNA”, gi|15011914|ref|NM_018983.2|[15011914];
2147: NM_018990, “Homo sapiens chromosome X open reading frame 9 (CXorf9), mRNA”,
gi|40254885|ref|NM_018990.2|[40254885]; 2148: NM_018992, “Homo sapiens potassium
channel tetramerisation domain containing 5 (KCTD5), mRNA”,
gi|9506650|ref|NM_018992.1|[9506650]; 2149: NM_018993, “Homo sapiens Ras and Rab
interactor 2 (RIN2), mRNA”, gi|35493905|ref|NM_018993.2|[35493905]; 2150: NM_019002,
“Homo sapiens ETAA16 protein (ETAA16), mRNA”,
gi|37059813|ref|NM_019002.2|[37059813]; 2151: NM_019006, “Homo sapiens protein
associated with PRK1 (AWP1), mRNA”, gi|21359917|ref|NM_019006.2|[21359917]; 2152:
NM_019008,, ref|NM_019008.4|[42766427]; 2153: NM_019009, “Homo sapiens toll
interacting protein (TOLLIP), mRNA”, gi|21361618|ref|NM_019009.2|[21361618]; 2154:
NM_019014, “Homo sapiens polymerase (RNA) I polypeptide B, 128 kDa (POLR1B), mRNA”,
gi|33469940|ref|NM_019014.2|[33469940]; 2155: NM_019020, “Homo sapiens TBC1 domain
family, member 16 (TBC1D16), mRNA”, gi|33563375|ref|NM_019020.2|[33563375]; 2156:
NM_019021, “Homo sapiens hypothetical protein FLJ20010 (FLJ20010), mRNA”,
gi|9506646|ref|NM_019021.1|[9506646]; 2157: NM_019023, “Homo sapiens hypothetical
protein FLJ10640 (FLJ10640), mRNA”, gi|9506614|ref|NM_019023.1|[9506614]; 2158:
NM_019033, “Homo sapiens hypothetical protein FLJ11235 (FLJ11235), mRNA”,
gi|9506642|ref|NM_019033.1|[9506642]; 2159: NM_019040, “Homo sapiens elongation protein
4 homolog (S. cerevisiae) (ELP4), mRNA”, gi|21361628|ref|NM_019040.2|[21361628]; 2160:
NM_019045, “Homo sapiens similar to rab11-binding protein (DKFZp686L20145), mRNA”,
gi|32526902|ref|NM_019045.2|[32526902]; 2161: NM_019055, “Homo sapiens roundabout
homolog 4, magic roundabout (Drosophila) (ROBO4), mRNA”,
gi|17511434|ref|NM_019055.4|[17511434]; 2162: NM_019056, “Homo sapiens neuronal
protein 17.3 (P17.3), mRNA”, gi|20127560|ref|NM_019056.2|[20127560]; 2163: NM_019059,
Homo sapiens translocase of outer mitochondrial membrane 7 homolog (yeast), “(TOMM7),
mRNA”, gi|9506858|ref|NM_019059.1|[9506858]; 2164: NM_019063, “Homo sapiens
echinoderm microtubule associated protein like 4 (EML4), mRNA”,
gi|19923496|ref|NM_019063.2|[19923496]; 2165: NM_019064, “Homo sapiens sidekick
homolog 2 (chicken) (SDK2), mRNA”, gi|21735576|ref|NM_019064.2|[21735576]; 2166:
NM_019069, “Homo sapiens WD repeat domain 5B (WDR5B), mRNA”,
gi|42544246|ref|NM_019069.3|[42544246]; 2167: NM_019074, “Homo sapiens delta-like 4
(Drosophila) (DLL4), mRNA”, gi|31881762|ref|NM_019074.2|[31881762]; 2168: NM_019081,
“Homo sapiens limkain b1 (LKAP), transcript variant 2, mRNA”,
gi|34878696|ref|NM_019081.2|[34878696]; 2169: NM_019082, “Homo sapiens DEAD (Asp-
Glu-Ala-Asp) box polypeptide 56 (DDX56), mRNA”, gi|9506930|ref|NM_019082.1|[9506930];
2170: NM_019083, “Homo sapiens hypothetical protein FLJ10287 (FLJ10287), mRNA”,
gi|11024703|ref|NM_019083.1|[11024703]; 2171: NM_019088, “Homo sapiens hypothetical
protein F23149_1 (PD2), mRNA”, gi|42476168|ref|NM_019088.2|[42476168]; 2172:
NM_019096, “Homo sapiens GTP binding protein 2 (GTPBP2), mRNA”,
gi|19923498|ref|NM_019096.2|[19923498]; 2173: NM_019102, “Homo sapiens homeo box A5
(HOXA5), mRNA”, gi|24497516|ref|NM_019102.2|[24497516]; 2174: NM_019103, “Homo
sapiens hypothetical protein LOC55954 (LOC55954), mRNA”,
gi|9506862|ref|NM_019103.1|[9506862]; 2175: NM_019104, “Homo sapiens protein F25965
(F25965), mRNA”, gi|28144915|ref|NM_019104.1|[28144915]; 2176: NM_019112, “Homo
sapiens ATP-binding cassette, sub-family A (ABC1), member 7 (ABCA7),”, “transcript variant
1, mRNA”, gi|15451836|ref|NM_019112.2|[15451836]; 2177: NM_019613, “Homo sapiens
hypothetical protein 628 (LOC56270), mRNA”, gi|19923554|ref|NM_019613.2|[19923554];
2178: NM_019843, Homo sapiens eukaryotic translation initiation factor 4E nuclear import
factor 1, “(EIF4ENIF1), mRNA”, gi|10947034|ref|NM_019843.2|[10947034]; 2179:
NM_019845, “Homo sapiens candidate mediator of the p53-dependent G2 arrest (REPRIMO),
mRNA”, gi|9790192|ref|NM_019845.1|[9790192]; 2180: NM_019848, “Homo sapiens solute
carrier family 10 (sodium/bile acid cotransporter family),”, “member 3 (SLC10A3), mRNA”,
gi|10938005|ref|NM_019848.2|[10938005]; 2181: NM_019851, “Homo sapiens fibroblast
growth factor 20 (FGF20), mRNA”, gi|9789946|ref|NM_019851.1|[9789946]; 2182:
NM_019852, “Homo sapiens methyltransferase like 3 (METTL3), mRNA”,
gi|21361826|ref|NM_019852.2|[21361826]; 2183: NM_019857, “Homo sapiens CTP synthase II
(CTPS2), transcript variant 1, mRNA”, gi|28559082|ref|NM_019857.3|[28559082]; 2184:
NM_019887, “Homo sapiens diablo homolog (Drosophila) (DIABLO), nuclear gene encoding”,
“mitochondrial protein, transcript variant 1, mRNA”, gi|42544195|ref|NM_019887.3|[42544195];
2185: NM_020062, “Homo sapiens SLC2A4 regulator (SLC2A4RG), mRNA”,
gi|39777592|ref|NM_020062.3|[39777592]; 2186: NM_020120, “Homo sapiens UDP-glucose
ceramide glucosyltransferase-like 1 (UGCGL1), mRNA”,
gi|9910279|ref|NM_020120.1|[9910279]; 2187: NM_020121, “Homo sapiens UDP-glucose
ceramide glucosyltransferase-like 2 (UGCGL2), mRNA”,
gi|11386200|ref|NM_020121.2|[11386200]; 2188: NM_020123, “Homo sapiens SM-11044
binding protein (SMBP), mRNA”, gi|33859832|ref|NM_020123.2|[33859832]; 2189:
NM_020126, “Homo sapiens sphingosine kinase 2 (SPHK2), mRNA”,
gi|21361698|ref|NM_020126.3|[21361698]; 2190: NM_020127, “Homo sapiens tuftelin 1
(TUFT1), mRNA”, gi|9910595|ref|NM_020127.1|[9910595]; 2191: NM_020130, “Homo
sapiens chromosome 8 open reading frame 4 (C8orf4), mRNA”,
gi|21359931|ref|NM_020130.2|[21359931]; 2192: NM_020133, Homo sapiens 1-acylglycerol-
3-phosphate O-acyltransferase 4 (lysophosphatidic, “acid acyltransferase, delta) (AGPAT4),
mRNA”, gi|9910391|ref|NM_020133.1|[9910391]; 2193: NM_020135, “Homo sapiens Werner
helicase interacting protein 1 (WRNIP1), transcript variant”, “1, mRNA”,
gi|18426901|ref|NM_020135.2|[18426901]; 2194: NM_020142, “Homo sapiens
NADH: ubiquinone oxidoreductase MLRQ subunit homolog (LOC56901),”, mRNA,
gi|34147589|ref|NM_020142.3|[34147589]; 2195: NM_020144, “Homo sapiens poly(A)
polymerase beta (testis specific) (PAPOLB), mRNA”,
gi|37202113|ref|NM_020144.3|[37202113]; 2196: NM_020147, “Homo sapiens THAP domain
containing 10 (THAP10), mRNA”, gi|31543086|ref|NM_020147.2|[31543086]; 2197:
NM_020151, “Homo sapiens START domain containing 7 (STARD7), transcript variant 1,
mRNA”, gi|21450854|ref|NM_020151.2|[21450854]; 2198: NM_020154, “Homo sapiens
chromosome 15 hypothetical ATG/GTP binding protein (LOC56851), mRNA”,
gi|9910345|ref|NM_020154.1|[9910345]; 2199: NM_020156, Homo sapiens core 1 UDP-
galactose: N-acetylgalactosamine-alpha-R beta, “1,3-galactosyltransferase (C1GALT1), mRNA”,
gi|9910143|ref|NM_020156.1|[9910143]; 2200: NM_020169, “Homo sapiens latexin protein
(LXN), mRNA”, gi|21359932|ref|NM_020169.2|[21359932]; 2201: NM_020170, “Homo
sapiens hypothetical protein from EUROIMAGE 2021883 (LOC56926), mRNA”,
gi|24308184|ref|NM_020170.1|[24308184]; 2202: NM_020184, “Homo sapiens cyclin M4
(CNNM4), mRNA”, gi|41350205|ref|NM_020184.2|[41350205]; 2203: NM_020186, “Homo
sapiens ACN9 homolog (S. cerevisiae) (ACN9), mRNA”,
gi|9910179|ref|NM_020186.1|[9910179]; 2204: NM_020188, “Homo sapiens DC13 protein
(DC13), mRNA”, gi|42476040|ref|NM_020188.2|[42476040]; 2205: NM_020189, “Homo
sapiens DC6 protein (DC6), mRNA”, gi|34222364|ref|NM_020189.4|[34222364]; 2206:
NM_020191, “Homo sapiens mitochondrial ribosomal protein S22 (MRPS22), nuclear gene
encoding”, “mitochondrial protein, mRNA”, gi|16554602|ref|NM_020191.2|[16554602]; 2207:
NM_020194, “Homo sapiens GL004 protein (GL004), mRNA”,
gi|31377606|ref|NM_020194.4|[31377606]; 2208: NM_020195, “Homo sapiens chromosome 14
open reading frame 124 (C14orf124), mRNA”, gi|9910257|ref|NM_020195.1|[9910257]; 2209:
NM_020196, “Homo sapiens XPA binding protein 2 (XAB2), mRNA”,
gi|9910259|ref|NM_020196.1|[9910259]; 2210: NM_020198, “Homo sapiens GK001 protein
(GK001), mRNA”, gi|9910241|ref|NM_020198.1|[9910241]; 2211: NM_020224,,
ref|NM_020224.1|[9910203], This record was temporarily removed by RefSeq staff for
additional review.,, 2212: NM_020226, “Homo sapiens PR domain containing 8 (PRDM8),
mRNA”, gi|41349479|ref|NM_020226.2|[41349479]; 2213: NM_020228, “Homo sapiens PR
domain containing 10 (PRDM10), transcript variant 1, mRNA”,
gi|41349457|ref|NM_020228.2|[41349457]; 2214: NM_020229, “Homo sapiens PR domain
containing 11 (PRDM11), mRNA”, gi|41349465|ref|NM_020229.2|[41349465]; 2215:
NM_020230, “Homo sapiens peter pan homolog (Drosophila) (PPAN), mRNA”,
gi|41872679|ref|NM_020230.3|[41872679]; 2216: NM_020231, “Homo sapiens x 010 protein
(MDS010), mRNA”, gi|34303962|ref|NM_020231.3|[34303962]; 2217: NM_020232, “Homo
sapiens hepatocellular carcinoma susceptibility protein (HCCA3), mRNA”,
gi|39725705|ref|NM_020232.3|[39725705]; 2218: NM_020233, “Homo sapiens x 006 protein
(MDS006), mRNA”, gi|37059747|ref|NM_020233.3|[37059747]; 2219: NM_020234, “Homo
sapiens x 009 protein (MDS009), mRNA”, gi|34222368|ref|NM_020234.3|[34222368]; 2220:
NM_020239, “Homo sapiens small protein effector 1 of Cdc42 (SPEC1), mRNA”,
gi|12965169|ref|NM_020239.2|[12965169]; 2221: NM_020243, Homo sapiens translocase of
outer mitochondrial membrane 22 homolog (yeast), “(TOMM22), mRNA”,
gi|39725679|ref|NM_020243.3|[39725679]; 2222: NM_020247, “Homo sapiens chaperone,
ABC1 activity of bc1 complex like (S. pombe) (CABC1),”, mRNA,
gi|34147521|ref|NM_020247.3|[34147521]; 2223: NM_020249, Homo sapiens a disintegrin-like
and metalloprotease (reprolysin type) with, “thrombospondin type 1 motif, 9 (ADAMTS9),
transcript variant 3, mRNA”, gi|33624884|ref|NM_020249.2|[33624884]; 2224: NM_020307,
“Homo sapiens cyclin L1 (CCNL1), mRNA”, gi|9945319|ref|NM_020307.1|[9945319]; 2225:
NM_020309, Homo sapiens solute carrier family 17 (sodium-dependent inorganic phosphate,
“cotransporter), member 7 (SLC17A7), mRNA”, gi|9945321|ref|NM_020309.1|[9945321]; 2226:
NM_020310, “Homo sapiens MAX binding protein (MNT), mRNA”,
gi|9945317|ref|NM_020310.1|[9945317]; 2227: NM_020319, “Homo sapiens hypothetical
protein DKFZp564O043 (DKFZP564O043), mRNA”,
gi|28461128|ref|NM_020319.1|[28461128]; 2228: NM_020354, “Homo sapiens ectonucleoside
triphosphate diphosphohydrolase 7 (ENTPD7), mRNA”,
gi|9966820|ref|NM_020354.1|[9966820]; 2229: NM_020357, “Homo sapiens PEST-containing
nuclear protein (PCNP), mRNA”, gi|9966826|ref|NM_020357.1|[9966826]; 2230: NM_020363,
“Homo sapiens deleted in azoospermia 2 (DAZ2), mRNA”,
gi|11036659|ref|NM_020363.1|[11036659]; 2231: NM_020367, “Homo sapiens chromosome 12
open reading frame 6 (C12orf6), mRNA”, gi|20127593|ref|NM_020367.2|[20127593]; 2232:
NM_020371, “Homo sapiens apoptosis, caspase activation inhibitor (AVEN), mRNA”,
gi|9966840|ref|NM_020371.1|[9966840]; 2233: NM_020375, “Homo sapiens chromosome 12
open reading frame 5 (C12orf5), mRNA”, gi|9966848|ref|NM_020375.1|[9966848]; 2234:
NM_020379, “Homo sapiens mannosidase, alpha, class 1C, member 1 (MAN1C1), mRNA”,
gi|9966902|ref|NM_020379.1|[9966902]; 2235: NM_020380, “Homo sapiens AF15q14 protein
(AF15Q14), mRNA”, gi|24475852|ref|NM_020380.2|[24475852]; 2236: NM_020381, “Homo
sapiens chromosome 6 open reading frame 210 (C6orf210), mRNA”,
gi|29893561|ref|NM_020381.2|[29893561]; 2237: NM_020387, “Homo sapiens RAB25,
member RAS oncogene family (RAB25), mRNA”, gi|9966860|ref|NM_020387.1|[9966860];
2238: NM_020397, “Homo sapiens calcium/calmodulin-dependent protein kinase ID
(CAMK1D), mRNA”, gi|9966874|ref|NM_020397.1|[9966874]; 2239: NM_020401, “Homo
sapiens nuclear pore complex protein (NUP107), mRNA”,
gi|9966880|ref|NM_020401.1|[9966880]; 2240: NM_020410, “Homo sapiens ATPase type 13A
(ATP13A), mRNA”, gi|9966896|ref|NM_020410.1|[9966896]; 2241: NM_020418, “Homo
sapiens poly(rC) binding protein 4 (PCBP4), transcript variant 1, mRNA”,
gi|14670367|ref|NM_020418.2|[14670367]; 2242: NM_020423, “Homo sapiens ezrin-binding
partner PACE-1 (PACE-1), transcript variant 1, mRNA”,
gi|27363466|ref|NM_020423.4|[27363466]; 2243: NM_020424, “Homo sapiens hypothetical
protein A-211C6.1 (LOC57149), mRNA”, gi|19923825|ref|NM_020424.2|[19923825]; 2244:
NM_020433, “Homo sapiens junctophilin 2 (JPH2), transcript variant 1, mRNA”,
gi|29893810|ref|NM_020433.3|[29893810]; 2245: NM_020453, “Homo sapiens ATPase, Class
V, type 10D (ATP10D), mRNA”, gi|28466988|ref|NM_020453.2|[28466988]; 2246:
NM_020465, “Homo sapiens NDRG family member 4 (NDRG4), mRNA”,
gi|14165263|ref|NM_020465.1|[14165263]; 2247: NM_020466, “Homo sapiens hypothetical
protein dJ122O8.2 (DJ122O8.2), mRNA”, gi|20070310|ref|NM_020466.3|[20070310]; 2248:
NM_020529, Homo sapiens nuclear factor of kappa light polypeptide gene enhancer in B-cells,
“inhibitor, alpha (NFKBIA), mRNA”, gi|10092618|ref|NM_020529.1|[10092618]; 2249:
NM_020533, “Homo sapiens mucolipin 1 (MCOLN1), mRNA”,
gi|10092596|ref|NM_020533.1|[10092596]; 2250: NM_020549, “Homo sapiens choline
acetyltransferase (CHAT), transcript variant M, mRNA”,
gi|11038626|ref|NM_020549.2|[11038626]; 2251: NM_020638, “Homo sapiens fibroblast
growth factor 23 (FGF23), mRNA”, gi|15055547|ref|NM_020638.2|[15055547]; 2252:
NM_020639, “Homo sapiens ankyrin repeat domain 3 (ANKRD3), mRNA”,
gi|41327753|ref|NM_020639.2|[41327753]; 2253: NM_020640, “Homo sapiens RP42 homolog
(RP42), mRNA”, gi|36030882|ref|NM_020640.2|[36030882]; 2254: NM_020642, “Homo
sapiens chromosome 11 open reading frame 17 (C11orf17), transcript variant”, “2, mRNA”,
gi|21361869|ref|NM_020642.2|[21361869]; 2255: NM_020644, “Homo sapiens chromosome 11
open reading frame 15 (C11orf15), mRNA”, gi|11034854|ref|NM_020644.1|[11034854]; 2256:
NM_020645, “Homo sapiens nuclear receptor interacting protein 3 (NRIP3), mRNA”,
gi|11034818|ref|NM_020645.1|[11034818]; 2257: NM_020648, “Homo sapiens twisted
gastrulation homolog 1 (Drosophila) (TWSG1), mRNA”,
gi|21314788|ref|NM_020648.3|[21314788]; 2258: NM_020649, “Homo sapiens chromobox
homolog 8 (Pc class homolog, Drosophila) (CBX8), mRNA”,
gi|10190681|ref|NM_020649.1|[10190681]; 2259: NM_020655, “Homo sapiens junctophilin 3
(JPH3), mRNA”, gi|21704282|ref|NM_020655.2|[21704282]; 2260: NM_020669,,
ref|NM_020669.1|[10190709], This record was temporarily removed by RefSeq staff for
additional review.,, 2261: NM_020673, “Homo sapiens RAB22A, member RAS oncogene
family (RAB22A), mRNA”, gi|34577103|ref|NM_020673.2|[34577103]; 2262: NM_020685,
“Homo sapiens HT021 (HT021), mRNA”, gi|34222336|ref|NM_020685.3|[34222336]; 2263:
NM_020710, “Homo sapiens KIAA1185 protein (KIAA1185), mRNA”,
gi|24308206|ref|NM_020710.1|[24308206]; 2264: NM_020826, “Homo sapiens synaptotagmin
XIII (SYT13), mRNA”, gi|24308232|ref|NM_020826.1|[24308232]; 2265: NM_020836, “Homo
sapiens brain-enriched guanylate kinase-associated protein (KIAA1446), mRNA”,
gi|34147339|ref|NM_020836.2|[34147339]; 2266: NM_020858, “Homo sapiens sema domain,
transmembrane domain (TM), and cytoplasmic domain,”, “(semaphorin) 6D (SEMA6D),
transcript variant 1, mRNA”, gi|24234728|ref|NM_020858.1|[24234728]; 2267: NM_020892,
“Homo sapiens deltex homolog 2 (Drosophila) (DTX2), mRNA”,
gi|24308252|ref|NM_020892.1|[24308252]; 2268: NM_020898, “Homo sapiens KIAA1536
protein (KIAA1536), mRNA”, gi|14149741|ref|NM_020898.1|[14149741]; 2269: NM_020904,
“Homo sapiens pleckstrin homology domain containing, family A (phosphoinositide”, “binding
specific) member 4 (PLEKHA4), mRNA”, gi|10190743|ref|NM_020904.1|[10190743]; 2270:
NM_020982,, ref|NM_020982.2|[44680149]; 2271: NM_020998, “Homo sapiens macrophage
stimulating 1 (hepatocyte growth factor-like) (MST1),”, mRNA,
gi|31543211|ref|NM_020998.2|[31543211]; 2272: NM_020999, “Homo sapiens neurogenin 3
(NEUROG3), mRNA”, gi|10337610|ref|NM_020999.1|[10337610]; 2273: NM_021018, “Homo
sapiens histone 1, H3f (HIST1H3F), mRNA”, gi|21396497|ref|NM_021018.2|[21396497]; 2274:
NM_021025, “Homo sapiens T-cell leukemia, homeobox 3 (TLX3), mRNA”,
gi|10440563|ref|NM_021025.1|[10440563]; 2275: NM_021062, “Homo sapiens histone 1, H2bb
(HIST1H2BB), mRNA”, gi|19924303|ref|NM_021062.2|[19924303]; 2276: NM_021070,
“Homo sapiens latent transforming growth factor beta binding protein 3 (LTBP3),”, mRNA,
gi|18497287|ref|NM_021070.2|[18497287]; 2277: NM_021077, “Homo sapiens neuromedin B
(NMB), mRNA”, gi|24475648|ref|NM_021077.2|[24475648]; 2278: NM_021080, “Homo
sapiens disabled homolog 1 (Drosophila) (DAB1), mRNA”,
gi|33350927|ref|NM_021080.3|[33350927]; 2279: NM_021081, “Homo sapiens growth
hormone releasing hormone (GHRH), mRNA”, gi|3058116|ref|NM_021081.3|[30581161];
2280: NM_021098, “Homo sapiens calcium channel, voltage-dependent, alpha 1H subunit
(CACNA1H),”, mRNA, gi|10864076|ref|NM_021098.1|[10864076]; 2281: NM_021100, “Homo
sapiens NFS1 nitrogen fixation 1 (S. cerevisiae) (NFS1), nuclear gene”, “encoding mitochondrial
protein, transcript variant 1, mRNA”, gi|32307131|ref|NM_021100.3|[32307131]; 2282:
NM_021104, “Homo sapiens ribosomal protein L41 (RPL41), mRNA”,
gi|10863874|ref|NM_021104.1|[10863874]; 2283: NM_021126, “Homo sapiens
mercaptopyruvate sulfurtransferase (MPST), mRNA”,
gi|23510449|ref|NM_021126.3|[23510449]; 2284: NM_021133, “Homo sapiens ribonuclease L
(2′,5′-oligoisoadenylate synthetase-dependent)”, “(RNASEL), mRNA”,
gi|30795246|ref|NM_021133.2|[30795246]; 2285: NM_021134, “Homo sapiens mitochondrial
ribosomal protein L23 (MRPL23), nuclear gene encoding”, “mitochondrial protein, mRNA”,
gi|27436903|ref|NM_021134.2|[27436903]; 2286: NM_021147, “Homo sapiens uracil-DNA
glycosylase 2 (UNG2), mRNA”, gi|10863950|ref|NM_021147.1|[10863950]; 2287: NM_021149,
“Homo sapiens coactosin-like 1 (Dictyostelium) (COTL1), mRNA”,
gi|23510452|ref|NM_021149.2|[23510452]; 2288: NM_021158, “Homo sapiens chromosome 20
open reading frame 97 (C20orf97), mRNA”, gi|41327717|ref|NM_021158.3|[41327717]; 2289:
NM_021161, “Homo sapiens potassium channel, subfamily K, member 10 (KCNK10),
transcript”, “variant 1, mRNA”, gi|20143942|ref|NM_021161.3|[20143942]; 2290: NM_021165,
“Homo sapiens hypothetical protein from clone 24828 (LOC57795), mRNA”,
gi|23943865|ref|NM_021165.1|[23943865]; 2291: NM_021168, “Homo sapiens RAB40C,
member RAS oncogene family (RAB40C), mRNA”, gi|18373307|ref|NM_021168.1|[18373307];
2292: NM_021174, “Homo sapiens p30 DBC protein (DBC-1), transcript variant 1, mRNA”,
gi|40548406|ref|NM_021174.4|[40548406]; 2293: NM_021184, “Homo sapiens chromosome 6
open reading frame 47 (C6orf47), mRNA”, gi|10863984|ref|NM_021184.1|[10863984]; 2294:
NM_021187, “Homo sapiens cytochrome P450, family 4, subfamily F, polypeptide 11
(CYP4F11),”, mRNA, gi|10863992|ref|NM_021187.1|[10863992]; 2295: NM_021193, “Homo
sapiens homeo box D12 (HOXD12), mRNA”, gi|23510369|ref|NM_021193.2|[23510369]; 2296:
NM_021195, “Homo sapiens claudin 6 (CLDN6), mRNA”,
gi|39725680|ref|NM_021195.3|[39725680]; 2297: NM_021199, “Homo sapiens sulfide quinone
reductase-like (yeast) (SQRDL), mRNA”, gi|10864010|ref|NM_021199.1|[10864010]; 2298:
NM_021204, “Homo sapiens E-1 enzyme (MASA), mRNA”,
gi|10864016|ref|NM_021204.1|[10864016]; 2299: NM_021208, “Homo sapiens chromosome 9
open reading frame 27 (C9orf27), mRNA”, gi|10864018|ref|NM_021208.1|[10864018]; 2300:
NM_021211, “Homo sapiens transposon-derived Buster1 transposase-like protein
(LOC58486),”, mRNA, gi|10864022|ref|NM_021211.1|[10864022]; 2301: NM_021226, “Homo
sapiens Rho GTPase activating protein 22 (ARHGAP22), mRNA”,
gi|34013589|ref|NM_021226.2|[34013589]; 2302: NM_021238, “Homo sapiens chromosome 12
open reading frame 14 (C12orf14), mRNA”, gi|10864048|ref|NM_021238.1|[10864048]; 2303:
NM_021242, “Homo sapiens hypothetical protein STRAIT11499 (STRAIT11499), mRNA”,
gi|39725681|ref|NM_021242.3|[39725681]; 2304: NM_021249, “Homo sapiens sorting nexin 6
(SNX6), transcript variant 1, mRNA”, gi|23111048|ref|NM_021249.2|[23111048]; 2305:
NM_021257, “Homo sapiens neuroglobin (NGB), mRNA”,
gi|21361878|ref|NM_021257.2|[21361878]; 2306: NM_021258, “Homo sapiens interleukin 22
receptor, alpha 1 (IL22RA1), mRNA”, gi|31317238|ref|NM_021258.2|[31317238]; 2307:
NM_021259, “Homo sapiens transmembrane protein 8 (five membrane-spanning domains)
(TMEM8),”, mRNA, gi|10864068|ref|NM_021259.1|[10864068]; 2308: NM_021614, “Homo
sapiens potassium intermediate/small conductance calcium-activated channel,”, “subfamily N,
member 2 (KCNN2), transcript variant 1, mRNA”, gi|25777644|ref|NM_021614.2|[25777644];
2309: NM_021620, “Homo sapiens PR domain containing 13 (PRDM13), mRNA”,
gi|41349467|ref|NM_021620.2|[41349467]; 2310: NM_021625, “Homo sapiens transient
receptor potential cation channel, subfamily V, member 4”, “(TRPV4), transcript variant 1,
mRNA”, gi|22547183|ref|NM_021625.3|[22547183]; 2311: NM_021627, “Homo sapiens
sentrin-specific protease (SENP2), mRNA”, gi|11055993|ref|NM_021627.1|[11055993]; 2312:
NM_021633, “Homo sapiens kelch-like 12 (Drosophila) (KLHL12), mRNA”,
gi|21361889|ref|NM_021633.2|[21361889]; 2313: NM_021640, “Homo sapiens chromosome 12
open reading frame 10 (C12orf10), mRNA”, gi|11056017|ref|NM_021640.1|[11056017]; 2314:
NM_021729, “Homo sapiens vacuolar protein sorting 11 (yeast) (VPS11), mRNA”,
gi|17978476|ref|NM_021729.3|[17978476]; 2315: NM_021812, “Homo sapiens
blepharophimosis, epicanthus inversus and ptosis, candidate 1”, “(BPESC1), mRNA”,
gi|11141882|ref|NM_021812.1|[11141882]; 2316: NM_021813, “Homo sapiens BTB and CNC
homology 1, basic leucine zipper transcription factor 2”, “(BACH2), mRNA”,
gi|13540489|ref|NM_021813.1|[13540489]; 2317: NM_021817, “Homo sapiens brain link
protein-1 (BRAL1), mRNA”, gi|11141886|ref|NM_021817.1|[11141886]; 2318: NM_021818,
“Homo sapiens salvador homolog 1 (Drosophila) (SAV1), mRNA”,
gi|18860913|ref|NM_021818.2|[18860913]; 2319: NM_021820, “Homo sapiens chromosome 6
open reading frame 75 (C6orf75), mRNA”, gi|11141892|ref|NM_021820.1|[11141892]; 2320:
NM_021823, “Homo sapiens hypothetical protein MDS018 (MDS018), mRNA”,
gi|21361899|ref|NM_021823.2|[21361899]; 2321: NM_021824, “Homo sapiens NIF3 NGG1
interacting factor 3-like 1 (S. pombe) (NIF3L1), mRNA”,
gi|11141898|ref|NM_021824.1|[11141898]; 2322: NM_021826, “Homo sapiens hypothetical
protein FLJ13149 (FLJ13149), mRNA”, gi|40806183|ref|NM_021826.4|[40806183]; 2323:
NM_021828, “Homo sapiens heparanase 2 (HPSE2), mRNA”,
gi|40254951|ref|NM_021828.2|[40254951]; 2324: NM_021830, “Homo sapiens progressive
external ophthalmoplegia 1 (PEO1), mRNA”, gi|39725941|ref|NM_021830.3|[39725941]; 2325:
NM_021831, “Homo sapiens hypothetical protein FLJ21839 (FLJ21839), mRNA”,
gi|34147509|ref|NM_021831.3|[34147509]; 2326: NM_021833, “Homo sapiens uncoupling
protein 1 (mitochondrial, proton carrier) (UCP1),”, “nuclear gene encoding mitochondrial
protein, mRNA”, gi|21614550|ref|NM_021833.3|[21614550]; 2327: NM_021926, “Homo
sapiens aristaless-like homeobox 4 (ALX4), mRNA”,
gi|11496266|ref|NM_021926.1|[11496266]; 2328: NM_021932, “Homo sapiens likely ortholog
of mouse synembryn (RIC-8), mRNA”, gi|27883865|ref|NM_021932.4|[27883865]; 2329:
NM_021933, “Homo sapiens hypothetical protein FLJ12438 (FLJ12438), mRNA”,
gi|11345471|ref|NM_021933.1|[11345471]; 2330: NM_021934, “Homo sapiens hypothetical
protein FLJ11773 (FLJ11773), mRNA”, gi|34222337|ref|NM_021934.3|[34222337]; 2331:
NM_021939, “Homo sapiens FK506 binding protein 10, 65 kDa (FKBP10), mRNA”,
gi|21361894|ref|NM_021939.2|[21361894]; 2332: NM_021940, “Homo sapiens stromal
membrane-associated protein 1 (SMAP1), mRNA”, gi|21264557|ref|NM_021940.2|[21264557];
2333: NM_021943, “Homo sapiens testis expressed sequence 27 (TEX27), mRNA”,
gi|11345483|ref|NM_021943.1|[11345483]; 2334: NM_021946, “Homo sapiens hypothetical
protein FLJ11362 (FLJ11362), mRNA”, gi|33286441|ref|NM_021946.2|[33286441]; 2335:
NM_021958, “Homo sapiens H2.0-like homeo box 1 (Drosophila) (HLX1), mRNA”,
gi|19923769|ref|NM_021958.2|[19923769]; 2336: NM_021959, “Homo sapiens protein
phosphatase 1, regulatory (inhibitor) subunit 11 (PPP1R11),”, “transcript variant 1, mRNA”,
gi|11386174|ref|NM_021959.1|[11386174]; 2337: NM_021961, Homo sapiens TEA domain
family member 1 (SV40 transcriptional enhancer factor), “(TEAD1), mRNA”,
gi|38570152|ref|NM_021961.2|[38570152]; 2338: NM_021970, Homo sapiens mitogen-
activated protein kinase kinase 1 interacting protein 1, “(MAP2K1IP1), mRNA”,
gi|21614526|ref|NM_021970.2|[21614526]; 2339: NM_021972, “Homo sapiens sphingosine
kinase 1 (SPHK1), mRNA”, gi|21361087|ref|NM_021972.2|[21361087]; 2340: NM_021974,
“Homo sapiens polymerase (RNA) II (DNA directed) polypeptide F (POLR2F), mRNA”,
gi|14602451|ref|NM_021974.2|[14602451]; 2341: NM_022003, “Homo sapiens FXYD domain
containing ion transport regulator 6 (FXYD6), mRNA”,
gi|11612654|ref|NM_022003.1|[11612654]; 2342: NM_022039, “Homo sapiens split hand/foot
malformation (ectrodactyly) type 3 (SHFM3), mRNA”,
gi|24475655|ref|NM_022039.2|[24475655]; 2343: NM_022041, “Homo sapiens giant axonal
neuropathy (gigaxonin) (GAN), mRNA”, gi|21614518|ref|NM_022041.2|[21614518]; 2344:
NM_022042, “Homo sapiens solute carrier family 26 (sulfate transporter), member 1
(SLC26A1),”, “transcript variant 1, mRNA”, gi|20336271|ref|NM_022042.2|[20336271]; 2345:
NM_022044, “Homo sapiens stromal cell-derived factor 2-like 1 (SDF2L1), mRNA”,
gi|11545742|ref|NM_022044.1|[11545742]; 2346: NM_022049, “Homo sapiens G-protein
coupled receptor 88 (GPR88), mRNA”, gi|11545752|ref|NM_022049.1|[11545752]; 2347:
NM_022054, “Homo sapiens potassium channel, subfamily K, member 13 (KCNK13), mRNA”,
gi|16306554|ref|NM_022054.2|[16306554]; 2348: NM_022063, “Homo sapiens hypothetical
protein FLJ13188 (FLJ13188), mRNA”, gi|11545770|ref|NM_022063.1|[11545770]; 2349:
NM_022064, “Homo sapiens ring finger protein 123 (RNF123), mRNA”,
gi|37588868|ref|NM_022064.2|[37588868]; 2350: NM_022067, “Homo sapiens chromosome 14
open reading frame 133 (C14orf133), mRNA”, gi|20127606|ref|NM_022067.2|[20127606];
2351: NM_022071, “Homo sapiens hypothetical protein FLJ20967 (FLJ20967), mRNA”,
gi|21361890|ref|NM_022071.2|[21361890]; 2352: NM_022072, “Homo sapiens hypothetical
protein FLJ22609 (FLJ22609), mRNA”, gi|31542738|ref|NM_022072.2|[31542738]; 2353:
NM_022082, “Homo sapiens chromosome 20 open reading frame 59 (C20orf59), mRNA”,
gi|31542262|ref|NM_022082.2|[31542262]; 2354: NM_022089, “Homo sapiens putative
ATPase (HSA9947), mRNA”, gi|13435128|ref|NM_022089.1|[13435128]; 2355: NM_022096,
“Homo sapiens ankyrin repeat domain 5 (ANKRD5), transcript variant 1, mRNA”,
gi|38569425|ref|NM_022096.4|[38569425]; 2356: NM_022097, “Homo sapiens hepatocellular
carcinoma antigen gene 520 (LOC63928), mRNA”, gi|11545810|ref|NM_022097.1|[11545810];
2357: NM_022098, “Homo sapiens hypothetical protein LOC63929 (LOC63929), mRNA”,
gi|38195085|ref|NM_022098.2|[38195085]; 2358: NM_022101, “Homo sapiens hypothetical
protein FLJ22965 (FLJ22965), mRNA”, gi|34147219|ref|NM_022101.2|[34147219]; 2359:
NM_022111, “Homo sapiens claspin homolog (Xenopus laevis) (CLSPN), mRNA”,
gi|21735568|ref|NM_022111.2|[21735568]; 2360: NM_022114, “Homo sapiens PR domain
containing 16 (PRDM16), transcript variant 1, mRNA”,
gi|41349469|ref|NM_022114.2|[41349469]; 2361: NM_022118, “Homo sapiens chromosome 13
open reading frame 10 (C13orf10), mRNA”, gi|31652263|ref|NM_022118.3|[31652263]; 2362:
NM_022120, “Homo sapiens 3-oxoacid CoA transferase 2 (OXCT2), mRNA”,
gi|11545840|ref|NM_022120.1|[11545840]; 2363: NM_022121, “Homo sapiens PERP, TP53
apoptosis effector (PERP), mRNA”, gi|31377721|ref|NM_022121.2|[31377721]; 2364:
NM_022126, Homo sapiens phospholysine phosphohistidine inorganic pyrophosphate
phosphatase, “(LHPP), mRNA”, gi|33636765|ref|NM_022126.2|[33636765]; 2365: NM_022130,
“Homo sapiens golgi phosphoprotein 3 (coat-protein) (GOLPH3), mRNA”,
gi|29550859|ref|NM_022130.3|[29550859]; 2366: NM_022133, “Homo sapiens sorting nexin 16
(SNX16), transcript variant 1, mRNA”, gi|23238243|ref|NM_022133.2|[23238243]; 2367:
NM_022135, “Homo sapiens popeye domain containing 2 (POPDC2), mRNA”,
gi|22209003|ref|NM_022135.2|[22209003]; 2368: NM_022149, “Homo sapiens melanoma
antigen, family F, 1 (MAGEF1), mRNA”, gi|34335240|ref|NM_022149.3|[34335240]; 2369:
NM_022151, “Homo sapiens modulator of apoptosis 1 (MOAP1), mRNA”,
gi|21536456|ref|NM_022151.3|[21536456]; 2370: NM_022156, “Homo sapiens PP3111 protein
(PP3111), mRNA”, gi|40807365|ref|NM_022156.3|[40807365]; 2371: NM_022157, “Homo
sapiens Ras-related GTP binding C (RRAGC), mRNA”,
gi|31542866|ref|NM_022157.2|[31542866]; 2372: NM_022158, “Homo sapiens fructosamine-3-
kinase (FN3K), mRNA”, gi|31542792|ref|NM_022158.2|[31542792]; 2373: NM_022164,
“Homo sapiens lipocalin 7 (LCN7), mRNA”, gi|11545917|ref|NM_022164.1|[11545917]; 2374:
NM_022171, “Homo sapiens T-cell leukemia translocation altered gene (TCTA), mRNA”,
gi|11560140|ref|NM_022171.1|[11560140]; 2375: NM_022341, “Homo sapiens peptide
deformylase-like protein (PDF), mRNA”, gi|11641242|ref|NM_022341.1|[11641242]; 2376:
NM_022353, “Homo sapiens O-sialoglycoprotein endopeptidase-like 1 (OSGEPL1), mRNA”,
gi|11641264|ref|NM_022353.1|[11641264]; 2377: NM_022354, “Homo sapiens
spermatogenesis associated 1 (SPATA1), mRNA”, gi|11641266|ref|NM_022354.1|[11641266];
2378: NM_022356, “Homo sapiens leucine proline-enriched proteoglycan (leprecan) 1
(LEPRE1), mRNA”, gi|21361917|ref|NM_022356.2|[21361917]; 2379: NM_022362, “Homo
sapiens MMS19-like (MET18 homolog, S. cerevisiae) (MMS19L), mRNA”,
gi|31543206|ref|NM_022362.2|[31543206]; 2380: NM_022365, “Homo sapiens DnaJ (Hsp40)
homolog, subfamily C, member 1 (DNAJC1), mRNA”,
gi|21361911|ref|NM_022365.2|[21361911]; 2381: NM_022366, “Homo sapiens transcription
factor B2, mitochondrial (TFB2M), mRNA”, gi|11641288|ref|NM_022366.1|[11641288]; 2382:
NM_022367, “Homo sapiens hypothetical protein FLJ12287 similar to semaphorins
(FLJ12287),”, mRNA, gi|21361913|ref|NM_022367.2|[21361913]; 2383: NM_022450, “Homo
sapiens rhomboid family 1 (Drosophila) (RHBDP1), mRNA”,
gi|21359942|ref|NM_022450.2|[21359942]; 2384: NM_022451, “Homo sapiens AD24 protein
(AD24), mRNA”, gi|31377626|ref|NM_022451.9|[31377626]; 2385: NM_022452, “Homo
sapiens fibrosin 1 (FBS1), mRNA”, gi|11967986|ref|NM_022452.1|[11967986]; 2386:
NM_022460, “Homo sapiens HS1-binding protein 3 (FLJ14249), transcript variant 1, mRNA”,
gi|18491011|ref|NM_022460.2|[18491011]; 2387: NM_022461, “Homo sapiens 5-azacytidine
induced gene 2 (AZ2), transcript variant 1, mRNA”, gi|42716307|ref|NM_022461.2|[42716307];
2388: NM_022470, “Homo sapiens p53 target zinc finger protein (WIG1), transcript variant 1,
mRNA”, gi|23199979|ref|NM_022470.2|[23199979]; 2389: NM_022474, “Homo sapiens
membrane protein, palmitoylated 5 (MAGUK p55 subfamily member 5)”, “(MPP5), mRNA”,
gi|38570141|ref|NM_022474.2|[38570141]; 2390: NM_022476, “Homo sapiens fused toes
homolog (mouse) (FTS), mRNA”, gi|11968026|ref|NM_022476.1|[11968026]; 2391:
NM_022484, “Homo sapiens hypothetical protein FLJ13576 (FLJ13576), mRNA”,
gi|21362101|ref|NM_022484.2|[21362101]; 2392: NM_022485, “Homo sapiens hypothetical
protein FLJ22405 (FLJ22405), mRNA”, gi|20127610|ref|NM_022485.2|[20127610]; 2393:
NM_022494, “Homo sapiens zinc finger, DHHC domain containing 6 (ZDHHC6), mRNA”,
gi|11968052|ref|NM_022494.1|[11968052]; 2394: NM_022496, “Homo sapiens actin-related
protein 6 (ACTR6), mRNA”, gi|31541858|ref|NM_022496.2|[31541858]; 2395: NM_022551,
“Homo sapiens ribosomal protein S18 (RPS18), mRNA”,
gi|14165467|ref|NM_022551.2|[14165467]; 2396: NM_022553, “Homo sapiens vacuolar
protein sorting 52 (yeast) (VPS52), transcript variant 2,”, mRNA,
gi|18379339|ref|NM_022553.3|[18379339]; 2397: NM_022658, “Homo sapiens homeo box C8
(HOXC8), mRNA”, gi|24497545|ref|NM_022658.2|[24497545]; 2398: NM_022659, “Homo
sapiens early B-cell factor 2 (EBF2), mRNA”, gi|12056972|ref|NM_022659.1|[12056972]; 2399:
NM_022662, “Homo sapiens anaphase promoting complex subunit 1 (ANAPC1), mRNA”,
gi|12056970|ref|NM_022662.1|[12056970]; 2400: NM_022725, “Homo sapiens Fanconi
anemia, complementation group F (FANCF), mRNA”,
gi|42716285|ref|NM_022725.2|[42716285]; 2401: NM_022726, “Homo sapiens elongation of
very long chain fatty acids (FEN1/Elo2, SUR4/Elo3,”, “yeast)-like 4 (ELOVL4), mRNA”,
gi|21362099|ref|NM_022726.2|[21362099]; 2402: NM_022727, “Homo sapiens HpaII tiny
fragments locus 9C (HTF9C), transcript variant 2, mRNA”,
gi|21361611|ref|NM_022727.3|[21361611]; 2403: NM_022730, “Homo sapiens COP9
constitutive photomorphogenic homolog subunit 7B (Arabidopsis), “(COPS7B), mRNA”,
gi|12232384|ref|NM_022730.1|[12232384]; 2404: NM_022746, “Homo sapiens hypothetical
protein FLJ22390 (FLJ22390), mRNA”, gi|33285009|ref|NM_022746.2|[33285009]; 2405:
NM_022750, “Homo sapiens zinc finger CCCH type domain containing 1 (ZC3HDC1),
mRNA”, gi|12232412|ref|NM_022750.1|[12232412]; 2406: NM_022754, “Homo sapiens
sideroflexin 1 (SFXN1), mRNA”, gi|40255158|ref|NM_022754.4|[40255158]; 2407:
NM_022756, “Homo sapiens hypothetical protein FLJ11730 (FLJ11730), mRNA”,
gi|40255019|ref|NM_022756.3|[40255019]; 2408: NM_022761, “Homo sapiens chromosome 11
open reading frame 1 (C11orf1), mRNA”, gi|12232430|ref|NM_022761.1|[12232430]; 2409:
NM_022762, “Homo sapiens hypothetical protein FLJ22318 (FLJ22318), mRNA”,
gi|34147687|ref|NM_022762.3|[34147687]; 2410: NM_022765, Homo sapiens NEDD9
interacting protein with calponin homology and LIM domains, “(NICAL), mRNA”,
gi|20127615|ref|NM_022765.2|[20127615]; 2411: NM_022766, “Homo sapiens ceramide kinase
(CERK), transcript variant 1, mRNA”, gi|32967301|ref|NM_022766.4|[32967301]; 2412:
NM_022776, “Homo sapiens oxysterol binding protein-like 11 (OSBPL11), mRNA”,
gi|23111058|ref|NM_022776.3|[23111058]; 2413: NM_022781, “Homo sapiens ring finger
protein 38 (RNF38), transcript variant 1, mRNA”, gi|37577174|ref|NM_022781.3|[37577174];
2414: NM_022784, “Homo sapiens hypothetical protein FLJ12476 (FLJ12476), mRNA”,
gi|12232474|ref|NM_022784.1|[12232474]; 2415: NM_022785, “Homo sapiens CAP-binding
protein complex interacting protein 1 (FLJ23588),”, “transcript variant 1, mRNA”,
gi|38570106|ref|NM_022785.2|[38570106]; 2416: NM_022819, “Homo sapiens phospholipase
A2, group IIF (PLA2G2F), mRNA”, gi|12383057|ref|NM_022819.1|[12383057]; 2417:
NM_022834, “Homo sapiens von Willebrand factor A domain-related protein (WARP),
transcript”, “variant 1, mRNA”, gi|40068484|ref|NM_022834.3|[40068484]; 2418: NM_022836,
“Homo sapiens DNA cross-link repair 1B (PSO2 homolog, S. cerevisiae) (DCLRE1B),”, mRNA,
gi|24431998|ref|NM_022836.2|[24431998]; 2419: NM_022840, “Homo sapiens
methyltransferase like 4 (METTL4), mRNA”, gi|38505223|ref|NM_022840.2|[38505223]; 2420:
NM_022897, “Homo sapiens RAN binding protein 17 (RANBP17), mRNA”,
gi|22095364|ref|NM_022897.2|[22095364]; 2421: NM_022898, “Homo sapiens B-cell
CLL/lymphoma 11B (zinc finger protein) (BCL11B), transcript”, “variant 2, mRNA”,
gi|12597634|ref|NM_022898.1|[12597634]; 2422: NM_022899, “Homo sapiens ARP8 actin-
related protein 8 homolog (yeast) (ACTR8), mRNA”,
gi|39812114|ref|NM_022899.3|[39812114]; 2423: NM_022903, “Homo sapiens hypothetical
protein FLJ12800 (FLJ12800), mRNA”, gi|33285012|ref|NM_022903.2|[33285012]; 2424:
NM_022908, “Homo sapiens hypothetical protein FLJ12442 (FLJ12442), mRNA”,
gi|12597652|ref|NM_022908.1|[12597652]; 2425: NM_022911, “Homo sapiens solute carrier
family 26, member 6 (SLC26A6), transcript variant 1,”, mRNA,
gi|20336275|ref|NM_022911.2|[20336275]; 2426: NM_022914, “Homo sapiens hypothetical
protein 24432 (24432), mRNA”, gi|12597658|ref|NM_022914.1|[12597658]; 2427: NM_022917,
“Homo sapiens nucleolar protein family 6 (RNA-associated) (NOL6), transcript”, “variant
alpha, mRNA”, gi|39777587|ref|NM_022917.4|[39777587]; 2428: NM_023008, “Homo sapiens
hypothetical protein FLJ12949 (FLJ12949), transcript variant 1,”, mRNA,
gi|30410782|ref|NM_023008.2|[30410782]; 2429: NM_023039, “Homo sapiens ankyrin repeat,
family A (RFXANK-like), 2 (ANKRA2), mRNA”, gi|21362082|ref|NM_023039.2|[21362082];
2430: NM_023067, “Homo sapiens forkhead box L2 (FOXL2), mRNA”,
gi|42716284|ref|NM_023067.2|[42716284]; 2431: NM_023071, “Homo sapiens
spermatogenesis associated, serine-rich 2 (SPATS2), mRNA”,
gi|12751480|ref|NM_023071.1|[12751480]; 2432: NM_023918, “Homo sapiens taste receptor,
type 2, member 8 (TAS2R8), mRNA”, gi|12965173|ref|NM_023918.1|[12965173]; 2433:
NM_023921, “Homo sapiens taste receptor, type 2, member 10 (TAS2R10), mRNA”,
gi|12965179|ref|NM_023921.1|[12965179]; 2434: NM_023922, “Homo sapiens taste receptor,
type 2, member 14 (TAS2R14), mRNA”, gi|12965181|ref|NM_023922.1|[12965181]; 2435:
NM_023924, “Homo sapiens bromodomain containing 9 (BRD9), mRNA”,
gi|12965190|ref|NM_023924.1|[12965190]; 2436: NM_023925, “Homo sapiens C1q domain
containing 1 (C1QDC1), transcript variant L, mRNA”,
gi|23503234|ref|NM_023925.2|[23503234]; 2437: NM_023927, “Homo sapiens HCV NS3-
transactivated protein 2 (NS3TP2), mRNA”, gi|12965196|ref|NM_023927.1|[12965196]; 2438:
NM_023932, “Homo sapiens EGF-like-domain, multiple 9 (EGFL9), mRNA”,
gi|13027595|ref|NM_023932.1|[13027595]; 2439: NM_023933, “Homo sapiens hypothetical
protein MGC2494 (MGC2494), mRNA”, gi|13027599|ref|NM_023933.1|[13027599]; 2440:
NM_023936, “Homo sapiens mitochondrial ribosomal protein S34 (MRPS34), nuclear gene
encoding”, “mitochondrial protein, mRNA”, gi|13027603|ref|NM_023936.1|[13027603]; 2441:
NM_023938, “Homo sapiens specifically androgen-regulated protein (SARG), mRNA”,
gi|40556373|ref|NM_023938.3|[40556373]; 2442: NM_023944, “Homo sapiens cytochrome
P450, family 4, subfamily F, polypeptide 12 (CYP4F12),”, mRNA,
gi|13184045|ref|NM_023944.1|[13184045]; 2443: NM_024032, “Homo sapiens hypothetical
protein MGC3130 (MGC3130), mRNA”, gi|31543178|ref|NM_024032.2|[31543178]; 2444:
NM_024034, Homo sapiens ganglioside-induced differentiation-associated protein 1-like 1,
“(GDAP1L1), mRNA”, gi|30581159|ref|NM_024034.3|[30581159]; 2445: NM_024040, “Homo
sapiens chromosome 10 open reading frame 66 (C10orf66), mRNA”,
gi|13128995|ref|NM_024040.1|[13128995]; 2446: NM_024041, “Homo sapiens sodium channel
modifier 1 (SCNM1), mRNA”, gi|13128997|ref|NM_024041.1|[13128997]; 2447: NM_024045,
“Homo sapiens DEAD (Asp-Glu-Ala-Asp) box polypeptide 50 (DDX50), mRNA”,
gi|13129005|ref|NM_024045.1|[13129005]; 2448: NM_024051, “Homo sapiens chromosome 7
open reading frame 24 (C7orf24), mRNA”, gi|34147353|ref|NM_024051.2|[34147353]; 2449:
NM_024052, “Homo sapiens hypothetical protein MGC3048 (MGC3048), mRNA”,
gi|23111006|ref|NM_024052.3|[23111006]; 2450: NM_024053, “Homo sapiens chromosome 22
open reading frame 18 (C22orf18), mRNA”, gi|37059723|ref|NM_024053.2|[37059723]; 2451:
NM_024057, “Homo sapiens nucleoporin Nup37 (Nup37), mRNA”,
gi|34222120|ref|NM_024057.2|[34222120]; 2452: NM_024065, “Homo sapiens phosducin-like
3 (PDCL3), mRNA”, gi|34147358|ref|NM_024065.2|[34147358]; 2453: NM_024068 “Homo
sapiens hypothetical protein MGC2731 (MGC2731), mRNA”,
gi|34147355|ref|NM_024068.2|[34147355]; 2454: NM_024072, “Homo sapiens DEAD (Asp-
Glu-Ala-Asp) box polypeptide 54 (DDX54), mRNA”,
gi|19923594|ref|NM_024072.2|[19923594]; 2455: NM_024075, “Homo sapiens leukocyte
receptor cluster (LRC) member 5 (LENG5), mRNA”,
gi|13129061|ref|NM_024075.1|[13129061]; 2456: NM_024076, “Homo sapiens potassium
channel tetramerisation domain containing 15 (KCTD15),”, mRNA,
gi|13129063|ref|NM_024076.1|[13129063]; 2457: NM_024078, “Homo sapiens hypothetical
protein MGC3162 (MGC3162), mRNA”, gi|13129067|ref|NM_024078.1|[13129067]; 2458:
NM_024080, “Homo sapiens transient receptor potential cation channel, subfamily M, member
8”, “(TRPM8), mRNA”, gi|21361690|ref|NM_024080.3|[21361690]; 2459: NM_024082,
“Homo sapiens transmembrane gamma-carboxyglutamic acid protein 3 (TMG3), mRNA”,
gi|31543810|ref|NM_024082.2|[31543810]; 2460: NM_024083, “Homo sapiens alveolar soft
part sarcoma chromosome region, candidate 1”, “(ASPSCR1), mRNA”,
gi|17572803|ref|NM_024083.2|[17572803]; 2461: NM_024089, “Homo sapiens KDEL (Lys-
Asp-Glu-Leu) containing 1 (KDELC1), mRNA”, gi|13129085|ref|NM_024089.1|[13129085];
2462: NM_024092, “Homo sapiens hypothetical protein MGC5508 (MGC5508), mRNA”,
gi|13129091|ref|NM_024092.1|[13129091]; 2463: NM_024093, “Homo sapiens hypothetical
protein MGC5509 (MGC5509), mRNA”, gi|13129093|ref|NM_024093.1|[13129093]; 2464:
NM_024094, Homo sapiens defective in sister chromatid cohesion homolog 1 (S. cerevisiae),
“(MGC5528), mRNA”, gi|13129095|ref|NM_024094.1|[13129095]; 2465: NM_024095, “Homo
sapiens ankyrin repeat and SOCS box-containing 8 (ASB8), mRNA”,
gi|40556379|ref|NM_024095.2|[40556379]; 2466: NM_024096, “Homo sapiens XTP3-
transactivated protein A (XTP3TPA), mRNA”, gi|13129099|ref|NM_024096.1|[13129099];
2467: NM_024107, “Homo sapiens hypothetical protein MGC3123 (MGC3123), mRNA”,
gi|13129117|ref|NM_024107.1|[13129117]; 2468: NM_024111, “Homo sapiens hypothetical
protein MGC4504 (MGC4504), mRNA”, gi|34147362|ref|NM_024111.2|[34147362]; 2469:
NM_024113, “Homo sapiens hypothetical protein MGC4707 (MGC4707), mRNA”,
gi|34147364|ref|NM_024113.2|[34147364]; 2470: NM_024115,, ref|NM_024115.1|[13129133],
This record was replaced or removed. See revision history for details.,, 2471: NM_024117,
“Homo sapiens mitogen-activated protein kinase associated protein 1 (MAPKAP1),”, mRNA,
gi|34147366|ref|NM_024117.2|[34147366]; 2472: NM_024119, “Homo sapiens likely ortholog
of mouse D11lgp2 (LGP2), mRNA”, gi|13129141|ref|NM_024119.1|[13129141]; 2473:
NM_024122, “Homo sapiens hypothetical protein MGC4825 (MGC4825), mRNA”,
gi|34147363|ref|NM_024122.2|[34147363]; 2474: NM_024292, “Homo sapiens ubiquitin-like 5
(UBL5), mRNA”, gi|42476283|ref|NM_024292.2|[42476283]; 2475: NM_024294, “Homo
sapiens hypothetical protein MGC4614 (MGC4614), mRNA”,
gi|13236513|ref|NM_024294.1|[13236513]; 2476: NM_024299, “Homo sapiens chromosome 20
open reading frame 149 (C20orf149), mRNA”, gi|34147371|ref|NM_024299.2|[34147371];
2477: NM_024300, “Homo sapiens coiled-coil-helix-coiled-coil-helix domain containing 7
(CHCHD7),”, mRNA, gi|34147367|ref|NM_024300.2|[34147367]; 2478: NM_024301, “Homo
sapiens fukutin related protein (FKRP), mRNA”, gi|36951139|ref|NM_024301.2|[36951139];
2479: NM_024302, “Homo sapiens matrix metalloproteinase 28 (MMP28), transcript variant 1,
mRNA”, gi|14589910|ref|NM_024302.2|[14589910]; 2480: NM_024311, “Homo sapiens
hypothetical protein ET (ET), mRNA”, gi|34147375|ref|NM_024311.2|[34147375]; 2481:
NM_024321, “Homo sapiens hypothetical protein MGC10433 (MGC10433), mRNA”,
gi|34147641|ref|NM_024321.3|[34147641]; 2482: NM_024322, “Homo sapiens hypothetical
protein MGC11266 (MGC11266), mRNA”, gi|13236564|ref|NM_024322.1|[13236564]; 2483:
NM_024323, “Homo sapiens hypothetical protein MGC11271 (MGC11271), mRNA”,
gi|31543147|ref|NM_024323.3|[31543147]; 2484: NM_024330, “Homo sapiens solute carrier
family 27 (fatty acid transporter), member 3”, “(SLC27A3), mRNA”,
gi|13236578|ref|NM_024330.1|[13236578]; 2485: NM_024331, “Homo sapiens chromosome 20
open reading frame 121 (C20orf121), mRNA”, gi|34147379|ref|NM_024331.2|[34147379];
2486: NM_024339, “Homo sapiens hypothetical protein MGC2655 (MGC2655), mRNA”,
gi|31543163|ref|NM_024339.2|[31543163]; 2487: NM_024409, “Homo sapiens natriuretic
peptide precursor C (NPPC), mRNA”, gi|13249345|ref|NM_024409.1|[13249345]; 2488:
NM_024411, “Homo sapiens prodynorphin (PDYN), mRNA”,
gi|32483402|ref|NM_024411.2|[32483402]; 2489: NM_024419, “Homo sapiens
phosphatidyiglycerophosphate synthase (PGS1), mRNA”,
gi|21314623|ref|NM_024419.2|[21314623]; 2490: NM_024491, “Homo sapiens p10-binding
protein (BITE), mRNA”, gi|13346499|ref|NM_024491.1|[13346499]; 2491: NM_024504,
“Homo sapiens PR domain containing 14 (PRDM14), mRNA”,
gi|41349468|ref|NM_024504.2|[41349468]; 2492: NM_024505, “Homo sapiens NADPH
oxidase, EF hand calcium-binding domain 5 (NOX5), mRNA”,
gi|20127623|ref|NM_024505.2|[20127623]; 2493: NM_024506, “Homo sapiens galactosidase,
beta 1-like (GLB1L), mRNA”, gi|40255042|ref|NM_024506.3|[40255042]; 2494: NM_024507,
“Homo sapiens kringle containing transmembrane protein 2 (KREMEN2), transcript”, “variant 2,
mRNA”, gi|27437002|ref|NM_024507.2|[27437002]; 2495: NM_024512, “Homo sapiens
leucine rich repeat containing 2 (LRRC2), mRNA”, gi|14719432|ref|NM_024512.2|[14719432];
2496: NM_024523, “Homo sapiens GRIP and coiled-coil domain-containing 1 (GCC1),
mRNA”, gi|34305454|ref|NM_024523.5|[34305454]; 2497: NM_024525, “Homo sapiens
tetratricopeptide repeat domain 13 (TTC13), mRNA”,
gi|31377702|ref|NM_024525.2|[31377702]; 2498: NM_024526, “Homo sapiens EPS8-like 3
(EPS8L3), transcript variant 3, mRNA”, gi|21071013|ref|NM_024526.2|[21071013]; 2499:
NM_024536, “Homo sapiens chondroitin polymerizing factor (CHPF), mRNA”,
gi|34222219|ref|NM_024536.4|[34222219]; 2500: NM_024537, “Homo sapiens hypothetical
protein FLJ12118 (FLJ12118), mRNA”, gi|13375694|ref|NM_024537.1|[13375694]; 2501:
NM_024540, “Homo sapiens mitochondrial ribosomal protein L24 (MRPL24), nuclear gene
encoding”, “mitochondrial protein, transcript variant 2, mRNA”,
gi|22035587|ref|NM_024540.2|[22035587]; 2502: NM_024544, “Homo sapiens hypothetical
protein FLJ12875 (FLJ12875), mRNA”, gi|13375704|ref|NM_024544.1|[13375704]; 2503:
NM_024546, “Homo sapiens chromosome 13 open reading frame 7 (C13orf7), mRNA”,
gi|21362045|ref|NM_024546.2|[21362045]; 2504: NM_024551, “Homo sapiens adiponectin
receptor 2 (ADIPOR2), mRNA”, gi|38261972|ref|NM_024551.2|[38261972]; 2505: NM_024554,
“Homo sapiens piggyBac transposable element derived 5 (PGBD5), mRNA”,
gi|25777747|ref|NM_024554.2|[25777747]; 2506: NM_024565, “Homo sapiens hypothetical
protein FLJ14166 (FLJ14166), mRNA”, gi|40018623|ref|NM_024565.4|[40018623]; 2507:
NM_024570, “Homo sapiens hypothetical protein FLJ11712 (FLJ11712), mRNA”,
gi|13375741|ref|NM_024570.1|[13375741]; 2508: NM_024572, Homo sapiens UDP-N-acetyl-
alpha-D-galactosamine:polypeptide, “N-acetylgalactosaminyltransferase 14 (GalNAc-T14)
(GALNT14), mRNA”, gi|13375743|ref|NM_024572.1|[13375743]; 2509: NM_024573, “Homo
sapiens chromosome 6 open reading frame 211 (C6orf211), mRNA”,
gi|13375745|ref|NM_024573.1|[13375745]; 2510: NM_024580, “Homo sapiens hypothetical
protein FLJ13119 (FLJ13119), mRNA”, gi|40255246|ref|NM_024580.3|[40255246]; 2511:
NM_024583, “Homo sapiens secernin 3 (SCRN3), mRNA”,
gi|38504670|ref|NM_024583.2|[38504670]; 2512: NM_024584, “Homo sapiens hypothetical
protein FLJ13646 (FLJ13646), mRNA”, gi|39979625|ref|NM_024584.2|[39979625]; 2513:
NM_024585, “Homo sapiens hypothetical protein FLJ22160 (FLJ22160), mRNA”,
gi|20149678|ref|NM_024585.2|[20149678]; 2514: NM_024587, “Homo sapiens hypothetical
protein FLJ22353 (FLJ22353), mRNA”, gi|42734433|ref|NM_024587.2|[42734433]; 2515:
NM_024589, “Homo sapiens leucine zipper domain protein (FLJ22386), mRNA”,
gi|13375778|ref|NM_024589.1|[13375778]; 2516: NM_024590, “Homo sapiens hypothetical
protein FLJ23548 (FLJ23548), mRNA”, gi|40254961|ref|NM_024590.2|[40254961]; 2517:
NM_024594, “Homo sapiens pantothenate kinase 3 (PANK3), mRNA”,
gi|24430178|ref|NM_024594.2|[24430178]; 2518: NM_024595, “Homo sapiens hypothetical
protein FLJ12666 (FLJ12666), mRNA”, gi|13375790|ref|NM_024595.1|[13375790]; 2519:
NM_024598, “Homo sapiens hypothetical protein FLJ13154 (FLJ13154), mRNA”,
gi|42716282|ref|NM_024598.2|[42716282]; 2520: NM_024599, “Homo sapiens hypothetical
protein FLJ22341 (FLJ22341), mRNA”, gi|24432005|ref|NM_024599.2|[24432005]; 2521:
NM_024600, “Homo sapiens hypothetical protein FLJ20898 (FLJ20898), mRNA”,
gi|13375800|ref|NM_024600.1|[13375800]; 2522: NM_024604, “Homo sapiens hypothetical
protein FLJ21908 (FLJ21908), mRNA”, gi|13375808|ref|NM_024604.1|[13375808]; 2523:
NM_024608, “Homo sapiens nei endonuclease VIII-like 1 (E. coli) (NEIL1), mRNA”,
gi|13375816|ref|NM_024608.1|[13375816]; 2524: NM_024609,, ref|NM_024609.1|[13375818],
This record was temporarily removed by RefSeq staff for additional review.,, 2525:
NM_024611, “Homo sapiens NMDA receptor-regulated gene 2 (NARG2), mRNA”,
gi|37202122|ref|NM_024611.2|[37202122]; 2526: NM_024615, “Homo sapiens hypothetical
protein FLJ21308 (FLJ21308), mRNA”, gi|24432008|ref|NM_024615.2|[24432008]; 2527:
NM_024616, “Homo sapiens hypothetical protein FLJ23186 (FLJ23186), mRNA”,
gi|13375833|ref|NM_024616.1|[13375833]; 2528: NM_024618, “Homo sapiens NOD9 protein
(NOD9), transcript variant 1, mRNA”, gi|25777607|ref|NM_024618.2|[25777607]; 2529:
NM_024624, Homo sapiens SMC6 structural maintenance of chromosomes 6-like 1 (yeast),
“(SMC6L1), mRNA”, gi|31543646|ref|NM_024624.2|[31543646]; 2530: NM_024628, “Homo
sapiens solute carrier family 12 (potassium/chloride transporters), member”, “8 (SLC12A8),
mRNA”, gi|42740889|ref|NM_024628.4|[42740889]; 2531: NM_024630, “Homo sapiens zinc
finger, DHHC domain containing 14 (ZDHHC14), mRNA”,
gi|24371240|ref|NM_024630.2|[24371240]; 2532: NM_024631, “Homo sapiens hypothetical
protein FLJ23342 (FLJ23342), mRNA”, gi|13375859|ref|NM_024631.1|[13375859]; 2533:
NM_024643, “Homo sapiens chromosome 14 open reading frame 140 (C14orf140), mRNA”,
gi|13375882|ref|NM_024643.1|[13375882]; 2534: NM_024650, “Homo sapiens hypothetical
protein FLJ22531 (FLJ22531), mRNA”, gi|31542734|ref|NM_024650.2|[31542734]; 2535:
NM_024654, “Homo sapiens hypothetical protein FLJ23323 (FLJ23323), mRNA”,
gi|40217804|ref|NM_024654.3|[40217804]; 2536: NM_024658, “Homo sapiens importin 4
(IPO4), mRNA”, gi|18874098|ref|NM_024658.2|[18874098]; 2537: NM_024659, “Homo
sapiens hypothetical protein FLJ11753 (FLJ11753), mRNA”,
gi|40254964|ref|NM_024659.2|[40254964]; 2538: NM_024660, “Homo sapiens hypothetical
protein FLJ22573 (FLJ22573), mRNA”, gi|13375912|ref|NM_024660.1|[13375912]; 2539:
NM_024667, “Homo sapiens hypothetical protein FLJ12750 (FLJ12750), mRNA”,
gi|13375925|ref|NM_024667.1|[13375925]; 2540: NM_024669, “Homo sapiens hypothetical
protein FLJ11795 (FLJ11795), mRNA”, gi|13375927|ref|NM_024669.1|[13375927]; 2541:
NM_024670, “Homo sapiens suppressor of variegation 3-9 homolog 2 (Drosophila)
(SUV39H2),”, mRNA, gi|34147611|ref|NM_024670.3|[34147611]; 2542: NM_024672, “Homo
sapiens THAP domain containing 9 (THAP9), mRNA”,
gi|38564326|ref|NM_024672.2|[38564326]; 2543: NM_024674, “Homo sapiens lin-28 homolog
(C. elegans) (LIN28), mRNA”, gi|34222338|ref|NM_024674.3|[34222338]; 2544: NM_024675,
“Homo sapiens hypothetical protein FLJ21816 (FLJ21816), mRNA”,
gi|27436909|ref|NM_024675.2|[27436909]; 2545: NM_024678, “Homo sapiens hypothetical
protein FLJ23441 (FLJ23441), mRNA”, gi|39725682|ref|NM_024678.3|[39725682]; 2546:
NM_024682, “Homo sapiens TBC1 domain family, member 17 (TBC1D17), mRNA”,
gi|13375951|ref|NM_024682.1|[13375951]; 2547: NM_024683, “Homo sapiens hypothetical
protein FLJ22729 (FLJ22729), mRNA”, gi|13375953|ref|NM_024683.1|[13375953]; 2548:
NM_024685, “Homo sapiens hypothetical protein ELJ23560 (FLJ23560), mRNA”,
gi|31377692|ref|NM_024685.2|[31377692]; 2549: NM_024696, “Homo sapiens hypothetical
protein FLJ23058 (FLJ23058), mRNA”, gi|13375978|ref|NM_024696.1|[13375978]; 2550:
NM_024698, “Homo sapiens solute carrier family 25 (mitochondrial carrier: glutamate),
member”, “22 (SLC25A22), mRNA”, gi|34222352|ref|NM_024698.4|[34222352]; 2551:
NM_024699, “Homo sapiens hypothetical protein FLJ14007 (FLJ14007), mRNA”,
gi|13375984|ref|NM_024699.1|[13375984]; 2552: NM_024700, “Homo sapiens Smad nuclear
interacting protein (SNIP1), mRNA”, gi|21314719|ref|NM_024700.2|[21314719]; 2553:
NM_024703, “Homo sapiens hypothetical protein FLJ22593 (FLJ22593), mRNA”,
gi|31542737|ref|NM_024703.2|[31542737]; 2554: NM_024706, “Homo sapiens hypothetical
protein FLJ13479 (FLJ13479), mRNA”, gi|39725704|ref|NM_024706.3|[39725704]; 2555:
NM_024708, “Homo sapiens ankyrin repeat and SOCS box-containing 7 (ASB7), transcript
variant”, “1, mRNA”, gi|30089993|ref|NM_024708.2|[30089993]; 2556: NM_024711, “Homo
sapiens human immune associated nucleotide 2 (hIAN2), mRNA”,
gi|28416428|ref|NM_024711.2|[28416428]; 2557: NM_024712, “Homo sapiens engulfment and
cell motility 3 (ced-12 homolog, C. elegans)”, “(ELMO3), mRNA”,
gi|19718770|ref|NM_024712.2|[19718770]; 2558: NM_024718, “Homo sapiens FLJ10101
protein (FLJ10101), mRNA”, gi|38201703|ref|NM_024718.2|[38201703]; 2559: NM_024723,
“Homo sapiens MICAL-like 2 (FLJ23471), transcript variant 2, mRNA”,
gi|13376030|ref|NM_024723.1|[13376030]; 2560: NM_024728, “Homo sapiens chromosome 7
open reading frame 10 (C7orf10), mRNA”, gi|13376041|ref|NM_024728.1|[13376041]; 2561:
NM_024731, “Homo sapiens chromosome 16 open reading frame 44 (C16orf44), mRNA”,
gi|31542245|ref|NM_024731.2|[31542245]; 2562: NM_024741, “Homo sapiens zinc finger
protein 408 (ZNF408), mRNA”, gi|13376063|ref|NM_024741.1|[13376063]; 2563: NM_024744,
“Homo sapiens amyotrophic lateral sclerosis 2 (juvenile) chromosome region,”, “candidate 8
(ALS2CR8), mRNA”, gi|20806094|ref|NM_024744.12|[20806094]; 2564: NM_024745, “Homo
sapiens likely ortholog of mouse Shc SH2-domain binding protein 1 (SHCBP1),”, mRNA,
gi|24850112|ref|NM_024745.2|[24850112]; 2565: NM_024747, “Homo sapiens Hermansky-
Pudlak syndrome 6 (HPS6), mRNA”, gi|31881784|ref|NM_024747.4|[31881784]; 2566:
NM_024756, “Homo sapiens elastin microfibril interfacer 3 (EMILIN3), mRNA”,
gi|13376090|ref|NM_024756.1|[13376090]; 2567: NM_024760, “Homo sapiens transducin-like
enhancer protein 6 (FLJ14009), mRNA”, gi|13376098|ref|NM_024760.1|[13376098]; 2568:
NM_024761, “Homo sapiens MOB1, Mps One Binder kinase activator-like 2B (yeast)
(MOBKL2B),”, mRNA, gi|41350329|ref|NM_024761.3|[41350329]; 2569: NM_024763,
“Homo sapiens hypothetical protein FLJ23129 (FLJ23129), mRNA”,
gi|33946333|ref|NM_024763.2|[33946333]; 2570: NM_024770, “Homo sapiens hypothetical
protein FLJ13984 (FLJ13984), mRNA”, gi|13376116|ref|NM_024770.1|[13376116]; 2571:
NM_024771, “Homo sapiens hypothetical protein FLJ13848 (FLJ13848), mRNA”,
gi|13376118|ref|NM_024771.1|[13376118]; 2572: NM_024778, “Homo sapiens ring finger
protein 127 (RNF127), mRNA”, gi|37622895|ref|NM_024778.3|[37622895]; 2573: NM_024779,
“Homo sapiens phosphatidylinositol-4-phosphate 5-kinase, type II, gamma”, “(PIP5K2C),
mRNA”, gi|37059743|ref|NM_024779.3|[37059743]; 2574: NM_024782, “Homo sapiens
hypothetical protein FLJ12610 (FLJ12610), mRNA”,
gi|13376141|ref|NM_024782.1|[13376141]; 2575: NM_024783, “Homo sapiens hypothetical
protein FLJ23598 (FLJ23598), mRNA”, gi|31657118|ref|NM_024783.2|[31657118]; 2576:
NM_024785, “Homo sapiens hypothetical protein FLJ22746 (FLJ22746), mRNA”,
gi|31542740|ref|NM_024785.2|[31542740]; 2577: NM_024799, “Homo sapiens hypothetical
protein FLJ13224 (FLJ13224), mRNA”, gi|13376172|ref|NM_024799.1|[13376172]; 2578:
NM_024800, “Homo sapiens NIMA (never in mitosis gene a)-related kinase 11 (NEK11),
mRNA”, gi|22094142|ref|NM_024800.2|[22094142]; 2579: NM_024806, “Homo sapiens
hypothetical protein FLJ23554 (FLJ23554), transcript variant 1,”, mRNA,
gi|40217798|ref|NM_024806.2|[40217798]; 2580: NM_024808, “Homo sapiens FLJ22624
protein (FLJ22624), mRNA”, gi|38505206|ref|NM_024808.2|[38505206]; 2581: NM_024811,
“Homo sapiens pre-mRNA cleavage factor I, 59 kDa subunit (FLJ12529), mRNA”,
gi|24432015|ref|NM_024811.2|[24432015]; 2582: NM_024818, “Homo sapiens ubiquitin-
activating enzyme E1-domain containing 1 (UBE1DC1),”, “transcript variant 1, mRNA”,
gi|38327030|ref|NM_024818.2|[38327030]; 2583: NM_024821, “Homo sapiens hypothetical
protein FLJ22349 (FLJ22349), mRNA”, gi|13376215|ref|NM_024821.1|[13376215]; 2584:
NM_024823,, ref|NM_024823.1|[13376219], This record was temporarily removed by RefSeq
staff for additional review.,, 2585: NM_024827, “Homo sapiens histone deacetylase 11
(HDAC11), mRNA”, gi|13376227|ref|NM_024827.1|[13376227]; 2586: NM_024828, “Homo
sapiens chromosome 9 open reading frame 82 (C9orf82), mRNA”,
gi|13376229|ref|NM_024828.1|[13376229]; 2587: NM_024831, “Homo sapiens nuclear receptor
coactivator 6 interacting protein (NCOA6IP), mRNA”,
gi|19923660|ref|NM_024831.5|[19923660]; 2588: NM_024834, “Homo sapiens hypothetical
protein FLJ13081 (FLJ13081), mRNA”, gi|13376242|ref|NM_024834.1|[13376242]; 2589:
NM_024848, “Homo sapiens hypothetical protein FLJ13941 (FLJ13941), mRNA”,
gi|13376266|ref|NM_024848.1|[13376266]; 2590: NM_024849, “Homo sapiens hypothetical
protein FLJ14126 (FLJ14126), mRNA”, gi|13376268|ref|NM_024849.1|[13376268]; 2591:
NM_024852, “Homo sapiens eukaryotic translation initiation factor 2C, 3 (EIF2C3), transcript”,
“variant 1, mRNA”, gi|29294646|ref|NM_024852.2|[29294646]; 2592: NM_024853, “Homo
sapiens hypothetical protein FLJ13385 (FLJ13385), mRNA”,
gi|13376276|ref|NM_024853.1|[13376276]; 2593: NM_024860, “Homo sapiens hypothetical
protein FLJ21148 (FLJ21148), mRNA”, gi|13376287|ref|NM_024860.1|[13376287]; 2594:
NM_024861, “Homo sapiens hypothetical protein FLJ22671 (FLJ22671), mRNA”,
gi|13376289|ref|NM_024861.1|[13376289]; 2595: NM_024869, “Homo sapiens hypothetical
protein FLJ14050 (FLJ14050), mRNA”, gi|13376303|ref|NM_024869.1|[13376303]; 2596:
NM_024871, “Homo sapiens hypothetical protein FLJ12748 (FLJ12748), mRNA”,
gi|13376305|ref|NM_024871.1|[13376305]; 2597: NM_024884, “Homo sapiens chromosome 14
open reading frame 160 (C14orf160), mRNA”, gi|13376330|ref|NM_024884.1|[13376330];
2598: NM_024887, “Homo sapiens dehydrodolichyl diphosphate synthase (DHDDS), mRNA”,
gi|13376336|ref|NM_024887.1|[13376336]; 2599: NM_024888, “Homo sapiens hypothetical
protein FLJ11535 (FLJ11535), mRNA”, gi|13376338|ref|NM_024888.1|[13376338]; 2600:
NM_024894, “Homo sapiens hypothetical protein FLJ14075 (FLJ14075), mRNA”,
gi|13430871|ref|NM_024894.1|[13430871]; 2601: NM_024900, “Homo sapiens PHD protein
Jade-1 (JADE1), transcript variant S, mRNA”, gi|19923608|ref|NM_024900.2|[19923608]; 2602:
NM_024901, “Homo sapiens hypothetical protein FLJ22457 (FLJ22457), mRNA”,
gi|34147689|ref|NM_024901.3|[34147689]; 2603: NM_024902, “Homo sapiens hypothetical
protein FLJ13236 (FLJ13236), mRNA”, gi|24431938|ref|NM_024902.2|[24431938]; 2604:
NM_024906, “Homo sapiens stearoyl-CoA desaturase 4 (SCD4), mRNA”,
gi|13376362|ref|NM_024906.1|[13376362]; 2605: NM_024908, “Homo sapiens hypothetical
protein FLJ12973 (FLJ12973), mRNA”, gi|13376366|ref|NM_024908.1|[13376366]; 2606:
NM_024909, “Homo sapiens chromosome 6 open reading frame 134 (C6orf134), mRNA”,
gi|13376368|ref|NM_024909.1|[13376368]; 2607: NM_024911, “Homo sapiens putative NFkB
activating protein 373 (FLJ23091), mRNA”, gi|31542744|ref|NM_024911.3|[31542744]; 2608:
NM_024913, “Homo sapiens hypothetical protein FLJ21986 (FLJ21986), mRNA”,
gi|31542726|ref|NM_024913.3|[31542726]; 2609: NM_024927, “Homo sapiens hypothetical
protein FLJ21019 (FLJ21019), mRNA”, gi|40255046|ref|NM_024927.3|[40255046]; 2610:
NM_024928, “Homo sapiens hypothetical protein FLJ22559 (FLJ22559), mRNA”,
gi|34147613|ref|NM_024928.3|[34147613]; 2611: NM_024935, “Homo sapiens KIAA1772
(KIAA1772), mRNA”, gi|40354206|ref|NM_024935.2|[40354206]; 2612: NM_024939, “Homo
sapiens hypothetical protein FLJ21918 (FLJ21918), mRNA”,
gi|13435148|ref|NM_024939.1|[13435148]; 2613: NM_024941, “Homo sapiens hypothetical
protein FLJ13611 (FLJ13611), mRNA”, gi|13376418|ref|NM_024941.1|[13376418]; 2614:
NM_024946, “Homo sapiens NEFA-interacting nuclear protein NIP30 (NIP30), mRNA”,
gi|13376428|ref|NM_024946.1|[13376428]; 2615: NM_024948, “Homo sapiens hypothetical
protein FLJ13397 (FLJ13397), mRNA”, gi|13376430|ref|NM_024948.1|[13376430]; 2616:
NM_024954, “Homo sapiens hypothetical protein FLJ11807 (FLJ11807), mRNA”,
gi|34222339|ref|NM_024954.3|[34222339]; 2617: NM_024955, “Homo sapiens hypothetical
protein FLJ23322 (FLJ23322), mRNA”, gi|34303916|ref|NM_024955.4|[34303916]; 2618:
NM_024956, “Homo sapiens hypothetical protein FLJ23375 (FLJ23375), mRNA”,
gi|20070341|ref|NM_024956.2|[20070341]; 2619: NM_024958, “Homo sapiens chromosome 20
open reading frame 98 (C20orf98), mRNA”, gi|13376446|ref|NM_024958.1|[13376446]; 2620:
NM_024959, “Homo sapiens solute carrier family 24 (sodium/potassium/calcium exchanger),”,
“member 6 (NCKX6), mRNA”, gi|39995085|ref|NM_024959.2|[39995085]; 2621: NM_024960,
“Homo sapiens pantothenate kinase 2 (Hallervorden-Spatz syndrome) (PANK2),”, “transcript
variant 5, mRNA”, gi|24430166|ref|NM_024960.3|[24430166]; 2622: NM_024988, “Homo
sapiens hypothetical protein FLJ12355 (FLJ12355), mRNA”,
gi|13376491|ref|NM_024988.1|[13376491]; 2623: NM_024989, “Homo sapiens GPI deacylase
(PGAP1), mRNA”, gi|13376493|ref|NM_024989.1|[13376493]; 2624: NM_024996, “Homo
sapiens mitochondrial elongation factor G1 (EFG1), nuclear gene encoding”, “mitochondrial
protein, mRNA”, gi|25306276|ref|NM_024996.5|[25306276]; 2625: NM_025000, “Homo
sapiens hypothetical protein FLJ13096 (FLJ13096), mRNA”,
gi|13376510|ref|NM_025000.1|[13376510]; 2626: NM_025003, Homo sapiens a disintegrin-like
and metalloprotease (reprolysin type) with, “thrombospondin type 1 motif, 20 (ADAMTS20),
transcript variant 1, mRNA”, gi|28460689|ref|NM_025003.2|[28460689]; 2627: NM_025009,
“Homo sapiens hypothetical protein FLJ13621 (FLJ13621), mRNA”,
gi|13376528|ref|NM_025009.1|[13376528]; 2628: NM_025029, “Homo sapiens hypothetical
protein FLJ14346 (FLJ14346), mRNA”, gi|13376551|ref|NM_025029.1|[13376551]; 2629:
NM_025034, “Homo sapiens hypothetical protein FLJ21290 (FLJ21290), mRNA”,
gi|13376561|ref|NM_025034.1|[13376561]; 2630: NM_025045, “Homo sapiens hypothetical
protein FLJ22582 (FLJ22582), mRNA”, gi|34147690|ref|NM_025045.3|[34147690]; 2631:
NM_025054, Homo sapiens valosin-containing protein (p97)/p47 complex-interacting protein,
“p135 (VCIP135), mRNA”, gi|38569451|ref|NM_025054.3|[38569451]; 2632: NM_025058,
“Homo sapiens tripartite motif-containing 46 (TRIM46), mRNA”,
gi|42415489|ref|NM_025058.2|[42415489]; 2633: NM_025061, “Homo sapiens hypothetical
protein FLJ23420 (FLJ23420), mRNA”, gi|40217802|ref|NM_025061.3|[40217802]; 2634:
NM_025064, “Homo sapiens hypothetical protein FLJ23604 (FLJ23604), mRNA”,
gi|13376602|ref|NM_025064.1|[13376602]; 2635: NM_025065, “Homo sapiens RNA
processing factor 1 (RPF1), mRNA”, gi|38569467|ref|NM_025065.5|[38569467]; 2636:
NM_025072, “Homo sapiens prostaglandin E synthase 2 (PTGES2), transcript variant 1,
mRNA”, gi|40068467|ref|NM_025072.4|[40068467]; 2637: NM_025074, “Homo sapiens Fraser
syndrome 1 (FRAS1), mRNA”, gi|33413413|ref|NM_025074.2|[33413413]; 2638: NM_025076,
“Homo sapiens UDP-glucuronate decarboxylase 1 (UXS1), mRNA”,
gi|42516562|ref|NM_025076.2|[42516562]; 2639: NM_025079, “Homo sapiens hypothetical
protein FLJ23231 (FLJ23231), mRNA”, gi|13376631|ref|NM_025079.1|[13376631]; 2640:
NM_025083, “Homo sapiens hypothetical protein FLJ21128 (FLJ21128), mRNA”,
gi|19923612|ref|NM_025083.2|[19923612]; 2641: NM_025090, “Homo sapiens ubiquitin
specific protease 36 (USP36), mRNA”, gi|35250685|ref|NM_025090.2|[35250685]; 2642:
NM_025092, “Homo sapiens hypothetical protein FLJ22635 (FLJ22635), mRNA”,
gi|13376651|ref|NM_025092.1|[13376651]; 2643: NM_025097, “Homo sapiens hypothetical
protein FLJ21106 (FLJ21106), mRNA”, gi|13376659|ref|NM_025097.1|[13376659]; 2644:
NM_025106, “Homo sapiens SPRY domain-containing SOCS box protein SSB-1 (SSB1),
mRNA”, gi|18141315|ref|NM_025106.2|[18141315]; 2645: NM_025108, “Homo sapiens
hypothetical protein FLJ13909 (FLJ13909), mRNA”,
gi|13376678|ref|NM_025108.1|[13376678]; 2646: NM_025115, “Homo sapiens hypothetical
protein FLJ23263 (FLJ23263), mRNA”, gi|13376690|ref|NM_025115.1|[13376690]; 2647:
NM_025126, “Homo sapiens ring finger protein 34 (RNF34), transcript variant 2, mRNA”,
gi|37595536|ref|NM_025126.2|[37595536]; 2648: NM_025128, “Homo sapiens MUS81
endonuclease homolog (yeast) (MUS81), mRNA”, gi|34147593|ref|NM_025128.3|[34147593];
2649: NM_025129, “Homo sapiens hypothetical protein FLJ22688 (FLJ22688), mRNA”,
gi|34147614|ref|NM_025129.3|[34147614]; 2650: NM_025137, “Homo sapiens hypothetical
protein FLJ21439 (FLJ21439), mRNA”, gi|33636747|ref|NM_025137.2|[33636747]; 2651:
NM_025138, “Homo sapiens hypothetical protein FLJ12661 (FLJ12661), transcript variant 1,”,
mRNA, gi|25777603|ref|NM_025138.2|[25777603]; 2652: NM_025140, “Homo sapiens limkain
beta 2 (FLJ22471), mRNA”, gi|13376724|ref|NM_025140.1|[13376724]; 2653: NM_025141,
“Homo sapiens BBP-like protein 2 (BLP2), transcript variant 2, mRNA”,
gi|17865798|ref|NM_025141.2|[17865798]; 2654: NM_025147, “Homo sapiens hypothetical
protein FLJ13448 (FLJ13448), mRNA”, gi|31542687|ref|NM_025147.2|[31542687]; 2655:
NM_025150, “Homo sapiens threonyl-tRNA synthetase (FLJ12528), mRNA”,
gi|39725684|ref|NM_025150.3|[39725684]; 2656: NM_025155, “Homo sapiens hypothetical
protein FLJ11848 (FLJ11848), mRNA”, gi|13376750|ref|NM_025155.1|[13376750]; 2657:
NM_025181, “Homo sapiens solute carrier family 35, member F5 (SLC35F5), mRNA”,
gi|21361958|ref|NM_025181.2|[21361958]; 2658: NM_025201, “Homo sapiens PH domain-
containing protein (pp9099), mRNA”, gi|33457315|ref|NM_025201.3|[33457315]; 2659:
NM_025203, “Homo sapiens hypothetical protein FLJ21945 (FLJ21945), mRNA”,
gi|13376797|ref|NM_025203.1|[13376797]; 2660: NM_025212, “Homo sapiens CXXC finger 4
(CXXC4), mRNA”, gi|13376815|ref|NM_025212.1|[13376815]; 2661: NM_025215, “Homo
sapiens pseudouridylate synthase 1 (PUS1), mRNA”,
gi|34147590|ref|NM_025215.3|[34147590]; 2662: NM_025233, Homo sapiens bifunctional
phosphopantetheine adenylyl transferase/dephospho CoA, “kinase (DPCK), mRNA”,
gi|22095394|ref|NM_025233.3|[22095394]; 2663: NM_025235, “Homo sapiens tankyrase,
TRF1-interacting ankyrin-related ADP-ribose polymerase 2”, “(TNKS2), mRNA”,
gi|21361945|ref|NM_025235.2|[21361945]; 2664: NM_025236, “Homo sapiens ring finger
protein 39 (RNF39), transcript variant 1, mRNA”, gi|25777714|ref|NM_025236.2|[25777714];
2665: NM_025241, “Homo sapiens UBX domain containing 1 (UBXD1), mRNA”,
gi|13376853|ref|NM_025241.1|[13376853]; 2666: NM_025243, “Homo sapiens solute carrier
family 19, member 3 (SLC19A3), mRNA”, gi|21361938|ref|NM_025243.2|[21361938]; 2667:
NM_025247, “Homo sapiens hypothetical protein MGC5601 (MGC5601), mRNA”,
gi|31543200|ref|NM_025247.2|[31543200]; 2668: NM_025260, “Homo sapiens chromosome 6
open reading frame 25 (C6orf25), transcript variant 1,”, mRNA,
gi|19913372|ref|NM_025260.2|[19913372]; 2669: NM_025263, “Homo sapiens proline-rich
polypeptide 3 (PRR3), mRNA”, gi|13376877|ref|NM_025263.1|[13376877]; 2670: NM_025267,
“Homo sapiens hypothetical protein MGC2744 (MGC2744), mRNA”,
gi|34147388|ref|NM_025267.2|[34147388]; 2671: NM_030567, “Homo sapiens hypothetical
protein MGC10772 (MGC10772), mRNA”, gi|21361936|ref|NM_030567.2|[21361936]; 2672:
NM_030576, “Homo sapiens hypothetical protein MGC10986 (MGC10986), mRNA”,
gi|22095372|ref|NM_030576.2|[22095372]; 2673: NM_030577, “Homo sapiens hypothetical
protein MGC10993 (MGC10993), mRNA”, gi|13386491|ref|NM_030577.1|[13386491]; 2674:
NM_030664, “Homo sapiens phosphotriesterase related (PTER), mRNA”,
gi|20070185|ref|NM_030664.2|[20070185]; 2675: NM_030673, “Homo sapiens SEC13-like 1
(S. cerevisiae) (SEC13L1), transcript variant 1, mRNA”,
gi|34335135|ref|NM_030673.2|[34335135]; 2676: NM_030674, “Homo sapiens solute carrier
family 38, member 1 (SLC38A1), mRNA”, gi|21361928|ref|NM_030674.2|[21361928]; 2677:
NM_030758, “Homo sapiens oxysterol binding protein 2 (OSBP2), mRNA”,
gi|13540512|ref|NM_030758.1|[13540512]; 2678: NM_030761, “Homo sapiens wingless-type
MMTV integration site family, member 4 (WNT4), mRNA”,
gi|17402921|ref|NM_030761.2|[17402921]; 2679: NM_030762, “Homo sapiens basic helix-
loop-helix domain containing, class B, 3 (BHLHB3), mRNA”,
gi|13540520|ref|NM_030762.1|[13540520]; 2680: NM_030780, “Homo sapiens mitochondrial
folate transporter/carrier (MFTC), mRNA”, gi|21314738|ref|NM_030780.2|[21314738]; 2681:
NM_030784, “Homo sapiens G protein-coupled receptor 63 (GPR63), mRNA”,
gi|13540556|ref|NM_030784.1|[13540556]; 2682: NM_030790, “Homo sapiens T-cell
immunomodulatory protein (CDA08), mRNA”, gi|21361932|ref|NM_030790.2|[21361932];
2683: NM_030791, “Homo sapiens sphingosine-1-phosphate phosphatase 1 (SGPP1), mRNA”,
gi|40254975|ref|NM_030791.2|[40254975]; 2684: NM_030798, “Homo sapiens Williams-
Beuren syndrome chromosome region 16 (WBSCR16), transcript”, “variant 1, mRNA”,
gi|22538491|ref|NM_030798.2|[22538491]; 2685: NM_030804,, ref|NM_030804.1|[13540591],
This record was temporarily removed by RefSeq staff for additional review.,, 2686:
NM_030805, “Homo sapiens lectin, mannose-binding 2-like (LMAN2L), mRNA”,
gi|13540593|ref|NM_030805.1|[13540593]; 2687: NM_030806, “Homo sapiens chromosome 1
open reading frame 21 (C1orf21), mRNA”, gi|40788019|ref|NM_030806.2|[40788019]; 2688:
NM_030808, Homo sapiens nudE nuclear distribution gene E homolog like 1 (A. nidulans),
“(NDEL1), mRNA”, gi|31543284|ref|NM_030808.2|[31543284]; 2689: NM_030809, “Homo
sapiens chromosome 12 open reading frame 22 (C12orf22), mRNA”,
gi|13540601|ref|NM_030809.1|[13540601]; 2690: NM_030818, “Homo sapiens hypothetical
protein MGC10471 (MGC10471), mRNA”, gi|34147391|ref|NM_030818.2|[34147391]; 2691:
NM_030824, “Homo sapiens zinc finger protein 442 (ZNF442), mRNA”,
gi|13540500|ref|NM_030824.1|[13540500]; 2692: NM_030877, “Homo sapiens catenin, beta
like 1 (CTNNBL1), mRNA”, gi|29570786|ref|NM_030877.3|[29570786]; 2693: NM_030907,
“Homo sapiens hypothetical protein MGC10731 (MGC10731), mRNA”,
gi|34147392|ref|NM_030907.2|[34147392]; 2694: NM_030917, “Homo sapiens FIP1 like 1 (S. cerevisiae)
(FIP1L1), mRNA”, gi|40254977|ref|NM_030917.2|[40254977]; 2695: NM_030926,
“Homo sapiens integral membrane protein 2C (ITM2C), mRNA”,
gi|31560867|ref|NM_030926.3|[31560867]; 2696: NM_030927, “Homo sapiens tetraspanin
similar to TM4SF9 (DC-TM4F2), mRNA”, gi|13569888|ref|NM_030927.1|[13569888]; 2697:
NM_030954, “Homo sapiens hypothetical protein DKFZp564A022 (DKFZP564A022),
mRNA”, gi|21361953|ref|NM_030954.2|[21361953]; 2698: NM_030963, “Homo sapiens ring
finger protein 146 (RNF146), mRNA”, gi|33636757|ref|NM_030963.2|[33636757]; 2699:
NM_030968, “Homo sapiens C1q and tumor necrosis factor related protein 1 (C1QTNF1),”,
“transcript variant 1, mRNA”, gi|38372915|ref|NM_030968.2|[38372915]; 2700: NM_030974,
“Homo sapiens hypothetical protein DKFZp434N1923 (DKFZP434N1923), mRNA”,
gi|31542518|ref|NM_030974.2|[31542518]; 2701: NM_030978, “Homo sapiens actin related
protein 2/3 complex, subunit 5-like (ARPC5L), mRNA”,
gi|13569955|ref|NM_030978.1|[13569955]; 2702: NM_031210, “Homo sapiens hypothetical
protein DC50 (DC50), mRNA”, gi|33667026|ref|NM_031210.3|[33667026]; 2703: NM_031213,
“Homo sapiens hypothetical protein MGC5244 (MGG5244), mRNA”,
gi|21361948|ref|NM_031213.2|[21361948]; 2704: NM_031217, “Homo sapiens kinesin family
member 18A (DKFZP434G2226), mRNA”, gi|21314741|ref|NM_031217.2|[21314741]; 2705:
NM_031219, “Homo sapiens hypothetical protein MGC12904 (MGC12904), mRNA”,
gi|31377665|ref|NM_031219.2|[31377665]; 2706: NM_031231, “Homo sapiens amyloid beta
(A4) precursor protein-binding, family A, member 2”, “binding protein (APBA2BP), transcript
variant 1, mRNA”, gi|38569412|ref|NM_031231.2|[38569412]; 2707: NM_031275, “Homo
sapiens testis expressed sequence 12 (TEX12), mRNA”,
gi|14277686|ref|NM_031275.2|[14277686]; 2708: NM_031280, “Homo sapiens mitochondrial
ribosomal protein S15 (MRPS15), nuclear gene encoding”, “mitochondrial protein, mRNA”,
gi|16554610|ref|NM_031280.2|[16554610]; 2709: NM_031284, “Homo sapiens ATP-dependent
glucokinase (ADP-GK), mRNA”, gi|31542508|ref|NM_031284.3|[31542508]; 2710:
NM_031287, “Homo sapiens splicing factor 3b, subunit 5, 10 kDa (SF3B5), mRNA”,
gi|42740890|ref|NM_031287.2|[42740890]; 2711: NM_031289, “Homo sapiens germ cell
associated 1 (GSG1), mRNA”, gi|13775203|ref|NM_031289.1|[13775203]; 2712: NM_031296,
“Homo sapiens RAB33B, member RAS oncogene family (RAB33B), mRNA”,
gi|13786128|ref|NM_031296.1|[13786128]; 2713: NM_031298, “Homo sapiens hypothetical
protein MGC2963 (MGC2963), mRNA”, gi|13775219|ref|NM_031298.1|[13775219]; 2714:
NM_031299, “Homo sapiens cell division cycle associated 3 (CDCA3), mRNA”,
gi|34147595|ref|NM_031299.3|[34147595]; 2715: NM_031307, “Homo sapiens hypothetical
protein FKSG32 (FKSG32), mRNA”, gi|31542635|ref|NM_031307.2|[31542635]; 2716:
NM_031310 “Homo sapiens plasmalemma vesicle associated protein (PLVAP), mRNA”,
gi|13775237|ref|NM_031310.1|[13775237]; 2717: NM_031450, “Homo sapiens hypothetical
protein p5326 (P5326), mRNA”, gi|31543378|ref|NM_031450.2|[31543378]; 2718: NM_031485,
“Homo sapiens glutamate-rich WD repeat containing 1 (GRWD1), mRNA”,
gi|31542861|ref|NM_031485.2|[31542861]; 2719: NM_031904, “Homo sapiens hypothetical
protein FKSG44 (FKSG44), mRNA”, gi|31982912|ref|NM_031904.2|[31982912]; 2720:
NM_031922 “Homo sapiens RALBP1 associated Eps domain containing 1 (REPS1), mRNA”,
gi|39812393|ref|NM_031922.2|[39812393]; 2721: NM_031966 “Homo sapiens cyclin B1
(CCNB1), mRNA”, gi|34304372|ref|NM_031966.2|[34304372]; 2722: NM_032048, “Homo
sapiens elastin microfibril interfacer 2 (EMILIN2), mRNA”,
gi|14042987|ref|NM_032048.1|[14042987]; 2723: NM_032119, “Homo sapiens monogenic,
audiogenic seizure susceptibility 1 homolog (mouse)”, “(MASS1), mRNA”,
gi|19882212|ref|NM_032119.1|[19882212]; 2724: NM_032144, “Homo sapiens RAB6C,
member RAS oncogene family (RAB6C), mRNA”, gi|14149798|ref|NM_032144.1|[14149798];
2725: NM_032153, “Homo sapiens Zic family member 4 (ZIC4), mRNA”,
gi|22547200|ref|NM_032153.2|[22547200]; 2726: NM_032179, “Homo sapiens hypothetical
protein FLJ20542 (FLJ20542), mRNA”, gi|14149862|ref|NM_032179.1|[14149862]; 2727:
NM_032204, “Homo sapiens ASC-1 complex subunit P100 (ASC1p100), mRNA”,
gi|34147616|ref|NM_032204.3|[34147616]; 2728: NM_032209, “Homo sapiens hypothetical
protein FLJ21777 (FLJ21777), mRNA”, gi|4149905|ref|NM_032209.1|[14149905]; 2729:
NM_032219, “Homo sapiens hypothetical protein FLJ22269 (FLJ22269), mRNA”,
gi|31542730|ref|NM_032219.2|[31542730]; 2730: NM_032233, “Homo sapiens hypothetical
protein FLJ23027 (FLJ23027), transcript variant 1,”, mRNA,
gi|40068480|ref|NM_032233.2|[40068480]; 2731: NM_032338, “Homo sapiens hypothetical
protein MGC14817 (MGC14817), mRNA”, gi|31543151|ref|NM_032338.2|[31543151]; 2732:
NM_032348, “Homo sapiens hypothetical protein MGC3047 (MGC3047), mRNA”,
gi|39725651|ref|NM_032348.2|[39725651]; 2733: NM_032389, “Homo sapiens zinc finger
protein 289, ID1 regulated (ZNF289), mRNA”, gi|31543982|ref|NM_032389.2|[31543982];
2734: NM_032477, “Homo sapiens mitochondrial ribosomal protein L41 (MRPL41), nuclear
gene encoding”, “mitochondrial protein, mRNA”, gi|21265092|ref|NM_032477.1|[21265092];
2735: NM_032509, “Homo sapiens RNA binding protein (LOC84549), mRNA”,
gi|31543090|ref|NM_032509.2|[31543090]; 2736: NM_032569, “Homo sapiens cytokine-like
nuclear factor n-pac (N-PAC), mRNA”, gi|40556375|ref|NM_032569.2|[40556375]; 2737:
NM_032668,, ref|NM_032668.1|[14249231], This record was temporarily removed by RefSeq
staff for additional review.,, 2738: NM_032715,, ref|NM_032715.1|[14249317], This record
was replaced or removed. See revision history for details.,, 2739: NM_032737, “Homo sapiens
lamin B2 (LMNB2), mRNA”, gi|27436950|ref|NM_032737.2|[27436950]; 2740: NM_032765,
“Homo sapiens tripartite motif-containing 52 (TRIMS2), mRNA”,
gi|34147443|ref|NM_032765.2|[34147443]; 2741: NM_032842, “Homo sapiens hypothetical
protein FLJ14803 (FLJ14803), mRNA”, gi|14249557|ref|NM_032842.1|[14249557]; 2742:
NM_032856, “Homo sapiens hypothetical protein FLJ14888 (FLJ14888), mRNA”,
gi|14249585|ref|NM_032856.1|[14249585]; 2743: NM_032865, “Homo sapiens C-terminal
tensin-like (CTEN), mRNA”, gi|23943811|ref|NM_032865.3|[23943811]; 2744: NM_032895,
“Homo sapiens hypothetical protein MGC14376 (MGC14376), mRNA”,
gi|14249657|ref|NM_032895.1|[14249657]; 2745: NM_033211, “Homo sapiens hypothetical
gene supported by AF038182; BC009203 (LOC90355), mRNA”,
gi|34147457|ref|NM_033211.2|[34147457]; 2746: NM_033284, “Homo sapiens transducin
(beta)-like 1Y-linked (TBL1Y), transcript variant 1,”, mRNA,
gi|15150804|ref|NM_033284.1|[15150804]; 2747: NM_033411, “Homo sapiens RWD domain
containing 2 (RWDD2), mRNA”, gi|34222125|ref|NM_033411.2|[34222125]; 2748:
NM_033415, “Homo sapiens hypothetical gene MGC19595 (MGC19595), mRNA”,
gi|16445355|ref|NM_033415.2|[16445355]; 2749: NM_033416, “Homo sapiens U3 snoRNP
protein 4 homolog (IMP4), mRNA”, gi|15529981|ref|NM_033416.1|[15529981]; 2750:
NM_033418, “Homo sapiens hypothetical protein MGC9084 (MGC9084), mRNA”,
gi|15553096|ref|NM_033418.1|[15553096]; 2751: NM_033453, Homo sapiens inosine
triphosphatase (nucleoside triphosphate pyrophosphatase), “(ITPA), transcript variant 1,
mRNA”, gi|31657145|ref|NM_033453.2|[31657145]; 2752: NM_033546, “Homo sapiens
myosin regulatory light chain MRLC2 (MRLC2), mRNA”,
gi|29568092|ref|NM_033546.2|[29568092]; 2753: NM_052940, “Homo sapiens hypothetical
protein MGC8974 (MGC8974), mRNA”, gi|31543202|ref|NM_052940.3|[31543202]; 2754:
NM_079834, “Homo sapiens secretory carrier membrane protein 4 (SCAMP4), mRNA”,
gi|17738286|ref|NM_079834.1|[17738286]; 2755: NM_080839, “Homo sapiens gamma-
glutamyltransferase-like 4 (GGTL4), transcript variant 2,”, mRNA,
gi|40353751|ref|NM_080839.4|[40353751]; 2756: NM_130463, “Homo sapiens ATPase, H+
transporting, lysosomal 13 kDa, V1 subunit G isoform 2”, “(ATP6V1G2), transcript variant 1,
mRNA”, gi|20357536|ref|NM_130463.2|[20357536]; 2757: NM_133455, “Homo sapiens emilin
and multimerin-domain containing protein 1 (EMU1), mRNA”,
gi|19263344|ref|NM_133455.1|[19263344]; 2758: NM_138288, “Homo sapiens chromosome 14
open reading frame 147 (C14orf147), mRNA”, gi|19923718|ref|NM_138288.1|[19923718];
2759: NM_138402, “Homo sapiens hypothetical protein BC004921 (LOC93349), mRNA”,
gi|20149710|ref|NM_138402.2|[20149710]; 2760: NM_138570, “Homo sapiens hypothetical
protein MGC15523 (MGC15523), mRNA”, gi|20070375|ref|NM_138570.1|[20070375]; 2761:
NM_139136, “Homo sapiens potassium voltage-gated channel, Shaw-related subfamily,
member 2”, “(KCNC2), transcript variant 1, mRNA”,
gi|24497456|ref|NM_139136.2|[24497456]; 2762: NM_139170, “Homo sapiens hypothetical
protein AF447587 (LOC146562), mRNA”, gi|21040258|ref|NM_139170.1|[21040258]; 2763:
NM_139246, “Homo sapiens PP4189 (LOC158427), mRNA”,
gi|31377600|ref|NM_139246.3|[31377600]; 2764: NM_139265, “Home sapiens EH-domain
containing 4 (EHD4), mRNA”, gi|34147619|ref|NM_139265.2|[34147619]; 2765: NM_144617,
“Homo sapiens hypothetical protein FLJ32389 (FLJ32389), mRNA”,
gi|21389432|ref|NM_144617.1|[21389432]; 2766: NM_144635, “Homo sapiens hypothetical
protein MGC21688 (MGC21688), mRNA”, gi|40255250|ref|NM_144635.3|[40255250]; 2767:
NM_144718, “Homo sapiens hypothetical protein AY099107 (LOC152185), mRNA”,
gi|40255074|ref|NM_144718.2|[40255074]; 2768: NM_145060, “Homo sapiens hypothetical
protein MGC: 10200 (MGC10200), mRNA”, gi|21450831|ref|NM_145060.1|[21450831]; 2769:
NM_145063, “Homo sapiens chromosome 6 open reading frame 130 (C6orf130), mRNA”,
gi|34147711|ref|NM_145063.2|[34147711]; 2770: NM_145804, “Home sapiens ankyrin repeat
and BTB (POZ) domain containing 2 (ABTB2), mRNA”,
gi|21956638|ref|NM_145804.1|[21956638]; 2771: NM_147129, “Homo sapiens hypothetical
protein LOC259173 (FLJ36525), transcript variant 1,”, mRNA,
gi|33359214|ref|NM_147129.2|[33359214]; 2772: NM_152272, “Home sapiens hypothetical
protein MGC29816 (MGC29816), mRNA”, gi|22748640|ref|NM_152272.1|[22748640]; 2773:
NM_152275, “Homo sapiens hypothetical protein FLJ13946 (FLJ13946), mRNA”,
gi|38683852|ref|NM_152275.2|[38683852]; 2774: NM_152288, “Home sapiens hypothetical
protein MGC13024 (MGC13024), mRNA”, gi|22748650|ref|NM_152288.1|[22748650]; 2775:
NM_152339, “Homo sapiens hypothetical protein MGC26885 (MGC26885), mRNA”,
gi|31377584|ref|NM_152339.2|[31377584]; 2776: NM_152341, “Homo sapiens hypothetical
protein FLJ30002 (FLJ3 0002), mRNA”, gi|31542755|ref|NM_52341.2|[31542755]; 2777:
NM_152519, “Homo sapiens hypothetical protein FLJ23861 (FLJ23861), mRNA”,
gi|40217793|ref|NM_152519.2|[40217793]; 2778: NM_152647, “Home sapiens hypothetical
protein FLJ32800 (FLJ32800), mRNA”, gi|22749318|ref|NM_152647.1|[22749318]; 2779:
NM_152660, “Homo sapiens hypothetical protein MGC34648 (MGC34648), mRNA”,
gi|22749340|ref|NM_152660.1|[22749340]; 2780: NM_152688, “Homo sapiens KH domain
containing, RNA binding, signal transduction associated 2”, “(KHDRBS2), mRNA”,
gi|22749380|ref|NM_152688.1|[22749380]; 2781: NM_152703,, ref|NM_152703.1|[22749402],
This record was temporarily removed by RefSeq staff for additional review.,, 2782:
NM_152726, “Homo sapiens Smhs2 homolog (rat) (FLJ34588), mRNA”,
gi|22749442|ref|NM_152726.1|[22749442]; 2783: NM_152753, “Homo sapiens CUB domain
and EGF-like repeat containing 3 (CEGF3), mRNA”,
gi|31377567|ref|NM_152753.2|[31377567]; 2784: NM_152754, “Homo sapiens sema domain,
immunoglobulin domain (Ig), short basic domain,”, “secreted, (semaphorin) 3D (SEMA3D),
mRNA”, gi|41406085|ref|NM_152754.2|[41406085]; 2785: NM_152758, “Homo sapiens
hypothetical protein FLJ31657 (FLJ31657), mRNA”,
gi|40255334|ref|NM_152758.2|[40255134]; 2786: NM_152769, “Homo sapiens hypothetical
protein MGC40084 (MGC40084), mRNA”, gi|22749502|ref|NM_152769.1|[22749502]; 2787:
NM_152902, “Homo sapiens putative MAPK activating protein (MGC3794), mRNA”,
gi|33239373|ref|NM_152902.2|[33239373]; 2788: NM_153045, “Homo sapiens
DKFZp547P234 protein (DKFZp547P234), mRNA”,
gi|33356141|ref|NM_153045.2|[33356141]; 2789: NM_153354, “Homo sapiens hypothetical
protein MGC33214 (MGC33214), mRNA”, gi|34222213|ref|NM_153354.2|[34222213]; 2790:
NM_153603, “Homo sapiens component of oligomeric golgi complex 7 (COG7), mRNA”,
gi|23957689|ref|NM_153603.1|[23957689]; 2791: NM_153811, “Homo sapiens solute carrier
family 38, member 6 (SLC38A6), mRNA”, gi|24429573|ref|NM_153811.1|[24429573]; 2792:
NM_172341, “Homo sapiens presenilin enhancer 2 (PEN2), mRNA”,
gi|28144919|ref|NM_172341.1|[28144919]; 2793: NM_173481, “Homo sapiens hypothetical
protein LOC126353 (LOC126353), mRNA”, gi|34222226|ref|NM_173481.2|[34222226]; 2794:
NM_173500, “Homo sapiens tau tubulin kinase 2 (TTBK2), mRNA”,
gi|28466990|ref|NM_173500.2|[28466990]; 2795: NM_173509, “Homo sapiens hypothetical
protein MGC16664 (MGC16664), mRNA”, gi|34222229|ref|NM_173509.2|[34222229]; 2796:
NM_173562, “Homo sapiens chromosome 6 open reading frame 69 (C6orf69), mRNA”,
gi|40255181|ref|NM_173562.3|[40255181]; 2797: NM_175066, “Homo sapiens DEAD (Asp-
Glu-Ala-Asp) box polypeptide 51 (DDX51), mRNA”,
gi|37059776|ref|NM_175066.2|[37059776]; 2798: NM_175886, “Homo sapiens phosphoribosyl
pyrophosphate synthetase 1-like 1 (PRPS1L1), mRNA”,
gi|31343499|ref|NM_175886.2|[31343499]; 2799: NM_177966, “Homo sapiens hypothetical
protein DKFZp667B1218 (DKFZp667B1218), mRNA”,
gi|34222255|ref|NM_177966.3|[34222255],,

TABLE 13
Genes having both an Errα binding motif and a Gabpa binding motif

1: NM_000164, “Homo sapiens gastric inhibitory polypeptide receptor (GIPR), mRNA”,
gi|4503998|ref|NM_000164.1|[4503998]; 2: NM_000183, Homo sapiens hydroxyacyl-
Coenzyme A dehydrogenase/3-ketoacyl-Coenzyme A, “thiolase/enoyl-Coenzyme A hydratase
(trifunctional protein), beta subunit”, “(HADHB), mRNA”,
gi|4504326|ref|NM_000183.1|[4504326]; 3: NM_000249, “Homo sapiens mutL homolog 1,
colon cancer, nonpolyposis type 2 (E. coli) (MLH1),”, mRNA,
gi|28559089|ref|NM_000249.2|[28559089]; 4: NM_000274, “Homo sapiens omithine
aminotransferase (gyrate atrophy) (OAT), nuclear gene”, “encoding mitoohondrial protein,
mRNA”, gi|4557808|ref|NM_000274.1|[4557808]; 5: NM_000297, “Homo sapiens polycystic
kidney disease 2 (autosomal dominant) (PKD2), mRNA”,
gi|33286447|ref|NM_000297.2|[33286447]; 6: NM_000347, “Homo sapiens spectrin, beta,
erythrocytic (includes spherocytosis, clinical type”, “I) (SPTB), mRNA”,
gi|22507315|ref|NM_000347.3|[22507315]; 7: NM_000364, “Homo sapiens troponin T2,
cardiac (TNNT2), mRNA”, gi|4507626|ref|NM_000364.1|[4507626]; 8: NM_000403, “Homo
sapiens galactose-4-epimerase, UDP (GALE), mRNA”,
gi|9945333|ref|NM_000403.2|[9945333]; 9: NM_000474, Homo sapiens twist homolog 1
(acrocephalosyndactyly 3; Saethre-Chotzen syndrome), “(Drosophila) (TWIST1), mRNA”,
gi|17978464|ref|NM_000474.2|[17978464]; 10: NM_000483, “Homo sapiens apolipoprotein C-
II (APOC2), mRNA”, gi|32130517|ref|NM_000483.3|[32130517]; 11: NM_000499, “Homo
sapiens cytochrome P450, family 1, subfamily A, polypeptide 1 (CYP1A1),”, mRNA,
gi|13325053|ref|NM_000499.2|[13325053]; 12: NM_000526, “Homo sapiens keratin 14
(epidermolysis bullosa simplex, Dowling-Meara, Koebner)”, “(KIRT14), mRNA”,
gi|15431309|ref|NM_000526.3|[15431309]; 13: NM_000593, “Homo sapiens transporter 1,
ATP-binding cassette, sub-family B (MDR/TAP) (TAP1),”, mRNA,
gi|24797159|ref|NM_000593.4|[24797159]; 14: NM_000603, “Homo sapiens nitric oxide
synthase 3 (endothelial cell) (NOS3), mRNA”, gi|40254421|ref|NM_000603.2|[40254421]; 15:
NM_000628, “Homo sapiens interleukin 10 receptor, beta (IL10RB), mRNA”,
gi|24430214|ref|NM_000628.3|[24430214]; 16: NM_000688, “Homo sapiens aminolevulinate,
delta-, synthase 1 (ALAS1), transcript variant 1,”, mRNA,
gi|40316942|ref|NM_000688.4|[40316942]; 17: NM_000747, “Homo sapiens cholinergic
receptor, nicotinic, beta polypeptide 1 (muscle)”, “(CHRNB1), mRNA”,
gi|41327725|ref|NM_000747.2|[41327725]; 18: NM_000781, “Homo sapiens cytoehrome P450,
family 11, subfamily A, polypeptide 1 (CYP11A1),”, “nuclear gene encoding mitochondrial
protein, mRNA”, gi|4503188|ref|NM_000781.1|[4503188]; 19: NM_000806, “Homo sapiens
gamma-aminobutyric acid (GABA) A receptor, alpha 1 (GABRA1), mRNA”,
gi|38327553|ref|NM_000806.3|[38327553]; 20: NM_000813, “Homo sapiens gamma-
aminobutyric acid (GABA) A receptor, beta 2 (GABRB2),”, “transcript variant 2, mRNA”,
gi|4503864|ref|NM_000813.1|[4503864]; 21: NM_000835, “Homo sapiens glutamate receptor,
ionotropic, N-methyl D-aspartate 2C (GRIN2C),”, mRNA,
gi|6006004|ref|NM_000835.2|[6006004]; 22: NM_000915, “Homo sapiens oxytocin, prepro-
(neurophysin I) (OXT), mRNA”, gi|12707574|ref|NM_000915.2|[12707574]; 23: NM_000932,
“Homo sapiens phospholipase C, beta 3 (phosphatidylinositol-specific) (PLCB3),”, mRNA,
gi|11386138|ref|NM_000932.1|[11386138]; 24: NM_001040, “Homo sapiens sex hormone-
binding globulin (SHBG), mRNA”, gi|7382459|ref|NM_001040.2|[7382459]; 25: NM_001087,
“Homo sapiens angio-associated, migratory cell protein (AAMP), mRNA”,
gi|4557228|ref|NM_001087.1|[4557228]; 26: NM_001094, “Homo sapiens amiloride-sensitive
cation channel 1, neuronal (degenerin) (ACCN1),”, “transcript variant 2, mRNA”,
gi|34452696|ref|NM_001094.4|[34452696]; 27: NM_001099, “Homo sapiens acid phosphatase,
prostate (ACPP), mRNA”, gi|6382063|ref|NM_001099.2|[6382063]; 28: NM_001104, “Homo
sapiens actinin, alpha 3 (ACTN3), mRNA”, gi|4557240|ref|NM_001104.1|[4557240]; 29:
NM_001158, “Homo sapiens amine oxidase, copper containing 2 (retina-specific) (AOC2),”,
“transcript variant 1, mRNA”, gi|6806880|ref|NM_001158.2|[6806880]; 30: NM_001164,
“Homo sapiens amyloid beta (A4) precursor protein-binding, family B, member 1”, “(Fe65)
(APBB1), transcript variant 1, mRNA”, gi|22035552|ref|NM_001164.2|[22035552]; 31:
NM_001188, “Homo sapiens BCL2-antagonist/killer 1 (BAK1), mRNA”,
gi|33457353|ref|NM_001188.2|[33457353]; 32: NM_001257, “Homo sapiens cadherin 13, H-
cadherin (heart) (CDH13), mRNA”, gi|16507956|ref|NM_001257.2|[16507956]; 33:
NM_001261, “Homo sapiens cyclin-dependent kinase 9 (CDC2-related kinase) (CDK9),
mRNA”, gi|17017983|ref|NM_001261.2|[17017983]; 34: NM_001425, “Homo sapiens
epithelial membrane protein 3 (EMP3), mRNA”, gi|4503562|ref|NM_001425.1|[4503562]; 35:
NM_001501, “Homo sapiens gonadotropin-releasing hormone 2 (GNRH2), transcript variant
1,”, mRNA, gi|4504056|ref|NM_001501.1|[4504056]; 36: NM_001542, “Homo sapiens
immunoglobulin superfamily, member 3 (IGSF3), mRNA”,
gi|4504626|ref|NM_001542.1|[4504626]; 37: NM_001662, “Homo sapiens ADP-ribosylation
factor 5 (ARF5), mRNA”, gi|6995999|ref|NM_001662.2|[6995999]; 38: NM_001666, “Homo
sapiens Rho GTPase activating protein 4 (ARHGAP4), mRNA”,
gi|41327157|ref|NM_001666.2|[41327157]; 39: NM_001702, “Homo sapiens brain-specific
angiogenesis inhibitor 1 (BAI1), mRNA”, gi|4502354|ref|NM_001702.1|[4502354]; 40:
NM_001722, “Homo sapiens polymerase (RNA) III (DNA directed) polypeptide D, 44 kDa
(POLR3D),”, mRNA, gi|4502436|ref|NM_001722.1|[4502436]; 41: NM_001823, “Homo
sapiens creatine kinase, brain (CKB), mRNA”, gi|34335231|ref|NM_001823.3|[34335231]; 42:
NM_001859, “Homo sapiens solute carrier family 31 (copper transporters), member 1
(SLC31A1),”, mRNA, gi|40254457|ref|NM_001859.2|[40254457]; 43: NM_001864, “Homo
sapiens cytochrome c oxidase subunit VIIa polypeptide 1 (muscle) (COX7A1),”, mRNA,
gi|18105034|ref|NM_001864.2|[18105034]; 44: NM_001887, “Homo sapiens crystallin, beta B1
(CRYBB1), mRNA”, gi|21536279|ref|NM_001887.3|[21536279]; 45: NM_001893, “Homo
sapiens casein kinase 1, delta (CSNK1D), transcript variant 1, mRNA”,
gi|20544143|ref|NM_001893.3|[20544143]; 46: NM_001895, “Homo sapiens casein kinase 2,
alpha 1 polypeptide (CSNK2A1), transcript variant”, “2, mRNA”,
gi|29570794|ref|NM_001895.2|[29570794]; 47: NM_001923, “Homo sapiens damage-specific
DNA binding protein 1, 127 kDa (DDB1), mRNA”, gi|13435358|ref|NM_001923.2|[13435358];
48: NM_001958, “Homo sapiens eukaryotic translation elongation factor 1 alpha 2 (EEF1A2),
mRNA”, gi|25453470|ref|NM_001958.2|[25453470]; 49: NM_002010, “Homo sapiens
fibroblast growth factor 9 (glia-activating factor) (FGF9), mRNA”,
gi|4503706|ref|NM_002010.1|[4503706]; 50: NM_002012, “Homo sapiens fragile histidine triad
gene (FHIT), mRNA”, gi|4503718|ref|NM_002012.1|[4503718]; 51: NM_002083, “Homo
sapiens glutathione peroxidase 2 (gastrointestinal) (GPX2), mRNA”,
gi|32967606|ref|NM_002083.2|[32967606]; 52: NM_002151, “Homo sapiens hepsin
(transmembrane protease, serine 1) (HPN), transcript variant”, “2, mRNA”,
gi|4504480|ref|NM_002151.1|[4504480]; 53: NM_002157, “Homo sapiens heat shock 10 kDa
protein 1 (chaperonin 10) (HSPE1), mRNA”, gi|4504522|ref|NM_002157.1|[4504522]; 54:
NM_002193, “Homo sapiens inhibin, beta B (activin AB beta polypeptide) (INHBB), mRNA”,
gi|9257224|ref|NM_002193.1|[9257224]; 55: NM_002217, “Homo sapiens pre-alpha (globulin)
inhibitor, H3 polypeptide (ITIH3), mRNA”, gi|10092578|ref|NM_002217.1|[10092578]; 56:
NM_002238, “Homo sapiens potassium voltage-gated channel, subfamily H (eag-related),
member”, “1 (KCNH1), transcript variant 2, mRNA”,
gi|27436999|ref|NM_002238.2|[27436999]; 57: NM_002257, “Homo sapiens kallikrein 1,
renal/pancreas/salivary (KLK1), mRNA”, gi|22027643|ref|NM_002257.2|[22027643]; 58:
NM_002280, “Homo sapiens keratin, hair, acidic, 5 (KRTHA5), mRNA”,
gi|15431313|ref|NM_002280.3|[15431313]; 59: NM_002378, “Homo sapiens megakaryocyte-
associated tyrosine kinase (MATK), transcript variant”, “2, mRNA”,
gi|21450841|ref|NM_002378.2|[21450841]; 60: NM_002419, “Homo sapiens mitogen-activated
protein kinase kinase kinase 11 (MAP3K11), mRNA”,
gi|21735553|ref|NM_002419.2|[21735553]; 61: NM_002437, “Homo sapiens MpV17 transgene,
murine homolog, glomerulosclerosis (MPV17), mRNA”,
gi|37059781|ref|NM_002437.3|[37059781]; 62: NM_002492, “Homo sapiens NADH
dehydrogenase (ubiquinone) 1 beta subcomplex, 5, 16 kDa”, “(NDUFB5), nuclear gene encoding
mitochondrial protein, mRNA”, gi|33519467|ref|NM_002492.2|[33519467]; 63: NM_002506,
“Homo sapiens nerve growth factor, beta polypeptide (NGFB), mRNA”,
gi|4505390|ref|NM_002506.1|[4505390]; 64: NM_002590, “Homo sapiens protocadherin 8
(PCDH8), transcript variant 1, mRNA”, gi|6631101|ref|NM_002590.2|[6631101]; 65:
NM_002599, “Homo sapiens phosphodiesterase 2A, cGMP-stimulated (PDE2A), mRNA”,
gi|4505656|ref|NM_002599.1|[4505656]; 66: NM_002630, “Homo sapiens progastricsin
(pepsinogen C) (PGC), mRNA”, gi|4505756|ref|NM_002630.1|[4505756]; 67: NM_002831,
“Homo sapiens protein tyrosine phosphatase, non-receptor type 6 (PTPN6),”, “transcript variant
1, mRNA”, gi|34328900|ref|NM_002831.3|[34328900]; 68: NM_002832, “Homo sapiens
protein tyrosine phosphatase, non-receptor type 7 (PTPN7),”, “transcript variant 1, mRNA”,
gi|18375657|ref|NM_002832.2|[18375657]; 69: NM_002904, “Homo sapiens RD RNA binding
protein (RDBP), mRNA”, gi|20631983|ref|NM_002904.4|[20631983]; 70: NM_002912, “Homo
sapiens REV3-like, catalytic subunit of DNA polymerase zeta (yeast)”, “(REV3L), mRNA”,
gi|4506482|ref|NM_002912.1|[4506482]; 71: NM_002930, “Homo sapiens Ras-like without
CAAX 2 (RIT2), mRNA”, gi|4506532|ref|NM_002930.1|[4506532]; 72: NM_002938, “Homo
sapiens ring finger protein 4 (RNF4), mRNA”, gi|34305289|ref|NM_002938.2|[34305289]; 73:
NM_002965, “Homo sapiens S100 calcium binding protein A9 (calgranulin B) (S100A9),
mRNA”, gi|9845520|ref|NM_002965.2|[9845520]; 74: NM_003002, “Homo sapiens succinate
dehydrogenase complex, subunit D, integral membrane”, “protein (SDHD), nuclear gene
encoding mitochondrial protein, mRNA”, gi|4506864|ref|NM_003002.1|[4506864]; 75:
NM_003042, “Homo sapiens solute carrier family 6 (neurotransmitter transporter, GABA),”,
“member 1 (SLC6A1), mRNA”, gi|40254466|ref|NM_003042.2|[40254466]; 76: NM_003055,
“Homo sapiens solute carrier family 18 (vesicular acetylcholine), member 3”, “(SLC18A3),
mRNA”, gi|4506990|ref|NM_003055.1|[4506990]; 77: NM_003115, “Homo sapiens UDP-N-
acteylglucosamine pyrophosphorylase 1 (UAP1), mRNA”,
gi|34147515|ref|NM_003115.3|[34147515]; 78: NM_003159, “Homo sapiens cyclin-dependent
kinase-like 5 (CDKL5), mRNA”, gi|4507280|ref|NM_003159.1|[4507280]; 79: NM_003216,
“Homo sapiens thyrotrophic embryonic factor (TEF), mRNA”,
gi|34486096|ref|NM_003216.2|[34486096]; 80: NM_003239, “Homo sapiens transforming
growth factor, beta 3 (TGFB3), mRNA”, gi|4507464|ref|NM_003239.1|[4507464]; 81:
NM_003259, “Homo sapiens intercellular adhesion molecule 5, telencephalin (ICAM5),
mRNA”, gi|12545403|ref|NM_003259.2|[12545403]; 82: NM_003325, Homo sapiens HIR
histone cell cycle regulation defective homolog A (S., “cerevisiae) (HIRA), mRNA”,
gi|21536484|ref|NM_003325.3|[21536484]; 83: NM_003334, Homo sapiens ubiquitin-activating
enzyme E1 (A1S9T and BN75 temperature, “sensitivity complementing) (UBE1), transcript
variant 1, mRNA”, gi|23510337|ref|NM_003334.2|[23510337]; 84: NM_003341, “Homo
sapiens ubiquitin-conjugating enzyme E2E 1 (UBC4/5 homolog, yeast)”, “(UBE2E1), transcript
variant 1, mRNA”, gi|33359692|ref|NM_003341.3|[33359692]; 85: NM_003374, “Homo
sapiens voltage-dependent anion channel 1 (VDAC1), mRNA”,
gi|4507878|ref|NM_003374.1|[4507878]; 86: NM_003418, Homo sapiens zinc finger protein 9
(a cellular retroviral nucleic acid binding, “protein) (ZNF9), mRNA”,
gi|4827070|ref|NM_003418.1|[4827070]; 87: NM_003492, “Homo sapiens chromosome X open
reading frame 12 (CXorf12), mRNA”, gi|4504738|ref|NM_003492.1|[4504738]; 88: NM_003524,
“Homo sapiens histone 1, H2bh (HIST1H2BH), mRNA”,
gi|21166386|ref|NM_003524.2|[21166386]; 89: NM_003549, “Homo sapiens
hyaluronoglucosaminidase 3 (HYAL3), mRNA”, gi|15208650|ref|NM_003549.2|[15208650]; 90:
NM_003554, “Homo sapiens olfactory receptor, family 1, subfamily E, member 2 (OR1E2),
mRNA”, gi|11386152|ref|NM_003554.1|[11386152]; 91: NM_003594, “Homo sapiens
transcription termination factor, RNA polymerase II (TTF2), mRNA”,
gi|40807470|ref|NM_003594.3|[40807470]; 92: NM_003627, “Homo sapiens solute carrier
family 43, member 1 (SLC43A1), mRNA”, gi|42476323|ref|NM_003627.4|[42476323]; 93:
NM_003632, “Homo sapiens contactin associated protein 1 (CNTNAP1), mRNA”,
gi|4505462|ref|NM_003632.1|[4505462]; 94: NM_003691, “Homo sapiens serine/threonine
kinase 16 (STK16), mRNA”, gi|4505836|ref|NM_003691.1|[4505836]; 95: NM_003860, “Homo
sapiens barrier to autointegration factor 1 (BANF1), mRNA”,
gi|11038645|ref|NM_003860.2|[11038645]; 96: NM_003957, “Homo sapiens serine/threonine
kinase 29 (STK29), mRNA”, gi|27501463|ref|NM_003957.1|[27501463]; 97: NM_004074,
“Homo sapiens cytochrome c oxidase subunit VIII (COX8), mRNA”,
gi|4758043|ref|NM_004074.1|[4758043]; 98: NM_004078, “Homo sapiens cysteine and glycine-
rich protein 1 (CSRP1), mRNA”, gi|4758085|ref|NM_004078.1|[4758085]; 99: NM_004100,
“Homo sapiens eyes absent homolog 4 (Drosophila) (EYA4), transcript variant 1, ”, mRNA,
gi|26667248|ref|NM_004100.2|[26667248]; 100: NM_004106, “Homo sapiens Fc fragment of
IgE, high affinity I, receptor for; gamma”, “polypeptide (FCER1G), mRNA”,
gi|4758343|ref|NM_004106.1|[4758343]; 101: NM_004178, “Homo sapiens TAR (HIV) RNA
binding protein 2 (TARBP2), transcript variant 3,”, mRNA,
gi|19743837|ref|NM_004178.3|[19743837]; 102: NM_004260, “Homo apiens RecQ protein-
like 4 (RECQL4), mRNA”, gi|4759029|ref|NM_004260.1|[4759029]; 103: NM_004267, “Homo
sapiens carbohydrate (N-acetylglucosamine-6-O) sulfotransferase 2 (CHST2), ”, mRNA,
gi|27369496|ref|NM_004267.2|[27369496]; 104: NM_004294, “Homo sapiens mitochondrial
translational release factor 1 (MTRF1), nuclear gene”, “encoding mitochondrial protein, mRNA”,
gi|34577119|ref|NM_004294.2|[34577119]; 105: NM_004344, “Homo sapiens centrin, EF-hand
protein, 2 (CETN2), mRNA”, gi|4757901|ref|NM_004344.1|[4757901]; 106: NM_004358,
“Homo sapiens cell division cycle 25B (CDC25B), transcript variant 1, mRNA”,
gi|11641416|ref|NM_004358.2|[11641416]; 107: NM_004374, “Homo sapiens cytochrome c
oxidase subunit VIc (COX6C), mRNA”, gi|17999531|ref|NM_004374.2|[17999531]; 108:
NM_004427, “Homo sapiens polyhomeotic-like 2 (Drosophila) (PHC2), transcript variant 2,
mRNA”, gi|37595529|ref|NM_004427.2|[37595529]; 109: NM_004470, “Homo sapiens FK506
binding protein 2, 13 kDa (FKBP2), transcript variant 1, mRNA”,
gi|17149841|ref|NM_004470.2|[17149841]; 110: NM_004528, “Homo sapiens microsomal
glutathione S-transferase 3 (MGST3), mRNA”, gi|22035640|ref|NM_004528.2|[22035640]; 111:
NM_004550, “Homo sapiens NADH dehydrogenase (ubiquinone) Fe—S protein 2, 49 kDa”,
“(NADH-coenzyme Q reductase) (NDUFS2), mRNA”,
gi|34147556|ref|NM_004550.3|[34147556]; 112: NM_004604, “Homo sapiens syntaxin 4A
(placental) (STX4A), mRNA”, gi|34147603|ref|NM_004604.3|[34147603]; 113: NM_004656,
Homo sapiens BRCA1 associated protein-1 (ubiquitin carboxy-terminal hydrolase), “(BAP1),
mRNA”, gi|19718752|ref|NM_004656.2|[19718752]; 114: NM_004672, “Homo sapiens
mitogen-activated protein kinase kinase kinase 6 (MAP3K6),”, “transcript variant 1, mRNA”,
gi|24497521|ref|NM_004672.2|[24497521]; 115: NM_004704, “Homo sapiens RNA, U3 small
nucleolar interacting protein 2 (RNU3IP2), mRNA”, gi|31543556|ref|NM_004704.2|[31543556];
116: NM_004870, “Homo sapiens mannose-P-dolichol utilization defect 1 (MPDU1), mRNA”,
gi|4759109|ref|NM_004870.1|[4759109]; 117: NM_004913, “Homo sapiens chromosome 16
open reading frame 7 (C16orf7), mRNA”, gi|4757805|ref|NM_004913.1|[4757805]; 118:
NM_004927, “Homo sapiens mitochondrial ribosomal protein L49 (MRPL49), nuclear gene
encoding”, “mitochondrial protein, mRNA”, gi|27436906|ref|NM_004927.2|[27436906]; 119:
NM_004941, “Homo sapiens DEAH (Asp-Glu-Ala-His) box polypeptide 8 (DHX8), mRNA”,
gi|4826689|ref|NM_004941.1|[4826689]; 120: NM_004959, “Homo sapiens nuclear receptor
subfamily 5, group A, member 1 (NR5A1), mRNA”, gi|24432033|ref|NM_004959.3|[24432033];
121: NM_004987, “Homo sapiens LIM and senescent cell antigen-like domains 1 (LIMS1),
mRNA”, gi|13518025|ref|NM_004987.2|[13518025]; 122: NM_004994, “Homo sapiens matrix
metalloproteinase 9 (gelatinase B, 92 kDa gelatinase, 92 kDa”, “type IV collagenase) (MMP9),
mRNA”, gi|4826835|ref|NM_004994.1|[4826835]; 123: NM_005006, “Homo sapiens NADH
dehydrogenase (ubiquinone) Fe—S protein 1, 75 kDa”, “(NADH-coenzyme Q reductase)
(NDUFS1), nuclear gene encoding mitochondrial”, “protein, mRNA”,
gi|33519474|ref|NM_005006.5|[33519474]; 124: NM_005023, “Homo sapiens protein
geranylgeranyltransferase type I, beta subunit (PGGT1B),”, mRNA,
gi|27597101|ref|NM_005023.2|[27597101]; 125: NM_005027, “Homo sapiens
phosphoinositide-3-kinase, regulatory subunit, polypeptide 2 (p85”, “beta) (PIK3R2), mRNA”,
gi|4826907|ref|NM_005027.1|[4826907]; 126: NM_005124, “Homo sapiens nucleoporin
153 kDa (NUP153), mRNA”, gi|24430145|ref|NM_005124.2|[24430145]; 127: NM_005125,
“Homo sapiens copper chaperone for superoxide dismutase (CCS), mRNA”,
gi|4826664|ref|NM_005125.1|[4826664]; 128: NM_005154, “Homo sapiens ubiquitin specific
protease 8 (USP8), mRNA”, gi|41281375|ref|NM_005154.2|[41281375]; 129: NM_005161,
“Homo sapiens angiotensin II receptor-like 1 (AGTRL1), mRNA”,
gi|34577064|ref|NM_005161.2|[34577064]; 130: NM_005182, “Homo sapiens carbonic
anhydrase VII (CA7), mRNA”, gi|4885100|ref|NM_005182.1|[4885100]; 131: NM_005186,
“Homo sapiens calpain 1, (mu/I) large subunit (CAPN1), mRNA”,
gi|12408655|ref|NM_005186.2|[12408655]; 132: NM_005223, “Homo sapiens
deoxyribonuclease I (DNASE1), mRNA”, gi|21361253|ref|NM_005223.2|[21361253]; 133:
NM_005260, “Homo sapiens growth differentiation factor 9 (GDF9), mRNA”,
gi|6715598|ref|NM_005260.2|[6715598]; 134: NM_005286, “Homo sapiens G protein-coupled
receptor 8 (GPR8), mRNA”, gi|30581163|ref|NM_005286.2|[30581163]; 135: NM_005288,
“Homo sapiens G protein-coupled receptor 12 (GPR12), mRNA”,
gi|4885294|ref|NM_005288.1|[4885294]; 136: NM_005302, Homo sapiens G protein-coupled
receptor 37 (endothelin receptor type B-like), “(GPR37), mRNA”,
gi|31377788|ref|NM_005302.2|[31377788]; 137: NM_005306, “Homo sapiens G protein-
coupled receptor 43 (GPR43), mRNA”, gi|4885332|ref|NM_005306.1|[4885332]; 138:
NM_005341, “Homo sapiens GLI-Kruppel family member HKR3 (HKR3), mRNA”,
gi|4885418|ref|NM_005341.1|[4885418]; 139: NM_005393, “Homo sapiens plexin B3
(PLXNB3), mRNA”, gi|10864080|ref|NM_005393.1|[10864080]; 140: NM_005398, “Homo
sapiens protein phosphatase 1, regulatory (inhibitor) subunit 3C (PPP1R3C),”, mRNA,
gi|42476161|ref|NM_005398.3|[42476161]; 141: NM_005418, “Homo sapiens suppression of
tumorigenicity 5 (ST5), transcript variant 1, mRNA”,
gi|121264611|ref|NM_005418.2|[21264611]; 142: NM_005453, “Homo sapiens zinc finger
protein 297 (ZNF297), mRNA”, gi|20070223|ref|NM_005453.3|[20070223]; 143: NM_005475,
“Homo sapiens lymphocyte adaptor protein (LNK), mRNA”,
gi|4885454|ref|NM_005475.1|[4885454]; 144: NM_005485, Homo sapiens ADP-
ribosyltransferase (NAD+; poly (ADP-ribose) polymerase)-like 3, “(ADPRTL3), mRNA”,
gi|11496992|ref|NM_005485.2|[11496992]; 145: NM_005550, “Homo sapiens kinesin family
member C3 (KIFC3), mRNA”, gi|19923320|ref|NM_005550.2|[19923320]; 146: NM_005560,
“Homo sapiens laminin, alpha 5 (LAMA5), mRNA”, gi|21264601|ref|NM_005560.3|[21264601];
147: NM_005563, “Homo sapiens stathmin 1/oncoprotein 18 (STMN1), mRNA”,
gi|13518023|ref|NM_005563.2|[13518023]; 148: NM_005626, “Homo sapiens splicing factor,
arginine/serine-rich 4 (SFRS4), mRNA”, gi|34147660|ref|NM_005626.3|[34147660]; 149:
NM_005634, “Homo sapiens SRY (sex determining region Y)-box 3 (SOX3), mRNA”,
gi|30061555|ref|NM_005634.2|[30061555]; 150: NM_005698, “Homo sapiens secretory carrier
membrane protein 3 (SCAMP3), transcript variant”, “1, mRNA”,
gi|16445418|ref|NM_005698.2|[16445418]; 151: NM_005716, Homo sapiens regulator of G-
protein signalling 19 interacting protein 1, “(RGS19IP1), transcript variant 1, mRNA”,
gi|42544147|ref|NM_005716.2|[42544147]; 152: NM_005726, “Homo sapiens Ts translation
elongation factor, mitochondrial (TSFM), mRNA”, gi|21361279|ref|NM_005726.2|[21361279];
153: NM_005727, “Homo sapiens tetraspan 1 (TSPAN-1), mRNA”,
gi|21264577|ref|NM_005727.2|[21264577]; 154: NM_005845, “Homo sapiens ATP-binding
cassette, sub-family C (CFTR/MRP), member 4 (ABCC4),”, mRNA,
gi|34452699|ref|NM_005845.2|[34452699]; 155: NM_005860, “Homo sapiens follistatin-like 3
(secreted glycoprotein) (FSTL3), mRNA”, gi|5031700|ref|NM_005860.1|[5031700]; 156:
NM_005909, “Homo sapiens microtubule-associated protein 1B (MAP1B), transcript variant
1,”, mRNA, gi|14165457|ref|NM_005909.2|[14165457]; 157: NM_005965, “Homo sapiens
myosin, light polypeptide kinase (MYLK), transcript variant 6, mRNA”,
gi|16950600|ref|NM_005965.2|[16950600]; 158: NM_005984, Homo sapiens solute carrier
family 25 (mitochondrial carrier; citrate, “transporter), member 1 (SLC25A1), mRNA”,
gi|21389314|ref|NM_005984.1|[21389314]; 159: NM_006017, “Homo sapiens prominin 1
(PROM1), mRNA”, gi|5174386|ref|NM_006017.1|[5174386]; 160: NM_006067, “Homo
sapiens neighbor of COX4 (NOC4), mRNA”, gi|34147520|ref|NM_006067.3|[34147520]; 161:
NM_006090, “Homo sapiens choline/ethanolaminephosphotransferase (CEPT1), mRNA”,
gi|21735567|ref|NM_006090.2|[21735567]; 162: NM_006091, “Homo sapiens coronin, actin
binding protein, 2B (CORO2B), mRNA”, gi|24307902|ref|NM_006091.1|[24307902]; 163:
NM_006114, Homo sapiens translocase of outer mitochondrial membrane 40 homolog (yeast),
“(TOMM40), mRNA”, gi|5174722|ref|NM_006114.1|[5174722]; 164: NM_006157, “Homo
sapiens NEL-like 1 (chicken) (NELL1), mRNA”, gi|5453763|ref|NM_006157.1|[5453763]; 165:
NM_006172, “Homo sapiens natriuretic peptide precursor A (NPPA), mRNA”,
gi|23510318|ref|NM_006172.1|[23510318]; 166: NM_006196, “Homo sapiens poly(rC) binding
protein 1 (PCBP1), mRNA”, gi|14141164|ref|NM_006196.2|[14141164]; 167: NM_006205,
“Homo sapiens phosphodiesterase 6H, cGMLP-specific, cone, gamma (PDE6H), mRNA”,
gi|5453867|ref|NM_006205.1|[5453867]; 168: NM_006228, “Homo sapiens prepronociceptin
(PNOC), mRNA”, gi|11079650|ref|NM_006228.2|[11079650]; 169: NM_006261, “Homo
sapiens prophet of Pit1, paired-like homeodomain transcription factor”, “(PROP1), mRNA”,
gi|40254838|ref|NM_006261.2|[40254838]; 170: NM_006289, “Homo sapiens talin 1 (TLN1),
mRNA”, gi|16753232|ref|NM_006289.2|[16753232]; 171: NM_006365, “Homo sapiens
transcriptional activator of the c-fos promoter (CROC4), mRNA”,
gi|5453624|ref|NM_006365.1|[5453624]; 172: NM_006368, “Homo sapiens cAMP responsive
element binding protein 3 (CREB3), mRNA”, gi|38327637|ref|NM_006368.4|[38327637]; 173:
NM_006399, “Homo sapiens basic leucine zipper transcription factor, ATF-like (BATF),
mRNA”, gi|18375640|ref|NM_006399.2|[18375640]; 174: NM_006477, “Homo sapiens RAS-
related on chromosome 22 (RRP22), mRNA”, gi|42476128|ref|NM_006477.2|[42476128]; 175:
NM_006698, “Homo sapiens bladder cancer associated protein (BLCAP), mRNA”,
gi|5729737|ref|NM_006698.1|[5729737]; 176: NM_006747, “Homo sapiens signal-induced
proliferation-associated gene 1 (SIPA1), transcript”, “variant 2, mRNA”,
gi|24497626|ref|NM_006747.2|[24497626]; 177: NM_006764, “Homo sapiens interferon-related
developmental regulator 2 (IFRD2), mRNA”, gi|21361365|ref|NM_006764.2|[21361365]; 178:
NM_006813, “Homo sapiens proline-rich nuclear receptor coactivator 1 (PNRC1), mRNA”,
gi|5802981|ref|NM_006813.1|[5802981]; 179: NM_006841, “Homo sapiens solute carrier
family 38, member 3 (SLC38A3), mRNA”, gi|40795668|ref|NM_006841.3|[40795668]; 180:
NM_006876, “Homo sapiens UDP-GlcNAc:betaGal beta-1,3-N-acetylglucosaminyltransferase
6”, “(B3GNT6), mRNA”, gi|5802983|ref|NM_006876.1|[5802983]; 181: NM_006917, “Homo
sapiens retinoid X receptor, gamma (RXRG), mRNA”,
gi|21361386|ref|NM_006917.2|[21361386]; 182: NM_006923, “Homo sapiens stromal cell-
derived factor 2 (SDF2), mRNA”, gi|14141194|ref|NM_006923.2|[14141194]; 183: NM_006946
“Homo sapiens spectrin, beta, non-erythrocytic 2 (SPTBN2), mRNA”,
gi|5902121|ref|NM_006946.1|[5902121]; 184: NM_006982, “Homo sapiens cartilage paired-
class homeoprotein 1 (CART1), mRNA”, gi|5901917|ref|NM_006982.1|[5901917]; 185:
NM_006998, “Homo sapiens secretagogin, EF-hand calcium binding protein (SCGN), mRNA”,
gi|15055536|ref|NM_006998.2|[15055536]; 186: NM_007022, “Homo sapiens putative tumor
suppressor 101F6 (101F6), mRNA”, gi|31541779|ref|NM_007022.3|[31541779]; 187:
NM_007046, “Homo sapiens elastin microfibril interfacer 1 (EMILIN1), mRNA”,
gi|5901943|ref|NM_007046.1|[5901943]; 188: NM_007076,, ref|NM_007076.2|[42794619];
189: NM_007149, “Homo sapiens zinc finger protein 184 (Kruppel-like) (ZNF184), mRNA”,
gi|24307934|ref|NM_007149.1|[24307934]; 190: NM_007357, “Homo sapiens component of
oligomeric golgi complex 2 (COG2), mRNA”, gi|6678675|ref|NM_007357.1|[6678675]; 191:
NM_012105, “Homo sapiens beta-site APP-cleaving enzyme 2 (BACE2), transcript variant a,
mRNA”, gi|21040358|ref|NM_012105.3|[21040358]; 192: NM_012164, “Homo sapiens F-box
and WD-40 domain protein 2 (FBXW2), mRNA”, gi|7549806|ref|NM_012164.2|[7549806]; 193:
NM_012168, “Homo sapiens F-box only protein 2 (FBXO2), mRNA”,
gi|15812197|ref|NM_012168.2|[15812197]; 194: NM_012191, “Homo sapiens putative tumor
suppressor (FUS2), mRNA”, gi|6912379|ref|NM_012191.1|[6912379]; 195: NM_012204,
“Homo sapiens general transcription factor IIIC, polypeptide 4, 90 kDa (GTF3C4),”, mRNA,
gi|6912399|ref|NM_012204.1|[6912399]; 196: NM_012285, “Homo sapiens potassium voltage-
gated channel, subfamily H (eag-related), member”, “4 (KCNH4), mRNA”,
gi|6912445|ref|NM_012285.1|[6912445]; 197: NM_012311, “Homo sapiens KIN, antigenic
determinant of recA protein homolog (mouse) (KIN),”, mRNA,
gi|40068516|ref|NM_012311.2|[40068516]; 198: NM_012430, “Homo sapiens SEC22 vesicle
trafficking protein-like 2 (S. cerevisiae) (SEC22L2),”, mRNA,
gi|14591918|ref|NM_012430.2|[14591918]; 199: NM_012459, Homo sapiens translocase of
inner mitochondrial membrane 8 homolog B (yeast), “(TIMM8B), mRNA”,
gi|6912711|ref|NM_012459.1|[6912711]; 200: NM_012460, Homo sapiens translocase of inner
mitochondrial membrane 9 homolog (yeast), “(TIMM9), mRNA”,
gi|21359892|ref|NM_012460.2|[21359892]; 201: NM_012482, “Homo sapiens zinc finger
protein 281 (ZNF281), mRNA”, gi|40255235|ref|NM_012482.3|[40255235]; 202: NM_013235,
“Homo sapiens nuclear RNase III Drosha (RNASE3L), mRNA”,
gi|21359821|ref|NM_013235.2|[21359821]; 203: NM_013333, “Homo sapiens epsin 1 (EPN1),
mRNA”, gi|41350200|ref|NM_013333.2|[41350200]; 204: NM_013335, “Homo sapiens GDP-
mannose pyrophosphorylase A (GMPPA), mRNA”, gi|31881778|ref|NM_013335.2|[31881778];
205: NM_013343, “Homo sapiens loss of heterozygosity, 3, chromosomal region 2, gene A
(LOH3CR2A),”, mRNA, gi|7106370|ref|NM_013343.1|[7106370]; 206: NM_013387, “Homo
sapiens ubiquinol-cytochrome c reductase complex (7.2 kD) (HSPC051), mRNA”,
gi|41281884|ref|NM_013387.2|[41281884]; 207: NM_013403, “Homo sapiens striatin,
calmodulin binding protein 4 (STRN4), mRNA”, gi|7019572|ref|NM_013403.1|[7019572]; 208:
NM_013441, “Homo sapiens Down syndrome critical region gene 1-like 2 (DSCR1L2),
mRNA”, gi|38455419|ref|NM_013441.2|[38455419]; 209: NM_014099,,
ref|NM_014099.1|[7662610], This record was temporarily removed by RefSeq staff for
additional review.,, 210: NM_014124,, ref|NM_014124.1|[7662541], This record was
temporarily removed by RefSeq staff for additional review.,, 211: NM_014165, “Homo sapiens
chromosome 6 open reading frame 66 (C6orf66), mRNA”,
gi|7661785|ref|NM_014165.1|[7661785]; 212: NM_014236, “Homo sapiens glyceronephosphate
O-acyltransferase (GNPAT), mRNA”, gi|7657133|ref|NM_014236.1|[7657133]; 213:
NM_014301, “Homo sapiens nitrogen fixation cluster-like (NIFU), mRNA”,
gi|24307952|ref|NM_014301.1|[24307952]; 214: NM_014342, “Homo sapiens mitochondrial
carrier homolog 2 (C. elegans) (MTCH2), nuclear gene”, “encoding mitochondrial protein,
mRNA”, gi|40254847|ref|NM_014342.2|[40254847]; 215: NM_014348, “Homo sapiens
POM121 membrane glycoprotein-like 1 (rat) (POM121L1), mRNA”,
gi|7657468|ref|NM_014348.1|[7657468]; 216: NM_014453, “Homo sapiens putative breast
adenocarcinoma marker (32 kD) (BC-2), transcript”, “variant 1, mRNA”,
gi|38372936|ref|NM_014453.2|[38372936]; 217: NM_014548, “Homo sapiens tropomodulin 2
(neuronal) (TMOD2), mRNA”, gi|40789262|ref|NM_014548.2|[40789262]; 218: NM_014606,,
ref|NM_014606.1|[7657151], This record was temporarily removed by RefSeq staff for
additional review.,, 219: NM_014662,, ref|NM_014662.1|[7662221], This record was
temporarily removed by RefSeq staff for additional review.,, 220: NM_014674,,
ref|NM_014674.1|[7662001], This record was temporarily removed by RefSeq staff for
additional review.,, 221: NM_014748, “Homo sapiens sorting nexin 17 (SNX17), mRNA”,
gi|23238249|ref|NM_014748.2|[23238249]; 222: NM_014786, “Homo sapiens Rho guanine
nucleotide exchange factor (GEF) 17 (ARHGEF17), mRNA”,
gi|21361457|ref|NM_014786.2|[21361457]; 223: NM_014813,, ref|NM_014813.1|[7662319],
This record was temporarily removed by RefSeq staff for additional review.,, 224: NM_014814,
“Homo sapiens proteasome regulatory particle subunit p44S10 (p44S10), mRNA”,
gi|7661913|ref|NM_014814.1|[7661913]; 225: NM_014901, “Homo sapiens ring finger protein
44 (RNF44), mRNA”, gi|42718018|ref|NM_014901.4|[42718018]; 226: NM_014907, “Homo
sapiens FERM and PDZ domain containing 1 (FRMPD1), mRNA”,
gi|7662415|ref|NM_014907.1|[7662415]; 227: NM_015089, “Homo sapiens p53-associated
parkin-like cytoplasmic protein (PARC), mRNA”, gi|24307990|ref|NM_015089.1|[24307990];
228: NM_015163, “Homo sapiens tripartite motif-containing 9 (TRIM9), transcript variant 1,
mRNA”, gi|29543553|ref|NM_015163.3|[29543553]; 229: NM_015343, “Homo sapiens dullard
homolog (Xenopus laevis) (DULLARD), mRNA”, gi|34222318|ref|NM_015343.3|[34222318];
230: NM_015362,, ref|NM_015362.3|[44662829]; 231: NM_015372, “Homo sapiens
hypothetical protein HSN44A4A (HSN44A4A), mRNA”,
gi|7661723|ref|NM_015372.1|[7661723]; 232: NM_015480, “Homo sapiens poliovirus receptor-
related 3 (PVRL3), mRNA”, gi|[11386198|ref|NM_015480.1|[1386198]; 233: NM_015623,,
ref|NM_015623.2|[32306520], This record was temporarily removed by RefSeq staff for
additional review.,, 234: NM_015710, “Homo sapiens glioma tumor suppressor candidate
region gene 2 (GLTSCR2), mRNA”, gi|21359905|ref|NM_015710.2|[21359905]; 235:
NM_015926, “Homo sapiens putative secreted protein ZSIG11 (ZSIG11), mRNA”,
gi|34147580|ref|NM_015926.3|[34147580]; 236: NM_015964, “Homo sapiens brain specific
protein (CGI-38), mRNA”, gi|7706275|ref|NM_015964.1|[7706275]; 237: NM_016004, “Homo
sapiens chromosome 20 open reading frame 9 (C20orf9), mRNA”,
gi|7705768|ref|NM_016004.1|[7705768]; 238: NM_016067, “Homo sapiens mitochondrial
ribosomal protein S18C (MRPS18C), nuclear gene”, “encoding mitochondrial protein, mRNA”,
gi|7705629|ref|NM_016067.1|[7705629]; 239: NM_016082, “Homo sapiens CDK5 regulatory
subunit associated protein 1 (CDK5RAP1), transcript”, “variant 2, mRNA”,
gi|28872783|ref|NM_016082.3|[28872783]; 240: NM_016090, “Homo sapiens RNA binding
motif protein 7 (RBM7), mRNA”, gi|31543547|ref|NM_016090.2|[31543547]; 241: NM_016187,
“Homo sapiens bridging integrator 2 (BIN2), mRNA”,
gi|7705295|ref|NM_016187.1|[7705295]; 242: NM_016210, “Homo sapiens g20 protein
(LOC51161), mRNA”, gi|31543080|ref|NM_016210.2|[31543080]; 243: NM_016231, “Homo
sapiens nemo like kinase (NLK), mRNA”, gi|42734431|ref|NM_016231.2|[42734431]; 244:
NM_016324, “Homo sapiens zinc finger protein 274 (ZNF274), transcript variant ZNF274b,
mRNA”, gi|19743797|ref|NM_016324.2|[19743797]; 245: NM_016368, “Homo sapiens myoinositol
1-phosphate synthase A1 (ISYNA1), mRNA”,
gi|21902536|ref|NM_016368.3|[21902536]; 246: NM_017582, “Homo sapiens ubiquitin-
conjugating enzyme E2Q (putative) (UBE2Q), mRNA”,
gi|38045949|ref|NM_017582.5|[38045949]; 247: NM_017704, “Homo sapiens fetal globin-
inducing factor (FGIF), mRNA”, gi|41350197|ref|NM_017704.2|[41350197]; 248: NM_017740,
“Homo sapiens zinc finger, DHHC domain containing 7 (ZDHHC7), mRNA”,
gi|8923254|ref|NM_017740.1|[8923254]; 249: NM_017745, “Homo sapiens BCL6 co-repressor
(BCOR), transcript variant 1, mRNA”, gi|21071036|ref|NM_017745.4|[21071036]; 250:
NM_017746, “Homo sapiens testis expressed gene 10 (TEX10), mRNA”,
gi|8923268|ref|NM_017746.1|[8923268]; 251: NM_017806, “Homo sapiens hypothetical
protein FLJ20406 (FLJ20406), mRNA”, gi|8923377|ref|NM_017806.1|[8923377]; 252:
NM_017847, “Homo sapiens chromosome 1 open reading frame 27 (C1orf27), mRNA”,
gi|20127566|ref|NM_017847.2|[20127566]; 253: NM_017901, “Homo sapiens two pore
segment channel 1 (TPCN1), mRNA”, gi|29725621|ref|NM_017901.3|[29725621]; 254:
NM_017915, “Homo sapiens hypothetical protein FLJ20641 (FLJ20641), mRNA”,
gi|8923595|ref|NM_017915.1|[8923595]; 255: NM_017941, “Homo sapiens lung cancer-related
protein 8 (HLC-8), mRNA”, gi|34222156|ref|NM_017941.3|[34222156]; 256: NM_017991,
“Homo sapiens hypothetical protein FLJ10081 (FLJ10081), mRNA”,
gi|21361733|ref|NM_017991.3|[21361733]; 257: NM_018026, “Homo sapiens phosphofurin
acidic cluster sorting protein 1 (PACS1), mRNA”, gi|30089915|ref|NM_018026.2|[30089915];
258: NM_018058, “Homo sapiens cartilage acidic protein 1 (CRTAC1), mRNA”,
gi|42415498|ref|NM_018058.2|[42415498]; 259: NM_018163, “Homo sapiens hypothetical
protein FLJ10634 (FLJ10634), mRNA”, gi|8922562|ref|NM_018163.1|[8922562]; 260:
NM_018195, “Homo sapiens hypothetical protein FLJ10726 (FLJ10726), mRNA”,
gi|40254918|ref|NM_018195.2|[40254918]; 261: NM_018206, “Homo sapiens vacuolar protein
sorting 35 (yeast) (VPS35), mRNA”, gi|41352714|ref|NM_018206.3|[41352714]; 262:
NM_018233, “Homo sapiens hypothetical protein FLJ10826 (FLJ10826), mRNA”,
gi|42476029|ref|NM_018233.2|[42476029]; 263: NM_018245, “Homo sapiens hypothetical
protein FLJ10851 (FLJ10851), mRNA”, gi|8922715|ref|NM_018245.1|[8922715]; 264:
NM_018261, “Homo sapiens SEC3-like 1 (S. cerevisiae) (SEC3L1), transcript variant 1,
mRNA”, gi|30410719|ref|NM_018261.2|[30410719]; 265: NM_018303, “Homo sapiens SEC5-
like 1 (S. cerevisiae) (SEC5L1), mRNA”, gi|30581133|ref|NM_018303.4|[30581133]; 266:
NM_018327, “Homo sapiens chromosome 20 open reading frame 38 (C20orf38), mRNA”,
gi|8922874|ref|NM_018327.1|[8922874]; 267: NM_018431, “Homo sapiens docking protein 5
(DOK5), transcript variant 1, mRNA”, gi|29544725|ref|NM_018431.2|[29544725]; 268:
NM_018459,, ref|NM_018459.1|[8922103], This record was replaced or removed. See revision
history for details.,, 269: NM_018465, “Homo sapiens chromosome 9 open reading frame 46
(C9orf46), mRNA”, gi|8923931|ref|NM_018465.1|[8923931]; 270: NM_018484, “Homo
sapiens solute carrier family 22 (organic anion/cation transporter), member”, “11 (SLC22A11),
mRNA”, gi|24497483|ref|NM_018484.2|[24497483]; 271: NM_018584, “Homo sapiens
calcium/calmodulin-dependent protein kinase II (CaMKIINalpha), mRNA”,
gi|31324542|ref|NM_018584.4|[31324542]; 272: NM_018641, “Homo sapiens carbohydrate
(chondroitin 4) sulfotransferase 12 (CHST12), mRNA”,
gi|20070291|ref|NM_018641.2|[20070291]; 273: NM_018947, “Homo sapiens cytochrome c,
somatic (CYCS), nuclear gene encoding mitochondrial”, “protein, mRNA”,
gi|34328939|ref|NM_018947.4|[34328939]; 274: NM_018957, “Homo sapiens SH3-domain
binding protein 1 (SH3BP1), mRNA”, gi|15147251|ref|NM_018957.2|[15147251]; 275:
NM_018959, “Homo sapiens DAZ associated protein 1 (DAZAP1), transcript variant 2,
mRNA”, gi|25470885|ref|NM_018959.2|[25470885]; 276: NM_018993, “Homo sapiens Ras and
Rab interactor 2 (RIN2), mRNA”, gi|35493905|ref|NM_018993.2|[35493905]; 277: NM_019063,
“Homo sapiens echinoderm microtubule associated protein like 4 (EML4), mRNA”,
gi|19923496|ref|NM_019063.2|[19923496]; 278: NM_020170, “Homo sapiens hypothetical
protein from EUROIMAGE 2021883 (LOC56926), mRNA”,
gi|24308184|ref|NM_020170.1|[24308184]; 279: NM_020188, “Homo sapiens DC13 protein
(DC13), mRNA”, gi|42476040|ref|NM_020188.2|[42476040]; 280: NM_020228, “Homo
sapiens PR domain containing 10 (PRDM10), transcript variant 1, mRNA”,
gi|41349457|ref|NM_020228.2|[41349457]; 281: NM_020418, “Homo sapiens poly(rC) binding
protein 4 (PCBP4), transcript variant 1, mRNA”, gi|14670367|ref|NM_020418.2|[14670367];
282: NM_020465, “Homo sapiens NDRG family member 4 (NDRG4), mRNA”,
gi|14165263|ref|NM_020465.1|[14165263]; 283: NM_020999, “Homo sapiens neurogenin 3
(NEUROG3), mRNA”, gi|10337610|ref|NM_020999.1|[10337610]; 284: NM_021018, “Homo
sapiens histone 1, H3f (HIST1H3F), mRNA”, gi|21396497|ref|NM_021018.2|[21396497]; 285:
NM_021025, “Homo sapiens T-cell leukemia, homeobox 3 (TLX3), mRNA”,
gi|10440563|ref|NM_021025.1|[10440563]; 286: NM_021062, “Homo sapiens histone 1, H2bb
(HIST1H2BB), mRNA”, gi|19924303|ref|NM_021062.2|[19924303]; 287: NM_021161, “Homo
sapiens potassium channel, subfamily K, member 10 (KCNK10), transcript”, “variant 1,
mRNA”, gi|20143942|ref|NM_021161.3|[20143942]; 288: NM_021174, “Homo sapiens p30
DBC protein (DBC-1), transcript variant 1, mRNA”, gi|40548406|ref|NM_021174.4|[40548406];
289: NM_021184, “Homo sapiens chromosome 6 open reading frame 47 (C6orf47), mRNA”,
gi|10863984|ref|NM_021184.1|[10863984]; 290: NM_021249, “Homo sapiens sorting nexin 6
(SNX6), transcript variant 1, mRNA”, gi|23111048|ref|NM_021249.2|[23111048]; 291:
NM_021259, “Homo sapiens transmembrane protein 8 (five membrane-spanning domains)
(TMEM8),”, mRNA, gi|10864068|ref|NM_021259.1|[10864068]; 292: NM_021812, “Homo
sapiens blepharophimosis, epicanthus inversus and ptosis, candidate 1”, “(BPESC1), mRNA”,
gi|11141882|ref|NM_021812.1|[11141882]; 293: NM_021830, “Homo sapiens progressive
external ophthalmoplegia 1 (PEO1), mRNA”, gi|39725941|ref|NM_021830.3|[39725941]; 294:
NM_021833, “Homo sapiens uncoupling protein 1 (mitochondrial, proton carrier) (UCP1),”,
“nuclear gene encoding mitochondrial protein, mRNA”,
gi|21614550|ref|NM_021833.3|[21614550]; 295: NM_021926, “Homo sapiens aristaless-like
homeobox 4 (ALX4), mRNA”, gi|11496266|ref|NM_021926.1|[11496266]; 296: NM_021934,
“Homo sapiens hypothetical protein FLJ11773 (FLJ11773), mRNA”,
gi|34222337|ref|NM_021934.3|[34222337]; 297: NM_022039, “Homo sapiens split hand/foot
malformation (ectrodactyly) type 3 (SHFM3), mRNA”,
gi|24475655|ref|NM_022039.2|[24475655]; 298: NM_022054, “Homo sapiens potassium
channel, subfamily K, member 13 (KCNK13), mRNA”,
gi|16306554|ref|NM_022054.2|[16306554]; 299: NM_022064, “Homo sapiens ring finger
protein 123 (RNF123), mRNA”, gi|37588868|ref|NM_022064.2|[37588868]; 300: NM_022082,
“Homo sapiens chromosome 20 open reading frame 59 (C20orf59), mRNA”,
gi|31542262|ref|NM_022082.2|[31542262]; 301: NM_022114, “Homo sapiens PR domain
containing 16 (PRDM16), transcript variant 1, mRNA”,
gi|41349469|ref|NM_022114.2|[41349469]; 302: NM_022120, “Homo sapiens 3-oxoacid CoA
transferase 2 (OXCT2), mRNA”, gi|11545840|ref|NM_022120.1|[11545840]; 303: NM_022135,
“Homo sapiens popeye domain containing 2 (POPDC2), mRNA”,
gi|22209003|ref|NM_022135.2|[22209003]; 304: NM_022354, “Homo sapiens spermatogenesis
associated 1 (SPATA1), mRNA”, gi|11641266|ref|NM_022354.1|[11641266]; 305: NM_022452,
“Homo sapiens fibrosin 1 (FBS1), mRNA”, gi|11967986|ref|NM_022452.1|[11967986]; 306:
NM_022494, “Homo sapiens zinc finger, DHHC domain containing 6 (ZDHHC6), mRNA”,
gi|11968052|ref|NM_022494.1|[11968052]; 307: NM_022727, “Homo sapiens HpaII tiny
fragments locus 9C (HTF9C), transcript variant 2, mRNA”,
gi|21361611|ref|NM_022727.3|[21361611]; 308: NM_022754, “Homo sapiens sideroflexin 1
(SFXN1), mRNA”, gi|40255158|ref|NM_022754.4|[40255158]; 309: NM_022765, Homo
sapiens NEDD9 interacting protein with calponin homology and LIM domains, “(NICAL),
mRNA”, gi|20127615|ref|NM_022765.2|[20127615]; 310: NM_022766, “Homo sapiens
ceramide kinase (CERK), transcript variant 1, mRNA”,
gi|32967301|ref|NM_022766.4|[32967301]; 311: NM_023933, “Homo sapiens hypothetical
protein MGC2494 (MGC2494), mRNA”, gi|13027599|ref|NM_023933.1|[13027599]; 312:
NM_024034, Homo sapiens ganglioside-induced differentiation-associated protein 1-like 1,
“(GDAP1L1), mRNA”, gi|30581159|ref|NM_024034.3|[30581159]; 313: NM_024057, “Homo
sapiens nucleoporin Nup37 (Nup37), mRNA”, gi|34222120|ref|NM_024057.2|[34222120]; 314:
NM_024294, “Homo sapiens hypothetical protein MGC4614 (MGC4614), mRNA”,
gi|13236513|ref|NM_024294.1|[13236513]; 315: NM_024323, “Homo sapiens hypothetical
protein MGC11271 (MGC11271), mRNA”, gi|31543147|ref|NM_024323.3|[31543147]; 316:
NM_024506, “Homo sapiens galactosidase, beta 1-like (GLB1L), mRNA”,
gi|40255042|ref|NM_024506.3|[40255042]; 317: NM_024523, “Homo sapiens GRIP and coiled-
coil domain-containing 1 (GCC1), mRNA”, gi|34305454|ref|NM_024523.5|[34305454]; 318:
NM_024546, “Homo sapiens chromosome 13 open reading frame 7 (C13orf7), mRNA”,
gi|21362045|ref|NM_024546.2|[21362045]; 319: NM_024589, “Homo sapiens leucine zipper
domain protein (FLJ22386), mRNA”, gi|13375778|ref|NM_024589.1|[13375778]; 320:
NM_024604, “Homo sapiens hypothetical protein FLJ21908 (FLJ21908), mRNA”,
gi|13375808|ref|NM_024604.1|[13375808]; 321: NM_024624, Homo sapiens SMC6 structural
maintenance of chromosomes 6-like 1 (yeast), “(SMC6L1), mRNA”,
gi|31543646|ref|NM_024624.2|[31543646]; 322: NM_024630, “Homo sapiens zinc finger,
DHHC domain containing 14 (ZDHHC14), mRNA”, gi|24371240|ref|NM_024630.2|[24371240];
323: NM_024643, “Homo sapiens chromosome 14 open reading frame 140 (C14orf140),
mRNA”, gi|13375882|ref|NM_024643.1|[13375882]; 324: NM_024696, “Homo sapiens
hypothetical protein FLJ23058 (FLJ23058), mRNA”,
gi|13375978|ref|NM_024696.1|[13375978]; 325: NM_024728, “Homo sapiens chromosome 7
open reading frame 10 (C7orf10), mRNA”, gi|13376041|ref|NM_024728.1|[13376041]; 326:
NM_024731, “Homo sapiens chromosome 16 open reading frame 44 (C16orf44), mRNA”,
gi|31542245|ref|NM_024731.2|[31542245]; 327: NM_024778, “Homo sapiens ring finger
protein 127 (RNF127), mRNA”, gi|37622895|ref|NM_024778.3|[37622895]; 328: NM_024783,
“Homo sapiens hypothetical protein FLJ23598 (FLJ23598), mRNA”,
gi|31657118|ref|NM_024783.2|[31657118]; 329: NM_024799, “Homo sapiens hypothetical
protein FLJ13224 (FLJ13224), mRNA”, gi|13376172|ref|NM_024799.1|[13376172]; 330:
NM_024827, “Homo sapiens histone deacetylase 11 (HDAC11), mRNA”,
gi|13376227|ref|NM_024827.1|[13376227]; 331: NM_024958, “Homo sapiens chromosome 20
open reading frame 98 (C20orf98), mRNA”, gi|13376446|ref|NM_024958.1|[13376446]; 332:
NM_025079, “Homo sapiens hypothetical protein FLJ23231 (FLJ23231), mRNA”,
gi|13376631|ref|NM_025079.1|[13376631]; 333: NM_025137, “Homo sapiens hypothetical
protein FLJ21439 (FLJ21439), mRNA”, gi|33636747|ref|NM_025137.2|[33636747]; 334:
NM_025140, “Homo sapiens limkain beta 2 (FLJ22471), mRNA”,
gi|13376724|ref|NM_025140.1|[13376724]; 335: NM_025212, “Homo sapiens CXXC finger 4
(CXXC4), mRNA”, gi|13376815|ref|NM_025212.1|[13376815]; 336: NM_025236, “Homo
sapiens ring finger protein 39 (RNF39), transcript variant 1, mRNA”,
gi|25777714|ref|NM_025236.2|[25777714]; 337: NM_030804,, ref|NM_030804.1|[13540591],
This record was temporarily removed by RefSeq staff for additional review.,, 338: NM_030818,
“Homo sapiens hypothetical protein MGC10471 (MGC10471), mRNA”,
gi|34147391|ref|NM_030818.2|[34147391]; 339: NM_031219, “Homo sapiens hypothetical
protein MGC12904 (MGC12904), mRNA”, gi|31377665|ref|NM_031219.2|[31377665]; 340:
NM_031284, “Homo sapiens ATP-dependent glucokinase (ADP-GK), mRNA”,
gi|31542508|ref|NM_031284.3|[31542508]; 341: NM_031298, “Homo sapiens hypothetical
protein MGC2963 (MGC2963), mRNA”, gi|13775219|ref|NM_031298.1|[13775219]; 342:
NM_031450, “Homo sapiens hypothetical protein p5326 (P5326), mRNA”,
gi|31543378|ref|NM_031450.2|[31543378]; 343: NM_032179, “Homo sapiens hypothetical
protein FLJ20542 (FLJ20542), mRNA”, gi|14149862|ref|NM_032179.1|[14149862]; 344:
NM_032204, “Homo sapiens ASC-1 complex subunit P100 (ASC1p100), mRNA”,
gi|34147616|ref|NM_032204.3|[34147616]; 345: NM_032209, “Homo sapiens hypothetical
protein FLJ21777 (FLJ21777), mRNA”, gi|14149905|ref|NM_032209.1|[14149905]; 346:
NM_032338, “Homo sapiens hypothetical protein MGC14817 (MGC14817), mRNA”,
gi|31543151|ref|NM_032338.2|[31543151]; 347: NM_032348, “Homo sapiens hypothetical
protein MGC3047 (MGC3047), mRNA”, gi|39725651|ref|NM_032348.2|[39725651]; 348:
NM_032389, “Homo sapiens zinc finger protein 289, ID1 regulated (ZNF289), mRNA”,
gi|31543982|ref|NM_032389.2|[31543982]; 349: NM_032842, “Homo sapiens hypothetical
protein FLJ14803 (FLJ14803), mRNA”, gi|14249557|ref|NM_032842.1|[14249557]; 350:
NM_130463, “Homo sapiens ATPase, H+ transporting, lysosomal 13 kDa, V1 subunit G isoform
2”, “(ATP6V1G2), transcript variant 1, mRNA”, gi|20357536|ref|NM_130463.2|[20357536];
351: NM_144718, “Homo sapiens hypothetical protein AY099107 (LOC152185), mRNA”,
gi|40255074|ref|NM_144718.2|[40255074]; 352: NM_145804, “Homo sapiens ankyrin repeat
and BTB (POZ) domain containing 2 (ABTB2), mRNA”,
gi|21956638|ref|NM_145804.1|[21956638]; 353: NM_153045, “Homo sapiens DKFZp547P234
protein (DKFZp547P234), mRNA”, gi|33356141|ref|NM_153045.2|[33356141]; 354:
NM_153354, “Homo sapiens hypothetical protein MGC33214 (MGC33214), mRNA”,
gi|34222213|ref|NM_153354.2|[34222213],,

1. A method of modulating a biological response in a cell, the method comprising contacting the cell with at least one agent that modulates the expression or activity of Errα or Gabp, wherein the biological response is
(a) expression of at least one OXPHOS gene;
(b) mitochondrial biogenesis;
(c) expression of Nuclear Respiratory Factor 1 (NRF-1);
(d) β-oxidation of fatty acids;
(e) total mitochondrial respiration;
(f) uncoupled respiration;
(g) mitochondrial DNA replication;
(h) expression of mitochondrial enzymes; or
(i) skeletal muscle fiber-type switching.

2. The method of claim 1, wherein the agent increases at least one of the biological responses.

3. The method of claim 1, wherein the agent modulates the formation of a complex between a PGC-1 polypeptide and (i) an Errα polypeptide; or (ii) a Gabp polypeptide.

4. The method of claim 3, wherein the agent increases the formation of the complex.

5. The method of claim 1, wherein the agent is an Errα antagonist or an Errα agonist.

6. The method of claim 1, wherein the agent modulates the expression level or the transcriptional activity of an Errα polypeptide, a Gabp polypeptide, or of both.

7. The method of claim 1, comprising contacting the cell with two agents, wherein one agent modulates the expression or activity of Errα and the other agent modulates the expression or activity of Gabp.

8-10. (canceled)

11. The method of claim 1, wherein the cell is in an organism.

12. The method of claim 11, wherein the organism is a mammal.

13. The method of claim 12, wherein the mammal is a human.

14. The method of claim 13, wherein the human is afflicted with a disorder characterized by reduced mitochondrial activity.

15. The method of claim 14, wherein the disorder is diabetes, obesity, cardiac myopathy, aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRF), NARP (Neuropathy; Ataxia; Retinitis Pigmentosa), MNGIE (Myopathy and external ophthalmoplegia, neuropathy; gastro-intestinal encephalopathy, Kearns-Sayre disease, Pearson's Syndrome, PEO (Progressive External Ophthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, mitochondrial diabetes and deafness (MIDD), or mitochondrial DNA depletion syndrome.

16. The method of claim 1, wherein the cell is a skeletal muscle cell.

17. A method of determining if an agent is a potential agent for the treatment of a disorder that is characterized by glucose intolerance, insulin resistance or reduced mitochondrial function, the method comprising determining if the agent increases:
(i) the expression or activity of Errα or Gabp in a cell; or
(ii) the formation of a complex between a PGC-1 polypeptide and (i) an Errα polypeptide; or (ii) a Gabp polypeptide;
wherein an agent that increases (i) or (ii) is a potential target for the treatment of the disorder.

18. (canceled)

19. The method of claim 17, wherein the agent increases the formation of the complex, and wherein the agent increases the biological response.

20. The method of claim 19, wherein the agent decreases the formation of the complex, and wherein the agent decreases the biological response.

21. The method of claim 18, wherein the contacting step is performed on a cell.

22-34. (canceled)

35. A method of reducing the metabolic rate of a subject in need thereof, the method comprising administering to the subject a therapeutically effective amount of an agent which decreases the expression or activity of at least one of the following:
(i) Errα;
(ii) Gabpa;
(iii) a gene having an Errα binding site, a Gabpa binding site, or both; or
(iv) a transcriptional activator which binds to an Errα binding site or to a Gabpa binding site;
thereby reducing the metabolic rate of the patient.

36. The method of claim 35, wherein the subject is afflicted with a viral infection or with cancer.

37. The method of claim 35, wherein the viral infection is a human immunodeficiency virus infection.

38. The method of claim 35, wherein the subject is afflicted with cancer cachexia, pulmonary cachexia, cardiac cachexia, Russell's Diencephalic Cachexia, or chronic renal insufficiency.

39-41. (canceled)

42. A method of identifying a susceptibility locus for a disorder that is characterized by reduced mitochondrial function, glucose intolerance, or insulin intolerance in a subject, the method comprising
(i) identifying at least one polymorphisms in a gene, or linked to a gene, wherein the gene (a) has an Errα binding site, a Gabpa binding site, or both; or (b) is Errα, Gabpa, or Gabpb;
(ii) determining if at least one polymorphism is associated with the incidence of the disorder,
wherein if a polymorphism is associated with the incidence of the disorder then the gene having the polymorphism, or the gene to which the polymorphism is linked, is a susceptibility locus.

43. The method of claim 42, wherein the gene is anyone of the gene listed on Tables 10-12.

44. The method of claim 42, wherein the disorder is a metabolic disorder.

45. The method of claim 44, wherein the disorder is diabetes or obesity.

46. The method of claim 44, wherein the metabolic disorder is a disorder associated with aberrant lipogenesis.

47. A method of determining if a subject is at risk of developing a disorder which is characterized by reduced mitochondrial function, the method comprising determining if a gene from the subject contains a mutation which reduces the function of the gene, wherein the gene has an Errα binding site, a Gapba binding site, or both, wherein if a gene from the subject contains the mutation then the subject is at risk of developing the disorder.

48. The method of claim 47, wherein the mutation reduces the function of the gene.

49-77. (canceled)

78. A method of detecting statistically-significant differences in the expression level of at least one biomarker belonging to a biomarker set, between the members of a first and of a second experimental group, comprising:
(a) obtaining a biomarker sample from members of the first and the second experimental groups;
(b) determining, for each biomarker sample, the expression levels of at least one biomarker belonging to the biomarker set and of at least one biomarker not belonging to the set;
(c) generating a rank order of each biomarker according to a difference metric of its expression level in the first experimental group compared to the second experimental group;
(d) calculating an experimental enrichment score for the biomarker set by applying a non parametric statistic; and
(e) comparing the experimental enrichment score with a distribution of randomized enrichment scores to calculate the fraction of randomized enrichment scores greater than the experimental enrichment score, wherein a low fraction indicates a statistically-significant difference in the expression level of the biomarker set between the members of the first and of the second experimental group.

79-92. (canceled)

93. A method of identifying an agent that regulates expression of OXPHOS-CR genes, the method comprising
(a) contacting (i) an agent to be assessed for its ability to regulate expression of OXPHOS-CR genes with (ii) a test cell; and
(b) determining whether the expression of at least two OXPHOS-CR gene products show a coordinate change in the test cell compared to an appropriate control,
wherein a coordinate change in the expression of the OXPHOS-CR gene products indicates that the agent regulates the expression of OXPHOS-CR genes.

94-105. (canceled)
